(19) United States
(12) Patent Application Publication
(10) Pub. No.: US 2025/0302930 A1
(43) Pub. Date: Oct. 2, 2025

(54) IMMUNOTHERAPY TARGETING TUMOR TRANSPOSABLE ELEMENT DERIVED NEOANTIGENIC PEPTIDES IN GLIOBLASTOMA

(71) Applicants: INSTITUT CURIE, Paris (FR); INSERM (INSTITUT NATIONAL DE LA SANTE ET DE LA REC, Paris (FR); UNIVERSITE PARIS CITE, Paris (FR)

(72) Inventors: Sebastian AMIGORENA, Paris (FR); Christel GOUDOT, Le Blanc Mesnil (FR); Pierre Emmanuel BONTE, Lieusaint (FR); Antonela MERLOTTI IPPOLITO, Paris (FR); Yago ARRIBAS DE SANDOVAL, Paris (FR)

(21) Appl. No.: 18/849,761

(22) PCT Filed: Mar. 24, 2023
(86) PCT No.: PCT/EP2023/057700

§ 371 Date: Sep. 23, 2024

(30) Foreign Application Priority Data
Mar. 24, 2022 (EP) 22305355.4

(51) Int. Cl. A61K 39/00; A61K 39/395; A61P 35/00; C07K 14/47; C07K 14/725; C07K 16/28; C07K 16/30

(52) U.S. Cl. CPC A61K 39/0011; A61K 39/39558; A61P 35/00; C07K 14/4748; C07K 14/7051; C07K 16/2809; C07K 16/283; C07K 16/2878; C07K 16/30; C07K 2317/31

(57) ABSTRACT

The present disclosure provides shared neoantigenic peptides derived from the expression of tumor-specific transposable element, as well as nucleic acids, vaccines, antibodies and immune cells that can be used in cancer therapy.

### FIELD OF THE DISCLOSURE

The present disclosure provides shared neoantigenic peptides derived from the expression of tumor-specific transposable element, as well as nucleic acids, vaccines, antibodies and immune cells that can be used in cancer therapy.

### BACKGROUND

Harnessing the immune system to generate effective responses against tumors is a central goal of cancer immunotherapy.

Part of the effective immune response involves T lymphocytes specific for tumor antigens. T cell activation requires their interaction with antigen-presenting cells (APCs), commonly dendritic cells (DCs), expressing TCR-cognate peptides presented in the context of a major histocompatibility molecule (MHC) and co-stimulation signals. Neoplasms often contain infiltrating T lymphocytes reactive with tumor cells. Subsequently, activated T cells can recognize peptide-MHC complexes presented by all cell types, even malignant cells.

It is commonly accepted that T cells can control, and sometimes reject, solid tumors, especially after immune checkpoint blockade (ICB). Indeed, the development of checkpoint blockade therapy has provided means to bypass some of these mechanisms, leading to more efficient killing of cancer cells. The promising results yielded by this approach have opened up new avenues for the development of T cell-based immunotherapy.

The nature of the tumor antigens targeted by these T cells, however, remains partially unclear. After the identification of differentiation and tumor-testis antigens a few decades ago (Boon et al., J Exp Med, 1996, 183, 725-729, doi:10.1084/jem.183.3.725; Almeida et al., Nucleic Acids Res, 2009, 37, D816-819, doi:10.1093/nar/gkn673; Simpson et al., Nat Rev Cancer, 2005, 5, 615-625, doi:10.1038/nrc1669), a new family of antigens derived from passenger tumor mutations was discovered. Defined sets of mutations in single cells, before or after oncogenic transformation, are amplified by clonal expansion of tumor cells. This set of mutations that are now expressed in multiple tumor cells becomes “visible” to the immune system, and trigger T cell immune responses. Unlike differentiation and tumor testis antigens, mutational neo-antigens are by definition tumor-specific, and therefore recognized by the immune system as “non-self”. Clear evidence is available, including the high rate of clinical responses to ICB in patients with microsatellite instability (who bear very high numbers of point mutations in their tumors) or the correlation existing between the median number of mutations in cancer types and the rate of response ICB.

Several lines of evidence, however, also suggest that point mutations are not the only antigens seen by T cells on tumors. First, there are exceptions to the correlation between the frequency of mutations and the rates of response to ICB. RCC, for example has a mutational burden around 2 mutations per MB, and a response rate to ICB around 25%, as compared to squamous non-small cell lung cancer (LUSC), around 9 mutations/MB and a response rate to ICB of 17% (Yarchoan et al., N Engl J Med, 2017, 377, 2500-2501, doi:10.1056/NEJMc1713444; Yarchoan et al., JCI Insight, 2019, 4, doi:10.1172/jci.insight.126908). Second, at the level of individual patients, the number of mutations is not predictive of clinical responses to ICB. Third, tumor types with extremely low mutation burdens (and limited genomic instability), such as rhabdomyosarcoma show relatively high rates of clinical responses to ICB (McGrail et al., Ann Oncol, 2021, 32, 661-672, doi:10.1016/j.annonc.2021.02.006; Gromeier et al., Nat Commun, 2021, 12, 352, doi:10.1038/s41467-020-20469-6). Finally, there are multiple examples in the literature of T cell responses in patients to non-mutational antigens, including differentiation and tumor-testis antigens.

Non-coding genome-peptide antigens can also represent tumor-specific antigens. Different teams recently used proteogenomics, i.e.: experimental approaches based on a combination of transcriptomic and immunopeptidomics analyses, to search randomly for tumor-specific ORFs that encode peptides presented by MHC-I molecules on tumor cells (Laumont et al., Nat Commun, 2016, 7, 10238, doi:10.1038/ncomms10238; Chong et al., Nat Commun, 2020, 11, 1293, doi:10.1038/s41467-020-14968-9). Most of the identified peptides are issued from non-coding genomic regions. Some of these potential tumor antigens are present in several patients and can induce immune responses in vitro or in mouse models. There is however no evidence so far, for T cells, specific for shared tumor specific neoantigens originating from the non-coding genome in cancer patients. Indeed, identification of such tumor neoantigens would be of interest and might improve the development of cancer therapy in particular in the case of vaccination and adoptive cell therapy.

A large fraction of the non-coding genome is composed of transposable elements (TEs). TEs include 3 main classes of retrotransposons (short interspersed nuclear elements -SINE, long interspersed nuclear elements -LINE and long terminal repeats -LTRs), and DNA transposons (Grundy et al., FEBS J, 2021, doi:10.1111/febs.15722; Burns, K. H., Nat Rev Cancer, 2017, 17, 415-424; Bourque et al., Genome Biol, 2018, 19, 199, doi:10.1186/s13059-018-1577-z). Retro-transposition requires the transcription of the TEs, their reverse transcription into DNA and their integration at a different genomic position. Retro-transposition can compromise the stability of the genome, and mammalian cells protect themselves through epigenetic repression of TE transcription in adult tissues. As a result, TE transcription is relatively low (but detectable) in most adult cells, and more active during embryonic development, in stem cells and in tumors. TE de-repression in tumors occurs through multiple epigenetic changes to TE loci, including in DNA and histone de-methylation. Both epigenetic changes are related to oncogenic processes, which involve different levels of epigenetic de-regulation.

However, whether de-repressed TEs in tumors can be a source of truly tumor-specific antigens has never been questioned.

Glioblastoma (GBM) is still one of the most challenging cases in clinical oncology. The gold standard management of GBM, tumor resection followed by radiotherapy and chemotherapy (typically temozolomide), is limited in efficacy due to high rates of recurrence, overall resistance to therapy, and devastating side effects.

Thus, identification of shared tumor specific neoantigens would be of interest and might improve the development of cancer therapy in particular in the case of vaccination and adoptive cell therapy and would therefore represent a tremendous hope for treatment of glioblastoma in patients.

### SUMMARY

The present disclosure relates to a method for identifying or screening a tumor cell TE signature comprising the steps of:
i. obtaining the single cell transcriptomic TE pattern of at least one tumor cell and the single cell TE transcriptomic pattern of at least one normal cell, and
ii. performing differential expression analysis of the TE transcriptomic pattern from said at least one tumor cell with respect to said at least one normal cell, and
iii. selecting the TE transcript sequences which are differentially expressed in said at least one tumor cell as compared to said at least one normal cell thereby obtaining a tumor cell TE signature.

Typically, at step i) the single cell transcriptomic TE pattern is obtained by mapping the single-cell transcriptome to individual genomic TE occurrence.

The present disclosure also relates to a method for identifying TE-derived tumor neoantigenic peptides, the method comprising the steps of:
a) obtaining a tumor cell TE signature according to the method for identifying a tumor cell TE signature of the present disclosure, and
b) in silico translating the TE transcript sequences from the tumor cell TE signature obtained at step a) to obtain TE-derived tumor peptides.

Typically, the method for identifying TE-derived tumor neoantigenic peptides further comprises a step c) of identifying the TE derived peptides that bind at least one MHC molecule; in some embodiments, a library comprising the TE-derived peptide sequences identified at step b) is searched in the MHC ligandome from tumor cells and wherein matched peptides from the said MHC ligandome are selected, thus identifying MHC bound TE-derived peptides; in some embodiments, the TE-derived MHC bound peptides are further filtered against canonical proteins.

Typically, the method for identifying TE-derived tumor neoantigenic peptides further comprises a step d) of selecting non-redundant TE-derived peptides; in some embodiments, this step is achieved by mapping the TE-derived peptides of step c) to the individual TE genomic location and selecting uniquely mapped TE.

In some embodiments of the method for identifying TE-derived tumor neoantigenic peptides, the TE-encoded peptides which binds at least one MHC class I or II molecule of a subject with a K_{D} binding affinity of less than 10⁻⁵ M are selected.

The present disclosure further encompasses an isolated tumor neoantigenic peptide sequence having at least 8 amino acids, wherein said neoantigenic peptide comprises a TE encoded sequence and binds at least one MHC class I or II molecule of a subject with a K_{D} binding affinity of less than 10⁻⁵ M.

Said neoantigenic peptide has typically one or more of the following properties:
the TE expression is derepressed in a tumor cell as compared to non-tumor cells;
the peptide is encoded by a TE transcript sequence or a fragment thereof obtained according to the method for identifying a tumor cell TE signature as above defined;
the peptide is obtained in a method according to the method for identifying TE-derived tumor neoantigenic peptides; and/or
the peptide is encoded by a TE transcript or a fragment thereof of any one of SEQ ID NO:381 to 5020; preferably the peptide is encoded by a TE transcript or a fragment thereof of any one of SEQ ID NO: 381 to 430 and 432 to 5020; more preferably the peptide is encoded by a TE transcript or a fragment thereof of any one of SEQ ID NO: 381 to 393; 395 to 430 and 432 to 5020;
   optionally the peptide comprises at least 8 amino acids, in particular 8 or 9 to 15 amino acids, notably 12 to 15 amino acids and binds at least one MHC class I molecule of a subject or comprises from 13 to 25 amino acids and binds at least one MHC class II of a subject.

In some embodiments, the neoantigenic peptide comprises or consist of any one of SEQ ID NO: 1 to 380 or a fragment thereof, optionally the peptide is encoded by a single genomic TE. In some preferred embodiments, the neoantigenic peptide comprises or consist of any one of SEQ ID NO: 1 to 26 and 28 to 380 or a fragment thereof; preferably the neoantigenic peptide comprises or consist of any one of SEQ ID NO: 1 to 10; 12 to 26; 28 to 57; 59 to 242; 244 to 255; 257 to 319 and 321 to 380 or a fragment thereof; more preferably the neoantigenic peptide is encoded by a single genomic TE.

In some embodiments of the present disclosure, the tumor is glioblastoma tumor.

Typically, the TE is characterized by one or more of the following properties:
the TE is selected from TE over 50.10⁶ years; optionally wherein the TE is selected from the LINE-1, SVA and ERVK TE subfamilies; optionally wherein the TE is selected from L1PA/B/x TEs;
the TE is selected from TEs over 50.10⁶ years;
the TE is selected from TEs bearing an intact or nearly intact ORF;
the TE is selected from intronic or intergenic TEs
the TE is encoded by chromosome 7.

The present disclosure also encompasses a population of autologous dendritic cells or antigen presenting cells that have been pulsed with one or more of the TE-derived tumor neoantigenic peptides as above defined or transfected with a polynucleotide encoding one or more of the said peptides.

The present disclosure also encompasses a vaccine or immunogenic composition capable of rising a specific T-cell response comprising:
one or more neoantigenic peptides as above defined;
one or more polynucleotides encoding a neoantigenic peptide as above defined, optionally a neoantigenic peptide linked to a heterologous regulatory control nucleotide sequence; and/or
a population of antigen presenting cells, as above defined.

The present disclosure also encompasses an antibody, or an antigen-binding fragment thereof, a T cell receptor (TCR), or a chimeric antigen receptor (CAR) that specifically binds a neoantigenic peptide as above, optionally in association with an MHC molecule, with a K_{d}affinity of about 10⁻⁶ M or less;
optionally the antibody is a multispecific antibody that further targets at least an immune cell antigen, optionally the immune cell is a T cell, a NK cell or a dendritic cell, optionally wherein the targeted antigen is CD3, CD16, CD30 or a TCR; and/or
optionally the antibody is a multispecific antibody that further targets at least an immune cell antigen, optionally wherein the immune cell is a T cell, a NK cell or a dendritic cell, optionally wherein the targeted antigen is CD3, CD16, CD30 or a TCR.

In some embodiments, the T cell receptor as previously defined is made soluble and fused to an antibody fragment directed to a T cell antigen, optionally the targeted antigen is CD3 or CD16.

The present disclosure also encompasses a polynucleotide encoding the neoantigenic peptide as herein defined, or the antibody, the CAR or the TCR as herein defined. The present disclosure also encompasses a vector comprising said polynucleotide.

The present disclosure also encompasses an immune cell that specifically binds to one or more neoantigenic peptides as defined herein; optionally the immune cell is an allogenic or autologous cell selected from T cell, NK cell, CD4+/CD8+, TILs/tumor derived CD8 T cells, central memory CD8+ T cells, Treg, MAIT, and Yδ T cell.

The present disclosure also encompasses a T cell as defined above, which comprises:
a T cell receptor that specifically binds one or more neoantigenic peptides as defined herein, and/or
a TCR or a CAR of the present disclosure.

The present disclosure also encompasses the neoantigenic peptide, the population of dendritic cells, the vaccine or immunogenic composition, the antibody, the antigen-binding fragment thereof, the CAR, the TCR, the polynucleotide the vector, or the immune cell as defined herein for use in the treatment of cancer; optionally for inhibiting cancer cell proliferation, or for use in cancer vaccination therapy of a subject; optionally the cancer is glioblastoma.

### FIGURES

FIG. 1. Single cell TE expression distinguishes all cell populations in tumors (A) Workflow showing the strategy of alignment and TE quantification using uniquely or multiple mapped reads. (B) t-Distributed Stochastic Neighbor Embedding (tSNE) visualizing all single cells after filtering (n=3,167). Cells clusters are indicated as distinct sorted cell populations and identified based on gene expression (left), TE subfamilies expression (middle) and individual TE copies expression (right). (C) Violin plots representing the TE specific signatures for neoplastic cells (top) and immune cells (bottom). (D) Plot showing TE subfamily enrichment analysis using all expressed TE (left), neoplastic (middle) and immune (right) signatures. On x-axis is represented the ‘Adjusted p-value’ as −log 10 (adjusted p-value) using the Benjamini-Hochberg procedure. Ratio proportions (proportion in subset versus genomic proportion from RepeatMasker) are represented by proportional circles. dashes represent adjusted P-value <0.05 on x-axis. The length of the colored lines indicates the adjusted p-value. The subfamilies are colored by classes. The longer the colored line is, the smaller is the adjusted p-value. (E) Radar plots displaying the rate of genes (top) and TE (bottom) along all chromosomes. Genomic ratio based on RepeatMasker (in black) and ratio from neoplastic signature (in darkgray) are shown. (F) Barplots showing the rate of genes (first line) or TEs (second line) located in chromosome 10 (left) or 7 (right) on different subsets of features: All annotated features in the genome (Genomic), all expressed features in the datasets after filtering (Expressed), all differentially expressed features from neoplastic, immune and OPC cell populations.

FIG. 2. TE expression in neoplastic cells is enriched in elements independent of their closest gene (A) Barplot showing the distribution of different types of genomic regions for individual TE copies using RepeatMasker (4496056), all expressed TEs (130028), TEs from the neoplastic (3428) and immune signatures (2920). (B) Barplot showing the number of TEs in proximal or distal regions of closest protein-coding genes in neoplastic and immune signatures. (C) Plot showing the distance to closest protein-coding gene per class of TEs for proximal (first line) and distal (second line) TEs comparing neoplastic and immune signatures. (D) Plots summarizing the association between TEs and genes described in FIG. 1B in neoplastic (top) and immune signatures (bottom). The TE⁺gene⁺ category represents a positive correlation when the TE and gene are differentially expressed for the same cell population. The TE*gene category represents a negative correlation when the TE is differentially expressed and not the gene. The categories are also separated according to proximal and distal status.

FIG. 3. Single cell neoplastic TE signature is highly enriched in GBM cohort from TCGA compared to GTEx normal tissues. (A-B) PCA and Uniform Manifold Projections (UMAP) projection of GBM TCGA cohort, GTEx normal brain and other GTEx tissues based on single cell neoplastic TE signature, color-coded by dataset types. (C) Gene Set Enrichment Analysis (GSEA) was performed to determine the specific enrichment in neoplastic signature in GBM tumor samples and GTEx normal brain samples. Normalized Enrichment Score (NES) and FDR are indicated in the figure. (D) Violin plots showing the mean expression of single cell neoplastic signature in GBM TCGA cohort, GTEx normal brain and other GTEx datasets. (E) Violin plots showing specific expression of individual TEs in tumor samples (bulk RNA-seq analysis, top) and neoplastic cells (single cell analysis, bottom).

FIG. 4. Neoplastic-enriched TE-derived peptides are presented on HLA-I molecules and immunogenic. (A) Workflow for the identification of TE-derived peptides using mass spectrometry-based immunopeptidomics. (B) Boxplot showing the peptide-spectrum identification score (SEQUEST score) from annotated and TE-derived peptides. (C) Binding to HLA-A02*01 and HLA-B*07:01 measured as percentage of peptide-HLA-I-complex formation compared to positive control. (D) Total frequency of multimer positive populations for HLA-A*02:01 predicted or MS-derived peptides and HLA-B*07:02 MS-derived peptides in each evaluated donor. Total Frequencies are calculated considering total number of multimer positive cells in all replicates among all CD8+ T cells evaluated per donor. Lines below indicates mix of peptides used for each donor. P #: predicted TE-derived peptides; pMS #: MHC-I peptidome-derived peptides; Melan-A mutated sequenced and N #: normal proteome-derived peptides.

FIG. 5. TE derived peptides are in long ORFs starting with canonical and non-canonical start codon. Barplots showing for different subsets the quantification of LINE and LTR TEs with an intact ORF documented in gEVE database.

FIG. 6. TE-derived peptides redundancy depends on TE age. Plot showing TE family enrichment analysis using TEs coding for peptides with all assignments (left) or single assignment (right). On x-axis is represented the ‘log 2 proportion ratio’ (proportion in subset versus proportion in RepeatMasker). The significance of hypergeometric test is represented by proportion circles. The bigger circle is, the smaller is the adjusted pvalue (−log 10 adjusted pvalue).

FIG. 7. TE-derived peptides are overexpressed in GBM tumor samples. The log 2 ratio between GBM and GTEX TE-derived peptides total RNA related expression has been determined. Age information, redundancy and TE classes are considered. The tissues from GTEx are classified into 5 normal tissues categories defined in Bradley et al (Nat Commun, 2020, 11, 5332). TEs are ordered using hierarchical clustering and two groups, group 1 and group 2. Plot showing median age of TEs coding for peptides for each group.

### DETAILED DISCLOSURE

The inventors used single cell transcriptomics (scRNAseq) of tumor sample to identify pattern of individual TEs selectively expressed in tumor cells, in particular in total glioblastoma (GBM) tumor cells. They further demonstrated that peptides encoded by these selectively expressed TE are not only presented by HLA-I molecules in cancer cells and immunogenic but are also shared among patients. They also demonstrated that single-TE (non-redundant TE) encoded peptides are more tumor-specific.

Their results also show that the TEs differentially expressed in GBM tumors present a bias for TEs encoded on chromosome 7, which is fully consistent with the known recurrent amplification of this chromosome in GBM cancers. TE-derived peptides presented by MHC-I are enriched for peptides derived from specific subfamilies, including young LINE-1 and SVA elements.

Thus, the results included therein demonstrate that scRNAseq-guided, TE-centered, proteogenomics represents a powerful tool to identify tumor-specific antigens, and that TE-derived peptides recurrently presented on HLA-I molecules on GBM tumor cells are mainly encoded by young LINE-1 elements that are selectively de-repressed in such GBM tumor cells.

Because the peptides identified according to the method as herein disclosed are immunogenic in healthy patients and presented to HLA-I, they represent a source of share tumor specific neoantigens that can be used for the production of various cancer therapies including antigen presenting cells and immunogenic compositions notably for personalized vaccination strategies, but also to build CAR or TCR and produce modified immune cells comprising thereof, or to generate antibodies usable in the treatment of cancer. Identification of true specific epitopes express in many cancer patients would allow to follow these therapeutic approaches more efficiently and to strongly lower the costs. In the case of TCR adoptive therapies, identifying TCRs specific for the shared neo-epitopes would allow the development of better autologous or even allogeneic cellular therapies. It would also be possible to develop antibodies specific to the presented shared HLA-peptide complexes for ADC or CAR-T cell approaches.

### Definitions

According to the present disclosure, the term “normal” refers to the healthy state or the conditions in a healthy subject, tissue, or cell, i.e., non-pathological conditions, wherein “healthy” preferably means non-cancerous. Typically, in some embodiments, healthy cell means “non tumor cell” or “non-malignant cell”.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Malignant tumor is essentially synonymous with cancer. Malignancy, malignant neoplasm, and malignant tumor are essentially synonymous with cancer.

As used herein, the term “tumor” or “tumor disease” refers to an abnormal growth of cells (called herein neoplastic cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize).

Neoplasm is an abnormal mass of tissue as a result of neoplasia. Neoplasia (new growth in Greek) is the abnormal proliferation of cells. The growth of the cells exceeds and is uncoordinated with that of the normal tissues around it. The growth persists in the same excessive manner even after cessation of the stimuli. It usually causes a lump or tumor. Neoplasms may be benign, pre-malignant or malignant.

Cancer or tumor may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus. The tumors or cancers types as per the present disclosure also include leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, oesophagus cancer, colorectal cancer, pancreas cancer, ear, nose and throat (ENT) cancer, breast cancer, prostate cancer, cancer of the uterus, ovarian cancer and lung cancer and the metastases thereof. In some embodiments, the cancer or tumor is associated with de-repressed TEs (see notably for reference Kong, Y., Rose, C. M., Cass, A. A. et al. Transposable element expression in tumors is associated with immune infiltration and increased antigenicity. Nat Commun 10, 5228 (2019)). In some embodiments, the tumor or cancer is selected from stomach, bladder, liver, and head and neck tumors. In particular embodiments, the tumor is glioblastoma

“Growth of a tumor” or “tumor growth” according to the present disclosure relates to the tendency of a tumor to increase its size and/or to the tendency of tumor cells to proliferate.

For purposes of the present disclosure, the terms “cancer” and “cancer disease” are used interchangeably with the term “tumor” or “tumor disease”.

Cancers are classified by the type of cell that resembles the tumor and, therefore, the tissue presumed to be the origin of the tumor. These are the histology and the location, respectively.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e., a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the present disclosure, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

By “being at risk” is meant a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

The therapeutically active agents or product, vaccines and compositions described herein may be administered via any conventional route, including by injection or infusion.

The agents described herein are administered in effective amounts. An “effective amount” refers to the amount which achieves a desired reaction or a desired effect alone, together with further doses, or together with further therapeutic agents. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an agent described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on several of such parameters. In the case that a reaction in a patient is insufficient with an initial dose, higher doses (or effectively higher doses achieved by a different, more localized route of administration) may be used.

The pharmaceutical compositions as herein described are preferably sterile and contain an effective amount of the therapeutically active substance to generate the desired reaction or the desired effect.

The pharmaceutical compositions as herein described are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g., CpG oligonucleotides, cytokines, chemokines, saponin, GM-CSF and/or RNA and, where appropriate, other therapeutically active compounds. When used in medicine, the salts should be pharmaceutically compatible.

A “transposable element (TE, transposon, or jumping gene)” as used herein is a repeated DNA sequence that is able to move from one location to another in the genome either through an RNA copy generated by a reverse transcriptase (Class I TEs, retrotransposons), or by excising themselves from their original location (Class II TEs, or DNA transposons).

Retrotransposons are by far more abundant and their characteristics are similar to retroviruses, such as HIV. Retrotransposons function via reverse transcription of an RNA intermediate replicative mechanism. They are commonly grouped into three main orders: retrotransposons with long terminal repeats (LTRs) flanking the retroelement main body, which encode reverse transcriptase, similar to retroviruses; retroposons with long interspersed nuclear elements (LINEs, LINE-1s, or L1s), which encode reverse transcriptase but lack LTRs, and are transcribed by RNA polymerase II; and retrotransposons with short interspersed nuclear elements (SINEs) that do not encode reverse transcriptase and are transcribed by RNA polymerase III. DNA transposons have a transposition mechanism that do not involve an RNA intermediate. The transpositions are catalyzed by several transposase enzymes. LTRs include endogenous retroviruses (ERVs), while non-LTR TEs subdivide into long-interspersed (LINEs) and short interspersed elements (SINEs), nonautonomous transposons mobilized by the LINE integration machinery. These lineages are composed of phylogenetically related families, further branching out into multiple subfamilies, each originating from one precursor copy. With time, the accumulation of mutations introduced divergence in the consensus sequence within members of each subfamily. For review on TE retrotransposon, see Richardson, Sandra R et al. “The Influence of LINE-1 and SINE Retrotransposons on Mammalian Genomes.” Microbiology spectrum vol. 3, 2 (2015): MDNA3-0061-2014.

A typical L1 element is approximately 6,000 base pairs (bp) long and consists of two non-overlapping open reading frames (ORF) which are flanked by untranslated regions (UTR) and target site duplications. LINE-1 retrotransposons have been amplifying in mammalian genomes for greater than 160 million years. In humans, the vast majority of LINE-1 sequences have amplified since the divergence of the ancestral mouse and human lineages approximately 65-75 million years ago. Sequence comparisons between individual genomic LINE-1 sequences and a consensus sequence derived from modern, active LINE-1s can be used to estimate the age of genomic LINE-1s (Khan H, Smit A, Boissinot S; Genome Res. 2006 January; 16(1):78-87). L1 subfamilies typically categorize into old (LIM, AluJ), intermediate (LIP, L1PB, AluS), young (L1HS, L1PA, AluY) and related (HAL, FAM) subfamilies. In humans, the only autonomously active family is the long-interspersed element-1 (LINE-1 or L1), however a few L1 copies are still retrotransposition competent, all of them belonging to the youngest human-specific L1HS subfamily.

SVA elements comprise an evolutionarily young, non-autonomous retrotransposon family that arose in primate lineages approximately 25 million years ago (Hancks D C, Kazazian H H Jr, Semin Cancer Biol. 2010 August; 20(4):234-45). A typical SVA element is approximately 2,000 bp and has a composite structure that consists of: 1) a hexameric CCCTCT repeat; 2) an inverted Alu-like element repeat; 3) a set of GC-rich variable nucleotide tandem repeats (VNTRs); 4) a SINE-R sequence that shares homology with HERVK-10, an inactive LTR retrotransposon; and 5) a canonical cleavage polyadenylation specificity factor (CPSF) binding site that is followed by a poly (A) tract. The youngest SVA subfamilies include SVA-D, SVA-E, SVA-F, and SVA-F1 subfamilies.

Transposition can also be classified as either “autonomous” or “non-autonomous” in both Class I and Class II TEs. Autonomous TEs can move by themselves, whereas non-autonomous TEs require the presence of another TE to move. the TE evolutionary age can be estimated from the degeneration of their characteristic motifs as illustrated in Choudhary, Mayank Nk et al. Genome biology vol. 21, 1 16. 24 Jan. 2020. More particularly, the TE's evolutionary age can be estimated by dividing the percent divergence of extant copies from the consensus sequence by the species neutral substitution rate (i.e.: in humans: 2.2×10⁻⁹). Jukes-Cantor and Kimura distances can be calculated by aligning each TE to its consensus sequence and counting all possible mutations. Single nucleotide substitution counts were normalized by the length of the genomic TE minus the number of insertions (gaps in the consensus). These mutation rates were then used to calculate the Jukes-Cantor and Kimura distances for each genomic TE. For most of the TE subfamilies, the consensus sequences can be retrieved from the RepBase library. Full-length LINE consensus can be reconstructed as detailed in Choudhary et al. 2020.

Intact open reading frame (ORF) locations can be retrieved from gEVE database. Intact ORFs and individual TEs coordinates are typically matched to assign an intact ORF to individual TEs in case of coordinates overlap. 30517 individual TEs overlapped an intact ORF with most of them being L1 (mostly L1PA/B/x) and ERV (mostly ERV1, ERVK, ERVL) elements. To identify amino acid sequence similarity between canonical TE proteins from gEVE database and peptides from immunopeptidomics results, a blastp can typically be performed between gEVE protein sequences and the immunopeptidomics sequences. No threshold on Evalue is typically set and similarity is typically estimated and classified in 3 categories: (1) 100% match: no mismatch, no gap and query coverage per HSP to 100%; (2) At most 1 mismatch: 1 mismatch, no gap and query coverage per HSP above 85%; (3) At most 2 mismatches: 2 mismatches, no gap and query coverage per HSP above 85%.

A “representative genome” (also known as reference genome or assembly) is a digital nucleic acid sequence database, assembled by scientists as a representative example of species set of genes. As they are often assembled from the sequencing of DNA from a number of donors, reference genomes do not accurately represent the set of genes of any single individual (animal or person). Instead a reference provides a haploid mosaic of different DNA sequences from each donor.

An exon is any part of a gene that will encode a part of the final mature RNA produced by that gene after introns have been removed by RNA splicing. The term exon refers to both the DNA sequence within a gene and to the corresponding sequence in RNA transcripts.

A “messenger RNA (mRNA)” is a single-stranded RNA molecule that corresponds to the genetic sequence of a gene and is read by the ribosome in the process of producing a protein. mRNA is created during the process of transcription, where the enzyme RNA polymerase converts genes into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, and, utilizing amino acids carried by transfer RNA (tRNA), the ribosome creates the peptide sequence a process called translation.

A “transcript” as herein intended is a messenger RNA (or mRNA) or a part of a mRNA which is expressed by an organism, notably in a particular tissue or even in a particular tissue. Expression of a transcript varies depending on many factors. In particular, expression of a transcript may be modified in a cancer cell as compared to a normal healthy cell. In the present disclosure a transcript can be provided in the form of its corresponding genomic sequence.

A “transcriptome” as herein intended is the full range of messenger RNA, or mRNA, molecules expressed by an organism. In some embodiments, the term “transcriptome” or “transcriptomic pattern” can also be used to describe the array of mRNA transcripts produced in a particular cell or tissue type. In contrast with the genome, which is characterized by its stability, the transcriptome actively changes. In fact, an organism's transcriptome varies depending on many factors, including stage of development and environmental conditions. Typically, also, the transcriptome is modified in a cancer cell as compared to a corresponding (i.e.: the same type of cell typically from the same species) normal healthy cell. Typically, the transcriptome as herein intended is the human transcriptome. The terms “transcriptomic pattern” and “transcriptome” are used herein as synonyms when referred to a single cell.

A reading frame (RF) is a way of dividing the sequence of nucleotides in a nucleic acid (DNA or RNA) molecule into a set of consecutive, non-overlapping triplets.

An open reading frame (ORF) is the part of a reading frame that can be translated into a peptide. An ORF is a continuous stretch of codons that contain a start codon (for example AUG) after the transcription starting site (TSS) and a stop codon (for example UAA, UAG or UGA). An ATG codon within the ORF (not necessarily the first) may indicate where translation starts. The transcription termination site is located after the ORF, beyond the translation stop codon. In eukaryotic genes with multiple exons, ORFs span intron/exon regions, which may be spliced together after transcription of the ORF to yield the final mRNA for protein translation.

A “canonical ORF” as herein intended is a protein coding sequence with specified reading frame within a mRNA sequence, which is described or annotated in databases such as for example Ensembl genome/transcriptome/proteome database collection (typically hg19). Typically, a canonical ORF is the annotated (in reference databases) ORF of a given exon in normal healthy cells.

A “non annotated or non-canonical transcript or mRNA” as herein intended is a protein coding sequence with specified reading frame within a mRNA sequence which is not described (i.e.: unannotated) in genome databases such as for example in Ensembl genome/transcriptome/proteome database. The term “canonical protein” as herein intended refers a protein which is encoded by a canonical or annotated reading frame. In some embodiments, some non-annotated mRNA sequences may represent minor mRNA that are expressed in normal healthy cells to a level below 5%, notably below 2%, below 1%, below 0.5%, below 0.2%, or below 0.1% of the total cell mRNA.

RNA-Seq (named as an abbreviation of RNA sequencing) is a sequencing technique which uses next-generation sequencing (NGS) to reveal the presence and quantity of RNA (typically messenger RNA, mRNA) in a biological sample and generates an enormous numbers of raw sequencing reads (typically at least in the tens of millions). Single-cell RNA sequencing (scRNA-Seq) provides the expression profiles of an individual cell. A read refers to an RNA sequence from one RNA fragment from a biological sample or a single cell. The RNA sample that was sequenced is called the RNA library. RNA sequencing data are thus typically called RNA reads. There are two main ways of measuring the expression of a transcript, notably in the present case of a TE transcript, in RNA-seq data:

Counts are simply the number of reads overlapping a given genomic location.

“TPM” (“transcripts per million”) and FPKM (fragments per kilobase of exon model per million reads mapped) are also common units reported to estimate gene expression based on RNA-seq data. Both units are calculated from the number of reads that mapped to each particular gene sequence and both units are calculated taking into account two important factors in RNA-seq:
The number of reads from a gene depends on its length. One expects more reads to be produced from longer genes.
The number of reads from a gene depends on the sequencing depth that is the total number of reads you sequenced. One expects more reads to be produced from the sample that has been sequenced to a greater depth.

FPKM (introduced by Trapnell, C., Williams, B., Pertea, G. et al. Nat Biotechnol 28, 511-515 (2010).) are calculated with the following formula:

$${FPKM}_{i} = \frac{{q}_{i}}{\frac{{l}_{i}}{{10}^{3}} * \frac{{\sum }_{j} {q}_{j}}{{10}^{6}}} = \frac{{q}_{i}}{{l}_{i} * {\sum }_{j} {q}_{j}} * {10}^{9}$$

where qᵢ are raw counts (number of reads that mapped for each gene), li is gene length and total number mapped reads is the total number of mapped reads. The interpretation of FPKM is that if you sequence your RNA sample again, you expect to see for gene i, FPKMi reads divided by gene i length over a thousand and divided by the total number of reads mapped over a million.

L1 and Dewey, 2011 (Li, B., Dewey, C. N. RSEM: accurate transcript quantification from RNA-Seq data with or without a reference genome. BMC Bioinformatics 12, 323 (2011)) introduced the unit TPM and Pachter, 2011 (arXiv:1104.3889 [q-bio.GN]“Models for transcript quantification from RNA-Seq”) established the relationship between both units. It is possible to compute TPM from FPKM as follows:

$${TPM}_{t} = ( \frac{{FPFM}_{i}}{{\sum }_{j} {FPFM}_{j}} ) * 1 {0}^{6}$$

For TPM definition, the following definition can also be consulted: Wagner et al., Theory Biosci. 2012 December; 131(4):281-5.

For example, in the EMBL expression atlas database (which contains thousands of selected microarray and RNA-sequencing data that are manually curated and annotated with ontology terms Baseline expression results), baseline expression levels are set as follow and represented in different colors (see https://www.ebi.ac.uk/gxa/FAO.html):
Grey box: expression level is below cutoff (0.5 FPKM or 0.5 TPM)
Light blue box: expression level is low (between 0.5 to 10 FPKM or 0.5 to 10 TPM)
Medium blue box: expression level is medium (between 11 to 1000 FPKM or 11 to 1000 TPM)
Dark blue box: expression level is high (more than 1000 FPKM or more than 1000 TPM)

If not otherwise specified, the above-mentioned reference expression levels can be used as reference, or thresholds, in the methods and definitions of the present disclosure. In some embodiments however, other threshold values can be used. For example, depending on the mean expression of the transcript in a sample, or a cell, from the disease of interest, typically a cancer cell, the expression threshold or cut-off can be set at 7.5 TPM or 10 TPM.

The “Fold change” is a measure describing how much a quantity changes between an original and a subsequent measurement. It is defined as the ratio between the two quantities and is typically used for measuring change in the expression level of a gene or in the present case of a TE in a tumor cell as compared to a non-tumor cell. Log-ratios are often used for analysis and visualization of fold changes. The logarithm to base 2 is most commonly used.

The term “peptide or polypeptide,” is used interchangeably with “neoantigenic peptide or polypeptide” in the present specification to designate a series of residues, typically L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

“Tumor neoantigenic peptides” as per the present application are peptides that once presented by specific MHC alleles can be recognized by T cells and may induce T cell reactivity. Typically, neoantigenic peptides-specific T cells possess functional avidity that may reach the avidity strength of anti-viral T cells (see: Lennerz V et al., Cancer immunotherapy based on mutation-specific CD4⁺ T cells in human melanoma. Nat Med 2015; 21:81-5).

In some embodiments, the neoantigenic peptides are entirely absent (e.g., not detectably expressed) from the normal peptidome (in particular from the human peptidome such as for example represented in the UNIPROT database and/or from a healthy cell). Typically, tumor specific neoantigenic peptides are not detectably expressed in a normal healthy cell, or sample, and are named herein “tumor specific”.

The expression “specifically expressed” in a tumor cell type with reference a neoantigenic peptide or a TE transcript means according to the present disclosure that said peptide or TE transcript is statistically differentially (Wilcoxon test adjusted p value equal or lower to 0.05, notably equal or lower to 0.01) expressed, more particularly up-regulated, in a tumor cell as compared to a non-tumor cell. In some embodiment a log 2-fold change threshold of 0.25 in TE transcript expression in a tumor cell as compared to a non-tumor cell can also be used. Thus, in some embodiments, the peptide is encoded by a TE transcripts or a fragment thereof that is expressed in a tumor cell with a log 2-fold change of at least 0.25, notably at least 0.5, at least 0.75, at least 1, at least 1.25, at least 1.5, at least 1.75 or at least 2 as compared to a non-tumor cell. In some embodiments, the TE transcript is only expressed in one or more tumor cell(s) while being not significantly detected in normal non tumor cell(s) or sample(s) (such as in normal samples from the Genotype-Tissue Expression (GTEx) database).

Typically, a subject of the present application is a mammal and notably a human. Thus typically, the representative, or reference genome or transcriptome is the human genome or transcriptome.

In the present application, “MHC molecule” or “HLA molecule” refers to at least one MHC/HLA class I molecule or at least one MHC/HLA Class II molecule. MHC class I proteins form a functional receptor on most nucleated cells of the body. There are 3 major MHC class I genes in HLA: HLA-A, HLA-B, HLA-C and three minor genes HLA-E, HLA-F and HLA-G. 32-microglobulin binds with major and minor gene subunits to produce a heterodimer. MHC molecules of class I consist of a heavy chain and a light chain and can bind a peptide of about 8 to 11 amino acids, but usually 8 or 9 amino acids, if this peptide has suitable binding motifs, and presenting it to cytotoxic T-lymphocytes. The binding of the peptide is stabilized at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow the required flexibility. The peptide bound by the MHC molecules of class I usually originates from an endogenous protein antigen. As an example, the heavy chain of the MHC molecules of class I is typically an HLA-A, HLA-B or HLA-C monomer, and the light chain is β-2-microglobulin, in humans. There are 3 major and 2 minor MHC class II proteins encoded by the HLA. The genes of the class II combine to form heterodimeric (αβ) protein receptors that are typically expressed on the surface of antigen-presenting cells. The peptide bound by the MHC molecules of class II usually originates from an extracellular or exogenous protein antigen. As an example, the α-chain and the β-chain are in particular HLA-DR, HLA-DQ and HLA-DP monomers, in humans. MHC class II molecules are capable of binding a peptide of about 8 to 20 amino acids, notably from 10 to 25 amino acids or from 13 to 25 amino acids if this peptide has suitable binding motifs, and of presenting it to T-helper cells. The peptide lies in an extended conformation along the MHC II peptide-binding groove which (unlike the MHC class I peptide-binding groove) is open at both ends. It is held in place mainly by main-chain atom contacts with conserved residues that line the peptide-binding groove.

The term “peptidome” refers to the complete set of peptides expressed by a particular genome, or present within a particular organism or cell type (such as a cancer cell). Proteomic analysis (proteomics) thus refers to the separation, identification, and quantification of the entire set of peptides or proteins expressed by a genome, a cell, or a tissue at a specific point in time.

Proteomics analyses are typically based on two major techniques, namely two-dimensional gel electrophoresis (2-DGE) (Harper S et al., In: Coligan J E, Dunn B M, Speicher D W, Wing-field PT, editors. Current Protocols in Protein Science. John Wiley & Sons; Hoboken, N.J.: 1998. pp. 10.4.1-10.4.36.) and Mass Spectrometry (MS) (Aebersold & Mann, 2003), which are both powerful methods for the analysis of complex mixtures of proteins. HPLC is an alternative separation technique for proteomic studies, especially in separation and identification of low-molecular-weight proteins and peptides (Garbis et al., 2005). MS allows the determination of the molecular mass of proteins or peptides based on the mass to charge ratio (m/z) of ions in the gas phase. The terms “gel-based” or “gel-free” proteomics are used in relation to the applied separation techniques, 2-DGE or HPLC; proteomics approaches can also be “bottom-up” or “top-down,” which basically identify proteins from their protease (e.g., trypsin) digests or, as a whole, via a mass spectrometer, respectively.

Bottom-up proteomics is a common method to identify proteins from a biological sample (tissue(s) or cells) and characterize their amino acid sequences and post-translational modifications by proteolytic digestion of proteins prior to analysis by mass spectrometry. The crude protein extract is enzymatically digested, followed by one or more dimensions of separation of the peptides typically by liquid chromatography coupled to mass spectrometry, a technique known as shotgun proteomics. By comparing the masses of the proteolytic peptides or their tandem mass spectra with those predicted from a sequence database or annotated peptide spectral in a peptide spectral library, peptides can be identified, and multiple peptide identifications assembled into a protein identification.

In top-down proteomics, intact proteins are purified prior to digestion and/or fragmentation either within the mass spectrometer or by 2D electrophoresis. Top-down proteomics either uses an ion trapping mass spectrometer to store an isolated protein ion for mass measurement and tandem mass spectrometry (MS/MS) analysis or other protein purification methods such as two-dimensional gel electrophoresis in conjunction with MS/MS.

From the data generated by the MS, the protein is either sequenced de novo by manual mass analyses of the spectra or processed automatically via sequence search engines such as SEQUEST, Mascot, Phenyx, X!Tandem, and OMSSA. These algorithms are developed based on the correlation between experimental and theoretical MS/MS data; the latter being generated from in silico digestion of protein databases such as UniProt/Swiss-Prot (Deutsch, Lam, & Aebersold, 2008).

The term “immunopeptidome”, also commonly named “immunopeptidomic pattern”, “pMHC repertoire”, or “MHC-ligandome” or “HLA ligandome”, refers to the complete set of peptides within a particular cell type, which are bound to at least one MHC/HLA molecule at the cell surface. Correspondingly, “immunopeptidomics” has emerged as a term to describe analysis of the MHC/HLA-ligandome. The most common immunopeptidomics methods rely on mass spectrometry (MS). Immunopeptidomics samples are generally prepared by isolating MHCs, for example by using an allele-specific antibody, pan-specific antibody, or engineered affinity tag system, from lysed cells or tissues. Isolated complexes are acid eluted, and peptides are purified from the MHC molecules using molecular weight cut-off filtration (MWCO), solid phase extraction or other techniques, and are subsequently analyzed by MS (see for example for review L. E. Stopfer et al., Immuno-Oncology and Technology, Volume 11, 2021, 100042).

Unless specifically stated or obvious from context, as used herein, the term “about” is to be understood as within a range of normal tolerance in the art, for example within 2 standard deviations of the mean. About can be understood as within 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Method for Identifying a Tumor Cell TE-Signature

The method for identifying a tumor cell TE-signature of the present disclosure encompasses the following steps:
i. obtaining the single cell transcriptomic TE pattern of at least one tumor cell and the single cell TE transcriptomic pattern of at least one non-tumor cell, and
ii. performing differential expression analysis of the TE transcriptomic pattern from said at least one tumor cell with respect to said at least one normal cell, and
iii. selecting the TE transcript sequences which are differentially expressed in said at least one tumor cell as compared to said at least one normal cell thereby obtaining a tumor cell TE signature.

Typically, the one or more tumor cells (also named herein neoplastic cells) are from the same patient, and/or are obtained from the same tumor location and/or the same type of tumor, and/or from the sample tumor sample.

By the same type of cancer or tumor it is herein intended tumors or cancers affecting the same organs (skin, breast, lung, brain, urinary bladder, kidney, stomach, intestine, spleen, pancreas, prostate, uterine, thyroid, ovaries, endocrine glands, uterus, testes, tongue, esophagus, liver, gall, rectum, skin, etc), or the same tissue (such as carcinomas, sarcomas, myeloma, leukemias, lymphomas, etc.).

Similarly, the one or more non-tumor (e.g. “normal” or “healthy”) cells are typically obtained from the said same patient, and/or from juxta-tumor sample(s) from the same or different patient(s). The non-tumor cell can be typically tumor-infiltrating cells or cells from the juxta tumor environment. According to the present disclosure, the non-tumor cells can be from one or more types including tumor infiltrating immune cells (such as macrophages) and non-immune cells from the juxta tumor environment. For example, when the tumor is a glioblastoma, non-tumor cells from the tumor microenvironment (i.e., from juxta tumor samples) include immune cells (typically macrophages), oligodendrocytes and their precursors (OPCs), neurons, astrocytes and vascular type cells.

The transcriptomic pattern of these cells can be obtained by performing high-depth single-cell RNA sequencing (RNA-seq) (see notably Darmanis, Spyros et al. Cell reports vol. 21, 5 (2017): 1399-1410 for an example detailed proceeding). Briefly, single-cell suspensions can be analyzed (reverse transcription followed by PCR amplification) using the Smart-seq2 protocol (also detailed in Picelli S, Faridani O R, Björklund A K, Winberg G, Sagasser S, Sandberg R., Nat Protoc. 2014 January; 9(1):171-81).

In some embodiments, short reads which size is typically less than 400 base pairs (bp), notably less than 200 bp or even less than 100 bp, while being preferably at least 50 bp, notably at least 75 bp, or at least 100 bp can be used. In some other embodiments, long reads or more than 10-15 kbp can also be used. Typically, cells can be sequenced using for example 75-bp-long paired-end reads on a NextSeq instrument (Illumina) and High-Output v2 kits (Illumina).

Alternatively, public single cell (sc) RNAseq data (e.g., from the Sequence Read Archive (SRA) bioinformatic database, which is the largest publicly available repository of high throughput sequencing data) can also be used, notably when data from tumor cells and non-tumor cells from the said tumor microenvironment, and/or from cells infiltrating the said tumor are available.
Step ii) typically includes the alignment of the reads to the reference genome and the assembly of the alignments into full-length transcripts, the quantification of the expression levels of each gene and transcript, the normalization of the mapped data and the calculation of the differences in expression for all TE in tumor cells vs. non tumor cells).

Raw RNA reads can be aligned (i.e.: mapped) to the human genome (such as the human genome assembly hg19, or hg38) as detailed in the results enclosed (but see also Darmanis S et al., PNAS 2015 Jun. 9; 112(23):7285-90) using typically a software aligner such as the Spliced Transcripts Alignment to a Reference (STAR) software (Dobin, Alexander et al. Bioinformatics (Oxford, England) vol. 29, 1 (2013): 15-21).

scRNAseq reads can be mapped to transposable elements (TE) subfamilies (as done for example in Kong et al., Nat Commun 2019, 10, 5228) and/or to individual genomic TE locus or occurrence. Typically, according to the present disclosure, scRNAseq reads are mapped to individual genomic TE occurrences. Furthermore, to obtain accurate estimate expression of both the older TE subfamilies and the youngest TE subfamilies (which mapping to individual genomic location can be more especially affected by the high conservation of their repeat motifs) both multi-mapping TE reads (i.e., TE sequencing reads that map at more than one position in the genome) and uniquely mapping TE reads (that map/align at only one position in the genome) are typically considered (see notably Lanciano, S., and Cristofari, G. (2020). Measuring and interpreting transposable element expression. Nat Rev Genet 21, 721-736.).

For TE mapping, a file of annotated TE positions can be added. Thus, Transposable Elements annotations can be typically retrieved from various databases and merged if needed (as done for example in example enclosed) to obtain typically information on TE such as the Class, Family, Subfamily, Divergence, and/or coordinates. A detailed example proceeding is also described in the Example section of the present disclosure. Typically, Raw RNA reads (75 bp paired-end unstranded reads) can be mapped to the human genome sequences (hg19) using the 2—pass mode of STAR (such as the version 2.7.1.a) using the following parameters: —quantMode GeneCounts, —twopassMode Basic, —alignSJDBoverhangMin 1, —bamRemoveDuplicatesType UniqueIdentical, —winAnchorMultimapNmax 1000, —outFilterMultimapNmax 1000, —outFilterScoreMinOverLread 0.33, —outFilterMatchNminOverLread 0.33, —outFilterMismatchNoverLmax 0.04, —outMultimapperOrder Random, —sjdbOverhang 76).

In particular embodiments, TEs that are entirely included within exons are deleted from the single cell transcriptomic TE pattern. This means that the single cell transcriptomic TE pattern obtained in (step (i)) does not comprise TEs that are entirely included within exons.

Gene and TE expression can be quantified according to classical means in the field, as also exemplified in the Materials and Methods included herein. For example, to perform quantification of TE and gene expression, featureCounts from Subread (v1.6.4) can be computed on each genome-mapped reads files (well-suited methods are notably described in Teissandier, A., Servant, N., Barillot, E., and Bourc'his, D. (2019). Tools and best practices for retrotransposon analysis using high-throughput sequencing data. Mob DNA 10, 52). As a matter of example the following parameters can be used in featureCounts depending on the analysis: (1) for gene expression: —p—ignoreDup—g gene_id using gencode gtf annotation file; (2) for TEs expression on individual copies (a) with only uniquely mapping reads: —p—ignoreDup—g transcript_id using TEtranscript hg19 gtf annotation file; (b) with uniquely and multi-mapping reads: —p—ignoreDup—g transcript_id —M—primary (3) for TEs expression on subfamilies with uniquely and multi-mapping reads: —p—ignoreDup—g gene_id—M—primary. Cell count files can then be merged into a matrix using a routine python script (Python 3.6).

Exploratory analysis, visualization, and statistical modeling are also typical steps after assembling and quantifying transcripts. The R programming language and the Bioconductor software suite can typically be used according to the present disclosure and provides a set of tools ranging from plotting raw data, to normalization, to downstream statistical modeling. Indeed, the scater package is an open-source R/Bioconductor software package that implements a convenient data structure for representing scRNA-seq data and contains functions for pre-processing, quality control, normalization and visualization. It offers a workflow to convert raw read sequences into a dataset ready for higher-level analysis within the R programming environment. Scaling normalization is typically required in RNA-seq data analysis to remove biases caused by differences in sequencing depth, capture efficiency or composition effects between samples. Frequently used methods for scaling normalization include the trimmed mean of M-values (Robinson M. D. et al., Genome Biol., 2010, 11, R25.), relative log-expression (Anders S. et al., Genome Biol., 2010, 11, R106) and upper-quartile methods (Bullard J. H. et al., BMC Bioinformatics, 2010, 247, 1-62.). The scran package of scater, which implements a method utilizing cell pooling and deconvolution to compute size factors is also well suited to scRNA-seq data according to the present disclosure (Lun A. T. L. et al., Genome Biol., 2016b 17, 75). For more details, see also Davis J McCarthy, Kieran R Campbell, Aaron T L Lun, Quin F Wills, Scater: pre-processing, quality control, normalization and visualization of single-cell RNA-seq data in R, Bioinformatics, Volume 33, Issue 8, 15 Apr. 2017, Pages 1179-1186.

Optionally, considering the uniquely mapped reads TE matrix, individual TEs with less than 1 count/cell in average can be removed, while for multi-mapped reads, individual TEs with less than 5 counts in at least 20 cells can be removed to take into account expression in small populations. Low quality cells are also typically removed from the analysis. For example, low quality cells may be considered as such if they have library sizes below 100,000 reads; and/or express fewer than 5,000 genes; and/or have spike-in proportions above 10%; and/or have mitochondrial proportions above 10%.

Typically, to visualize the transcriptomic landscape across the various sequenced single cells, dimensional reduction can be used to generate a two-dimensional (2D) map. Briefly, genes with the highest over-dispersion are selected and used to construct a cell-to-cell dissimilarity matrix. Then a t-distributed stochastic neighbour embedding (tSNE) can be performed on the resulting distance matrix to create a 2D map of the cells. k-means clustering on the 2D tSNE map can further be used.

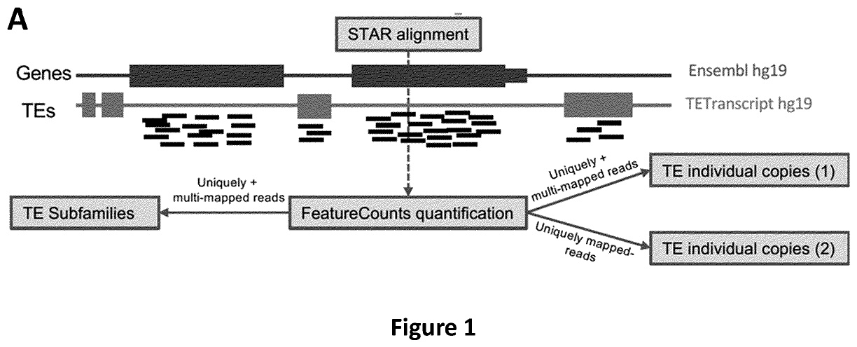

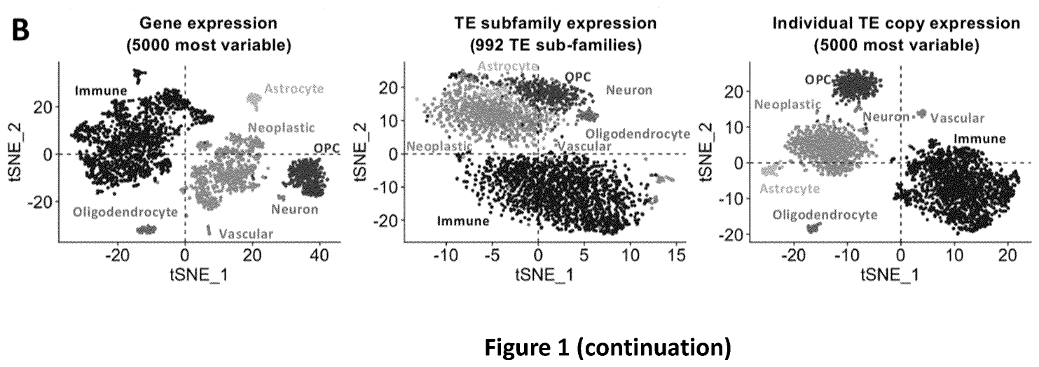

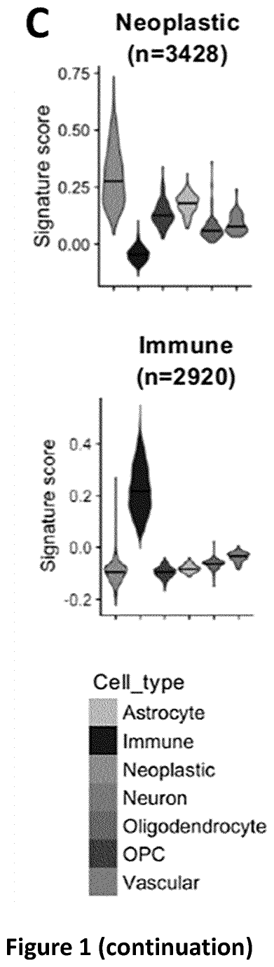

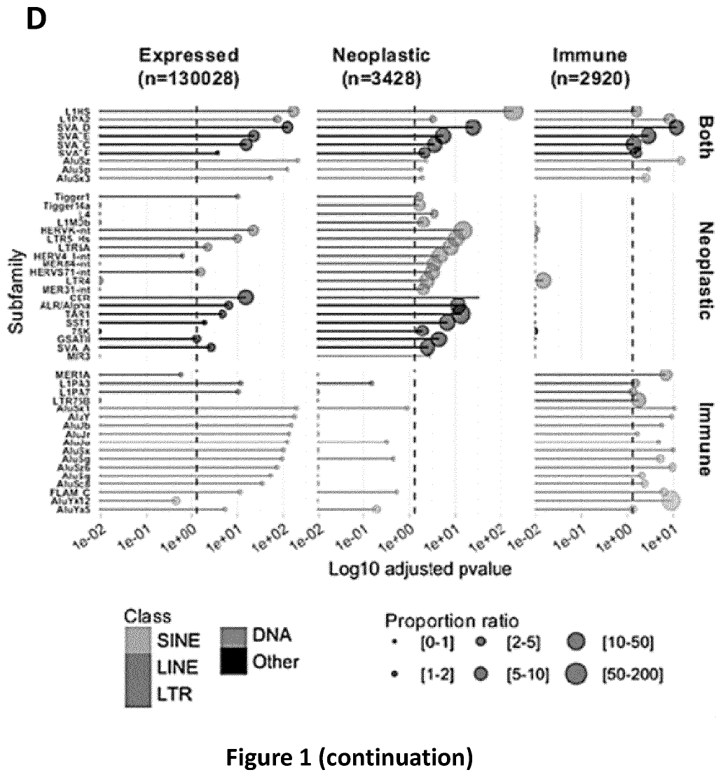

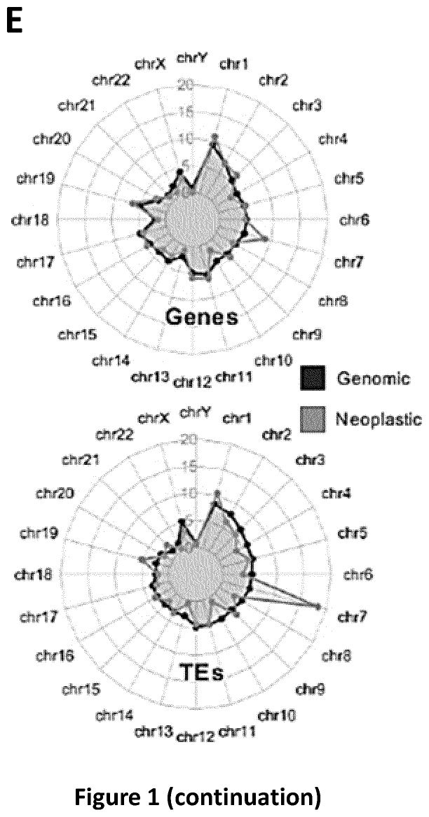

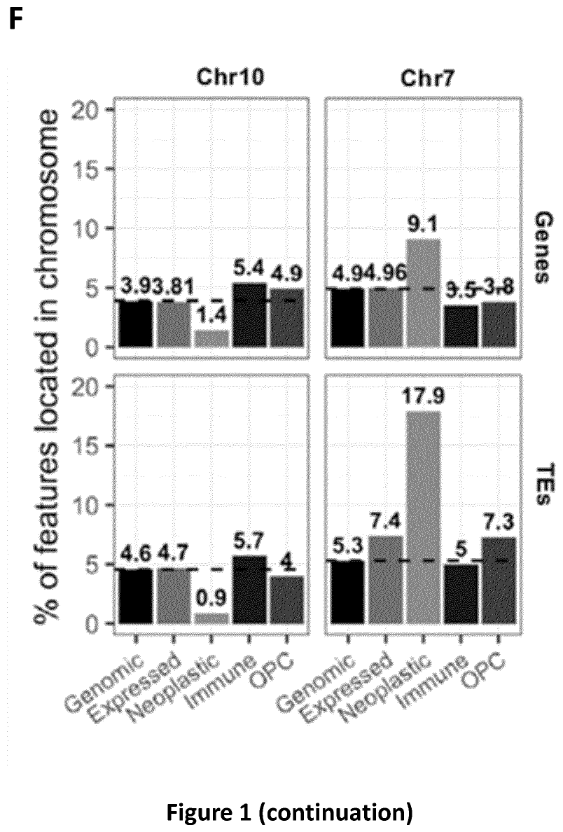

An example workflow showing the strategy of alignment based on individual TE occurrence as opposed to family level and TE quantification using uniquely or multiple mapped reads is notably illustrated in the FIG. 1A.
At step iii), the TE transcript sequences which are differentially expressed (i.e.: which expression is upregulated) in at least one tumor cell as compared to said at least one non-tumor cell are selected and a tumor cell TE signature is obtained. According to the present disclosure, TE transcripts which are statistically differentially expressed, typically with an adjusted p value equal or lower to 0.05, notably equal or lower to 0.01, in a tumor cell as compared to a non-tumor cell are selected. Alternatively, or in addition, in some embodiments, TE transcripts that are expressed with an average log 2-fold of 0.25 change in a tumor cell as compared to a non-tumor cell can be selected. In some embodiments, the peptide is encoded by a TE transcripts or a fragment thereof that is expressed in a tumor cell with a log 2-fold change of at least 0.25, notably at least 0.5, at least 0.75, at least 1, at least 1.25, at least 1.5, at least 1.75 or at least 2 as compared to a non-tumor cell. In some embodiment, the TE signature as per the present disclosure encompasses the at least 30, notably the at least 25, the at least 20, the at least 15, the at least 10, the at least 5 most differentially expressed TE for the tumor cell as compared to the at least one other non-neoplastic cell(s).

According to the present disclosure, the tumor cell Transposable Element (TE) signature corresponds to the TE transcripts which are specifically expressed by the tumor cell, in particular in some embodiments, the TE transcripts selected in the tumor cell signature are not found in the single cell transcriptomic pattern of TE transcripts obtained from the at least one non-tumor cell.

Typically, the differential analysis is performed in one or more tumor cells, from one or more tumor samples, from one or more patients against one or more non-tumor cells from one or more samples, from one or more subject. As previously mentioned, the tumor cells can be from the same or not tumor or tumor type. The non tumor cell can be or not from the same tissue sample, including immune cells, such as for example tumor infiltrating immune cells (such as macrophages) and non-tumor cells from the tumor microenvironment (e.g., from juxta tumor samples). In some embodiments, the differential analysis is performed between cells from the same patient and notably from the same sample or from samples collected from the same type of tumor (from one or more patient) and samples of the close environment of said tumor (i.e., juxta tumor samples) from one or more subject. In some embodiments, the one or more tumor cells can be obtained from tumor samples from the same patient at various time. T The method as herein disclosed allows to obtain a set of TE transcripts which are differentially expressed in a tumor cell, also named herein tumor cell “TE-signature” or tumor cell “transcriptomic TE pattern”, as compared to a non-tumor cell.

Differential analysis can be performed for example as detailed in the Materials and Methods paragraph of the Example Section.

### Method for Identifying or Screening TE-Derived Tumor Neoantigenic Peptides

The method comprises the obtention of single tumor cell TE-signature (or transcriptomic TE pattern) followed by in silico translation of the TE transcript sequences from the said tumor cell TE signature to obtain TE-derived tumor peptides.

The methods comprise a step of identifying the open reading frame (ORF) sequences from the transcripts of the TE-signature. In some embodiments, the transcripts are then in silico translated in six frame translations (both forward and reverse direction), and the resulting amino-acid sequences are then fragmented at all stop codons to obtain TE-encoded tumor peptide sequences than can be grouped to form a TE-derived tumor peptide library.

In some embodiments, the method further comprises a step allowing to identify the TE derived peptides that bind a least one MHC molecule. According to such embodiments, a library comprising the TE-encoded tumor peptide sequences (tumor TE library), obtained as above described from the TE tumor signature is typically compared to the MHC/HLA-ligandome obtained from more tumor cell(s) (including tumor cells from the same and/or different tumor types, such as for example glioblastoma cells) from one or more sample(s). Peptides from the MHC/HLA-ligandome that match with the tumor TE library are typically selected. This step allows the non-ambiguous identification of TE-encoded tumor neoantigenic peptides that are presented by HLA/MHC molecules.

Typically, the tumor TE library as above described is combined with the human protein sequences (i.e.: the human annotated proteome—e.g., Uniprot/SwissProt).

The identification of the TE derived peptides that bind a least one MHC/HLA molecule according to the present disclosure is typically achieved through a proteogenomic approach, wherein mass spectrometry (MS)-based proteomics (and notably immunoproteomics) data are matched against the peptide's library obtained from the tumor TE library as defined above more particularly, open reading frames derived from de novo assembled transcripts (e.g.: the tumor TE library previously defined) are searched against immunopeptidomics MS/MS spectra (obtained from a tissue samples or cells including cell lines such as tumor samples and tumor cells, in particular tumor samples or cell lines).

The MHC-ligandome is thus typically in the form of raw mass spectrometry (MS) data (i.e.: spectra) obtained in MS-based proteomics (notably immunoproteomics) techniques such as bottom-up proteomics (shot-gun proteomics) and top-down proteomics from one or more tissue sample or cells (e.g.: tumor samples and tumor cells).

The immunopeptidomics approach is typically based on immunoaffinity purification (IP) of HLA/MHC complexes typically from mild detergent solubilized lysates, followed by extraction of the HLA/MHC peptides (HLA/MHCp). The extracted peptides are then separated by chromatography and directly injected into a mass spectrometer. The tumor MHC/HLA-ligandome is typically obtained by first purifying surface MHC-bound (i.e., HLA-I or HLA-2 molecules) peptides followed by their amino acid sequence characterisation. Typically, the MHC/HLA ligandome is obtained from tumor cells (such as glioblastoma cells) from one or more tumor samples (e.g., biopsy or tissue) or tumor cell lines. For example, MHC/HLA-bound molecules can be purified by immunoprecipitation from the cell lysate, using an antibody specific to the desired MHC/HLA species (e.g., using MHC/HLA-IP). MHC/HLA-associated peptides can be separated from the larger MHC/HLA components and the peptide fraction can be further analysed by LC tandem mass spectrometry (LC-MS/MS).

The peptide sequences can be identified by spectral interpretation. The large-scale data acquired from high-resolution mass spectrometers are typically interpreted using algorithms that enable assignment of mass spectra to amino acid sequences There is a variety of software available to the skilled person for interpretation of MS fragment spectra (see for example Purcell, A. W., Ramarathinam, S. H. & Ternette, N. Mass spectrometry-based identification of MHC-bound peptides for immunopeptidomics. Nat Protoc 14, 1687-1707 (2019) or Prianichnikov, Nikita et al. “MaxQuant Software for Ion Mobility Enhanced Shotgun Proteomics.” Molecular & cellular proteomics: MCP vol. 19, 6 (2020): 1058-1069). MS-based immunopeptidomic analysis are also well detailed in Forlani, Greta et al. MCP, vol. 20 100032. 6 Jan. 2021; as well as Chong, Chloe et al. “High-throughput and Sensitive Immunopeptidomics Platform Reveals Profound Interferony-Mediated Remodeling of the Human Leukocyte Antigen (HLA) Ligandome.” Molecular & cellular proteomics: MCP vol. 17, 3 (2018): 533-548, which refers to the use of MaxQuant computational proteomics platform to search the peak lists against the UniProt databases—see Cox, Jiirgen, and Matthias Mann. Nature biotechnology vol. 26, 12 (2008): 1367-72.). Additional references which describe well-suited protocols for obtention of MS raw data usable according to the present disclosure are also provided in the results of the present application. According to the method of the present disclosure public MS data can be used as illustrated for example in the results included herein.

In some embodiments, the selected TE-encoded tumor neoantigenic MHC-bound peptides are further filtered against canonical proteins, typically canonical proteins from the human proteome (e.g.: typically obtained from Swiss-Prot and TrEMBL databases). UniProtKB/TrEMBL is a computer-annotated protein sequence database complementing the UniProtKB/Swiss-Prot Protein Knowledgebase. UniProtKB/TrEMBL contains the translations of all coding sequences (CDS) present in the EMBL/GenBank/DDBJ Nucleotide Sequence Databases and also protein sequences extracted from the literature or submitted to UniProtKB/Swiss-Prot. The database is enriched with automated classification and annotation.

In some embodiments, non-redundant peptides (i.e., which are encoded by a single genomic TE) are further selected. Such selection can be achieved as done for example in the results included herein by mapping the identified TE-encoded tumor neoantigenic MHC-bound peptides to the corresponding TEs in the TE signature.

In other embodiments redundant peptides are further selected. Redundant peptides with low genomic TE occurrence (encoded by e.g.: less than 100, notably less than 50, notably less than 10 genomic TE occurrences) that are encoded by a TE, which expression is highly upregulated in a tumor cell (log 2 fold change of at least 0.25, notably at least 0.5, at least 1, at least 1.5) and/or that is not expressed in a normal cell or sample (for example using the GTEx database) are of particular relevance.

Determination of the binding of putative neoantigen peptides obtained from the tumor cell TE-signature (and notably of the MHC/HLA bound peptides identified in the method described above) to at least one MHC molecule can also be performed in silico.

When carried out on human samples, the method may comprise a step of determining the patient's class I or class I Major Histocompatibility Complex (MHC, aka human leukocyte antigen (HLA) alleles).

An MHC allele database is carried out by analyzing known sequences of MHC I and MHC II and determining allelic variability for each domain. This can be typically determined in silico using appropriate software algorithms well-known in the field. Several tools have been developed to obtain HLA allele information from genome-wide sequencing data (whole-exome, whole-genome, and RNA sequencing data), including OptiType, Polysolver, PHLAT, HLAreporter, HLAforest, HLAminer, and seq2HLA (see Kiyotani K et al., Immunopharmacogenomics towards personalized cancer immunotherapy targeting neoantigens; Cancer Science 2018; 109:542-549). For example, the seq2hla tool (see Boegel S, Lower M, Schafer M, et al. HLA typing from RNA-Seq sequence reads. Genome Med. 2012; 4:102), which is well designed to perform the method as herein disclosed is an in silico method written in python and R, which takes standard RNA-Seq sequence reads in fastq format as input, uses a bowtie index (Langmead B, et al., Ultrafast and memory-efficient alignment of short DNA sequences to the human genome. Genome Biol. 2009, 10: R25-10. 1186/gb-2009-10⁻³-r25) comprising all HLA alleles and outputs the most likely HLA class I and class II genotypes (in 4 digits resolution), a p-value for each call, and the expression of each class.

The affinity of all possible peptides encoded by each transcript sequence for each MHC allele from the subject can be determined in silico using computational methods to predict peptide binding-affinity to HLA molecules. Indeed, accurate prediction approaches are based on artificial neural networks with predicted IC₅₀. For example, NetMHCpan software which has been modified from NetMHC to predict peptides binding to alleles for which no ligands have been reported, is well appropriate to implement the method as herein disclosed (Lundegaard C et al., NetMHC-3.0: accurate web accessible predictions of human, mouse and monkey MHC class I affinities for peptides of length 8-11; Nucleic Acids Res. 2008; 36:W509-W512; Nielsen M et al. NetMHCpan, a method for quantitative predictions of peptide binding to any HLA-A and -B locus protein of known sequence. PLoS One. 2007; 2:e796, but see also Kiyotani K et al., Immunopharmacogenomics towards personalized cancer immunotherapy targeting neoantigens; Cancer Science 2018; 109:542-549 and Yarchoan M et al., Nat rev. cancer 2017; 17(4):209-222, see also Reynisson B., Barra C., Kaabinejadian S., Hildebrand W. H., Peters B., Nielsen M. J. Proteome Res. 2020; 19:2304-2315 and Jurtz V, Paul S, Andreatta M, Marcatili P, Peters B, Nielsen M J Immunol. 2017 Nov. 1; 199(9):3360-3368 that discloses NetMHCpan-4.0 version.). NetMHCpan software predicts binding of peptides to any MHC molecule of known sequence using artificial neural networks (ANNs). The method is trained on a combination of more than 180,000 quantitative binding data and MS derived MHC eluted ligands. The binding affinity data covers 172 MHC molecules from human (HLA-A, B, C, E), mouse (H-2), cattle (BoLA), primates (Patr, Mamu, Gogo) and swine (SLA). The MS eluted ligand data covers 55 HLA and mouse alleles. NetMHCpan-4.0 version also pr Improved Peptide-MHC Class I Interaction Predictions Integrating Eluted Ligand and Peptide Binding Affinity Data.

In example embodiments, TE-encoded peptide from the tumor cell TE-signature as herein disclosed and having a Kd affinity of predicted peptides for MHC alleles with a score less than 10⁻⁴. 10⁻⁵, 10⁻⁶, 10⁻⁷, or less than 500 nM or a rank less than 2% (typically depending on netMHCpan version), are selected as tumor neoantigenic peptides.

MixMHCpred (v.2.0.2) (see Bassani-Sternberg M., et al., PLoS Comput. Biol. 2017; 13) and MixMHC2pred (v.1.0) (see Racle J., et al., Nat. Biotechnol. 2019; 37:1283-1286) can also be used to predict binding of peptides on patients HLA/MHC class I alleles and patients HLA/MHC class II alleles respectively as illustrated in Forlani, Greta et al. (MCP vol. 20, 2021: 100032).

In some embodiments, peptides binding to MHC Class 1 molecules can thus be predicted using for example the NetMHCpan 4.1 suite (http://www.cbs.dtu.dk/services/NetMHCpan/), using “HLA allele”=A2, “peptide length”=8-11 and “rank threshold for strong binding”=0.5% for which an example protocol is detailed in the results included in the Examples' Section.

Typically, TE-encoded peptides from the tumor cell TE signature having a predicted K_{d} affinity for MHC alleles with a score less than 50 nM or a rank less than 0.5% (typically depending on the netMHCpan version) are selected as tumor neoantigenic peptides. Thus, in some embodiments, a TE-encoded neoantigenic peptide as per the present disclosure, which typically identified as per the method, binds at least one HLA/MHC molecule with an affinity sufficient for the peptide to be presented on the surface of a cell as an antigen. Generally, the neoantigenic peptide has an IC₅₀ affinity of less than 10⁻⁴. or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less for at least one HLA/MHC molecule (lower numbers indicating greater binding affinity), typically a molecule of said subject suffering from a cancer, or a tumor.

### Neoantigenic Peptides, Polynucleotides and Vectors

The present disclosure also encompasses an isolated tumor neoantigenic peptide having at least the following characteristics:
i. it has at least 8 amino acids and comprise a TE encoded sequence.
ii. it binds at least one MHC class I or II molecule of a subject with a K_{D} binding affinity of less than 10⁻⁵ M and/or it is presented by an MHC molecule of a subject.

MHC binding of a peptide as herein disclosed can be assessed in silico as previously described. Kd affinity for at least one MHC/HLA molecule can also be determined or predicted in vitro by using tetramer preparation as illustrated in the examples. Briefly, HLA 02:01/peptide multimers can be prepared using adapted commercial kits (for example EasYmers® kits from ImmunAware® which can be used according to their training guide) and incubated with human CD8⁺ prepared from healthy donors. Tetramer-CD8⁺ cell binding can be assessed by flow cytometry. Typically, binding affinity can be determined as a percentage of binding to a positive control. Generally, peptides showing a percentage of binding of at least 30%, notably at least 40% or even at least 50% of the positive control are selected. Typically, the neoantigenic peptide as per the present disclosure, and typically obtainable as per the present method, binds at least one HLA/MHC molecule with an affinity sufficient for the peptide to be presented on the surface of a cell as an antigen. In some embodiments, the neoantigenic peptide has an IC₅₀ of less than 10⁻⁴. or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less (lower numbers indicating greater binding affinity). For example, the neoantigenic peptide has an IC₅₀ comprises between 0.1 nM and 500 nM, notably between 0.1 nM and 200 nM, notably between 1 and 200 nM. In some embodiments, a neoantigenic peptide of the present disclosure binds an MHC class I or class II molecule with a binding affinity Kd of less than about 10⁻⁴, 10⁻⁵, 10⁻⁶, 10⁻⁷, 10⁻⁸ or 10⁻⁹ M (lower numbers indicating higher binding affinity), notably comprised between 10⁻⁴ and 10⁻⁹ M, in particular between 10⁻⁴ and 10⁻⁸ M, notably comprise between 10⁻⁴ and 10⁻⁷ M.

In some embodiments, a neoantigenic peptide of the present disclosure binds an MHC class I molecule with a binding affinity of less than 2% percentile rank score predicted for example by NetMHCpan 4.0. In some embodiments, a neoantigenic peptide of the present disclosure binds an MHC class II with a binding affinity of less than 10% percentile rank score predicted for example by NetMHCpanII 3.2.

Presentation of a neoantigenic peptide according to the present disclosure, by an MHC/HLA molecule can also be assessed by interrogating the tumor immunopeptidome with the said neoantigenic peptide sequence as previously detailed.

The tumor neoantigenic peptide of the present disclosure further exhibits one or more of the following properties:
iii. The TE expression is derepressed in a tumor cell as compared to a non-tumor cell.

By derepressed in a tumor cell, it is herein intended that the expression of TE transcript sequence is statistically significantly up regulated (as previously defined) in a tumor cell as compared to a normal healthy cell. In some embodiments, the TE expression is derepressed in a tumor cell from a given type of cancer. In some embodiments, the TE transcript is expressed with an average log 2-fold of at least 0.25 change in a tumor cell, notably at least 0.5, at least 0.75, at least 1, at least 1.25, at least 1.5, at least 1.75 or at least 2 as compared to one or more non-tumor cell(s). For example, in some embodiments, the TE is derepressed in glioblastoma. Validation of TE specific tumor cell expression can be assessed as exemplified in the Example Section by performing RNAseq analysis. For example, the TE transcript sequence according to the present disclosure is overexpressed in scRNAseq from one or more tumor cell(s) as compared to scRNAseq from non-tumor cell(s) (for example including tumor infiltrating cells, notably immune cells such as macrophages) and/or in TCGA juxta-tumor bulk RNAseq samples (typically from the same tumor as the tumor cell used for the tumor single cell analysis). In some embodiments of the present disclosure the TE transcript sequence is not expressed non-tumor cell(s) (including tumor infiltrating cells), in samples from normal tissues and/or in juxta-tumor samples (obtained for example from the TCGA database).
iv. The TE is selected from TE over 50.106 years;
v. the TE is selected from the LINE-1, SVA and ERVK TE subfamilies; more particularly the TE is selected from L1PA/B/x TEs;
vi. The TE is selected from TEs bearing an intact or nearly intact ORF (no more than 2, notably no more than 1 mismatch between canonical TE protein from typically the gEVE database and the peptides sequences retrieved from immunopeptidomic profiles);
vii. The TE is selected from unique peptide-encoding TEs;
viii. The TE is selected from intronic or intergenic TEs (typically distal TEs located at more than 2 kb from the nearest gene).
ix. The TE is encoded by chromosome 7.

Typically, a neoantigenic peptide of the present disclosure is obtained according to the method as previously detailed.

In some embodiments, the tumor cell TE-signature is a glioblastoma cell TE-signature and the peptide sequences is obtained from a glioblastoma cell TE signature comprising the transcript sequences of SEQ ID NO:381 to 5020.

In some embodiments, the tumor cell TE-signature, in particular a glioblastoma cell TE-signature, excludes TEs that are entirely included with exons. In some more particularly embodiments, the neoantigenic peptide sequences is obtained from a glioblastoma cell TE signature comprising the transcript sequences of SEQ ID NO: 381 to 430 and 432 to 5020; preferably the neoantigenic peptide sequences is obtained from a glioblastoma cell TE signature comprising the transcript sequences of SEQ ID NO: 381 to 393; 395 to 430 and 432 to 5020

In some embodiments, the neoantigenic peptide is encoded by an ORF sequence or a fragment thereof, from a transcript of any one of SEQ ID NO:381 to 5020. In some particular embodiments, the neoantigenic peptide is encoded by an ORF sequence or a fragment thereof, from a transcript of any one of SEQ ID NO: 381 to 430 and 432 to 5020; more particularly the neoantigenic peptide is encoded by an ORF sequence or a fragment thereof, from a transcript of any one of SEQ ID NO: 381 to 393; 395 to 430 and 432 to 5020. Typically said transcripts are translated in six frame translations (both forward and reverse direction), and the resulting amino-acid sequences are then fragmented at all stop codons to obtain TE-encoded (tumor specific neoantigenic) peptide sequences.

In some embodiments, the neoantigenic peptide comprises a sequence or a fragment thereof of any one of SEQ ID NO: 1 to 380, notably of any one of SEQ ID NO:1, 2, 9, 11, 13, 18, 22, 23, 27, 30 to 32, 35, 36, 38 to 40, 42, 45, 48 to 50, 54, 57, 58, 60, 61, 63-66, 68, 70 to 73, 76, 78, 79, 82, 83, 88, 89, 91, 93 to 95, 98, 104 to 107, 110, 111, 114, 115, 117 to 124, 126, 127, 131, 133, 138, 139, 1141, 143, 144, 150 to 153, 157, 159, 161, 162, 164, 165, 167, 172, 173, 177, 179 to 182, 188, 190, 193, 198, 199, 206, 208, 212, 214, 215, 217, 218, 222, 223, 228, 238, 239, 243 to 246, 248, 251, 253, 256, 257, 259, 260, 262, 265,267, 275, 277, 279, 281 to 283, 285 to 288, 290 to 292, 294 to 302, 304, 305, 307, 311 to 315, 317, 318, 320, 323, 325, 326, 328, 329, 331, 333 to 335, 337, 343, 344, 346, 350, 352 to 356, 359 to 362, 365, 367, 369 or 370 (non-redundant; see Table 3).

In some particular embodiments, the neoantigenic peptide comprises a sequence or a fragment thereof of any one of SEQ ID NO: 1 to 26 and 28 to 380; preferably the neoantigenic peptide comprises a sequence or a fragment thereof of any one of SEQ ID NO: 1 to 10; 12 to 26; 28 to 57; 59 to 242; 244 to 255; 257 to 319 and 321 to 380, notably of any one of SEQ ID NO:1, 2, 9, 11, 13, 18, 22, 23, 30 to 32, 35, 36, 38 to 40, 42, 45, 48 to 50, 54, 57, 60, 61, 63-66, 68, 70 to 73, 76, 78, 79, 82, 83, 88, 89, 91, 93 to 95, 98, 104 to 107, 110, 111, 114, 115, 117 to 124, 126, 127, 131, 133, 138, 139, 1141, 143, 144, 150 to 153, 157, 159, 161, 162, 164, 165, 167, 172, 173, 177, 179 to 182, 188, 190, 193, 198, 199, 206, 208, 212, 214, 215, 217, 218, 222, 223, 228, 238, 239, 244 to 246, 248, 251, 253, 257, 259, 260, 262, 265,267, 275, 277, 279, 281 to 283, 285 to 288, 290 to 292, 294 to 302, 304, 305, 307, 311 to 315, 317, 318, 323, 325, 326, 328, 329, 331, 333 to 335, 337, 343, 344, 346, 350, 352 to 356, 359 to 362, 365, 367, 369 or 370 (non-redundant; see Table 3).

In some embodiments the isolated tumor specific neoantigenic peptide comprises at least 8 amino acids, in particular 8 or 9 amino acids and binds at least one MHC class I molecule of a subject as previously defined or comprises from 13 to 25 amino acids and binds at least one MHC class II molecule of a subject as previously defined.

In some embodiments, a tumor neoantigenic peptide as per the present disclosure binds to an MHC molecule present in at least 1%, 5%, 10%, 15%, 20%, 25% or more of subjects. Notably, a tumor neoantigenic peptide as herein disclosed is expressed in at least 1%, 5%, 10%, 15%, 20%, 25% of subjects from a population of subjects suffering from a given type or tumor, for example in a population of subjects suffering from a glioblastoma.

More particularly, a tumor neoantigenic peptide of the present disclosure can elicit an immune response against a tumor present in at least 5%, 6%, 7%, 8%, 9%, 10%, 15%, 20%, 25%, 30%, 40%, 50%, 60%, 70%, 80%, 90%, 95%, or even 99% of a population of subjects suffering from a cancer, or a tumor, and more specifically from a population of subjects suffering from given type of tumor, such as glioblastoma.

In some embodiments, the isolated tumor neoantigenic peptide comprises at least 8, 9, 10, 11, or 12 amino acids, encoded by a portion of an open reading frame (ORF) from the TE transcripts of SEQ ID NO: 381 to 5020, or comprises a sequence of a fragment thereof of any one of SEQ ID NO:1 to 380. In some particular embodiments, the isolated tumor neoantigenic peptide comprises at least 8, 9, 10, 11, or 12 amino acids, encoded by a portion of an open reading frame (ORF) from the TE transcripts of SEQ ID NO: 381 to 430 and 432 to 5020, preferably SEQ ID NO: 381 to 393; 395 to 430 and 432 to 5020; or comprises a sequence of a fragment thereof of any one of SEQ ID NO: I to 26 and 28 to 380, preferably SEQ ID NO: 1 to 10; 12 to 26; 28 to 57; 59 to 242; 244 to 255; 257 to 319 and 321 to 380. The peptide may notably be 8-9, 8-10, 8-11, 12-25, 13-25, 12-20, or 13-20 amino acids in length. The N-terminus of the peptides of at least 8 amino acids may thus typically be encoded by the triplet codon starting at any of nucleotide positions 1, 4, 7, 10, 13, 16, 19 (both forward and reverse direction).

Typically, a tumor specific neoantigenic peptide as per the present disclosure may exhibit one or more of the following properties:
It does not induce an autoimmune response and/or invoke immunological tolerance when administered to a subject. Tolerating mechanisms involve clonal deletion, ignorance, anergy, or suppression in the host of the reduction in the number of high-affinity self-reactive T cells.
It is specifically expressed in tumor cells, in some embodiments it is only expressed in one or more tumor cells and not in healthy cells (e.g., not detectably expressed). Lack of expression of a neoantigenic peptide in healthy cells may for example be tested using notably the Basic local alignment search tool (BLAST) and performing alignment of the sequence of the neoantigenic peptide against the transcriptome of healthy cells.

In some embodiments, the peptide is encoded by a single genomic TE (i.e.: the peptide is non-redundant).

In other embodiments the peptide is encoded by more than one TE (i.e: the peptide is redundant). In more particular embodiments, the peptide is either highly recurrent (typically it is encoded by more than 200 genomic TE occurrences) and is non tumor specific while in other particular embodiments, the peptide has a low redundancy (typically it is encoded by less than 100 genomic TE occurrences, notably less than 50 or less than 10) and is encoded by a TE which expression is highly up-regulated in a tumor cell and/or which is not expressed in normal cells or samples (e.g., which is only expressed in at least one tumor cells, notably a glioblastoma cell).

Typically, immunization with a tumor neoantigenic peptide as per the present disclosure elicits a T cell response (i.e., is immunogenic). Assessment of the immunogenicity of a neoantigenic peptide can be achieved using an in vitro vaccination assay as described for example in the Example Section. Assessment of specific CD8⁺ T cells can be achieved by flow cytometry (Flow Cytometry and Fluorescence-Activated Cell Sorting, FACS) using multimer staining.

The neoantigenic peptide can also be modified by extending or decreasing the compound's amino acid sequence, e.g., by the addition or deletion of amino acids. The peptides can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions are generally made in accordance with the following Table 1 when it is desired to finely modulate the characteristics of the peptide.

TABLE 1

| Original residue | Exemplary substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr, Phe |
| Tyr | Trp, Phe |
| Val | Ile, Leu |
| Pro | Gly |

Substantial changes in function (e.g., affinity for MHC molecules or T cell receptors) are made by selecting substitutions that are less conservative than those in above Table 1, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lysl, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

The peptides and polypeptides may also comprise isosteres of two or more residues in the neoantigenic peptide or polypeptides. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In addition, the neoantigenic peptide may be conjugated to a carrier protein, a ligand, or an antibody. Half-life of the peptide may be improved by PEGylation, glycosylation, polysialylation, HESylation, recombinant PEG mimetics, Fc fusion, albumin fusion, nanoparticle attachment, nanoparticulate encapsulation, cholesterol fusion, iron fusion, or acylation.

Modifications of peptides and polypeptides with various amino acid mimetics or unnatural amino acids are particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides of the present disclosure is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides and polypeptides may be modified to provide desired attributes other than improved serum half-life. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the peptide may be linked to the T helper peptide without a spacer.

The neoantigenic peptide may be linked to the T helper peptide either directly or via a spacer either at the amino or carboxy terminus of the peptide. The amino terminus of either the neoantigenic peptide or the T helper peptide may be acylated. Exemplary T helper peptides include tetanus toxoid 830-843, influenza 307-319, malaria circumsporozoite 382-398 and 378-389.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Infornation's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

In a further aspect, the present disclosure provides a nucleic acid (e.g.: polynucleotide) encoding a neoantigenic peptide as herein disclosed. The polynucleotide may be selected from DNA, cDNA, PNA, CNA, RNA, either single- and/or double-stranded, or native or stabilized forms of polynucleotides, such as for example polynucleotides with a phosphorothiate backbone, or combinations thereof and it may or may not contain introns so long as it codes for the peptide. Only peptides that contain naturally occurring amino acid residues joined by naturally occurring peptide bonds are encodable by a polynucleotide. In some embodiments, the polynucleotide may be linked to a heterologous regulatory control sequence (e.g., heterologous transcriptional and/or translational regulatory control nucleotide sequences as well-known in the field).

A still further aspect of the disclosure provides an expression vector capable of expressing a neoantigenic peptide as herein disclosed. Expression vectors for different cell types are well known in the art and can be selected without undue experimentation. Generally, the DNA is inserted into an expression vector, such as a plasmid, in proper orientation and correct reading frame for expression. The expression vector will comprise the appropriate heterologous transcriptional and/or translational regulatory control nucleotide sequences recognized by the desired host. The polynucleotide encoding the tumor neoantigenic peptide may be linked to such heterologous regulatory control nucleotide sequences or may be non-adjacent yet operably linked to such heterologous regulatory control nucleotide sequences. The vector is then introduced into the host through standard techniques. Guidance can be found for example in Sambrook et al. (1989) Molecular Cloning, A Laboratory Manual, Cold Spring Harbor Laboratory, Cold Spring Harbor, N.Y.

### Antigen Presenting Cells (APCs)

The present disclosure also encompasses a population of antigen presenting cells that have been pulsed with one or more of the peptides as previously defined and/or obtainable in a method as previously described. Preferably, the antigen presenting cells are dendritic cell (DCs) or artificial antigen presenting cells (aAPCs) (see Neal, Lillian R et al. “The Basics of Artificial Antigen Presenting Cells in T Cell-Based Cancer Immunotherapies.” Journal of immunology research and therapy vol. 2, 1 (2017): 68-79). Dendritic cells (DC) are professional antigen-presenting cells (APC) that have an extraordinary capacity to stimulate naive T-cells and initiate primary immune responses to pathogens. Indeed, the main role of mature DCs are to sense antigens and produce mediators that activate other immune cells, particularly T cells. DCs are potent stimulators for lymphocyte activation as they express MHC molecules that trigger TCRs (signal 1) and co-stimulatory molecules (signal 2) on T cells. Additionally, DCs also secrete cytokines that support T cell expansion. T cells require presented antigen in the form of a processed peptide to recognize foreign pathogens or tumor. Presentation of peptide epitopes derived from pathogen/tumor proteins is achieved through MHC molecules. MHC class I (MHC-I) and MHC class II (MHC-II) molecules present processed peptides to CD8+ T cells and CD4+ T cells, respectively. Importantly, DCs home to inflammatory sites containing abundant T cell populations to foster an immune response. Thus, DCs can be a crucial component of any immunotherapeutic approach, as they are intimately involved with the activation of the adaptive immune response. In the context of vaccines, DC therapy can enhance T cell immune responses to a desired target in healthy volunteers or patients with infectious disease or cancer. In one embodiment, APCs are artificial APC, which are genetically modified to express the desired T-cell co-stimulatory molecules, human HLA alleles and/or cytokines. Such artificial antigen presenting cells (aAPC) can provide the requirements for adequate T-cell engagement, co-stimulation, as well as sustained release of cytokines that allow for controlled T-cell expansion. These cells are not subject to the constraints of time and limited availability and can be stored in small aliquots for subsequent use in generating T-cell lines from different donors, thus representing an off the shelf reagent for immunotherapy applications. Expression of potent co-stimulatory signals on these aAPC endows this system with higher efficiency lending to increased efficacy of adoptive immunotherapy. Furthermore, aAPC can be engineered to express genes directing release of specific cytokines to facilitate the preferential expansion of desirable T-cell subsets for adoptive transfer; such as long lived memory T-cells (see for review Hasan A H et al., Artificial Antigen Presenting Cells: An Off the Shelf Approach for Generation of Desirable T-Cell Populations for Broad Application of Adoptive Immunotherapy; Adv Genet Eng. 2015; 4(3): 130, Kim J V, Latouche J B, Rivière I, Sadelain M. The ABCs of artificial antigen presentation. Nat Biotechnol. 2004; 22:403-410 or Wang C, Sun W, Ye Y, Bomba H N, Gu Z. Bioengineering of Artificial Antigen Presenting Cells and Lymphoid Organs. Theranostics 2017; 7(14):3504-3516.).

Typically, the dendritic cells are autologous dendritic cells that are pulsed with a neoantigenic peptide as herein disclosed. The peptide may be any suitable peptide that gives rise to an appropriate T-cell response. The antigen-presenting cell (or stimulator cell) typically has an MHC class I or II molecule on its surface, and in one embodiment is substantially incapable of itself loading the MHC class I or II molecule with the selected antigen. The MHC class I or II molecule may readily be loaded with the selected antigen in vitro.

As an alternative the antigen presenting cell may comprise an expression construct encoding a tumor neoantigenic peptide as herein disclosed. The polynucleotide may be any suitable polynucleotide as previously defined and it is preferred that it is capable of transducing the dendritic cell, thus resulting in the presentation of a peptide and induction of immunity.

Thus, the present disclosure encompasses a population of APCs than can be pulsed or loaded with the neoantigenic peptide as herein disclosed, genetically modified (via DNA or RNA transfer) to express at least one neoantigenic peptide as herein disclosed, or that comprise an expression construct encoding a tumor neoantigenic peptide of the present disclosure as well as a method of producing thereof. Typically, the population of APCs is pulsed or loaded, modified to express or comprises at least one, at least 5, at least 10, at least 15, or at least 20 different neoantigenic peptide or expression construct encoding it.

The present disclosure also encompasses compositions comprising APCs as herein disclosed. APCs can be suspended in any known physiologically compatible pharmaceutical carrier, such as cell culture medium, physiological saline, phosphate-buffered saline, cell culture medium, or the like, to form a physiologically acceptable, aqueous pharmaceutical composition. Parenteral vehicles include sodium chloride solution, Ringer's dextrose, dextrose and sodium chloride, lactated Ringer's. Other substances may be added as desired such as antimicrobials. As used herein, a “carrier” refers to any substance suitable as a vehicle for delivering an APC to a suitable in vitro or in vivo site of action. As such, carriers can act as an excipient for formulation of a therapeutic or experimental reagent containing an APC. Preferred carriers are capable of maintaining an APC in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

### Vaccine Compositions

The present disclosure further encompasses a vaccine or immunogenic composition capable of raising a specific T-cell response comprising:
one or more neoantigenic peptides as herein defined,
one or more polynucleotides encoding a neoantigenic peptide as herein defined; and/or
a population of antigen presenting cells (such as autologous dendritic cells or artificial APC) as described above.

A suitable vaccine or immunogenic composition will preferably contain between 1 and 20 neoantigenic peptides, more preferably 2, 3, 4, 5, 6, 7, 8, 9, 10, 11, 12, 13, 14, 15, 16, 17, 18, 19, 20, 21, 22, 23, 24, 25 different neoantigenic peptides, further preferred 6, 7, 8, 9, 10 11, 12, 13, or 14 different neoantigenic peptides, and most preferably 12, 13 or 14 different neoantigenic peptides.

The neoantigenic peptide(s) may be linked to a carrier protein. Where the composition contains two or more neoantigenic peptides, the two or more (e.g.: 2-25) peptides may be linearly linked by a spacer molecule as described above, e.g., a spacer comprising 2-6 nonpolar or neutral amino acids.

In one embodiment of the present disclosure the different neoantigenic peptides, encoding polynucleotides, vectors, or APCs are selected so that one vaccine or immunogenic composition comprises neoantigenic peptides capable of associating with different MHC molecules, such as different MHC class I molecules. Preferably, such neoantigenic peptides are capable of associating with the most frequently occurring MHC class I molecules, e.g., different fragments capable of associating with at least 2 preferred, more preferably at least 3 preferred, even more preferably at least 4 preferred MHC class I molecules. In some embodiments, the compositions comprise peptides, encoding polynucleotides, vectors, or APCs capable of associating with one or more MHC class II molecules. The MHC is optionally HLA-A, —B, —C, -DP, -DQ, or -DR.

The vaccine or immunogenic composition is capable of raising a specific cytotoxic T-cells response and/or a specific helper T-cell response.

Thus, in a particular embodiment, the present disclosure also relates to a neoantigenic peptide as described above, wherein the neoantigenic peptide has a tumor specific neoepitope and is included in a vaccine or immunogenic composition. A vaccine composition is to be understood as meaning a composition for generating immunity for the prophylaxis and/or treatment of diseases. Accordingly, vaccines are medicines which comprise or generate antigens and are intended to be used in humans or animals for generating specific defense and protective substance by vaccination. An “immunogenic composition” is to be understood as meaning a composition that comprises or generates antigen(s) and is capable of eliciting an antigen-specific humoral or cellular immune response, e.g.: T-cell response.

In a preferred embodiment, the neoantigenic peptide according to the disclosure is 8 or 9 residues long, or from 13 to 25 residues long. When the peptide is less than 20 residues, to have a peptide better suited for in vivo immunization, said neoantigenic peptide, is optionally flanked by additional amino acids to obtain an immunization peptide of more amino acids, usually more than 20.

Pharmaceutical compositions (i.e., the vaccine or immunogenic composition) comprising a peptide as herein described may be administered to an individual already suffering from a cancer or a tumor. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific CTL activity in the patient's blood. It must be kept in mind that the peptide and compositions of the present invention may generally be employed in serious disease states, that is, life-threatening or potentially life-threatening situations, especially when the cancer has metastasized. In such cases, in view of the minimization of extraneous substances and the relative nontoxic nature of the peptide, it is possible and may be felt desirable by the treating physician to administer substantial excesses of these peptide compositions.

For therapeutic use, administration should begin at the detection or surgical removal of tumors. This is followed by boosting doses until at least symptoms are substantially abated and for a period thereafter.

The vaccine or immunogenic compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

The vaccine or immunogenic composition may be a pharmaceutical composition which additionally comprises a pharmaceutically acceptable adjuvant, immunostimulatory agent, stabilizer, carrier, diluent, excipient and/or any other materials well known to those skilled in the art. Such materials should be non-toxic and should not interfere with the efficacy of the active ingredient. The carrier is preferably an aqueous carrier, but its precise nature of the carrier or other material will depend on the route of administration. A variety of aqueous carriers may be used, e.g., water, buffered water, 0.9% saline, 0.3% glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may further contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. See, for example, Butterfield, B M J. 2015 22; 350 for a discussion of cancer vaccines.

Example adjuvants that increase or expand the immune response of a host to an antigenic compound include emulsifiers, muramyl dipeptides, avridine, aqueous adjuvants such as aluminum hydroxide, chitosan-based adjuvants, saponins, oils, Amphigen, LPS, bacterial cell wall extracts, bacterial DNA, CpG sequences, synthetic oligonucleotides, cytokines and combinations thereof. Emulsifiers include, for example, potassium, sodium and ammonium salts of lauric and oleic acid, calcium, magnesium and aluminum salts of fatty acids, organic sulfonates such as sodium lauryl sulfate, cetyltrhethylammonlum bromide, glycerylesters, polyoxyethylene glycol esters and ethers, and sorbitan fatty acid esters and their polyoxyethylene, acacia, gelatin, lecithin and/or cholesterol. Adjuvants that comprise an oil component include mineral oil, a vegetable oil, or an animal oil. Other adjuvants include Freund's Complete Adjuvant (FCA) or Freund's Incomplete Adjuvant (FIA). Cytokines useful as additional immunostimulatory agents include interferon alpha, interleukin-2 (IL-2), and granulocyte macrophage-colony stimulating factor (GM-CSF), or combinations thereof.

The concentration of peptides as herein described in the vaccine or immunogenic formulations can vary widely, i.e., from less than about 0.10%, usually at or at least about 2% to as much as 20% to 50% or more by weight, and will be selected primarily by fluid volumes, viscosities, etc., in accordance with the mode of administration selected.

The peptides as herein described may also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871; 4,501,728; 4,837,028; and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10⁻⁹⁵% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and antigen presenting cell (APC) is present. Correspondingly, it may enhance the immune response if not only the peptide is used for activation of CTLs, but if additionally, APCs with the respective MHC molecule are added. Therefore, in some embodiments the vaccine or immunogenic composition according to the present disclosure alternatively or additionally contains at least one antigen presenting cell, preferably a population of APCs.

The vaccine or immunogenic composition may thus be delivered in the form of a cell, such as an antigen presenting cell, for example as a dendritic cell vaccine. The antigen presenting cells such as a dendritic cell may be pulsed or loaded with a neoantigenic peptide as herein disclosed, may comprise an expression construct encoding a neoantigenic peptide as herein disclosed, or may be genetically modified (via DNA or RNA transfer) to express one, two or more of the herein disclosed neoantigenic peptides, for example at least 2, 3, 4, 5, 6, 7, 8, 9, or 10 neoantigenic peptides.

Suitable vaccines or immunogenic compositions may also be in the form of DNA or RNA relating to neoantigenic peptides as described herein. For example, DNA or RNA encoding one or more neoantigenic peptides or proteins derived therefrom may be used as the vaccine, for example by direct injection to a subject. For example, DNA or RNA encoding at least 2, 3, 4, 5, 6, 7, 8, 9, 10, 11, 12, 13, 14, 15, 16, 17, 18, 19, 20, 21, 22, 23, 24 or 25 neoantigenic peptides or proteins derived therefrom.

Several methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833; WO 91/06309; and Felgner et al., Proc. Natl. Acad. Sci. USA 84: 7413-7414 (1987).

Delivery systems may optionally include cell-penetrating peptides, nanoparticulate encapsulation, virus like particles, liposomes, or any combination thereof. Cell penetrating 20 peptides include TAT peptide, herpes simplex virus VP22, transportan, Antp. Liposomes may be used as a delivery system. Listeria vaccines or electroporation may also be used.

The one or more neoantigenic peptides may also be delivered via a bacterial or viral vector containing DNA or RNA sequences which encode one or more neoantigenic peptides. The DNA or RNA may be delivered as a vector itself or within attenuated bacteria virus or live attenuated virus, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhivectors and the like, will be apparent to those skilled in the art from the description herein.

An appropriate mean of administering nucleic acids encoding the peptides as herein described involves the use of minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include helper T lymphocyte, epitopes, a leader (signal) sequence, and an endoplasmic reticulum retention signal. In addition, MHC presentation of CTL epitopes may be improved by including synthetic (e.g.: poly-alanine) or naturally occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified, and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Thus, the DNA or RNA encoding the neoantigenic peptide(s) may typically be operably linked to one or more of:
a promoter that can be used to drive nucleic acid molecule expression. AAV ITR can serve as a promoter and is advantageous for eliminating the need for an additional promoter element. For ubiquitous expression, the following promoters can be used: CMV (notably human cytomegalovirus immediate early promoter (hCMV-IE)), CAG, CBh, PGK, SV40, RSV, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA). Typically, the promoter includes a down-stream cloning site for minigene insertion. For examples of suitable promoter sequences, see notably U.S. Pat. Nos. 5,580,859 and 5,589,466.
Transcriptional transactivators or other enhancer elements, which can also increase transcription activity, e.g.: the regulatory R region from the 5′ long terminal repeat (LTR) of human T-cell leukemia virus type 1 (HTLV-1) (which when combined with a CMV promoter has been shown to induce higher cellular immune response).
Translation optimizing sequences e.g.: a Kozak sequence flanking the AUG initiator codon (ACCAUGG) within mRNA, and codon optimization.

Additional vector modifications may be desired to optimize minigene expression and immunogenicity. In some cases, introns are required for efficient gene expression, and one or more synthetic or naturally occurring introns could be incorporated into the transcribed region of the minigene. The inclusion of mRNA stabilization sequences can also be considered for increasing minigene expression. It has recently been proposed that immunostimulatory sequences (ISSs or CpGs) play a role in the immunogenicity of DNA' vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used.

DNA vaccines or immunogenic compositions as herein described can be enhanced by co-delivering cytokines that promote cell-mediated immune responses, such as IL-2, IL-12, IL-18, GM-CSF and IFNγ. CXC chemokines such as IL-8, and CC chemokines such as macrophage inflammatory protein (MIP)-1α, MIP-3α, MIP-3β, and RANTES, may increase the potency of the immune response. DNA vaccine immunogenicity can also be enhanced by co-delivering plasmid-encoded cytokine-inducing molecules (e.g.: LeIF), co-stimulatory and adhesion molecules, e.g.: B7-1 (CD80) and/or B7-2 (CD86). Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g.: TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Vaccines or immunogenic compositions comprising peptides may be administered in combination with vaccines or immunogenic compositions comprising polynucleotide encoding the peptides. For example, administration of peptide vaccine and DNA vaccine may be alternated in a prime-boost protocol. For example, priming with a peptide immunogenic composition and boosting with a DNA immunogenic composition is contemplated, as is priming with a DNA immunogenic composition, and boosting with a peptide immunogenic composition.

The present disclosure also encompasses a method for producing a vaccine composition comprising the steps of:
a) optionally, identifying at least one neoantigenic peptide according to the method as previously described;
b) producing said at least one neoantigenic peptide, at least one polypeptide encoding neoantigenic peptide(s), or at least a vector comprising said polypeptide(s) as described herein; and
c) optionally adding physiologically acceptable buffer, excipient and/or adjuvant and producing a vaccine with said at least one neoantigenic peptide, polypeptide, or vector.

Another aspect of the present disclosure is a method for producing a DC vaccine, wherein said DCs present at least one neoantigenic peptide as herein disclosed or expresses at least one expression construct encoding a tumor neoantigenic peptide as herein disclosed.

### Antibodies TCRs, CARs and Derivatives Thereof

The present disclosure also relates to an antibody or an antigen-binding fragment thereof that specifically binds a neoantigenic peptide as herein defined.

In some embodiments, the neoantigenic peptide is in association with an MHC or HLA molecule.

Typically, said antibody, or antigen-binding fragment thereof binds a neoantigenic peptide as herein defined, alone or optionally in association with an MHC or HLA molecule, with a K_{d} binding affinity of 10⁻⁷ M or less, 10⁻⁸ M or less, 10⁻⁹ M or less, 10⁻¹⁰ M or less, or 10⁻¹¹ M or less.

To promote the infiltration and recognition of tumor cells by lymphocytes T (LT), another strategy consists in using antibodies capable of recognizing more than one antigenic target simultaneously and more particularly two antigenic targets simultaneously. There are many formats of bispecific antibodies. BiTE (bi-specific T-cell engager) are the first to have been developed. These are proteins of fusion consisting of two scFvs (variable domains heavy VH and light VL chains) from two antibodies linked by a binding peptide: one recognizes the LT marker (CD3⁺) and the other a tumor antigen. The goal is to favor recruitment and activation of LTs in contact with tumor, thus leading to cell lysis tumor (See for review: Patrick A. Baeuerle and Carsten Reinhardt; Bispecific T-Cell Engaging Antibodies for Cancer Therapy; Cancer Res 2009; 69: (12). Jun. 15, 2009; and Galaine et al., Innovations & Thérapeutiques en Oncologie, vol. 3-n° 3-7, mai-août 2017).

In a particular embodiment, said antibody is a bi-specific T-cell engager that targets a tumor neoantigenic peptide as herein defined, optionally in association with an MHC or an HLA molecule and which further targets at least an immune cell antigen. Typically, the immune cell is a T cell, a NK cell, or a dendritic cell. In this context, the targeted immune cell antigen may be for example CD3, CD16, CD30 or a TCR.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., VHH antibodies, sdAb, sdFv, nanobody) fragments. The term encompasses genetically engineered and/or otherwise variants modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody and fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments, the antibody comprises a light chain variable domain and a heavy chain variable domain, e.g.: in an scFv format.

Antibodies include variant polypeptide species that have one or more amino acid substitutions, insertions, or deletions in the native amino acid sequence, provided that the antibody retains or substantially retains its specific binding function. Conservative substitutions of amino acids are well known and described above.

The present disclosure further includes a method of producing an antibody, or antigen-binding fragment thereof, comprising a step of selecting antibodies that bind to a tumor neoantigen peptide as herein defined, optionally in association with an MHC or HLA molecule, with a K_{d}binding affinity of about 106 M or less, 10⁻⁷ M or less, 10⁻⁸ M or less, 10⁻⁹ M or less, 1010 M or less, or 10⁻¹¹ M or less.

In some embodiments, the antibodies are selected from a library of human antibody sequences. In some embodiments, the antibodies are generated by immunizing an animal with a polypeptide comprising the neoantigenic peptide, optionally in association with an MHC or HLA molecule, followed by the selection step.

Antibodies including chimeric, humanized, or human antibodies can be further affinity matured and selected as described above. Humanized antibodies contain rodent-sequence derived CDR regions; typically, the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence and are typically produced via phage display technologies of human antibody libraries, or immunization of transgenic mice whose native immunoglobin loci have been replaced with segments of human immunoglobulin loci.

Antibodies produced by said method, as well as immune cells expressing such antibodies or fragments thereof are also encompassed by the present disclosure.

The present disclosure also encompasses pharmaceutical compositions comprising one or more antibodies as herein disclosed alone or in combination with at least one other agent, such as a stabilizing compound, which may be administered in any sterile, biocompatible pharmaceutical carrier and optionally formulated with formulated with sterile pharmaceutically acceptable buffer(s), diluent(s), and/or excipient(s). Pharmaceutically acceptable carriers typically enhance or stabilize the composition, and/or can be used to facilitate preparation of the composition. Pharmaceutically acceptable carriers include solvents, dispersion media, coatings, antibacterial and antifungal agents, isotonic and absorption delaying agents, and the like that are physiologically compatible and, in some embodiments, pharmaceutically inert.

Administration of pharmaceutical composition comprising antibodies as herein disclosed can be accomplished orally or parenterally. Methods of parenteral delivery include topical, intra-arterial (directly to the tumor), intramuscular, spinal, subcutaneous, intramedullary, intrathecal, intraventricular, intravenous, intraperitoneal, or intranasal administration.

Thus, in addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Depending on the route of administration, the active compound, i.e., antibody, bispecific and multispecific molecule, may be coated in a material to protect the compound from the action of acids and other natural conditions that may inactivate the compound.

The composition is typically sterile and preferably fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical compositions of the disclosure can be prepared in accordance with methods well known and routinely practiced in the art. See. e.g., Remington: The Science and Practice of Pharmacy, Mack Publishing Co., 20th ed., 2000; and Sustained and Controlled Release Drug Delivery Systems, J R. Robinson, ed., Marcel Dekker, Inc., New York, 1978. Pharmaceutical compositions are preferably manufactured under GMP conditions.

The present disclosure also encompasses a T cell receptor (TCR) that targets a neoantigenic peptide as herein defined in association with an MHC or HLA molecule.

The present disclosure further includes a method of producing a TCR, or an antigen-binding fragment thereof, comprising a step of selecting TCRs that bind to a tumor neoantigen peptide as herein defined, optionally in association with an MHC or HLA molecule, optionally with a K_{d} binding affinity of about 10⁻⁶ M or less, 10⁻⁷ M or less, 10⁻⁸ M or less, 10⁻⁹ M or less, 10⁻¹⁰ M or less, or 10⁻¹¹ M or less.

Nucleic acid encoding the TCR can be obtained from a variety of sources, such as by polymerase chain reaction (PCR) amplification of naturally occurring TCR DNA sequences, followed by expression of antibody variable regions, followed by the selecting step described above. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and L1 (2005) Nat Biotechnol. 23:349-354.

A “T cell receptor” or “TCR” refers to a molecule that contains a variable α and β chains (also known as TCRa and TCRp, respectively) or a variable γ and δ chains (also known as TCRγ and TCRδ, respectively) and that is capable of specifically binding to an antigen peptide bound to a MHC receptor. In some embodiments, the TCR is in the UP form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3^{rd} Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the up form or γδ form.

Thus, for purposes herein, reference to a TCR includes any TCR or functional fragment, such as an antigen-binding portion of a TCR that binds to a specific antigenic peptide bound in an MHC molecule, i.e., MHC-peptide complex. An “antigen-binding portion” or antigen-binding fragment” of a TCR, which can be used interchangeably, refers to a molecule that contains a portion of the structural domains of a TCR, but that binds the antigen (e.g.: MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Pwc. Nat'lAcad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3ζ chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3− and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

While T-cell receptors (TCRs) are transmembrane proteins and do not naturally exist in soluble form, antibodies can be secreted as well as membrane bound. Importantly, TCRs have the advantage over antibodies that they in principle can recognize peptides generated from all degraded cellular proteins, both intra- and extracellular, when presented in the context of MHC molecules. Thus, TCRs have important therapeutic potential.

The present disclosure also relates to soluble T-cell receptors (sTCRs) that contain the antigen recognition part directed against a tumor neoantigenic peptide as herein disclosed (see notably Walseng E, Wilchli S, Fallang L-E, Yang W, Vefferstad A, Areffard A, et al. (2015) Soluble T-Cell Receptors Produced in Human Cells for Targeted Delivery. PLoS ONE 10(4): e0119559). In a particular embodiment, the soluble TCR can be fused to an antibody fragment directed to a T cell antigen, optionally wherein the targeted antigen is CD3 or CD16 (see for example Boudousquie, Caroline et al. “Polyfunctional response by ImmTAC (IMCgp100) redirected CD8+ and CD4+ T cells.” Immunology vol. 152, 3 (2017): 425-438. doi:10.1111/imm.12779).

The present disclosure also encompasses a chimeric antigen receptor (CAR) which is directed against a tumor neoantigenic peptide as herein disclosed. CARs are fusion proteins comprising an antigen-binding domain, typically derived from an antibody, linked to the signalling domain of the TCR complex. CARs can be used to direct immune cells such T-cells or NK cells against a tumor neoantigenic peptide as previously defined with a suitable antigen-binding domain selected.

The antigen-binding domain of a CAR is typically based on a scFv (single chain variable fragment) derived from an antibody. In addition to an N-terminal, extracellular antibody-binding domain, CARs typically may comprise a hinge domain, which functions as a spacer to extend the antigen-binding domain away from the plasma membrane of the immune effector cell on which it is expressed, a transmembrane (TM) domain, an intracellular signalling domain (e.g.: the signalling domain from the zeta chain of the CD3 molecule (CD3ζ) of the TCR complex, or an equivalent) and optionally one or more co-stimulatory domains which may assist in signalling or functionality of the cell expressing the CAR. Signalling domains from co-stimulatory molecules including CD28, OX-40 (CD134), ICOS-1, CD27, GITR, CD28, DAP10, and 4-1BB (CD137) can be added alone (second generation) or in combination (third generation) to enhance survival and increase proliferation of CAR modified T cells.

Thus, the CAR may include:
(1) In its extracellular portion, one or more antigen binding molecules, such as one or more antigen-binding fragment, domain, or portion of an antibody, or one or more antibody variable domains (heavy chain and/or light chain), and/or antibody molecules.
(2) In its transmembrane portion, a transmembrane domain derived from human T cell receptor-alpha or -beta chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR. In some embodiments, the transmembrane domain is derived from CD28, CD8 or CD3-zeta.
(3) One or more co-stimulatory domains, such as co-stimulatory domains derived from human CD28, 4-1BB (CD137), ICOS-1, CD27, OX 40 (CD137), DAP10, and GITR (AITR). In some embodiments, the CAR comprises co-stimulating domains of both CD28 and 4-1BB.
(4) In its intracellular signalling domain, one or more intracellular signalling domain(s) comprising one or more ITAMs, for example: the intracellular signalling domain or a portion thereof from CD3-zeta, or a variant thereof lacking one or two ITAMs (e.g.: ITAM3 and/or ITAM2), FcR gamma, FcR beta, CD3 gamma, CD3 delta, CD3 epsilon, CDS, CD22, CD79a, CD79b, and/or CD66d, notably selected from the intracellular domain of CD3-zeta, or a variant thereof lacking one or two ITAMs (e.g.: ITAM3 and ITAM2), or the intracellular signalling of FcRIy or a variant thereof.

The CAR can be designed to recognize tumor neoantigenic peptide alone or in association with an HLA or MHC molecule.

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.: WO2014/055668.

The present disclosure also encompasses polynucleotides encoding antibodies, antigen-binding fragments or derivatives thereof, TCRs and CARs as previously described as well as vector comprising said polynucleotide(s).

### Immune Cells

The present disclosure further encompasses immune cells which target one or more tumor neoantigenic peptides as previously described.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, the term “T cell” includes cells bearing a T cell receptor (TCR), in particular TCR directed against a tumor neoantigenic peptide as herein disclosed. T-cells according to the present disclosure can be selected from the group consisting of inflammatory T-lymphocytes, cytotoxic T-lymphocytes, regulatory T-lymphocytes, Mucosal-Associated Invariant T cells (MAIT), Yδ T cell, tumour infiltrating lymphocyte (TILs) or helper T-lymphocytes included both type 1 and 2 helper T cells and Thl7 helper cells. In another embodiment, said cell can be derived from the group consisting of CD4⁺ T-lymphocytes and CD8⁺ T-lymphocytes. Said immune cells may originate from a healthy donor or from a subject suffering from a cancer, or a tumor.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34⁺ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation.

In another embodiment, said cell can be derived from a healthy donor, from a subject diagnosed with cancer or tumor, notably with glioblastoma. The cell can be autologous or allogeneic.

In allogeneic immune cell therapy, immune cells are collected from healthy donors, rather than the patient. Typically these are HLA matched to reduce the likelihood of graft vs. host disease. Alternatively, universal ‘off the shelf’ products that may not require HLA matching comprise modifications designed to reduce graft vs. host disease, such as disruption or removal of the TCRαβ receptor. See Graham et al., Cells. 2018 October; 7(10): 155 for a review. Because a single gene encodes the alpha chain (TRAC) rather than the two genes encoding the beta chain, the TRAC locus is a typical target for removing or disrupting TCRa (3 receptor expression. Alternatively, inhibitors of TCRαβ signalling may be expressed, e.g. truncated forms of CD3ζ can act as a TCR inhibitory molecule. Disruption or removal of HLA class I molecules has also been employed. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression. Ren et al. simultaneously knocked out TCRαβ, B2M and the immune-checkpoint PD1. Generally, the immune cells are activated and expanded to be utilized in the adoptive cell therapy. The immune cells as herein disclosed can be expanded in vivo or ex vivo. The immune cells, in particular T-cells can be activated and expanded generally using methods known in the art. Generally, the T-cells are expanded by contact with a surface having attached thereto an agent that stimulates a CD3/TCR complex associated signal and a ligand that stimulates a co-stimulatory molecule on the surface of the T cells.

In one embodiment of the present disclosure, the immune cell can be modified to be directed to tumor neoantigenic peptides as previously defined. In a particular embodiment, said immune cell may express a recombinant antigen receptor directed to said neoantigenic peptide its cell surface. By “recombinant” is meant an antigen receptor which is not encoded by the cell in its native state, i.e., it is heterologous, non-endogenous. Expression of the recombinant antigen receptor can thus be seen to introduce new antigen specificity to the immune cell, causing the cell to recognise and bind a previously described peptide. The antigen receptor may be isolated from any useful source. In some embodiments, the cells comprise one or more nucleic acids introduced via genetic engineering that encode one or more antigen receptors, wherein the antigen include at least one tumor neoantigenic peptide as per the present disclosure.

Among the antigen receptors as per the present disclosure are genetically engineered T cell receptors (TCRs) and components thereof, as well as functional non-TCR antigen receptors, such as chimeric antigen receptors (CAR) as previously described.

Methods by which immune cells can be genetically modified to express a recombinant antigen receptor are well known in the art. A nucleic acid molecule encoding the antigen receptor may be introduced into the cell in the form of e.g.: a vector, or any other suitable nucleic acid construct. Vectors, and their required components, are well known in the art. Nucleic acid molecules encoding antigen receptors can be generated using any method known in the art, e.g.: molecular cloning using PCR. Antigen receptor sequences can be modified using commonly used methods, such as site-directed mutagenesis.

The present disclosure also relates to a method for providing a T cell population which targets a tumor neoantigenic peptide as herein disclosed.

The T cell population may comprise CD8⁺ T cells, CD4⁺ T cells or CD8⁺ and CD4⁺ T cells.

T cell populations produced in accordance with the present disclosure may be enriched with T cells that are specific to, i.e.: target, the tumor neoantigenic peptide of the present disclosure. That is, the T cell population that is produced in accordance with the present disclosure will have an increased number of T cells that target one or more tumor neoantigenic peptide. For example, the T cell population of the disclosure will have an increased number of T cells that target a tumor neoantigenic peptide compared with the T cells in the sample isolated from the subject. That is to say, the composition of the T cell population will differ from that of a “native” T cell population (i.e.: a population that has not undergone the identification and expansion steps discussed herein), in that the percentage or proportion of T cells that target a tumor neoantigenic peptide will be increased.

T cell populations produced in accordance with the present disclosure may be enriched with T cells that are specific to, i.e.: target, tumor neoantigenic peptide. That is, the T cell population that is produced in accordance with the present disclosure will have an increased number of T cells that target one or more tumor neoantigenic peptide of the present disclosure. For example, the T cell population of the present disclosure will have an increased number of T cells that target a tumor neoantigenic peptide compared with the T cells in the sample isolated from the subject. That is to say, the composition of the T cell population will differ from that of a “native” T cell population (i.e.: a population that has not undergone the identification and expansion steps discussed herein), in that the percentage or proportion of T cells that target a tumor neoantigenic peptide will be increased.

The T cell population according to the present disclosure may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a tumor neoantigenic peptide as herein disclosed. For example, the T cell population may have about 0.2%-5%, 5%-10%, 10⁻²⁰%, 20-30%, 30-40%, 40-50%, 50-70% or 70-100% T cells that target a tumor neoantigenic peptide of the present disclosure.

An expanded population of tumor neoantigenic peptide-reactive T cells may have a higher activity than a population of T cells not expanded, for example, using a tumor neoantigenic peptide. Reference to “activity” may represent the response of the T cell population to restimulation with a tumor neoantigenic peptide, e.g.: a peptide corresponding to the peptide used for expansion, or a mix of tumor neoantigenic peptide. Suitable methods for assaying the response are known in the art. For example, cytokine production may be measured (e.g.: IL2 or IFNγ production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

In some embodiments, the present disclosure provides a plurality of T cells or a population of T cells wherein said plurality, or population, of T cells comprises at least a T cell which recognizes a clonal tumor neoantigenic peptide and at least another T cell which recognizes a different clonal tumor neoantigenic peptide. As such, the present disclosure provides a plurality of T cells which recognize different clonal tumor neoantigenic peptides. Different T cells in the plurality or population may alternatively have different TCRs which recognize the same tumor neoantigenic peptide.

In some embodiments the number of clonal tumor neoantigenic peptides recognized by the plurality of T cells is from 2 to 1000. For example, the number of clonal neo-antigens recognized may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, preferably 2 to 100. There may be a plurality of T cells with different TCRs, but which recognize the same clonal neo-antigen.

The T cell population may be all or primarily composed of CD8⁺ T cells, or all or primarily composed of a mixture of CD8⁺ T cells and CD4⁺ T cells or all or primarily composed of CD4⁺ T cells.

In particular embodiments, the T cell population is generated from T cells isolated from a subject with a tumor. For example, the T cell population may be generated from T cells in a sample isolated from a subject with a tumor. The sample may be a tumor sample, a peripheral blood sample or a sample from other tissues of the subject.

In a particular embodiment the T cell population is generated from a sample from the tumor in which the tumor neoantigenic peptide is identified. In other words, the T cell population is isolated from a sample derived from the tumor of a patient to be treated. Such T cells are referred to herein as “tumor infiltrating lymphocytes” (TILs).

T cells may be isolated using methods which are well known in the art. For example, T cells may be purified from single cell suspensions generated from samples, based on expression of CD3*, CD4⁺ or CD8⁺ T cells, may be enriched from samples by passage through a Ficoll-plaque gradient.

### Cancer Therapeutic and Diagnostic Methods

In any of the embodiments, the Cancer Therapeutic Products described herein may be used in methods for inhibiting proliferation of cancer cells. The Cancer Therapeutic Products described herein may also be used in the treatment of cancer or tumor as previously listed, or for the prophylactic treatment of such cancer, in patients at risk of such cancer or tumor.

Cancers that can be treated using the therapy described herein include any solid or non-solid tumors. In a specific embodiment of the present disclosure, the tumor is glioblastoma.

Cancers includes also the cancers which are refractory to treatment with other chemotherapeutics. The term “refractory”, as used herein refers to a cancer (and/or metastases thereof), which shows no or only weak antiproliferative response (e.g., no, or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

A subject as per the present disclosure is typically a patient in need thereof that has been diagnosed with tumor. The subject is typically a mammal, notably a human, dog, cat, horse, or any animal in which a tumor specific immune response is desired.

The present disclosure also pertains to a neoantigenic peptide, a population of APCs, a vaccine or immunogenic composition, a polynucleotide encoding a neoantigenic peptide or a vector as previously defined for use in cancer vaccination therapy of a subject or for treating cancer in a subject, wherein the peptide(s) binds at least one MHC molecule of said subject.

The present disclosure also provides a method for treating cancer in a subject, comprising administering a vaccine or immunogenic composition as described herein to said subject in a therapeutically effective amount to treat the subject. The method may additionally comprise the step of identifying a subject who has a cancer or a tumor, notably a glioblastoma.

The present disclosure also relates to a method of treating cancer, typically a glioblastoma, comprising producing an antibody or antigen-binding fragment thereof by the method as herein described and administering to a subject with cancer, or tumor said antibody or antigen-binding fragment thereof, or with an immune cell expressing said antibody or antigen-binding fragment thereof, in a therapeutically effective amount to treat said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MHC or HLA molecule, for use in cancer therapy of a subject, notably glioblastoma therapy, wherein the tumor neoantigenic peptide binds at least one MHC molecule of said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MHC or HLA molecule, or an immune cell which targets a neoantigenic peptide, as previously defined, for use in adoptive cell or CAR-T cell therapy in a subject, wherein the tumor neoantigenic peptide binds at least one MHC molecule of said subject.

Typically, the skilled person is able to select an appropriate antigen receptor which binds and recognizes a tumor neoantigenic peptide as previously defined with which to redirect an immune cell to be used for use in cancer cell therapy, notably glioblastoma cell therapy. In a particular embodiment, the immune cell for use in the method of the present disclosure is a redirected T-cell, e.g., a redirected CD8⁺ and/or CD4⁺ T-cell.

The inventors herein provide a method for identifying or screening population specific TE-signature, and in particular tumor cell specific TE-signature. This discovery has strong potentials in diagnostic. Indeed, it provides tumor-specific biomarkers that are shared among patients and that can differentiate neoplastic cells from other cell populations from the core tumor and/or the tumor microenvironment but also neoplastic cells from different type of tumors.

The present disclosure therefore also encompasses a method for the diagnostic of a tumor, such as for example a glioblastoma. Said method comprises the identification, as per the method as herein disclosed, in a tumor sample obtained from a patient a tumor cell specific TE signature as herein defined.

The present application also encompasses a method for treating a patient suffering from a tumor, notably suffering from a tumor associated with de-repressed TEs, notably suffering from glioblastoma tumor comprising a step of diagnosing said tumor as per the method as above defined and a step of administering a treatment dedicated to the identified tumor.

In some embodiment, the present application relates to a method for treating a patient suffering from a tumor, notably suffering from a tumor associated with de-repressed TEs, notably suffering from a glioblastoma tumor, comprising (i) a step of diagnosing said tumor as per the method as above defined and (ii) a step of administering any one or a combination of the cancer therapeutic products described herein.

In some embodiments, cancer treatment, vaccination therapy and/or adoptive cell cancer therapy as above described are administered in combination with additional cancer therapies. In some embodiments, cancer treatment, vaccination therapy and/or adoptive cell cancer therapy as above described are administered in combination with targeted therapy, immunotherapy such as immune checkpoint therapy and immune checkpoint inhibitor, co-stimulatory antibodies, chemotherapy and/or radiotherapy.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Co-stimulatory antibodies deliver positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350.

Example of anti-PD-L1 antibodies include, but are not limited to, LY3300054, atezolizumab, durvalumab and avelumab.

Example of anti-CTLA-4 antibodies include, but are not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895.

Example of anti-VISTA antibodies are described in US patent application US20130177557.

Example of inhibitors of the LAG3 receptor are described in US patent U.S. Pat. No. 5,773,578.

Example of KIR inhibitor is IPH4102 targeting KIR3DL2.

As used herein, the term “chemotherapy” has its general meaning in the art and refers to the treatment that consists in administering to the patient a chemotherapeutic agent. A chemotherapeutic entity as used herein refers to an entity which is destructive to a cell, that is the entity reduces the viability of the cell. The chemotherapeutic entity may be a cytotoxic drug. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2—pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP− 16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; biological response modifiers such as IFNa, IL-2, G-CSF and GM-CSF; and pharmaceutically acceptable salts, acids or derivatives of any of the above.

Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

“In combination” may refer to administration of the additional therapy before, at the same time as or after administration of the T cell composition according to the present disclosure.

In addition, or as an alternative to the combination with checkpoint blockade, the T cell composition of the present disclosure may also be genetically modified to render them resistant to immune-checkpoints using gene-editing technologies including but not limited to TALEN and Crispr/Cas. Such methods are known in the art, see e.g.: US20140120622. Gene editing technologies may be used to prevent the expression of immune checkpoints expressed by T cells (see the above listed checkpoint inhibitors) and more particularly but not limited to PD-1, Lag-3, Tim-3, TIGIT, BTLA CTLA-4 and combinations of these. The T cell as discussed here may be modified by any of these methods.

The T cell according to the present disclosure may also be genetically modified to express molecules increasing homing into tumors and or to deliver inflammatory mediators into the tumor microenvironment, including but not limited to cytokines, soluble immune-regulatory receptors and/or ligands.

In some embodiments, a tumor neoantigenic peptide of the present disclosure is used in cancer vaccination therapy in combination with another immunotherapy such as immune checkpoint therapy, more particularly in combination with anti-checkpoint antibodies such as the above exemplified antibodies and notably but not limited to the anti-PD1, anti-PDL1, anti-CTLA-4, anti-TIM-3, anti-LAG3, anti-GITR antibodies.

The present disclosure also encompasses the use of a tumor cell TE signature as defined herein, as a cancer cell biomarker, and/or as a biomarker for immune checkpoint therapy efficacy. In some embodiments, the cancer is glioblastoma and the tumor cell TE-signature comprises SEQ ID NO: 1 to 5020 and is thus a glioblastoma biomarker. In some particular embodiments, the cancer is glioblastoma and the tumor cell TE-signature comprises SEQ ID NO: 1 to 26, 28 to 5020; preferably SEQ ID NO: 1 to 10; 12 to 26, 28 to 430 and 432 to 5020; more preferably SEQ ID NO: 1 to 10, 12 to 26, 28 to 57, 59 to 242, 244 to 255, 257 to 319, 321 to 393, 395 to 430 and 432 to 5020 and is thus a glioblastoma biomarker.

The ability to distinguish self from non-self is a central principle of immunity. Invading pathogens must be recognized as non-self to trigger an adequate response while self-antigens must be tolerated to avoid autoimmunity. Innate detection of pathogens depends on the recognition of pathogen associated molecular patterns (PAMP) by pattern recognition receptors (PRR). Recognition of foreign nucleic acid is a key step in sensing of pathogens, however, host nucleic acid sensors recognise nucleic acid in a non-sequence-specific way. The fact that nucleic acid sensing is not sequence specific blurs the fundamental distinction between self and non-self. Indeed, expression of TEs can generate nucleic acids that act as endogenous PAMPs and possibly drive deleterious immune responses. The triggering of the immune system by an infectious virus antigenically similar to an endogenous retroviral protein could elicit such an autoimmune response. This possibility was also illustrated by the loss of tolerance to an endogenous viral protein in a transgenic mouse after virus infection (see Benihoud K et al., Oncogene (2002) 21, 5593-5600).

Accordingly, there is increasing evidence of a relationship between autoimmune and/or inflammation manifestations and the presence of endogenous or exogenous retroviral sequences. Systemic autoimmune diseases are characterized by defects in immune tolerance to self-antigens, which could include products of endogenous retroviral sequences and retrotransposons. (Herrmann M et al., (1998). Curr. Opin. Rheumatol., 10, 347-354.; Nakagawa K and Harrison L C. (1996). Immunol. Rev., 152, 193-236; C. A. Thomas et al., Cell stem cell 21, 319-331.e318 (2017), Tokuyama, Maria et al. “PNAS vol. 115, 50 (2018): 12565-12572; Zhang X, Zhang R, Yu J., Front Cell Dev Biol. 2020; 8:657. Published 2020 Aug. 7)).

In this context, TE-derived peptides of the present disclosure may be used in tolerance-inducing cellular therapies involving vaccination with or induction of tolerogenic DCs (tolDC) or regulatory T cells (Tregs). Such cellular therapies have indeed gained considerable interest for the treatment and or the prevention of autoimmune diseases (see Flórez-Grau, Georgina et al. “Tolerogenic Dendritic Cells as a Promising Antigen-Specific Therapy in the Treatment of Multiple Sclerosis and Neuromyelitis Optica From Preclinical to Clinical Trials.” Frontiers in immunology vol. 9 1169. 31 May. 2018; and Cauwels, Anje, and Jan Tavernier. “Tolerizing Strategies for the Treatment of Autoimmune Diseases: From ex vivo to in vivo Strategies.” Frontiers in immunology vol. 11 674. 14 May. 2020). Well-suited TE-derived peptides as per the present disclosure include peptides of any one of SEQ ID NO: 3 to 8, 10, 12, 14 to 17, 19 to 21, 24 to 26, 28, 29, 33, 34, 37, 41, 43, 44, 46, 47, 51 to 53, 55, 56, 59, 62, 67, 69, 74, 75, 77, 80, 81, 84 to 87, 90, 92, 96, 97, 99 to 103, 108, 109, 112, 113, 116, 125, 128 to 130, 132, 134 to 137, 140, 142, 145 to 149, 154, to 156, 158, 160, 163, 166, 168 to 171, 174 to 176, 178, 183 to 187, 189, 191, 192, 194 to 197, 200 to 205, 207, 209 to 211, 213, 216, 219 to 221, 224 to 227, 229 to 237, 240 to 242, 247, 249, 250, 252, 254, 255, 258, 261, 263, 264, 266, 268 to 274, 276, 278, 280, 284, 289, 293, 303, 306, 308 to 310, 316, 319, 321, 322, 324, 327, 330, 332, 336, 338 to 342, 345, 347 to 349, 351, 357, 358, 363, 364, 366 and 368 (redundant; see Table 3), notably SEQ ID: 3 to 7, 10, 12, 14 to 17, 19 to 21, 24 to 26, 28, 29, 33, 34, 37, 41, 43, 46, 52 to 53, 55, 56, 59, 62, 69, 74, 75, 77, 80, 92, 97, 99 to 102, 108, 109, 112, 113, 116, 128 to 130, 132, 134 to 137, 142, 145, 146, 148, 149, 154, to 156, 160, 163, 166, 168 to 171, 174 to 176, 178, 183 to 187, 189, 191, 194 to 197, 200 to 205, 207, 209 to 211, 213, 216, 219, 221, 224 to 227, 229 to 237, 240 to 242, 247, 249, 250, 252, 255, 261, 263, 266, 268, 271, 273, 274, 276, 278, 280, 284, 293, 303, 306, 308 to 310, 316, 319, 324, 327, 332, 336, 338 to 342, 345, 348, 349, 357, 358, 363, and 368 (redundant group 1; see Table 3) which are redundantly expressed by numerous TEs in the genome (typically that are encoded by more than 200 genomic TE occurrences). Typically, such TE-encoded peptides are not tumor specific. In some embodiments, the TE-derived peptides are LINE-1 peptides, in particular young L1HS, L1PAx- and L1PBx-derived peptides.

In some embodiments, the expression of one or more TEs (notably encoding the peptides as above mentioned) or preferably a combination thereof can be used as a biomarker for immune disease diagnosis.

### EXAMPLES

### Materials and Methods

### Transposable Elements Annotations

### Classification and TE Metadata

Transposable Elements annotations have been retrieved two different databases: from Homer repeats gtf annotation file (v4.11.1) based on hg19 (v6.4) UCSC annotations; from TEtranscript (Jin et al., 2015, doi: 10.1093/bioinformatics/btv422. Epub 2015 Jul. 23.) hg19 gtf annotation file. Both annotations are based on RepeatMasker database and have been merged based on identical coordinates to obtain following information on each repeat: Class, Family, Subfamily, Divergence, coordinates). L1 family was subdivided into 2 families: (1) L1PA/B/x that include TEs from closely related L1HS, L1PA (x), L1PB(x), L1P(x) subfamilies; (2) Other L1 regrouping all other L1 TEs that are not present in L1PA/B/x. All DNA transposons TEs were classified as DNA. annotatePeaks.pl from Homer was performed to obtain genomic locations (intron, exon, 3′UTR, 5′UTR, intergenic, other) for each individual TE. closest and intersect tools from bedtools (v2.29.2) have been used to retrieved for each TE, distance from closest protein-coding genes from gencode gtf annotation file (Release 19 GRCh37.p13).

### Age of TEs

Repeat age was calculated using percentage of divergence with following formula for human repeats: Divergence/(2.2*10-9), following as the formula from this article (Choudhary et al., Genome Biol, 2020, 21, 16).

### Intact ORFs

Intact open reading frame (ORF) locations were retrieved from gEVE database (Nakagawa, S., and Takahashi, M. U. Database (Oxford) 2016). As analyses were performed on human genome version hg19, hg38 gEVE annotations were formatted and adjusted for hg19 using “Lift Genome annotations” tools from UCSC available here: https://genome.ucsc.edu/cgi-bin/hgLiftOver. Coordinates from intact ORFs from gEVE annotations and from all individual TEs from the genome were matched to assign an intact ORF to individual TEs in case of coordinates overlap. 30517 individual TEs overlapped an intact ORF with most of them being L1 (mostly L1PA/B/x) and ERV (mostly ERV1, ERVK, ERVL) elements. To identify amino acid sequence similarity between canonical TE proteins from gEVE database and peptides from immunopeptidomics results, a blastp was perfomed between gEVE protein sequences and the immunopeptidomics sequences. No threshold on Evalue was set and similarity was estimated and classified in 3 categories: (1) 100% match: no mismatch, no gap and query coverage per HSP to 100%; (2) At most 1 mismatch: 1 mismatch, no gap and query coverage per HSP above 85%; (3) At most 2 mismatches: 2 mismatches, no gap and query coverage per HSP above 85%.

### Retrieving TE Nucleotide Sequence

getfasta (bedtools version 2.30.0) was used to obtain the fasta sequence from each TE. Due to getfasta processing step, first nucleotide is not taken into account, thus the length of sequence is minus 1 nucleotide.

### Analysis of Known TE Proteins

### LTR and LINE Proteins

LTR TEs coding for peptides overlapping an intact ORF were classified as Env, Gag, Pol or Pro using RetroTector annotations from gEVE. For LINE elements, a blastp was performed between LINE-derived peptides and either ORFIp and ORF2p protein sequences found in Uniprot (accession numbers Q9UN81 and 000370). Allowing at most 1 mismatch, 28 hits from either ORFIp and ORF2p were identified among our LINE-derived peptides. LINE and LTR TEs coding for a peptide were also compared to gEVE HMM profile annotations in order to classify the TE protein motif found in those TEs.

### TE ORF Annotations

A homemade R script was used to identify and annotate ORFs from TEs sequence. In details: (1) TE nucleotide sequences were formatted to obtain 6 frames using R package Biostrings (v2.58.0) and its function DNAStringSet and reverseComplement; (2) sequences from 6 frames were translated with translate function from Biostrings; (3) Stop codons and methionine were detected using matchPDict function from Biostrings; (4) Peptides from immunopeptidomics results were also found using matchPDict function; (5) ORFik R package (v1.10.13) was used to detect ORF with at least 30 bp (3 for start codon, 8AA*3 for sequence, 3 for stop codon) and keep only the longest ORF. Two different start codons pattern were submitted to detect ORFs: “ATG” for canonical start codons and “ATG|CTG|GTG|TTG” for canonical and non-canonical start codons. ORFs found only using the second pattern were classified as “CTG|GTG|TTG”; (6) Length of ORFs were calculated using start and end positions; (7) R package ggplot2 was used to represent all identified ORFs, stop codons, methionine and peptides locations in all 5 frames of the TEs.

### Single-Cell Data Analysis

### Downloading Data and Read Alignment to Genome

Smart-seq2 data (GEO accession number: GSE84465) were downloaded from the Sequence Read Archive (SRA) database using prefetch from SRA Toolkit (v2.10.0). SRA files were converted to fastq files using fastq-dump. Fastq files were 75 bp paired-end unstranded reads. Raw RNA reads were mapped to the human genome sequences (hg19) using the 2—pass mode of STAR (version 2.7.1.a) (parameters: —quantMode GeneCounts, —twopassMode Basic, —alignSJDBoverhangMin 1, —bamRemoveDuplicatesType UniqueIdentical, —winAnchorMultimapNmax 1000, —outFilterMultimapNmax 1000, —outFilterScoreMinOverLread 0.33, —outFilterMatchNminOverLread 0.33, —outFilterMismatchNoverLmax 0.04, —outMultimapperOrder Random, —sjdbOverhang 76).

### Quantification of Genes and TE Expression

To compute quantification of TE and gene expression, featureCounts from Subread (v1.6.4) was computed on each genome-mapped reads files. Different parameters were used depending on the analysis: (1) for gene expression: —p—ignoreDup—g gene_id using gencode gtf annotation file; (2) for TEs expression on individual copies (a) with only uniquely mapping reads: —p—ignoreDup—g transcript_id using TEtranscript hg19 gtf annotation file; (b) with uniquely and multi-mapping reads: —p—ignoreDup—g transcript_id —M—primary (3) for TEs expression on subfamilies with uniquely and multi-mapping reads: —p—ignoreDup—g gene_id —M—primary. Cell count files were merged into a matrix with a homemade python script (Python 3.6).

### Filtering Features and Cells, Normalization, Batch Correction

Cell metadata and features raw counts matrices were imported to R (version 4.0.3) to create a SingleCellExperiment R object. CPM, FPKM and TPM values on gene and TE expression were calculated on raw counts prior to any filtering using scuttle R package (v1.0.4) and its functions: calculateCPM, calculateFPKM, calculateTPM. Cells with low number of counts and low number of features (3 times lower than MAD) were removed using Scater and Scran packages. Considering the uniquely-mapped reads TE matrix (1): individual TEs with less than 1 count/cell in average were removed [22000 individual TEs remaining]; for multi-mapped reads (2): individual TEs with less than 5 counts in at least 20 cells were removed to take into account expression in small populations [130028 individual TEs]; for gene expression (3): genes with less than 5 counts in at least 20 cells were removed [19867 genes remaining]; for subfamily expression: no filtering was performed [992 subfamilies]. Raw counts matrices were then normalized using logNormCounts function from scater R package. After several verifications, a batch effect linked to the plate ID of the cells was identified. In order to correct it, removeBatchEffect function from limma R package was used providing the plate ID as batch and the cell type as design.

### Dimensionality Reduction

A single Seurat object was created importing raw, normalized and normalized+corrected features matrices into different assays. CPM, FPKM and TPM matrices were imported as well. Seurat v3 was used for the uniquely mapped reads analysis; Seurat v4 was used for the multi-mapped reads analysis, for the subfamily analysis and the gene analysis. From Seurat, FindVariableFeatures was performed to distinguish the 5000 most variable genes or individual TEs; ScaleData to scale feature expression, RunPCA to compute 75 Principal Components, RunTSNE to perform t-SNE dimension reduction on 50 Principal Components. Dimensionality reduction step was performed on normalized+corrected assay.

### Differential Expression Analysis and Enrichment Tests

From Seurat, FindAllMarkers was performed on annotated cell types with a threshold of 0.25 foldchange (either natural log with Seurat v3 or log 2 with v4) on features expressed in at least 10% of all cells in 1 cell type. Genes, subfamily and individual TE signatures were designed based on FindAllMarkers results using differentially expressed features with an adjusted p-value lower or equal to 0.05. Signature scores were computed with the Seurat function AddModuleScore using the feature signature of interest. This function calculates for each individual cell the average expression of each feature from the signature, subtracted by the aggregated expression of control feature sets. TE subfamily enrichment was performed using all annotated individual TEs in the genome (4.6 million TEs) as a reference and either all expressed TEs or individual TEs signatures from each population as queries. A hypergeometric test was computed using phyper from stats R package (v4.0.3). Then, a False Discovery Rate correction was applied using p.adjust from stats R package.

### FIGURES

Most figures were made using R (v4.0.3). Piecharts, lollipop charts, barplots, violin plots, boxplots, jitterplots, volcano plots, density plots, scatter plots and dimensionality reduction plot were made using either ggplot2 R package (v3.3.3) or functions from Seurat package. Pie donut chart was made with PieDonut function from webR package (v0.1.6). Heatmaps were built with Pheatmap R package (v1.0.12) and ComplexHeatmap (v2.6.2). Clustering method used was ward.D2. IGV (v2.8.10) was used to visualize read coverage of bulk-RNA samples.

### Radarplot and Chromosome Distribution

Radarplots representing feature distribution on chromosomes were made using radarchart function from fmsb R package (v0.7.1). Genomic proportions were calculated using all annotated genes and individual TEs from gencode and TEtranscript annotations respectively.

### Bulk RNA-Seq Data Analysis

### Downloading, Alignment to Genome and Quantification

Around 50 samples from each GTEx tissue were randomly targeted and their fastq reads files were downloaded using prefetch and fasterq-dump from sratoolkit (v2.10.0). Fastq reads from TCGA-GBM project were downloaded using gdc-client (v1.6.1). Alignment and feature quantification (genes, individual TEs, subfamilies) were done in the same protocol described for the Smart-seq2 analysis. Expression was normalized using estimateSizeFactors from DESeq2 R package (v1.30.1) to obtained normalized counts. TPM values were also computed using calculateTPM function from scuttle. Two subsets of TE expression matrices were obtained for each database: (1) Expression matrices with only TEs from the Neoplastic single-cell TE signatures; (2) Expression matrices with only TEs considered expressed. TEs were considered expressed if we could observe at least 5 counts for 20% of the samples (considering separately either all samples from TCGA or GTEx database). 130640 TEs were retained for the TCGA samples whereas 192243 TEs were kept for the GTEx samples. Among those, 103585 TEs were common to both databases.

### Downstream Analysis Ofbulk RNA-Seq Samples

Merged neoplastic signature specific matrix with all samples from TCGA and GTEx was imported in a Seurat object. DESeq2 normalized counts and TPM values were both imported. Using normalized counts, ScaleData, RunPCA and RunUMAP were applied to obtain UMAP representations. To assess signature expression in the samples, mean expression of all TEs from the neoplastic signature was done using TPM values.

### Gene Set Enrichment Analysis

Gene Set Enrichment Analysis (GSEA) was performed using DESeq2 normalized counts matrices of common expressed TEs between TCGA and GTEx databases (103585 TEs) to test enrichment of single-cell neoplastic signature in either Normal or Tumor samples. GSEA (v4.1.0) was running with default parameters. GSEA results were imported to R and ggplot2 was used to made representations.

### Read Coverage

Mapped-reads bam files from neoplastic single cells, immune single cells, TCGA tumor samples and TCGA normal samples were merged using samtools (v1.9) and its merge function. Merged bam files were indexed using index from samtools. Read coverage was calculated on each merged bam file with bamCoverage from deepTools (v3.3.1) and following parameters: —outFileFormat bigwig—normalizeUsing CPM. Results were visualized with IGV (v2.8.10).

### Peptide Binding to HLA-A*02:01, HLA-B*07:02 and Multimer Formation

Predicted peptides were synthetized by GeneCust with a purity >98%. HLA-A*0201 monomers were purchased as easYmers from Immunaware (Copenhagen, Denmark). Predicted and mass-spect (MS) TE-derived Peptides binding to HLA-A*0201 was measured as HLA-I-complex formation by FACS following manufacturer's instructions. Briefly, biotinylated monomers were incubated with synthetic peptides (100 mM) at 18° C. during 48h, then bound to streptavidin-coated beads and stained with PE-conjugated anti-β2-microglobulin. As positive control of HLA-I-complex formation we used CMV peptide pp65 495-503 (NLVPMVATV:: SEQ ID NO: 5021), CMV pp⁶⁵ 417-426 (TPRVTGGGAM:: SEQ ID NO: 5022) and CMV IEl 99-107 (RIKEHMLKK:: SEQ ID NO: 5023) for HLA-A*02:01, HLA-B*07:01. Melan-A mutated sequence (ELAGIGILTV:: SEQ ID NO: 5024), a known good binder peptide to HLA-A*0201, was also included as a second positive control of HLA-I-complex formation for this monomer. Binding is represented as percentage of HLA-I-complex formation relative to CMV positive control. Peptides with HLA-I-complex formation of at least 50% relative to positive control were used in in-vitro vaccinations experiments.

For multimer formation, peptide-HLA-I-complexes were tetramerized using different combinations of streptavidin conjugated to fluorochromes (PE, APC BV421, BV711, PE-CF549 and PECy5) in a final concentration of 8 mg/ml. All tetramers were kept at 4° C. and used within 2 months.

### Multimer Staining and Analysis

Multimer staining was performed on total cells after in-vitro vaccination experiments by combining 1 μl of each tetramer specificity and two different SA-labelled tetramers per specificity. The staining was performed during 20 min at RT in a final volume of 100 μl of PBS 1% BSA/1M cells. Then, 100 μl of surface antibody mix containing anti-CD3 BV650 and anti-CD8 PECy7 (BD Biosciences) was added at 1/200 final dilution and incubated for further 20 min at 4° C. Finally, cells were washed twice with PBS-1% BSA and analyzed by flow cytometry. Live/Dead Aqua-405 nm (ThermoFisher) was used to exclude dead cells. Data was collected using a ZE5 Cell Analyzer (Bio-Rad) and analyzed using FlowJo v10.3.

Multimer analysis was done on live, single cells, CD3+CD8+ cells following the strategy described by Andersen et al. (Andersen et al., Nat Protoc, 2012, 7, 891-902). Expansions are considered positive using the double multimer staining criteria. Expanded populations for each peptide are represented either as frequencies of total CD8+ cells in each replicate or as total multimer frequencies among total CD8+ T cells evaluated in all replicated for one donor.

### In-Vitro Vaccinations Assay

Buffy coats from healthy donors were obtained from Etablissement Frangais du Sang (Paris, France) in accordance with INSERM ethical guidelines. According to French Public Health Law (art L 1121-1-1, art L 1121-1-2), written consent and IRB approval are not required for human non-interventional studies.

PBMCs were obtained by density gradient separation using Lymphprep (StemCell technologies) and phenotyped by FACS using anti-HLA-A2 antibodies (clone BB7.2, BD Biosciences) and anti-HLA-B7 antibodies (clone BB7.1, Biolegend). Only HLA-A2+ and HLA-B7+ donors were used. Monocytes and lymphocytes from the same donor were purified as CD14⁺, CD4+ and CD8⁺ cells by positive selection using magnetic beads (Miltenyi Biotec). Monocyte-derived dendritic cells (mo-DCs) were obtained by differentiation of CD14+ fraction during 5 days at 10⁶ cells/ml in RPMI-1650/Glutamax (Gibco), 10% FBS, penicillin (100 U/ml)/streptomycin (100 μg/ml) supplemented with recombinant human IL-4 (50 ng/mL) and GM-CSF (10 ng/mL). Isolated CD4⁺ and CD8⁺ T cells were cryopreserved during mo-DCs differentiation.

After differentiation, mo-DCs were seeded in culture medium in 24 well plates at 1×10⁶ cells/ml and maturated OVN with LPS (100 ng/ml). After that, culture media was removed and LPS treated mo-DCs were pulsed during 3 h at 37° C. with a mix of selected good-binder TE-derived peptides (either predicted or MS-derived from HLA-I peptidomics data). Each peptide was at 1 g/mL final concentration. Finally, peptide-loaded mo-DCs were harvested, pelleted and counted. Cryopreserved lymphocyte fractions were thawed and co-cultures were performed by mixing 1×10⁶ CD8⁺ T cells with 0.1×10⁶ CD4⁺ T cells and 0.1×10⁶ peptide-loaded mo-DCs (CD8-CD4-mo-DCs ratio: 10:1:1, respectively) in a final volume of 2 ml in 24 well plate. Each well was considered as an independent replicate. Total number of replicated was determined by the total number of CD8⁺ T cells. Without disturbing the cells, media was half-changed after 5 days and then monitored every 3 days until day 15-20. Expansion of specific CD8⁺ T cells populations were evaluated by FACS using multimer staining. X-vivo 15 media (Lonza) supplemented with penicillin (100 U/ml)/streptomycin (100 μg/ml) (Gibco), 10% FBS, 10 U/ml of IL-2 (Novartis) and 10 ng/ml of IL-7 (PeproTech) were used as culture media. As negative control, with MS-derived peptides, a replicate using mo-DCs non-peptide pulsed was included. For HLA-A2⁺ donors a positive control of T-cells expansions (1 or 2 replicates) using mo-DCs pulsed only with Melan-A peptide (ELAGIGILTV) was included. Within the mix of MS-derived HLA-A2⁺ peptides, 3 HLA-A*02:01 binding peptides derived from the canonical sequences of normal proteins (present in Uniprot normal proteome) were included.

### Mass Spectrometry Based Immunopeptidomics

### Mass Spectrometry Data Analysis

Mass spectrometry-based immunopeptidomics files were obtained from PXD020079, PXD008127, PXD003790 and MSV000084442 and analysed with ProteomeDiscoverer 2.5 (ThermoFisher) using the following parameters: no-enzyme, precursor mass tolerance 20 ppm and fragment mass tolerance 0.02 Da. Methionine and N-acetylation were enabled as variable modifications. Using Percolator, a false discovery rate (FDR) of 1% was applied at peptide level and no FDR was used at protein level. Spectra were searched against the human Uniprot/SwissProt with isoforms (updated Jun. 3, 2020) concatenated with the 6 reading frame in silico translated neoplastic enriched TE database. Identified potential TE-derived peptides were filtered afterwards with UniProt/TrEMBL database considering leucine-isoleucine and lysine-glutamine as equivalent, respectively. Finally, spectrums from identified TE-derived peptides were manually verified.

### Peptide Hydrophobicity Index (HI) Calculation

For retention time versus hydrophobicity comparisons, HI were predicted using SSRCalc (Krokhin et al. 2004) web server (http://hs2.proteome.ca/SSRCalc/SSRCalcX.html).

### Single and all Assignments Definition

As multiple TEs can code for the same peptides, two different categories were made in order to make observations on TE-encoding peptides features. All assignments correspond to all TEs coding for a peptide (all 568 TEs for 370 peptides). Single assignment corresponds to a random selection for each peptide of an individual TE that can encode the corresponding peptide (370 TEs for 370 peptides).

### Identifying Potential Peptide-Encoding TEs

In order to identify or screen all TEs incorporating peptide sequences, peptides sequences were aligned to all annotated individual TEs in the genome in all six frames using tblastn (v2.11.0+). Sequences from all TEs in the genome were retrieved using getfasta from bedtools (v2.30.0) using TETranscript gtf processed into BED format. No restriction on Evalue was requested.No restriction on Evalue was requested. All hits with a number of mismatches equal to 0, a number of gap openings equal to 0 and a query coverage per HSP of 100 were kept and considered as peptide-coding TEs in addition to those from the neoplastic signature identified with ProteomeDiscoverer.

Spectrum Validation with Synthetic Peptides

To validate the spectra, 24 of the identified peptides were synthesized (GeneCust) with an HPLC purity of 95% and were injected in a Velos Orbitrap (CID). Raw files were analysed with ProteomeDiscoverer 2.5 (ThermoFisher). Spectrums were exported and compared to the spectra derived from the immunopeptidomics analysis. Only PSM with the same charge between synthetic and endogenous and without modifications were analysed. The same fragmentation type (CID or HCD) between both spectrums was prioritized when possible

### Identification of Tumor-Enriched TE-Derived Peptides

TPM expression of all possible TEs from the genome that can potentially code for the identified peptides was retrieved and 90^{th} percentile values were calculated for each tissue. TEs coding for each specific peptide were selected and their 90^{th} percentile values were summed to obtain the total transcript expression related to these peptides. For non-redundant peptides, related transcript expression was directly the 90^{th} percentile value of the TE coding for the peptides. A log 2 ratio was then performed between peptide related expression in GBM samples compared to each GTEx tissue to assess if the related expression of these peptides were higher in GBM samples compared Normal tissues. Using median TPM expression in GBM samples as a threshold, the percentage of expression in Normal samples with an equal or higher expression was also calculated for each tissue. Pheatmap function from ComplexHeatmap R package (v2.6.2) was then used to represent the log 2 ratio, the 90^{th} percentile values as well as the percentage of expression in Normal samples. Clustering method used in the heatmap with the log 2 ratio was ward.D2.

### Statistical Analyses

Wilcoxon tests were performed with R package ggpubr (version 0.4.0) and its function stat_compare_means (1): to compare distance to closest gene between Immune and Neoplastic signatures (2) to compare mean expression of the neoplastic signature in bulk RNA-seq samples; (3) to compare length of canonical and non-canonical TE-derived peptides ORFs. Pearson correlation scores were computed using stat_cor from ggpubr: (1) to assess the correlation between TEs and their closest protein-coding gene; (2) to assess the correlation between median age of TEs coding for a peptide and the number of TEs that can code for the peptide. Two proportions z-test were computed to compare LINE proportions in different subsets of individual TEs. The corresponding p-values to symbols are as follows: ns: p>0.05; *: p<=0.05; **: p<=0.01; ***: p<=0.001; ****: p<=0.0001.

### Results

### Single Cell TE-Expression Resolves all Cell Populations in Tumors

It was reasoned that a powerful way to identify TEs expressed specifically in tumor cells would be to compare TE expression in tumor and in tumor-infiltrating cells from the same patients. To do so, single cell transcriptomics (scRNAseq) of all cells present in the tumor microenvironment were used. The study was initiated on a public data set including tumor and juxta-tumor samples from 4 GBM patients analyzed by SMARTseq2 (Darmanis et al., Cell Rep, 2017, 21, 1399-1410). Consistent with the analysis performed in the original article, dimensionality reduction and t-SNE visualization based on gene expression resolves the 7 sorted cell populations from the tumor core and the surrounding tissue: immune cells (mostly macrophages), neoplastic cells and oligodendrocyte precursor cells (OPCs) are the most numerous (FIG. 1B.

To investigate TE expression in single cells, scRNAseq reads were mapped to either TE subfamilies (as shown previously in Kong et al., Nat Commun, 2019, 10, 5228) or to individual genomic TEs (FIG. 1A). Because mapping of TEs to individual genomic locations can be affected by high conservation of their repeat motifs, especially in young TE subfamilies, the use of uniquely and multi-mapping RNAseq reads were compared. Uniquely mapping reads allow accurate estimation of the expression of older TE subfamilies, but underestimates the expression for youngest TE subfamilies, as compared to multi-mapping reads, which reflect more accurately expression of young TE subfamilies (Lanciano and Cristofari, Nat Rev Genet, 2020, 21, 721-736). To quantify TE expression, FeatureCounts with—primary and randomly-reported positions (−M, for multiple alignment) were used as recommended in Teissandier et al. (Teissandier et al., Mob DNA, 2019, 10, 52)). tSNE based on expression of 992 TE subfamilies, or 5000 most variable individual TEs in single cells, like gene expression, resolves all cell populations in the tumor microenvironment (FIG. 1B middle panel). Neoplastic cells and OPCs are mostly present in tumor and juxta-tumor samples, respectively, while, as expected, immune cells are present in both (Darmanis et al., 2017). Individually mapped TEs allow better resolution of the different cell populations than TE subfamilies (FIG. 1B right panel). These results show that expression of individual TEs can be resolved at the single cell level and is sufficient to distinguish different cell populations in the tumor microenvironment.

### Te Subfamilies are Differentially Expressed in Neoplastic and Immune Cells

To better understand the nature of these TEs, differential expression (DE) analyses of TEs in each cell population were performed against all others, thus defining population-specific TE signatures. These signatures are highly specific for neoplastic cells (Table 2), immune cells (FIG. 1C), and for each of the other cell populations present in the tumor microenvironment. Heatmap representation of unsupervised clustering of the 20 most differentially expressed TEs for each type of cells based on the average log 2 fold change shows selective expression in each cell population, including in neoplastic cells (not shown). To further investigate the nature of the TEs differentially expressed in each cell population, each signature to all TEs expressed in the data set (130,028) was compared. TEs differentially expressed in neoplastic cells are depleted in SINEs (51.68% vs. 44.52%) and enriched in LTRs (8.33% vs. 12.11%), while TEs in immune cells are depleted in LINEs (30.29% vs. 26.47%) and LTRs (8.33% vs. 5.62%) and enriched in SINEs (51.68% vs. 59.18%), confirming the results from direct mapping of TE subfamilies. Statistical analyses by subfamily show strong enrichment for several LTR subfamilies in neoplastic cells (mainly HERV), while immune cells differentially express several SINE subfamilies (mainly Alu) (FIG. 1D). The different cell types present in the tumor environment therefore express distinct patterns of TE subfamilies that can be analyzed from individually mapped TEs by single cell transcriptomics.

The relationship between TE expression and genomic copy number alterations has been next investigated. Gain of chromosome 7 and loss of chromosome 10 are recurrent events in GBM (Kurscheid et al., Genome Biol, 2015, 16, 16.). Genes and TEs were mapped in each cell type-specific signature to their respective chromosomes. As shown in FIG. 1E, TEs differentially expressed in neoplastic cells, but not in other cell populations, present a clear bias for chromosome 7 (FIG. 1E and FIG. 1F). The bias for chromosome 7 in neoplastic cells is even stronger for TEs than for genes (17, 91% of expressed TEs are encoded in chromosome 7, compared to 9.14% for genes) (FIG. 1F). The loss of chromosome 10, by contrast, is similar in the TE (0.93% vs. 4.55% in the genome) and gene signatures (1.43 vs. 3.88% in the genome) (FIG. 1F). Individual TEs can therefore be accurately mapped from scRNAseq and, as expected, show a chromosome 7 bias selectively in neoplastic GBM cells.

TE Expression in Neoplastic Cells is Enriched in Elements Independent of their Closest Gene

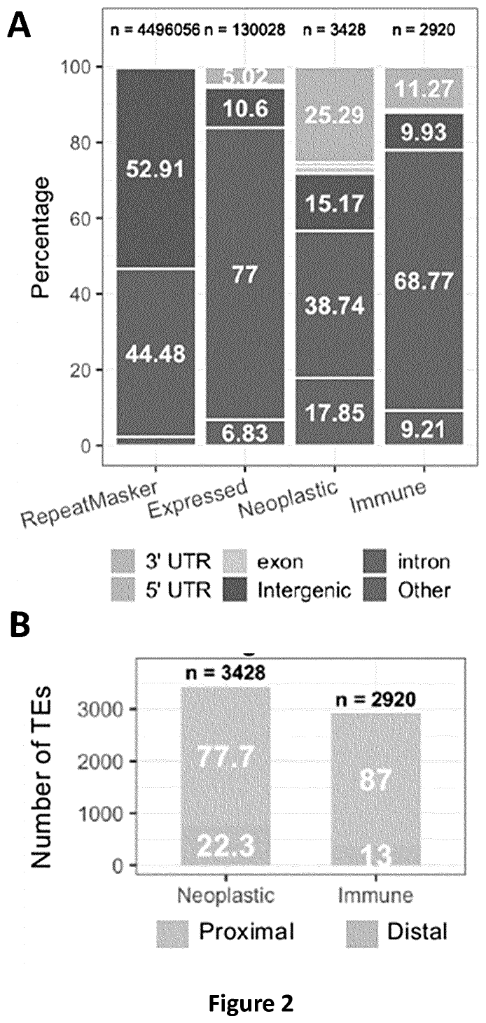

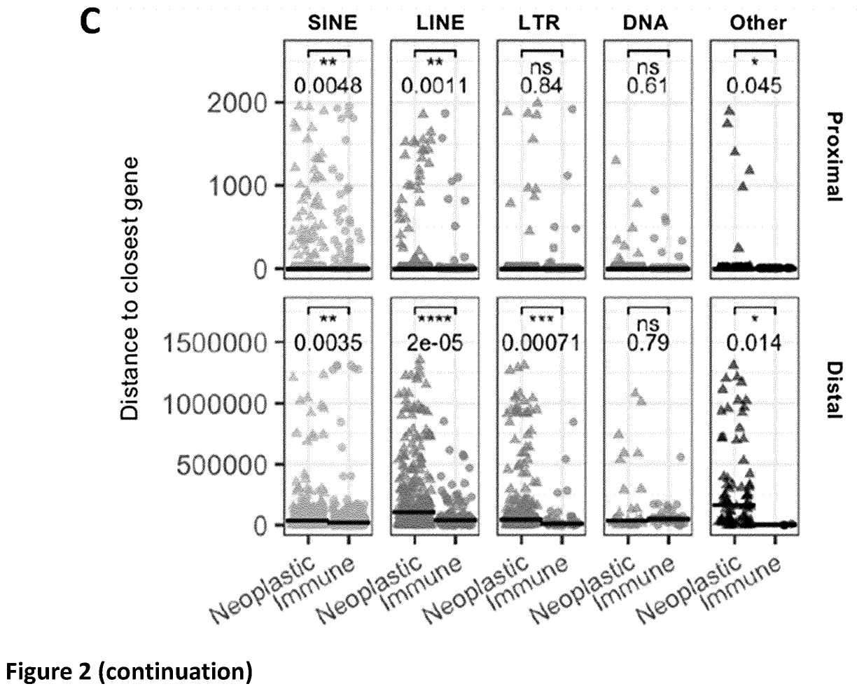

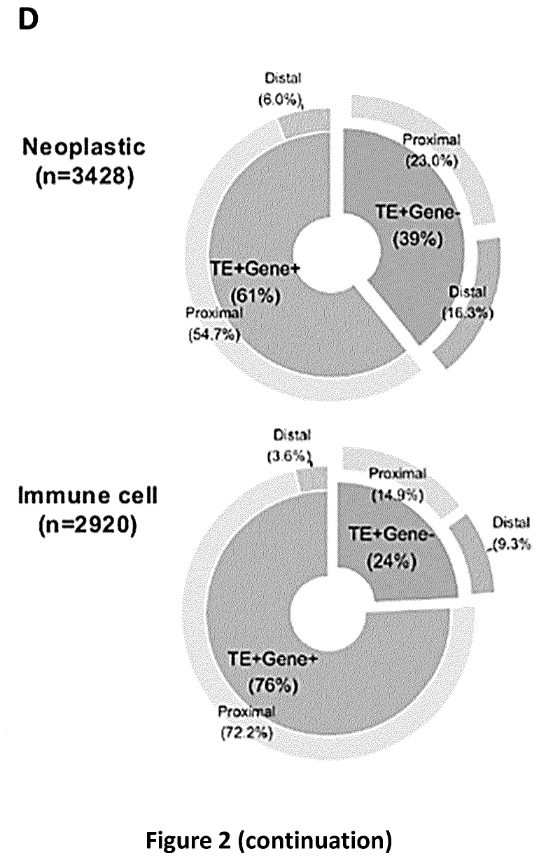

To better understand the control of TE expression in different cell populations, TE genomic locations were first analyzed. As compared to all expressed TEs in the data set, TEs differentially expressed in neoplastic cells show reduced intronic locations (77% vs. 38.74%), including when compared to the proportion of intronic TEs differentially expressed in immune cells (68.77%) (FIG. 2A). Neoplastic TEs also show a marked increase in 3′UTR encoded TEs (25.29%), compared to all expressed TEs (5.02%) or to immune cell TEs (11.27%) (FIG. 2A). These results show that, while TEs differentially expressed in immune cells are largely intronic, in neoplastic cells intergenic and 3′UTRs TEs are more frequently differentially expressed.

Consistent with these results, the proportion of TEs located at more than 2 Kb (distal) from the nearest protein-coding gene is higher in the neoplastic cell signature (22.32%) that in the immune cell signature (12.98%, FIG. 2B). t-SNE analysis based on distal TEs resolves all cell populations, suggesting that cell type-specific TE expression may not be exclusively due to gene-driven transcription. Consistently, the TE-gene distances are increased for TEs differentially expressed in neoplastic cells, especially for LINE and LTRs (FIG. 2C), as compared to those TEs differentially expressed in immune cells. Higher distances from the closest genes for TEs expressed selectively in neoplastic cells could reflect gene-independent TE expression, including enhancer-dependent or long non-coding RNA (Lnc) RNA-dependent read-through transcription. The correlation between expression of TEs and their closest genes, in neoplastic and immune single cells was therefore next analyzed. Quantification of the proportions of proximal and distal TEs, expressed together or independently of their closest gene, shows that the proportion of both proximal and distal TEs that are expressed while their closest gene is silent (TE+ gene−), is higher in the neoplastic cell (39%) than in the immune cell TE signature (24%) (FIG. 2D). These results show that higher proportions of TEs differentially expressed in neoplastic cells are distant and transcribed independently of their closest gene neighbor, suggesting a higher level of autonomy in TE transcription in GBM cells.

### Validation of the Single Cell Neoplastic TE Signature in an Independent Cohort of GBM Patients

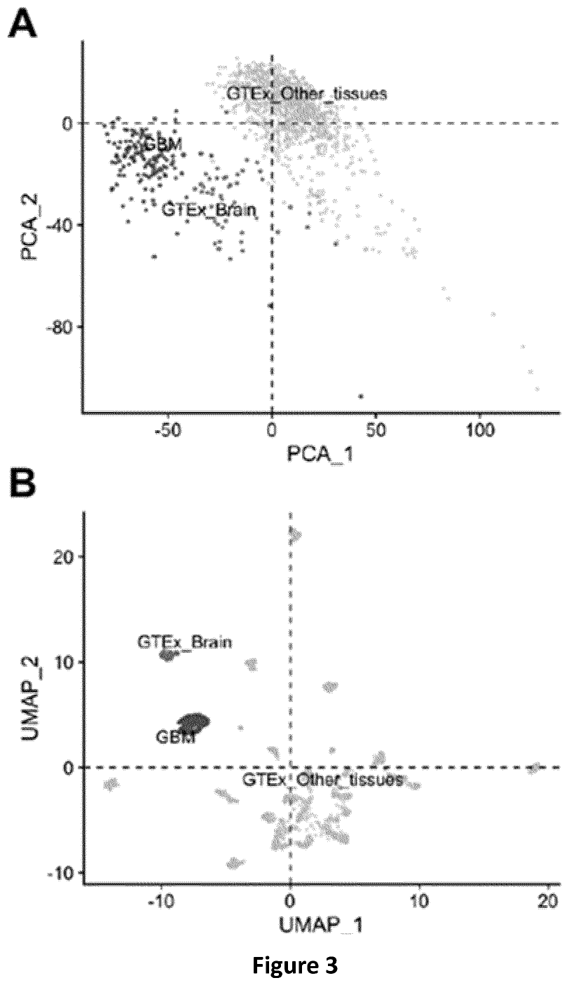

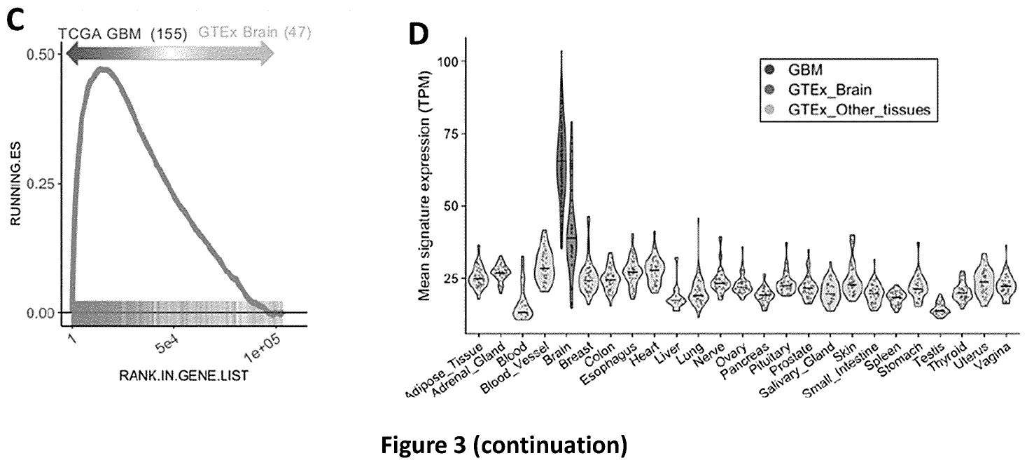

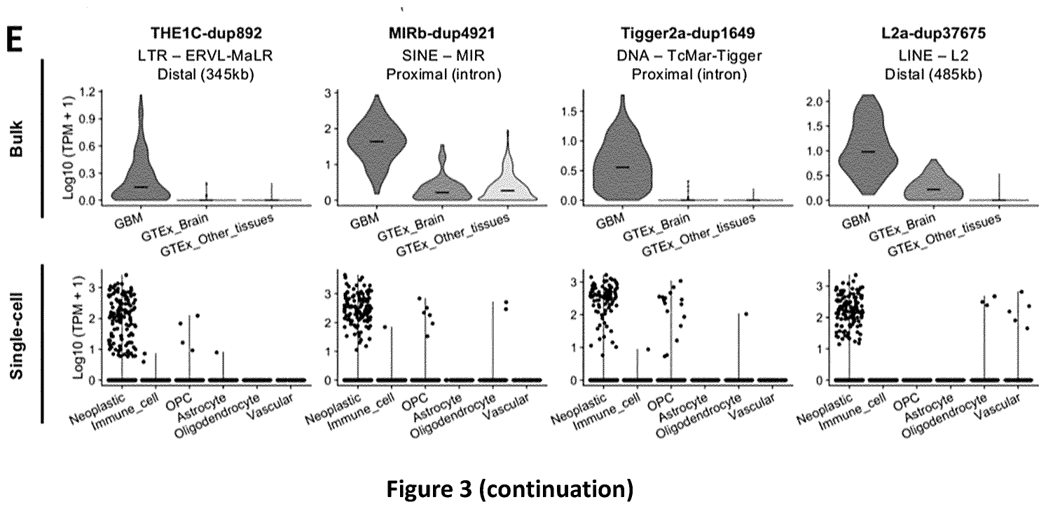

To validate the single cell-based TE-signatures, bulk RNAseq from the TCGA (155 GBM patients and 5 juxta tumor samples) and GTEx (1080 healthy samples from 25 tissues) was next analyzed. The muscle GTEX cohort was exclude because the library size is smaller compared to other. RNAseq reads were mapped to human genome and TE expression was quantified using RepeatMasker annotations. Principal component analysis (PCA) and Uniform Manifold Approximation and Projection (UMAP) based on GBM TE-signature show that GBM samples cluster away from normal tissue GTEx samples (FIGS. 3A and 3B). Heatmap Z-score representation in TCGA and GTEx samples shows higher expression of the 2000 top TEs of the single cell GBM signature in TCGA GBM samples, and reduced expression in healthy tissues (not shown). Gene Set Enrichment Analysis (GSEA) analysis shows that expression of the scRNAseq GBM TE-signature is highly enriched in GBM vs. normal brain samples (NES=1.67 and FDR<0.05, FIG. 3C) and vs. other normal tissues samples in GTEx. The mean scRNAseq GBM TE-signature expression level is also higher in GBM samples, compared to normal tissue GTEx samples (FIG. 3D). Of note a fraction of healthy brain tissue samples express high levels of the GBM TE-signature. Examples of individual TEs overexpressed in both datasets, bulkRNAseq (FIG. 3E, top panels) and scRNAseq (low panels) illustrates the specific expression of certain TEs in GBM cells. Analysis of individual TEs from scRNAseq is thus accurate and allows the identification of recurrent, tumor-specific TEs.

### TE-Derived Peptides are Presented on HLA-I and are Immunogenic In Vitro

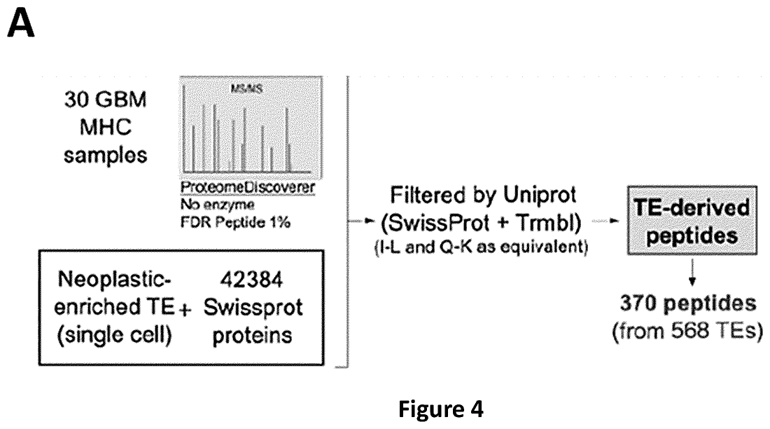

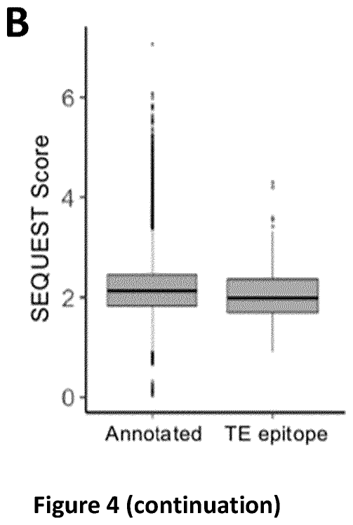

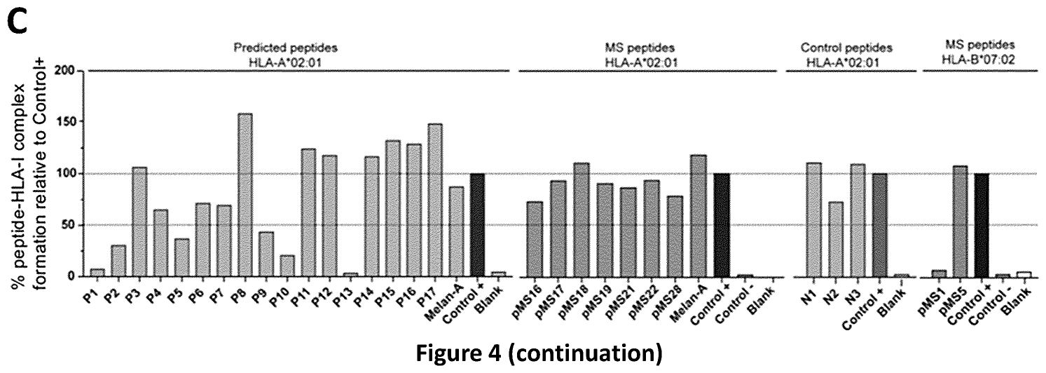

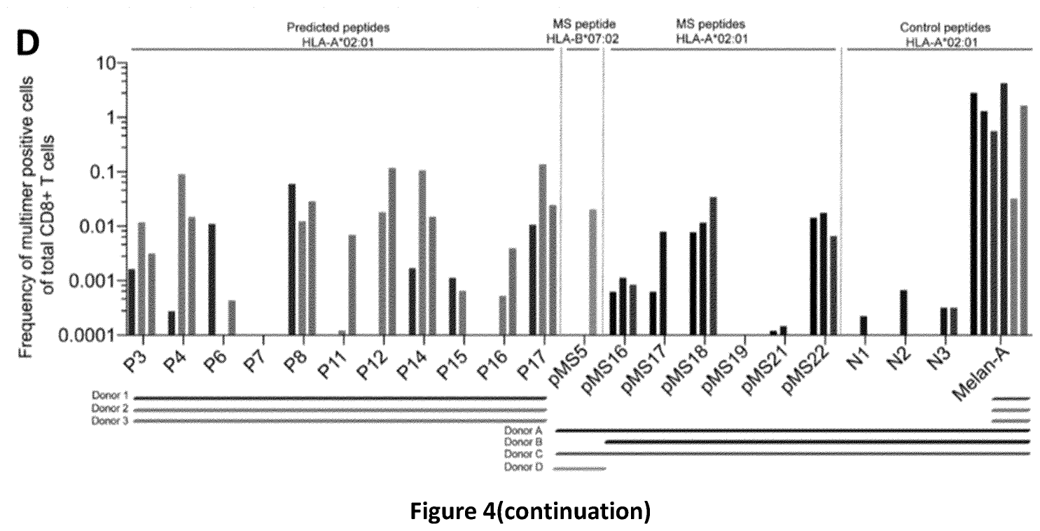

To investigate if TE-derived peptides are presented by HLA-I molecules in GBM cells, 30 mass spectrometry-based immunopeptidomic samples from GBM primary tumors and cell lines (Forlani et al., Mol Cell Proteomics, 2021, 20, 100032; Sarkizova et al., Nat Biotechno, 2020, 38, 199-209; Shraibman et al., Mol Cell Proteomics, 2018, 17, 2132-2145; Shraibman et al., Mol Cell Proteomics, 2016, 15, 3058-3070) (FIG. 4A) were used. Two different databases of in silico translated TEs were generated from multi-mapping (3428) or uniquely-mapping (1945) differentially expressed, TE-encoding, reads (. Sequences of all TEs were in silico translated in all 6 reading frames (sense and anti-sense). The resulting translated TE sequences were combined with the human annotated proteome and interrogated in HLA-I peptidomics samples using Proteome Discoverer. The identified TE-derived peptides were then filtered against canonical proteins (Swissprot+TrEMBL) and spectra were reviewed manually (FIG. 4A). From 178 to 13720 total peptides were identified per sample from which 370 were TE-derived peptides (Table 3), including 63 peptides predicted from both signatures, 147 only from the multimapped-read, and 160 only from the uniquely-mapping read signatures. Heatmap representation of all identified TE-derived peptides shows that the number of peptides varies among samples, and that the same peptides are found recurrently in several different patients and cell lines (not shown).

TE-derived peptides showed similar SEQUEST quality scores and peptide length distribution as Uniprot-annotated peptidome, indicating that they are reliable identifications (FIG. 4B). HLA-A3 binding TE-derived peptides (n=96) contained the expected binding motif obtained from Immune Epitope Database (IEDB) (not shown). In addition, TE-derived peptides maintained the correlation between hydrophobicity and retention time (not shown). These results indicate that TE-derived peptidome is reliable and contains similar characteristics to the canonical peptidome. Twenty-three TE-derived peptides were synthetised and validated by comparison d with the endogenous sequence (out of 24 tested). Confirming the robustness of the pipeline, the identified peptides (using both the unique and multi-mapping signatures), similar to the TE signatures, are preferentially encoded by TEs from chromosome 7. TEs differentially expressed in GBM neoplastic cell are thus a source of peptides presented on HLA-I molecules.

To investigate the possibility that TE-encoded peptides can represent can encode potential tumor antigens, T cell precursors were searched in healthy donors. The TEs differentially expressed in neoplastic cells were in silico translated and NetMHC was used to predict HLA-A2 binding peptides (strong and weak binders). TEs were selected based on p-value (less than 1e⁻⁵⁰) and average log fold change (higher than 2.5) in the differential analysis. Using a tetramer-forming assay, the binding of 7 peptides from immunopeptidomics and 17 from NetMHC predictions on in silico translated GBM T-signature for HLA-A*02:01 (and for HLA-B*07:02 (2 peptides from the immunopeptidomics)) was first experimentally tested (FIG. 4C). 19 peptides were confirmed as HLA-I binders and were used to test immunogenicity in vitro. Immunogenicity was tested by co-culturing peptide-loaded monocyte-derived dendritic cells with autologous CD4⁺ and CD8⁺ T cells from 7 healthy donors and tetramer staining was used as read-out. Mutated Melan-A peptide, a strong binder to HLA-A*02:01 and high T cell precursor frequency in most healthy donors (Pittet et al., J Exp Med 190, 1999, 705-715) was used as positive control for cells expansions. 3 HLA-A*02:01 binding peptides from proteins not expressed specifically in GBM tumors and derived from canonical proteome, were also included as negative control. Expanded tetramer-positive CD8⁺ T cells were observed for 14 TE-derived peptides (including 5 from the immunopeptidomic identifications; Table 4), in at least one donor. The 3 peptides derived from canonical proteins induced very weak or no responses, although Melan-A derived peptides (also a non-TE-derived non-GBM-specific protein) induced high T cell responses (FIG. 4D). In conclusion, a subgroup of TEs differentially expressed in GBM can encode HLA-I-binding peptides that are immunogenic in vitro in healthy donors and could potentially represent a source of tumor antigens.

### Li, LTR and SVA Subfamilies are Main Source of TE-Derived HLA-I Peptides

To investigate the nature of the tumor-enriched TEs that encode HLA-I-presented peptides in GBM, next the peptide sequences to all differentially expressed TE from the single cell GBM TE-signature, was mapped. In doing so, it was realized that although 85.41% of the 347 peptides are encoded by one single TE, the remaining 15% of peptides could potentially be encoded by 2 to hundreds of TEs among those differentially expressed in this GBM-TE signature (FIG. 4A). These peptides will be referred to as “single-TE encoded peptides” or “multi-TE encoded peptides”. For further analyses, when the same peptide can be redundantly encoded by multiple TEs (since which TE encodes the peptide cannot be determined), it was considered either all the TEs bearing the peptide-coding nucleotide sequence (“all assignments”), or only one (chosen arbitrary) of these TEs per peptide (“single assignment”). The genomic location of the peptide-coding TEs relative to the nearest gene was first analyzed.

Among TEs coding for HLA-I-presented peptides 37.85% and 31.89% (for all and single assignments, respectively) are distal (over 2 Kb from their nearest gene), as compared to all expressed TEs (12.11%) or to neoplastic differentially expressed TEs (22.32%). Analysis of the genomic locations of peptide-coding TEs revealed that that most are intergenic (35.04% and 28.92% for all and single assignments, respectively, compared to 15.17% in the GBM-TE signature). The proportion of intronic TEs is also increased, but not as much (50% and 50.7% for all and single assignments, respectively, compared to 38.74% in TEs expressed in neoplastic cells). 3′ UTR TEs are less frequent in peptide-coding TEs 25.29% of TEs in neoplastic differentially expressed TEs, and only 5.81% and 7.03% for all and single assignments, respectively, among peptide-encoding TEs. These results establish selectivity in the genomic location of peptide-encoding TEs, which are preferentially intergenic or intronic, and not found in 3′UTRs.

It was then investigated if the identified peptides are preferentially derived from certain TE classes. Based on both all and single assignments, peptide-encoding TEs are significantly enriched for LINE elements (which represent around 30% of all expressed or neoplastic differentially expressed TEs, and from 52 to 64%, for all and single assignments of peptide-encoding TEs, respectively). These TE class analyses also revealed that TEs classified as “others” are also enriched (see below). These TE class analyses also revealed that TEs classified as “Other” are also enriched (see below). Among the “Other” category, SVA elements and other types of repeats codified in RepeatMasker as RC, RNA, Satellite and Unknown are represented. Among all TE-derived peptides from this category, around half of them are from SVA elements (23 out 51). Regarding SINE elements, it was observed that they are depleted among peptide-generating TEs (from 51.68% and 44.52% in all expressed and differentially expressed TEs, to around 11% in TE-encoding peptides). Therefore, GBM differentially expressed LINE elements are a major source of TE-derived peptides presented on HLA-I in GBM.

TEs within each class are classified in families and subfamilies. The evolutionary “age” of these subfamilies can be estimated from the degeneration of their characteristic repeat motifs (Choudhary et al., Genome Biol, 2020, 21, 16). A few of the most recent subfamilies include TEs that encoded for intact viral protein ORFs and some of which can still be “active” in terms of retro-transposition (Burns, Science, 2017, 348, 803-808; Rodic et al., Nat Med, 2015, 21, 1060-1064; Scott et al., Genome Res, 201, 26, 745-755). This finding that certain peptides can be redundantly encoded by multiple TEs could be due to conserved sequences present in young from the same TE subfamilies. Therefore the ages of the TE subfamilies was analyzed for each peptide-encoding TE. The median age of the peptide-coding SINE and DNA TEs are similar to all genomic TEs annotated in RepeatMasker, and to all expressed and differentially expressed TEs. For LTRs, the proportion of younger TEs is increased among peptide encoding TEs (decreasing the median age of the peptide-encoding TEs compared to other categories), but older TEs are also presented on HLA-I. For LINE and “others” (see below a more detailed analysis of this category) classes, a bi-modal distribution is observed, with a clear enrichment in peptides encoded by TEs from young subfamilies (under 50 M years) that are rare in RepeatMasker, in all expressed and in neoplastic differentially expressed TEs. Thus, among LINE, and LTR TE classes, recent TEs are more prone to provide peptides for HLA-I presentation.

### Ancient Viral Proteins are a Source of HLA-Presented Peptides

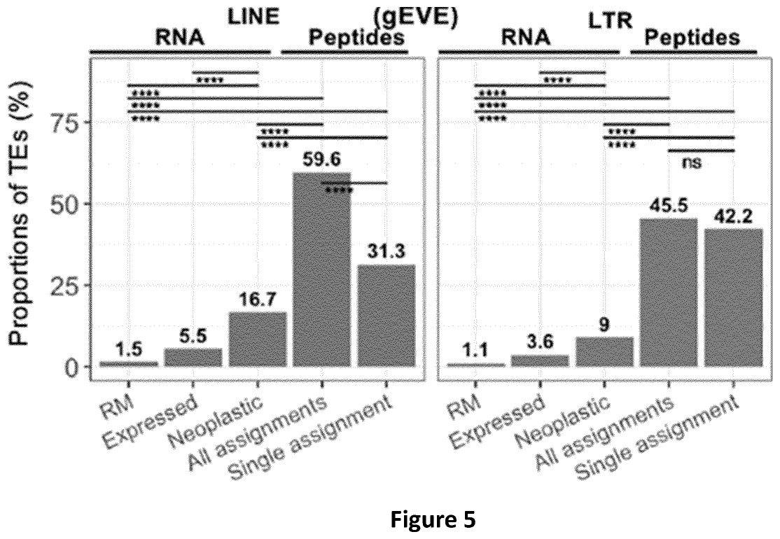

It was next investigated if peptides from TEs are derived from annotated Endogenous Viral Elements (EVE) which are documented and validated in the gEVE database (Nakagawa and Takahashi, Database (Oxford) 2016). These EVEs of at least 80 amino acids were identified processing both RepeatMasker annotations and conserved known motifs from viral proteins like Gag and Pol. Mapping peptide-coding TEs to gEVE shows that, for both LINEs and LTRs, TEs mapping annotated EVE are significantly enriched among peptide-coding TEs (based on both all and single assignments), as compared to RepeatMasker, all expressed and differentially expressed TEs (FIG. 5). Consistent with these results, mapping of the peptide-coding TEs to their corresponding sub-families shows selectivity for Alu among SINEs, L1PA/B/x and L2 among LINEs, ERV1, ERVK, ERVL and ERV-MaLR among LTRs and SVA among others. Allowing one or two nucleotide mismatches (to take into account possible mutations or polymorphisms) increases markedly the proportion of peptide-coding TEs that map to annotated ORFs from gEVE, including for classes and sub-families, suggesting that recently mutated TEs are also a major source of peptides for HLA-I presentation. Most peptides are derived from ORFs bearing a start codon, either ATG (canonical) or CTG/GTG/TTG (non-canonical).

An example of peptides are 3 peptides encoded in a SVA-family member, SVA_B_dup189. The 3 peptides are encoded on the forward strand, in 2 different reading frames (RF). The 2 peptides encoded in RF1 are present in ORFs longer than 30 amino acids, while the third peptide (encoded in RF3) is not found in a detected ORF. It could be that the ORF is shorter than 30 amino acids, that the start codon for this ORF is not among the 4 ORFs used in the pipeline or that the start codon is outside the TE. Analysis of the length of the ORFs encoding HLA-presented peptides shows that among L1PA|B|x, but not among other TE subfamilies, ORFs generating peptides and containing a canonical ATG start codon are longer than the ones starting with a non-canonical one. Among peptide-coding TEs mapping a gEVE annotate LTR ORFs, the actual peptide coding sequence can be present in all retroviral proteins, with an enrichment for Gag (which represent 10.6% of ORFs in gEVE, vs. 28% in peptide-coding TE ORFs). In the case of LINEs, Pol are the only gEVE annotated proteins. Blast of the peptide-coding sequences shows that the majority of LINE encoded peptides are not derived from the two major LINE ORFs, ORF1p (3.1%) and ORF2p, (10.8%). In conclusion, TEs from young subfamilies, preferentially bearing retroviral protein motif, are more prone to provide peptides for presentation by HLA-I molecules in GBM cells. The peptides are encoded by ORFs bearing canonical or alternative start codons and can be from 10 to 1000 amino acids long.

### TE Subfamilies Share HLA-Presented Peptide Coding Sequences

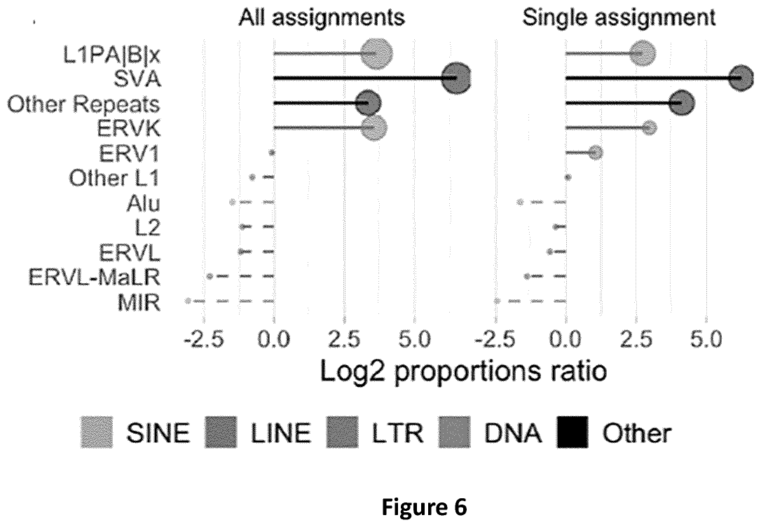

To investigate if some TEs are more prone to provide HLA-binding peptides than others, the proportions of TE families among the ones differentially expressed in GBM (and used for the peptide MS/MS search) and the proportions found among the TEs that code for peptides were compared. For LTRs, SINEs and Others, the proportions of different families are similar in the GBM TE-signature and the peptide encoding TE (both with all or single assignments). For LINEs, in contrast, peptides are preferentially derived from L1PA/B/x: 25.3% in GBM TE-signature vs. 76.6% or 49.7% for All and Single assignments, respectively. Other LINE families are depleted among peptide-coding TEs (especially L2, which represent 25.1% of GBM TE-signature and provide for only 7.4% or 15.4% of peptide-coding TEs, with all and main assignments, respectively). Statistical analysis shows significant enrichment in peptide-coding TE over GBM TE-signature for L1PA/B/x and SVA, as well as in ERVK. Among the RM category “Others”, XX are also enriched. L2, SINEs (including Alu and MIR) and ERVs (including ERVL and ERVL-MalR) are all significantly depleted among peptide-coding TEs, as compared to GBM TE-signature (FIG. 6). L1PA/B/x include L1HS (or L1PA1, among the very few still active TEs in humans) and their closely related subfamilies L1PA(x) and L1PB(x), which are all among the younger subfamilies compared to other LINE-1 subfamilies. In conclusion, certain recent, mainly LINE-1, TE families, preferentially generate HLA-I-presented peptides in GBM.

Because recent TEs have more conserved repeat motifs, it was next sought to investigate if multi-TE encoded HLA-presented peptides corresponded to shared subfamily motifs. The 152 TE subfamilies coding for the 347 identified HLA peptides were represented in 2-dimensional plots coloring the intersections between 2 subfamilies according to the numbers of shared peptides (not shown). The green diagonal in this plot indicates that most subfamilies code for only one peptide. A red square on the diagonal indicates that one TE subfamily can code for more than one peptide. A green square off the diagonal indicates that a peptide can be encoded by TEs from different subfamilies, while a red square outside the diagonal indicates that two different subfamilies code for several shared peptides (up to 25). The class and age of the subfamilies are indicated in color scales on the side of the graph. The three main groups of TE subfamilies coding one or multiple peptides, or redundancy clusters, appear as large squares and are enlarged. The first redundancy cluster (upper left corner) corresponds to a group of L1HS and L1PA(x), two young subfamilies of LINE-1 elements that share up to 25 peptides, pairwise. The second cluster identifies relatively young SINE elements (mainly Alu) that share single peptides (lower right to first group). The third cluster (lower right corner of zoomed panel), corresponds to a group of young subfamilies of SVA elements that share variable numbers of peptides. Therefore, redundancy occurs within multiple TEs from the same recent related subfamilies that could all potentially code for multiple peptides presented on HLA-I molecules. Redundancy in peptide-encoding TEs is therefore limited to a small number of recent TE subfamilies.

To investigate further the links between redundancy and age of TEs, the analysis was extended to all TEs in the genome (redundancy was so far analyzed among GBM TE-signature). Genomic TE-redundancy analysis shows that 49.46% of the 370 peptides identified by immunopeptidomics are encoded by only one TE in the genome (as compared to 85.49% in the scRNAseq GBM TE-signature). At the opposite end, 15.95% of these peptides could potentially be encoded by 201-13500 TE occurrences in the genome. A plot of each peptide according to the number of TEs it can potentially be encoded by, and the age of the corresponding subfamilies was drawn. Among SINEs, Alu-derived peptides are highly redundant and from recent subfamilies, while the MIR-derived peptides are encoded by single TEs from older subfamilies. The same correlation is observed among LINE-1 peptides, with young L1HS, L1PA(x)− and L1PB(x)−derived peptides being encoded by multiple elements, and peptides derived from older L2 and other L 1 subfamilies by unique elements. The negative correlation between the number of TEs potentially encoding single peptides and the age of the corresponding TE subfamilies is confirmed across all TE families (r=−0.61). In conclusion, regardless of TE classes (LINE, SINE, LTR or DNA), subfamilies of young TEs bear shared (redundant) sequences that could code for the same HLA-I peptide, while peptides encoded by TEs from older, more degenerated subfamilies are vastly derived from unique genomic sequences.

### Single-TE Encoded Peptides are More Tumor-Specific

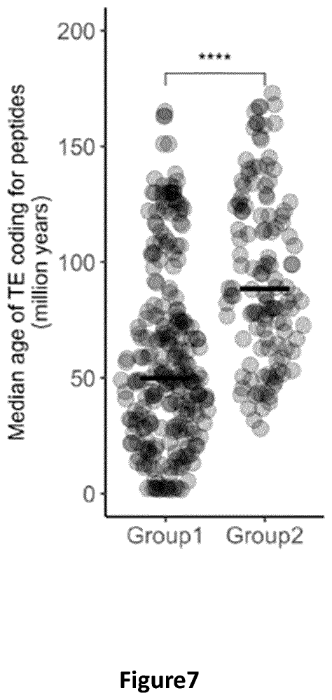

To investigate how redundancy of TE derived peptides affects tumor specificity, the ratio between its expression in TCGA GBM samples and in all healthy tissues from GTEx was represented for each differentially expressed GBM TE from the scRNAseq data set (not shown), brown for higher expression in GBM, blue for the opposite). Unsupervised clustering of the TEs identifies two main groups of peptide coding TEs, group 1 and 2, dominated by TEs overexpressed in GBM and in GTEx, respectively. Group 1 (TEs overexpressed in GTEx) contain higher proportions of LINEs and Others (including all 23 peptide-coding SVA elements), while group 2 contains more LTRs and DNA transposons (FIG. 6, right panels). Moreover, group 1 contains a majority of redundant TEs (63.5%), compared to only 26.6% in group 2 (FIG. 6). Consistently, the median age of group 1 TEs is much lower than the one of Group 2 (FIG. 7). These results show that non-redundant peptides from older TE subfamilies are more likely to be overexpressed in GBM, as compared to healthy tissues than TEs from younger subfamilies encoding redundant peptides.

It was then asked if tumor-specific TEs can be identified. Expression of the top 50 tumor-enriched, peptide-encoding TEs in GBM and all GTEx healthy tissues (as 90 percentile expression, left panel, and percentage of samples with higher expression than GBM median expression, right panel) was determined (not shown). The most tumor-specific TEs are from different classes, but are preferentially derived from ORFs containing a canonical start codon. Some of these TEs are expressed at different levels in a majority of GBM tumors, and undetectable in all, or in a majority, of GTEx healthy tissues (including brain). For some of these TEs, over 90% of the cells expressing the TE are GBM tumor cells in the four patients in the scRNAseq data sets. In conclusion, a subset of unique, non-redundant, peptide-coding TEs are highly tumor-specific and recurrent in cancer patients. These peptide-coding, non-redundant TEs represent interesting potential targets for immunotherapy.

### DISCUSSION

The inventors used here a TE-centered proteogenomic approach to investigate HLA-I presentation of TE-derived peptides, in search for tumor specific recurrent antigens. Two main innovative approaches were combined: i) the pipeline starts with a TE analysis of scRNAseq from total primary tumors, that allows assignment of reads of TEs to GBM cells, and not to hematopoietic or stroma cells, and ii) the alignment for TE mapping was performed to individual TE occurrences, rather than to TE subfamilies, as before (Kong et al., 2019). This sc/individual TE transcriptomic analysis was validated by showing that the differentially expressed TEs were also over expressed in a cohort of 155 bulk RNAseq samples from GBM patients (TCGA), as compared to all tissues, including brain tissue, from healthy donors (GTEx). The signature showed a bias for TEs encoded on chromosome 7, which is frequently amplified in GBM tumor cells, further validating this sc/individual TE strategy. The TE signature was used to interrogate immunopeptidomic mass-spectrometry data bases from 30 GBM primary tumors and cell lines. A set of 347 TE-derived peptides was identified with reliable profiles and motif compliance to HLA alleles of the corresponding samples. These peptides are encoded by 568 TEs, whose analysis revealed some new aspects of the biology of presentation of peptides from TEs in GBM cells. Not all identified peptides, however, are derived from tumor-specific TEs. Further analysis of peptide-coding TEs allowed identification of truly tumor-specific individual TE that actually provide HLA-presented peptides, offering a source of potential targets for immunotherapy.

This study relies largely from scRNAseq mapping of TEs. Several recent papers have analyzed TEs on scRNAseq data sets, and even if a few early studies (He et al., Nat Commun, 2021, 10, 5228. Shao and Wang, Genome Res, 2021, 31, 88-100) pointed to possible bias and limitations, reliable pipelines and guidelines are now available, and have been followed in the present study. These results also rely of different internal controls that support the robustness of these TE scRNAseq analyses. First, it is shown that the TEs expressed in neoplastic GBM cells, but not in other cell populations, are biased for TEs encoded on chromosome 7 (FIG. 1). This corresponds to the known chromosome 7 amplification in GBM and is also detected for coding genes. Second, the GBM TE-signature based on scRNAseq is overexpressed in GBM bulk RNAseq patient cohorts compared to healthy tissues (FIGS. 3C and 3D). Likewise, HLA-I peptidomics with in silico translated RNAseq databases is delicate and can yield numerous false positive identifications. Particular care was taken in validating these TE-derived peptides based on peptide lengths, identification scores, hydrophobicity/RT correlation analyses and binding motif compliance. Furthermore, 23 of the peptides were validated using synthetic peptide comparisons. Importantly, the identified peptides also show the same chromosome 7 bias, which further and independently validates the identifications. One original finding is that the proportions of intronic and intergenic TE occurrences are increased among peptide-coding TEs, as compared to the corresponding proportions in GBM TE-signature (the database used to identify the peptides), at the expense of 3′UTR TEs. HLA-I-presented peptides can therefore be derived from both gene-dependent and gene-independent transcription and translation, but the reasons why intronic TEs provide proportionally more peptides than 3′UTR TEs is worth further analyses. Previous studies found that 3′UTR can code for HLA-presented peptides (Laumont et al., Nat Commun, 2016, 7, 10238; Ruiz Cuevas et al., Cell Rep, 2021, 34, 108815; Zhao et al., Cancer Immunol Res, 2020, 8, 544-555) but these studies did not consider TEs from other genomic locations, as done here. It was also found that LINE-I elements are the major source of HLA-I presented peptides in GBM. LINE-1 represent around 30% of TEs in the human genome, of all TEs expressed in GBM, and of GBM TE-signature, but over 50% of the TE encoded peptides presented on HLA-I. SVA-derived peptides are also strongly enriched, while the proportion of SINE-derived peptides is reduced (as compared to genomic, expressed and differentially expressed SINEs in GBM). LINE-1 elements with and without intact ORFs are preferentially represented among peptide-generating TEs and this bias is observed whether TEs are assigned to multiple or to single locations, indicating that the bias is not due to TE mapping issues.

Another conclusion from this study is that HLA-I molecules present peptides that can be encoded by one or by multiple redundant TEs (bearing the exact same nucleotide sequence encoding the peptide). Other peptides are encoded by TE sequences present only once in the genome. Redundancy, in most cases, occurs within TE subfamilies, and in some cases within different subfamilies that are always from the same TE classes. The most redundant TEs (from several hundred to several thousand occurrences) are from L1PA/B/x and often bear intact annotated ORFs. Peptides derived from Alu (a SINE family member), ERV1 (an LTR family) and SVA (an intermediate length independent family), which are all among the youngest TE families in humans, are also highly represented and redundant. Redundancy is negatively correlated with the age of the TE subfamilies, suggesting that the recurrent sequences encoding HLA-I-binding peptides are part of the ancestral TE insertion event, which subsequently degenerated by mutations and disappeared with time as members of the subfamilies diverged. This scenario is supported by the observation that if 1 or 2 nucleotide mismatches are allowed, the number of redundant TEs is even larger. This is an intriguing observation, and it is not known yet if the peptides identified by mass spectrometry are derived from multiple or unique TE loci. The observation that many of these redundant TEs are actively transcribed and differentially expressed in GBM suggests that the redundant peptides are indeed encoded by multiple loci.

Analysis of the peptide-coding TE ORFs revealed that peptides are generally encoded in 10-100 amino acid long ORFs (with exception of around half of the LINE-encoded peptides that are derived from longer ORFs). In LTRs, peptides are derived from all viral ORFs, with a positive bias for env-derived peptides, as compared to the proportion of env genes annotated in the databases. Among LINE-derived peptides, only a small proportion (around 10%) are derived from the know ORF1p and ORF2p loci. The TE-coding ORFs bear either canonical or alternative start codons, with exception of the longer LINE1 ORFs (over 100 amino acids) which are all driven by canonical ATG start codons.

How, then, it was asked if this knowledge can be used to identify tumor specific TE-derived antigens?Analysis of the relative expression of individual peptide-coding TEs in GBM tumors and a wide series of healthy tissues revealed that redundant TEs from younger subfamilies are generally less tumor-specific than unique TEs from older ones. Because of their more promiscuous expression, it is most likely that the immune system is more tolerized to these antigens from these TEs (although this would need to be addressed specifically). Redundant TEs are therefore probably not the best candidates for tumor-specific targets for immunotherapy, although vaccination with LINE-1 intact ORFs has been shown to be both immunogenic and safe in mice and monkeys (Sacha et al., Immunol, 2012, 189, 1467-1479). These results, however, also identify unique peptide-coding TEs, that are preferentially from MIR, LINE-1 and -2 and some ERV oldest subfamilies. These non-redundant peptide-coding TEs are in majority from relatively old TE subfamilies (over 50 M years), and tBLASTn analysis showed that some of these sequences are present only once in the genome. Some of these TEs are from subfamilies recurrently and selectively de-repressed in tumors, mostly through local DNA demethylation (Brocks et al., Nat Genet, 2017, 49, 1052-1060; Chiappinelli et al., Cell, 2017, 169, 361; Lavie et al., J Virol, 2005, 79, 876-883; Ohtani et al., Cancer Res, 2020, 80, 2441-2450; Roulois et al., Cell, 2015, 1 162, 961-973; Sacha et al., Immunol, 2012, 189, 1467-1479). It is shown that some of these peptide-coding TEs that are expressed in a majority of GBM tumors, are either not detected in healthy tissues or detected at low frequencies and/or low levels.

The results of in vitro stimulation with some of the TE-derived peptides indicate that the TCR repertoire for TEs in healthy individuals exists, opening the possibility that these TEs are immunogenic in patients. Previous studies, however, have shown T cell reactivity against tumor-expressed TEs, establishing the proof of concept that TEs, including ERVs, can be immunogenic in cancer patients (Saini et al., Nat Commun, 2020, 11, 5660; Smith et al.; Wang-Johanning et al., Cancer Res, 2008, 68, 5869-5877). In this context, mapping the expression of individual TEs from single-cell and bulk RNAseq in cancer patients proved efficient in defining individual TE occurrences that yield HLA-I-presented peptides. The tumor-specificity and high recurrence of these peptide-generating TEs opens new perspectives for immunotherapies in many cancer types with de-repressed TEs and beyond, in other immune pathologies where TEs are de-regulated.

### Tables 2 to 4

Table 2 refers to the detailed identification of the TE from neoplastic signature from the present study, corresponding to the transcripts of SEQ ID NO: 381 to 5020. The column numbers refer to the following:
Column n° 1: TE_ID
Column n° 2: Analysis
Column n° 3: Immunopeptidomics_peptide_found
Column n° 4: Peptide_IDs
Column n° 5: Specificity
Column n° 6: TE_Class
Column n° 7: TE_Category
Column n° 8: Genomic_coordinate
Column n° 9: Strand
Column n° 10: Age million
Column n° 11: Length
Column n° 12: Closest gene
Column n° 13: gene_proximity
Column n° 14: Distance_to_closest_gene
Column n° 15: TE_with_gEVE
Column n° 16: TE transcript sequence SEQ ID NO:

According to the conventional nomenclature, TE transcript sequences are disclosed herein as DNA sequences corresponding to the coding DNA

TABLE 2

| 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|
| L1MC4_dup135 | Multi | Yes | PEP: 0001 | Non | LINE | Other L1 | chr1: 10491418-10491630 | − | 120 | 213 | APITD1 | Proximal | 0 | Not documented | 381 |
| | | | | redundant | | | | | | | | | | | |
| L1HS_dup53 | Multi | Yes | PEP: 0324, | Redundant | LINE | L1PA\|B\|x | chr1: 105386456-105392486 | − | 2.7 | 6031 | AMY1C | Distal | 1085145 | EVE annotated | 2 |
| | | | PE | | | | | | | | | | | | |
| SVA_D_dup65 | Multi | Yes | PEP: 0004, | Redundant | Other | SVA | chr1: 109078098-109079480 | − | 25 | 1383 | FAM102B | Distal | 23232 | Not documented | 3 |
| | | | PE | | | | | | | | | | | | |
| L1PA13_dup1 | Uniquely | Yes | PEP: 0111 | Non | LINE | L1PA\|B\|x | chr1: 114030827-114037137 | + | 58 | 6311 | MAGI3 | Proximal | 0 | Not documented | 4 |
| | | | | redundant | | | | | | | | | | | |
| L1MC1_dup576 | Uniquely | Yes | PEP: 0130 | Redundant | LINE | Other L1 | chr1: 119650321-119654546 | − | 69 | 4226 | WARS2 | Proximal | 0 | Not documented | 5 |
| L1PA6_dup3 | Uniquely | Yes | PEP: 0113 | Redundant | LINE | L1PA\|B\|x | chr1: 121241581-121244135 | + | 34 | 2555 | FCGR1B | Distal | 305645 | EVE annotated | 6 |
| L1PA5_dup399 | Multi | Yes | PEP: 0219, | Redundant | LINE | L1PA\|B\|x | chr1: 145047815-145053955 | + | 25 | 6141 | PDE4DIP | Proximal | 0 | EVE annotated | 7 |
| | | | PE | | | | | | | | | | | | |
| L1PA5_dup419 | Multi/ | Yes | PEP: 0053, | Redundant | LINE | L1PA\|B\|x | chr1: 149923965-149930070 | − | 27 | 6106 | OTUD7B | Proximal | 0 | EVE annotated | 8 |
| | Uniquely | | PE | | | | | | | | | | | | |
| LTR5_Hs_dup52 | Multi | Yes | PEP: 0261 | Redundant | LTR | ERVK | chr1: 155596457-155597424 | − | 4.5 | 968 | MSTO1 | Proximal | 0 | EVE annotated | 9 |
| HERVK-int_dup14 | Multi | Yes | PEP: 0175, | Redundant | LTR | ERVK | chr1: 155597425-155599135 | − | 2.3 | 1711 | MSTO1 | Proximal | 0 | EVE annotated | 390 |
| | | | PE | | | | | | | | | | | | |
| LTR5_Hs_dup53 | Multi | Yes | PEP: 0261 | Redundant | LTR | ERVK | chr1: 155604669-155605636 | − | 4.5 | 968 | MSTO1 | Proximal | 0 | Not documented | 1 |
| LTR12F_dup23 | Multi | Yes | PEP: 0048, | Non | LTR | ERV1 | chr1: 155607243-155607452 | − | 86 | 210 | MSTO1 | Proximal | 0 | Not documented | 2 |
| | | | PE | redundant | | | | | | | | | | | |
| L1PA6_dup301 | Uniquely | Yes | PEP: 0303 | Redundant | LINE | L1PA\|B\|x | chr1: 162715794-162721945 | + | 30 | 6152 | DDR2 | Proximal | 0 | Not documented | 3 |
| Kanga2_a_dup79 | Multi | Yes | PEP: 0011 | Non | DNA | DNA | chr1: 166819281-166819572 | + | 150 | 292 | POGK | Proximal | 0 | Not documented | 4 |
| | | | | redundant | | | | | | | | | | | |
| L1P2_dup90 | Uniquely | Yes | PEP: 0303 | Redundant | LINE | L1PA\|B\|x | chr1: 169249076-169253600 | − | 43 | 4525 | NME7 | Proximal | 0 | EVE annotated | 5 |
| AluSz_dup6687 | Multi/ | Yes | PEP: 0338 | Redundant | SINE | Alu | chr1: 202098129-202098436 | − | 49 | 308 | GPR37L1 | Proximal | 0 | Not documented | 6 |
| | Uniquely | | | | | | | | | | | | | | |
| HERVK9-int_dup46 | Uniquely | Yes | PEP: 0195 | Redundant | LTR | ERVK | chr1: 205863345-205864770 | + | 27 | 1426 | SLC26A9 | Distal | 17407 | EVE annotated | 7 |
| L1PA6_dup428 | Multi | Yes | PEP: 0324, | Redundant | LINE | L1PA\|B\|x | chr1: 220778863-220785034 | − | 30 | 6172 | MARK1 | Proximal | 0 | Not documented | 8 |
| | | | PE | | | | | | | | | | | | |
| HAL1_dup1913 | Multi/ | Yes | PEP: 0068 | Non | LINE | Other L1 | chr1: 227490014-227491596 | − | 120 | 1583 | CDC42BPA | Proximal | 0 | Not documented | 9 |
| | Uniquely | | | redundant | | | | | | | | | | | |
| L1PA5_dup718 | Multi | Yes | PEP: 0112, | Redundant | LINE | L1PA\|B\|x | chr1: 242332443-242338355 | + | 41 | 5913 | PLD5 | Proximal | 0 | EVE annotated | 400 |
| | | | PE | | | | | | | | | | | | |
| AluJb_dup4015 | Multi/ | Yes | PEP: 0341, | Redundant | SINE | Alu | chr1: 39575897-39576209 | − | 67 | 313 | MACF1 | Proximal | 0 | Not documented | 1 |
| | Uniquely | | PE | | | | | | | | | | | | |
| AluSc8_dup531 | Multi | Yes | PEP: 0273 | Redundant | SINE | Alu | chr1: 40344660-40344956 | + | 40 | 297 | TRIT1 | Proximal | 0 | Not documented | 2 |
| Charlie2a_dup13 | Multi | Yes | PEP: 0238, | Redundant | DNA | DNA | chr1: 40721880-40722996 | − | 95 | 1117 | ZMPSTE24 | Proximal | 784 | Not documented | 3 |
| | | | PE | | | | | | | | | | | | |
| Charlie2b_dup24 | Multi | Yes | PEP: 0042 | Non | DNA | DNA | chr1: 40723075-40723295 | − | 120 | 221 | ZMPSTE24 | Proximal | 485 | Not documented | 4 |
| | | | | redundant | | | | | | | | | | | |
| L1MA6_dup24 | Multi/ | Yes | PEP: 0141, | Redundant | LINE | Other L1 | chr1: 41633374-41635982 | − | 100 | 2609 | SCMH1 | Proximal | 0 | Not documented | 5 |
| | Uniquely | | PE | | | | | | | | | | | | |
| L2a_dup3767 | Uniquely | Yes | PEP: 0121, | Non | LINE | L2 | chr1: 46535208-46536508 | − | 130 | 1301 | PIK3R3 | Proximal | 0 | Not documented | 6 |
| | | | PE | redundant | | | | | | | | | | | |
| L1PB1_dup157 | Multi/ | Yes | PEP: 0230 | Redundant | LINE | L1PA\|B\|x | chr1: 63205260-63209244 | + | 35 | 3985 | ATG4C | Distal | 40563 | EVE annotated | 7 |
| | Uniquely | | | | | | | | | | | | | | |
| HERV9-int_dup19 | Uniquely | Yes | PEP: 0221 | Redundant | LTR | ERV1 | chr1: 63220509-63225349 | + | 23 | 4841 | ATG4C | Distal | 24458 | EVE annotated | 8 |
| L1HS_dup20 | Multi | Yes | PEP: 0324, | Redundant | LINE | L1PA\|B\|x | chr1: 67544544-67550598 | − | 1.4 | 6055 | C1orf141 | Distal | 7251 | EVE annotated | 9 |
| | | | PE | | | | | | | | | | | | |
| L2a_dup5791 | Uniquely | Yes | PEP: 0070, | Non | LINE | L2 | chr1: 68617114-68618583 | − | 130 | 1470 | WLS | Proximal | 0 | Not documented | 410 |
| | | | PE | redundant | | | | | | | | | | | |
| L2_dup1585 | Uniquely | Yes | PEP: 0248 | Non | LINE | L2 | chr1: 68832447-68832653 | + | 130 | 207 | RPE65 | Distal | 61853 | Not documented | 1 |
| | | | | redundant | | | | | | | | | | | |
| L2b_dup4815 | Multi | Yes | PEP: 0220 | Redundant | LINE | L2 | chr1: 70820751-70820876 | − | 110 | 126 | HHLA3 | Proximal | 0 | Not documented | 2 |
| L1PA16_dup221 | Uniquely | Yes | PEP: 0030 | Non | LINE | L1PA\|B\|x | chr1: 75970521-75972589 | − | 80 | 2069 | SLC44A5 | Proximal | 0 | Not documented | 3 |
| | | | | redundant | | | | | | | | | | | |
| L1HS_dup33 | Multi | Yes | PEP: 0210, | Redundant | LINE | L1PA\|B\|x | chr1: 84518060-84524089 | − | 1.8 | 6030 | PRKACB | Distal | 19657 | EVE annotated | 4 |
| | | | PE | | | | | | | | | | | | |
| L1PA7_dup307 | Multi/ | Yes | PEP: 0324, | Redundant | LINE | L1PA\|B\|x | chr1: 92853960-92860042 | + | 36 | 6083 | RPAP2 | Proximal | 0 | EVE annotated | 5 |
| | Uniquely | | PE | | | | | | | | | | | | |
| MLT1H_dup350 | Multi/ | Yes | PEP: 0123 | Non | LTR | ERVL- | chr1: 93805005-93805156 | + | 110 | 152 | DR1 | Distal | 6290 | Not documented | 6 |
| | Uniquely | | | redundant | | MaLR | | | | | | | | | |
| LTR7B_dup536 | Multi | Yes | PEP: 0268 | Redundant | LTR | ERV1 | chr10: 102140677-102141062 | + | 22 | 386 | SCD | Distal | 16087 | Not documented | 7 |
| L1PA4_dup8539 | Multi | Yes | PEP: 0324, | Redundant | LINE | L1PA\|B\|x | chr10: 102166088-102172241 | + | 22 | 6154 | SCD | Distal | 41498 | EVE annotated | 8 |
| | | | PE | | | | | | | | | | | | |
| L1HS_dup1121 | Multi | Yes | PEP: 0324, | Redundant | LINE | L1PA\|B\|x | chr10: 107137105-107143135 | − | 1.4 | 6031 | SORCS3 | Distal | 112113 | EVE annotated | 9 |
| | | | PE | | | | | | | | | | | | |
| L1PA7_dup9493 | Uniquely | Yes | PEP: 0303 | Redundant | LINE | L1PA\|B\|x | chr10: 120405843-120412254 | − | 37 | 6412 | CACUL1 | Distal | 21426 | EVE annotated | 420 |
| L1MC4a_dup16650 | Uniquely | Yes | PEP: 0198 | Non | LINE | Other L1 | chr10: 25606952-25607631 | − | 110 | 680 | GPR158 | Proximal | 0 | Not documented | 1 |
| | | | | redundant | | | | | | | | | | | |
| L1ME5_dup2184 | Multi | Yes | PEP: 0328 | Non | LINE | Other L1 | chr10: 31616339-31616948 | − | 130 | 610 | ZEB1 | Proximal | 0 | Not documented | 2 |
| | | | | redundant | | | | | | | | | | | |
| L1PB3_dup2485 | Uniquely | Yes | PEP: 0054, | Non | LINE | L1PA\|B\|x | chr10: 60294142-60299303 | + | 75 | 5162 | BICC1 | Proximal | 0 | EVE annotated | 3 |
| | | | PE | redundant | | | | | | | | | | | |
| MamRep605_dup3009 | Multi/ | Yes | PEP: 0103 | Redundant | Other | Other | chr10: 60934720-60934951 | + | 160 | 232 | PHYHIPL | Proximal | 1400 | Not documented | 4 |
| | Uniquely | | | | | Repeats | | | | | | | | | |
| L1HS_dup1087 | Multi | Yes | PEP: 0324, | Redundant | LINE | L1PA\|B\|x | chr10: 6411598-6417629 | − | 1.8 | 6032 | DKFZP667F0711 | Distal | 16876 | EVE annotated | 5 |
| | | | PE | | | | | | | | | | | | |
| SVA_D_dup817 | Multi | Yes | PEP: 0075, | Redundant | Other | SVA | chr10: 73628416-73630264 | − | 16 | 1849 | PSAP | Distal | 17291 | Not documented | 6 |
| | | | PE | | | | | | | | | | | | |
| L2a_dup107251 | Uniquely | Yes | PEP: 0254 | Redundant | LINE | L2 | chr10: 83813380-83814521 | − | 140 | 1142 | NRG3 | Proximal | 0 | Not documented | 7 |
| L1PA6_dup4237 | Uniquely | Yes | PEP: 0113, | Redundant | LINE | L1PA\|B\|x | chr10: 96865056-96871181 | − | 35 | 6126 | CYP2C8 | Distal | 35803 | EVE annotated | 8 |
| | | | PE | | | | | | | | | | | | |
| HERVK-int_dup176 | Multi | Yes | PEP: 0200 | Redundant | LTR | ERVK | chr11: 101566762-101574291 | + | 2.7 | 7530 | TRPC6 | Proximal | 0 | EVE annotated | 9 |
| L1ME4a_dup30100 | Multi | Yes | PEP: 0138 | Non | LINE | Other L1 | chr11: 105968605-105969023 | − | 130 | 419 | AASDHPPT | Proximal | 0 | Not documented | 430 |
| | | | | redundant | | | | | | | | | | | |
| Charlie1_dup1272 | Multi/ | Yes | PEP: 0320, | Non | DNA | DNA | chr11: 10874252-10876178 | − | 130 | 1927 | ZBED5 | Proximal | 0 | Not documented | 1 |
| | Uniquely | | PE | redundant | | | | | | | | | | | |
| L1PA5_dup8498 | Multi | Yes | PEP: 0324, | Redundant | LINE | L1PA\|B\|x | chr11: 113094670-113100816 | + | 25 | 6147 | NCAM1 | Proximal | 0 | EVE annotated | 2 |
| | | | PE | | | | | | | | | | | | |
| L1HS_dup1201 | Multi | Yes | PEP: 0324, | Redundant | LINE | L1PA\|B\|x | chr11: 125406506-125412536 | + | 2.7 | 6031 | EI24 | Distal | 26577 | EVE annotated | 3 |
| | | | PE | | | | | | | | | | | | |
| L1MB8_dup10879 | Uniquely | Yes | PEP: 0118 | Non | LINE | Other L1 | chr11: 12802088-12803387 | + | 87 | 1300 | TEAD1 | Proximal | 0 | Not documented | 4 |
| | | | | redundant | | | | | | | | | | | |
| MIRc_dup67159 | Multi | Yes | PEP: 0302 | Non | SINE | MIR | chr11: 17304345-17304486 | + | 120 | 142 | NUCB2 | Proximal | 0 | Not documented | 5 |
| | | | | redundant | | | | | | | | | | | |
| SVA_D_dup851 | Multi | Yes | PEP: 0075, | Redundant | Other | SVA | chr11: 19880686-19882618 | − | 11 | 1933 | NAV2 | Proximal | 0 | Not documented | 6 |
| | | | PE | | | | | | | | | | | | |
| L1PA7_dup9595 | Multi/ | Yes | PEP: 0145, | Redundant | LINE | L1PA\|B\|x | chr11: 20092505-20096486 | − | 38 | 3982 | NAV2 | Proximal | 0 | EVE annotated | 7 |
| | Uniquely | | PE | | | | | | | | | | | | |
| L1HS_dup1149 | Multi | Yes | PEP: 0324, | Redundant | LINE | L1PA\|B\|x | chr11: 24349498-24355547 | + | 1.8 | 6050 | LUZP2 | Distal | 162970 | EVE annotated | 8 |
| | | | PE | | | | | | | | | | | | |
| L1PA5_dup8001 | Uniquely | Yes | PEP: 0303 | Redundant | LINE | L1PA\|B\|x | chr11: 2655405-2661585 | − | 27 | 6181 | KCNQ1 | Proximal | 0 | EVE annotated | 9 |
| L1PA5_dup8143 | Uniquely | Yes | PEP: 0303 | Redundant | LINE | L1PA\|B\|x | chr11: 27686849-27692569 | + | 32 | 5721 | BDNF | Proximal | 0 | Not documented | 440 |
| LTR25-int_dup441 | Uniquely | Yes | PEP: 0189 | Redundant | LTR | ERV1 | chr11: 28158080-28161203 | − | 75 | 3124 | METTL15 | Proximal | 0 | Not documented | 1 |
| Tigger6a_dup561 | Multi | Yes | PEP: 0315 | Non | DNA | DNA | chr11: 30358575-30359619 | + | 89 | 1045 | ARL14EP | Proximal | 0 | Not documented | 2 |
| | | | | redundant | | | | | | | | | | | |
| L1PB4_dup5262 | Multi/ | Yes | PEP: 0204 | Redundant | LINE | L1PA\|B\|x | chr11: 33911212-33911757 | + | 57 | 546 | LMO2 | Proximal | 0 | Not documented | 3 |
| | Uniquely | | | | | | | | | | | | | | |
| L1PA4_dup8796 | Multi/ | Yes | PEP: 0053, | Redundant | LINE | L1PA\|B\|x | chr11: 35776053-35782191 | − | 23 | 6139 | TRIM44 | Proximal | 0 | EVE annotated | 4 |
| | Uniquely | | PE | | | | | | | | | | | | |
| L1PA5_dup8179 | Multi/ | Yes | PEP: 0053, | Redundant | LINE | L1PA\|B\|x | chr11: 35795022-35801113 | − | 30 | 6092 | TRIM44 | Proximal | 0 | EVE annotated | 5 |
| | Uniquely | | PE | | | | | | | | | | | | |
| L1HS_dup1157 | Multi | Yes | PEP: 0324, | Redundant | LINE | L1PA\|B\|x | chr11: 36573157-36579186 | − | 1.4 | 6030 | RAG1 | Proximal | 0 | EVE annotated | 6 |
| | | | PE | | | | | | | | | | | | |
| L1M4c_dup4425 | Multi | Yes | PEP: 0144 | Non | LINE | Other L1 | chr11: 65267057-65267487 | + | 140 | 431 | SCYL1 | Distal | 25062 | Not documented | 7 |
| | | | | redundant | | | | | | | | | | | |
| HAL1_dup18654 | Multi | Yes | PEP: 0361 | Non | LINE | Other L1 | chr11: 66300846-66301152 | − | 100 | 307 | ZDHHC24 | Proximal | 0 | Not documented | 8 |
| | | | | redundant | | | | | | | | | | | |
| MER49_dup896 | Multi | Yes | PEP: 0023, | Non | LTR | ERV1 | chr11: 71162311-71163232 | + | 67 | 922 | DHCR7 | Proximal | 0 | Not documented | 9 |
| | | | PE | redundant | | | | | | | | | | | |
| L1M4_dup11911 | Uniquely | Yes | PEP: 0108 | Redundant | LINE | Other L1 | chr11: 74541078-74543102 | + | 81 | 2025 | RNF169 | Proximal | 0 | Not documented | 450 |
| L1HS_dup1176 | Multi | Yes | PEP: 0324, | Redundant | LINE | L1PA\|B\|x | chr11: 78388818-78394847 | − | 1.4 | 6030 | TENM4 | Proximal | 0 | EVE annotated | 1 |
| | | | PE | | | | | | | | | | | | |
| AluSz_dup57983 | Multi | Yes | PEP: 0077 | Redundant | SINE | Alu | chr11: 784155-784462 | + | 44 | 308 | AP006621.5 | Proximal | 0 | Not documented | 2 |
| L1PA4_dup9057 | Uniquely | Yes | PEP: 0113, | Redundant | LINE | L1PA\|B\|x | chr11: 81245341-81251463 | − | 21 | 6123 | FAM181B | Distal | 1191591 | EVE annotated | 3 |
| | | | PE | | | | | | | | | | | | |
| L1HS_dup1178 | Multi | Yes | PEP: 0324, | Redundant | LINE | L1PA\|B\|x | chr11: 81866908-81872933 | + | 2.7 | 6026 | FAM181B | Distal | 570121 | EVE annotated | 4 |
| | | | PE | | | | | | | | | | | | |
| L1PA15_dup6234 | Uniquely | Yes | PEP: 0013 | Non | LINE | L1PA\|B\|x | chr11: 85165222-85171708 | − | 76 | 6487 | DLG2 | Proximal | 0 | Not documented | 5 |
| | | | | redundant | | | | | | | | | | | |
| L1HS_dup1185 | Multi | Yes | PEP: 0324, | Redundant | LINE | L1PA\|B\|x | chr11: 92869805-92875839 | + | 1.4 | 6035 | SLC36A4 | Proximal | 1503 | EVE annotated | 6 |
| | | | PE | | | | | | | | | | | | |
| L1PA3_dup8142 | Multi | Yes | PEP: 0210, | Redundant | LINE | L1PA\|B\|x | chr11: 93149299-93152361 | + | 13 | 3063 | CCDC67 | Proximal | 0 | EVE annotated | 7 |
| | | | PE | | | | | | | | | | | | |
| L1HS_dup1186 | Multi | Yes | PEP: 0324, | Redundant | LINE | L1PA\|B\|x | chr11: 93154153-93160197 | + | 1.4 | 6045 | CCDC67 | Proximal | 0 | EVE annotated | 8 |
| | | | PE | | | | | | | | | | | | |
| L1M5_dup44273 | Multi | Yes | PEP: 0091 | Non | LINE | Other L1 | chr11: 94802434-94802578 | − | 140 | 145 | ENDOD1 | Distal | 20397 | Not documented | 9 |
| | | | | redundant | | | | | | | | | | | |
| L1HS_dup1188 | Multi | Yes | PEP: 0210, | Redundant | LINE | L1PA\|B\|x | chr11: 95169381-95175410 | + | 1.4 | 6030 | SESN3 | Distal | 203677 | EVE annotated | 460 |
| | | | PE | | | | | | | | | | | | |
| SVA_F_dup721 | Multi/ | Yes | PEP: 0012, | Redundant | Other | SVA | chr12: 104716578-104717542 | − | 21 | 965 | TXNRD1 | Proximal | 0 | Not documented | 1 |
| | Uniquely | | PE | | | | | | | | | | | | |
| L1PA8_dup6016 | Multi/ | Yes | PEP: 0053, | Redundant | LINE | L1PA\|B\|x | chr12: 107475814-107479613 | + | 34 | 3800 | CRY1 | Proximal | 0 | EVE annotated | 2 |
| | Uniquely | | PE | | | | | | | | | | | | |
| ERVL-E-int_dup6539 | Uniquely | Yes | PEP: 0181 | Non | LTR | ERVL | chr12: 11134596-11135836 | + | 160 | 1241 | TAS2R14 | Proximal | 0 | Not documented | 3 |
| | | | | redundant | | | | | | | | | | | |
| L1HS_dup1256 | Multi | Yes | PEP: 0210, | Redundant | LINE | L1PA\|B\|x | chr12: 126783557-126789584 | − | 1.4 | 6028 | TMEM132B | Distal | 636641 | EVE annotated | 4 |
| | | | PE | | | | | | | | | | | | |
| SVA_D_dup975 | Multi | Yes | PEP: 0284, | Redundant | Other | SVA | chr12: 132410290-132411662 | − | 19 | 1373 | PUS1 | Distal | 2084 | Not documented | 5 |
| | | | PE | | | | | | | | | | | | |
| L1PA3_dup8332 | Multi | Yes | PEP: 0324, | Redundant | LINE | L1PA\|B\|x | chr12: 20820770-20826793 | + | 12 | 6024 | PDE3A | Proximal | 0 | EVE annotated | 6 |
| | | | PE | | | | | | | | | | | | |
| L1PA4_dup9392 | Uniquely | Yes | PEP: 0113, | Redundant | LINE | L1PA\|B\|x | chr12: 31703001-31709139 | − | 18 | 6139 | DENND5B | Proximal | 0 | EVE annotated | 7 |
| | | | PE | | | | | | | | | | | | |
| L1PA3_dup8450 | Uniquely | Yes | PEP: 0113, | Redundant | LINE | L1PA\|B\|x | chr12: 45764630-45770648 | + | 12 | 6019 | ANO6 | Proximal | 0 | EVE annotated | 8 |
| | | | PE | | | | | | | | | | | | |
| SVA_D_dup928 | Multi | Yes | PEP: 0284, | Redundant | Other | SVA | chr12: 49235222-49237100 | + | 18 | 1879 | DDX23 | Proximal | 0 | Not documented | 9 |
| | | | PE | | | | | | | | | | | | |
| SVA_D_dup931 | Multi | Yes | PEP: 0075, | Redundant | Other | SVA | chr12: 49829429-49830392 | − | 31 | 964 | SPATS2 | Proximal | 0 | Not documented | 470 |
| | | | PE | | | | | | | | | | | | |
| L1PA7_dup10346 | Multi/ | Yes | PEP: 0015, | Redundant | LINE | L1PA\|B\|x | chr12: 58820015-58824933 | + | 41 | 4919 | RP11-362K2.2 | Distal | 112975 | EVE annotated | 1 |
| | Uniquely | | PE | | | | | | | | | | | | |
| L1HS_dup1233 | Multi | Yes | PEP: 0324, | Redundant | LINE | L1PA\|B\|x | chr12: 70167191-70173221 | − | 2.3 | 6031 | RAB3IP | Proximal | 0 | EVE annotated | 2 |
| | | | PE | | | | | | | | | | | | |
| MLT2B2_dup1668 | Multi | Yes | PEP: 0193 | Non | LTR | ERVL | chr12: 70573879-70574408 | + | 93 | 530 | CNOT2 | Distal | 62367 | Not documented | |
| | | | | redundant | | | | | | | | | | | |
| SVA_E_dup173 | Multi | Yes | PEP: 0075, | Redundant | Other | SVA | chr12: 70596315-70597562 | + | 32 | 1248 | CNOT2 | Distal | 39213 | Not documented | 4 |
| | | | PE | | | | | | | | | | | | |
| AluY_dup80635 | Multi | Yes | PEP: 0338 | Redundant | SINE | Alu | chr12: 70656048-70656354 | − | 27 | 307 | CNOT2 | Proximal | 0 | Not documented | 5 |
| AluSz_dup62220 | Multi | Yes | PEP: 0341 | Redundant | SINE | Alu | chr12: 7085605-7085893 | + | 44 | 289 | EMG1 | Proximal | 0 | Not documented | 6 |
| HERVL-int_dup1822 | Uniquely | Yes | PEP: 0249 | Redundant | LTR | ERVL | chr12: 75766093-75771061 | − | 49 | 4969 | CAPS2 | Proximal | 0 | EVE annotated | 7 |
| L1M4c_dup4562 | Uniquely | Yes | PEP: 0149 | Redundant | LINE | Other L1 | chr12: 9841361-9844457 | + | 110 | 3097 | CLEC2D | Proximal | 0 | Not documented | 8 |
| L1P3_dup2710 | Uniquely | Yes | PEP: 0303 | Redundant | LINE | L1PA\|B\|x | chr13: 24167940-24172311 | + | 40 | 4372 | TNFRSF19 | Proximal | 0 | Not documented | 9 |
| L1HS_dup1258 | Multi | Yes | PEP: 0210, | Redundant | LINE | L1PA\|B\|x | chr13: 30215844-30221843 | − | 1.4 | 6000 | SLC7A1 | Distal | 46020 | EVE annotated | 480 |
| | | | PE | | | | | | | | | | | | |
| MER21B_dup2011 | Uniquely | Yes | PEP: 0143 | Non | LTR | ERVL | chr13: 31871592-31872407 | − | 77 | 816 | B3GALTL | Proximal | 0 | Not documented | 1 |
| | | | | redundant | | | | | | | | | | | |
| L1HS_dup1261 | Multi | Yes | PEP: 0324, | Redundant | LINE | L1PA\|B\|x | chr13: 35054400-35060431 | − | 2.7 | 6032 | NBEA | Distal | 455994 | EVE annotated | 2 |
| | | | PE | | | | | | | | | | | | |
| L1MC2_dup4820 | Uniquely | Yes | PEP: 0064, | Non | LINE | Other L1 | chr13: 45101485-45105120 | + | 75 | 3636 | TSC22D1 | Proximal | 0 | Not documented | 3 |
| | | | PE | redundant | | | | | | | | | | | |
| ALR/Alpha_dup1020 | Multi | Yes | PEP: 0360, | Redundant | Other | Other | chr14: 19000006-19048872 | − | 63 | 48867 | OR11H12 | Distal | 328651 | Not documented | 4 |
| | | | PE | | | Repeats | | | | | | | | | |
| CER_dup30 | Multi/ | Yes | PEP: 0216, | Redundant | Other | Other | chr14: 19215371-19320499 | − | 67 | 105129 | OR11H12 | Distal | 57024 | Not documented | 5 |
| | Uniquely | | PE | | | Repeats | | | | | | | | | |
| L1PA3_dup9152 | Multi | Yes | PEP: 0231, | Redundant | LINE | L1PA\|B\|x | chr14: 28882082-28883407 | − | 11 | 1326 | FOXG1 | Distal | 351644 | Not documented | 6 |
| | | | PE | | | | | | | | | | | | |
| L1HS_dup1306 | Multi | Yes | PEP: 0210, | Redundant | LINE | L1PA\|B\|x | chr14: 32263063-32267338 | − | 3.2 | 4276 | NUBPL | Proximal | 0 | EVE annotated | 7 |
| | | | PE | | | | | | | | | | | | |
| L1PA5_dup9524 | Multi/ | Yes | PEP: 0324, | Redundant | LINE | L1PA\|B\|x | chr14: 33557268-33563332 | + | 32 | 6065 | NPAS3 | Proximal | 0 | EVE annotated | 8 |
| | Uniquely | | PE | | | | | | | | | | | | |
| MLT1G1_dup2780 | Uniquely | Yes | PEP: 0368 | Redundant | LTR | ERVL- | chr14: 33939014-33939565 | − | 120 | 552 | NPAS3 | Proximal | 0 | Not documented | 9 |
| | | | | | | MaLR | | | | | | | | | |
| L1HS_dup1314 | Multi | Yes | PEP: 0324, | Redundant | LINE | L1PA\|B\|x | chr14: 45946341-45952372 | − | 3.6 | 6032 | MIS18BP1 | Distal | 223599 | EVE annotated | 490 |
| | | | PE | | | | | | | | | | | | |
| MIR_dup136799 | Uniquely | Yes | PEP: 0072 | Non | SINE | MIR | chr14: 51543262-51543524 | − | 120 | 263 | TRIM9 | Proximal | 0 | Not documented | 1 |
| | | | | redundant | | | | | | | | | | | |
| AluSx1_dup77995 | Multi/ | Yes | PEP: 0338 | Redundant | SINE | Alu | chr14: 55185440-55185745 | + | 50 | 306 | SAMD4A | Proximal | 0 | Not documented | 2 |
| | Uniquely | | | | | | | | | | | | | | |
| AluSg_dup29867 | Multi | Yes | PEP: 0201 | Redundant | SINE | Alu | chr14: 55534120-55534420 | − | 47 | 301 | MAPK1IP1L | Proximal | 0 | Not documented | 3 |
| L1HS_dup1332 | Multi | Yes | PEP: 0324, | Redundant | LINE | L1PA\|B\|x | chr14: 71014008-71020039 | + | 2.3 | 6032 | ADAM20 | Distal | 12277 | EVE annotated | 4 |
| | | | PE | | | | | | | | | | | | |
| AluSp_dup35870 | Multi | Yes | PEP: 0338 | Redundant | SINE | Alu | chr14: 74525506-74525809 | + | 35 | 304 | ALDH6A1 | Proximal | 0 | Not documented | 5 |
| SVA_A_dup203 | Multi | Yes | PEP: 0200 | Redundant | Other | SVA | chr14: 92653391-92654300 | − | 32 | 910 | CPSF2 | Distal | 22637 | Not documented | 6 |
| ALR/Alpha_dup1025 | Multi | Yes | PEP: 0336 | Redundant | Other | Other | chr15: 20000001-20022125 | − | 71 | 22125 | GOLGA6L6 | Distal | 714970 | Not documented | 7 |
| | | | | | | Repeats | | | | | | | | | |
| L1HS_dup1341 | Multi | Yes | PEP: 0231 | Redundant | LINE | L1PA\|B\|x | chr15: 20022226-20023450 | − | 0.91 | 1225 | GOLGA6L6 | Distal | 713645 | Not documented | 8 |
| L1PA5_dup9834 | Multi | Yes | PEP: 0219, | Redundant | LINE | L1PA\|B\|x | chr15: 25500761-25506894 | + | 30 | 6134 | UBE3A | Distal | 75488 | EVE annotated | 9 |
| | | | PE | | | | | | | | | | | | |
| SVA_D_dup1058 | Multi | Yes | PEP: 0075, | Redundant | Other | SVA | chr15: 27248371-27249814 | − | 11 | 1444 | GABRG3 | Proximal | 0 | Not documented | 500 |
| | | | PE | | | | | | | | | | | | |
| L1MC1_dup11204 | Uniquely | Yes | PEP: 0106 | Non | LINE | Other L1 | chr15: 35661914-35663738 | + | 74 | 1825 | DPH6 | Proximal | 0 | Not documented | 1 |
| | | | | redundant | | | | | | | | | | | |
| AluSx1_dup80906 | Multi | Yes | PEP: 0338 | Redundant | SINE | Alu | chr15: 41590812-41591106 | + | 51 | 295 | OIP5 | Distal | 10361 | Not documented | 2 |
| L1PA5_dup9899 | Uniquely | Yes | PEP: 0303 | Redundant | LINE | L1PA\|B\|x | chr15: 46717128-46723258 | + | 33 | 6131 | FKSG62 | Distal | 702436 | Not documented | 3 |
| L1M1_dup8362 | Uniquely | Yes | PEP: 0334 | Non | LINE | Other L1 | chr15: 46809737-46813656 | + | 99 | 3920 | FKSG62 | Distal | 612038 | Not documented | 4 |
| | | | | redundant | | | | | | | | | | | |
| SVA_D_dup1070 | Multi | Yes | PEP: 0284, | Redundant | Other | SVA | chr15: 50449925-50451308 | + | 20 | 1384 | ATP8B4 | Proximal | 0 | Not documented | 5 |
| | | | PE | | | | | | | | | | | | |
| SVA_D_dup1072 | Multi | Yes | PEP: 0075, | Redundant | Other | SVA | chr15: 52577645-52579153 | + | 20 | 1509 | MYO5C | Proximal | 0 | Not documented | 6 |
| | | | PE | | | | | | | | | | | | |
| SVA_D_dup1074 | Multi | Yes | PEP: 0075, | Redundant | Other | SVA | chr15: 56402252-56403872 | + | 34 | 1621 | RFX7 | Proximal | 0 | Not documented | 7 |
| | | | PE | | | | | | | | | | | | |
| L1PA6_dup5230 | Uniquely | Yes | PEP: 0113 | Redundant | LINE | L1PA\|B\|x | chr15: 56691188-56697340 | + | 31 | 6153 | TEX9 | Proximal | 0 | EVE annotated | 8 |
| SVA_C_dup230 | Multi | Yes | PEP: 0284, | Redundant | Other | SVA | chr15: 61238433-61239907 | + | 14 | 1475 | RORA | Proximal | 0 | Not documented | 9 |
| | | | PE | | | | | | | | | | | | |
| L1HS_dup1360 | Multi | Yes | PEP: 0324, | Redundant | LINE | L1PA\|B\|x | chr15: 71022084-71027499 | + | 0.91 | 5416 | UACA | Proximal | 0 | EVE annotated | 510 |
| | | | PE | | | | | | | | | | | | |
| L1PA7_dup11763 | Uniquely | Yes | PEP: 0290, | Redundant | LINE | L1PA\|B\|x | chr15: 71260341-71266080 | − | 39 | 5740 | LRRC49 | Proximal | 0 | Not documented | 1 |
| | | | PE | | | | | | | | | | | | |
| AluSx1_dup82767 | Multi | Yes | PEP: 0077 | Redundant | SINE | Alu | chr15: 80199851-80200079 | + | 55 | 229 | ST20 | Proximal | 0 | Not documented | 2 |
| L1HS_dup1366 | Multi | Yes | PEP: 0210, | Redundant | LINE | L1PA\|B\|x | chr15: 83551646-83557671 | − | 1.4 | 6026 | HOMER2 | Proximal | 0 | EVE annotated | 3 |
| | | | PE | | | | | | | | | | | | |
| MIR3_dup75620 | Multi/ | Yes | PEP: 0133 | Non | SINE | MIR | chr15: 83673660-83673823 | + | 160 | 164 | C15orf40 | Proximal | 0 | Not documented | 4 |
| | Uniquely | | | redundant | | | | | | | | | | | |
| HERV4_I-int_dup288 | Uniquely | Yes | PEP: 0335, | Non | LTR | ERV1 | chr15: 90006206-90012622 | + | 37 | 6417 | RHCG | Proximal | 0 | EVE annotated | 5 |
| | | | PE | redundant | | | | | | | | | | | |
| L1HS_dup1374 | Multi | Yes | PEP: 0324, | Redundant | LINE | L1PA\|B\|x | chr16: 16934394-16940413 | − | 1.8 | 6020 | XYLT1 | Distal | 255214 | EVE annotated | 6 |
| | | | PE | | | | | | | | | | | | |
| ALR/Alpha_dup1050 | Multi | Yes | PEP: 0087 | Redundant | Other | Other | chr16: 33902322-33910905 | − | 71 | 8584 | RP11-812E19.9 | Distal | 254627 | Not documented | 7 |
| | | | | | | Repeats | | | | | | | | | |
| ALR/Alpha_dup1055 | Multi | Yes | PEP: 0087 | Redundant | Other | Other | chr16: 33981456-33997390 | + | 72 | 15935 | CTD-2144E22.5 | Distal | 259118 | Not documented | 8 |
| | | | | | | Repeats | | | | | | | | | |
| ALR/Alpha_dup1060 | Multi | Yes | PEP: 0087, | Redundant | Other | Other | chr16: 34005354-34016791 | − | 70 | 11438 | CTD-2144E22.5 | Distal | 239717 | EVE annotated | 9 |
| | | | PE | | | Repeats | | | | | | | | | |
| L1HS_dup1382 | Multi | Yes | PEP: 0324, | Redundant | LINE | L1PA\|B\|x | chr16: 34016792-34021340 | − | 17 | 4549 | CTD-2144E22.5 | Distal | 235168 | EVE annotated | 520 |
| | | | PE | | | | | | | | | | | | |
| MIR3_dup77616 | Multi | Yes | PEP: 0343 | Non | SINE | MIR | chr16: 56717268-56717422 | − | 140 | 155 | MT1X | Proximal | 0 | Not documented | 1 |
| | | | | redundant | | | | | | | | | | | |
| L1HS_dup1395 | Multi | Yes | PEP: 0210, | Redundant | LINE | L1PA\|B\|x | chr16: 65723915-65729923 | + | 5 | 6009 | CDH11 | Distal | 563901 | EVE annotated | 2 |
| | | | PE | | | | | | | | | | | | |
| AluSz6_dup37377 | Multi/ | Yes | PEP: 0074 | Redundant | SINE | Alu | chr16: 70702034-70702322 | + | 71 | 289 | MTSS1L | Proximal | 0 | Not documented | 3 |
| | Uniquely | | | | | | | | | | | | | | |
| AluSq2_dup44494 | Multi/ | Yes | PEP: 0339 | Redundant | SINE | Alu | chr16: 70705970-70706273 | + | 50 | 304 | MTSS1L | Proximal | 0 | Not documented | 4 |
| | Uniquely | | | | | | | | | | | | | | |
| L1MA2_dup6686 | Uniquely | Yes | PEP: 0332, | Redundant | LINE | Other L1 | chr16: 72482343-72487601 | − | 53 | 5259 | PMFBP1 | Distal | 271567 | Not documented | 5 |
| | | | PE | | | | | | | | | | | | |
| L1HS_dup1372 | Multi | Yes | PEP: 0210, | Redundant | LINE | L1PA\|B\|x | chr16: 8107922-8110803 | + | 2.3 | 2882 | RBFOX1 | Distal | 344583 | EVE annotated | 6 |
| | | | PE | | | | | | | | | | | | |
| L1HS_dup1399 | Multi | Yes | PEP: 0324, | Redundant | LINE | L1PA\|B\|x | chr16: 83670858-83676901 | + | 1.4 | 6044 | CDH13 | Proximal | | EVE annotated | 7 |
| | | | PE | | | | | | | | | | | | |
| L1MDa_dup6175 | Uniquely | Yes | PEP: 0096 | Redundant | LINE | Other L1 | chr17: 19938218-19938923 | − | 83 | 706 | SPECC1 | Proximal | 0 | Not documented | 8 |
| HERVK9-int_dup571 | Uniquely | Yes | PEP: 0107 | Non | LTR | ERVK | chr17: 20981128-20983385 | − | 51 | 2258 | USP22 | Distal | 34056 | EVE annotated | 9 |
| | | | | redundant | | | | | | | | | | | |
| AluSz6_dup38489 | Multi | Yes | PEP: 0008 | Redundant | SINE | Alu | chr17: 33477943-33478114 | − | 63 | 172 | UNC45B | Proximal | 0 | Not documented | 530 |
| Harlequin-int_dup432 | Uniquely | Yes | PEP: 0038 | Non | LTR | ERV1 | chr17: 41312738-41317903 | − | 27 | 5166 | NBR1 | Distal | 4596 | EVE annotated | 1 |
| | | | | redundant | | | | | | | | | | | |
| Tigger5_dup2360 | Uniquely | Yes | PEP: 0298, | Non | DNA | DNA | chr17: 57776702-57777518 | − | 48 | 817 | PTRH2 | Proximal | 0 | Not documented | 2 |
| | | | PE | redundant | | | | | | | | | | | |
| MIR3_dup78920 | Multi | Yes | PEP: 0301 | Non | SINE | MIR | chr17: 6027127-6027256 | + | 120 | 130 | WSCD1 | Proximal | 0 | Not documented | 3 |
| | | | | redundant | | | | | | | | | | | |
| AluJb_dup123717 | Uniquely | Yes | PEP: 0089 | Non | SINE | Alu | chr17: 66102544-66102866 | − | 75 | 323 | KPNA2 | Distal | 59587 | Not documented | 4 |
| | | | | redundant | | | | | | | | | | | |
| L1HS_dup1413 | Multi | Yes | PEP: 0210, | Redundant | LINE | L1PA\|B\|x | chr17: 68455098-68461128 | + | 1.4 | 6031 | KCNJ2 | Distal | 278910 | EVE annotated | 5 |
| | | | PE | | | | | | | | | | | | |
| AluSx_dup121658 | Multi | Yes | PEP: 0008, | Redundant | SINE | Alu | chr17: 74560233-74560505 | − | 60 | 273 | ST6GALNAC2 | Proximal | 0 | Not documented | 6 |
| | | | PE | | | | | | | | | | | | |
| THE1B_dup20543 | Multi | Yes | PEP: 0251 | Non | LTR | ERVL- | chr17: 74560657-74560763 | + | 51 | 107 | ST6GALNAC2 | Proximal | 0 | Not documented | 7 |
| | | | | redundant | | MaLR | | | | | | | | | |
| MER76_dup633 | Multi | Yes | PEP: 0245 | Non | LTR | ERVL | chr17: 80005780-80006205 | − | 120 | 426 | RFNG | Proximal | 0 | Not documented | 8 |
| | | | | redundant | | | | | | | | | | | |
| CER_dup34 | Multi | Yes | PEP: 0356 | Non | Other | Other | chr18: 15235272-15246086 | + | 59 | 10815 | ANKRD30B | Distal | 382536 | Not documented | 9 |
| | | | | redundant | | Repeats | | | | | | | | | |
| AluJr_dup67276 | Multi/ | Yes | PEP: 0270 | Redundant | SINE | Alu | chr18: 22032766-22033030 | − | 75 | 265 | IMPACT | Proximal | 0 | Not documented | 540 |
| | Uniquely | | | | | | | | | | | | | | |
| L1MEf_dup12734 | Uniquely | Yes | PEP: 0149 | Redundant | LINE | Other L1 | chr18: 31786186-31788259 | − | 130 | 2074 | NOL4 | Proximal | 0 | Not documented | 1 |
| L2c_dup130480 | Uniquely | Yes | PEP: 0063, | Non | LINE | L2 | chr18: 32198764-32200368 | − | 160 | 1605 | DTNA | Proximal | 0 | Not documented | 2 |
| | | | PE | redundant | | | | | | | | | | | |
| L2b_dup87581 | Uniquely | Yes | PEP: 0078 | Non | LINE | L2 | chr18: 32280212-32282205 | + | 150 | 1994 | DTNA | Proximal | 0 | Not documented | 3 |
| | | | | redundant | | | | | | | | | | | |
| AluJo_dup62764 | Multi | Yes | PEP: 0270 | Redundant | SINE | Alu | chr18: 32404712-32404809 | − | 79 | 98 | DTNA | Proximal | 0 | Not documented | 4 |
| L1PA3_dup10033 | Multi | Yes | PEP: 0324, | Redundant | LINE | L1PA\|B\|x | chr18: 32474739-32480892 | + | 17 | 6154 | DTNA | Distal | 2932 | EVE annotated | 5 |
| | | | PE | | | | | | | | | | | | |
| L1PA10_dup6514 | Multi/ | Yes | PEP: 0003, | Redundant | LINE | L1PA\|B\|x | chr18: 32839824-32841822 | − | 34 | 1999 | ZNF397 | Proximal | 0 | Not documented | 6 |
| | Uniquely | | PE | | | | | | | | | | | | |
| L1HS_dup1439 | Multi | Yes | PEP: 0169, | Redundant | LINE | L1PA\|B\|x | chr18: 41031549-41032491 | + | 2.3 | 943 | SYT4 | Distal | 173935 | Not documented | 7 |
| | | | PE | | | | | | | | | | | | |
| HAL1_dup25212 | Uniquely | Yes | PEP: 0283 | Non | LINE | Other L1 | chr18: 45444224-45445733 | + | 120 | 1510 | SMAD2 | Proximal | 0 | Not documented | 8 |
| | | | | redundant | | | | | | | | | | | |
| L1HS_dup1463 | Multi | Yes | PEP: 0145, | Redundant | LINE | L1PA\|B\|x | chr18: 68352434-68353930 | − | 2.7 | 1497 | GTSCR1 | Distal | 34455 | Not documented | 9 |
| | | | PE | | | | | | | | | | | | |
| L1ME1_dup29500 | Multi | Yes | PEP: 0040, | Non | LINE | Other L1 | chr18: 73443999-73446189 | + | 120 | 2191 | SMIM21 | Distal | 304342 | Not documented | 550 |
| | | | PE | redundant | | | | | | | | | | | |
| L1PA4_dup11311 | Multi | Yes | PEP: 0215, | Redundant | LINE | L1PA\|B\|x | chr19: 20810588-20816745 | − | 22 | 6158 | ZNF626 | Proximal | 0 | EVE annotated | 1 |
| | | | PE | | | | | | | | | | | | |
| L1PA2_dup4699 | Multi | Yes | PEP: 0324, | Redundant | LINE | L1PA\|B\|x | chr19: 24073248-24079257 | − | 15 | 6010 | ZNF726 | Distal | 18422 | EVE annotated | 2 |
| | | | PE | | | | | | | | | | | | |
| L1PA3_dup10214 | Multi | Yes | PEP: 0145, | Redundant | LINE | L1PA\|B\|x | chr19: 24295543-24298489 | + | 17 | 2947 | ZNF254 | Proximal | 0 | EVE annotated | 3 |
| | | | PE | | | | | | | | | | | | |
| ALR/Alpha_dup1252 | Uniquely | Yes | PEP: 0300 | Non | Other | Other | chr19: 28212954-28218421 | − | 99 | 5468 | UQCRFS1 | Distal | 1479753 | Not documented | 4 |
| | | | | redundant | | Repeats | | | | | | | | | |
| SVA_F_dup917 | Multi | Yes | PEP: 0075, | Redundant | Other | SVA | chr19: 30389241-30390240 | − | 20 | 1000 | URI1 | Distal | 24312 | Not documented | 5 |
| | | | PE | | | | | | | | | | | | |
| L1PA17_dup4621 | Multi | Yes | PEP: 0313 | Non | LINE | L1PA\|B\|x | chr19: 35856441-35857924 | + | 110 | 1484 | GPR42 | Distal | 3908 | Not documented | 6 |
| | | | | redundant | | | | | | | | | | | |
| SVA_C_dup263 | Multi | Yes | PEP: 0284, | Redundant | Other | SVA | chr19: 36559267-36560956 | − | 16 | 1690 | WDR62 | Proximal | 0 | Not documented | 7 |
| | | | PE | | | | | | | | | | | | |
| HERVS71-int_dup155 | Multi | Yes | PEP: 0086 | Redundant | LTR | ERV1 | chr19: 36818249-36821384 | − | 50 | 3136 | ZFP14 | Distal | 5779 | EVE annotated | 8 |
| L1M2_dup8525 | Multi | Yes | PEP: 0190 | Non | LINE | Other L1 | chr19: 37656289-37656473 | − | 140 | 185 | ZNF585A | Proximal | 0 | Not documented | 9 |
| | | | | redundant | | | | | | | | | | | |
| LTR5_Hs_dup560 | Multi | Yes | PEP: 0261 | Redundant | LTR | ERVK | chr19: 38356817-38357784 | + | 3.6 | 968 | WDR87 | Distal | 17680 | Not documented | 560 |
| L1PA11_dup3959 | Multi | Yes | PEP: 0100 | Redundant | LINE | L1PA\|B\|x | chr19: 44473801-44478910 | + | 50 | 5110 | ZNF155 | Proximal | 0 | Not documented | 1 |
| SVA_D_dup1277 | Multi | Yes | PEP: 0075, | Redundant | Other | SVA | chr19: 46150779-46152484 | + | 18 | 1706 | EML2 | Proximal | 1893 | Not documented | 2 |
| | | | PE | | | | | | | | | | | | |
| AluYa5_dup3714 | Multi | Yes | PEP: 0044 | Redundant | SINE | Alu | chr19: 50640453-50640584 | − | 48 | 132 | IZUMO2 | Distal | 15222 | Not documented | 3 |
| AluSx1_dup102038 | Uniquely | Yes | PEP: 0006 | Redundant | SINE | Alu | chr19: 50987276-50987584 | − | 47 | 309 | EMC10 | Proximal | 669 | Not documented | 4 |
| SVA_D_dup1282 | Multi | Yes | PEP: 0075, | Redundant | Other | SVA | chr19: 52456124-52456907 | + | 14 | 784 | ZNF613 | Distal | 4113 | Not documented | 5 |
| | | | PE | | | | | | | | | | | | |
| MER21C_dup5086 | Uniquely | Yes | PEP: 0260 | Non | LTR | ERVL | chr19: 52517616-52518262 | − | 110 | 647 | ZNF614 | Proximal | 0 | Not documented | 6 |
| | | | | redundant | | | | | | | | | | | |
| SVA_E_dup217 | Multi | Yes | PEP: 0075, | Redundant | Other | SVA | chr19: 53314169-53316035 | + | 11 | 1867 | ZNF28 | Proximal | 0 | Not documented | 7 |
| | | | PE | | | | | | | | | | | | |
| SVA_E_dup220 | Multi | Yes | PEP: 0075, | Redundant | Other | SVA | chr19: 53689207-53690174 | − | 27 | 968 | ZNF665 | Proximal | 0 | Not documented | 8 |
| | | | PE | | | | | | | | | | | | |
| Tigger2a_dup3153 | Multi | Yes | PEP: 0167 | Non | DNA | DNA | chr19: 53756075-53756530 | − | 75 | 456 | ZNF677 | Proximal | 0 | Not documented | 9 |
| | | | | redundant | | | | | | | | | | | |
| L1HS_dup1486 | Multi | Yes | PEP: 0145, | Redundant | LINE | L1PA\|B\|x | chr19: 55091668-55097367 | − | 38 | 5700 | LILRA2 | Proximal | 0 | EVE annotated | 570 |
| | | | PE | | | | | | | | | | | | |
| L1PA11_dup3963 | Uniquely | Yes | PEP: 0140 | Redundant | LINE | L1PA\|B\|x | chr19: 57309708-57310995 | + | 40 | 1288 | ZIM2 | Proximal | 0 | Not documented | 1 |
| HERVL-int_dup2168 | Uniquely | Yes | PEP: 0310 | Redundant | LTR | ERVL | chr19: 57706458-57711314 | − | 30 | 4857 | ZNF264 | Proximal | 0 | EVE annotated | 2 |
| L1MEe_dup11189 | Multi/ | Yes | PEP: 0362 | Non | LINE | Other L1 | chr19: 57818984-57819694 | − | 120 | 711 | ZNF543 | Distal | 12184 | Not documented | 3 |
| | Uniquely | | | redundant | | | | | | | | | | | |
| L1PA4_dup11411 | Multi | Yes | PEP: 0219, | Redundant | LINE | L1PA\|B\|x | chr19: 58240488-58244802 | + | 19 | 4315 | AC003006.7 | Proximal | 0 | EVE annotated | 4 |
| | | | PE | | | | | | | | | | | | |
| HERVK3-int_dup297 | Multi | Yes | PEP: 0330, | Redundant | LTR | ERVK | chr19: 58822615-58824644 | + | 83 | 2030 | AC010642.1 | Proximal | 0 | EVE annotated | 5 |
| | | | PE | | | | | | | | | | | | |
| L1PA6_dup638 | Multi/ | Yes | PEP: 0145, | Redundant | LINE | L1PA\|B\|x | chr2: 104087133-104092702 | − | 34 | 5570 | TMEM182 | Distal | 626782 | EVE annotated | 6 |
| | Uniquely | | PE | | | | | | | | | | | | |
| L1MA2_dup746 | Uniquely | Yes | PEP: 0124 | Non | LINE | Other L1 | chr2: 105331200-105334955 | − | 65 | 3756 | POU3F3 | Distal | 137015 | Not documented | 7 |
| | | | | redundant | | | | | | | | | | | |
| MIR_dup21736 | Uniquely | Yes | PEP: 0049 | Non | SINE | MIR | chr2: 105953233-105953438 | + | 110 | 206 | C2orf49 | Proximal | 379 | Not documented | 8 |
| | | | | redundant | | | | | | | | | | | |
| L1HS_dup164 | Multi | Yes | PEP: 0324, | Redundant | LINE | L1PA\|B\|x | chr2: 113261390-113267422 | − | 2.3 | 6033 | TTL | Proximal | 0 | EVE annotated | 9 |
| | | | PE | | | | | | | | | | | | |
| L2_dup7269 | Multi | Yes | PEP: 0067 | Redundant | LINE | L2 | chr2: 114383106-114383268 | + | 130 | 163 | RABLZA | Proximal | 1539 | Not documented | 580 |
| AluJb_dup18789 | Multi | Yes | PEP: 0224, | Redundant | SINE | Alu | chr2: 128604936-128605224 | + | 79 | 289 | POLR2D | Proximal | 0 | Not documented | 1 |
| | | | PE | | | | | | | | | | | | |
| L1PA3_dup1176 | Multi | Yes | PEP: 0145, | Redundant | LINE | L1PA\|B\|x | chr2: 132957414-132959879 | + | 12 | 2466 | ANKRD30BL | Proximal | 0 | EVE annotated | 2 |
| | | | PE | | | | | | | | | | | | |
| SVA_D_dup192 | Multi | Yes | PEP: 0233 | Redundant | Other | SVA | chr2: 132977958-132978887 | + | 20 | 930 | ANKRD30BL | Proximal | 0 | Not documented | 3 |
| L1PA3_dup1177 | Multi | Yes | PEP: 0324, | Redundant | LINE | L1PA\|B\|x | chr2: 132988899-132994855 | + | 15 | 5957 | ANKRD30BL | Proximal | 0 | EVE annotated | 4 |
| | | | PE | | | | | | | | | | | | |
| L1PA4_dup1338 | Multi | Yes | PEP: 0219, | Redundant | LINE | L1PA\|B\|x | chr2: 133286455-133292577 | + | 20 | 6123 | GPR39 | Proximal | 0 | EVE annotated | 5 |
| | | | PE | | | | | | | | | | | | |
| L1PA7_dup1431 | Multi | Yes | PEP: 0053, | Redundant | LINE | L1PA\|B\|x | chr2: 142199999-142206420 | − | 46 | 6422 | LRP1B | Proximal | 0 | EVE annotated | 6 |
| | | | PE | | | | | | | | | | | | |
| L2a_dup23251 | Uniquely | Yes | PEP: 0262, | Non | LINE | L2 | chr2: 142661213-142663222 | − | 160 | 2010 | LRP1B | Proximal | 0 | Not documented | 7 |
| | | | PE | redundant | | | | | | | | | | | |
| L1PA7_dup1433 | Multi | Yes | PEP: 0163 | Redundant | LINE | L1PA\|B\|x | chr2: 142772780-142773280 | + | 37 | 501 | LRP1B | Proximal | 0 | Not documented | 8 |
| L1HS_dup179 | Multi | Yes | PEP: 0210, | Redundant | LINE | L1PA\|B\|x | chr2: 144618526-144624556 | + | 2.3 | 6031 | GTDC1 | Distal | 71080 | EVE annotated | 9 |
| | | | PE | | | | | | | | | | | | |
| L1HS_dup181 | Multi | Yes | PEP: 0324, | Redundant | LINE | L1PA\|B\|x | chr2: 153864281-153870310 | − | 3.2 | 6030 | ARL6IP6 | Distal | 246515 | EVE annotated | 590 |
| | | | PE | | | | | | | | | | | | |
| L1PA8A_dup203 | Uniquely | Yes | PEP: 0185 | Redundant | LINE | L1PA\|B\|x | chr2: 15484374-15487251 | + | 41 | 2878 | NBAS | Proximal | | EVE annotated | 1 |
| L1PA4_dup1483 | Uniquely | Yes | PEP: 0113 | Redundant | LINE | L1PA\|B\|x | chr2: 169723779-169727875 | + | 16 | 4097 | SPC25 | Proximal | 0 | EVE annotated | 2 |
| L1HS_dup192 | Multi | Yes | PEP: 0324, | Redundant | LINE | L1PA\|B\|x | chr2: 170105134-170111166 | + | 2.3 | 6033 | LRP2 | Proximal | 0 | EVE annotated | 3 |
| | | | PE | | | | | | | | | | | | |
| L1HS_dup194 | Multi | Yes | PEP: 0324, | Redundant | LINE | L1PA\|B\|x | chr2: 176346694-176352722 | − | 2.3 | 6029 | ATP5G3 | Distal | 297360 | EVE annotated | 4 |
| | | | PE | | | | | | | | | | | | |
| L1PA6_dup790 | Uniquely | Yes | PEP: 0303 | Redundant | LINE | L1PA\|B\|x | chr2: 179216841-179222974 | + | 34 | 6134 | OSBPL6 | Proximal | 0 | EVE annotated | 5 |
| L1HS_dup206 | Multi | Yes | PEP: 0324, | Redundant | LINE | L1PA\|B\|x | chr2: 197770312-197776343 | + | 1.8 | 6032 | PGAP1 | Proximal | 0 | EVE annotated | 6 |
| | | | PE | | | | | | | | | | | | |
| AluSz6_dup6483 | Multi | Yes | PEP: 0008 | Redundant | SINE | Alu | chr2: 198339128-198339427 | + | 49 | 300 | COQ10B | Proximal | 0 | Not documented | 7 |
| L1PA7_dup1817 | Uniquely | Yes | PEP: 0002 | Non | LINE | L1PA\|B\|x | chr2: 210352720-210359164 | + | 42 | 6445 | MAP2 | Proximal | 0 | EVE annotated | 8 |
| | | | | redundant | | | | | | | | | | | |
| L1HS_dup122 | Multi | Yes | PEP: 0324, | Redundant | LINE | L1PA\|B\|x | chr2: 214431956-214437986 | − | 3.6 | 6031 | SPAG16 | Proximal | 0 | EVE annotated | 9 |
| | | | PE | | | | | | | | | | | | |
| AluSx_dup21091 | Multi | Yes | PEP: 0132 | Redundant | SINE | Alu | chr2: 224831952-224832249 | − | 52 | 298 | MRPL44 | Proximal | 0 | Not documented | 600 |
| L1PA6_dup514 | Multi | Yes | PEP: 0324, | Redundant | LINE | L1PA\|B\|x | chr2: 24318028-24324128 | + | 29 | 6101 | FAM228B | Proximal | 0 | EVE annotated | 1 |
| | | | PE | | | | | | | | | | | | |
| AluSq10_dup194 | Uniquely | Yes | PEP: 0321 | Redundant | SINE | Alu | chr2: 27099830-27100133 | − | 28 | 304 | DPYSL5 | Proximal | 0 | Not documented | 2 |
| L1HS_dup126 | Multi | Yes | PEP: 0324, | Redundant | LINE | L1PA\|B\|x | chr2: 4781320-4787350 | + | 1.4 | 6031 | DCDC2C | Distal | 945199 | EVE annotated | 3 |
| | | | PE | | | | | | | | | | | | |
| L1PA2_dup376 | Multi | Yes | PEP: 0210, | Redundant | LINE | L1PA\|B\|x | chr2: 49235167-49238753 | − | 10 | 3587 | FSHR | Proximal | 0 | EVE annotated | 4 |
| | | | PE | | | | | | | | | | | | |
| L1PA6_dup549 | Multi | Yes | PEP: 0219 | Redundant | LINE | L1PA\|B\|x | chr2: 49454726-49460932 | − | 41 | 6207 | FSHR | Distal | 73051 | EVE annotated | 5 |
| AluJr_dup7585 | Uniquely | Yes | PEP: 0292 | Non | SINE | Alu | chr2: 51099809-51100150 | + | 72 | 342 | NRXN1 | Proximal | 0 | Not documented | 6 |
| | | | | redundant | | | | | | | | | | | |
| L1MA2_dup599 | Uniquely | Yes | PEP: 0155 | Redundant | LINE | Other L1 | chr2: 51616498-51618520 | − | 81 | 2023 | NRXN1 | Distal | 356825 | Not documented | 7 |
| L1PA7_dup1105 | Multi/ | Yes | PEP: 0303 | Redundant | LINE | L1PA\|B\|x | chr2: 51618536-51624774 | + | 52 | 6239 | NRXN1 | Distal | 358863 | EVE annotated | 8 |
| | Uniquely | | | | | | | | | | | | | | |
| L1PA2_dup398 | Multi/ | Yes | PEP: 0324, | Redundant | LINE | L1PA\|B\|x | chr2: 63062614-63068633 | + | 9.1 | 6020 | EHBP1 | Proximal | 0 | EVE annotated | 9 |
| | Uniquely | | PE | | | | | | | | | | | | |
| L1ME4a_dup4569 | Multi | Yes | PEP: 0214, | Redundant | LINE | Other L1 | chr2: 70131996-70132774 | − | 130 | 779 | MXD1 | Proximal | 0 | Not documented | 610 |
| | | | PE | | | | | | | | | | | | |
| L1HS_dup150 | Multi | Yes | PEP: 0145, | Redundant | LINE | L1PA\|B\|x | chr2: 81834791-81836329 | − | 3.6 | 1539 | CTNNA2 | Distal | 958887 | Not documented | 1 |
| | | | PE | | | | | | | | | | | | |
| L1HS_dup155 | Multi | Yes | PEP: 0324, | Redundant | LINE | L1PA\|B\|x | chr2: 86882362-86888391 | − | 2.7 | 6030 | RNF103-CHMP3 | Proximal | 0 | EVE annotated | 2 |
| | | | PE | | | | | | | | | | | | |
| L1HS_dup157 | Multi | Yes | PEP: 0210, | Redundant | LINE | L1PA\|B\|x | chr2: 89029231-89032287 | + | 2.3 | 3057 | RPIA | Proximal | 0 | EVE annotated | 3 |
| | | | PE | | | | | | | | | | | | |
| L1MC_dup1182 | Multi | Yes | PEP: 0125 | Redundant | LINE | Other L1 | chr2: 90280090-90280485 | + | 140 | 396 | CH17-132F21.1 | Distal | 177717 | Not documented | 4 |
| L1M4_dup1621 | Multi | Yes | PEP: 0125 | Redundant | LINE | Other L1 | chr2: 91754408-91754815 | − | 140 | 408 | RP11-685N3.1 | Distal | 1216012 | Not documented | 5 |
| L1M5_dup61233 | Multi | Yes | PEP: 0350 | Non | LINE | Other L1 | chr20: 26031358-26031842 | + | 120 | 485 | FAM182B | Distal | 182498 | Not documented | 6 |
| | | | | redundant | | | | | | | | | | | |
| L1MA4A_dup6060 | Multi/ | Yes | PEP: 0365 | Non | LINE | Other L1 | chr20: 39819111-39821807 | − | 89 | 2697 | ZHX3 | Proximal | 0 | Not documented | 7 |
| | Uniquely | | | redundant | | | | | | | | | | | |
| MIRb_dup216177 | Uniquely | Yes | PEP: 0081 | Redundant | SINE | MIR | chr20: 50173854-50174018 | + | 130 | 165 | NFATC2 | Proximal | 0 | Not documented | 8 |
| MLT1H_dup9158 | Multi | Yes | PEP: 0353 | Non | LTR | ERVL- | chr20: 628009-628491 | + | 100 | 483 | SRXN1 | Proximal | 0 | Not documented | 9 |
| | | | | redundant | | MaLR | | | | | | | | | |
| MIRb_dup211037 | Multi | Yes | PEP: 0222 | Non | SINE | MIR | chr20: 629090-629241 | − | 170 | 152 | SRXN1 | Proximal | 0 | Not documented | 620 |
| | | | | redundant | | | | | | | | | | | |
| L1HS_dup1490 | Multi | Yes | PEP: 0324, | Redundant | LINE | L1PA\|B\|x | chr20: 7096829-7102846 | − | 2.7 | 6018 | BMP2 | Distal | 335903 | EVE annotated | 1 |
| | | | PE | | | | | | | | | | | | |
| L1PA7_dup12693 | Uniquely | Yes | PEP: 0052 | Redundant | LINE | L1PA\|B\|x | chr21: 17596650-17603025 | + | 44 | 6376 | USP25 | Distal | 344274 | Not documented | 2 |
| L1PA2_dup4819 | Multi | Yes | PEP: 0145, | Redundant | LINE | L1PA\|B\|x | chr21: 27290680-27296704 | + | 5.5 | 6025 | APP | Proximal | 0 | EVE annotated | 3 |
| | | | PE | | | | | | | | | | | | |
| L1PA3_dup10425 | Multi | Yes | PEP: 0324, | Redundant | LINE | L1PA\|B\|x | chr21: 9656297-9662430 | − | 26 | 6134 | TPTE | Distal | 1243772 | EVE annotated | 4 |
| | | | PE | | | | | | | | | | | | |
| SATR1_dup508 | Multi | Yes | PEP: 0041 | Redundant | Other | Other | chr21: 9732221-9733136 | + | 85 | 916 | TPTE | Distal | 1173066 | Not documented | 5 |
| | | | | | | Repeats | | | | | | | | | |
| TAR1_dup146 | Multi | Yes | PEP: 0269 | Redundant | Other | Other | chr21: 9881896-9882569 | + | 77 | 674 | TPTE | Distal | 1023633 | Not documented | 6 |
| | | | | | | Repeats | | | | | | | | | |
| L1PA7_dup12670 | Multi | Yes | PEP: 0168, | Redundant | LINE | L1PA\|B\|x | chr21: 9976908-9982108 | − | 42 | 5201 | TPTE | Distal | 924094 | EVE annotated | 7 |
| | | | PE | | | | | | | | | | | | |
| HERVK9-int_dup632 | Multi | Yes | PEP: 0147 | Redundant | LTR | ERVK | chr21: 9989864-9992670 | − | 32 | 2807 | TPTE | Distal | 913532 | EVE annotated | 8 |
| CER_dup42 | Multi | Yes | PEP: 0266 | Redundant | Other | Other | chr22: 16506892-16608196 | − | 67 | 101305 | OR11H1 | Distal | 57088 | Not documented | 9 |
| | | | | | | Repeats | | | | | | | | | |
| CER_dup43 | Multi | Yes | PEP: 0069 | Redundant | Other | Other | chr22: 16608226-16626569 | + | 65 | 18344 | OR11H1 | Distal | 158422 | Not documented | 630 |
| | | | | | | Repeats | | | | | | | | | |
| L1PA5_dup11139 | Uniquely | Yes | PEP: 0116, | Redundant | LINE | L1PA\|B\|x | chr22: 28924936-28931063 | + | 30 | 6128 | TTC28 | Proximal | 0 | EVE annotated | 1 |
| | | | PE | | | | | | | | | | | | |
| L1HS_dup1524 | Multi | Yes | PEP: 0324, | Redundant | LINE | L1PA\|B\|x | chr22: 29059272-29065303 | + | 1.8 | 6032 | TTC28 | Proximal | 0 | EVE annotated | 7 |
| | | | PE | | | | | | | | | | | | |
| L1M2c_dup430 | Uniquely | Yes | PEP: 0217 | Non | LINE | Other L1 | chr22: 38767606-38771715 | − | 98 | 4110 | CSNK1E | Proximal | 0 | Not documented | 3 |
| | | | | redundant | | | | | | | | | | | |
| L1HS_dup1527 | Multi | Yes | PEP: 0324, | Redundant | LINE | L1PA\|B\|x | chr22: 49381574-49387604 | − | 1.8 | 6031 | FAM19A5 | Distal | 134851 | EVE annotated | 4 |
| | | | PE | | | | | | | | | | | | |
| L2_dup56034 | Multi | Yes | PEP: 0067 | Redundant | LINE | L2 | chr22: 51223639-51223803 | − | 120 | 165 | RABL2B | Proximal | 1549 | Not documented | 5 |
| L1HS_dup288 | Multi | Yes | PEP: 0324, | Redundant | LINE | L1PA\|B\|x | chr3: 108468248-108474272 | + | 2.7 | 6025 | RETNLB | Proximal | 0 | EVE annotated | 6 |
| | | | PE | | | | | | | | | | | | |
| L1MB1_dup1023 | Uniquely | Yes | PEP: 0258 | Redundant | LINE | Other L1 | chr3: 115639412-115641599 | − | 110 | 2188 | LSAMP | Proximal | 0 | Not documented | 7 |
| L1PA2_dup900 | Multi/ | Yes | PEP: 0324, | Redundant | LINE | L1PA\|B\|x | chr3: 115922233-115928273 | − | 12 | 6041 | LSAMP | Proximal | 0 | EVE annotated | 8 |
| | Uniquely | | PE | | | | | | | | | | | | |
| L1HS_dup296 | Multi | Yes | PEP: 0324, | Redundant | LINE | L1PA\|B\|x | chr3: 116078847-116084873 | + | 1.8 | 6027 | LSAMP | Proximal | 0 | EVE annotated | 9 |
| | | | PE | | | | | | | | | | | | |
| SVA_D_dup294 | Multi | Yes | PEP: 0284, | Redundant | Other | SVA | chr3: 116768855-116770341 | + | 16 | 1487 | LSAMP | Proximal | 0 | Not documented | 640 |
| | | | PE | | | | | | | | | | | | |
| L1HS_dup298 | Multi | Yes | PEP: 0324, | Redundant | LINE | L1PA\|B\|x | chr3: 120291869-120298033 | + | 10 | 6165 | NDUFB4 | Distal | 17124 | EVE annotated | 1 |
| | | | PE | | | | | | | | | | | | |
| AluSz_dup18468 | Multi | Yes | PEP: 0074 | Redundant | SINE | Alu | chr3: 120406667-120406937 | − | 44 | 271 | RABL3 | Proximal | 0 | Not documented | 2 |
| L1P2_dup323 | Uniquely | Yes | PEP: 0113, | Redundant | LINE | L1PA\|B\|x | chr3: 125715097-125720650 | + | 42 | 5554 | SLC41A3 | Distal | 4549 | Not documented | 3 |
| | | | PE | | | | | | | | | | | | |
| L1MC2_dup1267 | Uniquely | Yes | PEP: 0171 | Redundant | LINE | Other L1 | chr3: 125893173-125896600 | + | 74 | 3428 | ALDH1L1 | Proximal | 0 | EVE annotated | 4 |
| L1HS_dup306 | Multi | Yes | PEP: 0324, | Redundant | LINE | L1PA\|B\|x | chr3: 130347653-130352909 | + | 1.4 | 5257 | COL6A6 | Proximal | 0 | EVE annotated | 5 |
| | | | PE | | | | | | | | | | | | |
| L1M1_dup1725 | Uniquely | Yes | PEP: 0119 | Non | LINE | Other L1 | chr3: 139134334-139138647 | − | 61 | 4314 | COPB2 | Distal | 25761 | Not documented | 6 |
| | | | | redundant | | | | | | | | | | | |
| L1PA5_dup2212 | Multi | Yes | PEP: 0324, | Redundant | LINE | L1PA\|B\|x | chr3: 142193948-142200076 | − | 25 | 6129 | ATR | Proximal | 0 | EVE annotated | 7 |
| | | | PE | | | | | | | | | | | | |
| MLT1F1_dup677 | Multi | Yes | PEP: 0325 | Non | LTR | ERVL- | chr3: 147724158-147724663 | + | 99 | 506 | ZIC1 | Distal | 496079 | Not documented | 8 |
| | | | | redundant | | MaLR | | | | | | | | | |
| HSMAR1_dup117 | Multi | Yes | PEP: 0090 | Redundant | DNA | DNA | chr3: 14986580-14987797 | − | 44 | 1218 | NR2C2 | Proximal | 1295 | Not documented | 9 |
| L1PA5_dup2263 | Multi/ | Yes | PEP: 0303 | Redundant | LINE | L1PA\|B\|x | chr3: 153980395-153986513 | + | 32 | 6119 | DHX36 | Distal | 3823 | EVE annotated | 650 |
| | Uniquely | | | | | | | | | | | | | | |
| LTR12C_dup547 | Uniquely | Yes | PEP: 0212 | Non | LTR | ERV1 | chr3: 156458184-156459561 | + | 29 | 1378 | TIPARP | Distal | 33626 | Not documented | 1 |
| | | | | redundant | | | | | | | | | | | |
| L1PBa_dup493 | Uniquely | Yes | PEP: 0275 | Non | LINE | L1PA\|B\|x | chr3: 165164058-165167033 | + | 53 | 2976 | SLITRK3 | Distal | 249162 | Not documented | 2 |
| | | | | redundant | | | | | | | | | | | |
| AluSz_dup19789 | Multi | Yes | PEP: 0097 | Redundant | SINE | Alu | chr3: 169806672-169806972 | + | 60 | 301 | PHC3 | Proximal | 0 | Not documented | 3 |
| Tigger1_dup2738 | Multi/ | Yes | PEP: 0154 | Redundant | DNA | DNA | chr3: 173599487-173600791 | − | 68 | 1305 | NLGN1 | Proximal | 0 | Not documented | 4 |
| | Uniquely | | | | | | | | | | | | | | |
| L4_dup3783 | Multi | Yes | PEP: 0115 | Non | LINE | RTE | chr3: 173687002-173687725 | − | 170 | 724 | NLGN1 | Proximal | 0 | Not documented | 5 |
| | | | | redundant | | | | | | | | | | | |
| MamRep605_dup1076 | Uniquely | Yes | PEP: 0036 | Non | Other | Other | chr3: 173706880-173707391 | − | 140 | 512 | NLGN1 | Proximal | 0 | Not documented | 6 |
| | | | | redundant | | Repeats | | | | | | | | | |
| L2a_dup39168 | Uniquely | Yes | PEP: 0173 | Non | LINE | L2 | chr3: 174160395-174162124 | − | 120 | 1730 | NAALADL2 | Proximal | 0 | Not documented | 7 |
| | | | | redundant | | | | | | | | | | | |
| AluSp_dup10330 | Multi | Yes | PEP: 0340 | Redundant | SINE | Alu | chr3: 179042800-179043107 | − | 53 | 308 | ZNF639 | Proximal | 0 | Not documented | 8 |
| AluSz6_dup9770 | Multi | Yes | PEP: 0043 | Redundant | SINE | Alu | chr3: 183890240-183890543 | + | 55 | 304 | DVL3 | Proximal | 0 | Not documented | 9 |
| L1PA13_dup1706 | Uniquely | Yes | PEP: 0186 | Redundant | LINE | L1PA\|B\|x | chr3: 184816903-184819238 | + | 43 | 2336 | C3orf70 | Proximal | 0 | Not documented | 660 |
| SVA_D_dup334 | Multi | Yes | PEP: 0284, | Redundant | Other | SVA | chr3: 197328597-197330107 | − | 11 | 1511 | BDH1 | Distal | 28404 | Not documented | 1 |
| | | | PE | | | | | | | | | | | | |
| L1PA7_dup2008 | Uniquely | Yes | PEP: 0209 | Redundant | LINE | L1PA\|B\|x | chr3: 21596709-21603100 | − | 44 | 6392 | ZNF385D | Proximal | 0 | Not documented | 2 |
| L2a_dup31321 | Multi | Yes | PEP: 0354 | Non | LINE | L2 | chr3: 38538147-38538440 | + | 130 | 294 | EXOG | Proximal | 0 | Not documented | 3 |
| | | | | redundant | | | | | | | | | | | |
| L2a_dup29030 | Uniquely | Yes | PEP: 0165 | Non | LINE | L2 | chr3: 3913399-3915817 | + | 120 | 2419 | SUMF1 | Proximal | 0 | Not documented | 4 |
| | | | | redundant | | | | | | | | | | | |
| L1PA4_dup1769 | Uniquely | Yes | PEP: 0303 | Redundant | LINE | L1PA\|B\|x | chr3: 3951154-3956626 | + | 19 | 5473 | SUMF1 | Proximal | 0 | EVE annotated | 5 |
| Tigger1_dup2151 | Multi/ | Yes | PEP: 0182 | Non | DNA | DNA | chr3: 40580015-40581111 | + | 55 | 1097 | ZNF621 | Proximal | 0 | Not documented | 6 |
| | Uniquely | | | redundant | | | | | | | | | | | |
| L1PA2_dup774 | Multi/ | Yes | PEP: 0324, | Redundant | LINE | L1PA\|B\|x | chr3: 43106267-43112282 | + | 6.4 | 6016 | FAM198A | Distal | 4565 | EVE annotated | 7 |
| | Uniquely | | PE | | | | | | | | | | | | |
| L1P2_dup267 | Uniquely | Yes | PEP: 0244, | Redundant | LINE | L1PA\|B\|x | chr3: 8106444-8110425 | − | 36 | 3982 | GRM7 | Distal | 323230 | EVE annotated | 8 |
| | | | PE | | | | | | | | | | | | |
| L1HS_dup273 | Multi | Yes | PEP: 0145, | Redundant | LINE | L1PA\|B\|x | chr3: 83853136-83855348 | − | 2.7 | 2213 | CADM2 | Distal | 1152785 | EVE annotated | 9 |
| | | | PE | | | | | | | | | | | | |
| L1HS_dup274 | Multi | Yes | PEP: 0169, | Redundant | LINE | L1PA\|B\|x | chr3: 89509976-89516006 | − | 1.8 | 6031 | EPHA3 | Proximal | 0 | EVE annotated | 670 |
| | | | PE | | | | | | | | | | | | |
| Tigger1_dup2026 | Multi | Yes | PEP: 0079 | Non | DNA | DNA | chr3: 9433242-9433517 | − | 55 | 276 | THUMPD3 | Distal | 4768 | Not documented | 1 |
| | | | | redundant | | | | | | | | | | | |
| SVA_D_dup371 | Multi | Yes | PEP: 0284, | Redundant | Other | SVA | chr4: 113878170-113879804 | − | 10 | 1635 | ANK2 | Proximal | 0 | Not documented | 2 |
| | | | PE | | | | | | | | | | | | |
| L2a_dup48093 | Multi/ | Yes | PEP: 0114 | Non | LINE | L2 | chr4: 114369874-114370755 | − | 120 | 882 | CAMK2D | Proximal | 1434 | Not documented | 3 |
| | Uniquely | | | redundant | | | | | | | | | | | |
| L1PA2_dup1276 | Multi | Yes | PEP: 0324, | Redundant | LINE | L1PA\|B\|x | chr4: 114410295-114416323 | − | 12 | 6029 | CAMK2D | Proximal | 0 | EVE annotated | 4 |
| | | | PE | | | | | | | | | | | | |
| L1HS_dup447 | Multi | Yes | PEP: 0145, | Redundant | LINE | L1PA\|B\|x | chr4: 117125010-117126599 | − | 3.6 | 1590 | MTRNR2L13 | Distal | 93418 | Not documented | 5 |
| | | | PE | | | | | | | | | | | | |
| AluSz_dup23620 | Multi | Yes | PEP: 0008 | Redundant | SINE | Alu | chr4: 120218610-120218911 | − | 57 | 302 | C4orf3 | Proximal | 0 | Not documented | 6 |
| X9_LINE_dup23 | Multi | Yes | PEP: 0150, | Non | LINE | Other L1 | chr4: 1235081-1235197 | − | 110 | 117 | CTBP1 | Proximal | 0 | Not documented | 7 |
| | | | PE | redundant | | | | | | | | | | | |
| L1PA4_dup3230 | Uniquely | Yes | PEP: 0113 | Redundant | LINE | L1PA\|B\|x | chr4: 125533093-125539240 | − | 22 | 6148 | ANKRD50 | Distal | 45968 | EVE annotated | 8 |
| L1PA3_dup2903 | Uniquely | Yes | PEP: 0303 | Redundant | LINE | L1PA\|B\|x | chr4: 128597764-128603911 | + | 16 | 6148 | INTU | Proximal | 0 | EVE annotated | 9 |
| L1HS_dup469 | Multi | Yes | PEP: 0210, | Redundant | LINE | L1PA\|B\|x | chr4: 137214650-137220701 | − | 0.91 | 6052 | PCDH18 | Distal | 1219372 | EVE annotated | 680 |
| | | | PE | | | | | | | | | | | | |
| L1HS_dup470 | Multi | Yes | PEP: 0210, | Redundant | LINE | L1PA\|B\|x | chr4: 139468878-139473208 | + | 1.4 | 4331 | SLC7A11 | Distal | 305376 | EVE annotated | 1 |
| | | | PE | | | | | | | | | | | | |
| L1PA7_dup3717 | Multi/ | Yes | PEP: 0053, | Redundant | LINE | L1PA\|B\|x | chr4: 151192065-151198580 | + | 38 | 6516 | LRBA | Proximal | 0 | EVE annotated | 2 |
| | Uniquely | | PE | | | | | | | | | | | | |
| L1HS_dup354 | Multi | Yes | PEP: 0324, | Redundant | LINE | L1PA\|B\|x | chr4: 15843170-15849195 | + | 1.4 | 6026 | CD38 | Proximal | 0 | EVE annotated | 3 |
| | | | PE | | | | | | | | | | | | |
| L1HS_dup490 | Multi | Yes | PEP: 0324, | Redundant | LINE | L1PA\|B\|x | chr4: 167677047-167683059 | + | 1.8 | 6013 | SPOCK3 | Proximal | 0 | EVE annotated | 4 |
| | | | PE | | | | | | | | | | | | |
| L1PA16_dup4108 | Uniquely | Yes | PEP: 0187 | Non | LINE | L1PA\|B\|x | chr4: 177283792-177290564 | − | 65 | 6773 | SPCS3 | Distal | 30397 | Not documented | 5 |
| | | | | redundant | | | | | | | | | | | |
| L1M4c_dup1231 | Uniquely | Yes | PEP: 0205 | Redundant | LINE | Other L1 | chr4: 1966001-1967336 | + | 69 | 1336 | WHSC1 | Proximal | 0 | Not documented | 6 |
| L1HS_dup359 | Multi | Yes | PEP: 0210, | Redundant | LINE | L1PA\|B\|x | chr4: 21161014-21167044 | + | 1.4 | 6031 | KCNIP4 | Proximal | 0 | EVE annotated | 7 |
| | | | PE | | | | | | | | | | | | |
| AluSz6_dup10521 | Multi | Yes | PEP: 0092 | Redundant | SINE | Alu | chr4: 41960718-41961033 | − | 45 | 316 | TMEM33 | Proximal | 0 | Not documented | 8 |
| L1HS_dup383 | Multi | Yes | PEP: 0231 | Redundant | LINE | L1PA\|B\|x | chr4: 48736862-48738001 | + | 3.6 | 1140 | FRYL | Proximal | 0 | Not documented | 9 |
| L1P1_dup714 | Multi | Yes | PEP: 0324, | Redundant | LINE | L1PA\|B\|x | chr4: 49622935-49628634 | + | 26 | 5700 | CWH43 | Distal | 558838 | EVE annotated | 690 |
| | | | PE | | | | | | | | | | | | |
| AluSc_dup8059 | Multi | Yes | PEP: 0338 | Redundant | SINE | Alu | chr4: 52887543-52887839 | + | 35 | 297 | SGCB | Proximal | 0 | Not documented | 1 |
| L1PA4_dup2819 | Uniquely | Yes | PEP: 0303 | Redundant | LINE | L1PA\|B\|x | chr4: 53761344-53767458 | − | 23 | 6115 | SCFD2 | Proximal | 0 | EVE annotated | 2 |
| L1M5_dup15476 | Uniquely | Yes | PEP: 0134 | Redundant | LINE | Other L1 | chr4: 54900584-54902447 | − | 120 | 1864 | CHIC2 | Proximal | 0 | Not documented | 3 |
| L1PA3_dup2546 | Uniquely | Yes | PEP: 0113, | Redundant | LINE | L1PA\|B\|x | chr4: 57197421-57203548 | − | 16 | 6128 | AASDH | Proximal | 909 | EVE annotated | 4 |
| | | | PE | | | | | | | | | | | | |
| L1HS_dup388 | Multi | Yes | PEP: 0324, | Redundant | LINE | L1PA\|B\|x | chr4: 58428483-58434513 | − | 3.6 | 6031 | IGFBP7 | Distal | 451933 | EVE annotated | 5 |
| | | | PE | | | | | | | | | | | | |
| L1HS_dup400 | Multi | Yes | PEP: 0324, | Redundant | LINE | L1PA\|B\|x | chr4: 62805646-62811680 | − | 2.3 | 6035 | LPHN3 | Proximal | 0 | EVE annotated | 6 |
| | | | PE | | | | | | | | | | | | |
| MLT1H-int_dup171 | Multi | Yes | PEP: 0318 | Non | LTR | ERVL- | chr4: 6672272-6672596 | − | 130 | 325 | AC093323.1 | Distal | 21187 | Not documented | 7 |
| | | | | redundant | | MaLR | | | | | | | | | |
| LTR12C_dup611 | Multi | Yes | PEP: 0250 | Redundant | LTR | ERV1 | chr4: 6674268-6675474 | + | 29 | 1207 | AC093323.1 | Distal | 18309 | Not documented | 8 |
| L1PA5_dup2725 | Multi | Yes | PEP: 0210, | Redundant | LINE | L1PA\|B\|x | chr4: 70101875-70104864 | + | 29 | 2990 | UGT2B11 | Distal | 21427 | EVE annotated | 9 |
| | | | PE | | | | | | | | | | | | |
| L1PA5_dup2727 | Multi | Yes | PEP: 0100 | Redundant | LINE | L1PA\|B\|x | chr4: 70206700-70208894 | + | 36 | 2195 | UGT2B28 | Distal | 45933 | EVE annotated | 700 |
| L1HS_dup406 | Multi | Yes | PEP: 0210, | Redundant | LINE | L1PA\|B\|x | chr4: 71194624-71200024 | + | 1.8 | 5401 | CABS1 | Proximal | 660 | EVE annotated | 1 |
| | | | PE | | | | | | | | | | | | |
| L1PA8_dup1944 | Uniquely | Yes | PEP: 0303 | Redundant | LINE | L1PA\|B\|x | chr4: 72174145-72180662 | + | 44 | 6518 | SLC4A4 | Proximal | 0 | EVE annotated | 2 |
| L1PA3_dup2625 | Uniquely | Yes | PEP: 0113, | Redundant | LINE | L1PA\|B\|x | chr4: 72285547-72290720 | + | 14 | 5174 | SLC4A4 | Proximal | 0 | EVE annotated | 3 |
| | | | PE | | | | | | | | | | | | |
| L1HS_dup412 | Multi | Yes | PEP: 0324, | Redundant | LINE | L1PA\|B\|x | chr4: 80858870-80864900 | + | 1.4 | 6031 | ANTXR2 | Proximal | 0 | EVE annotated | 4 |
| | | | PE | | | | | | | | | | | | |
| L1PA2_dup1208 | Multi/ | Yes | PEP: 0324, | Redundant | LINE | L1PA\|B\|x | chr4: 86991544-86997572 | − | 10 | 6029 | MAPK10 | Proximal | 0 | EVE annotated | 5 |
| | Uniquely | | PE | | | | | | | | | | | | |
| L1HS_dup425 | Multi | Yes | PEP: 0324, | Redundant | LINE | L1PA\|B\|x | chr4: 88268276-88274298 | − | 1.4 | 6023 | HSD17B11 | Proximal | 0 | EVE annotated | 6 |
| | | | PE | | | | | | | | | | | | |
| L1HS_dup429 | Multi | Yes | PEP: 0324, | Redundant | LINE | L1PA\|B\|x | chr4: 91596878-91602908 | − | 1.8 | 6031 | CCSER1 | Proximal | 0 | EVE annotated | 7 |
| | | | PE | | | | | | | | | | | | |
| L1PB2_dup704 | Uniquely | Yes | PEP: 0358, | Redundant | LINE | L1PA\|B\|x | chr4: 95955442-95957284 | − | 49 | 1843 | BMPR1B | Proximal | 0 | Not documented | 8 |
| | | | PE | | | | | | | | | | | | |
| L1HS_dup436 | Multi | Yes | PEP: 0324, | Redundant | LINE | L1PA\|B\|x | chr4: 99513587-99519614 | − | 2.7 | 6028 | TSPAN5 | Proximal | 0 | EVE annotated | 9 |
| | | | PE | | | | | | | | | | | | |
| L1HS_dup562 | Multi | Yes | PEP: 0145, | Redundant | LINE | L1PA\|B\|x | chr5: 103854289-103860317 | + | 1.4 | 6029 | NUDT12 | Distal | 955796 | EVE annotated | 710 |
| | | | PE | | | | | | | | | | | | |
| SVA_F_dup311 | Multi | Yes | PEP: 0075, | Redundant | Other | SVA | chr5: 108678238-108678978 | + | 31 | 741 | PJA2 | Proximal | 0 | Not documented | 1 |
| | | | PE | | | | | | | | | | | | |
| AluSx_dup39137 | Multi/ | Yes | PEP: 0164, | Redundant | SINE | Alu | chr5: 112104738-112105047 | − | 45 | 310 | APC | Proximal | 0 | Not documented | 2 |
| | Uniquely | | PE | | | | | | | | | | | | |
| MER34A_dup551 | Multi | Yes | PEP: 0291, | Non | LTR | ERV1 | chr5: 127396766-127397217 | + | 120 | 452 | SLC12A2 | Distal | 22242 | Not documented | 3 |
| | | | PE | redundant | | | | | | | | | | | |
| AluSq2_dup15956 | Multi | Yes | PEP: 0340 | Redundant | SINE | Alu | chr5: 133737778-133738093 | − | 53 | 316 | CDKNZAIPNL | Proximal | 0 | Not documented | 4 |
| SVA_E_dup94 | Multi | Yes | PEP: 0075, | Redundant | Other | SVA | chr5: 137022578-137023848 | + | 28 | 1271 | KLHL3 | Proximal | 0 | Not documented | 5 |
| | | | PE | | | | | | | | | | | | |
| L2c_dup49860 | Multi | Yes | PEP: 0285 | Non | LINE | L2 | chr5: 140599064-140600044 | + | 170 | 981 | PCDHB13 | Distal | 2072 | Not documented | 6 |
| | | | | redundant | | | | | | | | | | | |
| AluSx4_dup1785 | Multi/ | Yes | PEP: 0074 | Redundant | SINE | Alu | chr5: 140860368-140860654 | + | 54 | 287 | PCDHGC3 | Proximal | 0 | Not documented | 7 |
| | Uniquely | | | | | | | | | | | | | | |
| L2a_dup60322 | Uniquely | Yes | PEP: 0065 | Non | LINE | L2 | chr5: 146093041-146094112 | − | 120 | 1072 | PPP2R2B | Proximal | 0 | Not documented | 8 |
| | | | | redundant | | | | | | | | | | | |
| L2b_dup33029 | Uniquely | Yes | PEP: 0246 | Non | LINE | L2 | chr5: 146431875-146432410 | − | 140 | 536 | PPP2R2B | Proximal | 0 | Not documented | 9 |
| | | | | redundant | | | | | | | | | | | |
| L1PA2_dup1750 | Multi | Yes | PEP: 0210, | Redundant | LINE | L1PA\|B\|x | chr5: 147095409-147101435 | − | 10 | 6027 | JAKMIP2 | Proximal | 0 | EVE annotated | 720 |
| | | | PE | | | | | | | | | | | | |
| MSTA_dup6844 | Uniquely | Yes | PEP: 0120 | Non | LTR | ERVL- | chr5: 147758228-147758609 | − | 72 | 382 | FBXO38 | Distal | 4890 | Not documented | 1 |
| | | | | redundant | | MaLR | | | | | | | | | |
| L1HS_dup591 | Multi | Yes | PEP: 0324, | Redundant | LINE | L1PA\|B\|x | chr5: 151456430-151462430 | + | 1.8 | 6001 | GLRA1 | Distal | 152028 | EVE annotated | 2 |
| | | | PE | | | | | | | | | | | | |
| L1HS_dup593 | Multi | Yes | PEP: 0324, | Redundant | LINE | L1PA\|B\|x | chr5: 152266015-152272033 | − | 2.3 | 6019 | NMUR2 | Distal | 453087 | EVE annotated | 3 |
| | | | PE | | | | | | | | | | | | |
| LTR5_Hs_dup206 | Multi | Yes | PEP: 0261 | Redundant | LTR | ERVK | chr5: 156084717-156085684 | − | 4.1 | 968 | SGCD | Proximal | 0 | EVE annotated | 4 |
| L1PA7_dup3988 | Multi | Yes | PEP: 0019, | Redundant | LINE | L1PA\|B\|x | chr5: 15870805-15876520 | + | 39 | 5716 | FBXL7 | Proximal | 0 | EVE annotated | 5 |
| | | | PE | | | | | | | | | | | | |
| L1HS_dup510 | Multi | Yes | PEP: 0324, | Redundant | LINE | L1PA\|B\|x | chr5: 15906625-15912659 | − | 1.4 | 6035 | FBXL7 | Proximal | 0 | EVE annotated | 6 |
| | | | PE | | | | | | | | | | | | |
| L1HS_dup605 | Multi | Yes | PEP: 0324, | Redundant | LINE | L1PA\|B\|x | chr5: 172829800-172835831 | − | 1.8 | 6032 | STC2 | Distal | 73295 | EVE annotated | 7 |
| | | | PE | | | | | | | | | | | | |
| L1HS_dup606 | Multi | Yes | PEP: 0324, | Redundant | LINE | L1PA\|B\|x | chr5: 177199247-177205275 | + | 1.4 | 6029 | FAM153A | Proximal | 0 | EVE annotated | 8 |
| | | | PE | | | | | | | | | | | | |
| L2a_dup63112 | Multi/ | Yes | PEP: 0009 | Non | LINE | L2 | chr5: 180690761-180690883 | + | 150 | 123 | TRIM52 | Distal | 2643 | Not documented | 9 |
| | Uniquely | | | redundant | | | | | | | | | | | |
| L1HS_dup499 | Multi | Yes | PEP: 0210, | Redundant | LINE | L1PA\|B\|x | chr5: 1968733-1971595 | + | 1.4 | 2863 | IRX4 | Distal | 81384 | EVE annotated | 730 |
| | | | PE | | | | | | | | | | | | |
| AluSx3_dup8132 | Multi | Yes | PEP: 0241 | Redundant | SINE | Alu | chr5: 36623676-36623992 | + | 50 | 317 | SLC1A3 | Proximal | 0 | Not documented | 1 |
| L1PA11_dup1278 | Uniquely | Yes | PEP: 0183, | Redundant | LINE | L1PA\|B\|x | chr5: 37102590-37104416 | − | 43 | 1827 | C5orf42 | Proximal | 1915 | Not documented | 2 |
| | | | PE | | | | | | | | | | | | |
| L1PA15-16_dup412 | Uniquely | Yes | PEP: 0303 | Redundant | LINE | L1PA\|B\|x | chr5: 38893346-38895459 | + | 67 | 2114 | OSMR | Proximal | 0 | Not documented | 3 |
| L2c_dup45680 | Multi | Yes | PEP: 0312 | Non | LINE | L2 | chr5: 41919101-41919322 | + | 170 | 222 | C5orf51 | Proximal | 0 | Not documented | 4 |
| | | | | redundant | | | | | | | | | | | |
| L1MB3_dup4843 | Uniquely | Yes | PEP: 0139 | Non | LINE | Other L1 | chr5: 42963151-42964083 | + | 79 | 933 | ANXA2R | Distal | 75253 | Not documented | 5 |
| | | | | redundant | | | | | | | | | | | |
| MSTB_dup2533 | Multi | Yes | PEP: 0018, | Non | LTR | ERVL- | chr5: 43575976-43576125 | + | 80 | 150 | PAIP1 | Distal | 18117 | Not documented | 6 |
| | | | PE | redundant | | MaLR | | | | | | | | | |
| MER39B_dup334 | Multi | Yes | PEP: 0105, | Non | LTR | ERV1 | chr5: 43583316-43583930 | − | 63 | 615 | NNT | Distal | 18865 | Not documented | 7 |
| | | | PE | redundant | | | | | | | | | | | |
| SVA_A_dup65 | Multi | Yes | PEP: 0200 | Redundant | Other | SVA | chr5: 63521560-63522505 | + | 30 | 946 | RNF180 | Proximal | 0 | Not documented | 8 |
| L1PA7_dup4230 | Uniquely | Yes | PEP: 0113 | Redundant | LINE | L1PA\|B\|x | chr5: 63538625-63545092 | + | 47 | 6468 | RNF180 | Proximal | 0 | EVE annotated | 9 |
| AluSz_dup26682 | Multi | Yes | PEP: 0008 | Redundant | SINE | Alu | chr5: 70953262-70953553 | − | 55 | 292 | MCCC2 | Proximal | 0 | Not documented | 740 |
| L1PB1_dup4274 | Multi | Yes | PEP: 0162, | Redundant | LINE | L1PA\|B\|x | chr5: 70979388-70985875 | + | 50 | 6488 | MCCC2 | Distal | 24858 | EVE annotated | 1 |
| | | | PE | | | | | | | | | | | | |
| AluSz_dup26698 | Multi | Yes | PEP: 0338 | Redundant | SINE | Alu | chr5: 71656499-71656813 | − | 58 | 315 | PTCD2 | Proximal | 448 | Not documented | 2 |
| L1HS_dup501 | Multi | Yes | PEP: 0145, | Redundant | LINE | L1PA\|B\|x | chr5: 7603929-7606244 | − | 1.4 | 2316 | ADCY2 | Proximal | 0 | EVE annotated | 3 |
| | | | PE | | | | | | | | | | | | |
| L1HS_dup552 | Multi | Yes | PEP: 0324, | Redundant | LINE | L1PA\|B\|x | chr5: 80911910-80917937 | + | 3.2 | 6028 | SSBP2 | Proximal | 0 | EVE annotated | 4 |
| | | | PE | | | | | | | | | | | | |
| AluSq_dup6064 | Multi | Yes | PEP: 0008 | Redundant | SINE | Alu | chr5: 98251615-98251915 | + | 66 | 301 | CHD1 | Proximal | 0 | Not documented | 5 |
| AluSp_dup14346 | Multi | Yes | PEP: 0319 | Redundant | SINE | Alu | chr5: 98251971-98252274 | + | 46 | 304 | CHD1 | Proximal | 0 | Not documented | 6 |
| SVA_D_dup528 | Multi | Yes | PEP: 0075, | Redundant | Other | SVA | chr6: 108274579-108275684 | + | 25 | 1106 | SEC63 | Proximal | 0 | Not documented | 7 |
| | | | PE | | | | | | | | | | | | |
| Tigger2_dup1140 | Uniquely | Yes | PEP: 0060 | Non | DNA | DNA | chr6: 109735944-109737403 | − | 52 | 1460 | PPIL6 | Proximal | 0 | Not documented | 8 |
| | | | | redundant | | | | | | | | | | | |
| Tigger1_dup5081 | Uniquely | Yes | PEP: 0179 | Non | DNA | DNA | chr6: 111725155-111725802 | + | 48 | 648 | REV3L | Proximal | 0 | Not documented | 9 |
| | | | | redundant | | | | | | | | | | | |
| L1MEc_dup7408 | Uniquely | Yes | PEP: 0098 | Non | LINE | Other L1 | chr6: 112424560-112426778 | + | 98 | 2219 | FAM229B | Proximal | 568 | Not documented | 750 |
| | | | | redundant | | | | | | | | | | | |
| L2b_dup38157 | Uniquely | Yes | PEP: 0337 | Non | LINE | L2 | chr6: 118787923-118788635 | − | 150 | 713 | CEP85L | Proximal | 0 | Not documented | 1 |
| | | | | redundant | | | | | | | | | | | |
| L1PB1_dup5495 | Uniquely | Yes | PEP: 0253 | Non | LINE | L1PA\|B\|x | chr6: 121553857-121556315 | − | 45 | 2459 | TBC1D32 | Proximal | 0 | Not documented | 2 |
| | | | | redundant | | | | | | | | | | | |
| L1HS_dup689 | Multi | Yes | PEP: 0324, | Redundant | LINE | L1PA\|B\|x | chr6: 129319531-129325561 | + | 1.8 | 6031 | LAMA2 | Proximal | 0 | EVE annotated | 3 |
| | | | PE | | | | | | | | | | | | |
| L1PB1_dup5540 | Uniquely | Yes | PEP: 0153, | Redundant | LINE | L1PA\|B\|x | chr6: 132670635-132676547 | − | 48 | 5913 | MOXD1 | Proximal | 0 | EVE annotated | 4 |
| | | | PE | | | | | | | | | | | | |
| L1HS_dup690 | Multi | Yes | PEP: 0324, | Redundant | LINE | L1PA\|B\|x | chr6: 133341856-133347885 | − | 1.8 | 6030 | RPS12 | Distal | 203154 | EVE annotated | 5 |
| | | | PE | | | | | | | | | | | | |
| L2a_dup70960 | Uniquely | Yes | PEP: 0326 | Non | LINE | L2 | chr6: 139240168-139241068 | + | 120 | 901 | REPS1 | Proximal | | Not documented | 6 |
| | | | | redundant | | | | | | | | | | | |
| L1PA11_dup1729 | Uniquely | Yes | PEP: 0022 | Non | LINE | L1PA\|B\|x | chr6: 146192082-146194338 | + | 43 | 2257 | SHPRH | Proximal | 0 | Not documented | 7 |
| | | | | redundant | | | | | | | | | | | |
| L1PA7_dup5716 | Uniquely | Yes | PEP: 0025, | Redundant | LINE | L1PA\|B\|x | chr6: 153431780-153438223 | + | 37 | 6444 | RGS17 | Proximal | 0 | Not documented | 8 |
| | | | PE | | | | | | | | | | | | |
| L1PREC2_dup3231 | Uniquely | Yes | PEP: 0276 | Redundant | LINE | L1PA\|B\|x | chr6: 159936245-159941247 | − | 41 | 5003 | SOD2 | Distal | 148843 | Not documented | 9 |
| SVA_D_dup547 | Multi | Yes | PEP: 0075, | Redundant | Other | SVA | chr6: 160081484-160082279 | + | 15 | 796 | SOD2 | Distal | 7811 | Not documented | 760 |
| | | | PE | | | | | | | | | | | | |
| L1PA5_dup4797 | Multi/ | Yes | PEP: 0324, | Redundant | LINE | L1PA\|B\|x | chr6: 160091647-160097787 | + | 28 | 6141 | SOD2 | Proximal | 0 | EVE annotated | 1 |
| | Uniquely | | PE | | | | | | | | | | | | |
| L2c_dup59892 | Multi | Yes | PEP: 0289 | Redundant | LINE | L2 | chr6: 164002612-164002775 | − | 140 | 164 | QKI | Distal | 2985 | Not documented | 2 |
| MLT1B_dup7324 | Multi/ | Yes | PEP: 0157 | Non | LTR | ERVL- | chr6: 165218359-165218763 | − | 89 | 405 | C6orf118 | Distal | 474391 | Not documented | 3 |
| | Uniquely | | | redundant | | MaLR | | | | | | | | | |
| L1HS_dup616 | Multi | Yes | PEP: 0324, | Redundant | LINE | L1PA\|B\|x | chr6: 24811907-24817934 | − | 1.4 | 6028 | FAM65B | Proximal | 0 | EVE annotated | 4 |
| | | | PE | | | | | | | | | | | | |
| L1ME4a_dup15806 | Multi | Yes | PEP: 0286 | Non | LINE | Other L1 | chr6: 24976284-24976602 | + | 150 | 319 | FAM65B | Proximal | 0 | Not documented | 5 |
| | | | | redundant | | | | | | | | | | | |
| LTR48B_dup407 | Multi | Yes | PEP: 0239 | Non | LTR | ERV1 | chr6: 27106003-27106594 | − | 110 | 592 | HIST1H2BK | Proximal | 0 | Not documented | 6 |
| | | | | redundant | | | | | | | | | | | |
| FRAM_dup2736 | Multi | Yes | PEP: 0272 | Redundant | SINE | Alu | chr6: 28090841-28091010 | + | 78 | 170 | ZSCAN16 | Proximal | 1329 | Not documented | 7 |
| SVA_C_dup103 | Multi | Yes | PEP: 0233, | Redundant | Other | SVA | chr6: 31034380-31035833 | − | 17 | 1454 | MUC22 | Distal | 31202 | Not documented | 8 |
| | | | PE | | | | | | | | | | | | |
| L1PA4_dup4558 | Multi | Yes | PEP: 0053, | Redundant | LINE | L1PA\|B\|x | chr6: 56447400-56451384 | − | 15 | 3985 | DST | Proximal | 0 | EVE annotated | 9 |
| | | | PE | | | | | | | | | | | | |
| L1PA3_dup4098 | Multi/ | Yes | PEP: 0344, | Redundant | LINE | L1PA\|B\|x | chr6: 56644523-56650666 | − | 19 | 6144 | DST | Proximal | 0 | EVE annotated | 770 |
| | Uniquely | | PE | | | | | | | | | | | | |
| SVA_D_dup510 | Multi | Yes | PEP: 0284, | Redundant | Other | SVA | chr6: 56688784-56690317 | + | 13 | 1534 | DST | Proximal | 0 | Not documented | 1 |
| | | | PE | | | | | | | | | | | | |
| L1PA7_dup5219 | Multi/ | Yes | PEP: 0033, | Redundant | LINE | L1PA\|B\|x | chr6: 72065065-72071490 | − | 39 | 6426 | OGFRL1 | Distal | 46413 | EVE annotated | 2 |
| | Uniquely | | PE | | | | | | | | | | | | |
| L1HS_dup643 | Multi | Yes | PEP: 0324, | Redundant | LINE | L1PA\|B\|x | chr6: 73698378-73704409 | − | 2.3 | 6032 | KCNQ5 | Proximal | 0 | EVE annotated | 3 |
| | | | PE | | | | | | | | | | | | |
| HERVK-int_dup100 | Multi | Yes | PEP: 0200 | Redundant | LTR | ERVK | chr6: 78427622-78435123 | − | 1.8 | 7502 | MEI4 | Proximal | 0 | EVE annotated | 4 |
| L1HS_dup654 | Multi | Yes | PEP: 0324, | Redundant | LINE | L1PA\|B\|x | chr6: 86708765-86714791 | − | 1.4 | 6027 | SYNCRIP | Distal | 355256 | EVE annotated | 5 |
| | | | PE | | | | | | | | | | | | |
| L1PA7_dup5409 | Uniquely | Yes | PEP: 0264 | Redundant | LINE | L1PA\|B\|x | chr6: 96919955-96925335 | − | 43 | 5381 | UFL1 | Distal | 44137 | EVE annotated | 6 |
| L1PA6_dup2525 | Uniquely | Yes | PEP: 0113 | Redundant | LINE | L1PA\|B\|x | chr6: 96942251-96948320 | − | 34 | 6070 | UFL1 | Distal | 21152 | EVE annotated | 7 |
| SVA_C_dup125 | Multi | Yes | PEP: 0255, | Redundant | Other | SVA | chr7: 100735044-100736635 | + | 14 | 1592 | TRIM56 | Proximal | 28 | Not documented | 8 |
| | | | PE | | | | | | | | | | | | |
| L1MA3_dup4175 | Uniquely | Yes | PEP: 0342, | Redundant | LINE | Other L1 | chr7: 102827203-102833752 | + | 51 | 6550 | NAPEPLD | Distal | 37197 | Not documented | 9 |
| | | | PE | | | | | | | | | | | | |
| LTR12C_dup1359 | Uniquely | Yes | PEP: 0021 | Redundant | LTR | ERV1 | chr7: 102914527-102915934 | − | 46 | 1408 | PMPCB | Distal | 21936 | Not documented | 780 |
| L1PA14_dup1475 | Uniquely | Yes | PEP: 0128 | Redundant | LINE | L1PA\|B\|x | chr7: 105867820-105869713 | − | 40 | 1894 | NAMPT | Distal | 19019 | Not documented | 1 |
| L1P1_dup1397 | Multi/ | Yes | PEP: 0053, | Redundant | LINE | L1PA\|B\|x | chr7: 105885063-105887369 | − | 4.5 | 2307 | NAMPT | Proximal | 1363 | EVE annotated | 2 |
| | Uniquely | | PE | | | | | | | | | | | | |
| L1PA5_dup5177 | Uniquely | Yes | PEP: 0113, | Redundant | LINE | L1PA\|B\|x | chr7: 106733958-106740073 | − | 31 | 6116 | PRKAR2B | Proximal | 0 | EVE annotated | 3 |
| | | | PE | | | | | | | | | | | | |
| L1P3_dup1516 | Multi | Yes | PEP: 0053 | Redundant | LINE | L1PA\|B\|x | chr7: 107854231-107855656 | − | 40 | 1426 | NRCAM | Proximal | 0 | Not documented | 4 |
| L1P3_dup1517 | Multi/ | Yes | PEP: 0228 | Non | LINE | L1PA\|B\|x | chr7: 107855959-107858840 | − | 42 | 2882 | NRCAM | Proximal | 0 | Not documented | 5 |
| | Uniquely | | | redundant | | | | | | | | | | | |
| L1PA6_dup2928 | Multi/ | Yes | PEP: 0145 | Redundant | LINE | L1PA\|B\|x | chr7: 107913193-107919341 | − | 36 | 6149 | NRCAM | Proximal | 0 | EVE annotated | 6 |
| | Uniquely | | | | | | | | | | | | | | |
| L1PA5_dup5180 | Multi/ | Yes | PEP: 0324, | Redundant | LINE | L1PA\|B\|x | chr7: 108028014-108034176 | − | 28 | 6163 | NRCAM | Proximal | 0 | EVE annotated | 7 |
| | Uniquely | | PE | | | | | | | | | | | | |
| L1HS_dup765 | Multi | Yes | PEP: 0231 | Redundant | LINE | L1PA\|B\|x | chr7: 109547573-109548698 | + | 1.4 | 1126 | IMMP2L | Distal | 754413 | EVE annotated | 8 |
| L1PA7_dup6230 | Uniquely | Yes | PEP: 0303 | Redundant | LINE | L1PA\|B\|x | chr7: 110383534-110389965 | + | 42 | 6432 | IMMP2L | Proximal | 0 | EVE annotated | 9 |
| L1HS_dup713 | Multi | Yes | PEP: 0210, | Redundant | LINE | L1PA\|B\|x | chr7: 110883572-110889602 | − | 1.8 | 6031 | IMMP2L | Proximal | 0 | EVE annotated | 790 |
| | | | PE | | | | | | | | | | | | |
| L1MA7_dup3664 | Uniquely | Yes | PEP: 0307, | Non | LINE | Other L1 | chr7: 110970744-110971756 | + | 72 | 1013 | IMMP2L | Proximal | 0 | Not documented | 1 |
| | | | PE | redundant | | | | | | | | | | | |
| L1HS_dup770 | Multi | Yes | PEP: 0324, | Redundant | LINE | L1PA\|B\|x | chr7: 113416178-113422207 | − | 1.8 | 6030 | PPP1R3A | Distal | 94626 | EVE annotated | 2 |
| | | | PE | | | | | | | | | | | | |
| Charlie1_dup899 | Multi/ | Yes | PEP: 0057 | Non | DNA | DNA | chr7: 119993734-119994655 | + | 97 | 922 | KCND2 | Proximal | 0 | Not documented | 3 |
| | Uniquely | | | redundant | | | | | | | | | | | |
| LTR62_dup96 | Multi | Yes | PEP: 0297 | Non | LTR | ERVL | chr7: 1201954-1202212 | + | 100 | 259 | ZFAND2A | Proximal | 1560 | Not documented | 4 |
| | | | | redundant | | | | | | | | | | | |
| L2a_dup78552 | Multi/ | Yes | PEP: 0035 | Non | LINE | L2 | chr7: 121529616-121530738 | + | 130 | 1123 | PTPRZ1 | Proximal | 0 | Not documented | 5 |
| | Uniquely | | | redundant | | | | | | | | | | | |
| L1PREC2_dup3595 | Uniquely | Yes | PEP: 0052 | Redundant | LINE | L1PA\|B\|x | chr7: 121746353-121747943 | + | 45 | 1591 | AASS | Proximal | 0 | Not documented | 6 |
| L1PA3_dup4963 | Uniquely | Yes | PEP: 0303 | Redundant | LINE | L1PA\|B\|x | chr7: 123085245-123091266 | − | 13 | 6022 | IQUB | Proximal | 1189 | EVE annotated | 7 |
| L1PA17_dup2146 | Uniquely | Yes | PEP: 0279 | Non | LINE | L1PA\|B\|x | chr7: 123802975-123808145 | + | 80 | 5171 | TMEM229A | Distal | 129453 | Not documented | 8 |
| | | | | redundant | | | | | | | | | | | |
| SVA_B_dup198 | Multi | Yes | PEP: 0026, | Redundant | Other | SVA | chr7: 128102917-128104338 | + | 21 | 1422 | HILPDA | Distal | 4446 | Not documented | 9 |
| | | | PE | | | | | | | | | | | | |
| SVA_D_dup608 | Multi | Yes | PEP: 0075, | Redundant | Other | SVA | chr7: 130276379-130277290 | + | 27 | 912 | COPG2 | Proximal | 0 | Not documented | 800 |
| | | | PE | | | | | | | | | | | | |
| L1PA8_dup3780 | Multi/ | Yes | PEP: 0053, | Redundant | LINE | L1PA\|B\|x | chr7: 131000001-131006360 | + | 44 | 6360 | MKLN1 | Proximal | 0 | EVE annotated | 1 |
| | Uniquely | | PE | | | | | | | | | | | | |
| L1PA5_dup5290 | Uniquely | Yes | PEP: 0252, | Redundant | LINE | L1PA\|B\|x | chr7: 134660754-134666906 | + | 32 | 6153 | AGBL3 | Distal | 4354 | EVE annotated | 2 |
| | | | PE | | | | | | | | | | | | |
| L1HS_dup786 | Multi | Yes | PEP: 0324, | Redundant | LINE | L1PA\|B\|x | chr7: 141620482-141626512 | − | 1.4 | 6031 | MGAM | Proximal | 0 | EVE annotated | 3 |
| | | | PE | | | | | | | | | | | | |
| MER57-int_dup524 | Uniquely | Yes | PEP: 0287 | Non | LTR | ERV1 | chr7: 146319-148890 | − | 46 | 2572 | FAM20C | Distal | 44080 | Not documented | 4 |
| | | | | redundant | | | | | | | | | | | |
| AluSz_dup39869 | Multi | Yes | PEP: 0008 | Redundant | SINE | Alu | chr7: 149783054-149783359 | − | 50 | 306 | ACTR3C | Distal | 157647 | Not documented | 5 |
| L1ME2_dup5972 | Uniquely | Yes | PEP: 0142 | Redundant | LINE | Other L1 | chr7: 156599606-156601503 | − | 110 | 1898 | LMBR1 | Proximal | 0 | Not documented | 6 |
| L1HS_dup725 | Multi | Yes | PEP: 0324, | Redundant | LINE | L1PA\|B\|x | chr7: 25081480-25087510 | − | 2.7 | 6031 | OSBPL3 | Distal | 60228 | EVE annotated | 7 |
| | | | PE | | | | | | | | | | | | |
| SVA_E_dup109 | Multi/ | Yes | PEP: 0075, | Redundant | Other | SVA | chr7: 2573126-2574918 | − | 13 | 1793 | BRAT1 | Distal | 2594 | Not documented | 8 |
| | Uniquely | | PE | | | | | | | | | | | | |
| SVA_A_dup94 | Multi/ | Yes | PEP: 0226, | Redundant | Other | SVA | chr7: 26215480-26217248 | − | 28 | 1769 | NFE2L3 | Proximal | 0 | Not documented | 9 |
| | Uniquely | | PE | | | | | | | | | | | | |
| AluJr_dup27455 | Multi/ | Yes | PEP: 0188 | Non | SINE | Alu | chr7: 28860225-28860514 | − | 78 | 290 | CREB5 | Proximal | 0 | Not documented | 810 |
| | Uniquely | | | redundant | | | | | | | | | | | |
| L1HS_dup727 | Multi | Yes | PEP: 0324, | Redundant | LINE | L1PA\|B\|x | chr7: 30478859-30484890 | + | 1.4 | 6032 | NOD1 | Proximal | 0 | EVE annotated | 1 |
| | | | PE | | | | | | | | | | | | |
| L1PA5_dup4915 | Uniquely | Yes | PEP: 0308, | Redundant | LINE | L1PA\|B\|x | chr7: 30577130-30583250 | + | 30 | 6121 | GGCT | Proximal | 0 | EVE annotated | 2 |
| | | | PE | | | | | | | | | | | | |
| L1HS_dup729 | Multi | Yes | PEP: 0210, | Redundant | LINE | L1PA\|B\|x | chr7: 32390207-32393113 | − | 1.4 | 2907 | PDE1C | Distal | 51267 | EVE annotated | 3 |
| | | | PE | | | | | | | | | | | | |
| SVA_B_dup189 | Multi | Yes | PEP: 0284, | Redundant | Other | SVA | chr7: 32792761-32794408 | + | 18 | 1648 | AVL9 | Proximal | 0 | Not documented | 4 |
| | | | PE | | | | | | | | | | | | |
| L1PA3_dup4695 | Multi | Yes | PEP: 0231 | Redundant | LINE | L1PA\|B\|x | chr7: 40021612-40022891 | − | 13 | 1280 | CDK13 | Proximal | 0 | Not documented | 5 |
| HERVK-int_dup103 | Multi | Yes | PEP: 0200 | Redundant | LTR | ERVK | chr7: 4631529-4639063 | − | 2.7 | 7535 | FOXK1 | Distal | 44326 | EVE annotated | 6 |
| L1HS_dup735 | Multi | Yes | PEP: 0210, | Redundant | LINE | L1PA\|B\|x | chr7: 49719865-49725896 | − | 1.4 | 6032 | VWC2 | Distal | 87362 | EVE annotated | 7 |
| | | | PE | | | | | | | | | | | | |
| L1MA10_dup2034 | Uniquely | Yes | PEP: 0364 | Redundant | LINE | Other L1 | chr7: 54482510-54484317 | + | 82 | 1808 | VSTM2A | Distal | 125702 | Not documented | 8 |
| MER33_dup3989 | Multi/ | Yes | PEP: 0369 | Non | DNA | DNA | chr7: 55113502-55113815 | + | 89 | 314 | EGFR | Proximal | 0 | Not documented | 9 |
| | Uniquely | | | redundant | | | | | | | | | | | |
| AluSg4_dup2844 | Multi | Yes | PEP: 0197 | Redundant | SINE | Alu | chr7: 55125807-55126102 | + | 40 | 296 | EGFR | Proximal | 0 | Not documented | 820 |
| L1MB3_dup6728 | Uniquely | Yes | PEP: 0296 | Non | LINE | Other L1 | chr7: 55215302-55215790 | − | 80 | 489 | EGFR | Proximal | 0 | Not documented | 1 |
| | | | | redundant | | | | | | | | | | | |
| AluSx1_dup39801 | Multi | Yes | PEP: 0074 | Redundant | SINE | Alu | chr7: 56155728-56156038 | − | 51 | 311 | PHKG1 | Proximal | 0 | Not documented | 2 |
| ALR/Alpha_dup299 | Multi | Yes | PEP: 0137, | Redundant | Other | Other | chr7: 57948163-57981351 | − | 72 | 33189 | ZNF716 | Distal | 414912 | Not documented | 3 |
| | | | PE | | | Repeats | | | | | | | | | |
| HERVH-int_dup2890 | Multi/ | Yes | PEP: 0305, | Redundant | LTR | ERV1 | chr7: 64139928-64144981 | + | 130 | 5054 | ZNF107 | Proximal | 0 | EVE annotated | 4 |
| | Uniquely | | PE | | | | | | | | | | | | |
| AluSz_dup36207 | Multi | Yes | PEP: 0092 | Redundant | SINE | Alu | chr7: 64436539-64436834 | + | 58 | 296 | ZNF117 | Proximal | 0 | Not documented | 5 |
| HERV3-int_dup100 | Multi/ | Yes | PEP: 0306, | Redundant | LTR | ERV1 | chr7: 64451294-64459721 | − | 67 | 8428 | ERV3-1 | Proximal | 0 | EVE annotated | 6 |
| | Uniquely | | PE | | | | | | | | | | | | |
| L1HS_dup744 | Multi/ | Yes | PEP: 0053, | Redundant | LINE | L1PA\|B\|x | chr7: 65751841-65757871 | − | 2.3 | 6031 | TPST1 | Proximal | 0 | EVE annotated | 7 |
| | Uniquely | | PE | | | | | | | | | | | | |
| L1M1_dup3888 | Multi | Yes | PEP: 0174 | Redundant | LINE | Other L1 | chr7: 65801311-65802286 | + | 110 | 976 | TPST1 | Proximal | 0 | Not documented | 8 |
| SVA_E_dup117 | Multi | Yes | PEP: 0075, | Redundant | Other | SVA | chr7: 65857742-65859109 | − | 35 | 1368 | TPST1 | Proximal | 0 | Not documented | 9 |
| | | | PE | | | | | | | | | | | | |
| MER58A_dup5583 | Uniquely | Yes | PEP: 0039 | Non | DNA | DNA | chr7: 69494714-69494874 | − | 120 | 161 | AUTS2 | Proximal | 0 | Not documented | 830 |
| | | | | redundant | | | | | | | | | | | |
| L1HS_dup745 | Multi | Yes | PEP: 0324, | Redundant | LINE | L1PA\|B\|x | chr7: 69662315-69668343 | + | 1.4 | 6029 | AUTS2 | Proximal | 0 | EVE annotated | 1 |
| | | | PE | | | | | | | | | | | | |
| MIRb_dup91306 | Uniquely | Yes | PEP: 0355 | Non | SINE | MIR | chr7: 7397959-7398195 | + | 120 | 237 | COL28A1 | Proximal | 0 | Not documented | 2 |
| | | | | redundant | | | | | | | | | | | |
| AluSx1_dup41259 | Multi | Yes | PEP: 0338 | Redundant | SINE | Alu | chr7: 74168749-74169041 | + | 51 | 293 | GTF21 | Proximal | 0 | Not documented | 3 |
| L1PA7_dup5783 | Multi/ | Yes | PEP: 0101, | Redundant | LINE | L1PA\|B\|x | chr7: 7427661-7434025 | − | 42 | 6365 | COL28A1 | Proximal | 0 | EVE annotated | 4 |
| | Uniquely | | PE | | | | | | | | | | | | |
| AluSx1_dup41372 | Multi | Yes | PEP: 0338 | Redundant | SINE | Alu | chr7: 74607086-74607378 | − | 51 | 293 | GTF2IRD2B | Distal | 41464 | Not documented | 5 |
| L1HS_dup714 | Multi | Yes | PEP: 0324, | Redundant | LINE | L1PA\|B\|x | chr7: 7504724-7510751 | + | 2.7 | 6028 | COL28A1 | Proximal | 0 | EVE annotated | 6 |
| | | | PE | | | | | | | | | | | | |
| L2c_dup62900 | Multi | Yes | PEP: 0199 | Non | LINE | L2 | chr7: 77313355-77313910 | + | 140 | 55€ | RSBN1L | Distal | 11851 | Not documented | 7 |
| | | | | redundant | | | | | | | | | | | |
| L1PA7_dup6009 | Uniquely | Yes | PEP: 0278 | Redundant | LINE | L1PA\|B\|x | chr7: 77898110-77904716 | − | 40 | 6607 | MAGI2 | Proximal | 0 | Not documented | 8 |
| L1PA5_dup5048 | Multi | Yes | PEP: 0145 | Redundant | LINE | L1PA\|B\|x | chr7: 78484584-78490675 | − | 32 | 6092 | MAGI2 | Proximal | 0 | EVE annotated | 9 |
| L1PA2_dup2210 | Multi | Yes | PEP: 0324, | Redundant | LINE | L1PA\|B\|x | chr7: 78859111-78865127 | − | 9.5 | 6017 | MAGI2 | Proximal | 0 | EVE annotated | 840 |
| | | | PE | | | | | | | | | | | | |
| L1PA17_dup1942 | Uniquely | Yes | PEP: 0370 | Non | LINE | L1PA\|B\|x | chr7: 8074858-8075524 | + | 160 | 667 | GLCCI1 | Proximal | 0 | Not documented | 1 |
| | | | | redundant | | | | | | | | | | | |
| L1PA7_dup5786 | Uniquely | Yes | PEP: 0303 | Redundant | LINE | L1PA\|B\|x | chr7: 8135955-8141898 | + | 45 | 5944 | GLCCI1 | Distal | 2054 | Not documented | 2 |
| HAL1_dup12404 | Uniquely | Yes | PEP: 0095 | Non | LINE | Other L1 | chr7: 81921336-81923456 | − | 130 | 2121 | CACNA2D1 | Proximal | 0 | Not documented | 3 |
| | | | | redundant | | | | | | | | | | | |
| L1PA2_dup2078 | Multi | Yes | PEP: 0324, | Redundant | LINE | L1PA\|B\|x | chr7: 83618642-83624658 | − | 13 | 6017 | SEMA3A | Proximal | 0 | EVE annotated | 4 |
| | | | PE | | | | | | | | | | | | |
| AluJb_dup56043 | Multi/ | Yes | PEP: 0314 | Non | SINE | Alu | chr7: 87028509-87028812 | + | 78 | 304 | CROT | Proximal | 0 | Not documented | 5 |
| | Uniquely | | | redundant | | | | | | | | | | | |
| L1PB4_dup3202 | Uniquely | Yes | PEP: 0159 | Non | LINE | L1PA\|B\|x | chr7: 88220859-88226583 | + | 89 | 5725 | ZNF804B | Distal | 162100 | Not documented | 6 |
| | | | | redundant | | | | | | | | | | | |
| L1PA7_dup6101 | Uniquely | Yes | PEP: 0303 | Redundant | LINE | L1PA\|B\|x | chr7: 90359190-90365596 | − | 45 | 6407 | CDK14 | Proximal | 0 | EVE annotated | 7 |
| L1PA4_dup5112 | Multi | Yes | PEP: 0324, | Redundant | LINE | L1PA\|B\|x | chr7: 90435806-90441945 | + | 25 | 6140 | CDK14 | Proximal | 0 | EVE annotated | |
| | | | PE | | | | | | | | | | | | |
| L1PA8_dup3441 | Uniquely | Yes | PEP: 0303 | Redundant | LINE | L1PA\|B\|x | chr7: 90700551-90706986 | + | 50 | 6436 | CDK14 | Proximal | 0 | EVE annotated | 9 |
| L1PA3_dup4854 | Uniquely | Yes | PEP: 0113, | Redundant | LINE | L1PA\|B\|x | chr7: 91813071-91819192 | − | 16 | 6122 | LRRD1 | Distal | 4227 | EVE annotated | 850 |
| | | | PE | | | | | | | | | | | | |
| L1PA13_dup3928 | Uniquely | Yes | PEP: 0352, | Redundant | LINE | L1PA\|B\|x | chr7: 95242427-95245758 | + | 59 | 3332 | PDK4 | Distal | 16625 | Not documented | 1 |
| | | | PE | | | | | | | | | | | | |
| L1MA2_dup2984 | Uniquely | Yes | PEP: 0211 | Redundant | LINE | Other L1 | chr7: 96255762-96260885 | − | 60 | 5124 | SHFM1 | Proximal | 0 | Not documented | 2 |
| L1HS_dup759 | Multi | Yes | PEP: 0324, | Redundant | LINE | L1PA\|B\|x | chr7: 96475963-96481992 | − | 1.4 | 6030 | SHFM1 | Distal | 136761 | EVE annotated | 3 |
| | | | PE | | | | | | | | | | | | |
| LTR12C_dup1354 | Multi | Yes | PEP: 0010 | Redundant | LTR | ERV1 | chr7: 97565059-97566447 | + | 32 | 1389 | OCM2 | Distal | 47550 | Not documented | 4 |
| L1PA4_dup5456 | Uniquely | Yes | PEP: 0113 | Redundant | LINE | L1PA\|B\|x | chr7: 99251709-99257824 | − | 24 | 6116 | CYP3A5 | Proximal | 0 | EVE annotated | 5 |
| HERVH-int_dup2926 | Uniquely | Yes | PEP: 0180 | Non | LTR | ERV1 | chr7: 99321692-99325902 | + | 92 | 4211 | CYP3A7 | Proximal | 0 | EVE annotated | 6 |
| | | | | redundant | | | | | | | | | | | |
| L1PA3_dup5574 | Multi | Yes | PEP: 0231, | Redundant | LINE | L1PA\|B\|x | chr8: 122530595-122531667 | − | 10 | 1073 | HAS2 | Distal | 92690 | Not documented | 7 |
| | | | PE | | | | | | | | | | | | |
| L1HS_dup848 | Multi | Yes | PEP: 0324, | Redundant | LINE | L1PA\|B\|x | chr8: 126595131-126601133 | + | 1.4 | 6003 | TRIB1 | Distal | 144485 | EVE annotated | 8 |
| | | | PE | | | | | | | | | | | | |
| L1HS_dup851 | Multi | Yes | PEP: 0324, | Redundant | LINE | L1PA\|B\|x | chr8: 129465236-129471266 | − | 1.4 | 6031 | TMEM75 | Distal | 504646 | EVE annotated | 9 |
| | | | PE | | | | | | | | | | | | |
| L1HS_dup855 | Multi | Yes | PEP: 0324, | Redundant | LINE | L1PA\|B\|x | chr8: 135082987-135089016 | − | 1.8 | 6030 | ZFAT | Distal | 401016 | EVE annotated | 860 |
| | | | PE | | | | | | | | | | | | |
| L1PA5_dup5960 | Uniquely | Yes | PEP: 0113 | Redundant | LINE | L1PA\|B\|x | chr8: 135679867-135684575 | + | 31 | 4709 | ZFAT | Proximal | 0 | EVE annotated | 1 |
| L1HS_dup803 | Multi | Yes | PEP: 0145, | Redundant | LINE | L1PA\|B\|x | chr8: 18454618-18455933 | + | 1.8 | 1316 | PSD3 | Proximal | 0 | Not documented | 2 |
| | | | PE | | | | | | | | | | | | |
| AluSx1_dup45754 | Multi/ | Yes | PEP: 0201 | Redundant | SINE | Alu | chr8: 28048091-28048375 | − | 38 | 285 | ELP3 | Proximal | 0 | Not documented | 3 |
| | Uniquely | | | | | | | | | | | | | | |
| L1MC3_dup6004 | Multi | Yes | PEP: 0084, | Redundant | LINE | Other L1 | chr8: 32898724-32900112 | − | 85 | 1389 | NRG1 | Distal | 276177 | Not documented | 4 |
| | | | PE | | | | | | | | | | | | |
| L1PA5_dup5526 | Uniquely | Yes | PEP: 0303 | Redundant | LINE | L1PA\|B\|x | chr8: 43038792-43044885 | − | 29 | 6094 | HGSNAT | Proximal | 0 | EVE annotated | 5 |
| SAR | Uniquely | Yes | PEP: 0109, | Redundant | Other | Other | chr8: 43092746-43097520 | + | 49 | 4775 | HGSNAT | Distal | 34749 | Not documented | 6 |
| | | | PE | | | Repeats | | | | | | | | | |
| L1HS_dup818 | Multi | Yes | PEP: 0231 | Redundant | LINE | L1PA\|B\|x | chr8: 48244565-48245610 | − | 0.91 | 1046 | SPIDR | Proximal | 0 | Not documented | 7 |
| L1PA2_dup2457 | Multi | Yes | PEP: 0324, | Redundant | LINE | L1PA\|B\|x | chr8: 59827250-59833276 | + | 3.6 | 6027 | TOX | Proximal | 0 | EVE annotated | 8 |
| | | | PE | | | | | | | | | | | | |
| L1PA3_dup5302 | Uniquely | Yes | PEP: 0113, | Redundant | LINE | L1PA\|B\|x | chr8: 63713193-63719375 | + | 20 | 6183 | NKAIN3 | Proximal | 0 | EVE annotated | 9 |
| | | | PE | | | | | | | | | | | | |
| LTR5_Hs_dup281 | Multi | Yes | PEP: 0261 | Redundant | LTR | ERVK | chr8: 7355397-7356364 | − | 4.5 | 968 | DEFB107B | Proximal | 0 | EVE annotated | 870 |
| HERVK-int_dup114 | Multi | Yes | PEP: 0200 | Redundant | LTR | ERVK | chr8: 7356365-7363899 | − | 2.7 | 7535 | DEFB107B | Proximal | 0 | EVE annotated | 1 |
| L1HS_dup795 | Multi | Yes | PEP: 0324, | Redundant | LINE | L1PA\|B\|x | chr8: 73787793-73793823 | − | 1.4 | 6031 | KCNB2 | Proximal | 0 | EVE annotated | 2 |
| | | | PE | | | | | | | | | | | | |
| L1PA6_dup3134 | Multi/ | Yes | PEP: 0219, | Redundant | LINE | L1PA\|B\|x | chr8: 73968585-73974141 | + | 30 | 5557 | SBSPON | Distal | 2635 | Not documented | 3 |
| | Uniquely | | PE | | | | | | | | | | | | |
| L1HS_dup826 | Multi | Yes | PEP: 0231 | Redundant | LINE | L1PA\|B\|x | chr8: 77337395-77338429 | − | 1.4 | 1035 | ZFHX4 | Distal | 255026 | Not documented | 4 |
| HERVK-int_dup117 | Multi | Yes | PEP: 0200 | Redundant | LTR | ERVK | chr8: 8055726-8063197 | + | 70 | 7472 | LRLE1 | Distal | 9396 | EVE annotated | 5 |
| L1ME2_dup6351 | Multi/ | Yes | PEP: 0051 | Redundant | LINE | Other L1 | chr8: 82354600-82355036 | + | 110 | 437 | PMP2 | Proximal | 0 | Not documented | 6 |
| | Uniquely | | | | | | | | | | | | | | |
| Charlie15a_dup1641 | Multi | Yes | PEP: 0259 | Non | DNA | DNA | chr8: 87572503-87572689 | − | 130 | 187 | CPNE3 | Proximal | 0 | Not documented | 7 |
| | | | | redundant | | | | | | | | | | | |
| L1M2_dup4322 | Multi | Yes | PEP: 0076 | Non | LINE | Other L1 | chr8: 90758293-90760495 | + | 100 | 2203 | RIPK2 | Distal | 9481 | Not documented | 8 |
| | | | | redundant | | | | | | | | | | | |
| L1M6_dup3686 | Multi/ | Yes | PEP: 0071 | Non | LINE | Other L1 | chr8: 92081092-92081837 | − | 120 | 746 | OTUD6B | Proximal | 588 | Not documented | 9 |
| | Uniquely | | | redundant | | | | | | | | | | | |
| L1PA3_dup5481 | Uniquely | Yes | PEP: 0303 | Redundant | LINE | L1PA\|B\|x | chr8: 99596326-99602350 | − | 13 | 6025 | STK3 | Proximal | 0 | EVE annotated | 880 |
| L1PA3_dup6018 | Multi | Yes | PEP: 0145, | Redundant | LINE | L1PA\|B\|x | chr9: 106478667-106480752 | − | 13 | 2086 | SMC2 | Distal | 375790 | EVE annotated | 1 |
| | | | PE | | | | | | | | | | | | |
| L1ME4a_dup23477 | Multi | Yes | PEP: 0346 | Non | LINE | Other L1 | chr9: 111702230-111702341 | − | 110 | 112 | FAM206A | Proximal | 0 | Not documented | 2 |
| | | | | redundant | | | | | | | | | | | |
| L1PA4_dup6699 | Uniquely | Yes | PEP: 0349, | Redundant | LINE | L1PA\|B\|x | chr9: 112991699-112998191 | − | 22 | 6493 | TXN | Distal | 7901 | EVE annotated | 3 |
| | | | PE | | | | | | | | | | | | |
| Charlie5_dup1370 | Multi | Yes | PEP: 0265 | Non | DNA | DNA | chr9: 114313982-114316040 | − | 80 | 2059 | PTGR1 | Proximal | 0 | Not documented | 4 |
| | | | | redundant | | | | | | | | | | | |
| FRAM_dup4210 | Multi | Yes | PEP: 0047 | Redundant | SINE | Alu | chr9: 116169844-116170003 | + | 63 | 160 | POLE3 | Proximal | 0 | Not documented | 5 |
| AluY_dup60542 | Multi | Yes | PEP: 0338 | Redundant | SINE | AIL | chr9: 117849969-117850281 | − | 36 | 313 | TNC | Proximal | 0 | Not documented | 6 |
| L1PA6_dup3275 | Uniquely | Yes | PEP: 0113 | Redundant | LINE | L1PA\|B\|x | chr9: 123861305-123866811 | + | 27 | 5507 | CNTRL | Proximal | 0 | EVE annotated | 7 |
| LTR5_Hs_dup327 | Multi | Yes | PEP: 0261 | Redundant | LTR | ERVK | chr9: 124191384-124192351 | + | 5 | 968 | STOM | Distal | 58854 | Not documented | 8 |
| L1PA4_dup6744 | Multi | Yes | PEP: 0324, | Redundant | LINE | L1PA\|B\|x | chr9: 125521300-125524151 | + | 28 | 2852 | OR1L6 | Distal | 8239 | Not documented | 9 |
| | | | PE | | | | | | | | | | | | |
| SVA_D_dup714 | Multi | Yes | PEP: 0075, | Redundant | Other | SVA | chr9: 125629748-125631014 | + | 35 | 1267 | RC3H2 | Proximal | 0 | Not documented | 890 |
| | | | PE | | | | | | | | | | | | |
| L1HS_dup866 | Multi | Yes | PEP: 0231 | Redundant | LINE | L1PA\|B\|x | chr9: 1338002-1338982 | − | 0.91 | 981 | DMRT2 | Distal | 280451 | Not documented | 1 |
| L1HS_dup920 | Multi | Yes | PEP: 0145, | Redundant | LINE | L1PA\|B\|x | chr9: 141078377-141080926 | − | 3.2 | 2550 | CACNA1B | Distal | 59302 | EVE annotated | 2 |
| | | | PE | | | | | | | | | | | | |
| L1PA3_dup5659 | Multi/ | Yes | PEP: 0145, | Redundant | LINE | L1PA\|B\|x | chr9: 4186005-4189898 | − | 16 | 3894 | GLIS3 | Proximal | 0 | EVE annotated | 3 |
| | Uniquely | | PE | | | | | | | | | | | | |
| SVA_F_dup497 | Multi | Yes | PEP: 0075, | Redundant | Other | SVA | chr9: 42128134-42128791 | + | 6.4 | 658 | ANKRD20A2 | Distal | 239513 | Not documented | 4 |
| | | | PE | | | | | | | | | | | | |
| L1PA7_dup7120 | Multi/ | Yes | PEP: 0156, | Redundant | LINE | L1PA\|B\|x | chr9: 4248930-4254064 | − | 44 | 5135 | GLIS3 | Proximal | 0 | EVE annotated | 5 |
| | Uniquely | | PE | | | | | | | | | | | | |
| SST1_dup72 | Multi | Yes | PEP: 0347 | Redundant | Other | Other | chr9: 44989880-44990691 | + | 110 | 812 | FAM27C | Proximal | 0 | Not documented | 6 |
| | | | | | | Repeats | | | | | | | | | |
| L1HS_dup883 | Multi | Yes | PEP: 0324, | Redundant | LINE | L1PA\|B\|x | chr9: 46439644-46445703 | − | 1.8 | 6060 | FAM27D1 | Distal | 48089 | Not documented | 7 |
| | | | PE | | | | | | | | | | | | |
| SVA_F_dup498 | Multi | Yes | PEP: 0075, | Redundant | Other | SVA | chr9: 46578247-46578904 | − | 6.4 | 658 | FAM27D1 | Distal | 186692 | Not documented | 8 |
| | | | PE | | | | | | | | | | | | |
| Tigger1_dup6601 | Uniquely | Yes | PEP: 0293 | Redundant | DNA | DNA | chr9: 4725912-4728263 | − | 50 | 2352 | AK3 | Proximal | 0 | Not documented | 9 |
| L1PA6_dup3365 | Multi | Yes | PEP: 0112 | Redundant | LINE | L1PA\|B\|x | chr9: 66478268-66484289 | − | 44 | 6022 | SPATA31A7 | Distal | 968659 | EVE annotated | 900 |
| ALR/Alpha_dup555 | Multi | Yes | PEP: 0227, | Redundant | Other | Other | chr9: 66767879-66826625 | + | 59 | 58747 | FAM27E3 | Distal | 958320 | Not documented | 1 |
| | | | PE | | | Repeats | | | | | | | | | |
| ALR/Alpha_dup545 | Multi | Yes | PEP: 0148 | Redundant | Other | Other | chr9: 66839600-66853530 | + | 72 | 13931 | FAM27E3 | Distal | 931415 | Not documented | 2 |
| | | | | | | Repeats | | | | | | | | | |
| ALR/Alpha_dup546 | Multi | Yes | PEP: 0336 | Redundant | Other | Other | chr9: 66970956-66992580 | − | 72 | 21625 | FAM27E3 | Distal | 792365 | Not documented | 3 |
| | | | | | | Repeats | | | | | | | | | |
| SST1_dup105 | Multi | Yes | PEP: 0347 | Redundant | Other | Other | chr9: 67793734-67794545 | − | 110 | 812 | FAM27B | Proximal | 0 | Not documented | 4 |
| | | | | | | Repeats | | | | | | | | | |
| L1PA6_dup3370 | Multi | Yes | PEP: 0053, | Redundant | LINE | L1PA\|B\|x | chr9: 68385162-68388929 | + | 44 | 3768 | RP11-195B21.3 | Distal | 397165 | EVE annotated | 5 |
| | | | PE | | | | | | | | | | | | |
| L1MA9_dup8907 | Multi | Yes | PEP: 0160 | Redundant | LINE | Other L1 | chr9: 68701109-68703344 | − | 98 | 2236 | FOXD4L6 | Distal | 496137 | Not documented | 6 |
| ALR/Alpha_dup562 | Multi | Yes | PEP: 0336 | Redundant | Other | Other | chr9: 69689765-69711386 | + | 68 | 21622 | AL445665.1 | Distal | 29408 | Not documented | 7 |
| | | | | | | Repeats | | | | | | | | | |
| ALR/Alpha_dup547 | Multi/ | Yes | PEP: 0037, | Redundant | Other | Other | chr9: 69948086-70000000 | + | 62 | 51915 | FOXD4L5 | Distal | 175708 | Not documented | 8 |
| | Uniquely | | PE | | | Repeats | | | | | | | | | |
| ALR/Alpha_dup563 | Multi | Yes | PEP: 0031, | Redundant | Other | Other | chr9: 70000002-70010538 | + | 58 | 10537 | FOXD4L5 | Distal | 165170 | Not documented | 9 |
| | | | PE | | | Repeats | | | | | | | | | |
| L1PA8_dup4485 | Multi/ | Yes | PEP: 0168, | Redundant | LINE | L1PA\|B\|x | chr9: 72794790-72800921 | − | 48 | 6132 | MAMDC2 | Proximal | 0 | EVE annotated | 910 |
| | Uniquely | | PE | | | | | | | | | | | | |
| L1MB7_dup10948 | Uniquely | Yes | PEP: 0206, | Non | LINE | Other L1 | chr9: 72803820-72806017 | − | 100 | 2198 | MAMDC2 | Proximal | 0 | Not documented | 1 |
| | | | PE | redundant | | | | | | | | | | | |
| L1PA2_dup2760 | Multi/ | Yes | PEP: 0169, | Redundant | LINE | L1PA\|B\|x | chr9: 72813973-72819997 | − | 8.2 | 6025 | MAMDC2 | Proximal | 0 | EVE annotated | 2 |
| | Uniquely | | PE | | | | | | | | | | | | |
| L1PA5_dup6270 | Multi/ | Yes | PEP: 0219, | Redundant | LINE | L1PA\|B\|x | chr9: 72821546-72827671 | − | 32 | 6126 | MAMDC2 | Proximal | 0 | EVE annotated | 3 |
| | Uniquely | | PE | | | | | | | | | | | | |
| L2a_dup91612 | Uniquely | Yes | PEP: 0170 | Redundant | LINE | L2 | chr9: 72836708-72837363 | − | 130 | 656 | MAMDC2 | Proximal | 0 | Not documented | 4 |
| AluSc8_dup10464 | Multi | Yes | PEP: 0196 | Redundant | SINE | Alu | chr9: 78609329-78609637 | − | 32 | 309 | PCSK5 | Proximal | 0 | Not documented | 5 |
| L2c_dup77461 | Multi/ | Yes | PEP: 0223 | Non | LINE | L2 | chr9: 96209512-96209644 | + | 140 | 133 | FAM120AOS | Proximal | 0 | Not documented | 6 |
| | Uniquely | | | redundant | | | | | | | | | | | |
| L1PA5_dup6342 | Multi | Yes | PEP: 0219, | Redundant | LINE | L1PA\|B\|x | chr9: 96979026-96985164 | + | 28 | 6139 | ZNF169 | Distal | 36430 | EVE annotated | 7 |
| | | | PE | | | | | | | | | | | | |
| L1HS_dup904 | Multi | Yes | PEP: 0324, | Redundant | LINE | L1PA\|B\|x | chr9: 98459868-98465900 | + | 1.4 | 6033 | DKFZP434H0512 | Distal | 68706 | EVE annotated | 8 |
| | | | PE | | | | | | | | | | | | |
| L1PA2_dup3131 | Multi | Yes | PEP: 0053, | Redundant | LINE | L1PA\|B\|x | chrX: 108297821-108302024 | + | 9.5 | 4204 | GUCY2F | Distal | 314112 | EVE annotated | 9 |
| | | | PE | | | | | | | | | | | | |
| L1PA8_dup5041 | Multi | Yes | PEP: 0146, | Redundant | LINE | L1PA\|B\|x | chrX: 108447417-108452584 | − | 62 | 5168 | GUCY2F | Distal | 163552 | Not documented | 920 |
| | | | PE | | | | | | | | | | | | |
| L2_dup34098 | Multi | Yes | PEP: 0267 | Non | LINE | L2 | chrX: 108461301-108463314 | − | 170 | 2014 | GUCY2F | Distal | 152822 | Not documented | 1 |
| | | | | redundant | | | | | | | | | | | |
| Harlequin-int_dup331 | Multi | Yes | PEP: 0135 | Redundant | LTR | ERV1 | chrX: 115033232-115040112 | − | 42 | 6881 | RP1-241P17.4 | Distal | 79564 | EVE annotated | 2 |
| L1PA7_dup8551 | Uniquely | Yes | PEP: 0303 | Redundant | LINE | L1PA\|B\|x | chrX: 117072906-117078207 | − | 39 | 5302 | KLHL13 | Proximal | 0 | EVE annotated | 3 |
| L1HS_dup923 | Multi | Yes | PEP: 0324, | Redundant | LINE | L1PA\|B\|x | chrX: 11725369-11731399 | + | 1.4 | 6031 | ARHGAP6 | Distal | 41549 | EVE annotated | 4 |
| | | | PE | | | | | | | | | | | | |
| L1HS_dup1014 | Multi | Yes | PEP: 0324, | Redundant | LINE | L1PA\|B\|x | chrX: 118569432-118575456 | − | 1.4 | 6025 | SLC25A43 | Proximal | 0 | EVE annotated | 5 |
| | | | PE | | | | | | | | | | | | |
| L1HS_dup924 | Multi | Yes | PEP: 0210, | Redundant | LINE | L1PA\|B\|x | chrX: 11953208-11959433 | − | 4.5 | 6226 | MSL3 | Distal | 159339 | EVE annotated | 6 |
| | | | PE | | | | | | | | | | | | |
| L1PB1_dup8747 | Uniquely | Yes | PEP: 0366 | Redundant | LINE | L1PA\|B\|x | chrX: 128384209-128389887 | − | 44 | 5679 | SMARCA1 | Distal | 190594 | Not documented | 7 |
| L1HS_dup1037 | Multi | Yes | PEP: 0324, | Redundant | LINE | L1PA\|B\|x | chrX: 146735253-146741285 | + | 1.8 | 6033 | FMR1 | Distal | 252185 | EVE annotated | 8 |
| | | | PE | | | | | | | | | | | | |
| AluSp_dup25151 | Multi | Yes | PEP: 0345 | Redundant | SINE | Alu | chrX: 38186030-38186327 | + | 25 | 298 | RPGR | Proximal | 0 | Not documented | 9 |
| L1P3_dup1956 | Uniquely | Yes | PEP: 0303 | Redundant | LINE | L1PA\|B\|x | chrX: 49154125-49156946 | − | 45 | 2822 | PPP1R3F | Proximal | 0 | Not documented | 930 |
| L1PA15_dup4778 | Uniquely | Yes | PEP: 0299 | Non | LINE | L1PA\|B\|x | chrX: 56164229-56167209 | + | 70 | 2981 | AL353698.1 | Distal | 62216 | Not documented | 1 |
| | | | | redundant | | | | | | | | | | | |
| L1PA6_dup3590 | Multi | Yes | PEP: 0363, | Redundant | LINE | L1PA\|B\|x | chrX: 56515967-56522017 | − | 39 | 6051 | UBQLN2 | Distal | 68010 | EVE annotated | 2 |
| | | | PE | | | | | | | | | | | | |
| HERVH48-int_dup69 | Multi | Yes | PEP: 0127 | Non | LTR | ERV1 | chrX: 57246896-57250706 | + | 17 | 3811 | FAAH2 | Distal | 62434 | EVE annotated | 3 |
| | | | | redundant | | | | | | | | | | | |
| L1PA13_dup5216 | Uniquely | Yes | PEP: 0117, | Non | LINE | L1PA\|B\|x | chrX: 64933942-64935947 | − | 42 | 2006 | MSN | Proximal | 0 | Not documented | 4 |
| | | | PE | redundant | | | | | | | | | | | |
| L1PA5_dup6871 | Uniquely | Yes | PEP: 0113, | Redundant | LINE | L1PA\|B\|x | chrX: 73253779-73259910 | − | 28 | 6132 | ZCCHC13 | Distal | 264116 | EVE annotated | 5 |
| | | | PE | | | | | | | | | | | | |
| L1PA7_dup8064 | Uniquely | Yes | PEP: 0113, | Redundant | LINE | L1PA\|B\|x | chrX: 73260446-73266814 | − | 43 | 6369 | ZCCHC13 | Distal | 257212 | EVE annotated | 6 |
| | | | PE | | | | | | | | | | | | |
| L1PREC2_dup4604 | Uniquely | Yes | PEP: 0257 | Non | LINE | L1PA\|B\|x | chrX: 74951171-74955557 | − | 88 | 4387 | MAGEE2 | Distal | 47267 | Not documented | 7 |
| | | | | redundant | | | | | | | | | | | |
| L1PA7_dup8084 | Uniquely | Yes | PEP: 0218 | Non | LINE | L1PA\|B\|x | chrX: 75033811-75040159 | + | 50 | 6349 | MAGEE2 | Distal | 28733 | EVE annotated | 8 |
| | | | | redundant | | | | | | | | | | | |
| HERVL18-int_dup353 | Uniquely | Yes | PEP: 0323 | Non | LTR | ERVL | chrX: 76017214-76023243 | + | 67 | 6030 | MAGEE1 | Distal | 365471 | EVE annotated | 9 |
| | | | | redundant | | | | | | | | | | | |
| L1PA14_dup1949 | Uniquely | Yes | PEP: 0317 | Non | LINE | L1PA\|B\|x | chrX: 80979302-80982925 | + | 91 | 3624 | SH3BGRL | Distal | 425257 | Not documented | 940 |
| | | | | redundant | | | | | | | | | | | |
| L1HS_dup979 | Multi | Yes | PEP: 0324, | Redundant | LINE | L1PA\|B\|x | chrX: 81096653-81102683 | + | 2.3 | 6031 | SH3BGRL | Distal | 542608 | EVE annotated | 1 |
| | | | PE | | | | | | | | | | | | |
| L1PA15_dup5004 | Uniquely | Yes | PEP: 0073 | Non | LINE | L1PA\|B\|x | chrX: 82481963-82487154 | − | 99 | 5192 | POU3F4 | Distal | 276116 | Not documented | 2 |
| | | | | redundant | | | | | | | | | | | |
| L1M1_dup4817 | Uniquely | Yes | PEP: 0211 | Redundant | LINE | Other L1 | chrX: 82702381-82707439 | − | 72 | 5059 | POU3F4 | Distal | 55831 | Not documented | 3 |
| L1M1_dup5626 | Uniquely | Yes | PEP: 0192 | Redundant | LINE | Other L1 | chrX: 86802848-86806557 | + | 73 | 3710 | KLHL4 | Proximal | 0 | Not documented | 4 |
| SVA_D_dup775 | Multi | Yes | PEP: 0075, | Redundant | Other | SVA | chrX: 99978130-99979868 | + | 20 | 1739 | SYTL4 | Proximal | 0 | Not documented | 5 |
| | | | PE | | | | | | | | | | | | |
| ALR/Alpha_dup665 | Multi | Yes | PEP: 0336 | Redundant | Other | Other | chrY: 10080737-10093376 | − | 75 | 12640 | FAM197Y1 | Distal | 696045 | Not documented | 6 |
| | | | | | | Repeats | | | | | | | | | |
| L1HS_dup1055 | Multi | Yes | PEP: 0324, | Redundant | LINE | L1PA\|B\|x | chrY: 5474210-5480240 | − | 2.3 | 6031 | PCDH11Y | Proximal | 0 | EVE annotated | 7 |
| | | | PE | | | | | | | | | | | | |
| L1PA3_dup7163 | Multi | Yes | PEP: 0169, | Redundant | LINE | L1PA\|B\|x | chrY: 9969663-9971179 | − | 18 | 1517 | FAM197Y1 | Distal | 584971 | Not documented | 8 |
| | | | PE | | | | | | | | | | | | |
| AluSq2_dup2383 | Uniquely | No | — | — | SINE | Alu | chr1: 100437444-100437756 | − | 39 | 313 | SLC35A3 | Proximal | 0 | Not documented | 9 |
| AluSz_dup4285 | Uniquely | No | — | — | SINE | Alu | chr1: 100437888-100438191 | − | 45 | 304 | SLC35A3 | Proximal | 0 | Not documented | 950 |
| MER11A_dup25 | Uniquely | No | — | — | LTR | ERVK | chr1: 100729324-100730419 | + | 22 | 1096 | RTCA | Proximal | 1345 | Not documented | 1 |
| L1MB2_dup268 | Uniquely | No | — | — | LINE | Other L1 | chr1: 101353797-101354285 | + | 77 | 489 | EXTL2 | Proximal | 0 | Not documented | 2 |
| AluSq2_dup2403 | Multi | No | — | — | SINE | Alu | chr1: 101443294-101443609 | − | 55 | 316 | SLC30A7 | Proximal | 0 | Not documented | 3 |
| MIR3_dup4855 | Multi | No | — | — | SINE | MIR | chr1: 101444005-101444198 | − | 140 | 194 | SLC30A7 | Proximal | 0 | Not documented | 4 |
| THE1D_dup387 | Multi | No | — | — | LTR | ERVL- | chr1: 101455572-101455915 | − | 47 | 344 | DPH5 | Proximal | 0 | Not documented | 5 |
| | | | | | | MaLR | | | | | | | | | |
| LTR6A_dup7 | Multi | No | — | — | LTR | ERV1 | chr1: 102252741-102252770 | + | 15 | 30 | OLFM3 | Distal | 15361 | Not documented | 6 |
| FAM_dup16 | Multi | No | — | — | SINE | Alu | chr1: 10331227-10331376 | − | 58 | 150 | KIF1B | Proximal | 0 | Not documented | 7 |
| Tigger1_dup432 | Uniquely | No | — | — | DNA | DNA | chr1: 103522254-103524702 | − | 68 | 2449 | COL11A1 | Proximal | 0 | Not documented | 8 |
| MIRc_dup288 | Multi | No | — | — | SINE | MIR | chr1: 10358774-10358862 | − | 100 | 89 | KIF1B | Proximal | 0 | Not documented | 9 |
| AluYk11_dup9 | Multi | No | — | — | SINE | Alu | chr1: 10358932-10358993 | + | 37 | 62 | KIF1B | Proximal | 0 | Not documented | 960 |
| AluSx_dup966 | Multi | No | — | — | SINE | Alu | chr1: 10367358-10367667 | − | 53 | 310 | KIF1B | Proximal | 0 | Not documented | 1 |
| L2b_dup406 | Uniquely | No | — | — | LINE | L2 | chr1: 10371571-10372991 | + | 140 | 1421 | KIF1B | Proximal | 0 | Not documented | 2 |
| AluSx_dup981 | Multi | No | — | — | SINE | Alu | chr1: 10440484-10440777 | + | 45 | 294 | KIF1B | Proximal | 0 | Not documented | 3 |
| MIRb_dup609 | Multi | No | — | — | SINE | MIR | chr1: 10491100-10491219 | + | 140 | 120 | APITD1 | Proximal | 0 | Not documented | 4 |
| AluY_dup682 | Multi/ | No | — | — | SINE | Alu | chr1: 10520965-10521264 | − | 29 | 300 | DFFA | Proximal | 0 | Not documented | 5 |
| | Uniquely | | | | | | | | | | | | | | |
| AluSz_dup4434 | Multi | No | — | — | SINE | Alu | chr1: 109474613-109474905 | + | 51 | 293 | CLCC1 | Proximal | 0 | Not documented | 6 |
| MER5A_dup1187 | Multi/ | No | — | — | DNA | DNA | chr1: 109505048-109505116 | − | 99 | 69 | CLCC1 | Proximal | 0 | Not documented | 7 |
| | Uniquely | | | | | | | | | | | | | | |
| AluSz_dup4440 | Uniquely | No | — | — | SINE | Alu | chr1: 109505210-109505508 | + | 65 | 299 | CLCC1 | Proximal | 0 | Not documented | 8 |
| AluY_dup5129 | Multi/ | No | — | — | SINE | Alu | chr1: 109605629-109605927 | − | 14 | 299 | TAF13 | Proximal | 0 | Not documented | 9 |
| | Uniquely | | | | | | | | | | | | | | |
| AluSz6_dup2122 | Multi | No | — | — | SINE | Alu | chr1: 109606075-109606363 | − | 45 | 289 | TAF13 | Proximal | 0 | Not documented | 970 |
| MIRb_dup14129 | Multi | No | — | — | SINE | MIR | chr1: 109824039-109824175 | − | 120 | 137 | PSRC1 | Proximal | 0 | Not documented | 1 |
| MIRc_dup5840 | Multi | No | — | — | SINE | MIR | chr1: 109854537-109854636 | + | 150 | 100 | SORT1 | Proximal | 0 | Not documented | 2 |
| MLT2B2_dup95 | Uniquely | No | — | — | LTR | ERVL | chr1: 110207649-110208171 | + | 84 | 523 | GSTM4 | Proximal | 0 | Not documented | 3 |
| L1MC4a_dup908 | Multi/ | No | — | — | LINE | Other L1 | chr1: 110279109-110279365 | − | 170 | 257 | GSTM3 | Proximal | 0 | Not documented | 4 |
| | Uniquely | | | | | | | | | | | | | | |
| FLAM_A_dup742 | Multi/ | No | — | — | SINE | Alu | chr1: 110547260-110547384 | + | 95 | 125 | AHCYL1 | Proximal | 0 | Not documented | 5 |
| | Uniquely | | | | | | | | | | | | | | |
| MER 94B_dup31 | Multi/ | No | — | — | DNA | DNA | chr1: 110547638-110547737 | − | 94 | 100 | AHCYL1 | Proximal | 0 | Not documented | 6 |
| | Uniquely | | | | | | | | | | | | | | |
| AluSz6_dup2152 | Uniquely | No | — | — | SINE | Alu | chr1: 110563598-110563888 | + | 50 | 291 | AHCYL1 | Proximal | 0 | Not documented | 7 |
| MER5B_dup722 | Uniquely | No | — | — | DNA | DNA | chr1: 110563952-110564085 | − | 110 | 134 | AHCYL1 | Proximal | 0 | Not documented | 8 |
| L2c_dup8383 | Uniquely | No | — | — | LINE | L2 | chr1: 110604793-110605020 | + | 180 | 228 | ALX3 | Proximal | 0 | Not documented | 9 |
| Helitron2Na_Mam_{—} | Multi | No | — | — | Other | Other | chr1: 112003809-112004031 | + | 140 | 223 | ATP5F1 | Proximal | 0 | Not documented | 980 |
| dup17 | | | | | | Repeats | | | | | | | | | |
| MIRb_dup14385 | Uniquely | No | — | — | SINE | MIR | chr1: 112265506-112265704 | − | 130 | 199 | FAM212B | Proximal | 0 | Not documented | 1 |
| AluSx1_dup4762 | Multi/ | No | — | — | SINE | Alu | chr1: 112316032-112316342 | − | 54 | 311 | KCND3 | Proximal | 0 | Not documented | 2 |
| | Uniquely | | | | | | | | | | | | | | |
| MLT1A0_dup777 | Uniquely | No | — | — | LTR | ERVL- | chr1: 113153031-113153377 | − | 75 | 347 | ST7L | Proximal | 0 | Not documented | 3 |
| | | | | | | MaLR | | | | | | | | | |
| L2b_dup6263 | Multi | No | — | — | LINE | L2 | chr1: 113229119-113229680 | + | 140 | 562 | MOV10 | Proximal | 0 | Not documented | 4 |
| L1M1_dup311 | Uniquely | No | — | — | LINE | Other L1 | chr1: 114256401-114257995 | + | 57 | 1595 | PHTF1 | Proximal | 0 | Not documented | 5 |
| AluJb_dup7772 | Uniquely | No | — | — | SINE | Alu | chr1: 114257996-114258300 | + | 70 | 305 | PHTF1 | Proximal | 0 | Not documented | 6 |
| MIR_dup9819 | Multi | No | — | — | SINE | MIR | chr1: 115110014-115110264 | + | 120 | 251 | BCAS2 | Proximal | 0 | Not documented | 7 |
| AluSx3_dup1434 | Multi | No | — | — | SINE | Alu | chr1: 115248251-115248409 | − | 46 | 159 | NRAS | Proximal | 0 | Not documented | 8 |
| L3_dup2391 | Multi | No | — | — | LINE | CR1 | chr1: 116932866-116932914 | + | 81 | 49 | ATP1A1 | Proximal | 0 | Not documented | 9 |
| L2a_dup9377 | Uniquely | No | — | — | LINE | L2 | chr1: 117651831-117652786 | − | 130 | 956 | TRIM45 | Proximal | 897 | Not documented | 990 |
| L2c_dup9026 | Uniquely | No | — | — | LINE | L2 | chr1: 118423911-118424233 | − | 140 | 323 | GDAP2 | Proximal | 0 | Not documented | 1 |
| MIR3_dup5346 | Multi | No | — | — | SINE | MIR | chr1: 118502533-118502587 | − | 150 | 55 | WDR3 | Proximal | 0 | Not documented | 2 |
| AluSg_dup2030 | Uniquely | No | — | — | SINE | Alu | chr1: 119305589-119305908 | + | 53 | 320 | TBX15 | Distal | 119762 | Not documented | 3 |
| L2c_dup9163 | Multi | No | — | — | LINE | L2 | chr1: 120519146-120519788 | + | 140 | 643 | NOTCH2 | Proximal | 0 | Not documented | 4 |
| L1MB8_dup523 | Multi/ | No | — | — | LINE | Other L1 | chr1: 120531879-120533113 | + | 78 | 1235 | NOTCH2 | Proximal | 0 | Not documented | 5 |
| | Uniquely | | | | | | | | | | | | | | |
| MIR3_dup5433 | Multi/ | No | — | — | SINE | MIR | chr1: 120533196-120533341 | − | 150 | 146 | NOTCH2 | Proximal | 0 | Not documented | 6 |
| | Uniquely | | | | | | | | | | | | | | |
| AluSx1_dup5013 | Multi | No | — | — | SINE | Alu | chr1: 120534168-120534479 | + | 51 | 312 | NOTCH2 | Proximal | 0 | Not documented | 7 |
| L2b_dup6565 | Multi | No | — | — | LINE | L2 | chr1: 120534812-120535152 | − | 140 | 341 | NOTCH2 | Proximal | 0 | Not documented | 8 |
| AluSp_dup394 | Multi | No | — | — | SINE | Alu | chr1: 13919039-13919344 | − | 27 | 306 | PDPN | Proximal | 0 | Not documented | 9 |
| L1MB7_dup114 | Multi | No | — | — | LINE | Other L1 | chr1: 13919345-13919554 | − | 80 | 210 | PDPN | Proximal | 0 | Not documented | 1000 |
| AluSc_dup236 | Multi | No | — | — | SINE | Alu | chr1: 13943969-13944165 | − | 46 | 197 | PDPN | Proximal | 0 | Not documented | 1 |
| CER | Multi | No | — | — | Other | Other | chr1: 142653294-142657245 | − | 47 | 3952 | PPIAL4G | Distal | 1109900 | Not documented | 2 |
| | | | | | | Repeats | | | | | | | | | |
| CER_dup4 | Multi | No | — | — | Other | Other | chr1: 143434273-143438931 | + | 47 | 4659 | PPIAL4G | Distal | 328214 | Not documented | 3 |
| | | | | | | Repeats | | | | | | | | | |
| MER66C_dup33 | Multi | No | — | — | LTR | ERV1 | chr1: 143717471-143718011 | − | 88 | 541 | PPIAL4G | Distal | 49134 | Not documented | 4 |
| MER66-int_dup49 | Multi | No | — | — | LTR | ERV1 | chr1: 143718012-143718360 | − | 63 | 349 | PPIAL4G | Distal | 48785 | Not documented | 5 |
| L1MC4_dup1132 | Multi | No | — | — | LINE | Other L1 | chr1: 144829702-144830140 | − | 150 | 439 | NBPF9 | Proximal | 0 | Not documented | 6 |
| MIR3_dup5503 | Multi | No | — | — | SINE | MIR | chr1: 144984359-144984465 | − | 110 | 107 | PDE4DIP | Proximal | 0 | Not documented | 7 |
| AluJb_dup8156 | Multi | No | — | — | SINE | Alu | chr1: 145004877-145004999 | + | 70 | 123 | PDE4DIP | Proximal | 0 | Not documented | 8 |
| AluSp_dup2655 | Multi | No | — | — | SINE | Alu | chr1: 145005000-145005267 | + | 49 | 268 | PDE4DIP | Proximal | 0 | Not documented | 9 |
| AluSg_dup2069 | Multi | No | — | — | SINE | Alu | chr1: 145137496-145137781 | + | 46 | 286 | PDE4DIP | Distal | 61311 | Not documented | 1010 |
| AluSz_dup4839 | Multi | No | — | — | SINE | Alu | chr1: 145150328-145150658 | − | 51 | 331 | NOTCH2NL | Distal | 58462 | Not documented | 1 |
| AluSx1_dup5096 | Multi | No | — | — | SINE | Alu | chr1: 145286830-145287141 | − | 52 | 312 | NOTCH2NL | Proximal | 0 | Not documented | 2 |
| L1MB7_dup987 | Multi | No | — | — | LINE | Other L1 | chr1: 145287999-145289032 | − | 79 | 1034 | NOTCH2NL | Proximal | 0 | Not documented | 3 |
| L1MB7_dup988 | Multi | No | — | — | LINE | Other L1 | chr1: 145289021-145289266 | + | 98 | 246 | NOTCH2NL | Proximal | 0 | Not documented | 4 |
| AluJr_dup3603 | Multi | No | — | — | SINE | Alu | chr1: 145289363-145289667 | + | 70 | 305 | NOTCH2NL | Proximal | 0 | Not documented | 5 |
| MIR_dup10359 | Multi | No | — | — | SINE | MIR | chr1: 145289835-145289953 | + | 110 | 119 | NBPF10 | Proximal | 0 | Not documented | 6 |
| MER5A_dup1350 | Multi/ | No | — | — | DNA | DNA | chr1: 145289991-145290073 | + | 110 | 83 | NBPF10 | Proximal | 0 | Not documented | 7 |
| | Uniquely | | | | | | | | | | | | | | |
| L1MC4_dup1139 | Multi | No | — | — | LINE | Other L1 | chr1: 145369415-145369590 | + | 170 | 176 | NBPF10 | Proximal | 0 | Not documented | 8 |
| L1MC4_dup1140 | Multi | No | — | — | LINE | Other L1 | chr1: 145369602-145370040 | − | 150 | 439 | NBPF10 | Proximal | 0 | Not documented | 9 |
| MLT2F_dup82 | Multi | No | — | — | LTR | ERVL | chr1: 145384245-145384419 | − | 140 | 175 | NBPF10 | Distal | 13943 | Not documented | 1020 |
| AluSg7_dup414 | Multi | No | — | — | SINE | Alu | chr1: 145458500-145458791 | − | 51 | 292 | POLR3GL | Proximal | | Not documented | 1 |
| AluJb_dup8187 | Multi | No | — | — | SINE | Alu | chr1: 145495896-145496109 | + | 72 | 214 | LIX1L | Proximal | 0 | Not documented | 2 |
| AluSz6_dup2338 | Multi | No | — | — | SINE | Alu | chr1: 145499905-145500000 | − | 33 | 96 | LIX1L | Proximal | 0 | Not documented | 3 |
| AluSx_dup7382 | Multi | No | — | — | SINE | Alu | chr1: 145500001-145500203 | − | 43 | 203 | LIX1L | Proximal | 0 | Not documented | 4 |
| AluY_dup5684 | Multi | No | — | — | SINE | Alu | chr1: 145500214-145500489 | − | 30 | 276 | LIX1L | Proximal | | Not documented | 5 |
| AluY_dup5685 | Multi | No | — | — | SINE | Alu | chr1: 145500501-145500802 | − | 16 | 302 | LIX1L | Proximal | 0 | Not documented | 6 |
| AluSz6_dup2339 | Multi | No | — | — | SINE | Alu | chr1: 145512872-145513145 | − | 60 | 274 | RBM8A | Proximal | 0 | Not documented | 7 |
| L1PA15_dup22 | Uniquely | No | — | — | LINE | L1PA\|B\|x | chr1: 14551485-14553544 | + | 55 | 2060 | KAZN | Distal | 371657 | Not documented | 8 |
| L1MC4_dup1145 | Multi | No | — | — | LINE | Other L1 | chr1: 146032809-146033247 | + | 140 | 439 | NBPF11 | Proximal | 0 | Not documented | 9 |
| L1MC4_dup1146 | Multi | No | — | — | LINE | Other L1 | chr1: 146033259-146033434 | − | 170 | 176 | NBPF11 | Proximal | | Not documented | 1030 |
| L1MC4_dup1147 | Multi | No | — | — | LINE | Other L1 | chr1: 146466852-146467027 | + | 170 | 176 | NBPF12 | Proximal | | Not documented | 1 |
| L1MC4_dup1148 | Multi | No | — | — | LINE | Other L1 | chr1: 146467039-146467477 | − | 150 | 439 | NBPF12 | Proximal | | Not documented | 2 |
| L2b_dup6674 | Multi/ | No | — | — | LINE | L2 | chr1: 147079473-147080358 | − | 130 | 886 | BCL9 | Proximal | | Not documented | 3 |
| | Uniquely | | | | | | | | | | | | | | |
| MIR_dup10454 | Multi/ | No | — | — | SINE | MIR | chr1: 147080640-147080861 | + | 110 | 222 | BCL9 | Proximal | | Not documented | 4 |
| | Uniquely | | | | | | | | | | | | | | |
| L1MC4_dup1163 | Multi | No | — | — | LINE | Other L1 | chr1: 147574590-147575028 | + | 140 | 439 | NBPF24 | Proximal | 0 | Not documented | 5 |
| L1MC4_dup1164 | Multi | No | — | — | LINE | Other L1 | chr1: 147575040-147575215 | − | 170 | 176 | NBPF24 | Proximal | 0 | Not documented | 6 |
| MER66C_dup35 | Multi | No | — | — | LTR | ERV1 | chr1: 147904980-147905520 | − | 88 | 541 | PPIAL4A | Distal | 49116 | Not documented | 7 |
| MER66-int_dup54 | Multi | No | — | — | LTR | ERV1 | chr1: 147905521-147905869 | − | 63 | 349 | PPIAL4A | Distal | 48767 | Not documented | 8 |
| L1MC4_dup1168 | Multi | No | — | — | LINE | Other L1 | chr1: 148003192-148003630 | + | 150 | 439 | NBPF14 | Proximal | 13 | Not documented | 9 |
| L1MC4_dup1169 | Multi | No | — | — | LINE | Other L1 | chr1: 148003642-148003817 | − | 160 | 176 | NBPF14 | Proximal | 0 | Not documented | 1040 |
| L1MC4_dup1171 | Multi | No | — | — | LINE | Other L1 | chr1: 148250516-148250957 | + | 150 | 442 | NBPF20 | Proximal | 0 | Not documented | 1 |
| L1MC4_dup1172 | Multi | No | — | — | LINE | Other L1 | chr1: 148251044-148251144 | − | 150 | 101 | NBPF20 | Proximal | 0 | Not documented | 2 |
| AluJb_dup8341 | Multi | No | — | — | SINE | Alu | chr1: 148558146-148558443 | − | 72 | 298 | NBPF15 | Proximal | 0 | Not documented | 3 |
| L1MC4_dup1173 | Multi | No | — | — | LINE | Other L1 | chr1: 148595371-148595546 | + | 170 | 176 | NBPF15 | Proximal | 0 | Not documented | 4 |
| L1MC4_dup1174 | Multi | No | — | — | LINE | Other L1 | chr1: 148595558-148595996 | − | 150 | 439 | NBPF15 | Proximal | 0 | Not documented | 5 |
| L1MC4_dup1177 | Multi | No | — | — | LINE | Other L1 | chr1: 148757415-148757590 | + | 170 | 176 | NBPF16 | Proximal | 0 | Not documented | 6 |
| L1MC4_dup1178 | Multi | No | — | — | LINE | Other L1 | chr1: 148757602-148758040 | − | 150 | 439 | NBPF16 | Proximal | 0 | Not documented | 7 |
| TAR1_dup5 | Multi | No | — | — | Other | Other | chr1: 148927800-148928362 | − | 74 | 563 | NBPF16 | Distal | 169490 | Not documented | 8 |
| | | | | | | Repeats | | | | | | | | | |
| L1MEc_dup988 | Multi | No | — | — | LINE | Other L1 | chr1: 148936741-148938004 | + | 89 | 1264 | NBPF16 | Distal | 178431 | Not documented | 9 |
| MLT1J2_dup325 | Multi | No | — | — | LTR | ERVL- | chr1: 148951232-148951367 | + | 140 | 136 | NBPF16 | Distal | 192922 | Not documented | 1050 |
| | | | | | | MaLR | | | | | | | | | |
| LTR4_dup6 | Multi | No | — | — | LTR | ERV1 | chr1: 149214757-149215159 | − | 95 | 403 | HIST2H3PS2 | Distal | 184973 | Not documented | 1 |
| Charlie23a_dup82 | Multi | No | — | — | DNA | DNA | chr1: 149855935-149856163 | − | 110 | 229 | HIST2H2BE | Proximal | 0 | Not documented | 2 |
| MIRc_dup6637 | Multi | No | — | — | SINE | MIR | chr1: 149856309-149856433 | + | 160 | 125 | HIST2H2BE | Proximal | 0 | Not documented | 3 |
| MER3_dup488 | Uniquely | No | — | — | DNA | DNA | chr1: 150312988-150313077 | + | 140 | 90 | PRPF3 | Proximal | 0 | Not documented | 4 |
| AluSc8_dup1079 | Multi | No | — | — | SINE | Alu | chr1: 150479764-150480068 | + | 34 | 305 | TARS2 | Proximal | 0 | Not documented | 5 |
| MIRb_dup15563 | Multi | No | — | — | SINE | MIR | chr1: 150597273-150597450 | − | 130 | 178 | ENSA | Proximal | 0 | Not documented | 6 |
| AluSz_dup5111 | Uniquely | No | — | — | SINE | Alu | chr1: 151171807-151172107 | + | 53 | 301 | PIP5K1A | Proximal | 0 | Not documented | 7 |
| L3_dup2615 | Multi | No | — | — | LINE | CR1 | chr1: 151373177-151373244 | − | 120 | 68 | PSMB4 | Proximal | 0 | Not documented | 8 |
| AluJr4_dup1025 | Multi | No | — | — | SINE | Alu | chr1: 151667887-151668196 | − | 59 | 310 | SNX27 | Proximal | 0 | Not documented | 9 |
| AluSz_dup5157 | Multi | No | — | — | SINE | Alu | chr1: 151669118-151669425 | − | 47 | 308 | SNX27 | Proximal | 0 | Not documented | 1060 |
| AluSg_dup2235 | Multi | No | — | — | SINE | Alu | chr1: 153609849-153610141 | + | 39 | 293 | CHTOP | Proximal | 0 | Not documented | 1 |
| MIRb_dup15879 | Multi/ | No | — | — | SINE | MIR | chr1: 154916551-154916718 | − | 150 | 168 | PBXIP1 | Proximal | 0 | Not documented | 2 |
| | Uniquely | | | | | | | | | | | | | | |
| HAL1_dup7 | Multi | No | — | — | LINE | Other L1 | chr1: 1549173-1549465 | + | 160 | 293 | MIB2 | Proximal | 1331 | Not documented | 3 |
| MIRc_dup6826 | Multi/ | No | — | — | SINE | MIR | chr1: 154935130-154935225 | + | 130 | 96 | PYGO2 | Proximal | 0 | Not documented | 4 |
| | Uniquely | | | | | | | | | | | | | | |
| MER65D_dup11 | Multi | No | — | — | LTR | ERV1 | chr1: 1549546-1550009 | + | 150 | 464 | MIB2 | Proximal | 787 | Not documented | 5 |
| AluSg_dup40 | Multi | No | — | — | SINE | Alu | chr1: 1550115-1550424 | + | 51 | 310 | MIB2 | Proximal | 372 | Not documented | 6 |
| L2c_dup9821 | Multi | No | — | — | LINE | L2 | chr1: 155175200-155175275 | + | 120 | 76 | THBS3 | Proximal | 0 | Not documented | 7 |
| MIRb_dup15921 | Multi | No | — | — | SINE | MIR | chr1: 155584675-155584858 | − | 130 | 184 | MSTO1 | Proximal | 0 | Not documented | 8 |
| HERVK-int_dup15 | Multi | No | — | — | LTR | ERVK | chr1: 155599135-155604668 | − | 3.6 | 5534 | MSTO1 | Proximal | 0 | EVE annotated | 9 |
| AluSq2_dup3062 | Multi | No | — | — | SINE | Alu | chr1: 155851514-155851819 | − | 48 | 306 | SYT11 | Proximal | 0 | Not documented | 1070 |
| MIR3_dup5829 | Multi | No | — | — | SINE | MIR | chr1: 155852942-155852973 | − | 100 | 32 | SYT11 | Proximal | 0 | Not documented | 1 |
| AluJb_dup9036 | Multi | No | — | — | SINE | Alu | chr1: 155853043-155853327 | + | 65 | 285 | SYT11 | Proximal | 0 | Not documented | 2 |
| AluSz_dup5421 | Multi/ | No | — | — | SINE | Alu | chr1: 156374430-156374721 | − | 48 | 292 | C1orf61 | Proximal | 0 | Not documented | 3 |
| | Uniquely | | | | | | | | | | | | | | |
| L3b_dup331 | Multi/ | No | — | — | LINE | CR1 | chr1: 156376864-156377001 | + | 150 | 138 | C1orf61 | Proximal | 0 | Not documented | 4 |
| | Uniquely | | | | | | | | | | | | | | |
| MIR3_dup5861 | Multi/ | No | — | — | SINE | MIR | chr1: 156383997-156384142 | + | 120 | 146 | C1orf61 | Proximal | 0 | Not documented | 5 |
| | Uniquely | | | | | | | | | | | | | | |
| L3_dup2777 | Multi/ | No | — | — | LINE | CR1 | chr1: 160184145-160184202 | − | 110 | 58 | PEA15 | Proximal | 0 | Not documented | 6 |
| | Uniquely | | | | | | | | | | | | | | |
| MER113A_dup8 | Multi/ | No | — | — | DNA | DNA | chr1: 16112873-16113080 | − | 95 | 208 | FBLIM1 | Proximal | 0 | Not documented | 7 |
| | Uniquely | | | | | | | | | | | | | | |
| AluSc8_dup1213 | Uniquely | No | — | — | SINE | Alu | chr1: 161396886-161397190 | − | 30 | 305 | C1orf192 | Distal | 59223 | Not documented | 8 |
| AluYa5_dup206 | Multi/ | No | — | — | SINE | Alu | chr1: 162259255-162259564 | − | 31 | 310 | NOS1AP | Proximal | 0 | Not documented | 9 |
| | Uniquely | | | | | | | | | | | | | | |
| SVA_A_dup11 | Multi | No | — | — | Other | SVA | chr1: 162427364-162427708 | + | 8.2 | 345 | UHMK1 | Distal | 39334 | Not documented | 1080 |
| AluSz_dup5640 | Multi/ | No | — | — | SINE | Alu | chr1: 162536421-162536717 | − | 52 | 297 | UAP1 | Proximal | 0 | Not documented | 1 |
| | Uniquely | | | | | | | | | | | | | | |
| L2c_dup10303 | Multi | No | — | — | LINE | L2 | chr1: 162537072-162537193 | + | 150 | 122 | UAP1 | Proximal | 0 | Not documented | 2 |
| AluJb_dup9400 | Multi | No | — | — | SINE | Alu | chr1: 162547227-162547405 | − | 77 | 179 | UAP1 | Proximal | 0 | Not documented | 3 |
| AluSg_dup2453 | Multi | No | — | — | SINE | Alu | chr1: 162547406-162547698 | − | 48 | 293 | UAP1 | Proximal | 0 | Not documented | 4 |
| AluJb_dup9401 | Multi | No | — | — | SINE | Alu | chr1: 162547699-162547820 | − | 77 | 122 | UAP1 | Proximal | 0 | Not documented | 5 |
| L1MEg1_dup37 | Multi | No | — | — | LINE | Other L1 | chr1: 162547822-162548265 | − | 120 | 444 | UAP1 | Proximal | 0 | Not documented | 6 |
| AluJb_dup9411 | Multi | No | — | — | SINE | Alu | chr1: 162782183-162782469 | + | 65 | 287 | HSD17B7 | Proximal | 0 | Not documented | 7 |
| L2c_dup10459 | Uniquely | No | — | — | LINE | L2 | chr1: 164664557-164664964 | + | 160 | 408 | PBX1 | Proximal | 0 | Not documented | 8 |
| L1MD1_dup33 | Multi/ | No | — | — | LINE | Other L1 | chr1: 16798444-16800584 | − | 96 | 2141 | NECAP2 | Distal | 11872 | Not documented | 9 |
| | Uniquely | | | | | | | | | | | | | | |
| L2c_dup10853 | Uniquely | No | — | — | LINE | L2 | chr1: 169352393-169352975 | + | 150 | 583 | BLZF1 | Proximal | 0 | Not documented | 1090 |
| L2a_dup1104 | Multi/ | No | — | — | LINE | L2 | chr1: 16935252-16935392 | − | 130 | 141 | NBPF1 | Proximal | 0 | Not documented | 1 |
| | Uniquely | | | | | | | | | | | | | | |
| L1M5_dup329 | Multi | No | — | — | LINE | Other L1 | chr1: 16944983-16945142 | + | 160 | 160 | NBPF1 | Distal | 4927 | Not documented | 2 |
| AluSx_dup1703 | Multi | No | — | — | SINE | Alu | chr1: 16961526-16961832 | − | 53 | 307 | NBPF1 | Distal | 21470 | Not documented | 3 |
| AluSg_dup2536 | Multi/ | No | — | — | SINE | Alu | chr1: 170635282-170635581 | + | 36 | 300 | PRRX1 | Proximal | 0 | Not documented | 4 |
| | Uniquely | | | | | | | | | | | | | | |
| L2_dup3436 | Uniquely | No | — | — | LINE | L2 | chr1: 170642614-170643085 | + | 150 | 472 | PRRX1 | Proximal | 0 | Not documented | 5 |
| MIRb_dup17226 | Multi/ | No | — | — | SINE | MIR | chr1: 170650250-170650437 | − | 160 | 188 | PRRX1 | Proximal | 0 | Not documented | 6 |
| | Uniquely | | | | | | | | | | | | | | |
| LFSINE_Vert_dup52 | Multi | No | — | — | SINE | SINE | chr1: 170655174-170655263 | + | 88 | 90 | PRRX1 | Proximal | 0 | Not documented | 7 |
| AluJo_dup4402 | Multi | No | — | — | SINE | Alu | chr1: 173833126-173833236 | + | 78 | 111 | ZBTB37 | Distal | 3985 | Not documented | 8 |
| AluSx_dup9051 | Multi | No | — | — | SINE | Alu | chr1: 173905495-173905680 | + | 44 | 186 | RC3H1 | Proximal | 0 | Not documented | 9 |
| L2_dup3560 | Multi/ | No | — | — | LINE | L2 | chr1: 174982259-174982811 | + | 160 | 553 | MRPS14 | Proximal | 0 | Not documented | 1100 |
| | Uniquely | | | | | | | | | | | | | | |
| L2b_dup7644 | Multi | No | — | — | LINE | L2 | chr1: 175127427-175127490 | − | 110 | 64 | KIAA0040 | Proximal | 0 | Not documented | 1 |
| Charlie21a_dup54 | Multi | No | — | — | DNA | DNA | chr1: 179810301-179810618 | − | 120 | 318 | TOR1AIP2 | Proximal | 0 | Not documented | 2 |
| AluJb_dup10132 | Multi | No | — | — | SINE | Alu | chr1: 179810619-179810765 | + | 74 | 147 | TOR1AIP2 | Proximal | 0 | Not documented | 3 |
| Charlie21a_dup55 | Multi | No | — | — | DNA | DNA | chr1: 179810766-179810947 | − | 110 | 182 | TOR1AIP2 | Proximal | 0 | Not documented | 4 |
| AluSp_dup3422 | Multi/ | No | — | — | SINE | Alu | chr1: 179811054-179811350 | + | 41 | 297 | TOR1AIP2 | Proximal | 0 | Not documented | 5 |
| | Uniquely | | | | | | | | | | | | | | |
| Charlie21a_dup57 | Multi | No | — | — | DNA | DNA | chr1: 179811351-179811458 | − | 130 | 108 | TOR1AIP2 | Proximal | 0 | Not documented | 6 |
| AluY_dup7152 | Multi/ | No | — | — | SINE | Alu | chr1: 179811898-179812205 | + | 32 | 308 | TOR1AIP2 | Proximal | 0 | Not documented | 7 |
| | Uniquely | | | | | | | | | | | | | | |
| AluSz6_dup2938 | Multi | No | — | — | SINE | Alu | chr1: 179812359-179812670 | + | 61 | 312 | TOR1AIP2 | Proximal | 0 | Not documented | 8 |
| AluSq2_dup3456 | Multi | No | — | — | SINE | Alu | chr1: 179888160-179888412 | + | 43 | 253 | TOR1AIP1 | Proximal | 0 | Not documented | 9 |
| AluY_dup7199 | Multi | No | — | — | SINE | Alu | chr1: 180866853-180867161 | − | 34 | 309 | XPR1 | Distal | 7467 | Not documented | 1110 |
| L1MC3_dup556 | Uniquely | No | — | — | LINE | Other L1 | chr1: 182842573-182843481 | + | 96 | 909 | DHX9 | Proximal | 0 | Not documented | 1 |
| MER58A_dup641 | Multi/ | No | — | — | DNA | DNA | chr1: 184042850-184043018 | − | 71 | 169 | TSEN 15 | Proximal | 0 | Not documented | 2 |
| | Uniquely | | | | | | | | | | | | | | |
| MLT1B_dup850 | Multi/ | No | — | — | LTR | ERVL- | chr1: 184064559-184065022 | + | 95 | 464 | TSEN15 | Distal | 21214 | Not documented | 3 |
| | Uniquely | | | | | MaLR | | | | | | | | | |
| L1MA3_dup438 | Uniquely | No | — | — | LINE | Other L1 | chr1: 184892038-184894185 | − | 51 | 2148 | FAM129A | Proximal | 0 | Not documented | 4 |
| AluJr_dup4676 | Multi/ | No | — | — | SINE | Alu | chr1: 186388563-186388876 | + | 93 | 314 | C1orf27 | Proximal | 0 | Not documented | 5 |
| | Uniquely | | | | | | | | | | | | | | |
| L1ME3F_dup322 | Multi | No | — | — | LINE | Other L1 | chr1: 186389698-186390715 | + | 130 | 1018 | C1orf27 | Proximal | 0 | Not documented | 6 |
| AluYa5_dup242 | Multi | No | — | — | SINE | Alu | chr1: 187132806-187133116 | + | 2.7 | 311 | PLA2G4A | Distal | 174694 | Not documented | 7 |
| AluJb_dup10597 | Multi | No | — | — | SINE | Alu | chr1: 193057151-193057441 | − | 78 | 291 | TROVE2 | Proximal | 0 | Not documented | 8 |
| AluSc5_dup410 | Multi | No | — | — | SINE | Alu | chr1: 193058933-193059233 | − | 36 | 301 | TROVE2 | Proximal | 0 | Not documented | 9 |
| Kangalc_dup50 | Multi | No | — | — | DNA | DNA | chr1: 193199577-193199889 | + | 100 | 313 | CDC73 | Proximal | 0 | Not documented | 1120 |
| AluSg_dup2795 | Uniquely | No | — | — | SINE | Alu | chr1: 194376637-194376963 | + | 51 | 327 | CDC73 | Distal | 1153607 | Not documented | 1 |
| AluSc8_dup239 | Multi | No | — | — | SINE | Alu | chr1: 19585475-19585774 | + | 51 | 300 | MRTO4 | Proximal | 0 | Not documented | 2 |
| Tigger1_dup750 | Multi/ | No | — | — | DNA | DNA | chr1: 200755810-200757241 | + | 59 | 1432 | CAMSAP2 | Proximal | 0 | Not documented | 3 |
| | Uniquely | | | | | | | | | | | | | | |
| AluSz_dup6657 | Uniquely | No | — | — | SINE | Alu | chr1: 201831304-201831594 | + | 49 | 291 | IPO9 | Proximal | 0 | Not documented | 4 |
| MIRb_dup19369 | Multi | No | — | — | SINE | MIR | chr1: 202100439-202100555 | + | 110 | 117 | GPR37L1 | Proximal | 0 | Not documented | 5 |
| L1ME3B_dup476 | Uniquely | No | — | — | LINE | Other L1 | chr1: 203145828-203146537 | + | 130 | 710 | MYBPH | Proximal | 888 | Not documented | 6 |
| L3_dup3627 | Multi/ | No | — | — | LINE | CR1 | chr1: 203147496-203147658 | + | 160 | 163 | CHI3L1 | Proximal | 402 | Not documented | 7 |
| | Uniquely | | | | | | | | | | | | | | |
| MIRb_dup19475 | Multi/ | No | — | — | SINE | MIR | chr1: 203149262-203149337 | − | 110 | 76 | CHI3L1 | Proximal | 0 | Not documented | 8 |
| | Uniquely | | | | | | | | | | | | | | |
| MIRb_dup19476 | Multi/ | No | — | — | SINE | MIR | chr1: 203150139-203150228 | + | 120 | 90 | CHI3L1 | Proximal | 0 | Not documented | 9 |
| | Uniquely | | | | | | | | | | | | | | |
| MIRb_dup19477 | Multi/ | No | — | — | SINE | MIR | chr1: 203153071-203153277 | + | 120 | 207 | CHI3L1 | Proximal | 0 | Not documented | 1130 |
| | Uniquely | | | | | | | | | | | | | | |
| MIR3_dup7484 | Multi | No | — | — | SINE | MIR | chr1: 203766281-203766401 | + | 110 | 121 | ZBED6 | Proximal | 0 | Not documented | 1 |
| AluJo_dup5072 | Uniquely | No | — | — | SINE | Alu | chr1: 204502896-204503211 | − | 76 | 316 | MDM4 | Proximal | 0 | Not documented | 2 |
| AluJb_dup11187 | Uniquely | No | — | — | SINE | Alu | chr1: 204503618-204503919 | − | 78 | 302 | MDM4 | Proximal | 0 | Not documented | 3 |
| AluY_dup8009 | Multi | No | — | — | SINE | Alu | chr1: 204504283-204504584 | − | 36 | 302 | MDM4 | Proximal | 0 | Not documented | 4 |
| MER5B_dup1247 | Multi | No | — | — | DNA | DNA | chr1: 205737400-205737532 | − | 150 | 133 | RAB7L1 | Proximal | 0 | Not documented | 5 |
| AluSq2_dup3870 | Multi | No | — | — | SINE | Alu | chr1: 205737654-205737961 | − | 49 | 308 | RAB7L1 | Proximal | 0 | Not documented | 6 |
| MER5B_dup1248 | Multi | No | — | — | DNA | DNA | chr1: 205737997-205738047 | − | 110 | 51 | RAB7L1 | Proximal | 0 | Not documented | 7 |
| AluSz6_dup3299 | Multi | No | — | — | SINE | Alu | chr1: 205738555-205738856 | − | 57 | 302 | RAB7L1 | Proximal | 0 | Not documented | 8 |
| L3_dup3706 | Multi/ | No | — | — | LINE | CR1 | chr1: 205758780-205758828 | + | 100 | 49 | SLC41A1 | Proximal | 0 | Not documented | 9 |
| | Uniquely | | | | | | | | | | | | | | |
| L2a_dup13651 | Multi/ | No | — | — | LINE | L2 | chr1: 205759389-205759498 | + | 120 | 110 | SLC41A1 | Proximal | 0 | Not documented | 1140 |
| | Uniquely | | | | | | | | | | | | | | |
| L3b_dup591 | Uniquely | No | — | — | LINE | CR1 | chr1: 208196071-208196214 | + | 170 | 144 | PLXNA2 | Proximal | 0 | Not documented | 1 |
| AluSc8_dup253 | Multi | No | — | — | SINE | Alu | chr1: 21069875-21070183 | + | 57 | 309 | HP1BP3 | Proximal | 0 | Not documented | 2 |
| AluSx_dup2021 | Multi/ | No | — | — | SINE | Alu | chr1: 21070279-21070559 | + | 41 | 281 | HP1BP3 | Proximal | 0 | Not documented | 3 |
| | Uniquely | | | | | | | | | | | | | | |
| Tigger3b_dup19 | Uniquely | No | — | — | DNA | DNA | chr1: 21101499-21102429 | + | 75 | 931 | HP1BP3 | Proximal | 0 | Not documented | 4 |
| L1ME4a_dup2854 | Multi | No | — | — | LINE | Other L1 | chr1: 212619600-212619696 | + | 100 | 97 | NENF | Proximal | 0 | Not documented | 5 |
| MIRc_dup9314 | Multi | No | — | — | SINE | MIR | chr1: 212988640-212988716 | + | 130 | 77 | TATDN3 | Proximal | 0 | Not documented | 6 |
| AluSp_dup3913 | Multi | No | — | — | SINE | Alu | chr1: 212988871-212989159 | − | 31 | 289 | TATDN3 | Proximal | 0 | Not documented | 7 |
| FRAM_dup89 | Uniquely | No | — | — | SINE | Alu | chr1: 21439506-21439659 | + | 74 | 154 | EIF4G3 | Proximal | 0 | Not documented | 8 |
| AluJb_dup11681 | Uniquely | No | — | — | SINE | Alu | chr1: 214529295-214529600 | − | 67 | 306 | PTPN14 | Proximal | 0 | Not documented | 9 |
| AluSx1_dup1312 | Multi/ | No | — | — | SINE | Alu | chr1: 21545079-21545383 | − | 40 | 305 | ECE1 | Proximal | 0 | Not documented | 1150 |
| | Uniquely | | | | | | | | | | | | | | |
| L2c_dup13877 | Uniquely | No | — | — | LINE | L2 | chr1: 217037057-217037166 | + | 140 | 110 | ESRRG | Proximal | 0 | Not documented | 1 |
| L1MC4_dup213 | Multi | No | — | — | LINE | Other L1 | chr1: 21749756-21750197 | + | 150 | 442 | NBPF3 | Distal | 16425 | Not documented | 2 |
| L1MC4_dup214 | Multi | No | — | — | LINE | Other L1 | chr1: 21750268-21750382 | − | 140 | 115 | NBPF3 | Distal | 16240 | Not documented | 3 |
| L1MC4_dup217 | Multi | No | — | — | LINE | Other L1 | chr1: 21810796-21811237 | − | 150 | 442 | NBPF3 | Proximal | 0 | Not documented | 4 |
| AluSx4_dup447 | Uniquely | No | — | — | SINE | Alu | chr1: 218534198-218534494 | − | 49 | 297 | TGFB2 | Proximal | 0 | Not documented | 5 |
| AluSx1_dup7648 | Uniquely | No | — | — | SINE | Alu | chr1: 218588755-218589066 | + | 38 | 312 | TGFB2 | Proximal | 0 | Not documented | 6 |
| AluY_dup8511 | Multi | No | — | — | SINE | Alu | chr1: 219372952-219373178 | − | 28 | 227 | LYPLAL1 | Proximal | 0 | Not documented | 7 |
| L1MB1_dup347 | Uniquely | No | — | — | LINE | Other L1 | chr1: 220802680-220803743 | − | 70 | 1064 | MARK1 | Proximal | 0 | Not documented | 8 |
| L1PA10_dup383 | Uniquely | No | — | — | LINE | L1PA\|B\|x | chr1: 220814955-220821404 | + | 45 | 6450 | MARK1 | Proximal | 0 | Not documented | 9 |
| L5_dup71 | Multi | No | — | — | LINE | RTE | chr1: 222907339-222907421 | − | 82 | 83 | BROX | Proximal | 0 | Not documented | 1160 |
| MIR_dup15020 | Multi | No | — | — | SINE | MIR | chr1: 222907573-222907676 | − | 120 | 104 | BROX | Proximal | 0 | Not documented | 1 |
| AluSx_dup10986 | Multi/ | No | — | — | SINE | Alu | chr1: 223945555-223945860 | − | 50 | 306 | CAPN2 | Proximal | 0 | Not documented | 2 |
| | Uniquely | | | | | | | | | | | | | | |
| AluJr_dup5419 | Multi/ | No | — | — | SINE | Alu | chr1: 223946392-223946687 | − | 66 | 296 | CAPN2 | Proximal | 0 | Not documented | 3 |
| | Uniquely | | | | | | | | | | | | | | |
| AluJr_dup5420 | Multi | No | — | — | SINE | Alu | chr1: 223946785-223946839 | − | 99 | 55 | CAPN2 | Proximal | 0 | Not documented | 4 |
| AluSp_dup4068 | Uniquely | No | — | — | SINE | Alu | chr1: 224356032-224356326 | − | 43 | 295 | FBXO28 | Distal | 6284 | Not documented | 5 |
| AluSx_dup11043 | Multi | No | — | — | SINE | Alu | chr1: 224380475-224380793 | + | 50 | 319 | DEGS1 | Proximal | 0 | Not documented | 6 |
| MIRc_dup9799 | Multi | No | — | — | SINE | MIR | chr1: 225836788-225836922 | − | 100 | 135 | ENAH | Proximal | 0 | Not documented | 7 |
| AluSc8_dup1593 | Multi | No | — | — | SINE | Alu | chr1: 225838271-225838580 | + | 35 | 310 | ENAH | Proximal | 0 | Not documented | 8 |
| AluJo_dup5542 | Multi/ | No | — | — | SINE | Alu | chr1: 225974548-225974834 | − | 78 | 287 | SRP9 | Proximal | 0 | Not documented | 9 |
| | Uniquely | | | | | | | | | | | | | | |
| AluSx1_dup7873 | Multi | No | — | — | SINE | Alu | chr1: 225974955-225975248 | + | 43 | 294 | SRP9 | Proximal | 0 | Not documented | 1170 |
| MamRep434_dup171 | Multi | No | — | — | DNA | DNA | chr1: 226820238-226820561 | − | 130 | 324 | ITPKB | Proximal | 0 | Not documented | 1 |
| L2_dup4754 | Uniquely | No | — | — | LINE | L2 | chr1: 227191874-227192341 | + | 150 | 468 | CDC42BPA | Proximal | 0 | Not documented | 2 |
| L1MB4_dup524 | Uniquely | No | — | — | LINE | Other L1 | chr1: 227293355-227295109 | − | 99 | 1755 | CDC42BPA | Proximal | 0 | Not documented | 3 |
| L1MA2_dup448 | Uniquely | No | — | — | LINE | Other L1 | chr1: 227435928-227437530 | + | 64 | 1603 | CDC42BPA | Proximal | 0 | Not documented | 4 |
| X3_LINE_dup59 | Uniquely | No | — | — | LINE | RTE-BovB | chr1: 227494088-227494223 | + | 160 | 136 | CDC42BPA | Proximal | 0 | Not documented | 5 |
| MER31-int_dup2 | Multi | No | — | — | LTR | ERV1 | chr1: 227779-228217 | + | 99 | 439 | AL627309.1 | Distal | 88401 | Not documented | 6 |
| MER5A_dup2286 | Multi/ | No | — | — | DNA | DNA | chr1: 228296166-228296251 | − | 130 | 86 | MRPL55 | Proximal | 0 | Not documented | 7 |
| | Uniquely | | | | | | | | | | | | | | |
| MIR3_dup8441 | Multi | No | — | — | SINE | MIR | chr1: 229577273-229577383 | − | 120 | 111 | NUP133 | Proximal | 0 | Not documented | 8 |
| L1MC1_dup1013 | Multi | No | — | — | LINE | Other L1 | chr1: 230315964-230317304 | − | 79 | 1341 | GALNT2 | Proximal | 0 | Not documented | 9 |
| AluSz6_dup3723 | Multi | No | — | — | SINE | Alu | chr1: 231501186-231501406 | + | 64 | 221 | EGLN1 | Proximal | 0 | Not documented | 1180 |
| AluSg_dup3319 | Multi | No | — | — | SINE | Alu | chr1: 231701190-231701495 | + | 50 | 306 | TSNAX | Proximal | 0 | Not documented | 1 |
| L2a_dup87 | Multi | No | — | — | LINE | L2 | chr1: 2324663-2324938 | − | 160 | 276 | RER1 | Proximal | 0 | Not documented | 2 |
| MER 131_dup42 | Multi | No | — | — | SINE | SINE | chr1: 234741371-234741443 | − | 100 | 73 | IRF2BP2 | Proximal | 0 | Not documented | 3 |
| MIR3_dup8588 | Multi | No | — | — | SINE | MIR | chr1: 235273856-235273983 | − | 160 | 128 | TOMM20 | Proximal | 0 | Not documented | 4 |
| L1ME3C_dup850 | Multi | No | — | — | LINE | Other L1 | chr1: 235451661-235452048 | − | 150 | 388 | ARID4B | Proximal | 0 | Not documented | 5 |
| L1MC_dup734 | Multi | No | — | — | LINE | Other L1 | chr1: 235452320-235452444 | + | 120 | 125 | ARID4B | Proximal | 0 | Not documented | 6 |
| L1MC4a_dup1782 | Multi | No | — | — | LINE | Other L1 | chr1: 235452707-235452758 | + | 100 | 52 | ARID4B | Proximal | 0 | Not documented | 7 |
| AluJb_dup12701 | Multi | No | — | — | SINE | Alu | chr1: 235452759-235453056 | + | 81 | 298 | ARID4B | Proximal | 0 | Not documented | 8 |
| L1PB4_dup413 | Uniquely | No | — | — | LINE | L1PA\|B\|x | chr1: 235495391-235495852 | − | 82 | 462 | GGPS1 | Proximal | 0 | Not documented | 9 |
| AluY_dup9186 | Uniquely | No | — | — | SINE | Alu | chr1: 235506589-235506873 | + | 18 | 285 | GGPS1 | Proximal | 0 | Not documented | 1190 |
| MIR3_dup8592 | Multi | No | — | — | SINE | MIR | chr1: 235610844-235610932 | − | 87 | 89 | TBCE | Proximal | 0 | Not documented | 1 |
| AluSg_dup3385 | Multi | No | — | — | SINE | Alu | chr1: 235611246-235611555 | + | 37 | 310 | TBCE | Proximal | 0 | Not documented | 2 |
| MIR_dup15667 | Multi | No | — | — | SINE | MIR | chr1: 235714126-235714208 | + | 110 | 83 | GNG4 | Proximal | 0 | Not documented | 3 |
| AluSx3_dup2369 | Multi/ | No | — | — | SINE | Alu | chr1: 235714562-235714872 | + | 42 | 311 | GNG4 | Proximal | 0 | Not documented | 4 |
| | Uniquely | | | | | | | | | | | | | | |
| MER112_dup21 | Multi | No | — | — | DNA | DNA | chr1: 23647185-23647332 | + | 110 | 148 | HNRNPR | Proximal | 0 | Not documented | 5 |
| AluSp_dup685 | Multi/ | No | — | — | SINE | Alu | chr1: 23647379-23647670 | + | 31 | 292 | HNRNPR | Proximal | 0 | Not documented | 6 |
| | Uniquely | | | | | | | | | | | | | | |
| MamRep4096_dup6 | Multi/ | No | — | — | DNA | DNA | chr1: 23698046-23698432 | − | 110 | 387 | C1orf213 | Proximal | | Not documented | 7 |
| | Uniquely | | | | | | | | | | | | | | |
| MamRep1894_dup2 | Multi/ | No | — | — | DNA | DNA | chr1: 23755632-23755749 | + | 150 | 118 | ASAP3 | Proximal | 0 | Not documented | 8 |
| | Uniquely | | | | | | | | | | | | | | |
| L2b_dup1328 | Multi | No | — | — | LINE | L2 | chr1: 23755832-23755927 | + | 170 | 96 | ASAP3 | Proximal | 0 | Not documented | 9 |
| MIRc_dup1241 | Multi/ | No | — | — | SINE | MIR | chr1: 23885270-23885338 | + | 87 | 69 | IDE | Proximal | 0 | Not documented | 1200 |
| | Uniquely | | | | | | | | | | | | | | |
| L1M4b_dup400 | Uniquely | No | — | — | LINE | Other L1 | chr1: 240390155-240391321 | + | 85 | 1167 | FMN2 | Proximal | 0 | Not documented | 1 |
| MIRc_dup10291 | Multi | No | — | — | SINE | MIR | chr1: 241667256-241667372 | + | 130 | 117 | FH | Proximal | 0 | Not documented | 2 |
| AluSx_dup12061 | Uniquely | No | — | — | SINE | Alu | chr1: 242158939-242159233 | + | 49 | 295 | MAP1LC3C | Proximal | 0 | Not documented | 3 |
| CR1_Mam_dup13 | Multi | No | — | — | LINE | CR1 | chr1: 24294042-24294379 | − | 140 | 338 | SRSF10 | Proximal | 0 | Not documented | 4 |
| AluSg_dup594 | Multi | No | — | — | SINE | Alu | chr1: 24296854-24297148 | − | 34 | 295 | SRSF10 | Proximal | 0 | Not documented | 5 |
| L1PA5_dup724 | Uniquely | No | — | — | LINE | L1PA\|B\|x | chr1: 243970350-243976492 | + | 25 | 6143 | AKT3 | Proximal | 0 | EVE annotated | 6 |
| AluSx_dup12227 | Multi | No | — | — | SINE | Alu | chr1: 245007779-245008081 | + | 44 | 303 | COX20 | Proximal | 0 | Not documented | 7 |
| MER2_dup690 | Multi | No | — | — | DNA | DNA | chr1: 245008225-245008483 | − | 95 | 259 | COX20 | Proximal | 0 | Not documented | 8 |
| AluJo_dup6206 | Uniquely | No | — | — | SINE | Alu | chr1: 245285770-245286064 | − | 65 | 295 | EFCAB2 | Proximal | 0 | Not documented | 9 |
| MER1B_dup396 | Uniquely | No | — | — | DNA | DNA | chr1: 246243459-246243848 | + | 59 | 390 | SMYD3 | Proximal | 0 | Not documented | 1210 |
| L1PBa_dup124 | Uniquely | No | — | — | LINE | L1PA\|B\|x | chr1: 247214582-247216653 | − | 52 | 2072 | ZNF670 | Proximal | 0 | Not documented | 1 |
| L1PA14_dup233 | Uniquely | No | — | — | LINE | L1PA\|B\|x | chr1: 247325271-247325749 | + | 55 | 479 | ZNF124 | Proximal | 0 | Not documented | 2 |
| AluSx3_dup2511 | Multi | No | — | — | SINE | Alu | chr1: 247461629-247461916 | − | 87 | 288 | ZNF496 | Proximal | 0 | Not documented | 3 |
| MLT1C_dup1274 | Multi | No | — | — | LTR | ERVL- | chr1: 247462420-247462819 | + | 83 | 400 | ZNF496 | Proximal | 0 | Not documented | 4 |
| | | | | | | MaLR | | | | | | | | | |
| HERVP71A-int_dup29 | Uniquely | No | — | — | LTR | ERV1 | chr1: 247520740-247523416 | + | 41 | 2677 | ZNF496 | Distal | 25593 | Not documented | 5 |
| MER31-int_dup175 | Multi | No | — | — | LTR | ERV1 | chr1: 249231334-249231780 | − | 99 | 447 | PGBD2 | Distal | 17190 | Not documented | 6 |
| AluSc_dup390 | Multi | No | — | — | SINE | Alu | chr1: 25073745-25074050 | + | 31 | 30€ | CLIC4 | Proximal | 0 | Not documented | 7 |
| L2b_dup1427 | Uniquely | No | — | — | LINE | L2 | chr1: 25088921-25089414 | − | 130 | 494 | CLIC4 | Proximal | 0 | Not documented | 8 |
| AluSz6_dup633 | Multi | No | — | — | SINE | Alu | chr1: 25092983-25093296 | − | 62 | 314 | CLIC4 | Proximal | 0 | Not documented | 9 |
| MER30_dup44 | Multi | No | — | — | DNA | DNA | chr1: 25093427-25093665 | − | 48 | 239 | CLIC4 | Proximal | 0 | Not documented | 1220 |
| AluJb_dup2481 | Multi/ | No | — | — | SINE | Alu | chr1: 25105679-25105977 | + | 69 | 299 | CLIC4 | Proximal | 0 | Not documented | 1 |
| | Uniquely | | | | | | | | | | | | | | |
| L2_dup411 | Multi/ | No | — | — | LINE | L2 | chr1: 25106177-25106497 | + | 160 | 321 | CLIC4 | Proximal | 0 | Not documented | 2 |
| | Uniquely | | | | | | | | | | | | | | |
| MIR_dup2340 | Multi/ | No | — | — | SINE | MIR | chr1: 25106589-25106817 | − | 120 | 229 | CLIC4 | Proximal | 0 | Not documented | 3 |
| | Uniquely | | | | | | | | | | | | | | |
| FRAM_dup118 | Multi | No | — | — | SINE | Alu | chr1: 25570551-25570607 | − | 16 | 57 | C1orf63 | Proximal | 0 | Not documented | 4 |
| AluJb_dup2514 | Multi | No | — | — | SINE | Alu | chr1: 25571188-25571451 | − | 70 | 264 | C1orf63 | Proximal | 0 | Not documented | 5 |
| MIRb_dup3603 | Multi | No | — | — | SINE | MIR | chr1: 26112112-26112249 | + | 130 | 138 | MAN1C1 | Proximal | 0 | Not documented | 6 |
| AluSx_dup2556 | Multi | No | — | — | SINE | Alu | chr1: 26143705-26144005 | − | 47 | 301 | RP1-317E23.6 | Proximal | 0 | Not documented | 7 |
| MIRb_dup3608 | Multi/ | No | — | — | SINE | MIR | chr1: 26144043-26144201 | − | 100 | 159 | RP1-317E23.6 | Proximal | 0 | Not documented | 8 |
| | Uniquely | | | | | | | | | | | | | | |
| L4_dup98 | Multi | No | — | — | LINE | RTE | chr1: 26802146-26802211 | + | 140 | 66 | HMGN2 | Proximal | 0 | Not documented | 9 |
| MIR3_dup1473 | Multi/ | No | — | — | SINE | MIR | chr1: 27272949-27273036 | + | 98 | 88 | NUDC | Proximal | 0 | Not documented | 1230 |
| | Uniquely | | | | | | | | | | | | | | |
| AluSx_dup2745 | Multi | No | — | — | SINE | Alu | chr1: 27273061-27273361 | + | 47 | 301 | NUDC | Proximal | 0 | Not documented | 1 |
| FAM_dup67 | Multi/ | No | — | — | SINE | Alu | chr1: 27992660-27992823 | + | 69 | 164 | IFIE | Proximal | 0 | Not documented | 2 |
| | Uniquely | | | | | | | | | | | | | | |
| LTR29_dup7 | Multi | No | — | — | LTR | ERV1 | chr1: 28148961-28149017 | − | 88 | 57 | STX12 | Proximal | 0 | Not documented | 3 |
| AluJo_dup1223 | Multi | No | — | — | SINE | Alu | chr1: 28150445-28150575 | + | 70 | 131 | STX12 | Proximal | 0 | Not documented | 4 |
| LTR16D1_dup7 | Multi | No | — | — | LTR | ERVL | chr1: 28562714-28562777 | − | 87 | 64 | ATPIF1 | Proximal | 0 | Not documented | 5 |
| MIR3_dup1581 | Multi | No | — | — | SINE | MIR | chr1: 28563768-28563825 | − | 110 | 58 | ATPIF1 | Proximal | 0 | Not documented | 6 |
| AluSx_dup3037 | Multi | No | — | — | SINE | Alu | chr1: 28835504-28835578 | − | 42 | 75 | RCC1 | Proximal | 0 | Not documented | 7 |
| MIRb_dup4921 | Multi/ | No | — | — | SINE | MIR | chr1: 32262035-32262195 | − | 120 | 161 | SPOCD1 | Proximal | 0 | Not documented | 8 |
| | Uniquely | | | | | | | | | | | | | | |
| MER117_dup67 | Multi | No | — | — | DNA | DNA | chr1: 32662457-32662521 | − | 110 | 65 | TXLNA | Proximal | 0 | Not documented | 9 |
| AluSp_dup1101 | Multi | No | — | — | SINE | Alu | chr1: 32696813-32697110 | + | 53 | 298 | EIF31 | Proximal | 0 | Not documented | 1240 |
| AluY_dup2188 | Multi | No | — | — | SINE | Alu | chr1: 33086720-33087026 | + | 41 | 307 | ZBTB8OS | Proximal | 0 | Not documented | 1 |
| AluSx_dup3484 | Multi | No | — | — | SINE | Alu | chr1: 33147876-33148053 | + | 71 | 178 | SYNC | Proximal | 0 | Not documented | 2 |
| MIRb_dup5117 | Multi/ | No | — | — | SINE | MIR | chr1: 33497147-33497367 | + | 100 | 221 | AK2 | Proximal | 0 | Not documented | 3 |
| | Uniquely | | | | | | | | | | | | | | |
| MIRb_dup5505 | Multi | No | — | — | SINE | MIR | chr1: 35314664-35314828 | − | 120 | 165 | SMIM12 | Proximal | 0 | Not documented | 4 |
| L2c_dup2037 | Multi | No | — | — | LINE | L2 | chr1: 35314798-35315147 | − | 140 | 350 | SMIM12 | Proximal | 0 | Not documented | 5 |
| AluY_dup2305 | Multi | No | — | — | SINE | Alu | chr1: 35315302-35315614 | + | 28 | 313 | SMIM12 | Proximal | 0 | Not documented | 6 |
| AluJb_dup3690 | Multi | No | — | — | SINE | Alu | chr1: 36067653-36067955 | + | 70 | 303 | PSMB2 | Proximal | 0 | Not documented | 7 |
| MIR3_dup2089 | Multi | No | — | — | SINE | MIR | chr1: 36179615-36179701 | − | 110 | 87 | C1orf216 | Proximal | 0 | Not documented | 8 |
| MLT1L_dup116 | Multi | No | — | — | LTR | ERVL- | chr1: 36392879-36393269 | − | 120 | 391 | AG01 | Proximal | 0 | Not documented | 9 |
| | | | | | | MaLR | | | | | | | | | |
| MIRb_dup5588 | Multi/ | No | — | — | SINE | MIR | chr1: 36393363-36393480 | − | 120 | 118 | AGO1 | Proximal | 0 | Not documented | 1250 |
| | Uniquely | | | | | | | | | | | | | | |
| AluSg_dup72 | Multi | No | — | — | SINE | Alu | chr1: 3654142-3654439 | + | 41 | 298 | TP73 | Proximal | 1378 | Not documented | 1 |
| MIR_dup3659 | Multi | No | — | — | SINE | MIR | chr1: 36805887-36805963 | − | 62 | 77 | STK40 | Proximal | 0 | Not documented | 2 |
| AluSz6_dup1003 | Uniquely | No | — | — | SINE | Alu | chr1: 36915156-36915466 | + | 41 | 311 | OSCP1 | Proximal | 0 | Not documented | 3 |
| Tigger3d | Multi | No | — | — | DNA | DNA | chr1: 3696703-3696997 | − | 110 | 295 | LRRC47 | Proximal | 0 | Not documented | 4 |
| AluYc_dup11 | Multi | No | — | — | SINE | Alu | chr1: 3697223-3697269 | + | 39 | 47 | LRRC47 | Proximal | 0 | Not documented | 5 |
| AluSc_dup41 | Multi | No | — | — | SINE | Alu | chr1: 3697270-3697561 | + | 48 | 292 | LRRC47 | Proximal | 0 | Not documented | 6 |
| L3_dup614 | Uniquely | No | — | — | LINE | CR1 | chr1: 38022852-38022989 | + | 120 | 138 | DNALI1 | Proximal | 0 | Not documented | 7 |
| L2a_dup3054 | Multi/ | No | — | — | LINE | L2 | chr1: 38023518-38024318 | + | 120 | 801 | DNALI1 | Proximal | 0 | Not documented | 8 |
| | Uniquely | | | | | | | | | | | | | | |
| AluSq2_dup95 | Multi | No | — | — | SINE | Alu | chr1: 3805978-3806288 | + | 41 | 311 | C1orf174 | Proximal | 0 | Not documented | 9 |
| FLAM_C_dup546 | Multi | No | — | — | SINE | Alu | chr1: 38147616-38147738 | + | 89 | 123 | C1orf109 | Proximal | 0 | Not documented | 1260 |
| AluSx1_dup2544 | Multi/ | No | — | — | SINE | Alu | chr1: 38155688-38155956 | − | 20 | 269 | C1orf109 | Proximal | 0 | Not documented | 1 |
| | Uniquely | | | | | | | | | | | | | | |
| L1M5_dup721 | Multi | No | — | — | LINE | Other L1 | chr1: 38268463-38268683 | + | 150 | 221 | YRDC | Proximal | 0 | Not documented | 2 |
| FLAM_C_dup554 | Multi | No | — | — | SINE | Alu | chr1: 38269041-38269176 | − | 77 | 136 | YRDC | Proximal | 0 | Not documented | 3 |
| AluJo_dup1639 | Multi | No | — | — | SINE | Alu | chr1: 38423062-38423302 | − | 77 | 241 | SF3A3 | Proximal | 0 | Not documented | 4 |
| AluJr4_dup347 | Multi | No | — | — | SINE | Alu | chr1: 38489519-38489809 | − | 75 | 291 | UTP11L | Proximal | 0 | Not documented | 5 |
| AluYb8_dup38 | Multi | No | — | — | SINE | Alu | chr1: 38490067-38490378 | − | 2.7 | 312 | UTP11L | Proximal | 0 | Not documented | 6 |
| AluSx1_dup2616 | Multi/ | No | — | — | SINE | Alu | chr1: 39470696-39470995 | + | 40 | 300 | AKIRIN1 | Proximal | 0 | Not documented | 7 |
| | Uniquely | | | | | | | | | | | | | | |
| AluSp_dup1306 | Uniquely | No | — | — | SINE | Alu | chr1: 39506687-39506981 | − | 49 | 295 | NDUFS5 | Distal | 6380 | Not documented | 8 |
| AluYc_dup186 | Multi | No | — | — | SINE | Alu | chr1: 39574179-39574285 | − | 34 | 107 | MACF1 | Proximal | 0 | Not documented | 9 |
| MIRb_dup6134 | Multi | No | — | — | SINE | MIR | chr1: 39574725-39574903 | + | 130 | 179 | MACF1 | Proximal | 0 | Not documented | 1270 |
| AluSx3_dup716 | Multi | No | — | — | SINE | Alu | chr1: 39574936-39575246 | − | 57 | 311 | MACF1 | Proximal | 0 | Not documented | 1 |
| MIRb_dup6135 | Multi | No | — | — | SINE | MIR | chr1: 39575336-39575423 | − | 120 | 88 | MACF1 | Proximal | 0 | Not documented | 2 |
| AluJb_dup4016 | Multi/ | No | — | — | SINE | Alu | chr1: 39576886-39577038 | − | 110 | 153 | MACF1 | Proximal | 0 | Not documented | 3 |
| | Uniquely | | | | | | | | | | | | | | |
| MIR_dup3998 | Multi | No | — | — | SINE | MIR | chr1: 39590377-39590627 | − | 120 | 251 | MACF1 | Proximal | 0 | Not documented | 4 |
| AluSq2_dup1371 | Multi | No | — | — | SINE | Alu | chr1: 39756338-39756646 | − | 40 | 309 | MACF1 | Proximal | 0 | Not documented | 5 |
| AluSx1_dup2638 | Multi | No | — | — | SINE | Alu | chr1: 39756652-39756947 | − | 38 | 296 | MACF1 | Proximal | 0 | Not documented | 6 |
| L2a_dup3211 | Uniquely | No | — | — | LINE | L2 | chr1: 39785630-39786020 | + | 140 | 391 | MACF1 | Proximal | 0 | Not documented | 7 |
| FRAM_dup229 | Multi | No | — | — | SINE | Alu | chr1: 40345185-40345264 | + | 70 | 80 | TRIT1 | Proximal | 0 | Not documented | 8 |
| AluSq2_dup1394 | Multi/ | No | — | — | SINE | Alu | chr1: 40345265-40345566 | + | 51 | 302 | TRIT1 | Proximal | 0 | Not documented | 9 |
| | Uniquely | | | | | | | | | | | | | | |
| L1HS_dup7 | Multi | No | — | — | LINE | L1PA\|B\|x | chr1: 41673424-41674113 | + | 6.8 | 690 | SCMH1 | Proximal | 0 | Not documented | 1280 |
| L1PA4_dup103 | Uniquely | No | — | — | LINE | L1PA\|B\|x | chr1: 42699709-42701220 | − | 35 | 1512 | FOXJ3 | Proximal | 0 | EVE annotated | 1 |
| L2_dup906 | Uniquely | No | — | — | LINE | L2 | chr1: 43826912-43827388 | + | 150 | 477 | CDC20 | Proximal | 0 | Not documented | 2 |
| L1MB7_dup445 | Uniquely | No | — | — | LINE | Other L1 | chr1: 43827946-43828237 | + | 90 | 292 | CDC20 | Proximal | | Not documented | 3 |
| MIRb_dup6733 | Multi | No | — | — | SINE | MIR | chr1: 43850403-43850629 | − | 85 | 227 | MED8 | Proximal | 0 | Not documented | 4 |
| MER20_dup314 | Multi | No | — | — | DNA | DNA | chr1: 44438573-44438781 | − | 63 | 209 | DPH2 | Proximal | 0 | Not documented | 5 |
| MER3_dup210 | Multi | No | — | — | DNA | DNA | chr1: 44438794-44438978 | − | 66 | 185 | DPH2 | Proximal | 0 | Not documented | 6 |
| MIR3_dup2544 | Multi | No | — | — | SINE | MIR | chr1: 44456382-44456470 | + | 120 | 89 | B4GALT2 | Proximal | 0 | Not documented | 7 |
| AluJb_dup4632 | Uniquely | No | — | — | SINE | Alu | chr1: 45959843-45960153 | + | 72 | 311 | CCDC163P | Proximal | 0 | Not documented | 8 |
| AluY_dup3019 | Multi/ | No | — | — | SINE | Alu | chr1: 46077528-46077844 | − | 30 | 317 | NASP | Proximal | 0 | Not documented | 9 |
| | Uniquely | | | | | | | | | | | | | | |
| SVA_F_dup27 | Multi | No | — | — | Other | SVA | chr1: 46159588-46159704 | + | 68 | 117 | TMEM69 | Proximal | 0 | Not documented | 1290 |
| L1PB1_dup116 | Uniquely | No | — | — | LINE | L1PA\|B\|x | chr1: 50013744-50018503 | − | 60 | 4760 | AGBL4 | Proximal | 0 | EVE annotated | 1 |
| L1PA15_dup64 | Uniquely | No | — | — | LINE | L1PA\|B\|x | chr1: 51273296-51275645 | + | 83 | 2350 | FAF1 | Proximal | 0 | Not documented | 2 |
| L1MEc_dup389 | Uniquely | No | — | — | LINE | Other L1 | chr1: 52972117-52973217 | − | 100 | 1101 | ZCCHC11 | Proximal | 0 | Not documented | 3 |
| AluSz_dup3110 | Multi | No | — | — | SINE | Alu | chr1: 53679319-53679619 | + | 44 | 301 | CPT2 | Proximal | 0 | Not documented | 4 |
| L2c_dup3981 | Multi | No | — | — | LINE | L2 | chr1: 54320524-54320755 | − | 150 | 232 | YIPF1 | Proximal | 0 | Not documented | 5 |
| LTR33_dup1 | Uniquely | No | — | — | LTR | ERVL | chr1: 54394701-54395178 | + | 130 | 478 | HSPB11 | Proximal | 0 | Not documented | 6 |
| MIRc_dup3539 | Multi/ | No | — | — | SINE | MIR | chr1: 57025451-57025555 | + | 100 | 105 | PPAP2B | Proximal | 0 | Not documented | 7 |
| | Uniquely | | | | | | | | | | | | | | |
| L2b_dup3969 | Uniquely | No | — | — | LINE | L2 | chr1: 57025832-57025923 | + | 150 | 92 | PPAP2B | Proximal | | Not documented | 8 |
| L1HS_dup13 | Multi | No | — | — | LINE | L1PA\|B\|x | chr1: 58580619-58581671 | + | 3.2 | 1053 | DAB1 | Proximal | 0 | Not documented | 9 |
| L1M4_dup3 | Multi | No | — | — | LINE | Other L1 | chr1: 586903-587477 | + | 120 | 575 | OR4F16 | Distal | 33583 | Not documented | 1300 |
| L1ME1_dup681 | Uniquely | No | — | — | LINE | Other L1 | chr1: 61095249-61096758 | − | 98 | 1510 | NFIA | Distal | 234174 | Not documented | 1 |
| L1MEc_dup458 | Uniquely | No | — | — | LINE | Other L1 | chr1: 61096809-61097817 | − | 130 | 1009 | NFIA | Distal | 233115 | Not documented | 2 |
| MER89-int_dup7 | Uniquely | No | — | — | LTR | ERV1 | chr1: 61249232-61250904 | − | 110 | 1673 | NFIA | Distal | 80028 | Not documented | 3 |
| L1M2_dup109 | Uniquely | No | — | — | LINE | Other L1 | chr1: 61272316-61274076 | − | 97 | 1761 | NFIA | Distal | 56856 | Not documented | 4 |
| L1MA2_dup105 | Uniquely | No | — | — | LINE | Other L1 | chr1: 61291657-61292443 | − | 49 | 787 | NFIA | Distal | 38489 | Not documented | 5 |
| AluSq2_dup1865 | Uniquely | No | — | — | SINE | Alu | chr1: 61578983-61579293 | + | 59 | 311 | NFIA | Proximal | 0 | Not documented | 6 |
| AluSx1_dup3579 | Multi | No | — | — | SINE | Alu | chr1: 61583574-61583886 | + | 48 | 313 | NFIA | Proximal | 0 | Not documented | 7 |
| L3_dup1300 | Uniquely | No | — | — | LINE | CR1 | chr1: 61672589-61672738 | + | 160 | 150 | NFIA | Proximal | 0 | Not documented | 8 |
| Eulor5A | Uniquely | No | — | — | DNA | DNA | chr1: 61691834-61691945 | − | 170 | 112 | NFIA | Proximal | 0 | Not documented | 9 |
| L1PB1_dup152 | Uniquely | No | — | — | LINE | L1PA\|B\|x | chr1: 62009409-62013564 | − | 45 | 4156 | NFIA | Distal | 80945 | EVE annotated | 1310 |
| L2c_dup5247 | Uniquely | No | — | — | LINE | L2 | chr1: 63913064-63913324 | − | 150 | 261 | ITGB3BP | Proximal | 0 | Not documented | 1 |
| MIRb_dup10687 | Multi/ | No | — | — | SINE | MIR | chr1: 65694618-65694856 | − | 160 | 239 | AK4 | Proximal | 0 | Not documented | 2 |
| | Uniquely | | | | | | | | | | | | | | |
| AluJr_dup2494 | Multi/ | No | — | — | SINE | Alu | chr1: 65694952-65695230 | + | 91 | 279 | AK4 | Proximal | 0 | Not documented | 3 |
| | Uniquely | | | | | | | | | | | | | | |
| MIRb_dup10688 | Multi | No | — | — | SINE | MIR | chr1: 65696179-65696235 | − | 120 | 57 | AK4 | Proximal | 0 | Not documented | 4 |
| X6B_LINE_dup2 | Multi | No | — | — | LINE | CR1 | chr1: 65697106-65697146 | − | 80 | 41 | AK4 | Proximal | 0 | Not documented | 5 |
| MER5A1_dup346 | Multi | No | — | — | DNA | DNA | chr1: 65898008-65898156 | + | 98 | 149 | LEPROT | Proximal | 0 | Not documented | 6 |
| MIRc_dup4318 | Multi | No | — | — | SINE | MIR | chr1: 65898204-65898278 | + | 140 | 75 | LEPROT | Proximal | 0 | Not documented | 7 |
| L1MA7_dup125 | Multi | No | — | — | LINE | Other L1 | chr1: 65898354-65898633 | + | 70 | 280 | LEPROT | Proximal | 0 | Not documented | 8 |
| MIR3_dup4012 | Multi/ | No | — | — | SINE | MIR | chr1: 68167660-68167830 | − | 120 | 171 | GNG12 | Proximal | 0 | Not documented | 9 |
| | Uniquely | | | | | | | | | | | | | | |
| MER20B_dup86 | Multi/ | No | — | — | DNA | DNA | chr1: 68564237-68564733 | − | 95 | 497 | WLS | Proximal | 0 | Not documented | 1320 |
| | Uniquely | | | | | | | | | | | | | | |
| L2c_dup5663 | Uniquely | No | — | — | LINE | L2 | chr1: 68657605-68657822 | + | 150 | 218 | WLS | Proximal | 0 | Not documented | 1 |
| MER103C_dup13 | Multi | No | — | — | DNA | DNA | chr1: 6880241-6880269 | − | 31 | 29 | CAMTA1 | Proximal | 0 | Not documented | 2 |
| AluSz_dup3600 | Uniquely | No | — | — | SINE | Alu | chr1: 68832773-68833078 | + | 64 | 306 | RPE65 | Distal | 61428 | Not documented | 3 |
| MER41B_dup66 | Multi/ | No | — | — | LTR | ERV1 | chr1: 70832131-70832544 | − | 44 | 414 | HHLA3 | Proximal | 0 | Not documented | 4 |
| | Uniquely | | | | | | | | | | | | | | |
| LTR7_dup46 | Multi/ | No | — | — | LTR | ERV1 | chr1: 70833312-70833744 | + | 55 | 433 | HHLA3 | Proximal | 0 | Not documented | 5 |
| | Uniquely | | | | | | | | | | | | | | |
| Plat_L3_dup94 | Multi/ | No | — | — | LINE | CR1 | chr1: 71531296-71531491 | + | 160 | 196 | ZRANB2 | Proximal | 0 | Not documented | 6 |
| | Uniquely | | | | | | | | | | | | | | |
| FRAM_dup339 | Multi | No | — | — | SINE | Alu | chr1: 71534481-71534635 | − | 91 | 155 | ZRANB2 | Proximal | 0 | Not documented | 7 |
| L1HS_dup27 | Multi | No | — | — | LINE | L1PA\|B\|x | chr1: 74984566-74985255 | − | 2.7 | 690 | FPGT-TNNI3K | Proximal | 0 | Not documented | 8 |
| L1PB2_dup59 | Uniquely | No | — | — | LINE | L1PA\|B\|x | chr1: 75748613-75753599 | − | 49 | 4987 | SLC44A5 | Proximal | 0 | Not documented | 9 |
| L1PA16_dup223 | Uniquely | No | — | — | LINE | L1PA\|B\|x | chr1: 76068667-76070775 | − | 53 | 2109 | SLC44A5 | Proximal | 0 | Not documented | 1330 |
| L1MA9_dup217 | Uniquely | No | — | — | LINE | Other L1 | chr1: 78073622-78078745 | + | 70 | 5124 | ZZZ3 | Proximal | 0 | Not documented | 1 |
| AluSx1_dup4008 | Multi | No | — | — | SINE | Alu | chr1: 78162486-78162775 | + | 39 | 290 | USP33 | Proximal | 0 | Not documented | 2 |
| HAL1b_dup110 | Uniquely | No | — | — | LINE | Other L1 | chr1: 79102018-79102430 | + | 130 | 413 | IFI44L | Proximal | 0 | Not documented | 3 |
| L2c_dup6422 | Multi | No | — | — | LINE | L2 | chr1: 79107759-79107933 | + | 150 | 175 | IFI44L | Proximal | 0 | Not documented | 4 |
| MIRb_dup11984 | Multi | No | — | — | SINE | MIR | chr1: 79107912-79107953 | + | 110 | 42 | IFI44L | Proximal | 0 | Not documented | 5 |
| LTR26_dup32 | Multi | No | — | — | LTR | ERV1 | chr1: 79109041-79109628 | − | 130 | 588 | IFI44L | Proximal | 0 | Not documented | 6 |
| LTR23_dup23 | Multi | No | — | — | LTR | ERV1 | chr1: 79110638-79111069 | + | 71 | 432 | IFI44L | Proximal | 0 | Not documented | 7 |
| MLT1J_dup452 | Multi | No | — | — | LTR | ERVL- | chr1: 79121448-79121562 | − | 120 | 115 | IFI44 | Proximal | 0 | Not documented | 8 |
| | | | | | | MaLR | | | | | | | | | |
| L1PA15_dup132 | Multi | No | — | — | LINE | L1PA\|B\|x | chr1: 83601482-83605836 | − | 66 | 4355 | TTLL7 | Distal | 724876 | Not documented | 9 |
| AluSx1_dup4115 | Multi | No | — | — | SINE | Alu | chr1: 83645619-83645943 | + | 53 | 325 | TTLL7 | Distal | 684769 | Not documented | 1340 |
| MIRb_dup12470 | Multi | No | — | — | SINE | MIR | chr1: 85717573-85717787 | + | 130 | 215 | C1orf52 | Proximal | 0 | Not documented | 1 |
| AluJr_dup2794 | Multi | No | — | — | SINE | Alu | chr1: 86119168-86119440 | + | 87 | 273 | ZNHIT6 | Proximal | 0 | Not documented | 2 |
| AluSz_dup3951 | Multi | No | — | — | SINE | Alu | chr1: 86852048-86852370 | − | 56 | 323 | ODF2L | Proximal | 0 | Not documented | 3 |
| MIRb_dup12602 | Uniquely | No | — | — | SINE | MIR | chr1: 87210790-87211019 | + | 150 | 230 | SH3GLB1 | Proximal | 0 | Not documented | 4 |
| AluSx_dup6134 | Uniquely | No | — | — | SINE | Alu | chr1: 87212270-87212565 | + | 52 | 296 | SH3GLB1 | Proximal | 0 | Not documented | 5 |
| L1ME4a_dup1456 | Multi | No | — | — | LINE | Other L1 | chr1: 87213558-87214032 | − | 100 | 475 | SH3GLB1 | Proximal | 0 | Not documented | 6 |
| L1MC2_dup154 | Uniquely | No | — | — | LINE | Other L1 | chr1: 87425355-87426807 | + | 74 | 1453 | HS2ST1 | Proximal | 0 | Not documented | 7 |
| MIRc_dup5135 | Multi | No | — | — | SINE | MIR | chr1: 87571536-87571789 | + | 70 | 254 | RP5-105215.2 | Proximal | 0 | Not documented | 8 |
| MIR_dup8129 | Multi | No | — | — | SINE | MIR | chr1: 89401472-89401611 | − | 120 | 140 | CCBL2 | Proximal | 0 | Not documented | 9 |
| L2_dup2069 | Uniquely | No | — | — | LINE | L2 | chr1: 89511538-89512187 | − | 140 | 650 | GBP1 | Distal | 5816 | Not documented | 1350 |
| MER58A_dup404 | Multi/ | No | — | — | DNA | DNA | chr1: 89523962-89524176 | − | 100 | 215 | GBP1 | Proximal | 0 | Not documented | 1 |
| | Uniquely | | | | | | | | | | | | | | |
| L4_dup19 | Multi | No | — | — | LINE | RTE | chr1: 8954345-8954410 | + | 140 | 66 | ENO1 | Distal | 15038 | Not documented | 2 |
| MSTA_dup519 | Uniquely | No | — | — | LTR | ERVL- | chr1: 91254907-91255249 | − | 52 | 343 | BARHL2 | Distal | 72114 | Not documented | 3 |
| | | | | | | MaLR | | | | | | | | | |
| L3_dup1895 | Multi | No | — | — | LINE | CR1 | chr1: 91599982-91600040 | − | 120 | 59 | ZNF644 | Distal | 112154 | Not documented | 4 |
| Tigger7_dup90 | Multi | No | — | — | DNA | DNA | chr1: 91751235-91751659 | + | 73 | 425 | HFM1 | Proximal | 0 | Not documented | 5 |
| MLT1B_dup548 | Multi | No | — | — | LTR | ERVL- | chr1: 91760820-91761193 | − | 69 | 374 | HFM1 | Proximal | 0 | Not documented | 6 |
| | | | | | | MaLR | | | | | | | | | |
| L1MA9_dup303 | Multi | No | — | — | LINE | Other L1 | chr1: 91822026-91823659 | + | 83 | 1634 | HFM1 | Proximal | 0 | Not documented | 7 |
| L1HS_dup39 | Multi | No | — | — | LINE | L1PA\|B\|x | chr1: 91853148-91853486 | + | 5.5 | 339 | HFM1 | Proximal | 0 | Not documented | 8 |
| ERVL-B4-int_dup8 | Uniquely | No | — | — | LTR | ERVL | chr1: 9357527-9358428 | + | 72 | 902 | SPSB1 | Proximal | 0 | Not documented | 9 |
| L2b_dup5634 | Multi/ | No | — | — | LINE | L2 | chr1: 93804558-93804875 | + | 160 | 318 | DR1 | Distal | 6571 | Not documented | 1360 |
| | Uniquely | | | | | | | | | | | | | | |
| AluSg_dup1749 | Multi | No | — | — | SINE | Alu | chr1: 94353030-94353321 | − | 30 | 292 | GCLM | Proximal | 0 | Not documented | 1 |
| AluSg7_dup353 | Multi/ | No | — | — | SINE | Alu | chr1: 94995348-94995653 | + | 39 | 30€ | F3 | Proximal | 0 | Not documented | 2 |
| | Uniquely | | | | | | | | | | | | | | |
| AluSz_dup4190 | Uniquely | No | — | — | SINE | Alu | chr1: 94996846-94997156 | − | 61 | 311 | F3 | Proximal | 0 | Not documented | 3 |
| MIR3_dup4679 | Uniquely | No | — | — | SINE | MIR | chr1: 94998363-94998542 | − | 140 | 180 | F3 | Proximal | 0 | Not documented | 4 |
| AluSx1_dup4432 | Multi | No | — | — | SINE | Alu | chr1: 94999408-94999722 | − | 50 | 315 | F3 | Proximal | 0 | Not documented | 5 |
| Tigger2_dup106 | Multi | No | — | — | DNA | DNA | chr1: 94999723-94999975 | + | 56 | 253 | F3 | Proximal | 0 | Not documented | 6 |
| L1M5_dup1914 | Multi | No | — | — | LINE | Other L1 | chr1: 95368070-95368150 | − | 100 | 81 | CNN3 | Proximal | 0 | Not documented | 7 |
| L2c_dup7507 | Uniquely | No | — | — | LINE | L2 | chr1: 95378141-95378228 | + | 95 | 88 | CNN3 | Proximal | 0 | Not documented | 8 |
| L3_dup1961 | Uniquely | No | — | — | LINE | CR1 | chr1: 95379034-95379387 | + | 130 | 354 | CNN3 | Proximal | 0 | Not documented | 9 |
| L3_dup2023 | Multi | No | — | — | LINE | CR1 | chr1: 98012273-98012357 | − | 120 | 85 | DPYD | Proximal | 0 | Not documented | 1370 |
| HERVH-int_dup4175 | Multi | No | — | — | LTR | ERV1 | chr10: 102141063-102141511 | + | 93 | 449 | SCD | Distal | 16473 | Not documented | 1 |
| HERVH-int_dup4177 | Uniquely | No | — | — | LTR | ERV1 | chr10: 102143311-102144762 | + | 58 | 1452 | SCD | Distal | 18721 | EVE annotated | 2 |
| LTR7B_dup537 | Multi | No | — | — | LTR | ERV1 | chr10: 102147801-102148155 | + | 27 | 355 | SCD | Distal | 23211 | Not documented | 3 |
| L2b_dup59840 | Multi | No | — | — | LINE | L2 | chr10: 102741112-102741167 | + | 110 | 56 | SEMA4G | Proximal | 0 | Not documented | 4 |
| AluSg4_dup4385 | Multi | No | — | — | SINE | Alu | chr10: 104622811-104623121 | + | 20 | 311 | C10orf32 | Proximal | 0 | Not documented | 5 |
| AluJb_dup78536 | Uniquely | No | — | — | SINE | Alu | chr10: 11100761-11101045 | − | 100 | 285 | CELF2 | Proximal | 0 | Not documented | 6 |
| MIR_dup104161 | Uniquely | No | — | — | SINE | MIR | chr10: 11101046-11101151 | − | 130 | 106 | CELF2 | Proximal | 0 | Not documented | 7 |
| AluSx1_dup63931 | Multi/ | No | — | — | SINE | Alu | chr10: 115673602-115673909 | − | 50 | 308 | NHLRC2 | Proximal | 0 | Not documented | 8 |
| | Uniquely | | | | | | | | | | | | | | |
| AluSq2_dup32699 | Multi/ | No | — | — | SINE | Alu | chr10: 115673923-115674225 | − | 52 | 303 | NHLRC2 | Proximal | 0 | Not documented | 9 |
| | Uniquely | | | | | | | | | | | | | | |
| MIRb_dup141237 | Uniquely | No | — | — | SINE | MIR | chr10: 116238734-116238993 | + | 140 | 260 | ABLIM1 | Proximal | 0 | Not documented | 1380 |
| L1MEc_dup12600 | Uniquely | No | — | — | LINE | Other L1 | chr10: 127530517-127531332 | + | 97 | 816 | BCCIP | Proximal | 0 | Not documented | 1 |
| L1MB1_dup4168 | Uniquely | No | — | — | LINE | Other L1 | chr10: 129797329-129797503 | + | 91 | 175 | PTPRE | Proximal | 0 | Not documented | 2 |
| AluSz_dup53793 | Uniquely | No | — | — | SINE | Alu | chr10: 14603781-14604084 | − | 50 | 304 | FAM107B | Proximal | 0 | Not documented | |
| AluJb_dup78822 | Uniquely | No | — | — | SINE | Alu | chr10: 15144194-15144484 | − | 80 | 291 | RPP38 | Proximal | 0 | Not documented | 4 |
| MIRb_dup133330 | Multi | No | — | — | SINE | MIR | chr10: 17277426-17277624 | − | 140 | 199 | VIM | Proximal | 0 | Not documented | 5 |
| AluSp_dup27432 | Multi | No | — | — | SINE | Alu | chr10: 28811915-28812209 | − | 48 | 295 | WAC | Distal | 9214 | Not documented | |
| AluSq2_dup31103 | Multi | No | — | — | SINE | Alu | chr10: 31616024-31616338 | − | 55 | 315 | ZEB1 | Proximal | 0 | Not documented | 7 |
| MIR_dup105214 | Multi | No | — | — | SINE | MIR | chr10: 31617191-31617370 | + | 160 | 180 | ZEB1 | Proximal | 0 | Not documented | 8 |
| L1ME2_dup8012 | Multi | No | — | — | LINE | Other L1 | chr10: 31617956-31619126 | − | 91 | 1171 | ZEB1 | Proximal | 0 | Not documented | 9 |
| L1ME3E_dup3215 | Multi | No | — | — | LINE | Other L1 | chr10: 31622396-31623965 | + | 130 | 1570 | ZEB1 | Proximal | 0 | Not documented | 1390 |
| AluJr_dup43656 | Uniquely | No | — | — | SINE | Alu | chr10: 34598208-34598505 | + | 75 | 298 | PARD3 | Proximal | 0 | Not documented | 1 |
| L1MB2_dup5294 | Uniquely | No | — | — | LINE | Other L1 | chr10: 43039347-43041562 | + | 120 | 2216 | BMS1 | Distal | 236688 | Not documented | 2 |
| AluSx_dup79046 | Uniquely | No | — | — | SINE | Alu | chr10: 43949485-43949798 | − | 55 | 314 | ZNF487 | Proximal | 0 | Not documented | 3 |
| MIR_dup105609 | Multi/ | No | — | — | SINE | MIR | chr10: 45472527-45472642 | − | 85 | 116 | C10orf10 | Proximal | 0 | Not documented | 4 |
| | Uniquely | | | | | | | | | | | | | | |
| L4_dup11133 | Multi | No | — | — | LINE | RTE | chr10: 46118047-46118112 | + | 140 | 66 | ZFAND4 | Proximal | 0 | Not documented | 5 |
| MER39B_dup785 | Multi/ | No | — | — | LTR | ERV1 | chr10: 60933055-60933689 | − | 65 | 635 | PHYHIPL | Distal | 2662 | Not documented | 6 |
| | Uniquely | | | | | | | | | | | | | | |
| LTR81B_dup816 | Multi/ | No | — | — | LTR | Gypsy | chr10: 60933826-60934359 | − | 130 | 534 | PHYHIPL | Proximal | 1992 | Not documented | 7 |
| | Uniquely | | | | | | | | | | | | | | |
| L2a_dup105980 | Multi/ | No | — | — | LINE | L2 | chr10: 60935539-60935637 | + | 120 | 99 | PHYHIPL | Proximal | 714 | Not documented | 8 |
| | Uniquely | | | | | | | | | | | | | | |
| AluY_dup69916 | Uniquely | No | — | — | SINE | Alu | chr10: 69524853-69525156 | + | 14 | 304 | DNAJC12 | Distal | 31272 | Not documented | 9 |
| MER113_dup1853 | Uniquely | No | — | — | DNA | DNA | chr10: 71392988-71393213 | + | 130 | 226 | C10orf35 | Proximal | 0 | Not documented | 1400 |
| AluJo_dup40880 | Uniquely | No | — | — | SINE | Alu | chr10: 73772125-73772233 | + | 54 | 109 | CHST3 | Proximal | 0 | Not documented | 1 |
| AluY_dup70265 | Multi | No | — | — | SINE | Alu | chr10: 75009554-75009686 | + | 60 | 133 | MRPS16 | Proximal | 0 | Not documented | 2 |
| AluY_dup70266 | Multi | No | — | — | SINE | Alu | chr10: 75009687-75009992 | + | 25 | 306 | MRPS16 | Proximal | 0 | Not documented | 3 |
| L2b_dup58748 | Uniquely | No | — | — | LINE | L2 | chr10: 75012649-75012865 | − | 150 | 217 | MRPS16 | Proximal | 199 | Not documented | 4 |
| LTR37B_dup1328 | Multi | No | — | — | LTR | ERV1 | chr10: 79132632-79133047 | + | 75 | 416 | KCNMA1 | Proximal | 0 | Not documented | 5 |
| AluSx3_dup17200 | Uniquely | No | — | — | SINE | Alu | chr10: 80077197-80077501 | + | 45 | 305 | RPS24 | Distal | 260628 | Not documented | 6 |
| AluSx1_dup62704 | Multi | No | — | — | SINE | Alu | chr10: 82192115-82192412 | + | 56 | 298 | FAM213A | Proximal | 0 | Not documented | 7 |
| MLT1B_dup11733 | Uniquely | No | — | — | LTR | ERVL- | chr10: 84120387-84120858 | − | 87 | 472 | NRG3 | Proximal | 0 | Not documented | 8 |
| | | | | | | MaLR | | | | | | | | | |
| L1PA8A_dup1792 | Uniquely | No | — | — | LINE | L1PA\|B\|x | chr10: 84192231-84195525 | − | 56 | 3295 | NRG3 | Proximal | 0 | EVE annotated | 9 |
| L1MC4_dup18687 | Multi | No | — | — | LINE | Other L1 | chr10: 88565518-88566319 | − | 110 | 802 | BMPR1A | Proximal | 0 | Not documented | 1410 |
| MLT1l_dup7161 | Uniquely | No | — | — | LTR | ERVL- | chr10: 88575501-88575880 | + | 130 | 380 | BMPR1A | Proximal | 0 | Not documented | 1 |
| | | | | | | MaLR | | | | | | | | | |
| HAL1_dup17776 | Uniquely | No | — | — | LINE | Other L1 | chr10: 88576191-88576652 | + | 150 | 462 | BMPR1A | Proximal | 0 | Not documented | 2 |
| Tigger14a_dup865 | Multi | No | — | — | DNA | DNA | chr10: 89513129-89513227 | − | 98 | 99 | ATAD1 | Proximal | 0 | Not documented | 3 |
| MSTB-int_dup585 | Uniquely | No | — | — | LTR | ERVL- | chr10: 95769208-95770797 | − | 79 | 1590 | PLCE1 | Proximal | 0 | Not documented | 4 |
| | | | | | | MaLR | | | | | | | | | |
| MER58B_dup4274 | Multi/ | No | — | — | DNA | DNA | chr10: 96087973-96088299 | − | 72 | 327 | NOC3L | Proximal | 0 | Not documented | 5 |
| | Uniquely | | | | | | | | | | | | | | |
| AluJr_dup45230 | Uniquely | No | — | — | SINE | Alu | chr10: 96366105-96366398 | − | 100 | 294 | HELLS | Proximal | 0 | Not documented | 6 |
| HAL1_dup17869 | Uniquely | No | — | — | LINE | Other L1 | chr10: 96366494-96366897 | − | 140 | 404 | HELLS | Proximal | 0 | Not documented | 7 |
| AluSx1_dup63182 | Multi | No | — | — | SINE | Alu | chr10: 97150388-97150703 | − | 43 | 316 | SORBS1 | Proximal | 0 | Not documented | 8 |
| L3_dup29679 | Uniquely | No | — | — | LINE | CR1 | chr10: 97237832-97238179 | + | 150 | 348 | SORBS1 | Proximal | 0 | Not documented | 9 |
| L3_dup29681 | Uniquely | No | — | — | LINE | CR1 | chr10: 97268330-97268459 | + | 150 | 130 | SORBS1 | Proximal | 0 | Not documented | 1420 |
| AluSx1_dup63190 | Uniquely | No | — | — | SINE | Alu | chr10: 97286900-97287187 | + | 54 | 288 | SORBS1 | Proximal | 0 | Not documented | 11 |
| MIR3_dup57601 | Uniquely | No | — | — | SINE | MIR | chr10: 97287440-97287543 | + | 180 | 104 | SORBS1 | Proximal | 0 | Not documented | 12 |
| L2b_dup59586 | Uniquely | No | — | — | LINE | L2 | chr10: 97287806-97287879 | − | 150 | 74 | SORBS1 | Proximal | 0 | Not documented | 13 |
| AluSx_dup81460 | Uniquely | No | — | — | SINE | Alu | chr10: 97288394-97288705 | + | 34 | 312 | SORBS1 | Proximal | 0 | Not documented | 14 |
| L1PA15-16_dup967 | Uniquely | No | — | — | LINE | L1PA\|B\|x | chr11: 10097452-10098673 | + | 150 | 1222 | SBF2 | Proximal | 0 | Not documented | 1425 |
| THE1A-int_dup1079 | Uniquely | No | — | — | LTR | ERVL- | chr11: 101997392-101998938 | + | 40 | 1547 | YAP1 | Proximal | 0 | Not documented | 26 |
| | | | | | | MaLR | | | | | | | | | |
| L3b_dup4697 | Multi | No | — | — | LINE | CR1 | chr11: 10534181-10534226 | − | 100 | 46 | RNF141 | Proximal | 0 | Not documented | 27 |
| MIRc_dup66831 | Multi | No | — | — | SINE | MIR | chr11: 10534612-10534661 | − | 100 | 50 | RNF141 | Proximal | 0 | Not documented | 28 |
| AluSx1_dup64991 | Multi | No | — | — | SINE | Alu | chr11: 10535225-10535521 | − | 50 | 297 | RNF141 | Proximal | 0 | Not documented | 29 |
| MER20_dup10710 | Multi | No | — | — | DNA | DNA | chr11: 105879774-105879968 | − | 74 | 195 | MSANTD4 | Proximal | 0 | Not documented | 30 |
| L1ME4a_dup30099 | Multi | No | — | — | LINE | Other L1 | chr11: 105967870-105967981 | − | 100 | 112 | AASDHPPT | Proximal | 0 | Not documented | 31 |
| MLT1L_dup8237 | Multi | No | — | — | LTR | ERVL- | chr11: 105968151-105968244 | − | 130 | 94 | AASDHPPT | Proximal | 0 | Not documented | 32 |
| | | | | | | MaLR | | | | | | | | | |
| MLT1L_dup8238 | Multi | No | — | — | LTR | ERVL- | chr11: 105968275-105968468 | − | 140 | 194 | AASDHPPT | Proximal | 0 | Not documented | 33 |
| | | | | | | MaLR | | | | | | | | | |
| L1MA5_dup3299 | Uniquely | No | — | — | LINE | Other L1 | chr11: 106721935-106726254 | − | 97 | 4320 | GUCY1A2 | Proximal | 0 | Not documented | 34 |
| AluJo_dup44641 | Multi | No | — | — | SINE | Alu | chr11: 107662318-107662452 | + | 71 | 135 | SLC35F2 | Proximal | 0 | Not documented | 35 |
| AluSz_dup61078 | Multi/ | No | — | — | SINE | Alu | chr11: 107662455-107662717 | + | 64 | 263 | SLC35F2 | Proximal | 0 | Not documented | 36 |
| | Uniquely | | | | | | | | | | | | | | |
| Charlie14a_dup367 | Multi | No | — | — | DNA | DNA | chr11: 10876220-10876816 | − | 90 | 597 | ZBED5 | Proximal | 0 | Not documented | 37 |
| LTR50_dup1743 | Multi/ | No | — | — | LTR | ERVL | chr11: 10900242-10900865 | + | 110 | 624 | ZBED5 | Distal | 19900 | Not documented | 38 |
| | Uniquely | | | | | | | | | | | | | | |
| MIRc_dup71630 | Multi | No | — | — | SINE | MIR | chr11: 110333446-110333624 | − | 140 | 179 | FDX1 | Proximal | 0 | Not documented | 39 |
| MER21C_dup3625 | Uniquely | No | — | — | LTR | ERVL | chr11: 111512820-111513654 | − | 93 | 835 | SIK2 | Proximal | 0 | Not documented | 40 |
| MER5B_dup17299 | Uniquely | No | — | — | DNA | DNA | chr11: 111780589-111780795 | − | 63 | 207 | CRYAB | Proximal | 0 | Not documented | 41 |
| AluJr_dup48589 | Multi | No | — | — | SINE | Alu | chr11: 111892417-111892721 | + | 61 | 305 | DIXDC1 | Proximal | 0 | Not documented | 42 |
| MIRc_dup71699 | Uniquely | No | — | — | SINE | MIR | chr11: 111954850-111954954 | + | 130 | 105 | C11orf57 | Proximal | 0 | Not documented | 43 |
| MIRc_dup71700 | Multi/ | No | — | — | SINE | MIR | chr11: 111956926-111957088 | − | 140 | 163 | TIMM8B | Proximal | 0 | Not documented | 44 |
| | Uniquely | | | | | | | | | | | | | | |
| L2b_dup65900 | Uniquely | No | — | — | LINE | L2 | chr11: 114177915-114178848 | + | 150 | 934 | NNMT | Proximal | 0 | Not documented | 45 |
| L1MC1_dup9596 | Uniquely | No | — | — | LINE | Other L1 | chr11: 114204012-114207767 | + | 64 | 3756 | NNMT | Distal | 20006 | Not documented | 46 |
| Kanga2_a_dup1064 | Multi | No | — | — | DNA | DNA | chr11: 115238541-115239327 | − | 110 | 787 | CADM1 | Proximal | 0 | Not documented | 47 |
| MER3_dup7316 | Multi | No | — | — | DNA | DNA | chr11: 115239484-115239676 | + | 88 | 193 | CADM1 | Proximal | 0 | Not documented | 48 |
| MIRb_dup155163 | Multi | No | — | — | SINE | MIR | chr11: 115269728-115269930 | − | 150 | 203 | CADM1 | Proximal | 0 | Not documented | 49 |
| L2c_dup100431 | Uniquely | No | — | — | LINE | L2 | chr11: 116899013-116899504 | − | 150 | 492 | SIK3 | Proximal | 0 | Not documented | 1450 |
| AluSq2_dup34930 | Multi | No | — | — | SINE | Alu | chr11: 117039504-117039806 | − | 50 | 303 | PAFAH1B2 | Proximal | 0 | Not documented | 51 |
| AluSx3_dup18709 | Multi | No | — | — | SINE | Alu | chr11: 118471792-118472075 | − | 46 | 284 | ARCN1 | Proximal | 0 | Not documented | 52 |
| MIRc_dup66903 | Multi | No | — | — | SINE | MIR | chr11: 11985242-11985466 | + | 120 | 225 | DKK3 | Proximal | 0 | Not documented | 53 |
| AluSz_dup61616 | Uniquely | No | — | — | SINE | Alu | chr11: 120239827-120240137 | − | 60 | 311 | ARHGEF12 | Proximal | 0 | Not documented | 54 |
| MER82_dup2240 | Uniquely | No | — | — | DNA | DNA | chr11: 120903027-120903675 | + | 97 | 649 | TBCEL | Proximal | 0 | Not documented | 55 |
| L2b_dup66388 | Uniquely | No | — | — | LINE | L2 | chr11: 121958957-121959630 | − | 150 | 674 | BLID | Distal | 26433 | Not documented | 56 |
| MIR3_dup63889 | Multi/ | No | — | — | SINE | MIR | chr11: 121960691-121960861 | + | 170 | 171 | BLID | Distal | 25202 | Not documented | 57 |
| | Uniquely | | | | | | | | | | | | | | |
| MIRb_dup156043 | Multi | No | — | — | SINE | MIR | chr11: 121961208-121961355 | + | 84 | 148 | BLID | Distal | 24708 | Not documented | 58 |
| LTR33_dup6516 | Uniquely | No | — | — | LTR | ERVL | chr11: 122009760-122010247 | − | 140 | 488 | BLID | Dista | 22838 | Not documented | 59 |
| L1ME3A_dup11021 | Multi | No | — | — | LINE | Other L1 | chr11: 123995281-123995990 | − | 110 | 710 | VWA5A | Proximal | 0 | Not documented | 60 |
| MIR_dup120353 | Multi | No | — | — | SINE | MIR | chr11: 124493251-124493410 | + | 120 | 160 | TBRG1 | Proximal | 0 | Not documented | 61 |
| MIRb_dup156328 | Multi | No | — | — | SINE | MIR | chr11: 124493437-124493566 | − | 110 | 130 | TBRG1 | Proximal | 0 | Not documented | 62 |
| L3_dup32622 | Multi | No | — | — | LINE | CR1 | chr11: 124506474-124506515 | + | 110 | 42 | SIAE | Proximal | 0 | Not documented | 63 |
| L2b_dup66473 | Multi | No | — | — | LINE | L2 | chr11: 124789141-124789234 | − | 110 | 94 | HEPACAM | Proximal | 0 | Not documented | 64 |
| MER44A_dup1701 | Multi | No | — | — | DNA | DNA | chr11: 125357998-125358290 | + | 66 | 293 | FEZ1 | Proximal | 0 | Not documented | 65 |
| AluSg_dup26291 | Multi | No | — | — | SINE | Alu | chr11: 125358295-125358604 | + | 49 | 310 | FEZ1 | Proximal | 0 | Not documented | 66 |
| Charlie13a_dup422 | Multi | No | — | — | DNA | DNA | chr11: 125453238-125453416 | − | 120 | 179 | EI24 | Proximal | 0 | Not documented | 67 |
| L2a_dup118155 | Multi | No | — | — | LINE | L2 | chr11: 125491153-125491225 | + | 88 | 73 | STT3A | Proximal | 0 | Not documented | 68 |
| MER91A_dup2446 | Uniquely | No | — | — | DNA | DNA | chr11: 126654540-126654662 | − | 140 | 123 | KIRREL3 | Proximal | 0 | Not documented | 69 |
| AluSc_dup22616 | Uniquely | No | — | — | SINE | Alu | chr11: 126655491-126655797 | + | 45 | 307 | KIRREL3 | Proximal | 0 | Not documented | 70 |
| AluY_dup77341 | Multi/ | No | — | — | SINE | Alu | chr11: 126781600-126781902 | + | 21 | 303 | KIRREL3 | Proximal | | Not documented | 71 |
| | Uniquely | | | | | | | | | | | | | | |
| MER5A_dup24382 | Uniquely | No | — | — | DNA | DNA | chr11: 126811281-126811410 | + | 120 | 130 | KIRREL3 | Proximal | 0 | Not documented | 72 |
| Tigger1_dup8798 | Uniquely | No | — | — | DNA | DNA | chr11: 126830646-126833020 | + | 60 | 2375 | KIRREL3 | Proximal | 0 | Not documented | 73 |
| MIRb_dup143701 | Uniquely | No | — | — | SINE | MIR | chr11: 12741987-12742191 | − | 130 | 205 | TEAD1 | Proximal | 0 | Not documented | 74 |
| AluSx1_dup68863 | Multi/ | No | — | — | SINE | Alu | chr11: 129086295-129086588 | + | 53 | 294 | ARHGAP32 | Proximal | 0 | Not documented | 1475 |
| | Uniquely | | | | | | | | | | | | | | |
| MER33_dup6341 | Uniquely | No | — | — | DNA | DNA | chr11: 129092825-129093140 | − | 80 | 316 | ARHGAP32 | Proximal | 0 | Not documented | 76 |
| L1M4_dup12208 | Uniquely | No | — | — | LINE | Other L1 | chr11: 129094284-129094724 | + | 110 | 441 | ARHGAP32 | Proximal | 0 | Not documented | 77 |
| MIRb_dup143728 | Multi/ | No | — | — | SINE | MIR | chr11: 12959999-12960074 | − | 130 | 76 | TEAD1 | Proximal | 0 | Not documented | 78 |
| | Uniquely | | | | | | | | | | | | | | |
| MamRep564_dup516 | Multi/ | No | — | — | Other | Other | chr11: 12960331-12960455 | + | 120 | 125 | TEAD1 | Proximal | 0 | Not documented | 79 |
| | Uniquely | | | | | Repeats | | | | | | | | | |
| AluJr_dup46454 | Multi | No | — | — | SINE | Alu | chr11: 13344326-13344643 | − | 63 | 318 | ARNTL | Proximal | 0 | Not documented | 80 |
| L2c_dup93765 | Multi | No | — | — | LINE | L2 | chr11: 13344644-13344777 | − | 130 | 134 | ARNTL | Proximal | 0 | Not documented | 81 |
| L2a_dup110662 | Multi | No | — | — | LINE | L2 | chr11: 13344823-13345171 | + | 140 | 349 | ARNTL | Proximal | 0 | Not documented | 82 |
| L3_dup30520 | Multi | No | — | — | LINE | CR1 | chr11: 13375831-13375902 | + | 130 | 72 | ARNTL | Proximal | 0 | Not documented | 83 |
| L1MCa_dup5303 | Uniquely | No | — | — | LINE | Other L1 | chr11: 134003528-134004427 | + | 68 | 900 | JAM3 | Proximal | 0 | Not documented | 84 |
| L2a_dup118744 | Multi | No | — | — | LINE | L2 | chr11: 134118648-134118714 | − | 130 | 67 | THYN1 | Proximal | 0 | Not documented | 85 |
| AluSc8_dup13190 | Uniquely | No | — | — | SINE | Alu | chr11: 16328160-16328469 | − | 50 | 310 | SOX6 | Proximal | 0 | Not documented | 86 |
| Tigger1_dup8384 | Uniquely | No | — | — | DNA | DNA | chr11: 16495340-16497399 | − | 57 | 2060 | SOX6 | Proximal | 0 | Not documented | 87 |
| AluSq2_dup33317 | Multi | No | — | — | SINE | Alu | chr11: 17109796-17110088 | − | 34 | 293 | PIK3C2A | Proximal | 0 | Not documented | 88 |
| MIRb_dup144721 | Multi | No | — | — | SINE | MIR | chr11: 19799841-19799991 | + | 150 | 151 | NAV2 | Proximal | 0 | Not documented | 89 |
| MLT1G_dup1918 | Uniquely | No | — | — | LTR | ERVL- | chr11: 19987253-19987822 | − | 110 | 570 | NAV2 | Proximal | 0 | Not documented | 90 |
| | | | | | | MaLR | | | | | | | | | |
| L3_dup30724 | Uniquely | No | — | — | LINE | CR1 | chr11: 19989062-19989349 | + | 140 | 288 | NAV2 | Proximal | 0 | Not documented | 91 |
| AluSx3_dup17888 | Uniquely | No | — | — | SINE | Alu | chr11: 20074481-20074789 | + | 59 | 309 | NAV2 | Proximal | 0 | Not documented | 92 |
| AluY_dup73494 | Multi | No | — | — | SINE | Alu | chr11: 20096756-20097086 | − | 30 | 331 | NAV2 | Proximal | 0 | Not documented | 93 |
| L1PA7_dup9622 | Uniquely | No | — | — | LINE | L1PA\|B\|x | chr11: 24874912-24876629 | − | 47 | 1718 | LUZP2 | Proximal | 0 | Not documented | 94 |
| L1M2_dup6231 | Uniquely | No | — | — | LINE | Other L1 | chr11: 2669435-2671817 | − | 84 | 2383 | KCNQ1 | Proximal | 0 | Not documented | 95 |
| L3_dup30853 | Multi/ | No | — | — | LINE | CR1 | chr11: 27147196-27147302 | + | 150 | 107 | BBOX1 | Proximal | 0 | Not documented | 96 |
| | Uniquely | | | | | | | | | | | | | | |
| FRAM_dup5187 | Multi | No | — | — | SINE | Alu | chr11: 27517379-27517536 | + | 78 | 158 | LIN7C | Proximal | 0 | Not documented | 97 |
| Tigger3a_dup3649 | Uniquely | No | — | — | DNA | DNA | chr11: 33000425-33000725 | − | 51 | 301 | QSER1 | Proximal | 0 | Not documented | 98 |
| HAL1_dup18463 | Uniquely | No | — | — | LINE | Other L1 | chr11: 33163319-33163386 | − | 94 | 68 | CSTF3 | Proximal | 0 | Not documented | 99 |
| L1PB4_dup5263 | Multi | No | — | — | LINE | L1PA\|B\|x | chr11: 33911787-33911887 | + | 120 | 101 | LMO2 | Proximal | 0 | Not documented | 1500 |
| AluSq2_dup33509 | Multi/ | No | — | — | SINE | Alu | chr11: 33911893-33912198 | − | 56 | 306 | LMO2 | Proximal | 0 | Not documented | 1 |
| | Uniquely | | | | | | | | | | | | | | |
| MIRb_dup146238 | Multi/ | No | — | — | SINE | MIR | chr11: 33912290-33912397 | − | 110 | 108 | LMO2 | Proximal | 0 | Not documented | 2 |
| | Uniquely | | | | | | | | | | | | | | |
| MIRb_dup146239 | Multi/ | No | — | — | SINE | MIR | chr11: 33912566-33912669 | + | 140 | 104 | LMO2 | Proximal | 0 | Not documented | 3 |
| | Uniquely | | | | | | | | | | | | | | |
| SATR2_dup151 | Uniquely | No | — | — | Other | Other | chr11: 3408679-3411970 | + | 77 | 3292 | ZNF195 | Distal | 8232 | Not documented | 4 |
| | | | | | | Repeats | | | | | | | | | |
| L2a_dup112202 | Multi | No | — | — | LINE | L2 | chr11: 34120482-34120608 | + | 45 | 127 | CAPRIN1 | Proximal | 0 | Not documented | 5 |
| UCON23_dup21 | Multi | No | — | — | DNA | DNA | chr11: 34121588-34121621 | − | 54 | 34 | CAPRIN1 | Proximal | 0 | Not documented | 6 |
| L3_dup30983 | Multi | No | — | — | LINE | CR1 | chr11: 34474662-34474729 | + | 90 | 68 | CAT | Proximal | 0 | Not documented | 7 |
| HUERS-P3-int_dup461 | Uniquely | No | — | — | LTR | ERV1 | chr11: 34920716-34928687 | + | 64 | 7972 | APIP | Proximal | 0 | EVE annotated | 8 |
| MIR3_dup60360 | Multi | No | — | — | SINE | MIR | chr11: 35225799-35225914 | + | 170 | 116 | CD44 | Proximal | 0 | Not documented | 9 |
| MIR3_dup60361 | Multi | No | — | — | SINE | MIR | chr 11: 35247403-35247533 | + | 140 | 131 | CD44 | Proximal | 0 | Not documented | 10 |
| MIR_dup114155 | Multi | No | — | — | SINE | MIR | chr11: 35397336-35397558 | − | 130 | 223 | SLC1A2 | Proximal | 0 | Not documented | 11 |
| L1MC1_dup9239 | Uniquely | No | — | — | LINE | Other L1 | chr11: 35739963-35742454 | − | 63 | 2492 | TRIM44 | Proximal | 0 | Not documented | 12 |
| MIRb_dup146542 | Uniquely | No | — | — | SINE | MIR | chr11: 35808105-35808233 | + | 100 | 129 | TRIM44 | Proximal | 0 | Not documented | 13 |
| L1ME2_dup8641 | Uniquely | No | — | — | LINE | Other L1 | chr11: 35907455-35909593 | − | 120 | 2139 | LDLRAD3 | Distal | 55939 | Not documented | 14 |
| L1ME3A_dup10544 | Uniquely | No | — | — | LINE | Other L1 | chr11: 35926196-35927010 | + | 100 | 815 | LDLRAD3 | Distal | 38522 | Not documented | 15 |
| MLT1K_dup11265 | Multi/ | No | — | — | LTR | ERVL- | chr11: 35940035-35940544 | − | 150 | 510 | LDLRAD3 | Distal | 24988 | Not documented | 16 |
| | Uniquely | | | | | MaLR | | | | | | | | | |
| MIR3_dup59118 | Multi | No | — | — | SINE | MIR | chr11: 3847170-3847302 | + | 140 | 133 | PGAP2 | Proximal | 0 | Not documented | 17 |
| L1P3_dup2489 | Uniquely | No | — | — | LINE | L1PA\|B\|x | chr11: 40213725-40215995 | + | 47 | 2271 | LRRC4C | Proximal | 0 | Not documented | 18 |
| AluSz6_dup27597 | Multi | No | — | — | SINE | Alu | chr11: 4067290-4067449 | − | 43 | 160 | STIM1 | Proximal | 0 | Not documented | 19 |
| MIRb_dup147374 | Multi | No | — | — | SINE | MIR | chr11: 43902656-43902822 | − | 130 | 167 | ALKBH3 | Proximal | 0 | Not documented | 20 |
| AluSz_dup59061 | Multi | No | — | — | SINE | Alu | chr11: 45848036-45848380 | + | 82 | 345 | SLC35C1 | Distal | 13471 | Not documented | 21 |
| AluSc_dup21683 | Multi | No | — | — | SINE | Alu | chr11: 46699220-46699509 | + | 33 | 290 | ARHGAP1 | Proximal | 0 | Not documented | 22 |
| MIRb_dup148021 | Multi | No | — | — | SINE | MIR | chr11: 47442030-47442197 | + | 140 | 168 | PSMC3 | Proximal | 0 | Not documented | 23 |
| MER57B1_dup674 | Multi | No | — | — | LTR | ERV1 | chr11: 47639731-47639769 | − | 81 | 39 | MTCH2 | Proximal | 0 | Not documented | 24 |
| L2c_dup95979 | Multi | No | — | — | LINE | L2 | chr11: 57095345-57095535 | + | 170 | 191 | SSRP1 | Proximal | 0 | Not documented | 1525 |
| FLAM_C_dup13339 | Uniquely | No | — | — | SINE | Alu | chr11: 57509798-57509901 | + | 70 | 104 | C11orf31 | Proximal | 0 | Not documented | 26 |
| L1MEf_dup9441 | Multi | No | — | — | LINE | Other L1 | chr11: 57561511-57561552 | + | 65 | 42 | CTNND1 | Proximal | 0 | Not documented | 27 |
| L1PA2_dup3688 | Multi | No | — | — | LINE | L1PA\|B\|x | chr11: 57819104-57820079 | + | 5.9 | 976 | OR9Q1 | Proximal | 0 | Not documented | 28 |
| L1MEc_dup13024 | Uniquely | No | — | — | LINE | Other L1 | chr11: 58876187-58877073 | − | 120 | 887 | FAM111B | Proximal | 0 | Not documented | 29 |
| AluSx3_dup18160 | Multi/ | No | — | — | SINE | Alu | chr11: 58921177-58921484 | − | 49 | 308 | FAM111A | Proximal | 0 | Not documented | 30 |
| | Uniquely | | | | | | | | | | | | | | |
| L2a_dup113751 | Multi | No | — | — | LINE | L2 | chr11: 60618421-60618620 | − | 110 | 200 | PTGDR2 | Proximal | 0 | Not documented | 31 |
| MIRc_dup68944 | Uniquely | No | — | — | SINE | MIR | chr11: 61101332-61101425 | − | 92 | 94 | DAK | Proximal | 0 | Not documented | 32 |
| MIRb_dup148829 | Uniquely | No | — | — | SINE | MIR | chr11: 61101558-61101717 | + | 120 | 160 | DAK | Proximal | 0 | Not documented | 33 |
| MLT2B4_dup3263 | Uniquely | No | — | — | LTR | ERVL | chr11: 61240184-61240660 | + | 100 | 477 | RP11-286N22.8 | Proximal | 0 | Not documented | 34 |
| AluJb_dup87239 | Multi | No | — | — | SINE | Alu | chr11: 61240661-61240952 | − | 62 | 292 | RP11-286N22.8 | Proximal | 0 | Not documented | 35 |
| AluJb_dup87248 | Multi | No | — | — | SINE | Alu | chr11: 61567660-61567831 | − | 79 | 172 | FADS1 | Proximal | 0 | Not documented | 36 |
| AluSx_dup85555 | Multi | No | — | — | SINE | Alu | chr11: 61569355-61569516 | − | 34 | 162 | FADS1 | Proximal | 0 | Not documented | 37 |
| AluJb_dup87297 | Multi | No | — | — | SINE | Alu | chr11: 62159947-62160070 | + | 70 | 124 | ASRGL1 | Proximal | 0 | Not documented | 38 |
| MER5A_dup23652 | Multi | No | — | — | DNA | DNA | chr11: 62529937-62530088 | + | 120 | 152 | POLR2G | Proximal | 0 | Not documented | 39 |
| FLAM_C_dup13394 | Multi | No | — | — | SINE | Alu | chr11: 62557910-62558035 | − | 61 | 126 | TMEM223 | Proximal | 0 | Not documented | 40 |
| FLAM_C_dup13397 | Multi | No | — | — | SINE | Alu | chr11: 62619795-62619921 | + | 130 | 127 | SLC3A2 | Distal | 3598 | Not documented | 41 |
| AluSx3_dup18229 | Multi/ | No | — | — | SINE | Alu | chr11: 62649961-62650190 | − | 61 | 230 | SLC3A2 | Proximal | 0 | Not documented | 42 |
| | Uniquely | | | | | | | | | | | | | | |
| MIRb_dup148996 | Multi/ | No | — | — | SINE | MIR | chr11: 62651471-62651595 | + | 140 | 125 | SLC3A2 | Proximal | 0 | Not documented | 43 |
| | Uniquely | | | | | | | | | | | | | | |
| MER5A_dup23660 | Multi | No | — | — | DNA | DNA | chr11: 63392427-63392560 | + | 120 | 134 | ATL3 | Proximal | 0 | Not documented | 44 |
| AluSg_dup25440 | Multi | No | — | — | SINE | Alu | chr11: 63393187-63393472 | + | 59 | 286 | ATL3 | Proximal | 0 | Not documented | 45 |
| AluSx_dup85771 | Multi | No | — | — | SINE | Alu | chr11: 63393571-63393872 | + | 50 | 302 | ATL3 | Proximal | 0 | Not documented | 46 |
| MIR3_dup61368 | Multi/ | No | — | — | SINE | MIR | chr11: 63992557-63992647 | + | 98 | 91 | TRPT1 | Proximal | 0 | Not documented | 47 |
| | Uniquely | | | | | | | | | | | | | | |
| MIR3_dup61369 | Multi | No | — | — | SINE | MIR | chr11: 63993408-63993586 | − | 160 | 179 | TRPT1 | Proximal | 0 | Not documented | 48 |
| L2a_dup113953 | Multi | No | — | — | LINE | L2 | chr11: 64015359-64015491 | + | 150 | 133 | PPP1R14B | Proximal | 947 | Not documented | 49 |
| L2a_dup113954 | Multi | No | — | — | LINE | L2 | chr11: 64015529-64015612 | + | 110 | 84 | PPP1R14B | Proximal | 1117 | Not documented | 1550 |
| MIR_dup116121 | Multi/ | No | — | — | SINE | MIR | chr11: 64089073-64089204 | + | 130 | 132 | PRDX5 | Proximal | 0 | Not documented | 51 |
| | Uniquely | | | | | | | | | | | | | | |
| AluSc_dup21334 | Multi | No | — | — | SINE | Alu | chr11: 6413717-6414021 | − | 45 | 305 | SMPD1 | Proximal | 0 | Not documented | 52 |
| MIRc_dup69098 | Multi | No | — | — | SINE | MIR | chr11: 64574823-64575036 | − | 120 | 214 | MEN1 | Proximal | 0 | Not documented | 53 |
| AluSx1_dup66595 | Multi | No | — | — | SINE | Alu | chr11: 64893610-64893905 | − | 54 | 296 | MRPL49 | Proximal | 0 | Not documented | 54 |
| MIRc_dup69129 | Multi | No | — | — | SINE | MIR | chr11: 64894508-64894589 | + | 140 | 82 | MRPL49 | Proximal | 0 | Not documented | 55 |
| MIRb_dup143100 | Multi | No | — | — | SINE | MIR | chr11: 6503807-6503898 | − | 120 | 92 | TIMM10B | Proximal | 0 | Not documented | 56 |
| AluSx3_dup17768 | Multi | No | — | — | SINE | Alu | chr11: 6505320-6505621 | − | 36 | 302 | TIMM10B | Proximal | 0 | Not documented | 57 |
| L3_dup31302 | Multi | No | — | — | LINE | CR1 | chr11: 65422118-65422194 | − | 140 | 77 | RELA | Proximal | 0 | Not documented | 58 |
| MIRc_dup69158 | Multi | No | — | — | SINE | MIR | chr11: 65485534-65485590 | − | 140 | 57 | RNASEH2C | Proximal | 0 | Not documented | 59 |
| MIR_dup116219 | Multi/ | No | — | — | SINE | MIR | chr11: 65485903-65486002 | − | 130 | 100 | RNASEH2C | Proximal | 0 | Not documented | 60 |
| | Uniquely | | | | | | | | | | | | | | |
| MIRc_dup69163 | Multi | No | — | — | SINE | MIR | chr11: 65633864-65634031 | − | 140 | 168 | MUS81 | Proximal | 0 | Not documented | 61 |
| MIRc_dup69201 | Multi | No | — | — | SINE | MIR | chr11: 66113183-66113425 | − | 100 | 243 | B3GNT1 | Proximal | 0 | Not documented | 62 |
| AluJo_dup43748 | Multi | No | — | — | SINE | Alu | chr11: 66435170-66435467 | − | 53 | 298 | RBM4B | Proximal | 0 | Not documented | 63 |
| MIRc_dup69287 | Multi | No | — | — | SINE | MIR | chr11: 67275340-67275405 | − | 140 | 66 | CDK2AP2 | Proximal | 0 | Not documented | 64 |
| MIRb_dup149455 | Multi/ | No | — | — | SINE | MIR | chr11: 67375179-67375414 | + | 140 | 236 | NDUFV1 | Proximal | 0 | Not documented | 65 |
| | Uniquely | | | | | | | | | | | | | | |
| AluSz_dup59979 | Multi | No | — | — | SINE | Alu | chr11: 67375416-67375709 | + | 45 | 294 | NDUFV1 | Proximal | 0 | Not documented | 66 |
| MIRc_dup69305 | Multi | No | — | — | SINE | MIR | chr11: 67947582-67947681 | + | 82 | 100 | SUV420H1 | Proximal | 0 | Not documented | 67 |
| MIR_dup116581 | Multi | No | — | — | SINE | MIR | chr11: 70052964-70053034 | − | 120 | 71 | FADD | Proximal | 0 | Not documented | 68 |
| MIRb_dup149679 | Multi | No | — | — | SINE | MIR | chr11: 70053235-70053313 | + | 130 | 79 | FADD | Proximal | 0 | Not documented | 69 |
| L2a_dup114370 | Uniquely | No | — | — | LINE | L2 | chr11: 71159836-71159950 | − | 150 | 115 | DHCR7 | Proximal | 0 | Not documented | 70 |
| MIRb_dup149789 | Multi | No | — | — | SINE | MIR | chr11: 71822444-71822628 | + | 84 | 185 | ANAPC15 | Proximal | 0 | Not documented | 71 |
| L2b_dup63972 | Multi/ | No | — | — | LINE | L2 | chr11: 73586367-73586684 | − | 140 | 318 | COA4 | Proximal | 0 | Not documented | 72 |
| | Uniquely | | | | | | | | | | | | | | |
| L2b_dup63973 | Multi | No | — | — | LINE | L2 | chr11: 73586790-73586877 | − | 120 | 88 | COA4 | Proximal | 0 | Not documented | 73 |
| MIRb_dup150047 | Multi | No | — | — | SINE | MIR | chr11: 73586901-73587013 | + | 130 | 113 | COA4 | Proximal | 0 | Not documented | 74 |
| AluJb_dup88443 | Multi | No | — | — | SINE | Alu | chr11: 73587029-73587198 | + | 67 | 170 | COA4 | Proximal | 0 | Not documented | 1575 |
| L1MB7_dup15187 | Uniquely | No | — | — | LINE | Other L1 | chr11: 73823457-73824440 | + | 71 | 984 | C2CD3 | Proximal | 0 | Not documented | 76 |
| SVA_B_dup314 | Multi | No | — | — | Other | SVA | chr11: 74038874-74040191 | + | 22 | 1318 | PGM2L1 | Proximal | 1173 | Not documented | 77 |
| AluSp_dup30468 | Multi | No | — | — | SINE | Alu | chr11: 74042309-74042606 | − | 40 | 298 | PGM2L1 | Proximal | 0 | Not documented | 78 |
| AluJr4_dup11376 | Multi | No | — | — | SINE | Alu | chr11: 74045953-74046112 | − | 85 | 160 | PGM2L1 | Proximal | 0 | Not documented | 79 |
| AluSg4_dup4616 | Multi/ | No | — | — | SINE | Alu | chr11: 74046172-74046464 | − | 40 | 293 | PGM2L1 | Proximal | 0 | Not documented | 80 |
| | Uniquely | | | | | | | | | | | | | | |
| ERVL-E-int_dup6302 | Uniquely | No | — | — | LTR | ERVL | chr11: 74625680-74626730 | + | 140 | 1051 | XRRA1 | Proximal | 0 | Not documented | 81 |
| AluSp_dup30502 | Multi/ | No | — | — | SINE | Alu | chr11: 76152396-76152689 | − | 35 | 294 | C11orf30 | Distal | 3279 | Not documented | 82 |
| | Uniquely | | | | | | | | | | | | | | |
| MIR3_dup62178 | Multi | No | — | — | SINE | MIR | chr11: 76508215-76508367 | − | 130 | 153 | TSKU | Proximal | 0 | Not documented | 83 |
| MIRc_dup69915 | Multi/ | No | — | — | SINE | MIR | chr11: 76508498-76508558 | + | 120 | 61 | TSKU | Proximal | 0 | Not documented | 84 |
| | Uniquely | | | | | | | | | | | | | | |
| L1MB5_dup5814 | Multi | No | — | — | LINE | Other L1 | chr11: 767069-767261 | − | 160 | 193 | PDDC1 | Proximal | 0 | Not documented | 85 |
| MIR3_dup62200 | Multi | No | — | — | SINE | MIR | chr11: 76834979-76835082 | + | 96 | 104 | CAPN5 | Proximal | 0 | Not documented | 86 |
| MIRc_dup69940 | Multi | No | — | — | SINE | MIR | chr11: 76836455-76836593 | + | 130 | 139 | CAPN5 | Proximal | 0 | Not documented | 87 |
| MER58A_dup8914 | Uniquely | No | — | — | DNA | DNA | chr11: 77780600-77780793 | − | 80 | 194 | NDUFC2 | Proximal | 0 | Not documented | 88 |
| L1PA14_dup2387 | Uniquely | No | — | — | LINE | L1PA\|B\|x | chr11: 81236135-81240092 | − | 55 | 3958 | FAM181B | Distal | 1202962 | Not documented | 89 |
| LTR10C_dup316 | Uniquely | No | — | — | LTR | ERV1 | chr11: 8160095-8160673 | + | 26 | 579 | RIC3 | Proximal | 0 | Not documented | 90 |
| MIRc_dup66747 | Multi/ | No | — | — | SINE | MIR | chr11: 8161238-8161463 | + | 150 | 226 | RIC3 | Proximal | 0 | Not documented | 91 |
| | Uniquely | | | | | | | | | | | | | | |
| L2a_dup109989 | Multi | No | — | — | LINE | L2 | chr11: 838654-838835 | + | 110 | 182 | POLR2L | Proximal | 0 | Not documented | 92 |
| AluJb_dup84931 | Multi/ | No | — | — | SINE | Alu | chr11: 839635-839943 | − | 85 | 309 | POLR2L | Proximal | 0 | Not documented | 93 |
| | Uniquely | | | | | | | | | | | | | | |
| L2c_dup98028 | Uniquely | No | — | — | LINE | L2 | chr11: 84531301-84531969 | + | 180 | 669 | DLG2 | Proximal | 0 | Not documented | 94 |
| L1MA3_dup6592 | Uniquely | No | — | — | LINE | Other L1 | chr11: 84851489-84857266 | − | 73 | 5778 | DLG2 | Proximal | 0 | Not documented | 95 |
| L2a_dup115428 | Uniquely | No | — | — | LINE | L2 | chr11: 85292746-85293499 | − | 140 | 754 | DLG2 | Proximal | 0 | Not documented | 96 |
| AluY_dup73084 | Multi | No | — | — | SINE | Alu | chr11: 8941175-8941483 | − | 32 | 309 | AKIP1 | Proximal | 0 | Not documented | 97 |
| AluSg_dup24811 | Multi | No | — | — | SINE | Alu | chr11: 9246127-9246423 | + | 35 | 297 | DENND5A | Proximal | 0 | Not documented | 98 |
| L2_dup39717 | Multi | No | — | — | LINE | L2 | chr11: 92914867-92914932 | + | 160 | 66 | SLC36A4 | Proximal | 0 | Not documented | 99 |
| AluSx_dup83724 | Multi | No | — | — | SINE | Alu | chr11: 9303643-9303940 | + | 47 | 298 | TMEM41B | Proximal | 0 | Not documented | 1600 |
| AluY_dup73113 | Multi/ | No | — | — | SINE | Alu | chr11: 9304199-9304508 | + | 22 | 310 | TMEM41B | Proximal | 0 | Not documented | 1 |
| | Uniquely | | | | | | | | | | | | | | |
| FLAM_C_dup13709 | Multi | No | — | — | SINE | Alu | chr11: 93494954-93495090 | + | 66 | 137 | C11orf54 | Proximal | 0 | Not documented | 2 |
| L2c_dup98856 | Multi | No | — | — | LINE | L2 | chr11: 94865486-94865733 | − | 170 | 248 | ENDOD1 | Proximal | 0 | Not documented | 3 |
| MIRc_dup71166 | Uniquely | No | — | — | SINE | MIR | chr11: 95555230-95555381 | + | 140 | 152 | CEP57 | Proximal | 0 | Not documented | 4 |
| Tigger2_dup1971 | Multi | No | — | — | DNA | DNA | chr11: 95556478-95557246 | + | 56 | 769 | CEP57 | Proximal | 0 | Not documented | 5 |
| L3_dup30415 | Multi/ | No | — | — | LINE | CR1 | chr11: 9597763-9597850 | + | 130 | 88 | WEE1 | Proximal | 0 | Not documented | 6 |
| | Uniquely | | | | | | | | | | | | | | |
| MARNA_dup2354 | Multi | No | — | — | DNA | DNA | chr11: 96119014-96119517 | + | 140 | 504 | CCDC82 | Proximal | 0 | Not documented | 7 |
| MIRc_dup71194 | Multi | No | — | — | SINE | MIR | chr11: 96120573-96120764 | − | 130 | 192 | CCDC82 | Proximal | 0 | Not documented | 8 |
| MER1B_dup4028 | Uniquely | No | — | — | DNA | DNA | chr12: 100672878-100673221 | − | 63 | 344 | SCYL2 | Proximal | 0 | Not documented | 9 |
| L1MEc_dup14153 | Uniquely | No | — | — | LINE | Other L1 | chr12: 100673225-100674003 | − | 140 | 779 | SCYL2 | Proximal | 0 | Not documented | 10 |
| MIRc_dup76557 | Multi | No | — | — | SINE | MIR | chr12: 101788613-101788846 | + | 130 | 234 | ARL1 | Proximal | 0 | Not documented | 11 |
| LTR16A_dup5291 | Multi | No | — | — | LTR | ERVL | chr12: 104166310-104166728 | − | 120 | 419 | NT5DC3 | Proximal | 0 | Not documented | 12 |
| MER58A_dup9502 | Multi | No | — | — | DNA | DNA | chr12: 104166965-104167147 | + | 110 | 183 | NT5DC3 | Proximal | 0 | Not documented | 13 |
| AluSq2_dup37099 | Multi | No | — | — | SINE | Alu | chr12: 104168178-104168490 | + | 42 | 313 | NT5DC3 | Proximal | 0 | Not documented | 14 |
| AluSp_dup32994 | Uniquely | No | — | — | SINE | Alu | chr12: 104170052-104170363 | + | 51 | 312 | NT5DC3 | Proximal | 0 | Not documented | 15 |
| AluSx1_dup72421 | Uniquely | No | — | — | SINE | Alu | chr12: 104338334-104338631 | − | 46 | 298 | HSP90B1 | Proximal | 0 | Not documented | 16 |
| L1MA9_dup12867 | Multi | No | — | — | LINE | Other L1 | chr12: 104423654-104424509 | + | 60 | 856 | GLT8D2 | Proximal | 0 | Not documented | 17 |
| AluSc_dup23805 | Multi | No | — | — | SINE | Alu | chr12: 104658750-104659047 | + | 44 | 298 | TXNRD1 | Proximal | 0 | Not documented | 18 |
| AluSz_dup65230 | Uniquely | No | — | — | SINE | Alu | chr12: 104716153-104716457 | − | 58 | 305 | TXNRD1 | Proximal | 0 | Not documented | 19 |
| HAL1_dup20319 | Uniquely | No | — | — | LINE | Other L1 | chr12: 105257240-105259524 | − | 120 | 2285 | SLC41A2 | Proximal | 0 | Not documented | 20 |
| MLT1K_dup12346 | Multi | No | — | — | LTR | ERVL- | chr12: 105666738-105667134 | − | 150 | 397 | C12orf75 | Proximal | 0 | Not documented | 21 |
| | | | | | | MaLR | | | | | | | | | |
| L1ME3_dup6606 | Uniquely | No | — | — | LINE | Other L1 | chr12: 107003473-107003901 | − | 100 | 429 | RFX4 | Proximal | 0 | Not documented | 22 |
| L2b_dup70419 | Multi/ | No | — | — | LINE | L2 | chr12: 107076338-107076565 | − | 140 | 228 | RFX4 | Proximal | 0 | Not documented | 23 |
| | Uniquely | | | | | | | | | | | | | | |
| MER103C_dup6896 | Multi/ | No | — | — | DNA | DNA | chr12: 107076673-107076848 | + | 87 | 176 | RFX4 | Proximal | 0 | Not documented | 24 |
| | Uniquely | | | | | | | | | | | | | | |
| MARNA_dup2556 | Multi/ | No | — | — | DNA | DNA | chr12: 107077010-107077252 | − | 130 | 243 | RFX4 | Proximal | 0 | Not documented | 1625 |
| | Uniquely | | | | | | | | | | | | | | |
| L2b_dup70427 | Uniquely | No | — | — | LINE | L2 | chr12: 107107179-107107377 | − | 150 | 199 | RFX4 | Proximal | 0 | Not documented | 26 |
| AluSq_dup14429 | Multi/ | No | — | — | SINE | Alu | chr12: 107107564-107107869 | + | 50 | 306 | RFX4 | Proximal | 0 | Not documented | 27 |
| | Uniquely | | | | | | | | | | | | | | |
| MIRb_dup165099 | Uniquely | No | — | — | SINE | MIR | chr12: 107110509-107110706 | − | 120 | 198 | RFX4 | Proximal | 0 | Not documented | 28 |
| L2c_dup107623 | Multi | No | — | — | LINE | L2 | chr12: 107131046-107131265 | − | 160 | 220 | RFX4 | Proximal | 0 | Not documented | 29 |
| Tigger16b_dup854 | Uniquely | No | — | — | DNA | DNA | chr12: 107280333-107280612 | − | 110 | 280 | RIC8B | Proximal | 0 | Not documented | 30 |
| L2b_dup70440 | Multi | No | — | — | LINE | L2 | chr12: 107378701-107378929 | + | 120 | 229 | MTERFD3 | Proximal | 0 | Not documented | 31 |
| L2b_dup70441 | Multi/ | No | — | — | LINE | L2 | chr12: 107378979-107379185 | + | 160 | 207 | MTERFD3 | Proximal | 0 | Not documented | 32 |
| | Uniquely | | | | | | | | | | | | | | |
| L1PA11_dup3320 | Uniquely | No | — | — | LINE | L1PA\|B\|x | chr12: 107389924-107390451 | − | 43 | 528 | CRY1 | Proximal | 0 | Not documented | 33 |
| AluSg4_dup4959 | Uniquely | No | — | — | SINE | Alu | chr12: 107397586-107397882 | − | 41 | 297 | CRY1 | Proximal | 0 | Not documented | 34 |
| MIRb_dup165145 | Multi | No | — | — | SINE | MIR | chr12: 107398871-107398908 | − | 130 | 38 | CRY1 | Proximal | 0 | Not documented | 35 |
| L2b_dup70496 | Multi | No | — | — | LINE | L2 | chr12: 108106568-108106645 | − | 140 | 78 | PWP1 | Proximal | 0 | Not documented | 36 |
| Charlie1b_dup2217 | Multi | No | — | — | DNA | DNA | chr12: 10863181-10863614 | + | 84 | 434 | YBX3 | Proximal | 0 | Not documented | 37 |
| MIR_dup128295 | Uniquely | No | — | — | SINE | MIR | chr12: 109898315-109898398 | − | 120 | 84 | KCTD10 | Proximal | 0 | Not documented | 38 |
| MamRep4096_dup891 | Uniquely | No | — | — | DNA | DNA | chr12: 110010544-110010912 | + | 140 | 369 | MMAB | Proximal | 0 | Not documented | 39 |
| AluSq2_dup37211 | Multi | No | — | — | SINE | Alu | chr12: 110289656-110289953 | − | 34 | 298 | GLTP | Proximal | 0 | Not documented | 40 |
| HAL1_dup20347 | Uniquely | No | — | — | LINE | Other L1 | chr12: 110641676-110641756 | + | 95 | 81 | IFT81 | Proximal | 0 | Not documented | 41 |
| L1PA7_dup10665 | Uniquely | No | — | — | LINE | L1PA\|B\|x | chr12: 111066901-111068042 | + | 29 | 1142 | TCTN1 | Proximal | 0 | Not documented | 42 |
| L1MEg2_dup561 | Multi | No | — | — | LINE | Other L1 | chr12: 11126262-11126434 | + | 140 | 173 | TAS2R14 | Proximal | 0 | Not documented | 43 |
| AluSx3_dup18982 | Uniquely | No | — | — | SINE | Alu | chr12: 11148652-11148946 | + | 65 | 295 | TAS2R14 | Proximal | 0 | Not documented | 44 |
| L1MA2_dup5751 | Uniquely | No | — | — | LINE | Other L1 | chr12: 11193318-11195219 | + | 60 | 1902 | TAS2R14 | Proximal | 0 | Not documented | 45 |
| MLT1G_dup2015 | Multi | No | — | — | LTR | ERVL- | chr12: 11199582-11199743 | + | 130 | 162 | TAS2R14 | Proximal | 0 | Not documented | 46 |
| | | | | | | MaLR | | | | | | | | | |
| MLT1J1_dup3724 | Multi | No | — | — | LTR | ERVL- | chr12: 112277524-112277914 | − | 130 | 391 | MAPKAPK5 | Proximal | 1869 | Not documented | 47 |
| | | | | | | MaLR | | | | | | | | | |
| AluJb_dup96790 | Multi | No | — | — | SINE | Alu | chr12: 112278168-112278449 | + | 65 | 282 | MAPKAPK5 | Proximal | 1334 | Not documented | 48 |
| L2a_dup126688 | Multi | No | — | — | LINE | L2 | chr12: 112278878-112279301 | + | 150 | 424 | MAPKAPK5 | Proximal | 482 | Not documented | 49 |
| MIRb_dup166039 | Multi | No | — | — | SINE | MIR | chr12: 112279376-112279535 | + | 140 | 160 | MAPKAPK5 | Proximal | 248 | Not documented | 1650 |
| 7SK_dup566 | Multi | No | — | — | Other | Other | chr12: 112704881-112705119 | + | 13 | 239 | HECTD4 | Proximal | 0 | Not documented | 51 |
| | | | | | | Repeats | | | | | | | | | |
| L2b_dup70870 | Multi | No | — | — | LINE | L2 | chr12: 113596575-113596633 | + | 69 | 59 | CCDC42B | Proximal | 0 | Not documented | 52 |
| MIRb_dup166224 | Uniquely | No | — | — | SINE | MIR | chr12: 113623932-113624117 | + | 120 | 186 | C12orf52 | Proximal | 0 | Not documented | 53 |
| L2c_dup108198 | Multi | No | — | — | LINE | L2 | chr12: 116623807-116624080 | + | 160 | 274 | MED13L | Proximal | 0 | Not documented | 54 |
| MIRc_dup77383 | Multi | No | — | — | SINE | MIR | chr12: 116624328-116624526 | − | 160 | 199 | MED13L | Proximal | 0 | Not documented | 55 |
| MIR_dup129212 | Multi/ | No | — | — | SINE | MIR | chr12: 116625004-116625221 | + | 120 | 218 | MED13L | Proximal | 0 | Not documented | 56 |
| | Uniquely | | | | | | | | | | | | | | |
| L2b_dup71095 | Uniquely | No | — | — | LINE | L2 | chr12: 116973864-116974045 | + | 110 | 182 | MAP1LC3B2 | Distal | 23142 | Not documented | 57 |
| L1M5_dup47404 | Multi | No | — | — | LINE | Other L1 | chr12: 117151132-117151574 | + | 130 | 443 | C12orf49 | Distal | 2020 | Not documented | 58 |
| AluSx1_dup73329 | Multi | No | — | — | SINE | Alu | chr12: 118820472-118820772 | − | 39 | 301 | SUDS3 | Proximal | 0 | Not documented | 59 |
| MIR3_dup67984 | Multi/ | No | — | — | SINE | MIR | chr12: 119617028-119617109 | + | 110 | 82 | HSPB8 | Proximal | 0 | Not documented | 60 |
| | Uniquely | | | | | | | | | | | | | | |
| MamRep564_dup597 | Multi/ | No | — | — | Other | Other | chr12: 119631778-119631842 | − | 130 | 65 | HSPB8 | Proximal | 0 | Not documented | 61 |
| | Uniquely | | | | | Repeats | | | | | | | | | |
| MamRep564_dup598 | Multi/ | No | — | — | Other | Other | chr12: 119631882-119631971 | − | 100 | 90 | HSPB8 | Proximal | 0 | Not documented | 62 |
| | Uniquely | | | | | Repeats | | | | | | | | | |
| AluJr_dup52535 | Multi/ | No | — | — | SINE | Alu | chr12: 120898906-120899192 | − | 70 | 287 | GATC | Proximal | 0 | Not documented | 63 |
| | Uniquely | | | | | | | | | | | | | | |
| MIRb_dup167632 | Multi | No | — | — | SINE | MIR | chr12: 120903343-120903410 | − | 110 | 68 | SRSF9 | Proximal | 0 | Not documented | 64 |
| MIRb_dup167634 | Uniquely | No | — | — | SINE | MIR | chr12: 120928682-120928940 | − | 130 | 259 | DYNLL1 | Proximal | 0 | Not documented | 65 |
| AluJb_dup97955 | Multi/ | No | — | — | SINE | Alu | chr12: 123078172-123078440 | − | 61 | 269 | KNTC1 | Proximal | 0 | Not documented | 66 |
| | Uniquely | | | | | | | | | | | | | | |
| AluSg_dup28202 | Multi | No | — | — | SINE | Alu | chr12: 123254063-123254351 | − | 36 | 289 | DENR | Proximal | 0 | Not documented | 67 |
| L1ME4a_dup32344 | Multi | No | — | — | LINE | Other L1 | chr12: 123254604-123254939 | + | 120 | 336 | DENR | Proximal | 0 | Not documented | 68 |
| AluSx1_dup73843 | Uniquely | No | — | — | SINE | Alu | chr12: 123741773-123742088 | + | 26 | 316 | C12orf65 | Proximal | 0 | Not documented | 69 |
| AluSz6_dup31686 | Multi | No | — | — | SINE | Alu | chr12: 123777769-123777909 | − | 45 | 141 | SBNO1 | Proximal | 0 | Not documented | 70 |
| AluSz_dup66682 | Uniquely | No | — | — | SINE | Alu | chr12: 123955608-123955918 | − | 54 | 311 | SNRNP35 | Proximal | 0 | Not documented | 71 |
| L4_dup13074 | Multi | No | — | — | LINE | RTE | chr12: 124102402-124102457 | − | 110 | 56 | DDX55 | Proximal | 0 | Not documented | 72 |
| AluSq2_dup37685 | Uniquely | No | — | — | SINE | Alu | chr12: 124180394-124180690 | − | 49 | 297 | TCTN2 | Proximal | 0 | Not documented | 73 |
| L1MC4a_dup20035 | Uniquely | No | — | — | LINE | Other L1 | chr12: 124416809-124417767 | − | 140 | 959 | DNAH10OS | Proximal | 0 | Not documented | 74 |
| MIR3_dup68136 | Multi/ | Nc | — | — | SINE | MIR | chr12: 124499406-124499534 | + | 120 | 129 | ZNF664 | Proximal | 0 | Not documented | 1675 |
| | Uniquely | | | | | | | | | | | | | | |
| MIR3_dup68137 | Multi/ | No | — | — | SINE | MIR | chr12: 124499624-124499809 | + | 120 | 186 | ZNF664 | Proximal | 0 | Not documented | 76 |
| | Uniquely | | | | | | | | | | | | | | |
| MLT1B_dup13443 | Multi | No | — | — | LTR | ERVL- | chr12: 127908982-127909380 | + | 95 | 399 | TMEM132C | Distal | 842569 | Not documented | 77 |
| | | | | | | MaLR | | | | | | | | | |
| AluSx3_dup20231 | Multi | No | — | — | SINE | Alu | chr12: 131275791-131276086 | + | 37 | 296 | STX2 | Proximal | 0 | Not documented | 78 |
| AluSq2_dup35616 | Multi | No | — | — | SINE | Alu | chr12: 13234884-13235197 | − | 35 | 314 | KIAA1467 | Proximal | 0 | Not documented | 79 |
| AluSg_dup28350 | Multi | No | — | — | SINE | Alu | chr12: 133298830-133299133 | − | 35 | 304 | PGAM5 | Proximal | 0 | Not documented | 80 |
| L1MEc_dup14329 | Uniquely | No | — | — | LINE | Other L1 | chr12: 133500813-133501403 | − | 130 | 591 | ZNF605 | Proximal | 0 | Not documented | 81 |
| AluSx1_dup69539 | Multi/ | No | — | — | SINE | Alu | chr12: 13350428-13350738 | + | 54 | 311 | EMP1 | Proximal | 0 | Not documented | 82 |
| | Uniquely | | | | | | | | | | | | | | |
| Plat_L3_dup2748 | Uniquely | No | — | — | LINE | CR1 | chr12: 13352500-13352640 | − | 160 | 141 | EMP1 | Proximal | 0 | Not documented | 83 |
| MIR3_dup65091 | Multi/ | No | — | — | SINE | MIR | chr12: 13354015-13354071 | + | 120 | 57 | EMP1 | Proximal | 0 | Not documented | 84 |
| | Uniquely | | | | | | | | | | | | | | |
| AluJo_dup45726 | Multi/ | No | — | — | SINE | Alu | chr12: 13354083-13354364 | + | 73 | 282 | EMP1 | Proximal | 0 | Not documented | 85 |
| | Uniquely | | | | | | | | | | | | | | |
| MIRb_dup158425 | Multi/ | No | — | — | SINE | MIR | chr12: 13362499-13362646 | − | 150 | 148 | EMP1 | Proximal | 0 | Not documented | 86 |
| | Uniquely | | | | | | | | | | | | | | |
| AmnSINE1_dup795 | Multi | No | — | — | SINE | Deu | chr12: 13368777-13368814 | + | 60 | 38 | EMP1 | Proximal | 0 | Not documented | 87 |
| MER135_dup648 | Multi/ | No | — | — | DNA | DNA | chr12: 13369826-13369896 | + | 110 | 71 | EMP1 | Proximal | 119 | Not documented | 88 |
| | Uniquely | | | | | | | | | | | | | | |
| THE1D-int_dup1482 | Uniquely | No | — | — | LTR | ERVL- | chr12: 13372244-13372493 | − | 51 | 250 | EMP1 | Distal | 2537 | Not documented | 89 |
| | | | | | | MaLR | | | | | | | | | |
| MER115_dup1883 | Uniquely | No | — | — | DNA | DNA | chr12: 13374756-13375390 | − | 140 | 635 | EMP1 | Distal | 5049 | Not documented | 90 |
| MER131_dup337 | Multi | No | — | — | SINE | SINE | chr12: 16703494-16703570 | − | 96 | 77 | LMO3 | Proximal | 0 | Not documented | 91 |
| L2a_dup119993 | Multi | No | — | — | LINE | L2 | chr12: 19349952-19350213 | + | 160 | 262 | PLEKHA5 | Proximal | 0 | Not documented | 92 |
| L1PA8_dup6066 | Multi | No | — | — | LINE | L1PA\|B\|x | chr12: 20229577-20230192 | + | 37 | 616 | PDE3A | Distal | 291988 | Not documented | 93 |
| MER 103C_dup6613 | Multi | No | — | — | DNA | DNA | chr12: 20605457-20605532 | + | 100 | 76 | PDE3A | Proximal | 0 | Not documented | 94 |
| HAL1_dup19595 | Multi | No | — | — | LINE | Other L1 | chr12: 20851301-20851441 | − | 110 | 141 | SLCO1C1 | Proximal | 0 | Not documented | 95 |
| MER4-int_dup2181 | Uniquely | No | — | — | LTR | ERV1 | chr12: 22456823-22458313 | + | 52 | 1491 | ST8SIA1 | Proximal | 0 | Not documented | 96 |
| MER44B_dup1546 | Uniquely | No | — | — | DNA | DNA | chr12: 22478526-22479065 | − | 83 | 540 | ST8SIA1 | Proximal | 0 | Not documented | 97 |
| MSTB_dup6150 | Multi | No | — | — | LTR | ERVL- | chr12: 26108009-26108382 | − | 85 | 374 | RASSF8 | Distal | 3581 | Not documented | 98 |
| | | | | | | MaLR | | | | | | | | | |
| L2c_dup103135 | Uniquely | No | — | — | LINE | L2 | chr12: 26123407-26123563 | + | 150 | 157 | RASSF8 | Proximal | 0 | Not documented | 99 |
| L1MB8_dup11706 | Uniquely | No | — | — | LINE | Other L1 | chr12: 26174357-26175667 | + | 89 | 1311 | RASSF8 | Proximal | 0 | Not documented | 1700 |
| L2b_dup67818 | Uniquely | No | — | — | LINE | L2 | chr12: 27952505-27952609 | − | 110 | 105 | KLHL42 | Proximal | 0 | Not documented | 1 |
| AluSz6_dup29891 | Multi | No | — | — | SINE | Alu | chr12: 27953569-27953666 | + | 37 | 98 | KLHL42 | Proximal | 0 | Not documented | 2 |
| MER117_dup3184 | Uniquely | No | — | — | DNA | DNA | chr12: 27954492-27954668 | − | 140 | 177 | KLHL42 | Proximal | 0 | Not documented | 3 |
| AluSx1_dup69130 | Multi | No | — | — | SINE | Alu | chr12: 2997695-2997965 | − | 60 | 271 | RHNO1 | Proximal | 0 | Not documented | 4 |
| AluSp_dup31811 | Uniquely | No | — | — | SINE | Alu | chr12: 32264936-32265239 | − | 46 | 304 | BICD1 | Proximal | 0 | Not documented | 5 |
| L2a_dup118837 | Multi | No | — | — | LINE | L2 | chr12: 385654-385707 | − | 110 | 54 | KDM5A | Distal | 3589 | Not documented | 6 |
| AluSz_dup63200 | Multi | No | — | — | SINE | | chr12: 42835488-42835621 | − | 44 | 134 | PPHLN1 | Proximal | 0 | Not documented | 7 |
| MamRep137_dup1600 | Uniquely | No | — | — | DNA | DNA | chr12: 4406004-4406341 | − | 120 | 338 | CCND2 | Proximal | 0 | Not documented | 8 |
| L2_dup41522 | Multi | No | — | — | LINE | L2 | chr12: 45429782-45429945 | − | 140 | 164 | DBX2 | Proximal | 0 | Not documented | 9 |
| AluJb_dup90884 | Uniquely | No | — | — | SINE | Alu | chr12: 466165-466441 | + | 70 | 277 | KDM5A | Proximal | 0 | Not documented | 10 |
| L1ME4a_dup31047 | Multi | No | — | — | LINE | Other L1 | chr12: 46636903-46637064 | − | 110 | 162 | SLC38A1 | Proximal | 0 | Not documented | 11 |
| L2a_dup121598 | Multi | No | — | — | LINE | L2 | chr12: 46637065-46637114 | + | 64 | 50 | SLC38A1 | Proximal | 0 | Not documented | 12 |
| MIR3_dup65802 | Multi | No | — | — | SINE | MIR | chr12: 48176023-48176161 | − | 130 | 139 | SLC48A1 | Proximal | 0 | Not documented | 13 |
| L2a_dup121723 | Multi/ | No | — | — | LINE | L2 | chr12: 48362213-48362267 | + | 91 | 55 | TMEM106C | Proximal | 0 | Not documented | 14 |
| | Uniquely | | | | | | | | | | | | | | |
| AluSq2_dup36066 | Multi | No | — | — | SINE | Alu | chr12: 48542442-48542746 | − | 40 | 305 | ASB8 | Proximal | 0 | Not documented | 15 |
| AluSq2_dup36106 | Uniquely | No | — | — | SINE | Alu | chr12: 49406150-49406456 | − | 55 | 307 | PRKAG1 | Proximal | 0 | Not documented | 16 |
| AluJo_dup46510 | Multi | No | — | — | SINE | Alu | chr12: 51325532-51325647 | − | 78 | 116 | METTL7A | Proximal | 0 | Not documented | 17 |
| AluJb_dup93598 | Uniquely | No | — | — | SINE | Alu | chr12: 53396894-53397190 | + | 69 | 297 | EIF4B | Distal | 2753 | Not documented | 18 |
| AluSz_dup63665 | Uniquely | No | — | — | SINE | Alu | chr12: 53551923-53552219 | − | 47 | 297 | CSAD | Proximal | 0 | Not documented | 19 |
| L2a_dup122096 | Multi | No | — | — | LINE | L2 | chr12: 54526477-54526632 | − | 140 | 156 | SMUG1 | Distal | 31898 | Not documented | 20 |
| MIR_dup124072 | Multi/ | No | — | — | SINE | MIR | chr12: 54626190-54626307 | − | 42 | 118 | CBX5 | Proximal | 0 | Not documented | 21 |
| | Uniquely | | | | | | | | | | | | | | |
| AluJo_dup46649 | Multi/ | No | — | — | SINE | Alu | chr12: 54627433-54627675 | + | 60 | 243 | CBX5 | Proximal | 0 | Not documented | 22 |
| | Uniquely | | | | | | | | | | | | | | |
| AluSg7_dup5456 | Multi | No | — | — | SINE | Alu | chr12: 54627687-54627990 | + | 36 | 304 | CBX5 | Proximal | 0 | Not documented | 23 |
| AluJr_dup50528 | Multi/ | No | — | — | SINE | Alu | chr12: 54634180-54634310 | − | 52 | 131 | CBX5 | Proximal | 0 | Not documented | 24 |
| | Uniquely | | | | | | | | | | | | | | |
| MIRc_dup75061 | Uniquely | No | — | — | SINE | MIR | chr12: 56114371-56114513 | − | 120 | 143 | RDH5 | Proximal | 0 | Not documented | 1725 |
| HAL1-3A_ME_{—} | Multi | No | — | — | LINE | Other L1 | chr12: 56373947-56374079 | − | 120 | 133 | RAB5B | Proximal | 0 | Not documented | 26 |
| dup1369 | | | | | | | | | | | | | | | |
| MER50-int_dup396 | Multi | No | — | — | LTR | ERV1 | chr12: 56500341-56500448 | − | 110 | 108 | RP11-603J24.9 | Proximal | 0 | Not documented | 27 |
| AluSp_dup32340 | Multi | No | — | — | SINE | Alu | chr12: 56623237-56623548 | + | 41 | 312 | NABP2 | Proximal | 0 | Not documented | 28 |
| L1MB3_dup11739 | Multi | No | — | — | LINE | Other L1 | chr12: 56679965-56680145 | + | 70 | 181 | CS | Proximal | 0 | Not documented | 29 |
| AluJb_dup93880 | Multi | No | — | — | SINE | Alu | chr12: 56706004-56706304 | − | 68 | 301 | CNPY2 | Proximal | 0 | Not documented | 30 |
| AluSp_dup32348 | Uniquely | No | — | — | SINE | Alu | chr12: 56706347-56706653 | − | 34 | 307 | CNPY2 | Proximal | 0 | Not documented | 31 |
| AluSp_dup32352 | Multi | No | — | — | SINE | Alu | chr12: 56735800-56736099 | + | 41 | 300 | STAT2 | Proximal | 0 | Not documented | 32 |
| AluJo_dup46715 | Multi | No | — | — | SINE | Alu | chr12: 56736104-56736385 | + | 61 | 282 | STAT2 | Proximal | 0 | Not documented | 33 |
| AluSx_dup91977 | Multi/ | No | — | — | SINE | Alu | chr12: 58141528-58141824 | − | 43 | 297 | CDK4 | Proximal | 0 | Not documented | 34 |
| | Uniquely | | | | | | | | | | | | | | |
| AluSx_dup91978 | Multi | No | — | — | SINE | Alu | chr12: 58142478-58142788 | − | 42 | 311 | CDK4 | Proximal | 0 | Not documented | 35 |
| AluJr_dup50670 | Uniquely | No | — | — | SINE | Alu | chr12: 58144102-58144361 | + | 79 | 260 | CDK4 | Proximal | 0 | Not documented | 36 |
| AluSg_dup27261 | Multi | No | — | — | SINE | Alu | chr12: 58151156-58151459 | + | 28 | 304 | MARCH9 | Proximal | 0 | Not documented | 37 |
| AluJb_dup94094 | Multi/ | No | — | — | SINE | Alu | chr12: 58174833-58175135 | − | 57 | 303 | METTL21B | Proximal | 0 | Not documented | 38 |
| | Uniquely | | | | | | | | | | | | | | |
| L2c_dup104626 | Multi/ | No | — | — | LINE | L2 | chr12: 58176029-58176167 | + | 110 | 139 | METTL21B | Proximal | 0 | Not documented | 39 |
| | Uniquely | | | | | | | | | | | | | | |
| MIRc_dup75116 | Multi | No | — | — | SINE | MIR | chr12: 58176242-58176341 | + | 120 | 100 | RP11-571M6.15 | Proximal | 0 | Not documented | 40 |
| L2_dup41795 | Uniquely | No | — | — | LINE | L2 | chr12: 58196306-58196695 | + | 170 | 390 | AVIL | Proximal | 0 | Not documented | 41 |
| L1ME1_dup22971 | Uniquely | No | — | — | LINE | Other L1 | chr12: 58197771-58198313 | − | 88 | 543 | AVIL | Proximal | 0 | Not documented | 42 |
| MIRb_dup161150 | Uniquely | No | — | — | SINE | MIR | chr12: 58200484-58200707 | − | 130 | 224 | AVIL | Proximal | 0 | Not documented | 43 |
| L2c_dup104628 | Multi/ | No | — | — | LINE | L2 | chr12: 58210829-58210931 | − | 130 | 103 | AVIL | Proximal | 0 | Not documented | 44 |
| | Uniquely | | | | | | | | | | | | | | |
| AluJb_dup94096 | Multi | No | — | — | SINE | Alu | chr12: 58211799-58212112 | − | 65 | 314 | AVIL | Proximal | 0 | Not documented | 45 |
| AluSg7_dup5483 | Multi/ | No | — | — | SINE | Alu | chr12: 58241206-58241504 | + | 14 | 299 | CTDSP2 | Proximal | 685 | Not documented | 46 |
| | Uniquely | | | | | | | | | | | | | | |
| MLT1K_dup11951 | Multi/ | No | — | — | LTR | ERVL- | chr12: 58325258-58325488 | − | 120 | 231 | XRCC6BP1 | Distal | 9837 | Not documented | 47 |
| | Uniquely | | | | | MaLR | | | | | | | | | |
| L2_dup41797 | Multi/ | No | — | — | LINE | L2 | chr12: 58329239-58329350 | + | 130 | 112 | XRCC6BP1 | Distal | 5975 | Not documented | 48 |
| | Uniquely | | | | | | | | | | | | | | |
| L1PB1_dup10387 | Uniquely | No | — | — | LINE | L1PA\|B\|x | chr12: 58351230-58357709 | + | 42 | 6480 | XRCC6BP1 | Proximal | 179 | EVE annotated | 49 |
| HERVK-int_dup183 | Multi | No | — | — | LTR | ERVK | chr12: 58722210-58729730 | − | 3.6 | 7521 | RP11-362K2.2 | Distal | 208178 | EVE annotated | 1750 |
| MLT1F1_dup2264 | Uniquely | No | — | — | LTR | ERVL- | chr12: 58873116-58873590 | − | 110 | 475 | RP11-362K2.2 | Distal | 64318 | Not documented | 51 |
| | | | | | | MaLR | | | | | | | | | |
| L1MCa_dup5535 | Uniquely | No | — | — | LINE | Other L1 | chr12: 60759417-60761017 | + | 93 | 1601 | SLC16A7 | Distal | 583023 | Not documented | 52 |
| L1MCa_dup5536 | Uniquely | No | — | — | LINE | Other L1 | chr12: 60764521-60766482 | + | 83 | 1962 | SLC16A7 | Distal | 588127 | Not documented | 53 |
| L1PA10_dup5376 | Uniquely | No | — | — | LINE | L1PA\|B\|x | chr12: 60766483-60768294 | + | 84 | 1812 | SLC16A7 | Distal | 590089 | Not documented | 54 |
| L1PA10_dup5377 | Uniquely | No | — | — | LINE | L1PA\|B\|x | chr12: 60768560-60770725 | + | 56 | 2166 | SLC16A7 | Distal | 592166 | Not documented | 55 |
| LTR26_dup449 | Uniquely | No | — | — | LTR | ERV1 | chr12: 60809173-60809756 | + | 73 | 584 | SLC16A7 | Distal | 632779 | Not documented | 56 |
| L1HS_dup1230 | Multi | No | — | — | LINE | L1PA\|B\|x | chr12: 62295058-62295218 | − | 2.7 | 161 | FAM19A2 | Proximal | 0 | Not documented | 57 |
| MLT1D-int_dup503 | Multi/ | No | — | — | LTR | ERVL- | chr12: 64175992-64176963 | − | 110 | 972 | TMEM5 | Proximal | 0 | Not documented | 58 |
| | Uniquely | | | | | MaLR | | | | | | | | | |
| Tigger3b_dup4931 | Multi/ | No | — | — | DNA | DNA | chr12: 64202998-64203287 | + | 64 | 290 | TMEM5 | Proximal | 0 | Not documented | 59 |
| | Uniquely | | | | | | | | | | | | | | |
| FLAM_A_dup11157 | Multi/ | No | — | — | SINE | Alu | chr12: 64537442-64537574 | + | 75 | 133 | SRGAP1 | Proximal | 0 | Not documented | 60 |
| | Uniquely | | | | | | | | | | | | | | |
| Plat_L3_dup2824 | Multi | No | — | — | LINE | CR1 | chr12: 64537952-64538013 | − | 170 | 62 | SRGAP1 | Proximal | 0 | Not documented | 61 |
| Plat_L3_dup2825 | Multi | No | — | — | LINE | CR1 | chr12: 64538035-64538329 | − | 170 | 295 | SRGAP1 | Proximal | 0 | Not documented | 62 |
| AluSx_dup92124 | Multi/ | No | — | — | SINE | Alu | chr12: 64538617-64538910 | − | 39 | 294 | SRGAP1 | Proximal | 0 | Not documented | 63 |
| | Uniquely | | | | | | | | | | | | | | |
| AluSz_dup64099 | Multi | No | — | — | SINE | Alu | chr12: 64842697-64842992 | + | 48 | 296 | XPOT | Proximal | 0 | Not documented | 64 |
| AluJr4_dup12146 | Multi | No | — | — | SINE | Alu | chr12: 64843934-64844224 | − | 75 | 291 | XPOT | Proximal | 0 | Not documented | 65 |
| L2a_dup122684 | Uniquely | No | — | — | LINE | L2 | chr12: 64862307-64862608 | − | 140 | 302 | TBK1 | Proximal | 0 | Not documented | 66 |
| AluSz6_dup30520 | Multi | No | — | — | SINE | Alu | chr12: 64894534-64894837 | − | 42 | 304 | TBK1 | Proximal | 0 | Not documented | 67 |
| AluSx3_dup19537 | Multi | No | — | — | SINE | Alu | chr12: 65109018-65109316 | + | 44 | 299 | GNS | Proximal | 0 | Not documented | 68 |
| MLT1D_dup14096 | Multi | No | — | — | LTR | ERVL- | chr12: 6548497-6548974 | − | 93 | 478 | CD27 | Distal | 5060 | Not documented | 69 |
| | | | | | | MaLR | | | | | | | | | |
| AluJb_dup91226 | Multi/ | No | — | — | SINE | Alu | chr12: 6555977-6556282 | + | 64 | 30€ | CD27 | Proximal | 0 | Not documented | 70 |
| | Uniquely | | | | | | | | | | | | | | |
| AluSc8_dup14064 | Multi | No | — | — | SINE | Alu | chr12: 6557665-6557951 | + | 33 | 287 | CD27 | Proximal | 0 | Not documented | 71 |
| MIRb_dup158063 | Multi | No | — | — | SINE | MIR | chr12: 6602717-6602868 | − | 130 | 152 | MRPL51 | Proximal | 0 | Not documented | 72 |
| L2c_dup102152 | Multi | No | — | — | LINE | L2 | chr12: 6651191-6651264 | + | 100 | 74 | IFFO1 | Proximal | 0 | Not documented | 73 |
| AluSx3_dup19553 | Multi/ | No | — | — | SINE | Alu | chr12: 66516915-66517204 | − | 50 | 290 | LLPH | Proximal | 0 | Not documented | 74 |
| | Uniquely | | | | | | | | | | | | | | |
| MER57B2_dup796 | Uniquely | No | — | — | LTR | ERV1 | chr12: 66562244-66562617 | + | 67 | 374 | TMBIM4 | Proximal | 0 | Not documented | 1775 |
| AluSp_dup31380 | Multi | No | — | — | SINE | Alu | chr12: 6842600-6842912 | − | 46 | 313 | COPS7A | Proximal | 1560 | Not documented | 76 |
| AluSz_dup64256 | Multi | No | — | — | SINE | Alu | chr12: 69234616-69234915 | − | 60 | 300 | MDM2 | Proximal | 0 | Not documented | 77 |
| AluJo_dup47023 | Multi | No | — | — | SINE | Alu | chr12: 69235059-69235179 | − | 45 | 121 | MDM2 | Proximal | 0 | Not documented | 78 |
| L4_dup12746 | Multi/ | No | — | — | LINE | RTE | chr12: 69653917-69653978 | + | 150 | 62 | CPSF6 | Proximal | 0 | Not documented | 79 |
| | Uniquely | | | | | | | | | | | | | | |
| AluSp_dup32570 | Multi/ | No | — | — | SINE | Alu | chr12: 69659379-69659685 | − | 46 | 307 | CPSF6 | Proximal | 0 | Not documented | 80 |
| | Uniquely | | | | | | | | | | | | | | |
| FRAM_dup5482 | Multi | No | — | — | SINE | Alu | chr12: 6991340-6991482 | + | 92 | 143 | SPSB2 | Proximal | 0 | Not documented | 81 |
| L1PA6_dup4723 | Uniquely | No | — | — | LINE | L1PA\|B\|x | chr12: 70576144-70577580 | − | 30 | 1437 | CNOT2 | Distal | 59195 | Not documented | 82 |
| L1MA9_dup12645 | Uniquely | No | — | — | LINE | Other L1 | chr12: 70602424-70604608 | − | 72 | 2185 | CNOT2 | Distal | 32167 | Not documented | 83 |
| AluY_dup80634 | Multi/ | No | — | — | SINE | Alu | chr12: 70655083-70655395 | − | 36 | 313 | CNOT2 | Proximal | 0 | Not documented | 84 |
| | Uniquely | | | | | | | | | | | | | | |
| L1ME1_dup23110 | Multi | No | — | — | LINE | Other L1 | chr12: 70655396-70656047 | − | 85 | 652 | CNOT2 | Proximal | 0 | Not documented | 85 |
| L1ME1_dup23111 | Multi | No | — | — | LINE | Other L1 | chr12: 70656367-70656443 | − | 120 | 77 | CNOT2 | Proximal | 0 | Not documented | 86 |
| AluSx1_dup71499 | Multi | No | — | — | SINE | Alu | chr12: 70656444-70656752 | − | 60 | 309 | CNOT2 | Proximal | 0 | Not documented | 87 |
| L1ME1_dup23112 | Multi | No | — | — | LINE | Other L1 | chr12: 70656753-70658721 | − | 110 | 1969 | CNOT2 | Proximal | 0 | Not documented | 88 |
| AluSg_dup27389 | Multi | No | — | — | SINE | Alu | chr12: 70674865-70675160 | − | 31 | 296 | CNOT2 | Proximal | 0 | Not documented | 89 |
| L2a_dup123233 | Uniquely | No | — | — | LINE | L2 | chr12: 70734966-70735467 | + | 130 | 502 | CNOT2 | Proximal | 0 | Not documented | 90 |
| L3_dup34074 | Multi | No | — | — | LINE | CR1 | chr12: 70748179-70748289 | − | 110 | 111 | CNOT2 | Proximal | 0 | Not documented | 91 |
| AluJb_dup91332 | Multi | No | — | — | SINE | Alu | chr12: 7134869-7135223 | − | 63 | 355 | C15 | Proximal | 0 | Not documented | 92 |
| L1MB7_dup15684 | Multi | No | — | — | LINE | Other L1 | chr12: 7250942-7251509 | + | 78 | 568 | C1RL | Proximal | 0 | Not documented | 93 |
| AluSq2_dup35428 | Multi | No | — | — | SINE | Alu | chr12: 7251510-7251850 | + | 41 | 341 | C1RL | Proximal | 0 | Not documented | 94 |
| MIRc_dup73591 | Multi | No | — | — | SINE | MIR | chr12: 7411392-7411619 | + | 140 | 228 | PEX5 | Distal | 40223 | Not documented | 95 |
| MER4B-int_dup1079 | Multi | No | — | — | LTR | ERV1 | chr12: 7445139-7448128 | + | 54 | 2990 | ACSM4 | Distal | 8753 | Not documented | 96 |
| L2c_dup105959 | Uniquely | No | — | — | LINE | L2 | chr12: 81541147-81541515 | − | 160 | 369 | ACSS3 | Proximal | 0 | Not documented | 97 |
| L1MC1_dup10016 | Multi | No | — | — | LINE | Other L1 | chr12: 81644889-81645325 | + | 91 | 437 | ACSS3 | Proximal | 0 | Not documented | 98 |
| MER102a_dup1873 | Multi | No | — | — | DNA | DNA | chr12: 81649887-81650088 | − | 140 | 202 | ACSS3 | Proximal | 0 | Not documented | 99 |
| L1P4a_dup461 | Uniquely | No | — | — | LINE | L1PA\|B\|x | chr12: 82000001-82005952 | − | 75 | 5952 | PPFIA2 | Proximal | 0 | Not documented | 1800 |
| LTR12C_dup2128 | Uniquely | No | — | — | LTR | ERV1 | chr12: 83122367-83123937 | + | 35 | 1571 | TMTC2 | Proximal | 0 | Not documented | 1 |
| AluSp_dup32751 | Uniquely | No | — | — | SINE | Alu | chr12: 87551832-87552137 | + | 50 | 306 | MGAT4C | Distal | 319152 | Not documented | 2 |
| MER5B_dup18168 | Multi | No | — | — | DNA | DNA | chr12: 88591566-88591729 | + | 110 | 164 | TMTC3 | Proximal | 0 | Not documented | 3 |
| L1MA5A_dup2594 | Uniquely | No | — | — | LINE | Other L1 | chr12: 92391143-92393003 | + | 73 | 1861 | C12orf79 | Proximal | 0 | Not documented | 4 |
| AluSg_dup27582 | Multi | No | — | — | SINE | Alu | chr12: 93794397-93794682 | − | 41 | 286 | NUDT4 | Proximal | 0 | Not documented | 5 |
| Tigger5_dup1982 | Multi | No | — | — | DNA | DNA | chr12: 93895235-93895274 | + | 80 | 40 | MRPL42 | Proximal | 0 | Not documented | 6 |
| Tigger5_dup1983 | Multi | No | — | — | DNA | DNA | chr12: 93895265-93895466 | + | 74 | 202 | MRPL42 | Proximal | 0 | Not documented | 7 |
| MIRb_dup163705 | Multi | No | — | — | SINE | MIR | chr12: 93895692-93895851 | − | 110 | 160 | MRPL42 | Proximal | 0 | Not documented | 8 |
| L2_dup42601 | Uniquely | No | — | — | LINE | L2 | chr12: 94731868-94732459 | − | 190 | 592 | CCDC41 | Proximal | 0 | Not documented | 9 |
| HSMAR1_dup559 | Uniquely | No | — | — | DNA | DNA | chr12: 94742022-94742781 | + | 50 | 760 | CCDC41 | Proximal | 0 | Not documented | 10 |
| L1MC4a_dup19708 | Uniquely | No | — | — | LINE | Other L1 | chr12: 94759572-94761543 | + | 110 | 1972 | CCDC41 | Proximal | 0 | Not documented | 11 |
| L1MDa_dup5295 | Uniquely | No | — | — | LINE | Other L1 | chr12: 94777340-94779278 | − | 93 | 1939 | CCDC41 | Proximal | 0 | Not documented | 12 |
| L4_dup12916 | Multi | No | — | — | LINE | RTE | chr12: 96674001-96674055 | − | 71 | 55 | CDK17 | Proximal | 0 | Not documented | 13 |
| L2b_dup69932 | Uniquely | No | — | — | LINE | L2 | chr12: 97873231-97873856 | + | 160 | 626 | NEDD1 | Distal | 526103 | Not documented | 14 |
| MLT1B_dup14071 | Multi/ | No | — | — | LTR | ERVL- | chr13: 110683402-110683770 | − | 100 | 369 | COL4A1 | Distal | 117549 | Not documented | 15 |
| | Uniquely | | | | | MaLR | | | | | | | | | |
| L2c_dup112859 | Multi | No | — | — | LINE | L2 | chr13: 110684537-110684625 | + | 150 | 89 | COL4A1 | Distal | 116694 | Not documented | 16 |
| MLT1A_dup7150 | Multi | No | — | — | LTR | ERVL- | chr13: 110687676-110687946 | + | 120 | 271 | COL4A1 | Distal | 113373 | Not documented | 17 |
| | | | | | | MaLR | | | | | | | | | |
| MER90a_dup936 | Multi/ | No | — | — | LTR | ERV1 | chr13: 110689097-110689207 | − | 110 | 111 | COL4A1 | Distal | 112112 | Not documented | 18 |
| | Uniquely | | | | | | | | | | | | | | |
| AluJr_dup54824 | Multi/ | No | — | — | SINE | Alu | chr13: 110708461-110708755 | + | 69 | 295 | COL4A1 | Distal | 92564 | Not documented | 19 |
| | Uniquely | | | | | | | | | | | | | | |
| LTR58_dup72 | Multi/ | No | — | — | LTR | ERV1 | chr13: 110709015-110709666 | + | 97 | 652 | COL4A1 | Distal | 91653 | Not documented | 20 |
| | Uniquely | | | | | | | | | | | | | | |
| MER47A_dup2353 | Multi | No | — | — | DNA | DNA | chr13: 113831750-113831983 | + | 110 | 234 | PCID2 | Proximal | 0 | Not documented | 21 |
| AluSg_dup29387 | Multi/ | No | — | — | SINE | Alu | chr13: 115049557-115049827 | + | 50 | 271 | UPF3A | Proximal | 0 | Not documented | 22 |
| | Uniquely | | | | | | | | | | | | | | |
| MER30_dup3106 | Uniquely | No | — | — | DNA | DNA | chr13: 21142053-21142265 | − | 39 | 213 | IFT88 | Proximal | 0 | Not documented | 23 |
| L1MA4A_dup4945 | Uniquely | No | — | — | LINE | Other L1 | chr13: 21324223-21328766 | − | 68 | 4544 | N6AMT2 | Proximal | 0 | Not documented | 24 |
| LTR6A_dup176 | Multi | No | — | — | LTR | ERV1 | chr13: 21535457-21535486 | − | 15 | 30 | LATS2 | Distal | 11686 | Not documented | 1825 |
| MIR_dup130911 | Multi | No | — | — | SINE | MIR | chr13: 21722348-21722485 | + | 110 | 138 | SAP18 | Proximal | 0 | Not documented | 26 |
| AluSz_dup67206 | Multi | No | — | — | SINE | Alu | chr13: 21751827-21752130 | + | 60 | 304 | MRP63 | Proximal | 0 | Not documented | 27 |
| L1MC1_dup10254 | Uniquely | No | — | — | LINE | Other L1 | chr13: 24175197-24180260 | + | 65 | 5064 | TNFRSF19 | Proximal | 0 | Not documented | 28 |
| L1MA8_dup8429 | Uniquely | No | — | — | LINE | Other L1 | chr13: 24783639-24785676 | − | 73 | 2038 | SPATA13 | Proximal | 0 | Not documented | 29 |
| L1ME4a_dup32610 | Uniquely | No | — | — | LINE | Other L1 | chr13: 27252425-27252685 | − | 110 | 261 | WASF3 | Proximal | 0 | Not documented | 30 |
| L1MC5_dup15083 | Multi | No | — | — | LINE | Other L1 | chr13: 28019028-28019335 | + | 130 | 308 | MTIF3 | Proximal | 0 | Not documented | 31 |
| MER21-int_dup772 | Uniquely | No | — | — | LTR | ERVL | chr13: 31872487-31874496 | − | 85 | 2010 | B3GALTL | Proximal | 0 | Not documented | 32 |
| AluSc5_dup4853 | Multi | No | — | — | SINE | Alu | chr13: 38936643-38936730 | − | 31 | 88 | UFM1 | Proximal | 0 | Not documented | 33 |
| L1PA15_dup6807 | Uniquely | No | — | — | LINE | L1PA\|B\|x | chr13: 40034963-40035603 | − | 48 | 641 | LHFP | Proximal | 0 | Not documented | 34 |
| MER5B_dup18875 | Multi/ | No | — | — | DNA | DNA | chr13: 45945403-45945517 | − | 140 | 115 | SLC25A30 | Distal | 21935 | Not documented | 35 |
| | Uniquely | | | | | | | | | | | | | | |
| Tigger2_dup2156 | Uniquely | No | — | — | DNA | DNA | chr13: 50626005-50627469 | + | 58 | 1465 | DLEU1 | Distal | 28839 | Not documented | 36 |
| MIRb_dup170640 | Uniquely | No | — | — | SINE | MIR | chr13: 50656622-50656844 | − | 110 | 223 | DLEU1 | Proximal | 0 | Not documented | 37 |
| AluSx4_dup4047 | Multi | No | — | — | SINE | Alu | chr13: 67780292-67780588 | − | 35 | 297 | PCDH9 | Proximal | 0 | Not documented | 38 |
| AluY_dup85247 | Multi | No | — | — | SINE | Alu | chr13: 67787067-67787368 | + | 21 | 302 | PCDH9 | Proximal | 0 | Not documented | 39 |
| L1PBa1_dup327 | Uniquely | No | — | — | LINE | L1PA\|B\|x | chr13: 80835279-80837619 | − | 60 | 2341 | SPRY2 | Distal | 72493 | Not documented | 40 |
| FLAM_A_dup11969 | Multi | No | — | — | SINE | Alu | chr13: 88154463-88154570 | + | 120 | 108 | SLITRK5 | Distal | 170301 | Not documented | 41 |
| L1M3b_dup490 | Uniquely | No | — | — | LINE | Other L1 | chr13: 92997670-92999999 | + | 130 | 2330 | GPC5 | Proximal | 0 | Not documented | 42 |
| L1PA3_dup9027 | Uniquely | No | — | — | LINE | L1PA\|B\|x | chr13: 94015384-94016476 | − | 10 | 1093 | GPC6 | Proximal | | Not documented | 43 |
| L1ME1_dup25834 | Multi | No | — | — | LINE | Other L1 | chr14: 102336289-102337224 | − | 110 | 936 | PPP2R5C | Proximal | 0 | Not documented | 44 |
| AluSx_dup103372 | Multi | No | — | — | SINE | Alu | chr14: 103799238-103799536 | − | 34 | 299 | EIF5 | Proximal | 346 | Not documented | 45 |
| AluSz6_dup34235 | Multi | No | — | — | SINE | Alu | chr14: 104056923-104057209 | + | 52 | 287 | APOPT1 | Proximal | 0 | Not documented | 46 |
| MIRc_dup83543 | Multi | No | — | — | SINE | MIR | chr14: 104151256-104151413 | − | 120 | 158 | RP11-73M18.2 | Proximal | 0 | Not documented | 47 |
| MIRc_dup83554 | Multi | No | — | — | SINE | MIR | chr14: 104378469-104378709 | − | 120 | 241 | C14orf2 | Proximal | 0 | Not documented | 48 |
| LTR48B_dup810 | Multi/ | No | — | — | LTR | ERV1 | chr14: 21408815-21409393 | + | 110 | 579 | RNASE2 | Distal | 14219 | Not documented | 49 |
| | Uniquely | | | | | | | | | | | | | | |
| SVA_D_dup1014 | Multi | No | — | — | Other | SVA | chr14: 21656418-21657641 | + | 11 | 1224 | HNRNPC | Distal | 19655 | Not documented | 1850 |
| AluSx1_dup77097 | Multi | No | — | — | SINE | Alu | chr14: 21965662-21965970 | − | 32 | 309 | TOX4 | Proximal | 0 | Not documented | 51 |
| AluJo_dup50827 | Multi | No | — | — | SINE | Alu | chr14: 21966008-21966126 | − | 73 | 119 | TOX4 | Proximal | 0 | Not documented | 52 |
| MIR3_dup70217 | Multi | No | — | — | SINE | MIR | chr14: 23080381-23080535 | − | 110 | 155 | ABHD4 | Proximal | 0 | Not documented | 53 |
| AluSx3_dup21042 | Uniquely | No | — | — | SINE | Alu | chr14: 23349301-23349601 | + | 51 | 301 | LRP10 | Proximal | 0 | Not documented | 54 |
| L4_dup13732 | Multi | No | — | — | LINE | RTE | chr14: 23732548-23732613 | − | 140 | 66 | C14orf164 | Proximal | 0 | Not documented | 55 |
| AluSp_dup35092 | Multi | No | — | — | SINE | Alu | chr14: 24409561-24409855 | − | 39 | 295 | DHRS4 | Distal | 12941 | Not documented | 56 |
| MIR3_dup70273 | Multi | No | — | — | SINE | MIR | chr14: 24615007-24615198 | − | 140 | 192 | PSME2 | Proximal | 0 | Not documented | 57 |
| L4_dup13748 | Multi | No | — | — | LINE | RTE | chr14: 25735703-25735768 | − | 140 | 66 | STXBP6 | Distal | 216201 | Not documented | 58 |
| L2a_dup134119 | Multi | No | — | — | LINE | L2 | chr14: 26934962-26935568 | − | 130 | 607 | NOVA1 | Proximal | 0 | Not documented | 59 |
| MLT1A1_dup5504 | Multi | No | — | — | LTR | ERVL- | chr14: 26935839-26936212 | + | 72 | 374 | NOVA1 | Proximal | 0 | Not documented | 60 |
| | | | | | | MaLR | | | | | | | | | |
| L2a_dup134121 | Multi | No | — | — | LINE | L2 | chr14: 26936282-26937114 | − | 120 | 833 | NOVA1 | Proximal | 0 | Not documented | 61 |
| MLT1E1A_dup2751 | Multi | No | — | — | LTR | ERVL- | chr14: 26937115-26937780 | + | 110 | 666 | NOVA1 | Proximal | 0 | Not documented | 62 |
| | | | | | | MaLR | | | | | | | | | |
| L2c_dup113283 | Uniquely | No | — | — | LINE | L2 | chr14: 26969236-26969510 | + | 140 | 275 | NOVA1 | Proximal | 0 | Not documented | 63 |
| L1MC2_dup4997 | Uniquely | No | — | — | LINE | Other L1 | chr14: 27026879-27027713 | + | 82 | 835 | NOVA1 | Proximal | 0 | Not documented | 64 |
| AluSc_dup25323 | Multi/ | No | — | — | SINE | Alu | chr14: 27048257-27048560 | − | 45 | 304 | NOVA1 | Proximal | 0 | Not documented | 65 |
| | Uniquely | | | | | | | | | | | | | | |
| L1PA13_dup7538 | Uniquely | No | — | — | LINE | L1PA\|B\|x | chr14: 27970139-27976798 | − | 69 | 6660 | NOVA1 | Distal | 903180 | Not documented | 66 |
| AluY_dup87061 | Uniquely | No | — | — | SINE | Alu | chr14: 29282148-29282456 | − | 44 | 309 | C14orf23 | Proximal | 0 | Not documented | 67 |
| THE1A-int_dup1233 | Uniquely | No | — | — | LTR | ERVL- | chr14: 30348771-30350329 | + | 45 | 1559 | PRKD1 | Proximal | 0 | Not documented | 68 |
| | | | | | | MaLR | | | | | | | | | |
| L1ME4a_dup33714 | Multi | No | — | — | LINE | Other L1 | chr14: 32740652-32741000 | + | 110 | 349 | AKAP6 | Distal | 57480 | Not documented | 69 |
| AluJo_dup51084 | Multi/ | No | — | — | SINE | Alu | chr14: 33422006-33422284 | + | 72 | 279 | NPAS3 | Proximal | 0 | Not documented | 70 |
| | Uniquely | | | | | | | | | | | | | | |
| LTR40b_dup982 | Uniquely | No | — | — | LTR | ERVL | chr14: 33422784-33423212 | − | 99 | 429 | NPAS3 | Proximal | 0 | Not documented | 71 |
| AluY_dup87235 | Uniquely | No | — | — | SINE | Alu | chr14: 33426573-33426877 | − | 28 | 305 | NPAS3 | Proximal | 0 | Not documented | 72 |
| FLAM_A_dup12157 | Multi | No | — | — | SINE | AIL | chr14: 33432077-33432179 | − | 80 | 103 | NPAS3 | Proximal | 0 | Not documented | 73 |
| MIRc_dup80837 | Multi | No | — | — | SINE | MIR | chr14: 33432957-33433023 | + | 140 | 67 | NPAS3 | Proximal | 0 | Not documented | 74 |
| AluJr_dup55200 | Multi | No | — | — | SINE | Alu | chr14: 33433899-33434196 | + | 56 | 298 | NPAS3 | Proximal | 0 | Not documented | 1875 |
| L1MA6_dup4618 | Multi/ | No | — | — | LINE | Other L1 | chr14: 33434214-33435794 | + | 62 | 1581 | NPAS3 | Proximal | 0 | Not documented | 76 |
| | Uniquely | | | | | | | | | | | | | | |
| Tigger3b_dup5394 | Multi | No | — | — | DNA | DNA | chr14: 33473389-33474584 | − | 63 | 1196 | NPAS3 | Proximal | 0 | Not documented | 77 |
| L3_dup36621 | Uniquely | No | — | — | LINE | CR1 | chr14: 33484230-33484457 | + | 160 | 228 | NPAS3 | Proximal | 0 | Not documented | 78 |
| L3_dup36623 | Multi | No | — | — | LINE | CR1 | chr14: 33485652-33485734 | + | 100 | 83 | NPAS3 | Proximal | 0 | Not documented | 79 |
| MIR_dup135997 | Uniquely | No | — | — | SINE | MIR | chr14: 33538690-33538939 | + | 130 | 250 | NPAS3 | Proximal | 0 | Not documented | 80 |
| Tigger1_dup9828 | Uniquely | No | — | — | DNA | DNA | chr14: 33639334-33641530 | − | 64 | 2197 | NPAS3 | Proximal | 0 | Not documented | 81 |
| L2_dup45790 | Uniquely | No | — | — | LINE | L2 | chr14: 33652770-33653051 | + | 160 | 282 | NPAS3 | Proximal | 0 | Not documented | 82 |
| L2b_dup74840 | Multi | No | — | — | LINE | L2 | chr14: 33708491-33708533 | − | 95 | 43 | NPAS3 | Proximal | 0 | Not documented | 83 |
| MIRb_dup174678 | Multi/ | No | — | — | SINE | MIR | chr14: 33715232-33715441 | + | 150 | 210 | NPAS3 | Proximal | 0 | Not documented | 84 |
| | Uniquely | | | | | | | | | | | | | | |
| AluYf4_dup1018 | Multi/ | No | — | — | SINE | Alu | chr14: 33715464-33715769 | + | 24 | 306 | NPAS3 | Proximal | 0 | Not documented | 85 |
| | Uniquely | | | | | | | | | | | | | | |
| AluSx_dup99914 | Multi | No | — | — | SINE | Alu | chr14: 33715917-33716264 | + | 70 | 348 | NPAS3 | Proximal | 0 | Not documented | 86 |
| L2c_dup113620 | Multi/ | No | — | — | LINE | L2 | chr14: 33730241-33730373 | + | 190 | 133 | NPAS3 | Proximal | 0 | Not documented | 87 |
| | Uniquely | | | | | | | | | | | | | | |
| L2a_dup134471 | Uniquely | No | — | — | LINE | L2 | chr14: 33746582-33747737 | − | 130 | 1156 | NPAS3 | Proximal | 0 | Not documented | 88 |
| MER5A_dup27199 | Multi/ | No | — | — | DNA | DNA | chr14: 33837561-33837739 | + | 130 | 179 | NPAS3 | Proximal | 0 | Not documented | 89 |
| | Uniquely | | | | | | | | | | | | | | |
| MER103C_dup7287 | Multi | No | — | — | DNA | DNA | chr14: 33837814-33837881 | − | 150 | 68 | NPAS3 | Proximal | 0 | Not documented | 90 |
| L3_dup36631 | Multi | No | — | — | LINE | CR1 | chr14: 33838236-33838760 | + | 150 | 525 | NPAS3 | Proximal | 0 | Not documented | 91 |
| L2c_dup113628 | Uniquely | No | — | — | LINE | L2 | chr14: 33839428-33840354 | − | 160 | 927 | NPAS3 | Proximal | 0 | Not documented | 92 |
| AluJb_dup102490 | Multi/ | No | — | — | SINE | Alu | chr14: 33852018-33852319 | + | 83 | 302 | NPAS3 | Proximal | 0 | Not documented | 93 |
| | Uniquely | | | | | | | | | | | | | | |
| AluSp_dup35187 | Multi | No | — | — | SINE | Alu | chr14: 33860394-33860697 | − | 39 | 304 | NPAS3 | Proximal | 0 | Not documented | 94 |
| MIRb_dup174699 | Multi | No | — | — | SINE | MIR | chr14: 33861481-33861672 | − | 150 | 192 | NPAS3 | Proximal | 0 | Not documented | 95 |
| L1PA7_dup11252 | Uniquely | No | — | — | LINE | L1PA\|B\|x | chr14: 33965068-33968053 | + | 33 | 2986 | NPAS3 | Proximal | 0 | Not documented | 96 |
| L1ME1_dup24985 | Multi | No | — | — | LINE | Other L1 | chr14: 34072910-34074370 | + | 110 | 1461 | NPAS3 | Proximal | 0 | Not documented | 97 |
| L1MEf_dup10976 | Uniquely | No | — | — | LINE | Other L1 | chr14: 36574240-36578026 | + | 110 | 3787 | BRMS1L | Distal | 172710 | Not documented | 98 |
| L2a_dup134632 | Multi | No | — | — | LINE | L2 | chr14: 36767852-36767912 | − | 120 | 61 | MBIP | Proximal | 0 | Not documented | 99 |
| AluSx_dup100607 | Multi | No | — | — | SINE | Alu | chr14: 50792667-50792966 | + | 44 | 300 | ATP5S | Proximal | 0 | Not documented | 1900 |
| MLT1F_dup3285 | Uniquely | No | — | — | LTR | ERVL- | chr14: 51203720-51204222 | + | 110 | 503 | NIN | Proximal | 0 | Not documented | 1 |
| | | | | | | MaLR | | | | | | | | | |
| L2b_dup75210 | Multi/ | No | — | — | LINE | L2 | chr14: 51443048-51443283 | + | 110 | 236 | TRIM9 | Proximal | 0 | Not documented | 2 |
| | Uniquely | | | | | | | | | | | | | | |
| L3_dup36895 | Multi | No | — | — | LINE | CR1 | chr14: 51455602-51455926 | − | 150 | 325 | TRIM9 | Proximal | 0 | Not documented | 3 |
| AluY_dup87893 | Uniquely | No | — | — | SINE | Alu | chr14: 51467947-51468246 | − | 17 | 300 | TRIM9 | Proximal | 0 | Not documented | 4 |
| AluY_dup87894 | Multi/ | No | — | — | SINE | Alu | chr14: 51468845-51469150 | − | 37 | 30€ | TRIM9 | Proximal | 0 | Not documented | 5 |
| | Uniquely | | | | | | | | | | | | | | |
| MIR_dup136790 | Uniquely | No | — | — | SINE | MIR | chr14: 51471307-51471527 | − | 120 | 221 | TRIM9 | Proximal | 0 | Not documented | 6 |
| L1MB3_dup12928 | Uniquely | No | — | — | LINE | Other L1 | chr14: 51478778-51480216 | − | 75 | 1439 | TRIM9 | Proximal | 0 | Not documented | 7 |
| L1MEf_dup11056 | Uniquely | No | — | — | LINE | Other L1 | chr14: 51529411-51531214 | + | 120 | 1804 | TRIM9 | Proximal | 0 | Not documented | 8 |
| Tigger4a_dup2727 | Multi | No | — | — | DNA | DNA | chr14: 51537181-51537402 | − | 75 | 222 | TRIM9 | Proximal | 0 | Not documented | 9 |
| Charlie8_dup2233 | Multi | No | — | — | DNA | DNA | chr14: 51537430-51537529 | + | 110 | 100 | TRIM9 | Proximal | 0 | Not documented | 10 |
| AluSp_dup35421 | Multi/ | No | — | — | SINE | Alu | chr14: 51537816-51538121 | + | 55 | 306 | TRIM9 | Proximal | 0 | Not documented | 11 |
| | Uniquely | | | | | | | | | | | | | | |
| L1ME3A_dup12518 | Multi/ | No | — | — | LINE | Other L1 | chr14: 51539721-51540515 | + | 100 | 795 | TRIM9 | Proximal | 0 | Not documented | 12 |
| | Uniquely | | | | | | | | | | | | | | |
| MIR_dup136798 | Uniquely | No | — | — | SINE | MIR | chr14: 51542755-51542963 | − | 110 | 209 | TRIM9 | Proximal | 0 | Not documented | 13 |
| L2a_dup135369 | Uniquely | No | — | — | LINE | L2 | chr14: 51559499-51559955 | − | 110 | 457 | TRIM9 | Proximal | 0 | Not documented | 14 |
| MER20_dup12212 | Uniquely | No | — | — | DNA | DNA | chr14: 53258925-53259155 | + | 100 | 231 | GNPNAT1 | Proximal | 540 | Not documented | 15 |
| L2_dup46196 | Uniquely | No | — | — | LINE | L2 | chr14: 53372151-53372538 | − | 150 | 388 | FERMT2 | Proximal | 0 | Not documented | 16 |
| MER20B_dup3255 | Multi/ | No | — | — | DNA | DNA | chr14: 55057953-55058588 | + | 120 | 636 | SAMD4A | Proximal | 0 | Not documented | 17 |
| | Uniquely | | | | | | | | | | | | | | |
| MLT1D_dup16011 | Uniquely | No | — | — | LTR | ERVL- | chr14: 55075746-55076238 | + | 88 | 493 | SAMD4A | Proximal | 0 | Not documented | 18 |
| | | | | | | MaLR | | | | | | | | | |
| AluSz_dup70406 | Multi | No | — | — | SINE | Alu | chr14: 55078313-55078623 | + | 50 | 311 | SAMD4A | Proximal | 0 | Not documented | 19 |
| Charlie1a_dup4583 | Uniquely | No | — | — | DNA | DNA | chr14: 55080621-55081146 | + | 73 | 526 | SAMD4A | Proximal | 0 | Not documented | 20 |
| MIRc_dup81321 | Multi/ | No | — | — | SINE | MIR | chr14: 55081901-55082029 | − | 120 | 129 | SAMD4A | Proximal | 0 | Not documented | 21 |
| | Uniquely | | | | | | | | | | | | | | |
| Tigger16b_dup898 | Multi/ | No | — | — | DNA | DNA | chr14: 55082137-55082382 | + | 130 | 246 | SAMD4A | Proximal | 0 | Not documented | 22 |
| | Uniquely | | | | | | | | | | | | | | |
| MER5B_dup19553 | Multi | No | — | — | DNA | DNA | chr14: 55089369-55089489 | − | 120 | 121 | SAMD4A | Proximal | 0 | Not documented | 23 |
| MER5A1_dup12242 | Multi | No | — | — | DNA | DNA | chr14: 55089490-55089646 | + | 68 | 157 | SAMD4A | Proximal | 0 | Not documented | 24 |
| L2b_dup75325 | Multi | No | — | — | LINE | L2 | chr14: 55089703-55089809 | + | 110 | 107 | SAMD4A | Proximal | 0 | Not documented | 1925 |
| MamRep605_dup3729 | Multi/ | No | — | — | Other | Other | chr14: 55089932-55090053 | − | 110 | 122 | SAMD4A | Proximal | 0 | Not documented | 26 |
| | Uniquely | | | | | Repeats | | | | | | | | | |
| L1MB3_dup12965 | Multi | No | — | — | LINE | Other L1 | chr14: 55096385-55097315 | − | 63 | 931 | SAMD4A | Proximal | 0 | Not documented | 27 |
| AluSx_dup100786 | Multi | No | — | — | SINE | Alu | chr14: 55102186-55102483 | + | 53 | 298 | SAMD4A | Proximal | 0 | Not documented | 28 |
| MLT1F1_dup2474 | Uniquely | No | — | — | LTR | ERVL- | chr14: 55164414-55164880 | − | 94 | 467 | SAMD4A | Proximal | 0 | Not documented | 29 |
| | | | | | | MaLR | | | | | | | | | |
| MER117_dup3525 | Multi | No | — | — | DNA | DNA | chr14: 57755885-57756055 | − | 160 | 171 | AP5M1 | Proximal | 0 | Not documented | 30 |
| AluJr_dup55758 | Multi | No | — | — | SINE | Alu | chr14: 58739682-58739889 | − | 75 | 208 | C14orf37 | Proximal | 0 | Not documented | 31 |
| AluSg_dup29906 | Multi | No | — | — | SINE | Alu | chr14: 58740242-58740533 | − | 56 | 292 | C14orf37 | Proximal | 0 | Not documented | 32 |
| L3_dup37000 | Multi | No | — | — | LINE | CR1 | chr14: 58890671-58890799 | − | 150 | 129 | TIMM9 | Proximal | 0 | Not documented | 33 |
| MIR3_dup70997 | Multi | No | — | — | SINE | MIR | chr14: 58890800-58890865 | + | 120 | 66 | TIMM9 | Proximal | 0 | Not documented | 34 |
| L1PA7_dup11443 | Uniquely | No | — | — | LINE | L1PA\|B\|x | chr14: 58991984-58997746 | + | 49 | 5763 | KIAA0586 | Proximal | 0 | EVE annotated | 35 |
| L2a_dup135883 | Multi | No | — | — | LINE | L2 | chr14: 59837028-59837159 | − | 86 | 132 | DAAM1 | Proximal | 0 | Not documented | 36 |
| LTR57_dup201 | Multi | No | — | — | LTR | ERVL | chr14: 59940842-59941240 | + | 68 | 399 | L3HYPDH | Proximal | 0 | Not documented | 37 |
| MIR_dup137344 | Multi/ | No | — | — | SINE | MIR | chr14: 59941287-59941546 | + | 130 | 260 | L3HYPDH | Proximal | 0 | Not documented | 38 |
| | Uniquely | | | | | | | | | | | | | | |
| Charlie1b_dup2447 | Multi | No | — | — | DNA | DNA | chr14: 59941948-59942429 | − | 71 | 482 | L3HYPDH | Proximal | 0 | Not documented | 39 |
| L4_dup14014 | Multi | No | — | — | LINE | RTE | chr14: 61438406-61438471 | − | 140 | 66 | TRMT5 | Proximal | 0 | Not documented | 40 |
| L1MB8_dup12938 | Uniquely | No | — | — | LINE | Other L1 | chr14: 63699950-63700182 | + | 81 | 233 | RHOJ | Proximal | 0 | Not documented | 41 |
| L2b_dup75617 | Uniquely | No | — | — | LINE | L2 | chr14: 63700408-63700702 | + | 160 | 295 | RHOJ | Proximal | 0 | Not documented | 42 |
| AluJr_dup55867 | Multi | No | — | — | SINE | Alu | chr14: 63759386-63759681 | + | 75 | 296 | RHOJ | Proximal | 0 | Not documented | 43 |
| Charlie24_dup1130 | Multi | No | — | — | DNA | DNA | chr14: 64882347-64882406 | + | 120 | 60 | MTHFD1 | Proximal | 0 | Not documented | 44 |
| L1MC3_dup10498 | Multi | No | — | — | LINE | Other L1 | chr14: 65399216-65400083 | + | 65 | 868 | CHURC1 | Proximal | 0 | Not documented | 45 |
| L3b_dup5875 | Multi | No | — | — | LINE | CR1 | chr14: 67831471-67831540 | + | 110 | 70 | EIF2S1 | Proximal | 0 | Not documented | 46 |
| AluJr4_dup13369 | Multi | No | — | — | SINE | Alu | chr14: 67852028-67852180 | + | 80 | 153 | EIF2S1 | Proximal | 0 | Not documented | 47 |
| FLAM_C_dup15985 | Multi | No | — | — | SINE | Alu | chr14: 67852379-67852490 | − | 69 | 112 | EIF2S1 | Proximal | 0 | Not documented | 48 |
| AluSq10_dup1684 | Multi | No | — | — | SINE | Alu | chr14: 68144497-68144578 | − | 39 | 82 | RDH11 | Proximal | 0 | Not documented | 49 |
| L1M2_dup7482 | Uniquely | No | — | — | LINE | Other L1 | chr14: 68398926-68400552 | − | 110 | 1627 | RAD51B | Proximal | 0 | Not documented | 1950 |
| AluSx1_dup78581 | Multi | No | — | — | SINE | Alu | chr14: 71051096-71051392 | − | 49 | 297 | MED6 | Proximal | 0 | Not documented | 51 |
| MER4E1_dup814 | Multi | No | — | — | LTR | ERV1 | chr14: 71979321-71979915 | + | 78 | 595 | SIPA1L1 | Proximal | 0 | Not documented | 52 |
| MIRb_dup177192 | Multi | No | — | — | SINE | MIR | chr14: 74178869-74178949 | − | 130 | 81 | PNMA1 | Proximal | 0 | Not documented | 53 |
| MamGyp-int_dup287 | Multi | No | — | — | LTR | Gypsy | chr14: 74179322-74180371 | − | 140 | 1050 | PNMA1 | Proximal | 0 | Not documented | 54 |
| AluSx_dup101827 | Multi | No | — | — | SINE | Alu | chr14: 74183096-74183412 | + | 42 | 317 | ELMSAN1 | Proximal | 0 | Not documented | 55 |
| AluSc8_dup15928 | Multi | No | — | — | SINE | Alu | chr14: 74524749-74525058 | + | 40 | 310 | ALDH6A1 | Proximal | 0 | Not documented | 56 |
| FLAM_C_dup16090 | Multi | No | — | — | SINE | Alu | chr14: 74526863-74527001 | − | 65 | 139 | ALDH6A1 | Proximal | 0 | Not documented | 57 |
| AluSc_dup25890 | Uniquely | No | — | — | SINE | Alu | chr14: 74666838-74667145 | + | 44 | 308 | LIN52 | Proximal | 0 | Not documented | 58 |
| AluSq10_dup1696 | Uniquely | No | — | — | SINE | Alu | chr14: 75180876-75181007 | + | 28 | 132 | FCF1 | Proximal | 0 | Not documented | 59 |
| L1MB7_dup17716 | Uniquely | No | — | — | LINE | Other L1 | chr14: 75526967-75527486 | + | 64 | 520 | ACYP1 | Proximal | 0 | Not documented | 60 |
| AluSp_dup35919 | Uniquely | No | — | — | SINE | Alu | chr14: 75528523-75528832 | + | 42 | 310 | ACYP1 | Proximal | 0 | Not documented | 61 |
| AluSz_dup71273 | Multi | No | — | — | SINE | Alu | chr14: 75598809-75599111 | + | 55 | 303 | TMED10 | Proximal | 0 | Not documented | 62 |
| L1MD2_dup8879 | Multi | No | — | — | LINE | Other L1 | chr14: 75599933-75600938 | + | 61 | 1006 | TMED10 | Proximal | 0 | Not documented | 63 |
| L1ME3B_dup6597 | Multi | No | — | — | LINE | Other L1 | chr14: 76117589-76117772 | + | 100 | 184 | C14orf1 | Proximal | 0 | Not documented | 64 |
| AluSq2_dup40417 | Multi | No | — | — | SINE | Alu | chr14: 77582033-77582327 | − | 39 | 295 | KIAA1737 | Proximal | 0 | Not documented | 65 |
| L1ME1_dup25536 | Multi | No | — | — | LINE | Other L1 | chr14: 78202927-78203314 | − | 98 | 388 | SNW1 | Proximal | 0 | Not documented | 66 |
| L4_dup14113 | Multi | No | — | — | LINE | RTE | chr14: 81289639-81289700 | + | 130 | 62 | CEP128 | Proximal | 0 | Not documented | 67 |
| MER30_dup3316 | Multi/ | No | — | — | DNA | DNA | chr14: 81729517-81729600 | + | 50 | 84 | STON2 | Proximal | 0 | Not documented | 68 |
| | Uniquely | | | | | | | | | | | | | | |
| AluSx_dup102333 | Multi/ | No | — | — | SINE | Alu | chr14: 81729601-81729911 | + | 47 | 311 | STON2 | Proximal | 0 | Not documented | 69 |
| | Uniquely | | | | | | | | | | | | | | |
| 7SK_dup611 | Multi | No | — | — | Other | Other | chr14: 90178855-90179094 | + | 7.7 | 240 | RP11-944C7.1 | Distal | 69841 | Not documented | 70 |
| | | | | | | Repeats | | | | | | | | | |
| AluSq2_dup40611 | Multi | No | — | — | SINE | Alu | chr14: 90266248-90266568 | + | 43 | 321 | EFCAB11 | Proximal | 0 | Not documented | 71 |
| AluY_dup89379 | Multi | No | — | — | SINE | Alu | chr14: 90523879-90524188 | + | 16 | 310 | KCNK13 | Distal | 3922 | Not documented | 72 |
| Tigger7_dup2559 | Multi | No | — | — | DNA | DNA | chr14: 91691582-91691985 | + | 90 | 404 | C14orf159 | Proximal | 0 | Not documented | 73 |
| AluSz_dup71901 | Multi | No | — | — | SINE | Alu | chr14: 93648993-93649289 | + | 54 | 297 | MOAP1 | Proximal | 0 | Not documented | 74 |
| MamGyp-int_dup288 | Multi | No | — | — | LTR | Gypsy | chr14: 93649573-93650621 | − | 140 | 1049 | MOAP1 | Proximal | 0 | Not documented | 1975 |
| AluSc_dup26136 | Uniquely | No | — | — | SINE | Alu | chr14: 95625029-95625327 | − | 40 | 299 | DICER1 | Proximal | 683 | Not documented | 76 |
| MLT1J_dup12397 | Uniquely | No | — | — | LTR | ERVL- | chr14: 95645938-95646294 | + | 130 | 357 | CLMN | Proximal | 1984 | Not documented | 77 |
| | | | | | | MaLR | | | | | | | | | |
| L2a_dup138222 | Multi/ | No | — | — | LINE | L2 | chr14: 96000020-96000082 | − | 94 | 63 | GLRX5 | Proximal | 0 | Not documented | 78 |
| | Uniquely | | | | | | | | | | | | | | |
| L2b_dup76867 | Multi | No | — | — | LINE | L2 | chr14: 96000283-96000343 | + | 100 | 61 | GLRX5 | Proximal | 0 | Not documented | 79 |
| FLAM_C_dup16263 | Multi/ | No | — | — | SINE | Alu | chr14: 96000438-96000606 | + | 40 | 169 | GLRX5 | Proximal | 0 | Not documented | 80 |
| | Uniquely | | | | | | | | | | | | | | |
| MIR3_dup72418 | Multi | No | — | — | SINE | MIR | chr14: 96553837-96553990 | − | 130 | 154 | C14orf132 | Proximal | 0 | Not documented | 81 |
| MIRb_dup179560 | Multi/ | No | — | — | SINE | MIR | chr14: 96555615-96555833 | − | 140 | 219 | C14orf132 | Proximal | 0 | Not documented | 82 |
| | Uniquely | | | | | | | | | | | | | | |
| AluSg_dup30507 | Multi | No | — | — | SINE | Alu | chr14: 96556456-96556757 | + | 51 | 302 | C14orf132 | Proximal | 0 | Not documented | 83 |
| MER20_dup12487 | Uniquely | No | — | — | DNA | DNA | chr14: 96559134-96559338 | + | 76 | 205 | C14orf132 | Proximal | 0 | Not documented | 84 |
| AluSx_dup107990 | Uniquely | No | — | — | SINE | Alu | chr15: 100231621-100231922 | + | 36 | 302 | MEF2A | Proximal | 0 | Not documented | 85 |
| AluY_dup90242 | Multi | No | — | — | SINE | Alu | chr15: 20682499-20682798 | + | 40 | 300 | GOLGA6L6 | Distal | 54297 | Not documented | 86 |
| AluY_dup90385 | Uniquely | No | — | — | SINE | Alu | chr15: 23086973-23087282 | + | 31 | 310 | NIPA1 | Proximal | 0 | Not documented | 87 |
| AluJb_dup106768 | Multi | No | — | — | SINE | Alu | chr15: 23296036-23296340 | + | 73 | 305 | GOLGA8I | Distal | 27214 | Not documented | 88 |
| AluY_dup90433 | Multi | No | — | — | SINE | Alu | chr15: 23349939-23350239 | + | 39 | 301 | RP11-467N20.5 | Distal | 54831 | Not documented | 89 |
| L2b_dup77458 | Multi | No | — | — | LINE | L2 | chr15: 25227628-25227733 | + | 130 | 106 | SNRPN | Distal | 3759 | Not documented | 90 |
| AluJr_dup57293 | Multi | No | — | — | SINE | Alu | chr15: 25227759-25228027 | − | 85 | 269 | SNRPN | Distal | 3890 | Not documented | 91 |
| AluSx1_dup80380 | Multi | No | — | — | SINE | Alu | chr15: 25228275-25228568 | + | 45 | 294 | SNRPN | Distal | 4406 | Not documented | 92 |
| AluSx_dup103886 | Multi | No | — | — | SINE | Alu | chr15: 25228657-25228952 | + | 65 | 296 | SNRPN | Distal | 4788 | Not documented | 93 |
| L1MDb_dup897 | Uniquely | No | — | — | LINE | Other L1 | chr15: 25341455-25342184 | − | 110 | 730 | SNRPN | Distal | 117586 | Not documented | 94 |
| AluSx1_dup80382 | Multi/ | No | — | — | SINE | Alu | chr15: 25345083-25345380 | − | 56 | 298 | SNRPN | Distal | 121214 | Not documented | 95 |
| | Uniquely | | | | | | | | | | | | | | |
| MER54A_dup506 | Multi/ | No | — | — | LTR | ERVL | chr15: 25360314-25360813 | + | 91 | 500 | SNRPN | Distal | 136445 | Not documented | 96 |
| | Uniquely | | | | | | | | | | | | | | |
| L1M5_dup51945 | Multi | No | — | — | LINE | Other L1 | chr15: 25364964-25365373 | | 120 | 410 | SNRPN | Distal | 141095 | Not documented | 97 |
| L1M4_dup14039 | Uniquely | No | — | — | LINE | Other L1 | chr15: 25578749-25579997 | + | 85 | 1249 | UBE3A | Distal | 2385 | Not documented | 98 |
| AluY_dup90788 | Multi | No | — | — | SINE | Alu | chr15: 28863181-28863480 | − | 40 | 300 | GOLGA8M | Distal | 83621 | Not documented | 99 |
| AluJb_dup107035 | Multi | No | — | — | SINE | Alu | chr15: 28916198-28916507 | − | 70 | 310 | GOLGA8M | Distal | 30594 | Not documented | 2000 |
| L4_dup14292 | Multi | No | — | — | LINE | RTE | chr15: 30023693-30023758 | + | 140 | 66 | TJP1 | Proximal | 0 | Not documented | 1 |
| L1ME1_dup26062 | Uniquely | No | — | — | LINE | Other L1 | chr15: 30090735-30091352 | + | 95 | 618 | TJP1 | Proximal | 0 | Not documented | 2 |
| L1PA12_dup1554 | Uniquely | No | — | — | LINE | L1PA\|B\|x | chr15: 33625220-33627911 | + | 41 | 2692 | RYR3 | Proximal | 0 | Not documented | 3 |
| HERVIP10F- | Uniquely | No | — | — | LTR | ERV1 | chr15: 34447433-34451570 | − | 93 | 4138 | KATNBL1 | Proximal | 0 | EVE annotated | 4 |
| int_dup336 | | | | | | | | | | | | | | | |
| AluSx1_dup80713 | Multi | No | — | — | SINE | Alu | chr15: 34829568-34829879 | + | 58 | 312 | GOLGA8B | Proximal | 0 | Not documented | 5 |
| MIRc_dup83960 | Uniquely | No | — | — | SINE | MIR | chr15: 36953310-36953385 | − | 150 | 76 | C15orf41 | Proximal | 0 | Not documented | 6 |
| MLT2B3_dup2842 | Uniquely | No | — | — | LTR | ERVL | chr15: 37273837-37274402 | + | 91 | 566 | MEIS2 | Proximal | 0 | Not documented | 7 |
| L1MA9_dup14286 | Uniquely | No | — | — | LINE | Other L1 | chr15: 37274403-37277259 | − | 66 | 2857 | MEIS2 | Proximal | 0 | Not documented | 8 |
| L1MD2_dup9103 | Uniquely | No | — | — | LINE | Other L1 | chr15: 38602642-38604206 | − | 89 | 1565 | SPRED1 | Proximal | 0 | Not documented | 9 |
| L1PA16_dup12253 | Multi | No | — | — | LINE | L1PA\|B\|x | chr15: 38610644-38611105 | − | 67 | 462 | SPRED1 | Proximal | 0 | Not documented | 10 |
| L4_dup14356 | Uniquely | No | — | — | LINE | RTE | chr15: 40685803-40685908 | − | 88 | 106 | KNSTRN | Proximal | 0 | Not documented | 11 |
| Tigger4b_dup2081 | Multi | No | — | — | DNA | DNA | chr15: 40710950-40711008 | + | 63 | 59 | IVD | Proximal | 0 | Not documented | 12 |
| AluSq2_dup41281 | Multi | No | — | — | SINE | Alu | chr15: 40711051-40711356 | + | 52 | 306 | IVD | Proximal | 0 | Not documented | 13 |
| AluSz_dup72878 | Multi | No | — | — | SINE | Alu | chr15: 40711428-40711726 | + | 61 | 299 | IVD | Proximal | 0 | Not documented | 14 |
| AluSx_dup104485 | Uniquely | No | — | — | SINE | Alu | chr15: 40986261-40986545 | − | 57 | 285 | RAD51 | Proximal | 428 | Not documented | 15 |
| LTR7_dup2081 | Multi | No | — | — | LTR | ERV1 | chr15: 41577519-41577884 | − | 42 | 366 | CHP1 | Distal | 3477 | Not documented | 16 |
| FLAM_A_dup12723 | Uniquely | No | — | — | SINE | Alu | chr15: 41673058-41673105 | − | 38 | 48 | NUSAP1 | Proximal | 0 | Not documented | 17 |
| AluSx1_dup80939 | Uniquely | No | — | — | SINE | Alu | chr15: 41898850-41899160 | − | 44 | 311 | MGA | Distal | 14263 | Not documented | 18 |
| AluSg_dup31036 | Uniquely | No | — | — | SINE | Alu | chr15: 42835068-42835366 | − | 52 | 299 | LRRC57 | Proximal | 0 | Not documented | 19 |
| AluSz_dup73054 | Uniquely | No | — | — | SINE | Alu | chr15: 42835397-42835698 | − | 53 | 302 | LRRC57 | Proximal | 0 | Not documented | 20 |
| L2_dup47422 | Uniquely | No | — | — | LINE | L2 | chr15: 43623863-43624423 | − | 170 | 561 | ADAL | Proximal | 0 | Not documented | 21 |
| L2c_dup118276 | Multi/ | No | — | — | LINE | L2 | chr15: 43695265-43695671 | − | 140 | 407 | TUBGCP4 | Proximal | 0 | Not documented | 22 |
| | Uniquely | | | | | | | | | | | | | | |
| MIR3_dup73386 | Multi | No | — | — | SINE | MIR | chr15: 43696803-43696885 | − | 130 | 83 | TUBGCP4 | Proximal | 0 | Not documented | 23 |
| MIR3_dup73387 | Multi | No | — | — | SINE | MIR | chr15: 43697006-43697110 | + | 130 | 105 | TUBGCP4 | Proximal | 0 | Not documented | 24 |
| L2a_dup139769 | Multi | No | — | — | LINE | L2 | chr15: 43697669-43697752 | − | 99 | 84 | TUBGCP4 | Proximal | 0 | Not documented | 2025 |
| AluSq2_dup41428 | Multi | No | — | — | SINE | Alu | chr15: 43697956-43698263 | + | 44 | 308 | TUBGCP4 | Proximal | 0 | Not documented | 26 |
| MIRb_dup181773 | Multi | No | — | — | SINE | MIR | chr15: 44088037-44088249 | + | 100 | 213 | SERINC4 | Proximal | 0 | Not documented | 27 |
| L2_dup47433 | Multi | No | — | — | LINE | L2 | chr15: 44097460-44097490 | − | 44 | 31 | MFAP1 | Proximal | 0 | Not documented | 28 |
| L1MA3_dup7828 | Uniquely | No | — | — | LINE | Other L1 | chr15: 44643797-44644828 | + | 49 | 1032 | CASC4 | Proximal | 0 | Not documented | 29 |
| AluSq2_dup41476 | Multi/ | No | — | — | SINE | Alu | chr15: 44706006-44706333 | − | 45 | 328 | CASC4 | Proximal | 0 | Not documented | 30 |
| | Uniquely | | | | | | | | | | | | | | |
| AluJo_dup53619 | Multi | No | — | — | SINE | Alu | chr15: 44827286-44827562 | − | 59 | 277 | EIF3J | Proximal | 1694 | Not documented | 31 |
| L2c_dup118338 | Multi | No | — | — | LINE | L2 | chr15: 45008992-45009130 | − | 160 | 139 | B2M | Proximal | 0 | Not documented | 32 |
| MIRc_dup84239 | Multi | No | — | — | SINE | MIR | chr15: 45009210-45009321 | − | 120 | 112 | B2M | Proximal | 0 | Not documented | 33 |
| Tigger3a_dup4527 | Multi | No | — | — | DNA | DNA | chr15: 45009328-45009601 | − | 45 | 274 | B2M | Proximal | 0 | Not documented | 34 |
| MLT1J_dup12525 | Multi | No | — | — | LTR | ERVL- | chr15: 45024682-45024732 | + | 140 | 51 | TRIM69 | Proximal | 0 | Not documented | 35 |
| | | | | | | MaLR | | | | | | | | | |
| MER49_dup1075 | Multi | No | — | — | LTR | ERV1 | chr15: 45024733-45025056 | + | 55 | 324 | TRIM69 | Proximal | 0 | Not documented | 36 |
| L1ME4a_dup35401 | Uniquely | No | — | — | LINE | Other L1 | chr15: 48449566-48449930 | + | 120 | 365 | MYEF2 | Proximal | 0 | Not documented | 37 |
| MamRep1161_{—} | Multi | No | — | — | DNA | DNA | chr15: 49171866-49172076 | − | 100 | 211 | EID1 | Proximal | 0 | Not documented | 38 |
| dup1535 | | | | | | | | | | | | | | | |
| L4_dup14399 | Multi | No | — | — | LINE | RTE | chr15: 49924335-49924395 | + | 140 | 61 | DTWD1 | Proximal | 0 | Not documented | 39 |
| AluSz_dup73376 | Uniquely | No | — | — | SINE | Alu | chr15: 50662519-50662814 | + | 60 | 296 | GABPB1 | Distal | 14915 | Not documented | 40 |
| AluSx_dup105196 | Multi/ | No | — | — | SINE | Alu | chr15: 50662901-50663188 | + | 71 | 288 | GABPB1 | Distal | 15297 | Not documented | 41 |
| | Uniquely | | | | | | | | | | | | | | |
| L1MC5_dup16569 | Multi | No | — | — | LINE | Other L1 | chr15: 50663217-50663393 | + | 95 | 177 | GABPB1 | Distal | 15613 | Not documented | 42 |
| L1M1_dup8394 | Multi | No | — | — | LINE | Other L1 | chr15: 52357479-52357560 | + | 110 | 82 | MAPK6 | Proximal | 0 | Not documented | 43 |
| Tigger7_dup2603 | Multi | No | — | — | DNA | DNA | chr15: 52840282-52840401 | + | 73 | 120 | ARPP19 | Proximal | 0 | Not documented | 44 |
| AluSx1_dup81462 | Multi | No | — | — | SINE | Alu | chr15: 52840402-52840710 | + | 40 | 309 | ARPP19 | Proximal | 0 | Not documented | 45 |
| Tigger7_dup2604 | Multi | No | — | — | DNA | DNA | chr15: 52840711-52840853 | + | 73 | 143 | ARPP19 | Proximal | 0 | Not documented | 46 |
| Tigger7_dup2605 | Multi | No | — | — | DNA | DNA | chr15: 52840852-52840950 | + | 64 | 99 | ARPP19 | Proximal | 0 | Not documented | 47 |
| MamRep434_dup1838 | Multi | No | — | — | DNA | DNA | chr15: 52840970-52841185 | − | 120 | 216 | ARPP19 | Proximal | 0 | Not documented | 48 |
| AluJo_dup53796 | Multi | No | — | — | SINE | Alu | chr15: 52842732-52842832 | + | 95 | 101 | ARPP19 | Proximal | 0 | Not documented | 49 |
| AluSg_dup31249 | Multi/ | No | — | — | SINE | Alu | chr15: 55609510-55609810 | − | 47 | 301 | RAB27A | Proximal | 0 | Not documented | 2050 |
| | Uniquely | | | | | | | | | | | | | | |
| L1P1_dup2775 | Uniquely | No | — | — | LINE | L1PA\|B\|x | chr15: 56728261-56731078 | + | 25 | 2818 | MNS1 | Proximal | 0 | EVE annotated | 51 |
| AluYa8_dup274 | Multi | No | — | — | SINE | Alu | chr15: 58007520-58007614 | − | 53 | 95 | POLR2M | Proximal | 0 | Not documented | 52 |
| L1ME4a_dup35586 | Multi | No | — | — | LINE | Other L1 | chr15: 58008270-58008406 | − | 100 | 137 | POLR2M | Proximal | 0 | Not documented | 53 |
| L1ME3B_dup6856 | Multi | No | — | — | LINE | Other L1 | chr15: 58009468-58010142 | − | 110 | 675 | POLR2M | Proximal | 0 | Not documented | 54 |
| L2b_dup78412 | Uniquely | No | — | — | LINE | L2 | chr15: 60958800-60959117 | − | 160 | 318 | RORA | Proximal | 0 | Not documented | 55 |
| UCON26_dup183 | Uniquely | No | — | — | Other | Other | chr15: 61152206-61152400 | + | 150 | 195 | RORA | Proximal | 0 | Not documented | 56 |
| | | | | | | Repeats | | | | | | | | | |
| MER5B_dup20347 | Uniquely | No | — | — | DNA | DNA | chr15: 61236347-61236511 | − | 100 | 165 | RORA | Proximal | 0 | Not documented | 57 |
| L1MB7_dup18362 | Uniquely | No | — | — | LINE | Other L1 | chr15: 61271959-61273178 | − | 76 | 1220 | RORA | Proximal | 0 | Not documented | 58 |
| L1MB7_dup18363 | Multi/ | No | — | — | LINE | Other L1 | chr15: 61273175-61275110 | − | 100 | 1936 | RORA | Proximal | 0 | Not documented | 59 |
| | Uniquely | | | | | | | | | | | | | | |
| AluSc_dup26809 | Uniquely | No | — | — | SINE | Alu | chr15: 61277782-61278078 | − | 43 | 297 | RORA | Proximal | 0 | Not documented | 60 |
| THE1A_dup3710 | Uniquely | No | — | — | LTR | ERVL- | chr15: 61315422-61315776 | − | 60 | 355 | RORA | Proximal | 0 | Not documented | 61 |
| | | | | | | MaLR | | | | | | | | | |
| L1MC4_dup23871 | Uniquely | No | — | — | LINE | Other L1 | chr15: 61322229-61322631 | + | 110 | 403 | RORA | Proximal | 0 | Not documented | 62 |
| MIRb_dup182948 | Uniquely | No | — | — | SINE | MIR | chr15: 61325109-61325333 | − | 130 | 225 | RORA | Proximal | 0 | Not documented | 63 |
| AluSc8_dup16512 | Multi | No | — | — | SINE | Alu | chr15: 61341849-61342157 | − | 53 | 309 | RORA | Proximal | 0 | Not documented | 64 |
| MER58A_dup10735 | Uniquely | No | — | — | DNA | DNA | chr15: 61342307-61342510 | + | 110 | 204 | RORA | Proximal | 0 | Not documented | 65 |
| Charlie15a_{—} | Uniquely | No | — | — | DNA | DNA | chr15: 61387844-61387941 | − | 120 | 98 | RORA | Proximal | 0 | Not documented | 66 |
| dup2850 | | | | | | | | | | | | | | | |
| AluSx_dup105811 | Uniquely | No | — | — | SINE | Alu | chr15: 61392307-61392618 | − | 52 | 312 | RORA | Proximal | 0 | Not documented | 67 |
| L2c_dup119188 | Uniquely | No | — | — | LINE | L2 | chr15: 61496614-61496877 | − | 150 | 264 | RORA | Proximal | 0 | Not documented | 68 |
| AluSx_dup105905 | Multi | No | — | — | SINE | Alu | chr15: 63616887-63616995 | − | 38 | 109 | CA12 | Proximal | 0 | Not documented | 69 |
| AluSx3_dup22530 | Uniquely | No | — | — | SINE | Alu | chr15: 65441535-65441829 | + | 59 | 295 | CLPX | Proximal | 0 | Not documented | 70 |
| AluJr_dup58497 | Multi | No | — | — | SINE | Alu | chr15: 65869492-65869764 | + | 67 | 273 | PTPLAD1 | Proximal | 0 | Not documented | 71 |
| AluSp_dup37548 | Uniquely | No | — | — | SINE | Alu | chr15: 66785711-66786022 | + | 54 | 312 | SNAPC5 | Proximal | 0 | Not documented | 72 |
| FLAM_A_dup12961 | Multi | No | — | — | SINE | Alu | chr15: 67811339-67811457 | + | 120 | 119 | C15orf61 | Proximal | 1950 | Not documented | 73 |
| MIRc_dup85478 | Multi | No | — | — | SINE | MIR | chr15: 69018927-69019165 | − | 150 | 239 | CORO2B | Proximal | 0 | Not documented | 74 |
| MIRc_dup85479 | Multi | No | — | — | SINE | MIR | chr15: 69019511-69019648 | − | 110 | 138 | CORO2B | Proximal | 0 | Not documented | 2075 |
| L2_dup47885 | Multi/ | No | — | — | LINE | L2 | chr15: 69952512-69954504 | − | 140 | 1993 | RPLP1 | Distal | 204258 | Not documented | 76 |
| | Uniquely | | | | | | | | | | | | | | |
| MIR3_dup75136 | Multi | No | — | — | SINE | MIR | chr15: 75931904-75931928 | + | 36 | 25 | IMP3 | Proximal | 0 | Not documented | 77 |
| AluSq2_dup42274 | Uniquely | No | — | — | SINE | Alu | chr15: 80303585-80303888 | + | 48 | 304 | BCL2A1 | Distal | 39798 | Not documented | 78 |
| L1MDb_dup917 | Multi | No | — | — | LINE | Other L1 | chr15: 80352106-80352300 | + | 140 | 195 | ZFAND6 | Proximal | 0 | Not documented | 79 |
| MIR3_dup75470 | Multi | No | — | — | SINE | MIR | chr15: 80887230-80887286 | + | 110 | 57 | ARNT2 | Proximal | 0 | Not documented | 80 |
| L2c_dup120664 | Multi/ | No | — | — | LINE | L2 | chr15: 83423430-83423513 | − | 120 | 84 | FSD2 | Proximal | 602 | Not documented | 81 |
| | Uniquely | | | | | | | | | | | | | | |
| AluSq2_dup42339 | Multi | No | — | — | SINE | Alu | chr15: 83674031-83674335 | − | 42 | 305 | C15orf40 | Proximal | 0 | Not documented | 82 |
| MIRb_dup185509 | Multi/ | No | — | — | SINE | MIR | chr15: 85186484-85186669 | − | 110 | 186 | WDR73 | Proximal | 0 | Not documented | 83 |
| | Uniquely | | | | | | | | | | | | | | |
| AluSx1_dup83210 | Multi | No | — | — | SINE | Alu | chr15: 90377849-90378161 | + | 48 | 313 | AP3S2 | Proximal | 0 | Not documented | 84 |
| AluSz_dup75373 | Multi/ | No | — | — | SINE | Alu | chr15: 93627690-93628000 | − | 60 | 311 | RGMA | Proximal | 0 | Not documented | 85 |
| | Uniquely | | | | | | | | | | | | | | |
| MLT1A_dup7615 | Uniquely | No | — | — | LTR | ERVL- | chr15: 99676572-99676916 | − | 82 | 345 | TTC23 | Proximal | 0 | Not documented | 86 |
| | | | | | | MaLR | | | | | | | | | |
| HAL1-3A_ME_{—} | Multi | No | — | — | LINE | Other L1 | chr16: 11931743-11931906 | − | 110 | 164 | RSL1D1 | Proximal | 0 | Not documented | 87 |
| dup1613 | | | | | | | | | | | | | | | |
| Tigger14a_dup1146 | Multi/ | No | — | — | DNA | DNA | chr16: 14356974-14357124 | + | 150 | 151 | MKL2 | Proximal | 0 | Not documented | 88 |
| | Uniquely | | | | | | | | | | | | | | |
| AluSp_dup38584 | Uniquely | No | — | — | SINE | Alu | chr16: 14708427-14708733 | + | 46 | 307 | PARN | Proximal | 0 | Not documented | 89 |
| L1ME4a_dup36952 | Multi | No | — | — | LINE | Other L1 | chr16: 14946155-14946316 | − | 150 | 162 | NOMO1 | Proximal | 0 | Not documented | 90 |
| L1ME4a_dup36983 | Multi | No | — | — | LINE | Other L1 | chr16: 15681838-15682223 | − | 120 | 386 | C16orf45 | Proximal | 0 | Not documented | 91 |
| AluSx_dup109754 | Multi | No | — | — | SINE | Alu | chr16: 15960783-15961078 | + | 52 | 296 | FOPNL | Proximal | 0 | Not documented | 92 |
| L1ME4a_dup36993 | Multi | No | — | — | LINE | Other L1 | chr16: 16344653-16344814 | − | 150 | 162 | NOMO3 | Proximal | 0 | Not documented | 93 |
| AluSx1_dup83683 | Multi | No | — | — | SINE | Alu | chr16: 174794-175096 | + | 51 | 303 | NPRL3 | Proximal | 0 | Not documented | 94 |
| L1ME4a_dup37048 | Multi | No | — | — | LINE | Other L1 | chr16: 18555062-18555223 | + | 150 | 162 | NOMO2 | Proximal | 0 | Not documented | 95 |
| AluY_dup94638 | Multi | No | — | — | SINE | Alu | chr16: 1878001-1878298 | + | 27 | 298 | FAHD1 | Proximal | 0 | Not documented | 96 |
| MIR3_dup76714 | Multi | No | — | — | SINE | MIR | chr16: 19513986-19514098 | + | 150 | 113 | GDE1 | Proximal | 0 | Not documented | 97 |
| AluSx1_dup85587 | Multi | No | — | — | SINE | Alu | chr16: 21830479-21830774 | − | 34 | 296 | NPIPB4 | Distal | 15117 | Not documented | 98 |
| AluSx_dup108289 | Multi | No | — | — | SINE | Alu | chr16: 2202183-2202388 | + | 49 | 206 | RAB26 | Proximal | 0 | Not documented | 99 |
| AluSx3_dup22980 | Multi | No | — | — | SINE | Alu | chr16: 2202391-2202723 | + | 50 | 333 | RAB26 | Proximal | 0 | Not documented | 2100 |
| MIRc_dup87132 | Multi | No | — | — | SINE | MIR | chr16: 2255832-2255973 | + | 130 | 142 | MLST8 | Proximal | 0 | Not documented | 1 |
| CR1_Mam_dup1564 | Multi | No | — | — | LINE | CR1 | chr16: 2326243-2326331 | − | 140 | 89 | ABCA3 | Proximal | 0 | Not documented | 2 |
| MLT1K_dup14797 | Multi | No | — | — | LTR | ERVL- | chr16: 23680568-23680704 | − | 90 | 137 | DCTN5 | Proximal | 0 | Not documented | 3 |
| | | | | | | MaLR | | | | | | | | | |
| MLT1K_dup14798 | Multi | No | — | — | LTR | ERVL- | chr16: 23680739-23680973 | − | 130 | 235 | DCTN5 | Proximal | 0 | Not documented | 4 |
| | | | | | | MaLR | | | | | | | | | |
| AluSz6_dup36598 | Uniquely | No | — | — | SINE | Alu | chr16: 24553778-24554066 | − | 52 | 289 | RBBP6 | Proximal | 0 | Not documented | 5 |
| AluJb_dup113864 | Multi | No | — | — | SINE | Alu | chr16: 24554614-24554923 | − | 65 | 310 | RBBP6 | Proximal | 0 | Not documented | 6 |
| L1PA16_dup12541 | Multi | No | — | — | LINE | L1PA\|B\|x | chr16: 24637332-24637504 | − | 68 | 173 | RBBP6 | Distal | 53149 | Not documented | 7 |
| LTR33_dup7910 | Uniquely | No | — | — | LTR | ERVL | chr16: 24693928-24694214 | − | 120 | 287 | TNRC6A | Distal | 46803 | Not documented | 8 |
| AluSz_dup77479 | Uniquely | No | — | — | SINE | Alu | chr16: 24744256-24744544 | + | 61 | 289 | TNRC6A | Proximal | 0 | Not documented | 9 |
| AluJr_dup61096 | Multi | No | — | — | SINE | Alu | chr16: 24745215-24745525 | + | 72 | 311 | TNRC6A | Proximal | 0 | Not documented | 10 |
| AluSz_dup77480 | Uniquely | No | — | — | SINE | Alu | chr16: 24746321-24746616 | + | 58 | 296 | TNRC6A | Proximal | 0 | Not documented | 11 |
| L2_dup48575 | Multi | No | — | — | LINE | L2 | chr16: 2945196-2945247 | + | 98 | 52 | FLYWCH2 | Proximal | 0 | Not documented | 12 |
| AluSz_dup77800 | Multi | No | — | — | SINE | Alu | chr16: 29467767-29468034 | − | 54 | 268 | SLX1B | Proximal | 0 | Not documented | 13 |
| AluSx1_dup86274 | Multi | No | — | — | SINE | Alu | chr16: 29468259-29468570 | − | 42 | 312 | SLX1B | Proximal | 0 | Not documented | 14 |
| L1ME3D_dup3961 | Multi | No | — | — | LINE | Other L1 | chr16: 29983100-29983427 | − | 140 | 328 | TMEM219 | Proximal | 0 | Not documented | 15 |
| AluSz_dup77864 | Multi | No | — | — | SINE | Alu | chr16: 30207109-30207376 | − | 54 | 268 | SLX1A | Proximal | 0 | Not documented | 16 |
| AluSx1_dup86390 | Multi | No | — | — | SINE | Alu | chr16: 30207599-30207912 | − | 44 | 314 | SLX1A | Proximal | 0 | Not documented | 17 |
| MIR3_dup76255 | Multi/ | No | — | — | SINE | MIR | chr16: 3022154-3022273 | + | 140 | 120 | PAQR4 | Proximal | 0 | Not documented | 18 |
| | Uniquely | | | | | | | | | | | | | | |
| MLT1D_dup17192 | Multi | No | — | — | LTR | ERVL- | chr16: 30362045-30362510 | − | 100 | 466 | CD2BP2 | Proximal | 0 | Not documented | 19 |
| | | | | | | MaLR | | | | | | | | | |
| MER104_dup1879 | Multi | No | — | — | DNA | DNA | chr16: 30362710-30362851 | + | 140 | 142 | CD2BP2 | Proximal | 0 | Not documented | 20 |
| MIR_dup148262 | Multi | No | — | — | SINE | MIR | chr16: 30434927-30435159 | − | 120 | 233 | ZNF771 | Proximal | 0 | Not documented | 21 |
| LTR83_dup630 | Multi/ | No | — | — | LTR | ERVL | chr16: 30457085-30457148 | − | 110 | 64 | SEPHS2 | Proximal | 0 | Not documented | 22 |
| | Uniquely | | | | | | | | | | | | | | |
| AluSp_dup39070 | Multi | No | — | — | SINE | Alu | chr16: 30646235-30646510 | + | 33 | 276 | ZNF689 | Distal | 10903 | Not documented | 23 |
| AluSp_dup38297 | Multi/ | No | — | — | SINE | Alu | chr16: 3074939-3075242 | − | 43 | 304 | THOC6 | Proximal | 0 | Not documented | 24 |
| | Uniquely | | | | | | | | | | | | | | |
| AluJr4_dup14258 | Multi | No | — | — | SINE | Alu | chr16: 3075248-3075353 | − | 56 | 106 | THOC6 | Proximal | 0 | Not documented | 2125 |
| SVA_D_dup1100 | Multi | No | — | — | Other | SVA | chr16: 308155-308770 | − | 9.5 | 616 | ITFG3 | Proximal | 0 | Not documented | 26 |
| L1ME3A_dup13428 | Uniquely | No | — | — | LINE | Other L1 | chr16: 30905895-30906504 | + | 110 | 610 | BCL7C | Proximal | 0 | Not documented | 27 |
| AluJo_dup57062 | Multi | No | — | — | SINE | Alu | chr16: 30999655-30999951 | − | 63 | 297 | HSD3B7 | Proximal | 0 | Not documented | 28 |
| LTR41_dup1506 | Multi/ | No | — | — | LTR | ERVL | chr16: 3179171-3179829 | − | 90 | 659 | ZNF213 | Proximal | 0 | Not documented | 29 |
| | Uniquely | | | | | | | | | | | | | | |
| REP522_dup147 | Multi | No | — | — | Other | Other | chr16: 33312707-33312895 | − | 87 | 189 | TP53TG3C | Distal | 47981 | Not documented | 30 |
| | | | | | | Repeats | | | | | | | | | |
| AluJr_dup61722 | Multi | No | — | — | SINE | Alu | chr16: 33388630-33388929 | + | 80 | 300 | TP53TG3C | Distal | 123904 | Not documented | 31 |
| AluJb_dup114852 | Multi | No | — | — | SINE | Alu | chr16: 33389144-33389355 | + | 86 | 212 | TP53TG3C | Distal | 124418 | Not documented | 32 |
| TAR1_dup131 | Multi | No | — | — | Other | Other | chr16: 33509673-33510071 | + | 73 | 399 | RP11-812E19.9 | Distal | 136974 | Not documented | 33 |
| | | | | | | Repeats | | | | | | | | | |
| AluSx1_dup83938 | Multi | No | — | — | SINE | Alu | chr16: 3359175-3359479 | + | 64 | 305 | ZNF75A | Proximal | 0 | Not documented | 34 |
| MLT2C1_dup2235 | Multi | No | — | — | LTR | ERVL | chr16: 33646016-33646179 | + | 170 | 164 | RP11-812E19.9 | Proximal | 866 | Not documented | 35 |
| L3b_dup6163 | Multi | No | — | — | LINE | CR1 | chr16: 3368474-3368560 | + | 100 | 87 | ZNF75A | Proximal | 0 | Not documented | 36 |
| AluSz_dup78056 | Multi | No | — | — | SINE | Alu | chr16: 33702657-33702956 | − | 53 | 300 | RP11-812E19.9 | Distal | 54962 | Not documented | 37 |
| AluJo_dup57163 | Multi | No | — | — | SINE | Alu | chr16: 33703299-33703595 | + | 61 | 297 | RP11-812E19.9 | Distal | 55604 | Not documented | 38 |
| L1ME1_dup27179 | Multi | No | — | — | LINE | Other L1 | chr16: 33742304-33742489 | − | 110 | 186 | RP11-812E19.9 | Distal | 94609 | Not documented | 39 |
| AluSg7_dup6598 | Multi | No | — | — | SINE | Alu | chr16: 33761080-33761171 | − | 50 | 92 | RP11-812E19.9 | Distal | 113385 | Not documented | 40 |
| MIR_dup148402 | Multi | No | — | — | SINE | MIR | chr16: 33780697-33780770 | − | 110 | 74 | RP11-812E19.9 | Distal | 133002 | Not documented | 41 |
| L1PB4_dup6644 | Multi | No | — | — | LINE | L1PA\|B\|x | chr16: 33813669-33814440 | − | 91 | 772 | RP11-812E19.9 | Distal | 165974 | Not documented | 42 |
| MLT1A0_dup17950 | Multi | No | — | — | LTR | ERVL- | chr16: 33843762-33844055 | − | 110 | 294 | RP11-812E19.9 | Distal | 196067 | Not documented | 43 |
| | | | | | | MaLR | | | | | | | | | |
| HSATII_dup243 | Multi | No | — | — | Other | Other | chr16: 33888764-33888852 | + | 46 | 89 | RP11-812E19.9 | Distal | 241069 | Not documented | 44 |
| | | | | | | Repeats | | | | | | | | | |
| MER39B_dup1051 | Multi | No | — | — | LTR | ERV1 | chr16: 33955785-33955914 | + | 100 | 130 | CTD-2144E22.5 | Distal | 300594 | Not documented | 45 |
| MER45C_dup1011 | Multi | No | — | — | DNA | DNA | chr16: 33964922-33965030 | + | 88 | 109 | CTD-2144E22.5 | Distal | 291478 | Not documented | 46 |
| HSATII_dup271 | Multi | No | — | — | Other | Other | chr16: 34185051-34187438 | + | 56 | 2388 | CTD-2144E22.5 | Distal | 69070 | Not documented | 47 |
| | | | | | | Repeats | | | | | | | | | |
| L1PB_dup1519 | Multi | No | — | — | LINE | L1PA\|B\|x | chr16: 34215663-34216882 | + | 36 | 1220 | CTD-2144E22.5 | Distal | 39626 | Not documented | 48 |
| MER57A-int_{—} | Multi | No | — | — | LTR | ERV1 | chr16: 34218447-34219679 | + | 80 | 1233 | CTD-2144E22.5 | Distal | 36829 | Not documented | 49 |
| dup1548 | | | | | | | | | | | | | | | |
| AluSx1_dup86647 | Multi | No | — | — | SINE | Alu | chr16: 34223357-34223650 | − | 63 | 294 | CTD-2144E22.5 | Distal | 32858 | Not documented | 2150 |
| L1P1_dup2837 | Multi | No | — | — | LINE | L1PA\|B\|x | chr16: 34229376-34231396 | + | 32 | 2021 | CTD-2144E22.5 | Distal | 25112 | EVE annotated | 51 |
| HERVK-int_dup206 | Multi | No | — | — | LTR | ERVK | chr16: 34231474-34233184 | + | 5.9 | 1711 | CTD-2144E22.5 | Distal | 23324 | EVE annotated | 52 |
| AluSc8_dup17407 | Multi | No | — | — | SINE | Alu | chr16: 34264170-34264477 | − | 46 | 308 | CTD-2144E22.5 | Distal | 6893 | Not documented | 53 |
| THE1B_dup19815 | Multi | No | — | — | LTR | ERVL- | chr16: 34474626-34474793 | + | 73 | 168 | CTD-2144E22.5 | Distal | 217349 | Not documented | 54 |
| | | | | | | MaLR | | | | | | | | | |
| HERVS71-int_{—} | Multi | No | — | — | LTR | ERV1 | chr16: 34503416-34510453 | − | 85 | 7038 | CTD-2144E22.5 | Distal | 246139 | EVE annotated | 55 |
| dup121 | | | | | | | | | | | | | | | |
| L4_dup14856 | Multi | No | — | — | LINE | RTE | chr16: 35036713-35036778 | − | 140 | 66 | CTD-2144E22.5 | Distal | 779436 | Not documented | 56 |
| AluSx_dup111737 | Uniquely | No | — | — | SINE | Alu | chr16: 46731745-46732034 | + | 45 | 290 | ORC6 | Proximal | 0 | Not documented | 57 |
| LTR12C_dup2432 | Multi | No | — | — | LTR | ERV1 | chr16: 47177625-47177852 | + | 57 | 228 | NETO2 | Proximal | 0 | Not documented | 58 |
| MIRb_dup191533 | Multi | No | — | — | SINE | MIR | chr16: 47188905-47189159 | − | 130 | 255 | ITFG1 | Proximal | 0 | Not documented | 59 |
| MIRb_dup191622 | Multi | No | — | — | SINE | MIR | chr16: 48386861-48387015 | + | 130 | 155 | LONP2 | Proximal | 0 | Not documented | 60 |
| MER3_dup9090 | Multi | No | — | — | DNA | DNA | chr16: 54146246-54146448 | − | 72 | 203 | FTO | Proximal | 0 | Not documented | 61 |
| MIR_dup149081 | Multi | No | — | — | SINE | MIR | chr16: 54146835-54147052 | + | 100 | 218 | FTO | Proximal | 0 | Not documented | 62 |
| MIRb_dup192212 | Multi | No | — | — | SINE | MIR | chr16: 54147429-54147552 | + | 140 | 124 | FTO | Proximal | 0 | Not documented | 63 |
| MER102b_dup3456 | Multi | No | — | — | DNA | DNA | chr16: 54147568-54147708 | + | 120 | 141 | FTO | Proximal | 0 | Not documented | 64 |
| AluSc_dup28075 | Multi | No | — | — | SINE | Alu | chr16: 54147709-54148015 | − | 43 | 307 | FTO | Proximal | 0 | Not documented | 65 |
| MER102b_dup3457 | Multi | No | — | — | DNA | DNA | chr16: 54148016-54148205 | + | 130 | 190 | FTO | Proximal | 0 | Not documented | 66 |
| L1MB3_dup14285 | Multi | No | — | — | LINE | Other L1 | chr16: 56510399-56510718 | − | 63 | 320 | OGFOD1 | Proximal | 0 | Not documented | 67 |
| L1MC4a_dup23272 | Multi | No | — | — | LINE | Other L1 | chr16: 56535825-56536024 | − | 120 | 200 | BBS2 | Proximal | 0 | Not documented | 68 |
| AluSz_dup78487 | Uniquely | No | — | — | SINE | Alu | chr16: 56543135-56543437 | + | 57 | 303 | BBS2 | Proximal | 0 | Not documented | 69 |
| L4_dup14913 | Multi/ | No | — | — | LINE | RTE | chr16: 57673541-57673585 | − | 120 | 45 | GPR56 | Proximal | 0 | Not documented | 70 |
| | Uniquely | | | | | | | | | | | | | | |
| AluSz6_dup37069 | Multi/ | No | — | — | SINE | Alu | chr16: 57675478-57675776 | − | 56 | 299 | GPR56 | Proximal | 0 | Not documented | 71 |
| | Uniquely | | | | | | | | | | | | | | |
| L2b_dup82597 | Multi/ | No | — | — | LINE | L2 | chr16: 57698574-57698807 | − | 160 | 234 | GPR56 | Proximal | 0 | Not documented | 72 |
| | Uniquely | | | | | | | | | | | | | | |
| L1ME4a_dup37483 | Multi | No | — | — | LINE | Other L1 | chr16: 57698830-57698993 | − | 130 | 164 | GPR56 | Proximal | 0 | Not documented | 73 |
| Tigger3b_dup5790 | Uniquely | No | — | — | DNA | DNA | chr16: 58158287-58159306 | − | 57 | 1020 | C16orf80 | Proximal | 0 | Not documented | 74 |
| L1PA10_dup6245 | Uniquely | No | — | — | LINE | L1PA\|B\|x | chr16: 58363750-58364460 | − | 36 | 711 | GINS3 | Proximal | 0 | Not documented | 2175 |
| L1PA14_dup2804 | Multi | No | — | — | LINE | L1PA\|B\|x | chr16: 61984605-61984964 | + | 60 | 360 | CDH8 | Proximal | 0 | Not documented | 76 |
| L2c_dup124027 | Uniquely | No | — | — | LINE | L2 | chr16: 65065289-65065503 | − | 160 | 215 | CDH11 | Proximal | 0 | Not documented | 77 |
| FLAM_A_dup13509 | Multi | No | — | — | SINE | Alu | chr16: 66978093-66978226 | + | 88 | 134 | CES2 | Proximal | 0 | Not documented | 78 |
| FLAM_C_dup17871 | Multi | No | — | — | SINE | Alu | chr16: 67133319-67133436 | − | 42 | 118 | CBFB | Proximal | 0 | Not documented | 79 |
| L2c_dup124153 | Multi | No | — | — | LINE | L2 | chr16: 67209504-67209581 | + | 140 | 78 | NOL3 | Proximal | 0 | Not documented | 80 |
| MIRc_dup89501 | Multi | No | — | — | SINE | MIR | chr16: 69376035-69376132 | + | 150 | 98 | NIP7 | Proximal | 0 | Not documented | 81 |
| AluSz_dup79074 | Multi | No | — | — | SINE | Alu | chr16: 69376216-69376497 | + | 63 | 282 | NIP7 | Proximal | 0 | Not documented | 82 |
| AluY_dup97426 | Multi/ | No | — | — | SINE | Alu | chr16: 69390423-69390732 | + | 25 | 310 | TERF2 | Proximal | 0 | Not documented | 83 |
| | Uniquely | | | | | | | | | | | | | | |
| AluJr_dup62728 | Multi | No | — | — | SINE | Alu | chr16: 69496865-69496990 | + | 58 | 126 | CYB5B | Proximal | 0 | Not documented | 84 |
| AluSz_dup79159 | Multi | No | — | — | SINE | Alu | chr16: 70281676-70281979 | + | 66 | 304 | EXOSC6 | Distal | 2156 | Not documented | 85 |
| AluJb_dup116565 | Multi | No | — | — | SINE | Alu | chr16: 70282505-70282816 | − | 75 | 312 | EXOSC6 | Proximal | 1319 | Not documented | 86 |
| AluSz_dup79160 | Multi | No | — | — | SINE | Alu | chr16: 70283020-70283314 | + | 60 | 295 | EXOSC6 | Proximal | 821 | Not documented | 87 |
| AluSz_dup79172 | Multi | No | — | — | SINE | Alu | chr16: 70406854-70407148 | + | 52 | 295 | DDX19A | Proximal | 0 | Not documented | 88 |
| AluSx_dup113205 | Multi | No | — | — | SINE | Alu | chr16: 70412069-70412326 | + | 39 | 258 | ST3GAL2 | Proximal | 1013 | Not documented | 89 |
| L3_dup39872 | Multi | No | — | — | LINE | CR1 | chr16: 70548206-70548270 | − | 150 | 65 | COG4 | Proximal | 0 | Not documented | 90 |
| L2_dup49541 | Multi | No | — | — | LINE | L2 | chr16: 70699682-70700081 | − | 150 | 400 | MTSS1L | Proximal | 0 | Not documented | 91 |
| MLT1A0_dup18127 | Uniquely | No | — | — | LTR | ERVL- | chr16: 70702491-70702836 | + | 87 | 346 | MTSS1L | Proximal | 0 | Not documented | 92 |
| | | | | | | MaLR | | | | | | | | | |
| L1ME3A_dup13644 | Uniquely | No | — | — | LINE | Other L1 | chr16: 70702837-70703304 | + | 100 | 468 | MTSS1L | Proximal | 0 | Not documented | 93 |
| L1MC4a_dup23464 | Multi/ | No | — | — | LINE | Other L1 | chr16: 70705336-70705969 | + | 90 | 634 | MTSS1L | Proximal | 0 | Not documented | 94 |
| | Uniquely | | | | | | | | | | | | | | |
| L1M5_dup55243 | Multi/ | No | — | — | LINE | Other L1 | chr16: 71481567-71481824 | − | 140 | 258 | ZNF23 | Proximal | 0 | Not documented | 95 |
| | Uniquely | | | | | | | | | | | | | | |
| MLT1L_dup10420 | Multi | No | — | — | LTR | ERVL- | chr16: 72118723-72118933 | − | 120 | 211 | TXNL4B | Proximal | 0 | Not documented | 96 |
| | | | | | | MaLR | | | | | | | | | |
| MIR3_dup78191 | Multi | No | — | — | SINE | MIR | chr16: 72119308-72119346 | − | 130 | 39 | TXNL4B | Proximal | 0 | Not documented | 97 |
| AluY_dup97670 | Multi/ | No | — | — | SINE | Alu | chr16: 72119629-72119917 | + | 33 | 289 | TXNL4B | Proximal | 0 | Not documented | 98 |
| | Uniquely | | | | | | | | | | | | | | |
| Tigger1_dup10551 | Uniquely | No | — | — | DNA | DNA | chr16: 72474595-72475877 | − | 56 | 1283 | PMFBP1 | Distal | 263819 | Not documented | 99 |
| L1ME3C_dup10719 | Uniquely | No | — | — | LINE | Other L1 | chr16: 72521013-72521792 | − | 130 | 780 | ZFHX3 | Distal | 294993 | Not documented | 2200 |
| L2c_dup124438 | Multi | No | — | — | LINE | L2 | chr16: 74482173-74482218 | − | 81 | 46 | GLG1 | Distal | 3639 | Not documented | 1 |
| AluSx_dup113523 | Multi | No | — | — | SINE | Alu | chr16: 74482408-74482667 | + | 60 | 260 | GLG1 | Distal | 3190 | Not documented | 2 |
| AluSx3_dup23812 | Uniquely | No | — | — | SINE | Alu | chr16: 74619035-74619320 | + | 57 | 286 | GLG1 | Proximal | 0 | Not documented | 3 |
| AluSz_dup79443 | Multi | No | — | — | SINE | Alu | chr16: 75044601-75044905 | − | 48 | 305 | ZNRF1 | Proximal | 0 | Not documented | 4 |
| FLAM_A_dup13614 | Multi | No | — | — | SINE | Alu | chr16: 75045306-75045429 | − | 85 | 124 | ZNRF1 | Proximal | 0 | Not documented | 5 |
| L1MC4a_dup23528 | Multi | No | — | — | LINE | Other L1 | chr16: 75205725-75206120 | + | 130 | 396 | ZFP1 | Proximal | 0 | Not documented | 6 |
| AluSx1_dup88152 | Multi | No | — | — | SINE | Alu | chr16: 75477705-75478005 | + | 70 | 301 | TMEM170A | Proximal | 0 | Not documented | 7 |
| 7SK_dup646 | Multi | No | — | — | Other | Other | chr16: 75532602-75532756 | + | 110 | 155 | CHST6 | Distal | 3321 | Not documented | 8 |
| | | | | | | Repeats | | | | | | | | | |
| AluSz_dup79499 | Multi | No | — | — | SINE | Alu | chr16: 75632970-75633279 | − | 50 | 310 | ADAT1 | Proximal | 0 | Not documented | 9 |
| L2_dup48548 | Multi/ | No | — | — | LINE | L2 | chr16: 768855-768923 | + | 140 | 69 | METRN | Proximal | 0 | Not documented | 10 |
| | Uniquely | | | | | | | | | | | | | | |
| AluSc8_dup16914 | Multi/ | No | — | — | SINE | Alu | chr16: 769518-769826 | + | 53 | 309 | METRN | Proximal | 0 | Not documented | 11 |
| | Uniquely | | | | | | | | | | | | | | |
| AluJr_dup63183 | Multi | No | — | — | SINE | Alu | chr16: 81014213-81014518 | + | 77 | 306 | CMC2 | Proximal | 0 | Not documented | 12 |
| L1MB2_dup7417 | Multi | No | — | — | LINE | Other L1 | chr16: 81031914-81032400 | + | 77 | 487 | CMC2 | Proximal | 0 | Not documented | 13 |
| L1PA16_dup12734 | Multi/ | No | — | — | LINE | L1PA\|B\|x | chr16: 81034125-81034957 | + | 52 | 833 | CMC2 | Proximal | 0 | Not documented | 14 |
| | Uniquely | | | | | | | | | | | | | | |
| L2c_dup121870 | Multi/ | No | — | — | LINE | L2 | chr16: 8877067-8877400 | − | 150 | 334 | TMEM186 | Proximal | 0 | Not documented | 15 |
| | Uniquely | | | | | | | | | | | | | | |
| AluYf4_dup1144 | Uniquely | No | — | — | SINE | Alu | chr16: 88869455-88869750 | − | 20 | 296 | CDT1 | Proximal | 0 | Not documented | 16 |
| MER4A_dup924 | Multi | No | — | — | LTR | ERV1 | chr16: 88880089-88880575 | + | 79 | 487 | GALNS | Proximal | 0 | Not documented | 17 |
| MIRc_dup87367 | Multi | No | — | — | SINE | MIR | chr16: 8889308-8889492 | − | 140 | 185 | TMEM186 | Proximal | 0 | Not documented | 18 |
| HERVH-int_dup5288 | Uniquely | No | — | — | LTR | ERV1 | chr16: 8932258-8935475 | − | 30 | 3218 | PMM2 | Proximal | 0 | EVE annotated | 19 |
| MIR3_dup76390 | Multi | No | — | — | SINE | MIR | chr16: 8946836-8946992 | + | 110 | 157 | CARHSP1 | Proximal | 0 | Not documented | 20 |
| AluSx_dup108995 | Multi/ | No | — | — | SINE | Alu | chr16: 8947661-8947940 | + | 55 | 280 | CARHSP1 | Proximal | 0 | Not documented | 21 |
| | Uniquely | | | | | | | | | | | | | | |
| L2a_dup148200 | Multi | No | — | — | LINE | L2 | chr16: 90025592-90025635 | − | 130 | 44 | DEF8 | Proximal | 0 | Not documented | 22 |
| L1ME4a_dup38026 | Multi | No | — | — | LINE | Other L1 | chr16: 90111213-90111404 | − | 130 | 192 | GAS8 | Proximal | 0 | Not documented | 23 |
| MIR3_dup78761 | Multi | No | — | — | SINE | MIR | chr17: 1297381-1297598 | + | 160 | 218 | YWHAE | Proximal | 0 | Not documented | 24 |
| MLT1L_dup10578 | Uniquely | No | — | — | LTR | ERVL- | chr17: 13937439-13937720 | + | 130 | 282 | COX10 | Distal | 35094 | Not documented | 2225 |
| | | | | | | MaLR | | | | | | | | | |
| L3_dup40194 | Multi | No | — | — | LINE | CR1 | chr17: 1399432-1399518 | + | 120 | 87 | INPP5K | Proximal | 0 | Not documented | 26 |
| MSTA_dup17575 | Multi | No | — | — | LTR | ERVL- | chr17: 15622049-15622446 | − | 72 | 398 | ZNF286A | Proximal | | Not documented | 27 |
| | | | | | | MaLR | | | | | | | | | |
| MER5B_dup21968 | Multi | No | — | — | DNA | DNA | chr17: 15931561-15931686 | + | 160 | 126 | TTC19 | Proximal | 0 | Not documented | 28 |
| MIRc_dup91056 | Uniquely | No | — | — | SINE | MIR | chr17: 19208000-19208124 | − | 130 | 125 | EPN2 | Proximal | 0 | Not documented | 29 |
| L1M4c_dup5473 | Multi | No | — | — | LINE | Other L1 | chr17: 19350190-19350457 | − | 110 | 268 | RNF112 | Distal | 29602 | Not documented | 30 |
| AluJb_dup119378 | Multi/ | No | — | — | SINE | Alu | chr17: 19579685-19579981 | + | 72 | 297 | ALDH3A2 | Proximal | 0 | Not documented | 31 |
| | Uniquely | | | | | | | | | | | | | | |
| MER91A_dup3160 | Uniquely | No | — | — | DNA | DNA | chr17: 20063158-20063303 | + | 130 | 146 | SPECC1 | Proximal | 0 | Not documented | 32 |
| MLT1J1_dup4382 | Uniquely | No | — | — | LTR | ERVL- | chr17: 20063448-20063781 | − | 110 | 334 | SPECC1 | Proximal | 0 | Not documented | 33 |
| | | | | | | MaLR | | | | | | | | | |
| AluSq_dup17974 | Uniquely | No | — | — | SINE | Alu | chr17: 20141073-20141354 | − | 66 | 282 | SPECC1 | Proximal | 0 | Not documented | 34 |
| L1MEg_dup15970 | Uniquely | No | — | — | LINE | Other L1 | chr17: 20152755-20153643 | + | 110 | 889 | SPECC1 | Proximal | 0 | Not documented | 35 |
| L1MC_dup10370 | Uniquely | No | — | — | LINE | Other L1 | chr17: 20214984-20215586 | − | 95 | 603 | SPECC1 | Proximal | 0 | Not documented | 36 |
| AluJo_dup59462 | Multi | No | — | — | SINE | Alu | chr17: 20217807-20217967 | + | 79 | 161 | SPECC1 | Proximal | 0 | Not documented | 37 |
| AluSx_dup116794 | Multi | No | — | — | SINE | Alu | chr17: 20904094-20904374 | + | 47 | 281 | USP22 | Proximal | 0 | Not documented | 38 |
| MER58B_dup5925 | Multi | No | — | — | DNA | DNA | chr17: 20904696-20904944 | + | 110 | 249 | USP22 | Proximal | 0 | Not documented | 39 |
| AluY_dup98938 | Multi | No | — | — | SINE | Alu | chr17: 2225797-2226105 | − | 26 | 309 | TSR1 | Proximal | 0 | Not documented | 40 |
| AluSx1_dup89255 | Multi | No | — | — | SINE | Alu | chr17: 2226116-2226358 | − | 62 | 243 | TSR1 | Proximal | 0 | Not documented | 41 |
| MER6B_dup603 | Uniquely | No | — | — | DNA | DNA | chr17: 2228265-2228474 | − | 110 | 210 | TSR1 | Proximal | 0 | Not documented | 42 |
| MIRb_dup198112 | Uniquely | No | — | — | SINE | MIR | chr17: 26727791-26728004 | + | 140 | 214 | SLC46A1 | Proximal | 0 | Not documented | 43 |
| AluSq2_dup45827 | Multi/ | No | — | — | SINE | Alu | chr17: 27383720-27384025 | + | 50 | 306 | PIPOX | Proximal | 0 | Not documented | 44 |
| | Uniquely | | | | | | | | | | | | | | |
| MIRb_dup198975 | Multi | No | — | — | SINE | MIR | chr17: 33173899-33174123 | − | 120 | 225 | CCT6B | Distal | 80756 | Not documented | 45 |
| AluSz6_dup38490 | Multi | No | — | — | SINE | Alu | chr17: 33478373-33478496 | − | 51 | 124 | UNC45B | Proximal | 0 | Not documented | 46 |
| AluSz6_dup38502 | Multi | No | — | — | SINE | Alu | chr17: 33901007-33901315 | + | 51 | 309 | PEX12 | Proximal | 500 | Not documented | 47 |
| MIR3_dup80088 | Multi | No | — | — | SINE | MIR | chr17: 34051626-34051790 | − | 130 | 165 | AP2B1 | Proximal | 0 | Not documented | 48 |
| AluSz6_dup38535 | Uniquely | No | — | — | SINE | Alu | chr17: 34894610-34894903 | − | 55 | 294 | PIGW | Proximal | 0 | Not documented | 49 |
| AluSx_dup115155 | Multi | No | — | — | SINE | Alu | chr17: 3570356-3570655 | + | 30 | 300 | TAX1BP3 | Proximal | 0 | Not documented | 2250 |
| LTR79_dup3732 | Uniquely | No | — | — | LTR | ERVL | chr17: 37529409-37529812 | + | 130 | 404 | FBXL20 | Proximal | 0 | Not documented | 51 |
| L2b_dup85305 | Multi/ | No | — | — | LINE | L2 | chr17: 38249792-38250116 | + | 120 | 325 | NR1D1 | Proximal | 0 | Not documented | 52 |
| | Uniquely | | | | | | | | | | | | | | |
| AluSx_dup118200 | Uniquely | No | — | — | SINE | Alu | chr17: 38436440-38436734 | + | 48 | 295 | WIPF2 | Proximal | 0 | Not documented | 53 |
| AluSz_dup82129 | Multi | No | — | — | SINE | Alu | chr17: 39958530-39958804 | − | 28 | 275 | LEPREL4 | Proximal | 0 | Not documented | 54 |
| AluSx1_dup91574 | Uniquely | No | — | — | SINE | Alu | chr17: 39958807-39959103 | − | 46 | 297 | LEPREL4 | Proximal | 0 | Not documented | 55 |
| MIR3_dup80401 | Multi | No | — | — | SINE | MIR | chr17: 40176904-40177016 | + | 110 | 113 | NKIRAS2 | Proximal | 0 | Not documented | 56 |
| AluJr_dup64957 | Multi | No | — | — | SINE | Alu | chr17: 40555092-40555246 | − | 76 | 155 | PTRF | Proximal | 0 | Not documented | 57 |
| AluSx_dup118425 | Multi/ | No | — | — | SINE | Alu | chr17: 40555640-40555958 | − | 55 | 319 | PTRF | Proximal | 0 | Not documented | 58 |
| | Uniquely | | | | | | | | | | | | | | |
| MER21A_dup1635 | Multi | Nc | — | — | LTR | ERVL | chr17: 40813111-40813265 | + | 130 | 155 | TUBG2 | Proximal | 0 | Not documented | 59 |
| LTR7C_dup265 | Uniquely | No | — | — | LTR | ERV1 | chr17: 41300345-41300751 | − | 57 | 407 | NBR1 | Distal | 21748 | Not documented | 60 |
| Harlequin- | Uniquely | No | — | — | LTR | ERV1 | chr17: 41318173-41319262 | − | 38 | 1090 | NBR1 | Distal | 3237 | Not documented | 61 |
| int_dup435 | | | | | | | | | | | | | | | |
| MIR3_dup80476 | Multi | No | — | — | SINE | MIR | chr17: 41844054-41844103 | − | 93 | 50 | DUSP3 | Proximal | 0 | Not documented | 62 |
| MIRc_dup91991 | Multi | No | — | — | SINE | MIR | chr17: 41844841-41845059 | + | 140 | 219 | DUSP3 | Proximal | 0 | Not documented | 63 |
| L1MC4_dup25596 | Multi | No | — | — | LINE | Other L1 | chr17: 42112932-42113098 | + | 170 | 167 | LSM12 | Proximal | 0 | Not documented | 64 |
| AluJb_dup121471 | Uniquely | No | — | — | SINE | Alu | chr17: 42253441-42253751 | + | 61 | 311 | ASB16 | Proximal | 0 | Not documented | 65 |
| MIR3_dup80508 | Uniquely | No | — | — | SINE | MIR | chr17: 42253789-42253981 | + | 120 | 193 | ASB16 | Proximal | 0 | Not documented | 66 |
| L2a_dup150639 | Uniquely | No | — | — | LINE | L2 | chr17: 42965795-42967021 | − | 100 | 1227 | EFTUD2 | Proximal | 0 | Not documented | 67 |
| MIR_dup154760 | Multi/ | No | — | — | SINE | MIR | chr17: 42983296-42983361 | + | 130 | 66 | GFAP | Proximal | 0 | Not documented | 68 |
| | Uniquely | | | | | | | | | | | | | | |
| FLAM_A_dup14067 | Multi/ | No | — | — | SINE | Alu | chr17: 42984079-42984213 | + | 100 | 135 | GFAP | Proximal | 0 | Not documented | 69 |
| | Uniquely | | | | | | | | | | | | | | |
| AluSx_dup118843 | Multi/ | No | — | — | SINE | Alu | chr17: 42989479-42989790 | − | 50 | 312 | GFAP | Proximal | 0 | Not documented | 70 |
| | Uniquely | | | | | | | | | | | | | | |
| MIR_dup154761 | Uniquely | No | — | — | SINE | MIR | chr17: 42990231-42990406 | + | 110 | 176 | GFAP | Proximal | 0 | Not documented | 71 |
| L2c_dup127161 | Multi/ | No | — | — | LINE | L2 | chr17: 43100732-43101146 | − | 160 | 415 | DCAKD | Proximal | 0 | Not documented | 72 |
| | Uniquely | | | | | | | | | | | | | | |
| AluJr_dup65152 | Multi/ | No | — | — | SINE | Alu | chr17: 43189108-43189423 | + | 55 | 316 | PLCD3 | Proximal | 0 | Not documented | 73 |
| | Uniquely | | | | | | | | | | | | | | |
| AluJr4_dup14906 | Uniquely | No | — | — | SINE | Alu | chr17: 44040010-44040318 | + | 62 | 309 | MAPT | Proximal | 0 | Not documented | 74 |
| LTR33B_dup1414 | Multi | No | — | — | LTR | ERVL | chr17: 48562604-48562862 | − | 130 | 259 | RSAD1 | Proximal | 0 | Not documented | 2275 |
| LTR33B_dup1415 | Multi | No | — | — | LTR | ERVL | chr17: 48562893-48563000 | − | 130 | 108 | RSAD1 | Proximal | 0 | Not documented | 76 |
| MIR3_dup80878 | Multi/ | No | — | — | SINE | MIR | chr17: 48768721-48768843 | − | 84 | 123 | ABCC3 | Proximal | 0 | Not documented | 77 |
| | Uniquely | | | | | | | | | | | | | | |
| AluSz_dup82869 | Multi | No | — | — | SINE | Alu | chr17: 48771210-48771510 | + | 50 | 301 | ANKRD40 | Proximal | 0 | Not documented | 78 |
| AluJr_dup65430 | Multi | No | — | — | SINE | Alu | chr17: 48772448-48772566 | + | 54 | 119 | ANKRD40 | Proximal | 0 | Not documented | 79 |
| MIRb_dup196690 | Multi | No | — | — | SINE | MIR | chr17: 5016546-5016763 | − | 140 | 218 | ZNF232 | Proximal | 0 | Not documented | 80 |
| MLT1A_dup7944 | Uniquely | No | — | — | LTR | ERVL- | chr17: 5117183-5117559 | + | 94 | 377 | SCIMP | Proximal | 0 | Not documented | 81 |
| | | | | | | MaLR | | | | | | | | | |
| L1MC4_dup25223 | Uniquely | No | — | — | LINE | Other L1 | chr17: 5381046-5381375 | + | 90 | 330 | DERL2 | Proximal | 0 | Not documented | 82 |
| AluJr4_dup14971 | Multi | No | — | — | SINE | Alu | chr17: 5393186-5393397 | − | 79 | 212 | MIS12 | Proximal | 0 | Not documented | 83 |
| AluSx_dup119791 | Uniquely | No | — | — | SINE | Alu | chr17: 55640177-55640482 | + | 45 | 306 | MSI2 | Proximal | 0 | Not documented | 84 |
| L2c_dup128112 | Multi | No | — | — | LINE | L2 | chr17: 57187416-57187526 | − | 110 | 111 | SKA2 | Proximal | 0 | Not documented | 85 |
| AluSg7_dup7058 | Multi | No | — | — | SINE | Alu | chr17: 57187872-57188172 | + | 35 | 301 | SKA2 | Proximal | 0 | Not documented | 86 |
| L1ME4a_dup39178 | Multi | No | — | — | LINE | Other L1 | chr17: 57188598-57188800 | − | 150 | 203 | SKA2 | Proximal | 0 | Not documented | 87 |
| AluSq2_dup46846 | Multi | No | — | — | SINE | Alu | chr17: 57916349-57916651 | − | 40 | 303 | VMP1 | Proximal | 0 | Not documented | 88 |
| L4_dup15385 | Multi | No | — | — | LINE | RTE | chr17: 58179944-58180005 | − | 97 | 62 | HEATR6 | Distal | 23653 | Not documented | 89 |
| MLT1K_dup15635 | Multi | No | — | — | LTR | ERVL- | chr17: 6027352-6027780 | + | 140 | 429 | WSCD1 | Proximal | 0 | Not documented | 90 |
| | | | | | | MaLR | | | | | | | | | |
| FLAM_A_dup14228 | Multi | No | — | — | SINE | Alu | chr17: 60526161-60526293 | + | 72 | 133 | METTL2A | Proximal | 0 | Not documented | 91 |
| MIRc_dup92933 | Multi | No | — | — | SINE | MIR | chr17: 61905187-61905408 | − | 140 | 222 | PSMC5 | Proximal | 0 | Not documented | 92 |
| L1ME4a_dup39320 | Uniquely | No | — | — | LINE | Other L1 | chr17: 63012954-63013287 | − | 140 | 334 | GNA13 | Proximal | 0 | Not documented | 93 |
| AluSq_dup18487 | Multi | No | — | — | SINE | Alu | chr17: 64804822-64805126 | − | 44 | 305 | PRKCA | Proximal | 0 | Not documented | 94 |
| L1PREC2_dup7058 | Uniquely | No | — | — | LINE | L1PA\|B\|x | chr17: 65491491-65491863 | − | 40 | 373 | PITPNC1 | Proximal | 0 | Not documented | 95 |
| AluY_dup103242 | Multi/ | No | — | — | SINE | Alu | chr17: 66038717-66038998 | + | 23 | 282 | KPNA2 | Proximal | 0 | Not documented | 96 |
| | Uniquely | | | | | | | | | | | | | | |
| AluJb_dup123723 | Multi | No | — | — | SINE | Alu | chr17: 66122123-66122426 | − | 69 | 304 | KPNA2 | Distal | 79166 | Not documented | 97 |
| L1ME4a_dup38045 | Multi | No | — | — | LINE | Other L1 | chr17: 674677-674826 | + | 160 | 150 | GLOD4 | Proximal | 0 | Not documented | 98 |
| FLAM_C_dup18328 | Uniquely | No | — | — | SINE | Alu | chr17: 6905826-6905957 | + | 79 | 132 | ALOX12 | Proximal | 0 | Not documented | 99 |
| L3_dup41281 | Multi | No | — | — | LINE | CR1 | chr17: 70067150-70067250 | + | 150 | 101 | AC007461.1 | Distal | 30279 | Not documented | 2300 |
| LTR33_dup8325 | Multi | No | — | — | LTR | ERVL | chr17: 70077995-70078138 | − | 150 | 144 | SOX9 | Distal | 39024 | Not documented | 1 |
| MLT1L_dup10811 | Multi | No | — | — | LTR | ERVL- | chr17: 70081165-70081293 | − | 110 | 129 | SOX9 | Distal | 35869 | Not documented | 2 |
| | | | | | | MaLR | | | | | | | | | |
| X8_LINE_dup280 | Multi/ | No | — | — | LINE | CR1 | chr17: 70121870-70121938 | − | 150 | 69 | SOX9 | Proximal | 0 | Not documented | 3 |
| | Uniquely | | | | | | | | | | | | | | |
| AluJb_dup123904 | Multi | No | — | — | SINE | Alu | chr17: 70400523-70400818 | − | 65 | 296 | SLC39A11 | Distal | 241271 | Not documented | 4 |
| MIRb_dup202390 | Uniquely | No | — | — | SINE | MIR | chr17: 70424010-70424197 | + | 130 | 188 | SLC39A11 | Distal | 217892 | Not documented | 5 |
| AluSz_dup84029 | Multi/ | No | — | — | SINE | Alu | chr17: 70474465-70474773 | + | 76 | 309 | SLC39A11 | Distal | 167316 | Not documented | 6 |
| | Uniquely | | | | | | | | | | | | | | |
| MIRb_dup202396 | Uniquely | No | — | — | SINE | MIR | chr17: 70499760-70499991 | − | 140 | 232 | SLC39A11 | Distal | 142098 | Not documented | 7 |
| L1ME2z_dup6778 | Multi | No | — | — | LINE | Other L1 | chr17: 70501817-70502142 | + | 91 | 326 | SLC39A11 | Distal | 139947 | Not documented | 8 |
| AluSp_dup42385 | Multi/ | No | — | — | SINE | Alu | chr17: 70502143-70502444 | + | 45 | 302 | SLC39A11 | Distal | 139645 | Not documented | 9 |
| | Uniquely | | | | | | | | | | | | | | |
| AluY_dup103410 | Multi/ | No | — | — | SINE | Alu | chr17: 70502445-70502741 | + | 34 | 297 | SLC39A11 | Distal | 139348 | Not documented | 10 |
| | Uniquely | | | | | | | | | | | | | | |
| L1MB3_dup15007 | Multi | No | — | — | LINE | Other L1 | chr17: 70520522-70520846 | + | 73 | 325 | SLC39A11 | Distal | 121243 | Not documented | 11 |
| FLAM_C_dup19156 | Multi | No | — | — | SINE | Alu | chr17: 70520847-70520981 | + | 71 | 135 | SLC39A11 | Distal | 121108 | Not documented | 12 |
| MER53_dup5173 | Multi/ | No | — | — | DNA | DNA | chr17: 71280191-71280358 | + | 92 | 168 | CDC42EP4 | Proximal | 0 | Not documented | 13 |
| | Uniquely | | | | | | | | | | | | | | |
| L2c_dup125388 | Multi | No | — | — | LINE | L2 | chr17: 7217060-7217163 | + | 160 | 104 | GPS2 | Proximal | 0 | Not documented | 14 |
| MIRc_dup93300 | Multi | No | — | — | SINE | MIR | chr17: 72766776-72766836 | + | 100 | 61 | NAT9 | Proximal | 0 | Not documented | 15 |
| AluSz6_dup39678 | Uniquely | No | — | — | SINE | Alu | chr17: 73425303-73425614 | + | 45 | 312 | KIAA0195 | Distal | 11627 | Not documented | 16 |
| AluSp_dup40337 | Multi | No | — | — | SINE | Alu | chr17: 7360355-7360648 | + | 30 | 294 | CHRNB1 | Proximal | 0 | Not documented | 17 |
| L2a_dup152452 | Multi/ | No | — | — | LINE | L2 | chr17: 73870242-73870307 | − | 90 | 66 | TRIM47 | Proximal | 0 | Not documented | 18 |
| | Uniquely | | | | | | | | | | | | | | |
| L3b_dup6271 | Multi | No | — | — | LINE | CR1 | chr17: 7404245-7404309 | − | 88 | 65 | POLRZA | Proximal | 0 | Not documented | 19 |
| MER112_dup3993 | Multi | No | — | — | DNA | DNA | chr17: 74559565-74559613 | − | 120 | 49 | ST6GALNAC2 | Proximal | 180 | Not documented | 20 |
| AluSx_dup121656 | Multi | No | — | — | SINE | Alu | chr17: 74559800-74560140 | − | 61 | 341 | ST6GALNAC2 | Proximal | 0 | Not documented | 21 |
| AluSq_dup18605 | Uniquely | No | — | — | SINE | Alu | chr17: 75090117-75090398 | − | 37 | 282 | SEC14L1 | Proximal | 0 | Not documented | 22 |
| AluSp_dup40355 | Multi/ | No | — | — | SINE | Alu | chr17: 7559973-7560298 | − | 35 | 326 | ATP1B2 | Proximal | 0 | Not documented | 23 |
| | Uniquely | | | | | | | | | | | | | | |
| AluJb_dup118675 | Multi | No | — | — | SINE | Alu | chr17: 7571867-7572174 | + | 72 | 308 | TP53 | Proximal | 0 | Not documented | 24 |
| MIRb_dup196902 | Multi/ | No | — | — | SINE | MIR | chr17: 7572460-7572562 | − | 130 | 103 | TP53 | Proximal | 0 | Not documented | 2325 |
| | Uniquely | | | | | | | | | | | | | | |
| L1MA4_dup9180 | Uniquely | No | — | — | LINE | Other L1 | chr17: 75974480-75975490 | + | 100 | 1011 | TNRC6C | Distal | 24760 | Not documented | 26 |
| AluJb_dup124520 | Uniquely | No | — | — | SINE | Alu | chr17: 76374101-76374411 | + | 70 | 311 | PGS1 | Proximal | 311 | Not documented | 27 |
| AluSx_dup122212 | Multi | No | — | — | SINE | Alu | chr17: 79640296-79640423 | + | 35 | 128 | CCDC137 | Proximal | 0 | Not documented | 28 |
| LTR52_dup989 | Multi | No | — | — | LTR | ERVL | chr17: 79640548-79640704 | − | 110 | 157 | CCDC137 | Proximal | 0 | Not documented | 29 |
| AluSp_dup42658 | Multi | No | — | — | SINE | Alu | chr17: 79648221-79648530 | − | 50 | 310 | ARL16 | Proximal | 0 | Not documented | 30 |
| AluJb_dup124834 | Multi | No | — | — | SINE | Alu | chr17: 79851398-79851455 | − | 72 | 58 | ANAPC11 | Proximal | 0 | Not documented | 31 |
| MLT2B4_dup4142 | Multi | No | — | — | LTR | ERVL | chr17: 79890133-79890347 | + | 88 | 215 | PYCR1 | Proximal | 0 | Not documented | 32 |
| MER31-int_dup1853 | Multi | No | — | — | LTR | ERV1 | chr17: 81188246-81188689 | − | 99 | 444 | METRNL | Distal | 135383 | Not documented | 33 |
| MIR_dup152645 | Uniquely | No | — | — | SINE | MIR | chr17: 8279553-8279746 | + | 120 | 194 | KRBA2 | Proximal | 0 | Not documented | 34 |
| AluJb_dup125513 | Multi | No | — | — | SINE | Alu | chr18: 10551101-10551233 | − | 89 | 133 | NAPG | Proximal | 0 | Not documented | 35 |
| MER57B2_dup992 | Multi/ | No | — | — | LTR | ERV1 | chr18: 19209358-19209738 | − | 69 | 381 | SNRPD1 | Proximal | 0 | Not documented | 36 |
| | Uniquely | | | | | | | | | | | | | | |
| AluSx1_dup95327 | Multi | No | — | — | SINE | Alu | chr18: 23596405-23596717 | + | 33 | 313 | SS18 | Proximal | 0 | Not documented | 37 |
| L1ME1_dup28876 | Multi | No | — | — | LINE | Other L1 | chr18: 23606205-23607330 | + | 100 | 1126 | SS18 | Proximal | 0 | Not documented | 38 |
| AluSc8_dup19087 | Multi | No | — | — | SINE | Alu | chr18: 23607331-23607666 | + | 42 | 336 | SS18 | Proximal | 0 | Not documented | 39 |
| L2b_dup87398 | Multi/ | No | — | — | LINE | L2 | chr18: 24433983-24434064 | − | 130 | 82 | AQP4 | Proximal | 0 | Not documented | 40 |
| | Uniquely | | | | | | | | | | | | | | |
| Charlie1a_dup5306 | Uniquely | No | — | — | DNA | DNA | chr18: 24476496-24477665 | + | 86 | 1170 | CHST9 | Distal | 17931 | Not documented | 41 |
| MER102b_dup3653 | Uniquely | No | — | — | DNA | DNA | chr18: 2538394-2538727 | − | 140 | 334 | METTL4 | Proximal | 0 | Not documented | 42 |
| AluJb_dup126238 | Multi | No | — | — | SINE | Alu | chr18: 25708381-25708664 | + | 70 | 284 | CDH2 | Proximal | 0 | Not documented | 43 |
| L1PA8_dup7494 | Multi/ | No | — | — | LINE | L1PA\|B\|x | chr18: 25723750-25724140 | + | 36 | 391 | CDH2 | Proximal | 0 | Not documented | 44 |
| | Uniquely | | | | | | | | | | | | | | |
| MIRb_dup204764 | Uniquely | No | — | — | SINE | MIR | chr18: 29907711-29907946 | + | 120 | 236 | GAREM | Proximal | 0 | Not documented | 45 |
| MIRb_dup204765 | Uniquely | No | — | — | SINE | MIR | chr18: 29915943-29916111 | + | 160 | 169 | GAREM | Proximal | 0 | Not documented | 46 |
| AluY_dup105497 | Multi | No | — | — | SINE | Alu | chr18: 32083244-32083547 | − | 36 | 304 | DTNA | Proximal | 0 | Not documented | 47 |
| L1MD1_dup6225 | Multi | No | — | — | LINE | Other L1 | chr18: 32083548-32083790 | − | 98 | 243 | DTNA | Proximal | 0 | Not documented | 48 |
| AluY_dup105498 | Multi/ | No | — | — | SINE | Alu | chr18: 32083791-32084119 | − | 35 | 329 | DTNA | Proximal | 0 | Not documented | 49 |
| | Uniquely | | | | | | | | | | | | | | |
| L1MD1_dup6226 | Multi/ | No | — | — | LINE | Other L1 | chr18: 32084120-32084956 | − | 98 | 837 | DTNA | Proximal | 0 | Not documented | 2350 |
| | Uniquely | | | | | | | | | | | | | | |
| MLT1D_dup18463 | Multi/ | No | — | — | LTR | ERVL- | chr18: 32084957-32085450 | − | 110 | 494 | DTNA | Proximal | 0 | Not documented | 51 |
| | Uniquely | | | | | MaLR | | | | | | | | | |
| L1MD1_dup6227 | Multi | No | — | — | LINE | Other L1 | chr18: 32085451-32086874 | − | 98 | 1424 | DTNA | Proximal | 0 | Not documented | 52 |
| MamRep605_dup4194 | Multi | No | — | — | Other | Other | chr18: 32090948-32091116 | − | 130 | 169 | DTNA | Proximal | 0 | Not documented | 53 |
| | | | | | | Repeats | | | | | | | | | |
| MIR_dup158397 | Multi | No | — | — | SINE | MIR | chr18: 32091244-32091415 | − | 150 | 172 | DTNA | Proximal | 0 | Not documented | 54 |
| AluSq2_dup48232 | Uniquely | No | — | — | SINE | Alu | chr18: 32113053-32113352 | + | 49 | 300 | DTNA | Proximal | 0 | Not documented | 55 |
| L1PA8_dup7514 | Uniquely | No | — | — | LINE | L1PA\|B\|x | chr18: 32114443-32115769 | − | 34 | 1327 | DTNA | Proximal | 0 | Not documented | 56 |
| HAL1_dup25084 | Multi/ | No | — | — | LINE | Other L1 | chr18: 32163399-32165869 | + | 130 | 2471 | DTNA | Proximal | 0 | Not documented | 57 |
| | Uniquely | | | | | | | | | | | | | | |
| Tigger1_dup11096 | Uniquely | No | — | — | DNA | DNA | chr18: 32192892-32194965 | + | 67 | 2074 | DTNA | Proximal | 0 | Not documented | 58 |
| MIRb_dup204919 | Uniquely | No | — | — | SINE | MIR | chr18: 32271317-32271440 | + | 120 | 124 | DTNA | Proximal | 0 | Not documented | 59 |
| L1PA7_dup12259 | Uniquely | No | — | — | LINE | L1PA\|B\|x | chr18: 32304745-32306224 | + | 32 | 1480 | DTNA | Proximal | 0 | Not documented | 60 |
| L1PA16_dup13114 | Multi | No | — | — | LINE | L1PA\|B\|x | chr18: 32318875-32319065 | − | 56 | 191 | DTNA | Proximal | 0 | Not documented | 61 |
| AluSq2_dup48234 | Multi | No | — | — | SINE | Alu | chr18: 32349690-32349984 | − | 45 | 295 | DTNA | Proximal | 0 | Not documented | 62 |
| MLT1A0_dup18867 | Multi | No | — | — | LTR | ERVL- | chr18: 32349985-32350141 | − | 71 | 157 | DTNA | Proximal | 0 | Not documented | 63 |
| | | | | | | MaLR | | | | | | | | | |
| AluY_dup105506 | Multi/ | No | — | — | SINE | Alu | chr18: 32364897-32365175 | − | 33 | 279 | DTNA | Proximal | 0 | Not documented | 64 |
| | Uniquely | | | | | | | | | | | | | | |
| AluY_dup105507 | Multi/ | No | — | — | SINE | Alu | chr18: 32404158-32404454 | + | 28 | 297 | DTNA | Proximal | 0 | Not documented | 65 |
| | Uniquely | | | | | | | | | | | | | | |
| L1PA8_dup7515 | Multi | No | — | — | LINE | L1PA\|B\|x | chr18: 32404467-32404544 | + | 18 | 78 | DTNA | Proximal | 0 | Not documented | 66 |
| AluSq2_dup48236 | Uniquely | No | — | — | SINE | Alu | chr18: 32411450-32411746 | + | 55 | 297 | DTNA | Proximal | 0 | Not documented | 67 |
| MSTB1_dup4589 | Multi | No | — | — | LTR | ERVL- | chr18: 32414108-32414177 | − | 68 | 70 | DTNA | Proximal | 0 | Not documented | 68 |
| | | | | | | MaLR | | | | | | | | | |
| AluSq2_dup48237 | Multi | No | — | — | SINE | Alu | chr18: 32414178-32414479 | + | 39 | 302 | DTNA | Proximal | 0 | Not documented | 69 |
| MSTB1_dup4590 | Multi/ | No | — | — | LTR | ERVL- | chr18: 32414480-32414841 | − | 68 | 362 | DTNA | Proximal | 0 | Not documented | 70 |
| | Uniquely | | | | | MaLR | | | | | | | | | |
| AluSz_dup85746 | Multi/ | No | — | — | SINE | Alu | chr18: 32416335-32416632 | + | 38 | 298 | DTNA | Proximal | 0 | Not documented | 71 |
| | Uniquely | | | | | | | | | | | | | | |
| L2b_dup87589 | Uniquely | No | — | — | LINE | L2 | chr18: 32422400-32423005 | + | 160 | 606 | DTNA | Proximal | 0 | Not documented | 72 |
| MLT1B_dup16130 | Uniquely | No | — | — | LTR | ERVL- | chr18: 32423358-32423729 | − | 76 | 372 | DTNA | Proximal | 0 | Not documented | 73 |
| | | | | | | MaLR | | | | | | | | | |
| L2a_dup154163 | Multi/ | No | — | — | LINE | L2 | chr18: 32432443-32433039 | + | 130 | 597 | DTNA | Proximal | 0 | Not documented | 74 |
| | Uniquely | | | | | | | | | | | | | | |
| MIR_dup158419 | Multi | No | — | — | SINE | MIR | chr18: 32436554-32436774 | + | 150 | 221 | DTNA | Proximal | 0 | Not documented | 2375 |
| AluY_dup105509 | Multi | No | — | — | SINE | Alu | chr18: 32444210-32444516 | − | 13 | 307 | DTNA | Proximal | 0 | Not documented | 76 |
| MamRep434_dup2018 | Multi | No | — | — | DNA | DNA | chr18: 32444525-32444668 | + | 110 | 144 | DTNA | Proximal | 0 | Not documented | 77 |
| L4_dup15665 | Multi/ | No | — | — | LINE | RTE | chr18: 32447960-32448445 | − | 140 | 486 | DTNA | Proximal | 0 | Not documented | 78 |
| | Uniquely | | | | | | | | | | | | | | |
| MER5A_dup31718 | Multi/ | No | — | — | DNA | DNA | chr18: 32469199-32469385 | + | 85 | 187 | DTNA | Proximal | 0 | Not documented | 79 |
| | Uniquely | | | | | | | | | | | | | | |
| LTR41_dup1602 | Multi | No | — | — | LTR | ERVL | chr18: 32842654-32842959 | + | 92 | 306 | ZNF397 | Proximal | 0 | Not documented | 80 |
| AluJo_dup62774 | Multi | No | — | — | SINE | Alu | chr18: 32843056-32843237 | + | 70 | 182 | ZNF397 | Proximal | 0 | Not documented | 81 |
| AluJr_dup67499 | Multi | No | — | — | SINE | Alu | chr18: 32887935-32888089 | + | 70 | 155 | ZSCAN30 | Distal | 17740 | Not documented | 82 |
| AluSx3_dup25763 | Multi | No | — | — | SINE | Alu | chr18: 32888183-32888406 | + | 40 | 224 | ZSCAN30 | Distal | 17988 | Not documented | 83 |
| MLT1K_dup16215 | Multi/ | No | — | — | LTR | ERVL- | chr18: 32912307-32912778 | − | 120 | 472 | ZNF24 | Proximal | 0 | Not documented | 84 |
| | Uniquely | | | | | MaLR | | | | | | | | | |
| L1M5_dup58270 | Multi | No | — | — | LINE | Other L1 | chr18: 32912781-32913164 | + | 120 | 384 | ZNF24 | Proximal | 0 | Not documented | 85 |
| Tigger3c_dup2148 | Uniquely | No | — | — | DNA | DNA | chr18: 33253787-33254344 | − | 63 | 558 | GALNT1 | Proximal | 0 | Not documented | 86 |
| MIRc_dup94378 | Multi | No | — | — | SINE | MIR | chr18: 33559118-33559187 | + | 80 | 70 | C18orf21 | Proximal | 0 | Not documented | 87 |
| MER3_dup9776 | Multi | No | — | — | DNA | DNA | chr18: 34376226-34376412 | + | 83 | 187 | TPGS2 | Proximal | 0 | Not documented | 88 |
| L3_dup41870 | Multi | No | — | — | LINE | CR1 | chr18: 34387717-34387820 | − | 140 | 104 | TPGS2 | Proximal | 0 | Not documented | 89 |
| MER68B_dup397 | Uniquely | No | — | — | LTR | ERVL | chr18: 39621887-39622421 | + | 97 | 535 | PIK3C3 | Proximal | 0 | Not documented | 90 |
| AluSp_dup43247 | Multi | No | — | — | SINE | Alu | chr18: 43835404-43835713 | − | 42 | 310 | C18orf25 | Proximal | 0 | Not documented | 91 |
| AluJo_dup62929 | Multi | No | — | — | SINE | Alu | chr18: 44402920-44403203 | + | 96 | 284 | PIAS2 | Proximal | 0 | Not documented | 92 |
| L2_dup51990 | Uniquely | No | — | — | LINE | L2 | chr18: 44525757-44526123 | − | 160 | 367 | KATNAL2 | Proximal | 0 | Not documented | 93 |
| MER123_dup31 | Multi | No | — | — | DNA | DNA | chr18: 44634246-44634352 | − | 110 | 107 | HDHD2 | Proximal | 0 | Not documented | 94 |
| L2b_dup88109 | Multi | No | — | — | LINE | L2 | chr18: 47795434-47795520 | + | 140 | 87 | MBD1 | Proximal | 0 | Not documented | 95 |
| MLT2F_dup1775 | Multi | No | — | — | LTR | ERVL | chr18: 47795693-47796348 | + | 68 | 656 | MBD1 | Proximal | 0 | Not documented | 96 |
| MIR3_dup83415 | Multi/ | No | — | — | SINE | MIR | chr18: 48256557-48256734 | − | 140 | 178 | MAPK4 | Proximal | 0 | Not documented | 97 |
| | Uniquely | | | | | | | | | | | | | | |
| L2a_dup155040 | Uniquely | No | — | — | LINE | L2 | chr18: 48553155-48553406 | − | 100 | 252 | RP11-729L2.2 | Proximal | 0 | Not documented | 98 |
| AluSg_dup36194 | Uniquely | No | — | — | SINE | Alu | chr18: 48589222-48589521 | − | 27 | 300 | SMAD4 | Proximal | 0 | Not documented | 99 |
| L2a_dup155043 | Uniquely | No | — | — | LINE | L2 | chr18: 48590560-48590911 | + | 150 | 352 | SMAD4 | Proximal | 0 | Not documented | 2400 |
| L2c_dup129372 | Multi/ | No | — | — | LINE | L2 | chr18: 5239079-5239161 | − | 150 | 83 | C18orf42 | Distal | 41578 | Not documented | 1 |
| | Uniquely | | | | | | | | | | | | | | |
| MLT2B2_dup2031 | Uniquely | No | — | — | LTR | ERVL | chr18: 5244238-5244673 | − | 75 | 436 | ZBTB14 | Distal | 44346 | Not documented | 2 |
| AluJr_dup67882 | Multi | No | — | — | SINE | Alu | chr18: 52897176-52897399 | + | 73 | 224 | TCF4 | Proximal | 0 | Not documented | 3 |
| AluSq2_dup48506 | Uniquely | No | — | — | SINE | Alu | chr18: 54269248-54269541 | − | 46 | 294 | TXNL1 | Proximal | 0 | Not documented | 4 |
| MIRc_dup95510 | Multi | No | — | — | SINE | MIR | chr18: 56653423-56653646 | − | 130 | 224 | ZNF532 | Proximal | 0 | Not documented | 5 |
| L1MA4_dup9379 | Multi | No | — | — | LINE | Other L1 | chr18: 56834904-56837217 | + | 95 | 2314 | SEC11C | Distal | 8837 | Not documented | 6 |
| ERV3-16A3_I- | Multi | No | — | — | LTR | ERVL | chr18: 58211323-58212239 | − | 130 | 917 | MC4R | Distal | 171323 | Not documented | 7 |
| int_dup5812 | | | | | | | | | | | | | | | |
| MLT1G1_dup3257 | Uniquely | No | — | — | LTR | ERVL- | chr18: 61048498-61048892 | + | 120 | 395 | VPS4B | Distal | 7532 | Not documented | 8 |
| | | | | | | MaLR | | | | | | | | | |
| AluSz6_dup40693 | Multi | No | — | — | SINE | Alu | chr18: 61057277-61057344 | − | 54 | 68 | VPS4B | Proximal | 0 | Not documented | 9 |
| AluSx1_dup96312 | Multi | No | — | — | SINE | Alu | chr18: 61057345-61057606 | − | 50 | 262 | VPS4B | Proximal | 0 | Not documented | 10 |
| L1PA16_dup13268 | Uniquely | No | — | — | LINE | L1PA\|B\|x | chr18: 65130676-65132717 | − | 75 | 2042 | DSEL | Distal | 41103 | Not documented | 11 |
| AluSg_dup36364 | Multi | No | — | — | SINE | Alu | chr18: 65174305-65174612 | + | 58 | 308 | DSEL | Proximal | 0 | Not documented | 12 |
| Helitron2Na_{—} | Multi | No | — | — | Other | Other | chr18: 65945724-65945861 | + | 140 | 138 | TMX3 | Distal | 395065 | Not documented | 13 |
| Mam_dup428 | | | | | | Repeats | | | | | | | | | |
| Charlie15a_{—} | Multi | No | — | — | DNA | DNA | chr18: 66343308-66343428 | − | 130 | 121 | TMX3 | Proximal | 0 | Not documented | 14 |
| dup3228 | | | | | | | | | | | | | | | |
| L1PB2_dup2660 | Multi | No | — | — | LINE | L1PA\|B\|x | chr18: 72260707-72261085 | − | 42 | 379 | ZNF407 | Distal | 4022 | Not documented | 15 |
| AluY_dup104234 | Multi | No | — | — | SINE | Alu | chr18: 722811-723111 | + | 23 | 301 | YES1 | Proximal | 0 | Not documented | 16 |
| AluY_dup104236 | Multi | No | — | — | SINE | Alu | chr18: 733502-733810 | + | 28 | 309 | YES1 | Proximal | 0 | Not documented | 17 |
| L1PA16_dup13328 | Uniquely | No | — | — | LINE | L1PA\|B\|x | chr18: 75151387-75152208 | − | 59 | 822 | GALR1 | Distal | 170530 | Not documented | 18 |
| AluJo_dup63488 | Uniquely | No | — | — | SINE | Alu | chr18: 77438792-77438990 | − | 66 | 199 | CTDP1 | Proximal | 812 | Not documented | 19 |
| MER68_dup1312 | Uniquely | No | — | — | LTR | ERVL | chr18: 77909190-77909554 | − | 140 | 365 | AC139100.2 | Proximal | 0 | Not documented | 20 |
| MIR3_dup82159 | Multi | No | — | — | SINE | MIR | chr18: 9956099-9956259 | − | 140 | 161 | VAPA | Proximal | 0 | Not documented | 21 |
| AluJo_dup64483 | Multi | No | — | — | SINE | Alu | chr19: 10664244-10664515 | − | 60 | 272 | KRI1 | Proximal | 0 | Not documented | 22 |
| AluJr_dup69340 | Multi | No | — | — | SINE | Alu | chr19: 11040757-11040876 | + | 95 | 120 | C19orf52 | Proximal | 0 | Not documented | 23 |
| AluJo_dup64564 | Multi | No | — | — | SINE | Alu | chr19: 11276241-11276529 | − | 58 | 289 | KANK2 | Proximal | 0 | Not documented | 24 |
| MIRc_dup96336 | Multi | No | — | — | SINE | MIR | chr19: 11467519-11467603 | + | 110 | 85 | DKFZP761J1410 | Proximal | 0 | Not documented | 2425 |
| L1MA4A_dup5926 | Uniquely | No | — | — | LINE | Other L1 | chr19: 11893645-11894870 | + | 69 | 1226 | ZNF441 | Proximal | 0 | Not documented | 26 |
| AluYg6_dup724 | Multi | No | — | — | SINE | Alu | chr19: 12308319-12308450 | − | 20 | 132 | ZNF136 | Distal | 8256 | Not documented | 27 |
| L1MA1_dup4053 | Multi | No | — | — | LINE | Other L1 | chr19: 12308451-12309666 | − | 36 | 1216 | ZNF136 | Distal | 8388 | Not documented | 28 |
| X6B_LINE_dup420 | Multi | No | — | — | LINE | CR1 | chr19: 1270918-1270988 | + | 140 | 71 | CIRBP | Proximal | 0 | Not documented | 29 |
| AluY_dup108569 | Multi | No | — | — | SINE | Alu | chr19: 12740415-12740697 | + | 32 | 283 | ZNF791 | Proximal | 0 | Not documented | 30 |
| AluJr_dup69499 | Multi | No | — | — | SINE | Alu | chr19: 12740708-12740987 | + | 85 | 280 | ZNF791 | Proximal | 0 | Not documented | 31 |
| AluSx_dup127539 | Multi | No | — | — | SINE | Alu | chr19: 12884907-12885219 | − | 61 | 313 | HOOK2 | Proximal | 0 | Not documented | 32 |
| L2a_dup157339 | Multi | No | — | — | LINE | L2 | chr19: 12885220-12885365 | + | 130 | 146 | HOOK2 | Proximal | 0 | Not documented | 33 |
| MIRb_dup208350 | Multi/ | No | — | — | SINE | MIR | chr19: 12911536-12911731 | − | 140 | 196 | PRDX2 | Proximal | 0 | Not documented | 34 |
| | Uniquely | | | | | | | | | | | | | | |
| L2_dup52885 | Multi | No | — | — | LINE | L2 | chr19: 14602400-14602639 | + | 150 | 240 | GIPC1 | Proximal | 0 | Not documented | 35 |
| MLT1F_dup3878 | Multi | No | — | — | LTR | ERVL- | chr19: 1597355-1597871 | + | 100 | 517 | UQCR11 | Proximal | 0 | Not documented | 36 |
| | | | | | | MaLR | | | | | | | | | |
| AluSz_dup88509 | Multi | No | — | — | SINE | Alu | chr19: 16212879-16213167 | + | 52 | 289 | TPM4 | Proximal | 0 | Not documented | 37 |
| MER117_dup4197 | Multi/ | No | — | — | DNA | DNA | chr19: 16302429-16302574 | − | 150 | 146 | FAM32A | Proximal | 0 | Not documented | 38 |
| | Uniquely | | | | | | | | | | | | | | |
| AluSx_dup128197 | Multi/ | No | — | — | SINE | Alu | chr19: 16741914-16742229 | + | 56 | 316 | SMIM7 | Proximal | 0 | Not documented | 39 |
| | Uniquely | | | | | | | | | | | | | | |
| MamGypLTR3_dup888 | Multi/ | No | — | — | LTR | Gypsy | chr19: 16742298-16742518 | + | 140 | 221 | SMIM7 | Proximal | 0 | Not documented | 40 |
| | Uniquely | | | | | | | | | | | | | | |
| L2c_dup132761 | Multi | No | — | — | LINE | L2 | chr19: 17330168-17330372 | − | 150 | 205 | USE1 | Proximal | 0 | Not documented | 41 |
| L2c_dup132762 | Multi | No | — | — | LINE | L2 | chr19: 17339782-17339947 | + | 150 | 166 | OCEL1 | Proximal | 0 | Not documented | 42 |
| L1MC5_dup18967 | Multi | No | — | — | LINE | Other L1 | chr19: 17417358-17417651 | − | 130 | 294 | MRPL34 | Proximal | 0 | Not documented | 43 |
| L2b_dup89790 | Uniquely | No | — | — | LINE | L2 | chr19: 18044512-18044744 | + | 130 | 233 | CCDC124 | Proximal | 0 | Not documented | 44 |
| AluSz6_dup41580 | Multi | No | — | — | SINE | Alu | chr19: 18280737-18281039 | + | 48 | 303 | PIK3R2 | Proximal | 0 | Not documented | 45 |
| AluJr_dup70114 | Multi | No | — | — | SINE | Alu | chr19: 18307402-18307540 | + | 65 | 139 | MPV17L2 | Proximal | 0 | Not documented | 46 |
| L1M5_dup59310 | Multi/ | No | — | — | LINE | Other L1 | chr19: 1875105-1875287 | + | 150 | 183 | ABHD17A | Proximal | 1523 | Not documented | 47 |
| | Uniquely | | | | | | | | | | | | | | |
| L2_dup52956 | Multi | No | — | — | LINE | L2 | chr19: 18988588-18988795 | + | 140 | 208 | GDF1 | Proximal | 0 | Not documented | 48 |
| MIR3_dup84619 | Multi | No | — | — | SINE | MIR | chr19: 19049011-19049170 | − | 150 | 160 | HOMER3 | Proximal | 0 | Not documented | 49 |
| AluSx1_dup96916 | Multi | No | — | — | SINE | Alu | chr19: 1925084-1925396 | − | 44 | 313 | SCAMP4 | Proximal | 0 | Not documented | 2450 |
| L2b_dup89911 | Uniquely | No | — | — | LINE | L2 | chr19: 19292859-19293219 | + | 130 | 361 | MEF2BNB | Proximal | 0 | Not documented | 51 |
| MIRb_dup208838 | Multi | No | — | — | SINE | MIR | chr19: 19313892-19314023 | + | 130 | 132 | NR2CZAP | Proximal | 0 | Not documented | 52 |
| MIRb_dup208849 | Multi/ | No | — | — | SINE | MIR | chr19: 19362120-19362374 | − | 140 | 255 | NCAN | Proximal | 0 | Not documented | 53 |
| | Uniquely | | | | | | | | | | | | | | |
| L1M3a_dup458 | Uniquely | No | — | — | LINE | Other L1 | chr19: 20045795-20046291 | + | 160 | 497 | ZNF93 | Proximal | 0 | Not documented | 54 |
| AluSx_dup125337 | Multi | No | — | — | SINE | Alu | chr19: 2072158-2072452 | + | 51 | 295 | MOB3A | Proximal | 0 | Not documented | 55 |
| FLAM_C_dup19851 | Multi | No | — | — | SINE | Alu | chr19: 2072639-2072758 | + | 60 | 120 | MOB3A | Proximal | 0 | Not documented | 56 |
| L1PBa_dup2080 | Uniquely | No | — | — | LINE | L1PA\|B\|x | chr19: 21454082-21456445 | − | 70 | 2364 | ZNF708 | Distal | 17518 | Not documented | 57 |
| L1PB2_dup2680 | Uniquely | No | — | — | LINE | L1PA\|B\|x | chr19: 21460758-21462263 | − | 70 | 1506 | ZNF708 | Distal | 11700 | Not documented | 58 |
| AluSc8_dup19773 | Uniquely | No | — | — | SINE | Alu | chr19: 21701357-21701659 | − | 46 | 303 | ZNF429 | Proximal | 0 | Not documented | 59 |
| LTR76_dup168 | Uniquely | No | — | — | LTR | ERV1 | chr19: 21701909-21702560 | + | 60 | 652 | ZNF429 | Proximal | 0 | Not documented | 60 |
| BSR/Beta_dup1564 | Uniquely | No | — | — | Other | Other | chr19: 22026603-22026931 | + | 120 | 329 | ZNF43 | Proximal | 0 | Not documented | 61 |
| | | | | | | Repeats | | | | | | | | | |
| AluJr4_dup16057 | Multi | No | — | — | SINE | Alu | chr19: 2233590-2233757 | + | 87 | 168 | PLEKHJ1 | Proximal | 0 | Not documented | 62 |
| L1MA8_dup10466 | Multi | No | — | — | LINE | Other L1 | chr19: 22877394-22877624 | − | 110 | 231 | ZNF492 | Distal | 26923 | Not documented | 63 |
| AluSx1_dup99732 | Multi | No | — | — | SINE | Alu | chr19: 22877697-22877946 | − | 47 | 250 | ZNF492 | Distal | 27226 | Not documented | 64 |
| AluY_dup109544 | Multi | No | — | — | SINE | Alu | chr19: 23542024-23542308 | − | 19 | 285 | ZNF91 | Proximal | 0 | Not documented | 65 |
| LTR70_dup129 | Multi | No | — | — | LTR | ERV1 | chr19: 23855533-23856476 | − | 51 | 944 | ZNF675 | Proximal | 0 | Not documented | 66 |
| AluSx1_dup99816 | Multi | No | — | — | SINE | Alu | chr19: 23993809-23994118 | − | 47 | 310 | RPSAP58 | Proximal | 0 | Not documented | 67 |
| HERV3-int_dup263 | Multi | No | — | — | LTR | ERV1 | chr19: 24001565-24004302 | − | 73 | 2738 | RPSAP58 | Proximal | 0 | EVE annotated | 68 |
| LTR6A_dup228 | Multi | No | — | — | LTR | ERV1 | chr19: 24009970-24009999 | − | 15 | 30 | RPSAP58 | Proximal | 0 | Not documented | 69 |
| LTR10B_dup62 | Multi | No | — | — | LTR | ERV1 | chr19: 24100679-24101097 | − | 67 | 419 | ZNF726 | Proximal | 0 | Not documented | 70 |
| HERVK14-int_{—} | Multi | No | — | — | LTR | ERVK | chr19: 24138536-24139324 | − | 140 | 789 | ZNF726 | Distal | 10576 | EVE annotated | 71 |
| dup344 | | | | | | | | | | | | | | | |
| SVA_C_dup261 | Multi | No | — | — | Other | SVA | chr19: 24146230-24147935 | + | 21 | 1706 | ZNF726 | Distal | 18270 | Not documented | 72 |
| BSR/Beta_dup1884 | Multi | No | — | — | Other | Other | chr19: 24238718-24240453 | − | 110 | 1736 | ZNF254 | Proximal | 0 | Not documented | 73 |
| | | | | | | Repeats | | | | | | | | | |
| BSR/Beta_dup1887 | Multi | No | — | — | Other | Other | chr19: 24241510-24242942 | − | 110 | 1433 | ZNF254 | Proximal | 0 | Not documented | 74 |
| | | | | | | Repeats | | | | | | | | | |
| MER4E1_dup958 | Multi | No | — | — | LTR | ERV1 | chr19: 24265482-24265627 | + | 40 | 146 | ZNF254 | Proximal | 0 | Not documented | 2475 |
| MER1A_dup3035 | Multi | No | — | — | DNA | DNA | chr19: 24356525-24357021 | − | 74 | 497 | ZNF254 | Distal | 43872 | Not documented | 76 |
| ALR/Alpha_dup1169 | Multi | No | — | — | Other | Other | chr19: 24488848-24494826 | + | 64 | 5979 | ZNF254 | Distal | 176195 | Not documented | 77 |
| | | | | | | Repeats | | | | | | | | | |
| L1PA4_dup11340 | Uniquely | No | — | — | LINE | L1PA\|B\|x | chr19: 28186347-28192495 | − | 33 | 6149 | UQCRFS1 | Distal | 1505679 | EVE annotated | 78 |
| LTR22C_dup368 | Uniquely | No | — | — | LTR | ERVK | chr19: 28236732-28237193 | + | 100 | 462 | UQCRFS1 | Distal | 1460981 | Not documented | 79 |
| L1ME4a_dup40628 | Multi | No | — | — | LINE | Other L1 | chr19: 30106305-30106629 | + | 160 | 325 | POP4 | Proximal | 0 | Not documented | 80 |
| MER2_dup8714 | Multi | No | — | — | DNA | DNA | chr19: 30192144-30192487 | − | 85 | 344 | C19orf12 | Proximal | 0 | Not documented | 81 |
| Tigger1_dup11269 | Multi | No | — | — | DNA | DNA | chr19: 30192561-30192706 | + | 74 | 146 | C19orf12 | Proximal | 0 | Not documented | 82 |
| Tigger1_dup11270 | Multi | No | — | — | DNA | DNA | chr19: 30192695-30192904 | + | 70 | 210 | C19orf12 | Proximal | 0 | Not documented | 83 |
| Tigger13a_dup2934 | Multi | No | — | — | DNA | DNA | chr19: 32975623-32976061 | + | 120 | 439 | DPY19L3 | Proximal | 0 | Not documented | 84 |
| AluSg_dup37590 | Multi | No | — | — | SINE | Alu | chr19: 33203283-33203594 | − | 44 | 312 | NUDT19 | Proximal | 0 | Not documented | 85 |
| AluSx1_dup100093 | Uniquely | No | — | — | SINE | Alu | chr19: 33469772-33470065 | + | 63 | 294 | RHPN2 | Proximal | 0 | Not documented | 86 |
| AluJb_dup131698 | Multi | No | — | — | SINE | Alu | chr19: 34891933-34892128 | − | 67 | 196 | RP11-618P17.4 | Proximal | 0 | Not documented | 87 |
| AluY_dup109813 | Multi/ | No | — | — | SINE | Alu | chr19: 34892129-34892424 | − | 35 | 296 | RP11-618P17.4 | Proximal | 0 | Not documented | 88 |
| | Uniquely | | | | | | | | | | | | | | |
| AluJb_dup131699 | Multi | No | — | — | SINE | Alu | chr19: 34892425-34892543 | − | 67 | 119 | RP11-618P17.4 | Proximal | 0 | Not documented | 89 |
| AluJo_dup65780 | Multi | No | — | — | SINE | Alu | chr19: 34892553-34892713 | − | 100 | 161 | RP11-618P17.4 | Proximal | 0 | Not documented | 90 |
| AluY_dup109814 | Multi | No | — | — | SINE | Alu | chr19: 34892723-34893012 | − | 25 | 290 | RP11-618P17.4 | Proximal | 0 | Not documented | 91 |
| MER51A_dup887 | Multi | No | — | — | LTR | ERV1 | chr19: 34893105-34893406 | + | 82 | 302 | RP11-618P17.4 | Proximal | 0 | Not documented | 92 |
| LTR12_dup527 | Uniquely | No | — | — | LTR | ERV1 | chr19: 35166562-35167201 | − | 51 | 640 | SCGB2B2 | Proximal | 0 | Not documented | 93 |
| AluSz_dup89569 | Multi/ | No | — | — | SINE | Alu | chr19: 35169057-35169134 | − | 47 | 78 | ZNF302 | Proximal | 0 | Not documented | 94 |
| | Uniquely | | | | | | | | | | | | | | |
| AluSx3_dup26791 | Multi | No | — | — | SINE | Alu | chr19: 35169135-35169355 | + | 47 | 221 | ZNF302 | Proximal | 0 | Not documented | 95 |
| AluJr_dup70791 | Multi | No | — | — | SINE | Alu | chr19: 35469602-35469907 | − | 64 | 306 | ZNF792 | Distal | 14650 | Not documented | 96 |
| MLT1E2_dup3676 | Multi | No | — | — | LTR | ERVL- | chr19: 35573164-35573729 | − | 110 | 566 | HPN | Distal | 15690 | Not documented | 97 |
| | | | | | | MaLR | | | | | | | | | |
| L2a_dup158183 | Multi | No | — | — | LINE | L2 | chr19: 35641320-35641989 | − | 140 | 670 | FXYD7 | Proximal | 0 | Not documented | 98 |
| FLAM_C_dup20527 | Multi | No | — | — | SINE | Alu | chr19: 35791939-35792072 | − | 75 | 134 | MAG | Proximal | 0 | Not documented | 99 |
| MER49_dup1250 | Multi | No | — | — | LTR | ERV1 | chr19: 35890087-35890709 | + | 57 | 623 | GPR42 | Distal | 26233 | Not documented | 2500 |
| Charlie1b_dup2830 | Multi | No | — | — | DNA | DNA | chr19: 35903621-35903683 | + | 93 | 63 | FFAR2 | Distal | 31127 | Not documented | 1 |
| LTR42_dup256 | Multi | No | — | — | LTR | ERVL | chr19: 35929462-35929904 | + | 72 | 443 | FFAR2 | Distal | 4906 | Not documented | 2 |
| L2a_dup158232 | Multi | No | — | — | LINE | L2 | chr19: 35963265-35963538 | + | 130 | 274 | KRTDAP | Distal | 14689 | Not documented | 3 |
| AluSx_dup129936 | Multi | No | — | — | SINE | Alu | chr19: 36010989-36011260 | + | 47 | 272 | SBSN | Distal | 3010 | Not documented | 4 |
| MIRb_dup209371 | Multi | No | — | — | SINE | MIR | chr19: 36024931-36025074 | − | 170 | 144 | GAPDHS | Proximal | 0 | Not documented | 5 |
| MER4D1_dup1408 | Multi | No | — | — | LTR | ERV1 | chr19: 36030200-36030383 | − | 52 | 184 | GAPDHS | Proximal | 0 | Not documented | 6 |
| L2b_dup90313 | Multi | No | — | — | LINE | L2 | chr19: 36039808-36039951 | − | 110 | 144 | ATP4A | Proximal | 995 | Not documented | 7 |
| L1M5_dup59948 | Multi | No | — | — | LINE | Other L1 | chr19: 36081071-36081415 | − | 150 | 345 | HAUS5 | Distal | 22232 | Not documented | 8 |
| AluSz_dup89640 | Multi | No | — | — | SINE | Alu | chr19: 36106937-36107232 | + | 49 | 296 | HAUS5 | Proximal | 0 | Not documented | 9 |
| L1MC5_dup19047 | Multi | No | — | — | LINE | Other L1 | chr19: 36117047-36117213 | + | 120 | 16 | HAUS5 | Proximal | 797 | Not documented | 10 |
| AluSq_dup19843 | Multi | No | — | — | SINE | Alu | chr19: 36233992-36234304 | + | 50 | 313 | U2AF1L4 | Proximal | 0 | Not documented | 11 |
| MSR1_dup14 | Multi | No | — | — | Other | Other | chr19: 36236916-36237169 | − | 130 | 254 | PSENEN | Proximal | 0 | Not documented | 12 |
| | | | | | | Repeats | | | | | | | | | |
| AluYf5_dup163 | Multi | No | — | — | SINE | Alu | chr19: 36313157-36313461 | + | 21 | 305 | NPHS1 | Distal | 3406 | Not documented | 13 |
| L2_dup53144 | Multi | No | — | — | LINE | L2 | chr19: 36506527-36506611 | − | 160 | 85 | CLIP3 | Proximal | 0 | Not documented | 14 |
| AluJb_dup131922 | Multi | No | — | — | SINE | Alu | chr19: 36716652-36716971 | − | 61 | 320 | ZNF146 | Proximal | 0 | Not documented | 15 |
| L2_dup53148 | Multi | No | — | — | LINE | L2 | chr19: 36717009-36717172 | + | 150 | 164 | ZNF146 | Proximal | 0 | Not documented | 16 |
| L1ME3C_dup11672 | Multi | No | — | — | LINE | Other L1 | chr19: 36728417-36728601 | + | 140 | 185 | ZNF146 | Proximal | 0 | Not documented | 17 |
| L1ME3C_dup11673 | Multi | No | — | — | LINE | Other L1 | chr19: 36728637-36728827 | + | 140 | 191 | ZNF146 | Proximal | 0 | Not documented | 18 |
| L1ME3C_dup11674 | Multi | No | — | — | LINE | Other L1 | chr19: 36728928-36729357 | + | 120 | 430 | ZNF146 | Proximal | 0 | Not documented | 19 |
| L1ME4a_dup40728 | Multi | No | — | — | LINE | Other L1 | chr19: 36729375-36729490 | + | 110 | 116 | ZNF146 | Proximal | 0 | Not documented | 20 |
| LTR6B_dup141 | Multi/ | No | — | — | LTR | ERV1 | chr19: 36815775-36816331 | − | 25 | 557 | ZFP14 | Distal | 10832 | Not documented | 21 |
| | Uniquely | | | | | | | | | | | | | | |
| HERVS71-int_{—} | Multi | No | — | — | LTR | ERV1 | chr19: 36816332-36816467 | − | 47 | 136 | ZFP14 | Distal | 10696 | EVE annotated | 22 |
| dup153 | | | | | | | | | | | | | | | |
| HERVS71-int_{—} | Multi | No | — | — | LTR | ERV1 | chr19: 36816444-36818252 | − | 45 | 1809 | ZFP14 | Distal | 8911 | EVE annotated | 23 |
| dup154 | | | | | | | | | | | | | | | |
| AluSp_dup45165 | Multi | No | — | — | SINE | Alu | chr19: 37002145-37002269 | + | 25 | 125 | ZNF260 | Proximal | 0 | Not documented | 24 |
| L2c_dup133108 | Uniquely | No | — | — | LINE | L2 | chr19: 37318551-37318659 | − | 140 | 109 | ZNF790 | Proximal | 0 | Not documented | 2525 |
| AluSp_dup45198 | Multi | No | — | — | SINE | Alu | chr19: 37346350-37346653 | − | 48 | 304 | ZNF345 | Proximal | 0 | Not documented | 26 |
| AluY_dup109992 | Uniquely | No | — | — | SINE | Alu | chr19: 37815674-37815978 | − | 58 | 305 | HKR1 | Proximal | 0 | Not documented | 27 |
| LTR27_dup395 | Multi | No | — | — | LTR | ERV1 | chr19: 37854939-37855554 | + | 100 | 616 | HKR1 | Proximal | 0 | Not documented | 28 |
| L2a_dup158372 | Uniquely | No | — | — | LINE | L2 | chr19: 37955668-37956356 | + | 120 | 689 | ZNF569 | Proximal | 0 | Not documented | 29 |
| AluSx_dup130424 | Multi | No | — | — | SINE | Alu | chr19: 39150560-39150861 | − | 53 | 302 | ACTN4 | Proximal | 0 | Not documented | 30 |
| MIRb_dup209521 | Multi | No | — | — | SINE | MIR | chr19: 39423432-39423477 | + | 120 | 46 | MRPS12 | Proximal | 0 | Not documented | 31 |
| MER91B_dup1464 | Multi | No | — | — | DNA | DNA | chr19: 39423583-39423661 | − | 120 | 79 | MRPS12 | Proximal | 0 | Not documented | 32 |
| AluJb_dup132262 | Multi | No | — | — | SINE | Alu | chr19: 39432899-39433202 | − | 61 | 304 | CTC-360G5.8 | Proximal | 0 | Not documented | 33 |
| MLT2C1_dup2402 | Multi | No | — | — | LTR | ERVL | chr19: 39889660-39889891 | − | 96 | 232 | MED29 | Proximal | 0 | Not documented | 34 |
| AluSx3_dup26923 | Multi | No | — | — | SINE | Alu | chr19: 39889893-39890188 | + | 34 | 296 | MED29 | Proximal | 0 | Not documented | 35 |
| ERVL-int_dup759 | Multi | No | — | — | LTR | ERVL | chr 19: 39890196-39890343 | − | 89 | 148 | MED29 | Proximal | 0 | Not documented | 36 |
| L2a_dup158600 | Multi | No | — | — | LINE | L2 | chr19: 39890628-39890715 | − | 99 | 88 | MED29 | Proximal | 0 | Not documented | 37 |
| FLAM_C_dup20625 | Uniquely | No | — | — | SINE | Alu | chr19: 40022613-40022763 | + | 42 | 151 | EID2B | Proximal | 0 | Not documented | 38 |
| AluSz_dup90093 | Uniquely | No | — | — | SINE | Alu | chr19: 40976208-40976513 | − | 54 | 306 | SPTBN4 | Proximal | 0 | Not documented | 39 |
| L1MEg2_dup696 | Multi | No | — | — | LINE | Other L1 | chr19: 41246580-41246617 | + | 38 | 38 | ITPKC | Proximal | 0 | Not documented | 40 |
| LTR41_dup1653 | Multi | No | — | — | LTR | ERVL | chr19: 41937306-41937575 | − | 110 | 270 | ATP5SL | Proximal | 0 | Not documented | 41 |
| LTR33_dup8680 | Multi | No | — | — | LTR | ERVL | chr19: 41937777-41937847 | − | 130 | 71 | ATP5SL | Proximal | 0 | Not documented | 42 |
| LTR33A_dup1553 | Multi | No | — | — | LTR | ERVL | chr19: 42882260-42882539 | − | 100 | 280 | MEGF8 | Proximal | 0 | Not documented | 43 |
| L2_dup53309 | Uniquely | No | — | — | LINE | L2 | chr19: 44645088-44645378 | + | 130 | 291 | ZNF234 | Proximal | 333 | Not documented | 44 |
| L2a_dup158958 | Multi | No | — | — | LINE | L2 | chr19: 44679260-44679408 | − | 140 | 149 | ZNF226 | Proximal | 0 | Not documented | 45 |
| MER91A_dup3383 | Uniquely | No | — | — | DNA | DNA | chr19: 44721747-44721914 | + | 160 | 168 | ZNF227 | Proximal | 0 | Not documented | 46 |
| MSR1_dup98 | Multi | No | — | — | Other | Other | chr19: 45404109-45404218 | − | 65 | 110 | TOMM40 | Proximal | 0 | Not documented | 47 |
| | | | | | | Repeats | | | | | | | | | |
| MIR_dup163590 | Multi | No | — | — | SINE | MIR | chr19: 45953278-45953433 | − | 120 | 156 | ERCC1 | Proximal | 0 | Not documented | 48 |
| AluSx_dup131291 | Multi | No | — | — | SINE | Alu | chr19: 45953746-45954057 | + | 35 | 312 | ERCC1 | Proximal | 0 | Not documented | 49 |
| L2b_dup90912 | Multi | No | — | — | LINE | L2 | chr19: 45979620-45979982 | − | 140 | 363 | ERCC1 | Proximal | 0 | Not documented | 2550 |
| AluSx1_dup101257 | Multi | No | — | — | SINE | Alu | chr19: 45979992-45980318 | + | 45 | 327 | ERCC1 | Proximal | 0 | Not documented | 51 |
| AluJr_dup71536 | Multi | No | — | — | SINE | Alu | chr19: 45980768-45980930 | + | 64 | 163 | ERCC1 | Proximal | 0 | Not documented | 52 |
| L2a_dup159110 | Uniquely | No | — | — | LINE | L2 | chr19: 45982448-45982545 | − | 100 | 98 | ERCC1 | Proximal | 363 | Not documented | 53 |
| MIR3_dup85063 | Multi | No | — | — | SINE | MIR | chr19: 46286802-46286872 | + | 140 | 71 | AC011530.4 | Proximal | 0 | Not documented | 54 |
| MIR3_dup85064 | Multi | No | — | — | SINE | MIR | chr19: 46287891-46287981 | + | 100 | 91 | AC011530.4 | Proximal | 0 | Not documented | 55 |
| AluSg_dup37996 | Uniquely | No | — | — | SINE | Alu | chr19: 46568567-46568863 | − | 51 | 297 | IGFL4 | Proximal | 0 | Not documented | 56 |
| L2a_dup159395 | Multi/ | No | — | — | LINE | L2 | chr19: 48244965-48245119 | − | 100 | 155 | EHD2 | Proximal | 0 | Not documented | 57 |
| | Uniquely | | | | | | | | | | | | | | |
| AluSx_dup131733 | Multi | No | — | — | SINE | Alu | chr19: 48245536-48245827 | − | 44 | 292 | EHD2 | Proximal | 0 | Not documented | 58 |
| AluSz_dup90846 | Multi | No | — | — | SINE | Alu | chr19: 48956595-48956898 | − | 50 | 304 | GRWD1 | Proximal | 0 | Not documented | 59 |
| MSR1_dup140 | Multi | No | — | — | Other | Other | chr19: 49337598-49339036 | − | 35 | 1439 | HSD17B14 | Proximal | 0 | Not documented | 60 |
| | | | | | | Repeats | | | | | | | | | |
| HERVIP10FH- | Multi | No | — | — | LTR | ERV1 | chr19: 49371679-49371929 | − | 91 | 251 | PLEKHA4 | Proximal | 0 | Not documented | 61 |
| int_dup634 | | | | | | | | | | | | | | | |
| MIRc_dup97194 | Multi | No | — | — | SINE | MIR | chr19: 50138730-50138777 | − | 47 | 48 | RRAS | Proximal | 0 | Not documented | 62 |
| AluSx1_dup101944 | Multi | No | — | — | SINE | Alu | chr19: 50320899-50321208 | + | 35 | 310 | FUZ | Proximal | 267 | Not documented | 63 |
| AluSz_dup91011 | Multi | No | — | — | SINE | Alu | chr19: 50410791-50411088 | − | 35 | 298 | NUP62 | Proximal | 0 | Not documented | 64 |
| AluSc8_dup20164 | Uniquely | No | — | — | SINE | Alu | chr19: 51399531-51399827 | − | 51 | 297 | KLK4 | Distal | 9782 | Not documented | 65 |
| L3_dup42953 | Multi | No | — | — | LINE | CR1 | chr19: 51853606-51853668 | + | 120 | 63 | ETFB | Proximal | 0 | Not documented | 66 |
| L2b_dup91336 | Multi | No | — | — | LINE | L2 | chr19: 52719061-52719091 | − | 59 | 31 | PPP2R1A | Proximal | 0 | Not documented | 67 |
| AluSx3_dup27195 | Uniquely | No | — | — | SINE | Alu | chr19: 52794730-52795029 | + | 42 | 300 | ZNF766 | Proximal | 0 | Not documented | 68 |
| AluJo_dup67002 | Multi | No | — | — | SINE | Alu | chr19: 52850779-52851075 | − | 89 | 29 | ZNF610 | Proximal | 0 | Not documented | 69 |
| AluJo_dup67006 | Uniquely | No | — | — | SINE | Alu | chr19: 52877717-52877888 | − | 77 | 172 | ZNF880 | Proximal | 0 | Not documented | 70 |
| AluSx_dup132452 | Uniquely | No | — | — | SINE | Alu | chr19: 52882650-52882995 | − | 54 | 346 | ZNF880 | Proximal | 0 | Not documented | 71 |
| AluSq2_dup50992 | Uniquely | No | — | — | SINE | Alu | chr19: 53060524-53060817 | − | 55 | 294 | ZNF701 | Proximal | 0 | Not documented | 72 |
| AluSq2_dup50999 | Multi | No | — | — | SINE | Alu | chr19: 53147986-53148284 | + | 59 | 299 | ZNF83 | Proximal | 0 | Not documented | 73 |
| AluSp_dup46022 | Multi | No | — | — | SINE | Alu | chr19: 53745177-53745480 | − | 36 | 304 | ZNF677 | Proximal | 0 | Not documented | 74 |
| AluY_dup111393 | Uniquely | No | — | — | SINE | Alu | chr19: 54609501-54609775 | − | 30 | 275 | NDUFA3 | Proximal | 0 | Not documented | 2575 |
| L2c_dup133599 | Multi | No | — | — | LINE | L2 | chr19: 54697391-54697519 | + | 160 | 129 | TSEN34 | Proximal | 0 | Not documented | 76 |
| MIRc_dup97306 | Multi | No | — | — | SINE | MIR | chr19: 56154492-56154590 | + | 150 | 99 | ZNF580 | Proximal | 0 | Not documented | 77 |
| MIRb_dup210808 | Multi | No | — | — | SINE | MIR | chr19: 56154607-56154714 | + | 130 | 108 | ZNF580 | Proximal | 0 | Not documented | 78 |
| MIRc_dup96183 | Multi | No | — | — | SINE | MIR | chr19: 5688214-5688265 | − | 92 | 52 | HSD11B1L | Proximal | 0 | Not documented | 79 |
| AluJb_dup134530 | Multi | No | — | — | SINE | Alu | chr19: 56909114-56909335 | − | 76 | 222 | ZNF583 | Proximal | 1 | Not documented | 80 |
| LTR9_dup1893 | Multi | No | — | — | LTR | ERV1 | chr19: 56909336-56909959 | − | 78 | 624 | ZNF583 | Proximal | 0 | Not documented | 81 |
| MER83_dup424 | Multi | No | — | — | LTR | ERV1 | chr19: 56910100-56910609 | + | 71 | 510 | ZNF583 | Proximal | 0 | Not documented | 82 |
| L1MA5_dup4253 | Uniquely | No | — | — | LINE | Other L1 | chr19: 56961072-56962956 | + | 65 | 1885 | ZNF667 | Proximal | 0 | Not documented | 83 |
| L2_dup53542 | Multi/ | No | — | — | LINE | L2 | chr19: 57005757-57005861 | + | 120 | 105 | ZNF471 | Distal | 13352 | Not documented | 84 |
| | Uniquely | | | | | | | | | | | | | | |
| L1MB3_dup16104 | Multi/ | No | — | — | LINE | Other L1 | chr19: 57005910-57006359 | − | 65 | 450 | ZNF471 | Distal | 12854 | Not documented | 85 |
| | Uniquely | | | | | | | | | | | | | | |
| LTR47B_dup341 | Multi/ | No | — | — | LTR | ERVL | chr19: 57006434-57006889 | + | 91 | 456 | ZNF471 | Distal | 12324 | Not documented | 86 |
| | Uniquely | | | | | | | | | | | | | | |
| L1ME4a_dup40868 | Multi | No | — | — | LINE | Other L1 | chr19: 57321527-57321661 | + | 110 | 135 | PEG3 | Proximal | 0 | Not documented | 87 |
| AluJr_dup72175 | Multi | No | — | — | SINE | Alu | chr19: 57808674-57809027 | − | 81 | 354 | ZNF460 | Distal | 3738 | Not documented | 88 |
| L1MA5_dup4261 | Multi | No | — | — | LINE | Other L1 | chr19: 57809028-57809835 | − | 75 | 808 | ZNF460 | Distal | 4092 | Not documented | 89 |
| MLT1J-int_dup1400 | Multi | No | — | — | LTR | ERVL- | chr19: 57819695-57819859 | + | 130 | 165 | ZNF543 | Distal | 12019 | Not documented | 90 |
| | | | | | | MaLR | | | | | | | | | |
| L1MB2_dup8465 | Multi | No | — | — | LINE | Other L1 | chr19: 57911243-57911790 | − | 78 | 548 | ZNF548 | Proximal | 0 | Not documented | 91 |
| L1M1_dup9071 | Multi/ | No | — | — | LINE | Other L1 | chr19: 57975158-57977873 | − | 94 | 2716 | AC004076.9 | Proximal | | Not documented | 92 |
| | Uniquely | | | | | | | | | | | | | | |
| L1M4b_dup6079 | Multi/ | No | — | — | LINE | Other L1 | chr19: 58005800-58006136 | − | 110 | 337 | AC003005.4 | Proximal | 0 | Not documented | 93 |
| | Uniquely | | | | | | | | | | | | | | |
| HERVK3-int_dup295 | Uniquely | No | — | — | LTR | ERVK | chr19: 58024740-58029437 | + | 78 | 4698 | ZNF773 | Proximal | 0 | EVE annotated | 94 |
| AluJo_dup67244 | Uniquely | No | — | — | SINE | Alu | chr19: 58268225-58268503 | − | 100 | 279 | ZNF776 | Proximal | 0 | Not documented | 95 |
| HERVK14-int_{—} | Uniquely | No | — | — | LTR | ERVK | chr19: 58412503-58414029 | + | 100 | 1527 | ZNF417 | Proximal | 0 | EVE annotated | 96 |
| dup357 | | | | | | | | | | | | | | | |
| MER112_dup4161 | Multi/ | No | — | — | DNA | DNA | chr19: 58595262-58595487 | − | 120 | 226 | ZNF135 | Proximal | 0 | Not documented | 97 |
| | Uniquely | | | | | | | | | | | | | | |
| AluJo_dup67263 | Uniquely | No | — | — | SINE | Alu | chr19: 58599004-58599306 | − | 63 | 303 | ZSCAN18 | Proximal | 0 | Not documented | 98 |
| AluJr_dup72221 | Multi/ | No | — | — | SINE | Alu | chr19: 58759629-58759898 | − | 76 | 270 | ZNF544 | Proximal | 0 | Not documented | 99 |
| | Uniquely | | | | | | | | | | | | | | |
| L1MC1_dup12533 | Multi | No | — | — | LINE | Other L1 | chr19: 58759899-58760049 | − | 90 | 151 | ZNF544 | Proximal | 0 | Not documented | 2600 |
| MER51A_dup903 | Multi/ | No | — | — | LTR | ERV1 | chr19: 58760050-58760579 | − | 59 | 530 | ZNF544 | Proximal | 0 | Not documented | 1 |
| | Uniquely | | | | | | | | | | | | | | |
| HERV4_I-int_{—} | Multi/ | No | — | — | LTR | ERV1 | chr19: 58760581-58761957 | − | 130 | 1377 | ZNF544 | Proximal | 0 | Not documented | 2 |
| dup324 | Uniquely | | | | | | | | | | | | | | |
| MLT1D_dup19275 | Multi | No | — | — | LTR | ERVL- | chr19: 58770769-58770984 | − | 110 | 216 | ZNF544 | Proximal | 0 | Not documented | 3 |
| | | | | | | MaLR | | | | | | | | | |
| MER41C_dup780 | Uniquely | No | — | — | LTR | ERV1 | chr19: 58789310-58789824 | + | 28 | 515 | CTD-3138B18.4 | Proximal | 0 | Not documented | 4 |
| HERVK3-int_dup296 | Multi | No | — | — | LTR | ERVK | chr19: 58817484-58821622 | + | 84 | 4139 | AC010642.1 | Proximal | 0 | EVE annotated | 5 |
| HERVK3-int_dup299 | Multi | No | — | — | LTR | ERVK | chr19: 58826017-58826205 | + | 83 | 189 | AC010642.1 | Proximal | 0 | Not documented | 6 |
| LTR3_dup82 | Multi | No | — | — | LTR | ERVK | chr19: 58826206-58826633 | + | 78 | 428 | AC010642.1 | Proximal | 0 | Not documented | 7 |
| L1MB7_dup21723 | Multi/ | No | — | — | LINE | Other L1 | chr19: 58826693-58827054 | − | 75 | 362 | AC010642.1 | Proximal | 131 | Not documented | 8 |
| | Uniquely | | | | | | | | | | | | | | |
| L2a_dup160239 | Multi | No | — | — | LINE | L2 | chr19: 58836601-58836739 | + | 160 | 139 | ZSCAN22 | Proximal | 1647 | Not documented | 9 |
| L2c_dup133706 | Uniquely | No | — | — | LINE | L2 | chr19: 58865718-58865979 | − | 110 | 262 | ZNF497 | Proximal | 0 | Not documented | 10 |
| MLT1L_dup11302 | Multi | No | — | — | LTR | ERVL- | chr19: 58929481-58929578 | + | 130 | 98 | ZNF584 | Proximal | 0 | Not documented | 11 |
| | | | | | | MaLR | | | | | | | | | |
| MIRc_dup96199 | Multi | No | — | — | SINE | MIR | chr19: 5901421-5901501 | + | 150 | 81 | NDUFA11 | Proximal | 0 | Not documented | 12 |
| MIRb_dup210958 | Multi | No | — | — | SINE | MIR | chr19: 59073306-59073409 | − | 130 | 104 | MZF1 | Proximal | 0 | Not documented | 13 |
| AluYc_dup7855 | Multi | No | — | — | SINE | Alu | chr19: 59093573-59093619 | + | 9.5 | 47 | MZF1 | Distal | 8632 | Not documented | 14 |
| MER31-int_dup1932 | Multi | No | — | — | LTR | ERV1 | chr19: 59110887-59111333 | − | 96 | 447 | MZF1 | Distal | 25946 | Not documented | 15 |
| MER58A_dup12388 | Uniquely | No | — | — | DNA | DNA | chr19: 6043509-6043724 | + | 91 | 216 | RFX2 | Proximal | 0 | Not documented | 16 |
| LTR87_dup598 | Multi | No | — | — | LTR | ERVL | chr19: 6211036-6211120 | − | 130 | 85 | MLLT1 | Proximal | 1847 | Not documented | 17 |
| MIRc_dup96211 | Multi | No | — | — | SINE | MIR | chr19: 6414481-6414548 | − | 120 | 68 | KHSRP | Proximal | 0 | Not documented | 18 |
| AluSz_dup87652 | Multi | No | — | — | SINE | Alu | chr19: 8439377-8439674 | + | 63 | 298 | ANGPTL4 | Proximal | 121 | Not documented | 19 |
| MLT1F_dup3894 | Multi | No | — | — | LTR | ERVL- | chr19: 8921341-8921879 | + | 120 | 539 | ZNF558 | Proximal | 0 | Not documented | 20 |
| | | | | | | MaLR | | | | | | | | | |
| AluSg_dup36525 | Multi | No | — | — | SINE | Alu | chr19: 893586-893894 | − | 55 | 309 | MED16 | Proximal | 369 | Not documented | 21 |
| MER20_dup15075 | Multi | No | — | — | DNA | DNA | chr19: 9272786-9272989 | + | 110 | 204 | ZNF317 | Proximal | 0 | Not documented | 22 |
| L1MA9_dup15711 | Uniquely | No | — | — | LINE | Other L1 | chr19: 9483546-9486561 | − | 71 | 3016 | ZNF177 | Proximal | 0 | Not documented | 23 |
| AluSz_dup87771 | Multi | No | — | — | SINE | Alu | chr19: 9638662-9638942 | − | 45 | 281 | ZNF426 | Proximal | 0 | Not documented | 24 |
| L1MC4_dup26965 | Multi | No | — | — | LINE | Other L1 | chr19: 9671573-9672280 | + | 130 | 708 | ZNF121 | Proximal | 0 | Not documented | 2625 |
| HERVI-int_dup134 | Uniquely | No | — | — | LTR | ERV1 | chr19: 9738586-9745083 | + | 120 | 6498 | C19orf82 | Proximal | 0 | EVE annotated | 26 |
| MER11C_dup788 | Multi | No | — | — | LTR | ERVK | chr19: 9759673-9760731 | + | 26 | 1059 | ZNF562 | Proximal | 0 | Not documented | 27 |
| MER74B_dup103 | Uniquely | No | — | — | LTR | ERVL | chr2: 100890910-100891377 | + | 96 | 468 | LONRF2 | Proximal | 0 | Not documented | 28 |
| HERVL74-int_dup43 | Uniquely | No | — | — | LTR | ERVL | chr2: 100892579-100894938 | + | 96 | 2360 | LONRF2 | Proximal | 0 | Not documented | 29 |
| L1ME3_dup1101 | Uniquely | No | — | — | LINE | Other L1 | chr2: 100932810-100934846 | − | 110 | 2037 | LONRF2 | Proximal | 0 | Not documented | 30 |
| L4_dup1771 | Multi | No | — | — | LINE | RTE | chr2: 101206638-101206703 | − | 140 | 66 | PDCL3 | Distal | 13442 | Not documented | 31 |
| L1ME3A_dup1240 | Uniquely | No | — | — | LINE | Other L1 | chr2: 10297461-10297904 | − | 120 | 444 | C2orf48 | Proximal | 0 | Not documented | 32 |
| HUERS-P1-int_{—} | Uniquely | No | — | — | LTR | ERV1 | chr2: 104093127-104095347 | − | 100 | 2221 | TMEM182 | Distal | 632776 | Not documented | 33 |
| dup54 | | | | | | | | | | | | | | | |
| HUERS-P1-int_{—} | Uniquely | No | — | — | LTR | ERV1 | chr2: 104095662-104096155 | − | 100 | 494 | TMEM182 | Distal | 635311 | Not documented | 34 |
| dup55 | | | | | | | | | | | | | | | |
| HUERS-P1-int_{—} | Multi/ | No | — | — | LTR | ERV1 | chr2: 104096158-104096839 | − | 57 | 682 | TMEM182 | Distal | 635807 | Not documented | 35 |
| dup56 | Uniquely | | | | | | | | | | | | | | |
| MER41B_dup286 | Multi/ | No | — | — | LTR | ERV1 | chr2: 104096840-104097468 | − | 52 | 629 | TMEM182 | Distal | 636489 | Not documented | 36 |
| | Uniquely | | | | | | | | | | | | | | |
| MIR_dup21689 | Uniquely | No | — | — | SINE | MIR | chr2: 105123589-105123804 | − | 110 | 216 | POU3F3 | Distal | 348166 | Not documented | 37 |
| MER58A_dup1389 | Multi/ | No | — | — | DNA | DNA | chr2: 105423895-105424112 | − | 93 | 218 | POU3F3 | Distal | 47858 | Not documented | 38 |
| | Uniquely | | | | | | | | | | | | | | |
| L1ME3A_dup1757 | Multi/ | No | — | — | LINE | Other L1 | chr2: 105428626-105429037 | + | 100 | 412 | POU3F3 | Distal | 42933 | Not documented | 39 |
| | Uniquely | | | | | | | | | | | | | | |
| L4_dup1792 | Multi | No | — | — | LINE | RTE | chr2: 105450668-105450721 | + | 100 | 54 | POU3F3 | Distal | 21249 | Not documented | 40 |
| AluSx1_dup9184 | Multi | No | — | — | SINE | Alu | chr2: 10830259-10830566 | + | 28 | 308 | NOL10 | Proximal | 159 | Not documented | 41 |
| AluY_dup13922 | Multi/ | No | — | — | SINE | Alu | chr2: 109004252-109004556 | − | 30 | 305 | SULT1C4 | Proximal | 0 | Not documented | 42 |
| | Uniquely | | | | | | | | | | | | | | |
| L2_dup7205 | Multi/ | No | — | — | LINE | L2 | chr2: 109004760-109005210 | + | 150 | 451 | SULT1C4 | Proximal | 248 | Not documented | 43 |
| | Uniquely | | | | | | | | | | | | | | |
| Tigger4b_dup304 | Multi | No | — | — | DNA | DNA | chr2: 110301378-110301693 | − | 67 | 316 | sept-10 | Proximal | 0 | Not documented | 44 |
| L1M4c_dup623 | Uniquely | No | — | — | LINE | Other L1 | chr2: 110890391-110892793 | + | 77 | 2403 | NPHP1 | Proximal | 0 | Not documented | 45 |
| L1M2_dup910 | Multi | No | — | — | LINE | Other L1 | chr2: 112089275-112090485 | − | 70 | 1211 | BCL2L11 | Distal | 163252 | Not documented | 46 |
| L1MDa_dup812 | Multi | No | — | — | LINE | Other L1 | chr2: 112101490-112101806 | − | 120 | 317 | BCL2L11 | Distal | 175467 | Not documented | 47 |
| AluYa5_dup481 | Multi | No | — | — | SINE | Alu | chr2: 112242227-112242518 | + | 7.7 | 292 | ANAPC1 | Distal | 281331 | Not documented | 48 |
| AluYa5_dup482 | Multi | No | — | — | SINE | Alu | chr2: 112399486-112399797 | + | 4.5 | 312 | ANAPC1 | Distal | 124052 | Not documented | 49 |
| L2b_dup13547 | Multi | No | — | — | LINE | L2 | chr2: 114023120-114023387 | + | 140 | 268 | PAX8 | Proximal | 0 | Not documented | 2650 |
| MER31-int_dup245 | Multi | No | — | — | LTR | ERV1 | chr2: 114368825-114369271 | + | 100 | 447 | RABL2A | Distal | 15536 | Not documented | 51 |
| AluSc5_dup792 | Multi | No | — | — | SINE | Alu | chr2: 114400213-114400502 | − | 28 | 290 | RABL2A | Proximal | 0 | Not documented | 52 |
| L2_dup5465 | Uniquely | No | — | — | LINE | L2 | chr2: 11465679-11467698 | − | 160 | 2020 | ROCK2 | Proximal | 0 | Not documented | 53 |
| MER131_dup65 | Multi | No | — | — | SINE | SINE | chr2: 118867293-118867429 | − | 160 | 137 | INSIG2 | Proximal | | Not documented | 54 |
| L2a_dup22113 | Multi | No | — | — | LINE | L2 | chr2: 120125421-120125545 | + | 130 | 125 | DB | Proximal | 0 | Not documented | 55 |
| MIRb_dup31476 | Uniquely | No | — | — | SINE | MIR | chr2: 120439465-120439674 | + | 160 | 210 | TMEM177 | Proximal | 0 | Not documented | 56 |
| L1ME1_dup4016 | Uniquely | No | — | — | LINE | Other L1 | chr2: 121512727-121514170 | − | 110 | 1444 | GLI2 | Proximal | 0 | Not documented | 57 |
| AluJo_dup8985 | Multi | No | — | — | SINE | Alu | chr2: 122523898-122524029 | + | 52 | 132 | TSN | Proximal | 0 | Not documented | 58 |
| L1MC_dup1343 | Multi | No | — | — | LINE | Other L1 | chr2: 128608253-128608349 | + | 110 | 97 | POLR2D | Proximal | 0 | Not documented | 59 |
| L3_dup6525 | Multi | No | — | — | LINE | CR1 | chr2: 130930188-130930242 | − | 110 | 55 | SMPD4 | Proximal | 0 | Not documented | 60 |
| L1MA9_dup1775 | Multi | No | — | — | LINE | Other L1 | chr2: 131104497-131105081 | − | 75 | 585 | IMP4 | Proximal | 0 | Not documented | 61 |
| MLT1A0_dup2518 | Multi | No | — | — | LTR | ERVL- | chr2: 131105083-131105416 | + | 78 | 334 | IMP4 | Proximal | 0 | Not documented | 62 |
| | | | | | | MaLR | | | | | | | | | |
| MIRc_dup11187 | Uniquely | No | — | — | SINE | MIR | chr2: 13144002-13144092 | + | 150 | 91 | TRIB2 | Distal | 261143 | Not documented | 63 |
| AluSq_dup2730 | Multi | No | — | — | SINE | Alu | chr2: 132227382-132227675 | − | 43 | 294 | MZT2A | Proximal | 0 | Not documented | 64 |
| AluSx3_dup3778 | Multi | No | — | — | SINE | Alu | chr2: 132553830-132554149 | − | 58 | 320 | C2orf27B | Proximal | 0 | Not documented | 65 |
| TAR1_dup21 | Multi | No | — | — | Other | Other | chr2: 132560215-132560696 | + | 71 | 482 | C2orf27B | Proximal | 982 | Not documented | 66 |
| | | | | | | Repeats | | | | | | | | | |
| L1PB_dup175 | Multi | No | — | — | LINE | L1PA\|B\|x | chr2: 132561092-132561536 | − | 37 | 445 | C2orf27B | Proximal | 1859 | Not documented | 67 |
| TAR1_dup22 | Multi | No | — | — | Other | Other | chr2: 132570743-132571233 | − | 76 | 491 | C2orf27B | Distal | 11510 | Not documented | 68 |
| | | | | | | Repeats | | | | | | | | | |
| L1MA4_dup1100 | Multi | No | — | — | LINE | Other L1 | chr2: 132709922-132711670 | − | 83 | 1749 | C2orf27B | Distal | 150689 | Not documented | 69 |
| AluSq2_dup7015 | Multi | No | — | — | SINE | Alu | chr2: 132713093-132713371 | − | 55 | 279 | C2orf27B | Distal | 153860 | Not documented | 70 |
| L1M4_dup2000 | Multi | No | — | — | LINE | Other L1 | chr2: 132732715-132735569 | + | 120 | 2855 | ANKRD30BL | Distal | 169596 | Not documented | 71 |
| MLT1A0_dup2527 | Multi | No | — | — | LTR | ERVL- | chr2: 132755503-132755888 | + | 96 | 386 | ANKRD30BL | Distal | 149277 | Not documented | 72 |
| | | | | | | MaLR | | | | | | | | | |
| L1PA16_dup1488 | Multi | No | — | — | LINE | L1PA\|B\|x | chr2: 132760103-132761264 | − | 63 | 1162 | ANKRD30BL | Distal | 143901 | Not documented | 73 |
| MER77_dup131 | Multi | No | — | — | LTR | ERVL | chr2: 132764598-132765208 | − | 120 | 611 | ANKRD30BL | Distal | 139957 | Not documented | 74 |
| CER_dup5 | Multi | No | — | — | Other | Other | chr2: 132826613-132836513 | + | 57 | 9901 | ANKRD30BL | Distal | 68652 | Not documented | 2675 |
| | | | | | | Repeats | | | | | | | | | |
| L1MA9_dup1783 | Multi | No | — | — | LINE | Other L1 | chr2: 132846401-132848533 | − | 100 | 2133 | ANKRD30BL | Distal | 56632 | Not documented | 76 |
| MSTB_dup1003 | Multi | No | — | — | LTR | ERVL- | chr2: 132852207-132852618 | + | 100 | 412 | ANKRD30BL | Distal | 52547 | Not documented | 77 |
| | | | | | | MaLR | | | | | | | | | |
| L2a_dup22714 | Multi | No | — | — | LINE | L2 | chr2: 132892485-132892812 | − | 150 | 328 | ANKRD30BL | Distal | 12353 | Not documented | 78 |
| LTR10A_dup42 | Multi | No | — | — | LTR | ERV1 | chr2: 132905124-132905636 | − | 79 | 513 | ANKRD30BL | Proximal | 0 | Not documented | 79 |
| AluSp_dup6548 | Multi | No | — | — | SINE | Alu | chr2: 132939403-132939716 | + | 51 | 314 | ANKRD30BL | Proximal | 0 | Not documented | 80 |
| ALR/Alpha_dup67 | Multi | No | — | — | Other | Other | chr2: 132966375-132977849 | + | 63 | 11475 | ANKRD30BL | Proximal | 0 | Not documented | 81 |
| | | | | | | Repeats | | | | | | | | | |
| MER45C_dup161 | Multi | No | — | — | DNA | DNA | chr2: 133011227-133011381 | − | 110 | 155 | ANKRD30BL | Proximal | 0 | Not documented | 82 |
| AluY_dup14974 | Multi | No | — | — | SINE | Alu | chr2: 133015979-133016280 | + | 42 | 302 | ANKRD30BL | Proximal | 438 | Not documented | 83 |
| AluSq2_dup7020 | Multi | No | — | — | SINE | Alu | chr2: 133050673-133050964 | + | 64 | 292 | ANKRD30BL | Distal | 35132 | Not documented | 84 |
| L1MD2_dup1230 | Multi | No | — | — | LINE | Other L1 | chr2: 133086622-133087681 | + | 95 | 1060 | ANKRD30BL | Distal | 71081 | Not documented | 85 |
| LTR50_dup311 | Multi | No | — | — | LTR | ERVL | chr2: 133102676-133102912 | − | 110 | 237 | GPR39 | Distal | 71236 | Not documented | 86 |
| TAR1_dup23 | Multi | No | — | — | Other | Other | chr2: 133109768-133110293 | − | 78 | 526 | GPR39 | Distal | 63855 | Not documented | 87 |
| | | | | | | Repeats | | | | | | | | | |
| L1PA8_dup897 | Multi | No | — | — | LINE | L1PA\|B\|x | chr2: 133121635-133123314 | + | 45 | 1680 | GPR39 | Distal | 50834 | Not documented | 88 |
| L1M7_dup519 | Multi | No | — | — | LINE | Other L1 | chr2: 133209279-133210061 | − | 110 | 783 | GPR39 | Proximal | 0 | Not documented | 89 |
| MER46C_dup335 | Multi | No | — | — | DNA | DNA | chr2: 133283601-133283892 | + | 110 | 292 | GPR39 | Proximal | 0 | Not documented | 90 |
| L1PA8_dup902 | Multi | No | — | — | LINE | L1PA\|B\|x | chr2: 133295853-133297084 | − | 75 | 1232 | GPR39 | Proximal | 0 | Not documented | 91 |
| LTR21A_dup12 | Multi | No | — | — | LTR | ERV1 | chr2: 133315194-133315601 | − | 100 | 408 | GPR39 | Proximal | 0 | Not documented | 92 |
| L2c_dup21674 | Multi | No | — | — | LINE | L2 | chr2: 135816931-135817062 | − | 140 | 132 | RAB3GAP1 | Proximal | 0 | Not documented | 93 |
| L1ME4a_dup5649 | Multi | No | — | — | LINE | Other L1 | chr2: 136541589-136541848 | + | 130 | 260 | UBXN4 | Proximal | 0 | Not documented | 94 |
| AluJo_dup9260 | Multi | No | — | — | SINE | Alu | chr2: 136664809-136664874 | − | 55 | 66 | DARS | Proximal | 0 | Not documented | 95 |
| L1PB4_dup743 | Multi | No | — | — | LINE | L1PA\|B\|x | chr2: 142128774-142134140 | − | 99 | 5367 | LRP1B | Proximal | 0 | Not documented | 96 |
| L1MB1_dup683 | Multi | No | — | — | LINE | Other L1 | chr2: 142174794-142176736 | − | 77 | 1943 | LRP1B | Proximal | 0 | Not documented | 97 |
| L1M1_dup996 | Multi | No | — | — | LINE | Other L1 | chr2: 142500123-142502203 | − | 77 | 2081 | LRP1B | Proximal | 0 | Not documented | 98 |
| L1MC4_dup3672 | Uniquely | No | — | — | LINE | Other L1 | chr2: 142629808-142630716 | + | 130 | 909 | LRP1B | Proximal | 0 | Not documented | 99 |
| MER20_dup1813 | Multi | No | — | — | DNA | DNA | chr2: 142767633-142767842 | + | 120 | 210 | LRP1B | Proximal | 0 | Not documented | 2700 |
| AluY_dup15301 | Multi | No | — | — | SINE | Alu | chr2: 142773281-142773581 | + | 21 | 301 | LRP1B | Proximal | 0 | Not documented | 1 |
| L2a_dup23255 | Uniquely | No | — | — | LINE | L2 | chr2: 142776923-142777679 | + | 130 | 757 | LRP1B | Proximal | 0 | Not documented | 2 |
| AluSx_dup18329 | Multi | No | — | — | SINE | Alu | chr2: 142837069-142837378 | − | 59 | 310 | LRP1B | Proximal | 0 | Not documented | 3 |
| MLT1A0_dup1709 | Multi/ | No | — | — | LTR | ERVL- | chr2: 14789881-14790233 | + | 84 | 353 | AC011897.1 | Proximal | 0 | Not documented | 4 |
| | Uniquely | | | | | MaLR | | | | | | | | | |
| L1ME4a_dup5867 | Uniquely | No | — | — | LINE | Other L1 | chr2: 152236140-152236628 | − | 110 | 489 | TNFAIP6 | Proximal | 0 | Not documented | 5 |
| L1MC3_dup1629 | Uniquely | No | — | — | LINE | Other L1 | chr2: 159853521-159854516 | + | 110 | 996 | TANC1 | Proximal | 0 | Not documented | 6 |
| AluJr4_dup2572 | Uniquely | No | — | — | SINE | Alu | chr2: 159922638-159922939 | + | 65 | 302 | TANC1 | Proximal | 0 | Not documented | 7 |
| L1MB7_dup3075 | Uniquely | No | — | — | LINE | Other L1 | chr2: 159962612-159963402 | − | 83 | 791 | TANC1 | Proximal | 0 | Not documented | 8 |
| AluJo_dup9591 | Multi/ | No | — | — | SINE | Alu | chr2: 159989856-159990034 | − | 74 | 179 | TANC1 | Proximal | 0 | Not documented | 9 |
| | Uniquely | | | | | | | | | | | | | | |
| MARNA_dup477 | Multi | No | — | — | DNA | DNA | chr2: 160088189-160088272 | − | 100 | 84 | TANC1 | Proximal | 0 | Not documented | 10 |
| L1MA6_dup687 | Uniquely | No | — | — | LINE | Other L1 | chr2: 166709932-166711873 | − | 84 | 1942 | TTC21B | Distal | 2113 | Not documented | 11 |
| L1MEg1_dup147 | Uniquely | No | — | — | LINE | Other L1 | chr2: 168196835-168197676 | + | 130 | 842 | XIRP2 | Distal | 80573 | Not documented | 12 |
| AluY_dup16043 | Multi/ | No | — | — | SINE | Alu | chr2: 168728402-168728711 | − | 10 | 310 | B3GALT1 | Proximal | 0 | Not documented | 13 |
| | Uniquely | | | | | | | | | | | | | | |
| AluSq_dup1863 | Uniquely | No | — | — | SINE | Alu | chr2: 1693273-1693571 | + | 56 | 299 | PXDN | Proximal | 0 | Not documented | 14 |
| THE1A-int_dup206 | Uniquely | No | — | — | LTF | ERVL- | chr2: 170543726-170545414 | − | 40 | 1689 | CCDC173 | Proximal | 0 | Not documented | 15 |
| | | | | | | MaLR | | | | | | | | | |
| AluJb_dup20216 | Uniquely | No | — | — | SINE | Alu | chr2: 170662478-170662774 | − | 77 | 297 | SSB | Proximal | 0 | Not documented | 16 |
| L1PA6_dup772 | Uniquely | No | — | — | LINE | L1PA\|B\|x | chr2: 170672860-170674431 | + | 29 | 1572 | METTL5 | Proximal | 0 | Not documented | 17 |
| L2_dup8175 | Uniquely | No | — | — | LINE | L2 | chr2: 171866495-171867112 | − | 160 | 618 | TLK1 | Proximal | 0 | Not documented | 18 |
| MLT1A_dup1186 | Uniquely | No | — | — | LTR | ERVL- | chr2: 171874528-171874865 | + | 87 | 338 | TLK1 | Proximal | 0 | Not documented | 19 |
| | | | | | | MaLR | | | | | | | | | |
| AluSq2_dup7552 | Uniquely | No | — | — | SINE | Alu | chr2: 175261243-175261543 | + | 33 | 301 | SCRN3 | Proximal | 0 | Not documented | 20 |
| MIRb_dup35373 | Multi | No | — | — | SINE | MIR | chr2: 176793021-176793157 | − | 130 | 137 | KIAA1715 | Proximal | 0 | Not documented | 21 |
| AluJr_dup10246 | Uniquely | No | — | — | SINE | Alu | chr2: 177494960-177495185 | + | 66 | 226 | MTX2 | Distal | 292208 | Not documented | 22 |
| AluJo_dup10012 | Multi | No | — | — | SINE | Alu | chr2: 178087254-178087517 | + | 78 | 264 | HNRNPA3 | Proximal | 0 | Not documented | 23 |
| L1M3f_dup81 | Uniquely | No | — | — | LINE | Other L1 | chr2: 179288966-179291775 | − | 79 | 2810 | PRKRA | Distal | 4367 | Not documented | 24 |
| MER58C_dup385 | Multi/ | No | — | — | DNA | DNA | chr2: 183642015-183642232 | − | 110 | 218 | DNAJC10 | Proximal | 0 | Not documented | 2725 |
| | Uniquely | | | | | | | | | | | | | | |
| AluJo_dup10082 | Multi | No | — | — | SINE | Alu | chr2: 183800703-183800993 | + | 94 | 291 | NCKAP1 | Proximal | 0 | Not documented | 26 |
| AluSz_dup13399 | Uniquely | No | — | — | SINE | Alu | chr2: 183801599-183801870 | + | 45 | 272 | NCKAP1 | Proximal | 0 | Not documented | 27 |
| L1PA15_dup1016 | Multi | No | — | — | LINE | L1PA\|B\|x | chr2: 183858255-183858586 | + | 50 | 332 | NCKAP1 | Proximal | 0 | Not documented | 28 |
| L1ME2z_dup992 | Multi/ | No | — | — | LINE | Other L1 | chr2: 183858630-183858744 | + | 100 | 115 | NCKAP1 | Proximal | 0 | Not documented | 29 |
| | Uniquely | | | | | | | | | | | | | | |
| MIRb_dup35814 | Multi | No | — | — | SINE | MIR | chr2: 183858985-183859083 | + | 120 | 99 | NCKAP1 | Proximal | 0 | Not documented | 30 |
| AluSz_dup13403 | Multi/ | No | — | — | SINE | Alu | chr2: 183859084-183859394 | + | 53 | 311 | NCKAP1 | Proximal | 0 | Not documented | 31 |
| | Uniquely | | | | | | | | | | | | | | |
| AluSx3_dup4169 | Multi | No | — | — | SINE | Alu | chr2: 186810550-186810854 | + | 46 | 305 | FSIP2 | Distal | 112534 | Not documented | 32 |
| L1MA7_dup1035 | Uniquely | No | — | — | LINE | Other L1 | chr2: 186976070-186979187 | − | 110 | 3118 | FSIP2 | Distal | 278054 | Not documented | 33 |
| AluJb_dup20970 | Uniquely | No | — | — | SINE | Alu | chr2: 187321463-187321770 | + | 87 | 308 | ZC3H15 | Distal | 29114 | Not documented | 34 |
| L1MB2_dup1142 | Uniquely | No | — | — | LINE | Other L1 | chr2: 187391400-187392919 | − | 72 | 1520 | ZC3H15 | Distal | 17311 | Not documented | 35 |
| L1MA3_dup663 | Uniquely | No | — | — | LINE | Other L1 | chr2: 187563241-187564956 | − | 45 | 1716 | FAM171B | Proximal | 0 | Not documented | 36 |
| AluJb_dup21009 | Multi/ | No | — | — | SINE | Alu | chr2: 188329954-188330229 | + | 68 | 276 | TFPI | Proximal | 0 | Not documented | 37 |
| | Uniquely | | | | | | | | | | | | | | |
| L2_dup8495 | Multi | No | — | — | LINE | L2 | chr2: 188330317-188330419 | − | 180 | 103 | TFPI | Proximal | 0 | Not documented | 38 |
| MER58B_dup970 | Multi | No | — | — | DNA | DNA | chr2: 191069508-191069812 | − | 91 | 305 | HIBCH | Proximal | 0 | Not documented | 39 |
| LTR33_dup1242 | Uniquely | No | — | — | LTR | ERVL | chr2: 197714630-197715038 | + | 130 | 409 | PGAP1 | Proximal | 0 | Not documented | 40 |
| MER124_dup104 | Multi | No | — | — | DNA | DNA | chr2: 198436330-198436573 | − | 120 | 244 | RFTN2 | Proximal | 0 | Not documented | 41 |
| L1MDb_dup144 | Multi/ | No | — | — | LINE | Other L1 | chr2: 200820720-200820848 | + | 120 | 129 | C2orf47 | Proximal | 0 | Not documented | 42 |
| | Uniquely | | | | | | | | | | | | | | |
| AluSx_dup19969 | Multi | No | — | — | SINE | Alu | chr2: 201343618-201343925 | − | 53 | 308 | SPATS2L | Proximal | 0 | Not documented | 43 |
| MLT1K_dup2297 | Multi | No | — | — | LTR | ERVL- | chr2: 201343926-201344291 | − | 120 | 366 | SPATS2L | Proximal | 0 | Not documented | 44 |
| | | | | | | MaLR | | | | | | | | | |
| MER65A_dup226 | Uniquely | No | — | — | LTR | ERV1 | chr2: 202485152-202485584 | − | 58 | 433 | TMEM237 | Proximal | 0 | Not documented | 45 |
| AluJb_dup21578 | Uniquely | No | — | — | SINE | Alu | chr2: 202624764-202625059 | + | 77 | 296 | ALS2 | Proximal | 0 | Not documented | 46 |
| Tigger14a_dup186 | Multi | No | — | — | DNA | DNA | chr2: 203070992-203071098 | − | 87 | 107 | SUMO1 | Proximal | 0 | Not documented | 47 |
| Tigger3b_dup933 | Uniquely | Nc | — | — | DNA | DNA | chr2: 204216583-204217852 | + | 58 | 1270 | ABI2 | Proximal | 0 | Not documented | 48 |
| AluSg_dup6073 | Multi | No | — | — | SINE | Alu | chr2: 204249547-204249854 | − | 40 | 308 | ABI2 | Proximal | 0 | Not documented | 49 |
| AluYf4_dup216 | Multi | No | — | — | SINE | Alu | chr2: 204249860-204250153 | − | 26 | 294 | ABI2 | Proximal | 0 | Not documented | 2750 |
| AluSx1_dup15091 | Uniquely | No | — | — | SINE | Alu | chr2: 204251364-204251662 | − | 55 | 299 | ABI2 | Proximal | 0 | Not documented | 51 |
| AluSx_dup20281 | Multi | No | — | — | SINE | Alu | chr2: 204253124-204253407 | + | 42 | 284 | ABI2 | Proximal | 0 | Not documented | 52 |
| AluSz_dup13960 | Multi | No | — | — | SINE | Alu | chr2: 204264803-204265111 | − | 66 | 309 | ABI2 | Proximal | 0 | Not documented | 53 |
| AluYb8_dup382 | Multi/ | No | — | — | SINE | Alu | chr2: 204273553-204273860 | − | 5.9 | 308 | ABI2 | Proximal | 0 | Not documented | 54 |
| | Uniquely | | | | | | | | | | | | | | |
| MIRb_dup36842 | Uniquely | No | — | — | SINE | MIR | chr2: 204274416-204274642 | − | 150 | 227 | ABI2 | Proximal | 0 | Not documented | 55 |
| AluSc_dup4951 | Multi | No | — | — | SINE | Alu | chr2: 204294200-204294493 | + | 40 | 294 | ABI2 | Proximal | 0 | Not documented | 56 |
| AluY_dup17396 | Uniquely | No | — | — | SINE | Alu | chr2: 205581347-205581644 | − | 27 | 298 | PARD3B | Proximal | 0 | Not documented | 57 |
| L1PREC2_dup1040 | Uniquely | No | — | — | LINE | L1PA\|B\|x | chr2: 205673053-205675966 | − | 50 | 2914 | PARD3B | Proximal | 0 | Not documented | 58 |
| LFSINE_Vert_{—} | Multi/ | No | — | — | SINE | SINE | chr2: 207025571-207025803 | − | 82 | 233 | EEF1B2 | Proximal | 0 | Not documented | 59 |
| dup156 | Uniquely | | | | | | | | | | | | | | |
| L1MD3_dup778 | Multi | No | — | — | LINE | Other L1 | chr2: 208446314-208447425 | − | 91 | 1112 | METTL21A | Proximal | 0 | Not documented | 60 |
| MIRb_dup37265 | Multi | No | — | — | SINE | MIR | chr2: 210885187-210885417 | + | 150 | 231 | RPE | Proximal | 0 | Not documented | 61 |
| MIRc_dup17730 | Multi | No | — | — | SINE | MIR | chr2: 210885450-210885603 | − | 150 | 154 | RPE | Proximal | 0 | Not documented | 62 |
| L1MA3_dup1243 | Uniquely | No | — | — | LINE | Other L1 | chr2: 210980701-210982390 | + | 65 | 1690 | KANSL1L | Proximal | 0 | Not documented | 63 |
| L2a_dup27009 | Uniquely | No | — | — | LINE | L2 | chr2: 213116035-213117449 | − | 130 | 1415 | ERBB4 | Proximal | 0 | Not documented | 64 |
| MLT1D_dup2742 | Uniquely | No | — | — | LTR | ERVL- | chr2: 213314942-213315420 | + | 100 | 479 | ERBB4 | Proximal | 0 | Not documented | 65 |
| | | | | | | MaLR | | | | | | | | | |
| L1MC1_dup2026 | Multi/ | No | — | — | LINE | Other L1 | chr2: 214182496-214182695 | + | 55 | 200 | SPAG16 | Proximal | 0 | Not documented | 66 |
| | Uniquely | | | | | | | | | | | | | | |
| MLT1F1_dup453 | Uniquely | No | — | — | LTR | ERVL- | chr2: 216978217-216978776 | − | 120 | 560 | XRCC5 | Proximal | 0 | Not documented | 67 |
| | | | | | | MaLR | | | | | | | | | |
| L1ME1_dup5076 | Uniquely | No | — | — | LINE | Other L1 | chr2: 218835791-218836411 | − | 95 | 621 | TNS1 | Proximal | 0 | Not documented | 68 |
| Charlie16a_dup339 | Multi/ | No | — | — | DNA | DNA | chr2: 220420495-220420613 | + | 130 | 119 | OBSL1 | Proximal | 0 | Not documented | 69 |
| | Uniquely | | | | | | | | | | | | | | |
| MER102c_dup482 | Multi/ | No | — | — | DNA | DNA | chr2: 220426475-220426797 | − | 130 | 323 | OBSL1 | Proximal | 0 | Not documented | 70 |
| | Uniquely | | | | | | | | | | | | | | |
| MIRb_dup38164 | Uniquely | No | — | — | SINE | MIR | chr2: 222390244-222390431 | + | 130 | 188 | EPHA4 | Proximal | 0 | Not documented | 71 |
| AluY_dup18114 | Uniquely | No | — | — | SINE | Alu | chr2: 225229337-225229635 | + | 21 | 299 | FAM124B | Distal | 13781 | Not documented | 72 |
| MER20_dup2199 | Uniquely | No | — | — | DNA | DNA | chr2: 231912221-231912408 | + | 110 | 188 | C2orf72 | Proximal | 0 | Not documented | 73 |
| MIR_dup17638 | Uniquely | No | — | — | SINE | MIR | chr2: 23614804-23615036 | + | 120 | 233 | KLHL29 | Proximal | 0 | Not documented | 74 |
| L2_dup9415 | Uniquely | No | — | — | LINE | L2 | chr2: 236847967-236849119 | + | 140 | 1153 | AGAP1 | Proximal | 0 | Not documented | 2775 |
| AluJo_dup11151 | Multi | No | — | — | SINE | Alu | chr2: 237996291-237996612 | + | 66 | 322 | COPS8 | Proximal | 0 | Not documented | 76 |
| L3_dup8122 | Multi | No | — | — | LINE | CR1 | chr2: 238007182-238007413 | − | 140 | 232 | COPS8 | Proximal | 0 | Not documented | 77 |
| L3_dup8154 | Multi | No | — | — | LINE | CR1 | chr2: 240961576-240961632 | − | 95 | 57 | NDUFA10 | Proximal | 0 | Not documented | 78 |
| L1ME3C_dup2043 | Uniquely | No | — | — | LINE | Other L1 | chr2: 242014221-242016008 | − | 140 | 1788 | MTERFD2 | Proximal | 0 | Not documented | 79 |
| LTR78_dup441 | Multi | No | — | — | LTR | ERV1 | chr2: 24390291-24390824 | − | 130 | 534 | RP11-507M3.1 | Proximal | 0 | Not documented | 80 |
| MIR3_dup10024 | Uniquely | No | — | — | SINE | MIR | chr2: 26468416-26468568 | − | 120 | 153 | HADHB | Proximal | 0 | Not documented | 81 |
| AluY_dup10707 | Uniquely | No | — | — | SINE | Alu | chr2: 27171445-27171745 | + | 32 | 301 | DPYSL5 | Proximal | 0 | Not documented | 82 |
| L2a_dup17654 | Multi | No | — | — | LINE | L2 | chr2: 27532541-27532573 | − | 55 | 33 | MPV17 | Proximal | 0 | Not documented | 83 |
| L4_dup1359 | Multi | No | — | — | LINE | RTE | chr2: 27873501-27873534 | − | 28 | 34 | GPN1 | Proximal | 0 | Not documented | 84 |
| L2c_dup17231 | Multi | No | — | — | LINE | L2 | chr2: 27885074-27885251 | − | 120 | 178 | SUPT7L | Proximal | 0 | Not documented | 85 |
| L1PB3_dup260 | Multi | No | — | — | LINE | L1PA\|B\|x | chr2: 27916099-27916572 | − | 95 | 474 | SLC4A1AP | Proximal | 0 | Not documented | 86 |
| L1ME3_dup855 | Multi | No | — | — | LINE | Other L1 | chr2: 28994217-28994900 | + | 120 | 684 | PPP1CB | Proximal | 0 | Not documented | 87 |
| Tigger12c_dup123 | Multi | No | — | — | DNA | DNA | chr2: 29024744-29024864 | + | 110 | 121 | PPP1CB | Proximal | 0 | Not documented | 88 |
| Charlie1a_dup569 | Uniquely | No | — | — | DNA | DNA | chr2: 32676532-32677577 | + | 98 | 1046 | BIRC6 | Proximal | 0 | Not documented | 89 |
| L1MB8_dup1309 | Multi | No | — | — | LINE | Other L1 | chr2: 37326766-37327285 | + | 64 | 520 | EIF2AK2 | Proximal | 0 | Not documented | 90 |
| AluSz_dup9540 | Multi | No | — | — | SINE | Alu | chr2: 37429507-37429847 | + | 50 | 341 | AC007390.5 | Proximal | 0 | Not documented | 91 |
| AluSc_dup3278 | Uniquely | No | — | — | SINE | Alu | chr2: 37431414-37431718 | − | 42 | 305 | AC007390.5 | Proximal | 0 | Not documented | 92 |
| AluJb_dup14925 | Multi | No | — | — | SINE | Alu | chr2: 37431719-37431768 | − | 56 | 50 | AC007390.5 | Proximal | 0 | Not documented | 93 |
| AluSc8_dup2037 | Uniquely | No | — | — | SINE | Alu | chr2: 38974225-38974283 | + | 110 | 59 | SRSF7 | Proximal | 0 | Not documented | 94 |
| L1MEd_dup1090 | Multi | No | — | — | LINE | Other L1 | chr2: 44545919-44546125 | + | 130 | 207 | PREPL | Proximal | 0 | Not documented | 95 |
| MIR_dup18758 | Multi | No | — | — | SINE | MIR | chr2: 44546126-44546196 | − | 130 | 71 | PREPL | Proximal | 0 | Not documented | 96 |
| L1MEd_dup1091 | Multi | Nc | — | — | LINE | Other L1 | chr2: 44546426-44546527 | + | 110 | 102 | PREPL | Proximal | 0 | Not documented | 97 |
| L1MEc_dup2141 | Uniquely | No | — | — | LINE | Other L1 | chr2: 46760713-46762438 | − | 130 | 1726 | ATP6V1E2 | Proximal | 0 | Not documented | 98 |
| L1PA16_dup1078 | Multi/ | No | — | — | LINE | L1PA\|B\|x | chr2: 46853079-46853966 | − | 49 | 888 | CRIPT | Proximal | 199 | Not documented | 99 |
| | Uniquely | | | | | | | | | | | | | | |
| L1PA16_dup1079 | Uniquely | No | — | — | LINE | L1PA\|B\|x | chr2: 46854272-46854580 | − | 43 | 309 | CRIPT | Proximal | 1392 | Not documented | 2800 |
| THE1A_dup416 | Multi | No | — | — | LTR | ERVL- | chr2: 47130142-47130492 | + | 25 | 351 | MCFD2 | Proximal | 0 | Not documented | 1 |
| | | | | | | MaLR | | | | | | | | | |
| THE1A-int_dup131 | Multi | No | — | — | LTR | ERVL- | chr2: 47130493-47132008 | + | 34 | 1516 | MCFD2 | Proximal | 0 | Not documented | 2 |
| | | | | | | MaLR | | | | | | | | | |
| THE1A_dup417 | Multi | No | — | — | LTR | ERVL- | chr2: 47132009-47132359 | + | 25 | 351 | MCFD2 | Proximal | 0 | Not documented | 3 |
| | | | | | | MaLR | | | | | | | | | |
| MIRb_dup27132 | Multi | No | — | — | SINE | MIR | chr2: 47399408-47399594 | + | 110 | 187 | CALM2 | Proximal | 0 | Not documented | 4 |
| L3_dup5310 | Multi | No | — | — | LINE | CR1 | chr2: 47637248-47637289 | − | 120 | 42 | MSH2 | Proximal | 0 | Not documented | 5 |
| AluYc_dup829 | Multi/ | No | — | — | SINE | Alu | chr2: 50844279-50844581 | − | 50 | 303 | NRXN1 | Proximal | 0 | Not documented | 6 |
| | Uniquely | | | | | | | | | | | | | | |
| Tigger15a_dup572 | Multi/ | No | — | — | DNA | DNA | chr2: 50910400-50910488 | + | 120 | 89 | NRXN1 | Proximal | 0 | Not documented | 7 |
| | Uniquely | | | | | | | | | | | | | | |
| AluY_dup11751 | Multi/ | No | — | — | SINE | Alu | chr2: 50910489-50910784 | + | 40 | 296 | NRXN1 | Proximal | 0 | Not documented | 8 |
| | Uniquely | | | | | | | | | | | | | | |
| Tigger4_dup174 | Multi/ | No | — | — | DNA | DNA | chr2: 51017787-51018122 | − | 91 | 336 | NRXN1 | Proximal | 0 | Not documented | 9 |
| | Uniquely | | | | | | | | | | | | | | |
| L4_dup1512 | Multi | No | — | — | LINE | RTE | chr2: 51018416-51018639 | + | 130 | 224 | NRXN1 | Proximal | 0 | Not documented | 10 |
| MIRc_dup13163 | Multi/ | No | — | — | SINE | MIR | chr2: 51061704-51061841 | + | 140 | 138 | NRXN1 | Proximal | 0 | Not documented | 11 |
| | Uniquely | | | | | | | | | | | | | | |
| MER5A_dup3171 | Uniquely | No | — | — | DNA | DNA | chr2: 51061914-51062007 | − | 140 | 94 | NRXN1 | Proximal | 0 | Not documented | 12 |
| L1MA7_dup676 | Uniquely | No | — | — | LINE | Other L1 | chr2: 51063342-51063894 | − | 69 | 553 | NRXN1 | Proximal | 0 | Not documented | 13 |
| THE1C_dup892 | Multi/ | No | — | — | LTR | ERVL- | chr2: 51605019-51605395 | + | 59 | 377 | NRXN1 | Distal | 345346 | Not documented | 14 |
| | Uniquely | | | | | MaLR | | | | | | | | | |
| L1MC2_dup554 | Uniquely | No | — | — | LINE | Other L1 | chr2: 51659870-51661759 | + | 110 | 1890 | NRXN1 | Distal | 400197 | Not documented | 15 |
| HERV17-int_dup93 | Uniquely | No | — | — | LTR | ERV1 | chr2: 53986012-53987331 | + | 28 | 1320 | ASB3 | Proximal | 0 | EVE annotated | 16 |
| MER1A_dup273 | Multi | No | — | — | DNA | DNA | chr2: 54184302-54184801 | − | 58 | 500 | PSME4 | Proximal | | Not documented | 17 |
| L1MC5_dup1722 | Multi | No | — | — | LINE | Other L1 | chr2: 54184802-54184945 | + | 100 | 144 | PSME4 | Proximal | 0 | Not documented | 18 |
| AluSz_dup10117 | Multi | No | — | — | SINE | Alu | chr2: 54185096-54185343 | + | 44 | 248 | PSME4 | Proximal | 0 | Not documented | 19 |
| AluSx_dup14849 | Multi | No | — | — | SINE | Alu | chr2: 54194508-54194816 | + | 44 | 309 | PSME4 | Proximal | 0 | Not documented | 20 |
| LTR7B_dup91 | Multi | No | — | — | LTR | ERV1 | chr2: 54200476-54200939 | + | 25 | 464 | ACYP2 | Proximal | 0 | Not documented | 21 |
| AluJo_dup7516 | Multi | No | — | — | SINE | Alu | chr2: 54277912-54278209 | − | 95 | 298 | ACYP2 | Proximal | 0 | Not documented | 22 |
| AluJo_dup7522 | Multi | No | — | — | SINE | Alu | chr2: 54379253-54379560 | − | 75 | 308 | ACYP2 | Proximal | 0 | Not documented | 23 |
| L1MC2_dup563 | Multi | No | — | — | LINE | Other L1 | chr2: 54379561-54379963 | − | 70 | 403 | ACYP2 | Proximal | 0 | Not documented | 24 |
| L1ME2_dup1332 | Multi | No | — | — | LINE | Other L1 | chr2: 55262271-55263455 | + | 100 | 1185 | RTN4 | Proximal | 0 | Not documented | 2825 |
| L1PREC2_dup656 | Multi | No | — | — | LINE | L1PA\|B\|x | chr2: 55268389-55269180 | + | 140 | 792 | RTN4 | Proximal | 0 | Not documented | 26 |
| L1PREC2_dup657 | Multi | No | — | — | LINE | L1PA\|B\|x | chr2: 55269261-55269588 | + | 130 | 328 | RTN4 | Proximal | 0 | Not documented | 27 |
| L2c_dup18570 | Multi | No | — | — | LINE | L2 | chr2: 55269888-55270110 | − | 150 | 223 | RTN4 | Proximal | 0 | Not documented | 28 |
| AluJb_dup15890 | Multi | No | — | — | SINE | Alu | chr2: 55270607-55270739 | + | 87 | 133 | RTN4 | Proximal | 0 | Not documented | 29 |
| AluSx_dup14905 | Multi | No | — | — | SINE | Alu | chr2: 55270740-55271048 | + | 53 | 309 | RTN4 | Proximal | 0 | Not documented | 30 |
| AluJo_dup7547 | Multi | No | — | — | SINE | Alu | chr2: 55271063-55271335 | + | 77 | 273 | RTN4 | Proximal | 0 | Not documented | 31 |
| AluJb_dup15892 | Multi | No | — | — | SINE | Alu | chr2: 55271336-55271563 | + | 87 | 228 | RTN4 | Proximal | 0 | Not documented | 32 |
| AluJr_dup7674 | Multi | No | — | — | SINE | Alu | chr2: 55271566-55271671 | + | 69 | 106 | RTN4 | Proximal | 0 | Not documented | 33 |
| L4_dup1530 | Uniquely | No | — | — | LINE | RTE | chr2: 55402211-55402737 | + | 160 | 527 | CLHC1 | Proximal | 0 | Not documented | 34 |
| L2a_dup19256 | Uniquely | No | — | — | LINE | L2 | chr2: 55457229-55458065 | − | 100 | 837 | CLHC1 | Proximal | 0 | Not documented | 35 |
| L1MC1_dup1338 | Uniquely | No | — | — | LINE | Other L1 | chr2: 55766100-55767864 | − | 51 | 1765 | CCDC104 | Proximal | 0 | Not documented | 36 |
| MER91C_dup127 | Multi | No | — | — | DNA | DNA | chr2: 5835430-5835520 | − | 140 | 91 | SOX11 | Proximal | 0 | Not documented | 37 |
| L1P2_dup165 | Uniquely | No | — | — | LINE | L1PA\|B\|x | chr2: 61248198-61249617 | + | 34 | 1420 | PEX13 | Proximal | 0 | Not documented | 38 |
| LTR6A_dup22 | Multi | No | — | — | LTR | ERV1 | chr2: 62373518-62373547 | − | 15 | 30 | COMMD1 | Proximal | 0 | Not documented | 39 |
| AluY_dup12368 | Uniquely | No | — | — | SINE | Alu | chr2: 68358047-68358349 | − | 35 | 303 | WDR92 | Proximal | 0 | Not documented | 40 |
| AluSz6_dup5053 | Multi | No | — | — | SINE | Alu | chr2: 68402411-68402692 | − | 58 | 282 | PNO1 | Proximal | 0 | Not documented | 41 |
| MER117_dup437 | Uniquely | No | — | — | DNA | DNA | chr2: 69474216-69474377 | − | 120 | 162 | ANTXR1 | Proximal | 0 | Not documented | 42 |
| Tigger14a_dup122 | Multi/ | No | — | — | DNA | DNA | chr2: 69475971-69476216 | − | 110 | 246 | ANTXR1 | Proximal | 0 | Not documented | 43 |
| | Uniquely | | | | | | | | | | | | | | |
| MER5A_dup3430 | Uniquely | No | — | — | DNA | DNA | chr2: 70507562-70507732 | − | 110 | 171 | PCYOX1 | Proximal | 0 | Not documented | 44 |
| L2b_dup12515 | Multi | No | — | — | LINE | L2 | chr2: 71213067-71213320 | − | 140 | 254 | AC007040.11 | Proximal | 0 | Not documented | 45 |
| L2b_dup12516 | Multi | No | — | — | LINE | L2 | chr2: 71213417-71214255 | − | 140 | 839 | TEX261 | Proximal | 0 | Not documented | 46 |
| AluSx3_dup3216 | Multi | No | — | — | SINE | Alu | chr2: 71638123-71638427 | − | 52 | 305 | ZNF638 | Proximal | 0 | Not documented | 47 |
| MIR3_dup11748 | Multi | No | — | — | SINE | MIR | chr2: 73170973-73171093 | − | 170 | 121 | SFXN5 | Proximal | 0 | Not documented | 48 |
| AluJo_dup8092 | Uniquely | No | — | — | SINE | Alu | chr2: 73744374-73744674 | − | 80 | 301 | ALMS1 | Proximal | 0 | Not documented | 49 |
| AluSq2_dup6064 | Multi | No | — | — | SINE | Alu | chr2: 73959557-73959855 | + | 41 | 299 | TPRKB | Proximal | 0 | Not documented | 2850 |
| L1MC4_dup3003 | Multi | No | — | — | LINE | Other L1 | chr2: 74089644-74089749 | − | 80 | 106 | STAMBP | Proximal | 0 | Not documented | 51 |
| L2_dup6663 | Uniquely | No | — | — | LINE | L2 | chr2: 74375489-74375767 | − | 160 | 279 | BOLA3 | Proximal | 369 | Not documented | 52 |
| L2c_dup19585 | Uniquely | No | — | — | LINE | L2 | chr2: 75325819-75326909 | + | 160 | 1091 | TACR1 | Proximal | 0 | Not documented | 53 |
| MamRep605_dup538 | Multi | No | — | — | Other | Other | chr2: 75882609-75882719 | − | 150 | 111 | MRPL19 | Proximal | 0 | Not documented | 54 |
| | | | | | | Repeats | | | | | | | | | |
| L1ME3B_dup898 | Multi | No | — | — | LINE | Other L1 | chr2: 75882720-75882998 | − | 120 | 279 | MRPL19 | Proximal | 0 | Not documented | 55 |
| 7SK_dup75 | Multi | No | — | — | Other | Other | chr2: 76672296-76672536 | − | 7.7 | 241 | LRRTM4 | Distal | 302310 | Not documented | 56 |
| | | | | | | Repeats | | | | | | | | | |
| AluSx1_dup11644 | Multi | No | — | — | SINE | Alu | chr2: 79067359-79067688 | − | 55 | 330 | REG3G | Distal | 185125 | Not documented | 57 |
| L2a_dup20561 | Uniquely | No | — | — | LINE | L2 | chr2: 80140092-80140813 | − | 150 | 722 | CTNNA2 | Proximal | 0 | Not documented | 58 |
| AluSz6_dup5231 | Uniquely | Nc | — | — | SINE | Alu | chr2: 80157081-80157368 | + | 58 | 288 | CTNNA2 | Proximal | 0 | Not documented | 59 |
| MER47A_dup297 | Uniquely | No | — | — | DNA | DNA | chr2: 80167825-80168094 | − | 71 | 270 | CTNNA2 | Proximal | 0 | Not documented | 60 |
| Charlie22a_dup144 | Multi | No | — | — | DNA | DNA | chr2: 80232949-80233128 | − | 120 | 180 | CTNNA2 | Proximal | 0 | Not documented | 61 |
| AluJr_dup8327 | Multi | No | — | — | SINE | Alu | chr2: 80254812-80255127 | + | 68 | 316 | CTNNA2 | Proximal | 0 | Not documented | 62 |
| AluJb_dup17147 | Multi | No | — | — | SINE | Alu | chr2: 80260409-80260724 | + | 72 | 316 | CTNNA2 | Proximal | 0 | Not documented | 63 |
| L1MC4a_dup2778 | Multi | No | — | — | LINE | Other L1 | chr2: 80260725-80261311 | + | 140 | 587 | CTNNA2 | Proximal | 0 | Not documented | 64 |
| MER5A1_dup1540 | Multi/ | No | — | — | DNA | DNA | chr2: 80280699-80280803 | − | 100 | 105 | CTNNA2 | Proximal | 0 | Not documented | 65 |
| | Uniquely | | | | | | | | | | | | | | |
| MLT1H2_dup458 | Uniquely | No | — | — | LTR | ERVL- | chr2: 80291568-80292050 | − | 110 | 483 | CTNNA2 | Proximal | 0 | Not documented | 66 |
| | | | | | | MaLR | | | | | | | | | |
| MLT1F_dup463 | Multi | No | — | — | LTR | ERVL- | chr2: 80293173-80293304 | + | 100 | 132 | CTNNA2 | Proximal | 0 | Not documented | 67 |
| | | | | | | MaLR | | | | | | | | | |
| L1MA8_dup977 | Multi | No | — | — | LINE | Other L1 | chr2: 80293364-80294456 | + | 55 | 1093 | CTNNA2 | Proximal | 0 | Not documented | 68 |
| L1PA4_dup1138 | Uniquely | No | — | — | LINE | L1PA\|B\|x | chr2: 80320128-80326280 | − | 21 | 6153 | CTNNA2 | Proximal | 0 | Not documented | 69 |
| MER58A_dup1294 | Uniquely | Nc | — | — | DNA | DNA | chr2: 80343187-80343392 | − | 100 | 206 | CTNNA2 | Proximal | 0 | Not documented | 70 |
| LTR16B1_dup108 | Uniquely | No | — | — | LTR | ERVL | chr2: 80356166-80356574 | − | 120 | 409 | CTNNA2 | Proximal | 0 | Not documented | 71 |
| MIR_dup20772 | Uniquely | No | — | — | SINE | MIR | chr2: 80356728-80356970 | − | 160 | 243 | CTNNA2 | Proximal | 0 | Not documented | 72 |
| MLT1C_dup1771 | Uniquely | No | — | — | LTR | ERVL- | chr2: 80360056-80360521 | + | 80 | 466 | CTNNA2 | Proximal | 0 | Not documented | 73 |
| | | | | | | MaLR | | | | | | | | | |
| L2b_dup12811 | Uniquely | No | — | — | LINE | L2 | chr2: 80401733-80402558 | + | 170 | 826 | CTNNA2 | Proximal | 0 | Not documented | 74 |
| AluSx1_dup11671 | Multi | No | — | — | SINE | Alu | chr2: 80416070-80416378 | − | 49 | 309 | CTNNA2 | Proximal | 0 | Not documented | 2875 |
| MIRb_dup29174 | Uniquely | No | — | — | SINE | MIR | chr2: 80424505-80424728 | − | 140 | 224 | CTNNA2 | Proximal | 0 | Not documented | 76 |
| THE1B-int_dup417 | Uniquely | No | — | — | LTR | ERVL- | chr2: 80430317-80431894 | − | 43 | 1578 | CTNNA2 | Proximal | 0 | Not documented | 77 |
| | | | | | | MaLR | | | | | | | | | |
| L1MEd_dup875 | Uniquely | No | — | — | LINE | Other L1 | chr2: 8582392-8582875 | + | 160 | 484 | ID2 | Distal | 236101 | Not documented | 78 |
| MER5A_dup3661 | Multi | No | — | — | DNA | DNA | chr2: 85835436-85835507 | − | 130 | 72 | C2orf68 | Proximal | 0 | Not documented | 79 |
| L1PREC2_dup754 | Multi | No | — | — | LINE | L1PA\|B\|x | chr2: 86730898-86731190 | + | 40 | 293 | CHMP3 | Proximal | 0 | Not documented | 80 |
| FLAM_C_dup2762 | Multi | No | — | — | SINE | Alu | chr2: 86731635-86731769 | − | 77 | 135 | CHMP3 | Proximal | 0 | Not documented | 81 |
| MIR_dup21071 | Multi | No | — | — | SINE | MIR | chr2: 86731882-86731958 | + | 110 | 77 | CHMP3 | Proximal | 0 | Not documented | 82 |
| TAR1_dup11 | Multi | No | — | — | Other | Other | chr2: 87575814-87576578 | + | 89 | 765 | PLGLB1 | Distal | 326840 | Not documented | 83 |
| | | | | | | Repeats | | | | | | | | | |
| L1MDa_dup656 | Multi | No | — | — | LINE | Other L1 | chr2: 87906120-87906436 | + | 120 | 317 | PLGLB2 | Distal | 141171 | Not documented | 84 |
| AluJr4_dup2238 | Uniquely | No | — | — | SINE | Alu | chr2: 88928047-88928340 | − | 70 | 294 | EIF2AK3 | Proximal | 954 | Not documented | 85 |
| TAR1_dup8 | Multi | No | — | — | Other | Other | chr2: 91750391-91751008 | − | 80 | 618 | RP11-685N3.1 | Distal | 1211995 | Not documented | 86 |
| | | | | | | Repeats | | | | | | | | | |
| MLT1H1_dup367 | Multi/ | No | — | — | LTR | ERVL- | chr2: 91805820-91806248 | − | 120 | 429 | RP11-685N3.1 | Distal | 1267424 | Not documented | 87 |
| | Uniquely | | | | | MaLR | | | | | | | | | |
| MLT1J2_dup786 | Multi/ | No | — | — | LTR | ERVL- | chr2: 91824937-91825072 | − | 140 | 136 | RP11-685N3.1 | Distal | 1286541 | Not documented | 88 |
| | Uniquely | | | | | MaLR | | | | | | | | | |
| L1MEc_dup2413 | Multi | No | — | — | LINE | Other L1 | chr2: 91838304-91839549 | − | 86 | 1246 | RP11-685N3.1 | Distal | 1299908 | Not documented | 89 |
| TAR1_dup14 | Multi | No | — | — | Other | Other | chr2: 91847958-91848503 | + | 74 | 546 | RP11-685N3.1 | Distal | 1309562 | Not documented | 90 |
| | | | | | | Repeats | | | | | | | | | |
| MIRb_dup23630 | Multi | No | — | — | SINE | MIR | chr2: 9562855-9563068 | − | 140 | 214 | ITGB1BP1 | Proximal | 0 | Not documented | 91 |
| AluJb_dup17648 | Multi/ | No | — | — | SINE | Alu | chr2: 96851847-96851916 | + | 52 | 70 | STARD7 | Proximal | 0 | Not documented | 92 |
| | Uniquely | | | | | | | | | | | | | | |
| MER106A_dup77 | Multi | No | — | — | DNA | DNA | chr2: 96937757-96937932 | + | 99 | 176 | CIAO1 | Proximal | 0 | Not documented | 93 |
| Charlie1a_dup729 | Multi | No | — | — | DNA | DNA | chr2: 96938088-96938161 | + | 78 | 74 | CIAO1 | Proximal | 0 | Not documented | 94 |
| AluSp_dup6023 | Multi | No | — | — | SINE | Alu | chr2: 96938273-96938563 | + | 39 | 291 | CIAO1 | Proximal | 0 | Not documented | 95 |
| Charlie1_dup243 | Multi | No | — | — | DNA | DNA | chr2: 96938564-96939120 | + | 89 | 557 | CIAO1 | Proximal | 0 | Not documented | 96 |
| AluSg4_dup899 | Multi | No | — | — | SINE | Alu | chr2: 96939121-96939424 | + | 35 | 304 | CIAO1 | Proximal | 35 | Not documented | 97 |
| Charlie1_dup244 | Multi | No | — | — | DNA | DNA | chr2: 96939425-96939664 | + | 89 | 240 | CIAO1 | Proximal | 339 | Not documented | 98 |
| AluSz6_dup5383 | Multi | Nc | — | — | SINE | Alu | chr2: 96939665-96939794 | + | 66 | 130 | SNRNP200 | Proximal | 281 | Not documented | 99 |
| L1PA7_dup1257 | Uniquely | No | — | — | LINE | L1PA\|B\|x | chr2: 98509366-98513152 | − | 39 | 3787 | TMEM131 | Proximal | 0 | EVE annotated | 2900 |
| MLT1K_dup1755 | Multi/ | No | — | — | LTR | ERVL- | chr2: 99216373-99216674 | + | 150 | 302 | COA5 | Proximal | 0 | Not documented | 1 |
| | Uniquely | | | | | MaLR | | | | | | | | | |
| MIR3_dup85367 | Multi | No | — | — | SINE | MIR | chr20: 1146922-1147038 | + | 160 | 117 | PSMF1 | Proximal | 0 | Not documented | 2 |
| Tigger1_dup11418 | Uniquely | No | — | — | DNA | DNA | chr20: 13430613-13432973 | − | 66 | 2361 | TASP1 | Proximal | 0 | Not documented | 3 |
| L1MB8_dup15815 | Multi/ | No | — | — | LINE | Other L1 | chr20: 23607651-23608723 | − | 90 | 1073 | CST3 | Proximal | 0 | Not documented | 4 |
| | Uniquely | | | | | | | | | | | | | | |
| MLT1K_dup17055 | Multi | No | — | — | LTR | ERVL- | chr20: 23609241-23609362 | − | 140 | 122 | CST3 | Proximal | 0 | Not documented | 5 |
| | | | | | | MaLR | | | | | | | | | |
| AluJr_dup72845 | Multi | No | — | — | SINE | Alu | chr20: 23609363-23609520 | − | 80 | 158 | CST3 | Proximal | 0 | Not documented | 6 |
| MLT1K_dup17056 | Multi | No | — | — | LTR | ERVL- | chr20: 23609521-23609907 | − | 140 | 387 | CST3 | Proximal | 0 | Not documented | 7 |
| | | | | | | MaLR | | | | | | | | | |
| L1MC4_dup27902 | Uniquely | No | — | — | LINE | Other L1 | chr20: 23617497-23617820 | + | 130 | 324 | CST3 | Proximal | 0 | Not documented | 8 |
| MER57A-int_{—} | Multi | No | — | — | LTR | ERV1 | chr20: 25277484-25277852 | + | 120 | 369 | PYGB | Proximal | 0 | Not documented | 9 |
| dup1845 | | | | | | | | | | | | | | | |
| MLT1A1_dup6438 | Multi | No | — | — | LTR | ERVL- | chr20: 25654805-25655216 | − | 89 | 412 | ZNF337 | Proximal | 0 | Not documented | 10 |
| | | | | | | MaLR | | | | | | | | | |
| LTR16B2_dup1486 | Multi | No | — | — | LTR | ERVL | chr20: 25655313-25655392 | − | 96 | 80 | ZNF337 | Proximal | 0 | Not documented | 11 |
| AluSx1_dup103716 | Multi | No | — | — | SINE | Alu | chr20: 25785050-25785335 | − | 62 | 286 | FAM182B | Proximal | 0 | Not documented | 12 |
| L1ME3F_dup4682 | Multi | No | — | — | LINE | Other L1 | chr20: 25785336-25785820 | − | 120 | 485 | FAM182B | Proximal | 0 | Not documented | 13 |
| SST1_dup554 | Multi/ | No | — | — | Other | Other | chr20: 25843554-25844836 | − | 130 | 1283 | FAM182B | Proximal | 0 | Not documented | 14 |
| | Uniquely | | | | | Repeats | | | | | | | | | |
| AluSx1_dup103740 | Multi | No | — | — | SINE | Alu | chr20: 26031843-26032109 | + | 65 | 267 | FAM182B | Distal | 182983 | Not documented | 15 |
| MER58A_dup12612 | Multi | No | — | — | DNA | DNA | chr20: 3028820-3029018 | + | 100 | 199 | MRPS26 | Proximal | 0 | Not documented | 16 |
| L2c_dup134679 | Multi | No | — | — | LINE | L2 | chr20: 30532762-30532796 | − | 52 | 35 | PDRG1 | Proximal | 0 | Not documented | 17 |
| L2a_dup162075 | Multi | No | — | — | LINE | L2 | chr20: 30824986-30825708 | − | 120 | 723 | POFUT1 | Proximal | 0 | Not documented | 18 |
| AluSx_dup134721 | Multi | No | — | — | SINE | Alu | chr20: 30921154-30921461 | + | 53 | 308 | KIF3B | Proximal | 0 | Not documented | 19 |
| AluSx1_dup103854 | Multi | No | — | — | SINE | Alu | chr20: 30987553-30987864 | − | 52 | 312 | ASXL1 | Proximal | 0 | Not documented | 20 |
| L1MEc_dup17917 | Multi | No | — | — | LINE | Other L1 | chr20: 30987865-30988771 | − | 94 | 907 | ASXL1 | Proximal | 0 | Not documented | 21 |
| AluSg_dup38881 | Multi | No | — | — | SINE | Alu | chr20: 31444485-31444787 | − | 50 | 303 | EFCAB8 | Proximal | 1943 | Not documented | 22 |
| Tigger11a_dup412 | Multi | No | — | — | DNA | DNA | chr20: 3204353-3204523 | − | 140 | 171 | ITPA | Proximal | 0 | Not documented | 23 |
| MIR3_dup86301 | Multi | No | — | — | SINE | MIR | chr20: 32245307-32245392 | − | 89 | 86 | NECAB3 | Proximal | 0 | Not documented | 24 |
| MIR_dup164657 | Multi/ | No | — | — | SINE | MIR | chr20: 329846-330012 | − | 160 | 167 | NRSN2 | Proximal | 0 | Not documented | 2925 |
| | Uniquely | | | | | | | | | | | | | | |
| AluJb_dup136343 | Multi | No | — | — | SINE | Alu | chr20: 33107223-33107307 | + | 80 | 85 | DYNLRB1 | Proximal | 0 | Not documented | 26 |
| Tigger15a_dup4938 | Uniquely | No | — | — | DNA | DNA | chr20: 33448952-33449071 | − | 130 | 120 | GGT7 | Proximal | 0 | Not documented | 27 |
| MIR3_dup85285 | Multi/ | No | — | — | SINE | MIR | chr20: 334566-334643 | + | 140 | 78 | NRSN2 | Proximal | 0 | Not documented | 28 |
| | Uniquely | | | | | | | | | | | | | | |
| MIRb_dup210983 | Multi/ | No | — | — | SINE | MIR | chr20: 334791-334965 | − | 130 | 175 | NRSN2 | Proximal | 0 | Not documented | 29 |
| | Uniquely | | | | | | | | | | | | | | |
| MER21B_dup2640 | Uniquely | No | — | — | LTR | ERVL | chr20: 33932497-33933319 | + | 70 | 823 | UQCC1 | Proximal | 0 | Not documented | 30 |
| MIRc_dup98551 | Multi | No | — | — | SINE | MIR | chr20: 35156915-35157046 | + | 73 | 132 | DLGAP4 | Proximal | 0 | Not documented | 31 |
| AluJb_dup136672 | Multi | No | — | — | SINE | Alu | chr20: 35380440-35380756 | − | 63 | 317 | DSN1 | Proximal | 0 | Not documented | 32 |
| MARNA_dup3249 | Multi | No | — | — | DNA | DNA | chr20: 35380828-35381076 | + | 120 | 249 | DSN1 | Proximal | 0 | Not documented | 33 |
| AluJb_dup136783 | Multi/ | No | — | — | SINE | Alu | chr20: 35927145-35927444 | − | 80 | 300 | MANBAL | Proximal | 0 | Not documented | 34 |
| | Uniquely | | | | | | | | | | | | | | |
| L2c_dup135115 | Multi | No | — | — | LINE | L2 | chr20: 36151850-36152015 | + | 120 | 166 | NNAT | Proximal | 0 | Not documented | 35 |
| AluSx_dup133742 | Multi | No | — | — | SINE | Alu | chr20: 3805298-3805594 | + | 54 | 297 | AP551 | Proximal | 0 | Not documented | 36 |
| L2b_dup93021 | Multi | No | — | — | LINE | L2 | chr20: 38214839-38214961 | + | 170 | 123 | DHX35 | Distal | 546474 | Not documented | 37 |
| MLT1J_dup14221 | Multi | No | — | — | LTR | ERVL- | chr20: 390955-391309 | − | 140 | 355 | RBCK1 | Proximal | 0 | Not documented | 38 |
| | | | | | | MaLR | | | | | | | | | |
| L1MD1_dup6464 | Multi | No | — | — | LINE | Other L1 | chr20: 3953877-3956204 | + | 77 | 2328 | RNF24 | Proximal | 0 | Not documented | 39 |
| L2b_dup93163 | Uniquely | No | — | — | LINE | L2 | chr20: 39799355-39799440 | − | 140 | 86 | PLCG1 | Proximal | 0 | Not documented | 40 |
| AluSx1_dup104476 | Uniquely | No | — | — | SINE | Alu | chr20: 39799945-39800249 | + | 30 | 305 | PLCG1 | Proximal | 0 | Not documented | 41 |
| AluSx_dup135659 | Multi/ | No | — | — | SINE | Alu | chr20: 39808272-39808569 | − | 29 | 298 | PLCG1 | Proximal | 0 | Not documented | 42 |
| | Uniquely | | | | | | | | | | | | | | |
| L1MA4A_dup6059 | Multi/ | No | — | — | LINE | Other L1 | chr20: 39817337-39818787 | − | 72 | 1451 | ZHX3 | Proximal | 0 | Not documented | 43 |
| | Uniquely | | | | | | | | | | | | | | |
| L1MB4_dup8872 | Multi/ | No | — | — | LINE | Other L1 | chr20: 39826936-39828342 | − | 110 | 1407 | ZHX3 | Proximal | 0 | Not documented | 44 |
| | Uniquely | | | | | | | | | | | | | | |
| L2a_dup162888 | Uniquely | No | — | — | LINE | L2 | chr20: 39896078-39896529 | − | 120 | 452 | ZHX3 | Proximal | 0 | Not documented | 45 |
| AluSz_dup93165 | Multi | No | — | — | SINE | Alu | chr20: 39987762-39988075 | − | 44 | 314 | LPIN3 | Proximal | 0 | Not documented | 46 |
| MIRc_dup99001 | Uniquely | No | — | — | SINE | MIR | chr20: 42853720-42853944 | + | 140 | 225 | OSER1 | Distal | 14290 | Not documented | 47 |
| MIR3_dup86988 | Uniquely | No | — | — | SINE | MIR | chr20: 43119011-43119092 | + | 130 | 82 | TTPAL | Proximal | 0 | Not documented | 48 |
| AluSg4_dup7082 | Uniquely | No | — | — | SINE | Alu | chr20: 43119438-43119740 | + | 40 | 303 | TTPAL | Proximal | 0 | Not documented | 49 |
| MER3_dup10511 | Multi | No | — | — | DNA | DNA | chr20: 44054599-44054792 | − | 99 | 194 | PIGT | Proximal | 0 | Not documented | 2950 |
| FLAM_A_dup15517 | Uniquely | No | — | — | SINE | Alu | chr20: 44470649-44470778 | − | 80 | 130 | SNX21 | Proximal | 0 | Not documented | 51 |
| LTR16D2_dup258 | Uniquely | No | — | — | LTR | ERVL | chr20: 45137921-45138455 | − | 120 | 535 | ZNF334 | Proximal | 0 | Not documented | 52 |
| L2_dup54492 | Uniquely | No | — | — | LINE | L2 | chr20: 45138693-45139082 | − | 170 | 390 | ZNF334 | Proximal | 0 | Not documented | 53 |
| MER63C_dup863 | Uniquely | No | — | — | DNA | DNA | chr20: 45363955-45364874 | + | 97 | 920 | SLC2A10 | Proximal | 0 | Not documented | 54 |
| AluJb_dup137564 | Multi/ | No | — | — | SINE | Alu | chr20: 48251115-48251419 | + | 58 | 305 | B4GALT5 | Proximal | 0 | Not documented | 55 |
| | Uniquely | | | | | | | | | | | | | | |
| AluY_dup113714 | Multi | No | — | — | SINE | Alu | chr20: 49367881-49368180 | − | 29 | 300 | PARD6B | Proximal | 0 | Not documented | 56 |
| AluJb_dup137727 | Uniquely | No | — | — | SINE | Alu | chr20: 49557644-49557756 | + | 52 | 113 | DPM1 | Proximal | 0 | Not documented | 57 |
| MLT1J_dup14649 | Uniquely | No | — | — | LTR | ERVL- | chr20: 50035762-50036195 | + | 120 | 434 | NFATC2 | Proximal | 0 | Not documented | 58 |
| | | | | | | MaLR | | | | | | | | | |
| L2b_dup93871 | Uniquely | No | — | — | LINE | L2 | chr20: 50059521-50059921 | + | 150 | 401 | NFATC2 | Proximal | 0 | Not documented | 59 |
| AluJo_dup68933 | Uniquely | No | — | — | SINE | Alu | chr20: 50156012-50156337 | − | 78 | 326 | NFATC2 | Proximal | 0 | Not documented | 60 |
| AluY_dup113746 | Multi | No | — | — | SINE | Alu | chr20: 50160515-50160825 | − | 26 | 311 | NFATC2 | Proximal | 0 | Not documented | 61 |
| MER5B_dup24162 | Uniquely | No | — | — | DNA | DNA | chr20: 50161194-50161376 | + | 83 | 183 | NFATC2 | Proximal | 0 | Not documented | 62 |
| MLT1C_dup18739 | Multi | No | — | — | LTR | ERVL- | chr20: 50172412-50172870 | + | 120 | 459 | NFATC2 | Proximal | 0 | Not documented | 63 |
| | | | | | | MaLR | | | | | | | | | |
| L2a_dup164290 | Multi/ | No | — | — | LINE | L2 | chr20: 50172871-50173111 | + | 150 | 241 | NFATC2 | Proximal | 0 | Not documented | 64 |
| | Uniquely | | | | | | | | | | | | | | |
| MIRb_dup216176 | Uniquely | No | — | — | SINE | MIR | chr20: 50173679-50173817 | + | 130 | 139 | NFATC2 | Proximal | 0 | Not documented | 65 |
| MIR_dup168800 | Multi/ | No | — | — | SINE | MIR | chr20: 50176039-50176231 | + | 130 | 193 | NFATC2 | Proximal | 0 | Not documented | 66 |
| | Uniquely | | | | | | | | | | | | | | |
| MIR3_dup87417 | Multi | No | — | — | SINE | MIR | chr20: 50176253-50176354 | + | 130 | 102 | NFATC2 | Proximal | 0 | Not documented | 67 |
| MIRb_dup216179 | Multi | No | — | — | SINE | MIR | chr20: 50176355-50176491 | − | 140 | 137 | NFATC2 | Proximal | 0 | Not documented | 68 |
| MIR_dup168801 | Multi/ | No | — | — | SINE | MIR | chr20: 50176661-50176873 | − | 120 | 213 | NFATC2 | Proximal | 0 | Not documented | 69 |
| | Uniquely | | | | | | | | | | | | | | |
| AluSz_dup94008 | Multi | No | — | — | SINE | Alu | chr20: 54942413-54942704 | + | 44 | 292 | FAM210B | Proximal | 0 | Not documented | 70 |
| AluSx1_dup105421 | Multi | No | — | — | SINE | Alu | chr20: 56940716-56941003 | − | 28 | 288 | RAB22A | Proximal | 0 | Not documented | 71 |
| L1MA4_dup9731 | Multi | No | — | — | LINE | Other L1 | chr20: 56941386-56941680 | + | 78 | 295 | RAB22A | Proximal | 0 | Not documented | 72 |
| L1MD3_dup5024 | Multi | No | — | — | LINE | Other L1 | chr20: 627273-627575 | − | 65 | 303 | SRXN1 | Proximal | 0 | Not documented | 73 |
| MLT1J_dup14225 | Multi | No | — | — | LTR | ERVL- | chr20: 628762-629018 | + | 150 | 257 | SRXN1 | Proximal | 0 | Not documented | 74 |
| | | | | | | MaLR | | | | | | | | | |
| AluY_dup112189 | Multi | No | — | — | SINE | Alu | chr20: 8397722-8397937 | − | 31 | 216 | PLCB1 | Proximal | 0 | Not documented | 2975 |
| L2c_dup134056 | Multi | No | — | — | LINE | L2 | chr20: 8398183-8398267 | − | 160 | 85 | PLCB1 | Proximal | 0 | Not documented | 76 |
| L1MB1_dup6015 | Multi | No | — | — | LINE | Other L1 | chr21: 10003065-10003684 | + | 68 | 620 | TPTE | Distal | 902518 | Not documented | 77 |
| L1MEf_dup13522 | Multi | No | — | — | LINE | Other L1 | chr21: 10005329-10006715 | − | 140 | 1387 | TPTE | Distal | 899487 | Not documented | 78 |
| L1PA4_dup11589 | Uniquely | No | — | — | LINE | L1PA\|B\|x | chr21: 10021922-10028052 | − | 26 | 6131 | TPTE | Distal | 878150 | EVE annotated | 79 |
| L2b_dup94566 | Multi | No | — | — | LINE | L2 | chr21: 10118613-10118822 | − | 160 | 210 | TPTE | Distal | 787380 | Not documented | 80 |
| LTR16A_dup6750 | Multi | No | — | — | LTR | ERVL | chr21: 10132838-10133236 | + | 140 | 399 | TPTE | Distal | 772966 | Not documented | 81 |
| AluSx1_dup105631 | Multi | No | — | — | SINE | Alu | chr21: 10191880-10192196 | + | 49 | 317 | TPTE | Distal | 714006 | Not documented | 82 |
| L1MCa_dup7316 | Multi | No | — | — | LINE | Other L1 | chr21: 10197517-10198958 | − | 85 | 1442 | TPTE | Distal | 707244 | Not documented | 83 |
| CER_dup39 | Multi | No | — | — | Other | Other | chr21: 10199089-10200505 | − | 49 | 1417 | TPTE | Distal | 705697 | Not documented | 84 |
| | | | | | | Repeats | | | | | | | | | |
| Tigger1_dup11625 | Multi | No | — | — | DNA | DNA | chr21: 10366923-10368944 | − | 70 | 2022 | TPTE | Distal | 537258 | Not documented | 85 |
| L1MA6_dup5287 | Multi | No | — | — | LINE | Other L1 | chr21: 10417616-10418219 | + | 89 | 604 | TPTE | Distal | 487983 | Not documented | 86 |
| SST1_dup588 | Multi | No | — | — | Other | Other | chr21: 10597119-10598373 | − | 32 | 1255 | TPTE | Distal | 307829 | Not documented | 87 |
| | | | | | | Repeats | | | | | | | | | |
| L1MDa_dup6824 | Multi | No | — | — | LINE | Other L1 | chr21: 11071057-11072231 | + | 120 | 1175 | TPTE | Distal | 41339 | Not documented | 88 |
| MLT1A0_dup19886 | Uniquely | No | — | — | LTR | ERVL- | chr21: 17618821-17619157 | − | 92 | 337 | USP25 | Distal | 366445 | Not documented | 89 |
| | | | | | | MaLR | | | | | | | | | |
| AluSz_dup94326 | Uniquely | No | — | — | SINE | Alu | chr21: 17647423-17647722 | + | 65 | 300 | USP25 | Distal | 395047 | Not documented | 90 |
| LTR78_dup4636 | Uniquely | No | — | — | LTR | ERV1 | chr21: 17670471-17671157 | − | 160 | 687 | USP25 | Distal | 418095 | Not documented | 91 |
| L2a_dup165638 | Uniquely | No | — | — | LINE | L2 | chr21: 17692085-17692538 | − | 150 | 454 | USP25 | Distal | 439709 | Not documented | 92 |
| L4_dup16342 | Multi | No | — | — | LINE | RTE | chr21: 17791734-17791794 | + | 140 | 61 | USP25 | Distal | 539358 | Not documented | 93 |
| L1MA1_dup4138 | Uniquely | No | — | — | LINE | Other L1 | chr21: 17849534-17852261 | − | 47 | 2728 | USP25 | Distal | 597158 | Not documented | 94 |
| L1PA16_dup13687 | Uniquely | No | — | — | LINE | L1PA\|B\|x | chr21: 17886197-17888439 | − | 56 | 2243 | USP25 | Distal | 633821 | Not documented | 95 |
| L1HS_dup1510 | Multi | No | — | — | LINE | L1PA\|B\|x | chr21: 17916705-17917238 | − | 2.7 | 534 | USP25 | Distal | 664329 | Not documented | 96 |
| AluSx1_dup105740 | Uniquely | No | — | — | SINE | Alu | chr21: 17918362-17918656 | + | 51 | 295 | USP25 | Distal | 665986 | Not documented | 97 |
| AluSz_dup94331 | Uniquely | No | — | — | SINE | Alu | chr21: 17930581-17930884 | + | 72 | 304 | USP25 | Distal | 678205 | Not documented | 98 |
| L2c_dup136709 | Multi/ | No | — | — | LINE | L2 | chr21: 17979512-17979578 | + | 89 | 67 | USP25 | Distal | 727136 | Not documented | 99 |
| | Uniquely | | | | | | | | | | | | | | |
| L2a_dup165651 | Multi/ | No | — | — | LINE | L2 | chr21: 17979709-17980366 | + | 130 | 658 | USP25 | Distal | 727333 | Not documented | 3000 |
| | Uniquely | | | | | | | | | | | | | | |
| MER77B_dup1222 | Uniquely | No | — | — | LTR | ERVL | chr21: 17989633-17989918 | − | 100 | 286 | USP25 | Distal | 737257 | Not documented | 1 |
| AluY_dup114501 | Multi | No | — | — | SINE | Alu | chr21: 17992222-17992488 | − | 19 | 267 | USP25 | Distal | 739846 | Not documented | 2 |
| L1MD2_dup10682 | Multi/ | No | — | — | LINE | Other L1 | chr21: 17992725-17993500 | − | 87 | 776 | USP25 | Distal | 740349 | Not documented | 3 |
| | Uniquely | | | | | | | | | | | | | | |
| MER33_dup9200 | Multi | No | — | — | DNA | DNA | chr21: 17993501-17993557 | + | 65 | 57 | USP25 | Distal | 741125 | Not documented | 4 |
| MIR_dup170119 | Multi/ | No | — | — | SINE | MIR | chr21: 17993752-17993953 | + | 130 | 202 | USP25 | Distal | 741376 | Not documented | 5 |
| | Uniquely | | | | | | | | | | | | | | |
| L2b_dup94640 | Multi | No | — | — | LINE | L2 | chr21: 17993967-17994061 | + | 150 | 95 | USP25 | Distal | 741591 | Not documented | 6 |
| THE1B_dup21743 | Multi/ | No | — | — | LTR | ERVL- | chr21: 17994062-17994430 | − | 51 | 369 | USP25 | Distal | 741686 | Not documented | 7 |
| | Uniquely | | | | | MaLR | | | | | | | | | |
| L2b_dup94641 | Multi | No | — | — | LINE | L2 | chr21: 17994431-17994554 | + | 150 | 124 | USP25 | Distal | 742055 | Not documented | 8 |
| AluSx1_dup105819 | Uniquely | No | — | — | SINE | Alu | chr21: 22440445-22440745 | − | 48 | 301 | NCAM2 | Proximal | 0 | Not documented | 9 |
| AluSg_dup39585 | Multi | No | — | — | SINE | Alu | chr21: 22440777-22441044 | − | 47 | 268 | NCAM2 | Proximal | 0 | Not documented | 10 |
| MER50_dup2468 | Uniquely | No | — | — | LTR | ERV1 | chr21: 22453103-22453802 | − | 64 | 700 | NCAM2 | Proximal | 0 | Not documented | 11 |
| AluSz_dup94464 | Multi | No | — | — | SINE | Alu | chr21: 27088291-27088606 | − | 49 | 316 | JAM2 | Proximal | 0 | Not documented | 12 |
| MER58A_dup13146 | Multi/ | No | — | — | DNA | DNA | chr21: 27088853-27089067 | − | 88 | 215 | ATP5J | Proximal | 0 | Not documented | 13 |
| | Uniquely | | | | | | | | | | | | | | |
| MIR_dup170447 | Multi | No | — | — | SINE | MIR | chr21: 27099729-27099915 | − | 110 | 187 | ATP5J | Proximal | 0 | Not documented | 14 |
| L1M5_dup62402 | Multi | No | — | — | LINE | Other L1 | chr21: 27518678-27518838 | − | 100 | 161 | APP | Proximal | 0 | Not documented | 15 |
| AluSz_dup94514 | Uniquely | No | — | — | SINE | Alu | chr21: 28213525-28213821 | + | 46 | 297 | ADAMTS1 | Proximal | 0 | Not documented | 16 |
| AluJb_dup138937 | Multi/ | No | — | — | SINE | Alu | chr21: 33641107-33641288 | − | 80 | 182 | MIS18A | Proximal | 0 | Not documented | 17 |
| | Uniquely | | | | | | | | | | | | | | |
| MIRc_dup100306 | Multi/ | No | — | — | SINE | MIR | chr21: 33766064-33766172 | + | 160 | 109 | C21orf119 | Proximal | 0 | Not documented | 18 |
| | Uniquely | | | | | | | | | | | | | | |
| L1P4a_dup556 | Uniquely | No | — | — | LINE | L1PA\|B\|x | chr21: 33936552-33939507 | − | 83 | 2956 | TCP10L | Distal | 9356 | Not documented | 19 |
| MER102c_dup3418 | Multi | No | — | — | DNA | DNA | chr21: 34163224-34163396 | + | 110 | 173 | C21orf62 | Proximal | 0 | Not documented | 20 |
| AluSp_dup47788 | Multi/ | No | — | — | SINE | Alu | chr21: 34164205-34164476 | − | 33 | 272 | C21orf62 | Proximal | 0 | Not documented | 21 |
| | Uniquely | | | | | | | | | | | | | | |
| MamRep1161_{—} | Multi | No | — | — | DNA | DNA | chr21: 34164653-34164777 | + | 140 | 125 | C21orf62 | Proximal | 0 | Not documented | 22 |
| dup1719 | | | | | | | | | | | | | | | |
| AluSp_dup47789 | Multi | No | — | — | SINE | Alu | chr21: 34165213-34165290 | − | 41 | 78 | C21orf62 | Proximal | 0 | Not documented | 23 |
| AluSx_dup137854 | Multi/ | No | — | — | SINE | Alu | chr21: 34165291-34165567 | − | 33 | 277 | C21orf62 | Proximal | 0 | Not documented | 24 |
| | Uniquely | | | | | | | | | | | | | | |
| L2_dup55197 | Multi/ | No | — | — | LINE | L2 | chr21: 34182871-34183127 | + | 140 | 257 | C21orf62 | Proximal | 0 | Not documented | 3025 |
| | Uniquely | | | | | | | | | | | | | | |
| FAM_dup4685 | Multi | No | — | — | SINE | Alu | chr21: 34634846-34635011 | − | 90 | 166 | IFNAR2 | Proximal | 0 | Not documented | 26 |
| AluSx1_dup106216 | Multi | No | — | — | SINE | Alu | chr21: 34822512-34822802 | − | 53 | 291 | TMEM50B | Proximal | 0 | Not documented | 27 |
| AluJb_dup139037 | Multi | No | — | — | SINE | Alu | chr21: 34896575-34896785 | + | 65 | 211 | GART | Proximal | 0 | Not documented | 28 |
| MARNA_dup3295 | Uniquely | No | — | — | DNA | DNA | chr21: 34967492-34967673 | + | 100 | 182 | CRYZL1 | Proximal | 0 | Not documented | 29 |
| MIR_dup170855 | Uniquely | No | — | — | SINE | MIR | chr21: 34967746-34967930 | + | 110 | 185 | CRYZL1 | Proximal | 0 | Not documented | 30 |
| AluSx_dup137979 | Multi | No | — | — | SINE | Alu | chr21: 35761084-35761398 | + | 48 | 315 | SMIM11 | Proximal | 0 | Not documented | 31 |
| Charlie15a_{—} | Multi/ | No | — | — | DNA | DNA | chr21: 35774676-35774833 | − | 140 | 158 | SMIM11 | Proximal | 0 | Not documented | 32 |
| dup3358 | Uniquely | | | | | | | | | | | | | | |
| AluSc8_dup20928 | Uniquely | No | — | — | SINE | Alu | chr21: 35891688-35891994 | − | 40 | 307 | RCAN1 | Proximal | 0 | Not documented | 33 |
| AluJb_dup139306 | Multi | No | — | — | SINE | Alu | chr21: 38596552-38596814 | + | 62 | 263 | DSCR3 | Proximal | 0 | Not documented | 34 |
| AluSg4_dup7204 | Multi | No | — | — | SINE | Alu | chr21: 38597005-38597292 | + | 40 | 288 | DSCR3 | Proximal | 0 | Not documented | 35 |
| MIRc_dup100515 | Multi/ | No | — | — | SINE | MIR | chr21: 40668486-40668703 | − | 95 | 218 | BRWD1 | Proximal | 0 | Not documented | 36 |
| | Uniquely | | | | | | | | | | | | | | |
| Charlie2b_dup3510 | Multi | No | — | — | DNA | DNA | chr21: 42801857-42802642 | + | 140 | 786 | MX1 | Proximal | 0 | Not documented | 37 |
| L1MB5_dup9525 | Multi/ | No | — | — | LINE | Other L1 | chr21: 44473012-44473559 | + | 92 | 548 | CBS | Proximal | 0 | Not documented | 38 |
| | Uniquely | | | | | | | | | | | | | | |
| L1MC3_dup13070 | Multi | No | — | — | LINE | Other L1 | chr21: 44493417-44493569 | + | 84 | 153 | CBS | Proximal | 0 | Not documented | 39 |
| AluSc8_dup21002 | Multi | No | — | — | SINE | Alu | chr21: 46190335-46190631 | − | 54 | 297 | UBE2G2 | Proximal | 0 | Not documented | 40 |
| L1M5_dup62832 | Multi/ | No | — | — | LINE | Other L1 | chr21: 46715858-46716712 | − | 150 | 855 | POFUT2 | Distal | 8046 | Not documented | 41 |
| | Uniquely | | | | | | | | | | | | | | |
| AluSg_dup39947 | Multi | No | — | — | SINE | Alu | chr21: 48018268-48018527 | − | 31 | 260 | S100B | Proximal | 349 | Not documented | 42 |
| L1M2_dup8885 | Multi/ | No | — | — | LINE | Other L1 | chr21: 48019159-48019208 | + | 110 | 50 | S100B | Proximal | 0 | Not documented | 43 |
| | Uniquely | | | | | | | | | | | | | | |
| AluJb_dup139741 | Multi/ | No | — | — | SINE | Alu | chr21: 48020173-48020480 | + | 66 | 308 | S100B | Proximal | 0 | Not documented | 44 |
| | Uniquely | | | | | | | | | | | | | | |
| L1M5_dup62907 | Multi | No | — | — | LINE | Other L1 | chr21: 48079369-48079509 | + | 130 | 141 | PRMT2 | Proximal | 0 | Not documented | 45 |
| L2a_dup167023 | Multi | No | — | — | LINE | L2 | chr21: 48084816-48084859 | + | 93 | 44 | PRMT2 | Proximal | 0 | Not documented | 46 |
| MER31-int_dup1977 | Multi | No | — | — | LTR | ERV1 | chr21: 48111224-48111670 | − | 98 | 447 | PRMT2 | Distal | 26189 | Not documented | 47 |
| L1M2_dup8781 | Multi | No | — | — | LINE | Other L1 | chr21: 9554486-9555191 | − | 91 | 706 | TPTE | Distal | 1351011 | Not documented | 48 |
| HERVL-int_dup2184 | Multi | No | — | — | LTR | ERVL | chr21: 9592473-9594904 | − | 54 | 2432 | TPTE | Distal | 1311298 | Not documented | 49 |
| AluSx_dup137177 | Multi | No | — | — | SINE | Alu | chr21: 9676211-9676504 | + | 62 | 294 | TPTE | Distal | 1229698 | Not documented | 3050 |
| L1MCa_dup7311 | Multi | No | — | — | LINE | Other L1 | chr21: 9681337-9682785 | − | 85 | 1449 | TPTE | Distal | 1223417 | Not documented | 51 |
| GSATII_dup173 | Multi | No | — | — | Other | Other | chr21: 9702682-9704227 | − | 59 | 1546 | TPTE | Distal | 1201975 | Not documented | 52 |
| | | | | | | Repeats | | | | | | | | | |
| L1M4_dup17297 | Multi | No | — | — | LINE | Other L1 | chr21: 9709237-9711653 | − | 94 | 2417 | TPTE | Distal | 1194549 | Not documented | 53 |
| L1PA10_dup6916 | Multi | No | — | — | LINE | L1PA\|B\|x | chr21: 9827579-9827850 | + | 72 | 272 | TPTE | Distal | 1078352 | Not documented | 54 |
| MER21B_dup2666 | Multi | No | — | — | LTR | ERVL | chr21: 9828046-9828304 | − | 130 | 259 | TPTE | Distal | 1077898 | Not documented | 55 |
| MER5A_dup33783 | Multi | No | — | — | DNA | DNA | chr21: 9828723-9828790 | − | 130 | 68 | TPTE | Distal | 1077412 | Not documented | 56 |
| L3_dup43801 | Multi | No | — | — | LINE | CR1 | chr21: 9837789-9838289 | + | 160 | 501 | TPTE | Distal | 1067913 | Not documented | 57 |
| MER65C_dup657 | Multi | No | — | — | LTR | ERV1 | chr21: 9848875-9849338 | − | 96 | 464 | TPTE | Distal | 1056864 | Not documented | 58 |
| MER61F_dup149 | Multi | No | — | — | LTR | ERV1 | chr21: 9862959-9863547 | − | 59 | 589 | TPTE | Distal | 1042655 | Not documented | 59 |
| GSATII_dup175 | Multi | No | — | — | Other | Other | chr21: 9880361-9881894 | − | 57 | 1534 | TPTE | Distal | 1024308 | Not documented | 60 |
| | | | | | | Repeats | | | | | | | | | |
| AluSp_dup47509 | Multi | No | — | — | SINE | Alu | chr21: 9883749-9883916 | − | 73 | 168 | TPTE | Distal | 1022286 | Not documented | 61 |
| L1PB_dup1708 | Multi | No | — | — | LINE | L1PA\|B\|x | chr21: 9883917-9885571 | − | 45 | 1655 | TPTE | Distal | 1020631 | Not documented | 62 |
| MLT2B3_dup3182 | Multi | No | — | — | LTR | ERVL | chr21: 9887977-9888372 | + | 90 | 396 | TPTE | Distal | 1017830 | Not documented | 63 |
| Tigger4_dup1397 | Multi | No | — | — | DNA | DNA | chr21: 9896052-9896524 | + | 79 | 473 | TPTE | Distal | 1009678 | Not documented | 64 |
| AluSz_dup94239 | Multi | No | — | — | SINE | Alu | chr21: 9927681-9927977 | − | 57 | 297 | TPTE | Distal | 978225 | Not documented | 65 |
| MSTA-int_dup2997 | Multi | No | — | — | LTR | ERVL- | chr21: 9933455-9933706 | + | 71 | 252 | TPTE | Distal | 972496 | Not documented | 66 |
| | | | | | | MaLR | | | | | | | | | |
| CER_dup38 | Multi | No | — | — | Other | Other | chr21: 9935221-9939398 | + | 46 | 4178 | TPTE | Distal | 966804 | Not documented | 67 |
| | | | | | | Repeats | | | | | | | | | |
| AluSx_dup137188 | Multi | No | — | — | SINE | Alu | chr21: 9948752-9949052 | + | 60 | 301 | TPTE | Distal | 957150 | Not documented | 68 |
| L1MB3_dup16599 | Multi | No | — | — | LINE | Other L1 | chr21: 9951150-9951589 | + | 92 | 440 | TPTE | Distal | 954613 | Not documented | 69 |
| MER52A_dup1751 | Multi | No | — | — | LTR | ERV1 | chr21: 9957780-9958988 | + | 90 | 1209 | TPTE | Distal | 947214 | Not documented | 70 |
| MER57-int_dup1171 | Multi | No | — | — | LTR | ERV1 | chr21: 9958989-9959905 | + | 59 | 917 | TPTE | Distal | 946297 | Not documented | 71 |
| MER57-int_dup1174 | Multi | No | — | — | LTR | ERV1 | chr21: 9962008-9962960 | + | 55 | 953 | TPTE | Distal | 943242 | Not documented | 72 |
| MER57A1_dup1007 | Multi | No | — | — | LTR | ERV1 | chr21: 9964332-9964730 | + | 70 | 399 | TPTE | Distal | 941472 | Not documented | 73 |
| MER66A_dup304 | Multi | No | — | — | LTR | ERV1 | chr21: 9969796-9970174 | + | 100 | 379 | TPTE | Distal | 936028 | Not documented | 74 |
| AluSq_dup20797 | Multi | No | — | — | SINE | Alu | chr21: 9985587-9985875 | − | 49 | 289 | TPTE | Distal | 920327 | Not documented | 3075 |
| LTR49-int_dup2049 | Multi | No | — | — | LTR | ERV1 | chr21: 9986238-9986713 | − | 75 | 476 | TPTE | Distal | 919489 | Not documented | 76 |
| LTR49-int_dup2053 | Multi | No | — | — | LTR | ERV1 | chr21: 9988145-9988248 | − | 130 | 104 | TPTE | Distal | 917954 | Not documented | 77 |
| MER9a2_dup297 | Multi | No | — | — | LTR | ERVK | chr21: 9992671-9993127 | − | 29 | 457 | TPTE | Distal | 913075 | Not documented | 78 |
| HERVK14-int_{—} | Multi | No | — | — | LTR | ERVK | chr21: 9993128-9994811 | − | 38 | 1684 | TPTE | Distal | 911391 | Not documented | 79 |
| dup359 | | | | | | | | | | | | | | | |
| HERVK14-int_{—} | Multi | No | — | — | LTR | ERVK | chr21: 9994806-9994906 | − | 50 | 101 | TPTE | Distal | 911296 | Not documented | 80 |
| dup360 | | | | | | | | | | | | | | | |
| AluJb_dup139875 | Uniquely | No | — | — | SINE | Alu | chr22: 18060749-18061044 | − | 60 | 296 | SLC25A18 | Proximal | 0 | Not documented | 81 |
| MIRb_dup219210 | Multi | No | — | — | SINE | MIR | chr22: 18062477-18062610 | − | 120 | 134 | SLC25A18 | Proximal | 0 | Not documented | 82 |
| AluSx_dup139123 | Multi | No | — | — | SINE | Alu | chr22: 21304678-21305001 | − | 45 | 324 | CRKL | Proximal | 0 | Not documented | 83 |
| TAR1_dup153 | Multi | No | — | — | Other | Other | chr22: 22652106-22652554 | − | 75 | 449 | VPREB1 | Distal | 52180 | Not documented | 84 |
| | | | | | | Repeats | | | | | | | | | |
| AluSc8_dup21135 | Multi | No | — | — | SINE | Alu | chr22: 22674449-22674740 | − | 48 | 292 | VPREB1 | Distal | 74523 | Not documented | 85 |
| AluSz_dup95209 | Multi | No | — | — | SINE | Alu | chr22: 22675348-22675462 | − | 59 | 115 | VPREB1 | Distal | 75422 | Not documented | 86 |
| MLT1H2_dup4614 | Uniquely | No | — | — | LTR | ERVL- | chr22: 26584189-26584502 | − | 110 | 314 | SEZ6L | Proximal | 0 | Not documented | 87 |
| | | | | | | MaLR | | | | | | | | | |
| HAL1b_dup4768 | Multi | No | — | — | LINE | Other L1 | chr22: 26956921-26957126 | + | 140 | 206 | TPST2 | Proximal | 0 | Not documented | 88 |
| L1MA2_dup7452 | Uniquely | No | — | — | LINE | Other L1 | chr22: 28552774-28555361 | + | 39 | 2588 | TTC28 | Proximal | 0 | Not documented | 89 |
| AluSq2_dup53610 | Multi | No | — | — | SINE | Alu | chr22: 28962923-28963227 | + | 48 | 305 | TTC28 | Proximal | 0 | Not documented | 90 |
| AluSg_dup40219 | Multi | No | — | — | SINE | Alu | chr22: 29153194-29153498 | + | 57 | 305 | HSCB | Proximal | 0 | Not documented | 91 |
| AluSp_dup48381 | Uniquely | No | — | — | SINE | Alu | chr22: 29452095-29452388 | − | 42 | 294 | ZNRF3 | Proximal | 0 | Not documented | 92 |
| MIRc_dup101304 | Multi | No | — | — | SINE | MIR | chr22: 29663345-29663445 | + | 140 | 101 | RHBDD3 | Proximal | 0 | Not documented | 93 |
| MER30_dup4068 | Multi | No | — | — | DNA | DNA | chr22: 30166250-30166470 | − | 54 | 221 | UQCR10 | Proximal | 0 | Not documented | 94 |
| MER11C_dup841 | Multi/ | No | — | — | LTR | ERVK | chr22: 36586041-36587115 | − | 32 | 1075 | APOL4 | Proximal | 0 | Not documented | 95 |
| | Uniquely | | | | | | | | | | | | | | |
| AluY_dup117463 | Multi | No | — | — | SINE | Alu | chr22: 36623080-36623387 | + | 30 | 308 | APOL2 | Proximal | 0 | Not documented | 96 |
| LTR10A_dup305 | Multi | No | — | — | LTR | ERV1 | chr22: 36883200-36883685 | − | 46 | 486 | FOXRED2 | Proximal | 0 | Not documented | 97 |
| MLT1D_dup20378 | Multi | No | — | — | LTR | ERVL- | chr22: 38226818-38227278 | − | 110 | 461 | ANKRD54 | Proximal | 0 | Not documented | 98 |
| | | | | | | MaLR | | | | | | | | | |
| MIR3_dup89858 | Multi | No | — | — | SINE | MIR | chr22: 42070584-42070658 | − | 90 | 75 | NHP2L1 | Proximal | 0 | Not documented | 99 |
| L1MC4a_dup27276 | Multi | No | — | — | LINE | Other L1 | chr22: 44888477-44889045 | + | 130 | 569 | LDOC1L | Proximal | 0 | Not documented | 3100 |
| AluSc5_dup6774 | Multi | No | — | — | SINE | Alu | chr22: 51206401-51206690 | + | 27 | 290 | RABL2B | Proximal | 0 | Not documented | 1 |
| MER31-int_dup2040 | Multi | No | — | — | LTR | ERV1 | chr22: 51237636-51238082 | − | 97 | 447 | RABL2B | Distal | 15546 | Not documented | 2 |
| MER68_dup275 | Multi | No | — | — | LTR | ERVL | chr3: 111711727-111712276 | + | 79 | 550 | ABHD10 | Proximal | 0 | Not documented | 3 |
| MIRb_dup40720 | Uniquely | No | — | — | SINE | MIR | chr3: 11207605-11207714 | − | 140 | 110 | HRH1 | Proximal | 0 | Not documented | 4 |
| MIR3_dup19606 | Multi | No | — | — | SINE | MIR | chr3: 112302735-112302799 | − | 110 | 65 | SLC35A5 | Proximal | 0 | Not documented | 5 |
| MARNA_dup606 | Multi | No | — | — | DNA | DNA | chr3: 11302659-11302950 | + | 140 | 292 | HRH1 | Proximal | 0 | Not documented | 6 |
| MIRb_dup40741 | Multi | No | — | — | SINE | MIR | chr3: 11303022-11303182 | − | 130 | 161 | HRH1 | Proximal | 0 | Not documented | 7 |
| AluSz6_dup7422 | Multi | No | — | — | SINE | Alu | chr3: 11303241-11303533 | + | 59 | 293 | HRH1 | Proximal | 0 | Not documented | 8 |
| MARNA_dup607 | Multi | No | — | — | DNA | DNA | chr3: 11303667-11303761 | + | 130 | 95 | HRH1 | Proximal | 0 | Not documented | 9 |
| L1M1_dup1649 | Uniquely | No | — | — | LINE | Other L1 | chr3: 113402614-113405304 | − | 74 | 2691 | KIAA2018 | Proximal | 0 | Not documented | 10 |
| LTR12_dup121 | Uniquely | No | — | — | LTR | ERV1 | chr3: 11389721-11390760 | − | 39 | 1040 | ATG7 | Proximal | 0 | Not documented | 11 |
| L4_dup3360 | Multi | No | — | — | LINE | RTE | chr3: 114045078-114045117 | − | 80 | 40 | ZBTB20 | Distal | 11825 | Not documented | 12 |
| MIRc_dup22808 | Multi | No | — | — | SINE | MIR | chr3: 114046679-114046739 | + | 130 | 61 | ZBTB20 | Distal | 10203 | Not documented | 13 |
| MIR3_dup19655 | Multi/ | No | — | — | SINE | MIR | chr3: 114099650-114099848 | + | 80 | 199 | ZBTB20 | Proximal | 0 | Not documented | 14 |
| | Uniquely | | | | | | | | | | | | | | |
| MIR3_dup19658 | Uniquely | No | — | — | SINE | MIR | chr3: 114122699-114122841 | + | 130 | 143 | ZBTB20 | Proximal | 0 | Not documented | 15 |
| MIRc_dup22818 | Multi | No | — | — | SINE | MIR | chr3: 114124480-114124538 | − | 120 | 59 | ZBTB20 | Proximal | 0 | Not documented | 16 |
| L2c_dup31601 | Multi/ | No | — | — | LINE | L2 | chr3: 114185810-114185954 | − | 190 | 145 | ZBTB20 | Proximal | 0 | Not documented | 17 |
| | Uniquely | | | | | | | | | | | | | | |
| L2a_dup35753 | Multi/ | No | — | — | LINE | L2 | chr3: 114186679-114186738 | − | 130 | 60 | ZBTB20 | Proximal | 0 | Not documented | 18 |
| | Uniquely | | | | | | | | | | | | | | |
| L1M5_dup12548 | Multi | No | — | — | LINE | Other L1 | chr3: 114187393-114187558 | − | 120 | 166 | ZBTB20 | Proximal | 0 | Not documented | 19 |
| MER102c_dup726 | Multi | No | — | — | DNA | DNA | chr3: 114187655-114188023 | − | 110 | 369 | ZBTB20 | Proximal | 0 | Not documented | 20 |
| AluY_dup22854 | Uniquely | No | — | — | SINE | Alu | chr3: 114268786-114269084 | − | 45 | 299 | ZBTB20 | Proximal | 0 | Not documented | 21 |
| Tigger1_dup2474 | Uniquely | No | — | — | DNA | DNA | chr3: 114281307-114281993 | − | 60 | 687 | ZBTB20 | Proximal | 0 | Not documented | 22 |
| AluSp_dup9488 | Multi/ | No | — | — | SINE | Alu | chr3: 114312220-114312530 | − | 42 | 311 | ZBTB20 | Proximal | 0 | Not documented | 23 |
| | Uniquely | | | | | | | | | | | | | | |
| AluSc8_dup4131 | Multi/ | No | — | — | SINE | Alu | chr3: 114313111-114313373 | − | 43 | 263 | ZBTB20 | Proximal | 0 | Not documented | 24 |
| | Uniquely | | | | | | | | | | | | | | |
| AluJr_dup14125 | Uniquely | No | — | — | SINE | Alu | chr3: 114337086-114337404 | − | 74 | 319 | ZBTB20 | Proximal | 0 | Not documented | 3125 |
| AluSp_dup9490 | Multi | No | — | — | SINE | Alu | chr3: 114339130-114339437 | − | 47 | 308 | ZBTB20 | Proximal | 0 | Not documented | 26 |
| LTR41_dup422 | Uniquely | No | — | — | LTR | ERVL | chr3: 114349003-114349702 | − | 110 | 700 | ZBTB20 | Proximal | 0 | Not documented | 27 |
| L1ME2_dup2804 | Multi | No | — | — | LINE | Other L1 | chr3: 114371440-114374084 | − | 120 | 2645 | ZBTB20 | Proximal | 0 | Not documented | 28 |
| L1ME2_dup2805 | Multi/ | No | — | — | LINE | Other L1 | chr3: 114374399-114375581 | − | 120 | 1183 | ZBTB20 | Proximal | 0 | Not documented | 29 |
| | Uniquely | | | | | | | | | | | | | | |
| L1ME2_dup2807 | Uniquely | No | — | — | LINE | Other L1 | chr3: 114376218-114376670 | − | 140 | 453 | ZBTB20 | Proximal | 0 | Not documented | 30 |
| L1ME4a_dup8989 | Multi | No | — | — | LINE | Other L1 | chr3: 114410452-114410734 | + | 130 | 283 | ZBTB20 | Proximal | 0 | Not documented | 31 |
| L2c_dup31619 | Multi | No | — | — | LINE | L2 | chr3: 114410749-114410817 | − | 160 | 69 | ZBTB20 | Proximal | 0 | Not documented | 32 |
| MIR_dup36508 | Multi/ | No | — | — | SINE | MIR | chr3: 114412305-114412398 | + | 160 | 94 | ZBTB20 | Proximal | 0 | Not documented | 33 |
| | Uniquely | | | | | | | | | | | | | | |
| MLT1B_dup3361 | Uniquely | No | — | — | LTR | ERVL- | chr3: 114449269-114449648 | + | 87 | 380 | ZBTB20 | Proximal | 0 | Not documented | 34 |
| | | | | | | MaLR | | | | | | | | | |
| MIR3_dup19690 | Uniquely | No | — | — | SINE | MIR | chr3: 114784514-114784666 | − | 140 | 153 | ZBTB20 | Proximal | 0 | Not documented | 35 |
| L2c_dup31646 | Multi | No | — | — | LINE | L2 | chr3: 114785086-114785592 | + | 160 | 507 | ZBTB20 | Proximal | 0 | Not documented | 36 |
| L2c_dup31700 | Multi/ | No | — | — | LINE | L2 | chr3: 115346992-115348191 | + | 170 | 1200 | GAP43 | Proximal | 0 | Not documented | 37 |
| | Uniquely | | | | | | | | | | | | | | |
| L2c_dup31701 | Multi/ | No | — | — | LINE | L2 | chr3: 115348292-115348593 | + | 150 | 302 | GAP43 | Proximal | 0 | Not documented | 38 |
| | Uniquely | | | | | | | | | | | | | | |
| L2_dup11475 | Uniquely | No | — | — | LINE | L2 | chr3: 115356236-115357293 | + | 160 | 1058 | GAP43 | Proximal | 0 | Not documented | 39 |
| FLAM_C_dup4330 | Multi/ | No | — | — | SINE | Alu | chr3: 115359882-115360011 | − | 66 | 130 | GAP43 | Proximal | 0 | Not documented | 40 |
| | Uniquely | | | | | | | | | | | | | | |
| Charlie15a_dup761 | Multi/ | No | — | — | DNA | DNA | chr3: 115360676-115360797 | − | 130 | 122 | GAP43 | Proximal | 0 | Not documented | 41 |
| | Uniquely | | | | | | | | | | | | | | |
| MIRb_dup48752 | Multi/ | No | — | — | SINE | MIR | chr3: 115383271-115383453 | − | 150 | 183 | GAP43 | Proximal | 0 | Not documented | 42 |
| | Uniquely | | | | | | | | | | | | | | |
| L2a_dup35855 | Multi/ | No | — | — | LINE | L2 | chr3: 115436500-115436598 | + | 130 | 99 | GAP43 | Proximal | 0 | Not documented | 43 |
| | Uniquely | | | | | | | | | | | | | | |
| HERV17-int_dup196 | Uniquely | No | — | — | LTR | ERV1 | chr3: 115463422-115464840 | + | 28 | 1419 | GAP43 | Distal | 23086 | EVE annotated | 44 |
| MIR3_dup19714 | Uniquely | No | — | — | SINE | MIR | chr3: 115523149-115523322 | − | 120 | 174 | LSAMP | Proximal | 0 | Not documented | 45 |
| LTR12 _{——} dup97 | Uniquely | No | — | — | LTR | ERV1 | chr3: 115658717-115659382 | − | 34 | 666 | LSAMP | Proximal | | Not documented | 46 |
| L1PB2_dup526 | Uniquely | No | — | — | LINE | L1PA\|B\|x | chr3: 115668118-115670301 | + | 60 | 2184 | LSAMP | Proximal | 0 | Not documented | 47 |
| L2_dup11488 | Uniquely | No | — | — | LINE | L2 | chr3: 115702546-115703234 | − | 170 | 689 | LSAMP | Proximal | 0 | Not documented | 48 |
| MIRb_dup40780 | Multi | No | — | — | SINE | MIR | chr3: 11579841-11579970 | + | 130 | 130 | ATG7 | Proximal | 0 | Not documented | 49 |
| AluJb_dup23972 | Multi | No | — | — | SINE | Alu | chr3: 11580036-11580369 | + | 75 | 334 | ATG7 | Proximal | 0 | Not documented | 3150 |
| HERVH-int_dup1229 | Multi/ | No | — | — | LTR | ERV1 | chr3: 115826494-115829645 | − | 42 | 3152 | LSAMP | Proximal | 0 | EVE annotated | 51 |
| | Uniquely | | | | | | | | | | | | | | |
| MIR_dup36588 | Uniquely | No | — | — | SINE | MIR | chr3: 115834087-115834312 | − | 130 | 226 | LSAMP | Proximal | 0 | Not documented | 52 |
| CR1_Mam_dup374 | Multi | No | — | — | LINE | CR1 | chr3: 115903666-115904160 | − | 150 | 495 | LSAMP | Proximal | 0 | Not documented | 53 |
| AluY_dup22894 | Multi/ | No | — | — | SINE | Alu | chr3: 115952012-115952281 | + | 27 | 270 | LSAMP | Proximal | 0 | Not documented | 54 |
| | Uniquely | | | | | | | | | | | | | | |
| Tigger10_dup191 | Uniquely | No | — | — | DNA | DNA | chr3: 115977444-115978199 | − | 140 | 756 | LSAMP | Proximal | 0 | Not documented | 55 |
| AluJo_dup13505 | Multi/ | No | — | — | SINE | Alu | chr3: 115993922-115994199 | − | 82 | 278 | LSAMP | Proximal | 0 | Not documented | 56 |
| | Uniquely | | | | | | | | | | | | | | |
| L2a_dup35891 | Uniquely | No | — | — | LINE | L2 | chr3: 116024903-116025035 | − | 150 | 133 | LSAMP | Proximal | 0 | Not documented | 57 |
| L1MB3_dup3438 | Uniquely | No | — | — | LINE | Other L1 | chr3: 116048932-116050675 | + | 79 | 1744 | LSAMP | Proximal | 0 | Not documented | 58 |
| MLT1G3_dup559 | Uniquely | No | — | — | LTR | ERVL- | chr3: 116058344-116058851 | + | 140 | 508 | LSAMP | Proximal | 0 | Not documented | 59 |
| | | | | | | MaLR | | | | | | | | | |
| AluSz_dup18376 | Multi/ | No | — | — | SINE | Alu | chr3: 116065168-116065467 | − | 73 | 300 | LSAMP | Proximal | 0 | Not documented | 60 |
| | Uniquely | | | | | | | | | | | | | | |
| L1MB3_dup3443 | Uniquely | No | — | — | LINE | Other L1 | chr3: 116089592-116092054 | + | 85 | 2463 | LSAMP | Proximal | 0 | Not documented | 61 |
| MIRb_dup48810 | Multi | No | — | — | SINE | MIR | chr3: 116104802-116105006 | − | 120 | 205 | LSAMP | Proximal | 0 | Not documented | 62 |
| L2c_dup31758 | Multi | No | — | — | LINE | L2 | chr3: 116109201-116109419 | − | 180 | 219 | LSAMP | Proximal | 0 | Not documented | 63 |
| AluSc8_dup4137 | Multi | No | — | — | SINE | Alu | chr3: 116109427-116109610 | + | 50 | 184 | LSAMP | Proximal | 0 | Not documented | 64 |
| L3_dup9863 | Uniquely | No | — | — | LINE | CR1 | chr3: 116149894-116150293 | − | 150 | 400 | LSAMP | Proximal | 0 | Not documented | 65 |
| L1ME1_dup6529 | Multi | No | — | — | LINE | Other L1 | chr3: 116264328-116264600 | + | 110 | 273 | LSAMP | Proximal | 0 | Not documented | 66 |
| AluSz_dup18378 | Multi/ | No | — | — | SINE | Alu | chr3: 116264601-116264899 | + | 61 | 299 | LSAMP | Proximal | 0 | Not documented | 67 |
| | Uniquely | | | | | | | | | | | | | | |
| L2c_dup31770 | Multi | No | — | — | LINE | L2 | chr3: 116302187-116302449 | − | 150 | 263 | LSAMP | Proximal | 0 | Not documented | 68 |
| MIR_dup36617 | Multi | No | — | — | SINE | MIR | chr3: 116303024-116303242 | − | 120 | 219 | LSAMP | Proximal | 0 | Not documented | 69 |
| MER102b_dup828 | Uniquely | No | — | — | DNA | DNA | chr3: 116428148-116428425 | − | 150 | 278 | LSAMP | Proximal | 0 | Not documented | 70 |
| MIR_dup36636 | Uniquely | No | — | — | SINE | MIR | chr3: 116617578-116617766 | − | 130 | 189 | LSAMP | Proximal | 0 | Not documented | 71 |
| AluSq10_dup362 | Uniquely | No | — | — | SINE | Alu | chr3: 11682310-11682612 | + | 40 | 303 | VGLL4 | Proximal | 0 | Not documented | 72 |
| AluSx_dup26325 | Multi/ | No | — | — | SINE | Alu | chr3: 116893682-116893987 | + | 42 | 306 | LSAMP | Proximal | 0 | Not documented | 73 |
| | Uniquely | | | | | | | | | | | | | | |
| MER4D1_dup244 | Uniquely | No | — | — | LTR | ERV1 | chr3: 117032941-117033921 | + | 59 | 981 | LSAMP | Proximal | 0 | Not documented | 74 |
| MER103C_dup1975 | Uniquely | No | — | — | DNA | DNA | chr3: 117153804-117153993 | − | 120 | 190 | LSAMP | Proximal | 0 | Not documented | 3175 |
| L2c_dup31827 | Uniquely | No | — | — | LINE | L2 | chr3: 117176048-117176533 | − | 160 | 486 | LSAMP | Proximal | 0 | Not documented | 76 |
| AluJb_dup28286 | Uniquely | No | — | — | SINE | Alu | chr3: 117353579-117353873 | − | 77 | 295 | LSAMP | Proximal | 0 | Not documented | 77 |
| L1MD3_dup1030 | Uniquely | No | — | — | LINE | Other L1 | chr3: 117375395-117375996 | − | 94 | 602 | LSAMP | Proximal | 0 | Not documented | 78 |
| L1P4a_dup89 | Uniquely | No | — | — | LINE | L1PA\|B\|x | chr3: 117666368-117668326 | − | 75 | 1959 | LSAMP | Proximal | 0 | Not documented | 79 |
| MIR3_dup19894 | Multi | No | — | — | SINE | MIR | chr3: 120114638-120114696 | − | 110 | 59 | FSTL1 | Proximal | 0 | Not documented | 80 |
| L1PB4_dup1251 | Uniquely | No | — | — | LINE | L1PA\|B\|x | chr3: 122132838-122133176 | + | 66 | 339 | WDR5B | Proximal | 0 | Not documented | 81 |
| AluJb_dup28474 | Multi | No | — | — | SINE | Alu | chr3: 122292674-122292983 | − | 84 | 310 | DTX3L | Proximal | 0 | Not documented | 82 |
| MIR_dup36970 | Multi | No | — | — | SINE | MIR | chr3: 122293354-122293577 | − | 110 | 224 | DTX3L | Proximal | 0 | Not documented | 83 |
| LTR81A_dup211 | Uniquely | No | — | — | LTR | Gypsy | chr3: 123768657-123769858 | − | 130 | 1202 | KALRN | Distal | 29013 | Not documented | 84 |
| MIRc_dup23279 | Uniquely | No | — | — | SINE | MIR | chr3: 124686464-124686618 | + | 110 | 155 | HEG1 | Proximal | 0 | Not documented | 85 |
| AluSx_dup26647 | Multi/ | No | — | — | SINE | Alu | chr3: 125491830-125492134 | + | 54 | 305 | ALG1L | Distal | 155985 | Not documented | 86 |
| | Uniquely | | | | | | | | | | | | | | |
| HERVK-int_dup43 | Multi/ | No | — | — | LTR | ERVK | chr3: 125610106-125617635 | + | 11 | 7530 | ALG1L | Distal | 30484 | EVE annotated | 87 |
| | Uniquely | | | | | | | | | | | | | | |
| AluSx3_dup5716 | Uniquely | No | — | — | SINE | Alu | chr3: 125869390-125869700 | − | 35 | 311 | ALDH1L1 | Proximal | 0 | Not documented | 88 |
| L2a_dup36393 | Uniquely | No | — | — | LINE | L2 | chr3: 125900228-125900638 | + | 120 | 411 | ALDH1L1 | Proximal | 0 | Not documented | 89 |
| Tigger15a_dup1171 | Uniquely | No | — | — | DNA | DNA | chr3: 126178778-126178929 | − | 120 | 152 | ZXDC | Proximal | 0 | Not documented | 90 |
| MIR3_dup20202 | Multi | No | — | — | SINE | MIR | chr3: 127789993-127790114 | − | 130 | 122 | SEC61A1 | Proximal | 0 | Not documented | 91 |
| L1MC3_dup2437 | Multi | No | — | — | LINE | Other L1 | chr3: 127799004-127799831 | + | 74 | 828 | RUVBL1 | Proximal | 0 | Not documented | 92 |
| L3_dup10133 | Multi/ | No | — | — | LINE | CR1 | chr3: 127823604-127823665 | − | 130 | 62 | RUVBL1 | Proximal | 0 | Not documented | 93 |
| | Uniquely | | | | | | | | | | | | | | |
| AluJb_dup24103 | Uniquely | No | — | — | SINE | Alu | chr3: 12876657-12876916 | + | 72 | 260 | RPL32 | Proximal | 0 | Not documented | 94 |
| MIR3_dup20242 | Multi | No | — | — | SINE | MIR | chr3: 128887076-128887140 | + | 130 | 65 | CNBP | Proximal | 1188 | Not documented | 95 |
| AluSx1_dup20219 | Multi | No | — | — | SINE | Alu | chr3: 128887212-128887472 | + | 49 | 261 | CNBP | Proximal | 856 | Not documented | 96 |
| AluJr4_dup3812 | Multi | No | — | — | SINE | Alu | chr3: 132277343-132277582 | − | 66 | 240 | ACAD11 | Proximal | 0 | Not documented | 97 |
| MER31A_dup292 | Uniquely | No | — | — | LTR | ERV1 | chr3: 133381802-133382205 | + | 66 | 404 | TOPBP1 | Proximal | 1066 | Not documented | 98 |
| Tigger12_dup96 | Multi | No | — | — | DNA | DNA | chr3: 133539108-133539299 | − | 130 | 192 | SRPRB | Proximal | 0 | Not documented | 99 |
| L2b_dup21834 | Multi/ | No | — | — | LINE | L2 | chr3: 134204159-134204329 | + | 170 | 171 | ANAPC13 | Proximal | 0 | Not documented | 3200 |
| | Uniquely | | | | | | | | | | | | | | |
| L3_dup10307 | Multi/ | No | — | — | LINE | CR1 | chr3: 137907290-137907505 | + | 170 | 216 | ARMC8 | Proximal | 0 | Not documented | 1 |
| | Uniquely | | | | | | | | | | | | | | |
| MIRb_dup50734 | Multi | No | — | — | SINE | MIR | chr3: 138123458-138123545 | + | 130 | 88 | MRAS | Proximal | 0 | Not documented | 2 |
| L1MC4a_dup5847 | Uniquely | No | — | — | LINE | Other L1 | chr3: 139324850-139326686 | − | 88 | 1837 | NMNAT3 | Proximal | 0 | Not documented | 3 |
| MER58A_dup2842 | Uniquely | No | — | — | DNA | DNA | chr3: 141012477-141012696 | − | 92 | 220 | ACPL2 | Proximal | 0 | Not documented | 4 |
| MIR_dup30471 | Multi | No | — | — | SINE | MIR | chr3: 14222826-14223071 | − | 87 | 246 | LSM3 | Proximal | 0 | Not documented | 5 |
| L2a_dup37675 | Multi/ | No | — | — | LINE | L2 | chr3: 147713521-147713655 | − | 180 | 135 | ZIC1 | Distal | 485442 | Not documented | 6 |
| | Uniquely | | | | | | | | | | | | | | |
| AluSq_dup4235 | Multi | No | — | — | SINE | Alu | chr3: 149237447-149237764 | + | 45 | 318 | WWTR1 | Proximal | 0 | Not documented | 7 |
| MIRb_dup51667 | Uniquely | No | — | — | SINE | MIR | chr3: 149456277-149456499 | − | 120 | 223 | COMMD2 | Proximal | 0 | Not documented | 8 |
| AluSc8_dup4370 | Multi | No | — | — | SINE | Alu | chr3: 149458826-149459151 | − | 46 | 326 | COMMD2 | Proximal | 0 | Not documented | 9 |
| L2b_dup18022 | Multi | No | — | — | LINE | L2 | chr3: 14984926-14985007 | − | 130 | 82 | NR2C2 | Distal | 4085 | Not documented | 10 |
| MER5B_dup4073 | Multi | No | — | — | DNA | DNA | chr3: 14985199-14985301 | + | 150 | 103 | NR2C2 | Distal | 3791 | Not documented | 11 |
| MIRb_dup41355 | Multi/ | No | — | — | SINE | MIR | chr3: 14985746-14985918 | + | 160 | 173 | NR2C2 | Distal | 3174 | Not documented | 12 |
| | Uniquely | | | | | | | | | | | | | | |
| AluSz_dup19357 | Multi/ | No | — | — | SINE | Alu | chr3: 150180412-150180724 | − | 30 | 313 | TSC22D2 | Proximal | 0 | Not documented | 13 |
| | Uniquely | | | | | | | | | | | | | | |
| L2c_dup33508 | Multi | No | — | — | LINE | L2 | chr3: 150182256-150182341 | − | 120 | 86 | TSC22D2 | Proximal | 0 | Not documented | 14 |
| L1MC5_dup4364 | Uniquely | No | — | — | LINE | Other L1 | chr3: 150282489-150283355 | − | 100 | 867 | EIF2A | Proximal | 0 | Not documented | 15 |
| Tigger4a_dup752 | Multi | No | — | — | DNA | DNA | chr3: 150346668-150346844 | − | 50 | 177 | SELT | Proximal | 0 | Not documented | 16 |
| L2c_dup27476 | Uniquely | No | — | — | LINE | L2 | chr3: 15138171-15138264 | − | 150 | 94 | ZFYVE20 | Proximal | 0 | Not documented | 17 |
| L1PBa_dup452 | Uniquely | No | — | — | LINE | L1PA\|B\|x | chr3: 152406045-152411911 | + | 46 | 5867 | PZRY1 | Distal | 140826 | Not documented | 18 |
| MER61B_dup55 | Multi | No | — | — | LTR | ERV1 | chr3: 153784-154149 | − | 88 | 366 | CHL1 | Distal | 84131 | Not documented | 19 |
| AluSp_dup10013 | Uniquely | No | — | — | SINE | Alu | chr3: 153837318-153837611 | + | 39 | 294 | ARHGEF26 | Proximal | 1182 | Not documented | 20 |
| AluSz_dup19462 | Multi/ | No | — | — | SINE | Alu | chr3: 153974044-153974321 | − | 55 | 278 | ARHGEF26 | Proximal | 0 | Not documented | 21 |
| | Uniquely | | | | | | | | | | | | | | |
| FLAM_C_dup3771 | Multi | No | — | — | SINE | Alu | chr3: 15483062-15483191 | + | 56 | 130 | EAF1 | Proximal | 0 | Not documented | 22 |
| AluSx1_dup20965 | Multi | No | — | — | SINE | Alu | chr3: 156259207-156259414 | − | 50 | 208 | SSR3 | Proximal | 0 | Not documented | 23 |
| Charlie17a_dup356 | Multi | No | — | — | DNA | DNA | chr3: 156259896-156260002 | + | 88 | 107 | SSR3 | Proximal | 0 | Not documented | 24 |
| AluJr4_dup3944 | Multi | No | — | — | SINE | Alu | chr3: 156417879-156418190 | − | 63 | 312 | TIPARP | Proximal | 0 | Not documented | 3225 |
| AluY_dup24364 | Multi | No | — | — | SINE | Alu | chr3: 158262979-158263210 | − | 33 | 232 | RSRC1 | Proximal | 0 | Not documented | 26 |
| L1MCa_dup1432 | Uniquely | No | — | — | LINE | Other L1 | chr3: 158298752-158299684 | + | 69 | 933 | MLF1 | Proximal | 0 | Not documented | 27 |
| Tigger14a_dup291 | Multi | No | — | — | DNA | DNA | chr3: 160803013-160803101 | + | 120 | 89 | B3GALNT1 | Proximal | 0 | Not documented | 28 |
| AluSx_dup22761 | Multi/ | No | — | — | SINE | Alu | chr3: 16269867-16270170 | + | 52 | 304 | GALNT15 | Proximal | 0 | Not documented | 29 |
| | Uniquely | | | | | | | | | | | | | | |
| MER3_dup1803 | Uniquely | No | — | — | DNA | DNA | chr3: 16270775-16270974 | − | 82 | 200 | GALNT15 | Proximal | 0 | Not documented | 30 |
| MLT2B4_dup942 | Uniquely | No | — | — | LTR | ERVL | chr3: 165177948-165178499 | + | 79 | 552 | SLITRK3 | Distal | 263052 | Not documented | 31 |
| L1MA4_dup2101 | Uniquely | No | — | — | LINE | Other L1 | chr3: 168447949-168453738 | + | 87 | 5790 | MECOM | Distal | 347550 | Not documented | 32 |
| MER82_dup664 | Multi | No | — | — | DNA | DNA | chr3: 169713456-169713984 | + | 77 | 529 | SEC62 | Proximal | 0 | Not documented | 33 |
| HERVH-int_dup1416 | Uniquely | No | — | — | LTR | ERV1 | chr3: 170403448-170406593 | − | 44 | 3146 | CLDN11 | Proximal | 0 | EVE annotated | 34 |
| MER58B_dup1516 | Uniquely | No | — | — | DNA | DNA | chr3: 171040654-171040990 | + | 110 | 337 | TNIK | Proximal | 0 | Not documented | 35 |
| L2a_dup38981 | Uniquely | No | — | — | LINE | L2 | chr3: 171063572-171063992 | − | 130 | 421 | TNIK | Proximal | 0 | Not documented | 36 |
| L2b_dup23002 | Multi/ | No | — | — | LINE | L2 | chr3: 171064250-171064971 | − | 140 | 722 | TNIK | Proximal | 0 | Not documented | 37 |
| | Uniquely | | | | | | | | | | | | | | |
| AluY_dup24895 | Multi | No | — | — | SINE | Alu | chr3: 173599210-173599486 | − | 26 | 277 | NLGN1 | Proximal | 0 | Not documented | 38 |
| LTR86B2_dup67 | Uniquely | No | — | — | LTR | ERVL | chr3: 173622552-173622996 | − | 140 | 445 | NLGN1 | Proximal | 0 | Not documented | 39 |
| AluSz_dup19897 | Multi | No | — | — | SINE | Alu | chr3: 173646463-173646759 | − | 52 | 297 | NLGN1 | Proximal | 0 | Not documented | 40 |
| LTR78_dup1150 | Uniquely | No | — | — | LTR | ERV1 | chr3: 173689577-173690165 | + | 150 | 589 | NLGN1 | Proximal | 0 | Not documented | 41 |
| MLT1L_dup2777 | Multi/ | No | — | — | LTR | ERVL- | chr3: 173707411-173707906 | − | 140 | 496 | NLGN1 | Proximal | 0 | Not documented | 42 |
| | Uniquely | | | | | MaLR | | | | | | | | | |
| MLT1B_dup3715 | Uniquely | No | — | — | LTR | ERVL- | chr3: 173724877-173725122 | + | 61 | 246 | NLGN1 | Proximal | 0 | Not documented | 43 |
| | | | | | | MaLR | | | | | | | | | |
| HAL1_dup6411 | Multi | No | — | — | LINE | Other L1 | chr3: 173748374-173750069 | + | 130 | 1696 | NLGN1 | Proximal | 0 | Not documented | 44 |
| L1MCa_dup1488 | Multi | No | — | — | LINE | Other L1 | chr3: 173750832-173752865 | + | 99 | 2034 | NLGN1 | Proximal | 0 | Not documented | 45 |
| L1PA16_dup2930 | Uniquely | No | — | — | LINE | L1PA\|B\|x | chr3: 173881222-173888019 | − | 75 | 6798 | NLGN1 | Proximal | 0 | Not documented | 46 |
| L1MA4_dup2134 | Uniquely | No | — | — | LINE | Other L1 | chr3: 174568122-174570873 | + | 74 | 2752 | NAALADL2 | Proximal | 0 | Not documented | 47 |
| L1MA1_dup859 | Uniquely | No | — | — | LINE | Other L1 | chr3: 174609036-174610439 | + | 85 | 1404 | NAALADL2 | Proximal | 0 | Not documented | 48 |
| L1PA4_dup2527 | Uniquely | No | — | — | LINE | L1PA\|B\|x | chr3: 175141055-175142530 | − | 18 | 1476 | NAALADL2 | Proximal | 0 | Not documented | 49 |
| MIRb_dup53253 | Multi/ | No | — | — | SINE | MIR | chr3: 175149862-175150068 | + | 160 | 207 | NAALADL2 | Proximal | 0 | Not documented | 3250 |
| | Uniquely | | | | | | | | | | | | | | |
| AluY_dup25017 | Uniquely | No | — | — | SINE | Alu | chr3: 177062160-177062469 | − | 30 | 310 | TBL1XR1 | Distal | 146900 | Not documented | 51 |
| L1MA2_dup1506 | Uniquely | No | — | — | LINE | Other L1 | chr3: 178606553-178610586 | + | 52 | 4034 | KCNMB2 | Distal | 44337 | Not documented | 52 |
| MIRc_dup25131 | Multi | No | — | — | SINE | MIR | chr3: 179050749-179050902 | − | 120 | 154 | ZNF639 | Proximal | 0 | Not documented | 53 |
| AluJb_dup30685 | Uniquely | No | — | — | SINE | Alu | chr3: 180701721-180701822 | + | 80 | 102 | DNAJC19 | Proximal | 0 | Not documented | 54 |
| L2c_dup35003 | Multi/ | No | — | — | LINE | L2 | chr3: 180702049-180702146 | + | 110 | 98 | DNAJC19 | Proximal | 0 | Not documented | 55 |
| | Uniquely | | | | | | | | | | | | | | |
| L2a_dup39571 | Multi | No | — | — | LINE | L2 | chr3: 181339430-181339689 | − | 130 | 260 | SOX2 | Distal | 90026 | Not documented | 56 |
| L2c_dup35056 | Multi | No | — | — | LINE | L2 | chr3: 181340463-181340711 | + | 150 | 249 | SOX2 | Distal | 89004 | Not documented | 57 |
| AluJo_dup14714 | Multi | No | — | — | SINE | Alu | chr3: 181341007-181341086 | + | 96 | 80 | SOX2 | Distal | 88629 | Not documented | 58 |
| Charlie1a_dup1319 | Uniquely | No | — | — | DNA | DNA | chr3: 181389388-181390529 | + | 84 | 1142 | SOX2 | Distal | 39186 | Not documented | 59 |
| AluSx_dup28803 | Multi | No | — | — | SINE | Alu | chr3: 183529740-183530027 | + | 44 | 288 | YEATS2 | Proximal | 0 | Not documented | 60 |
| L2a_dup39724 | Uniquely | No | — | — | LINE | L2 | chr3: 184018480-184018760 | + | 160 | 281 | PSMD2 | Proximal | 0 | Not documented | 61 |
| AluY_dup25374 | Multi | No | — | — | SINE | Alu | chr3: 184796788-184797092 | + | 34 | 305 | C3orf70 | Proximal | 0 | Not documented | 62 |
| AluSx1_dup21785 | Multi | No | — | — | SINE | Alu | chr3: 185009811-185010106 | + | 48 | 296 | MAP3K13 | Proximal | 0 | Not documented | 63 |
| MIRc_dup25375 | Multi | No | — | — | SINE | MIR | chr3: 185010173-185010412 | + | 120 | 240 | MAP3K13 | Proximal | 0 | Not documented | 64 |
| MER112_dup1127 | Multi | No | — | — | DNA | DNA | chr3: 185010664-185010871 | + | 110 | 208 | MAP3K13 | Proximal | 0 | Not documented | 65 |
| AluY_dup25449 | Multi | No | — | — | SINE | Alu | chr3: 186264337-186264647 | + | 23 | 311 | TBCCD1 | Proximal | 0 | Not documented | 66 |
| MIR3_dup21984 | Multi/ | No | — | — | SINE | MIR | chr3: 186952194-186952364 | − | 120 | 171 | MASP1 | Proximal | 0 | Not documented | 67 |
| | Uniquely | | | | | | | | | | | | | | |
| L3_dup11020 | Uniquely | No | — | — | LINE | CR1 | chr3: 186952945-186953083 | + | 130 | 139 | MASP1 | Proximal | 0 | Not documented | 68 |
| Ricksha_0_dup103 | Uniquely | No | — | — | DNA | DNA | chr3: 187013697-187015275 | + | 63 | 1579 | MASP1 | Distal | 3888 | Not documented | 69 |
| MIRc_dup25546 | Multi/ | No | — | — | SINE | MIR | chr3: 187453116-187453240 | − | 140 | 125 | BCL6 | Proximal | 0 | Not documented | 70 |
| | Uniquely | | | | | | | | | | | | | | |
| MIR3_dup22054 | Multi | No | — | — | SINE | MIR | chr3: 187455906-187455971 | − | 54 | 66 | BCL6 | Proximal | 0 | Not documented | 71 |
| MIR3_dup22162 | Multi | No | — | — | SINE | MIR | chr3: 187943131-187943215 | − | 92 | 85 | LPP | Proximal | 0 | Not documented | 72 |
| MIR3_dup22176 | Uniquely | No | — | — | SINE | MIR | chr3: 188022085-188022258 | − | 120 | 174 | LPP | Proximal | 0 | Not documented | 73 |
| AluJr_dup15598 | Multi | No | — | — | SINE | Alu | chr3: 188595352-188595525 | + | 60 | 174 | LPP | Proximal | 0 | Not documented | 74 |
| AluY_dup25655 | Multi/ | No | — | — | SINE | Alu | chr3: 193117366-193117656 | + | 35 | 291 | ATP13A4 | Distal | 2211 | Not documented | 3275 |
| | Uniquely | | | | | | | | | | | | | | |
| L1MC2_dup1353 | Uniquely | No | — | — | LINE | Other L1 | chr3: 193150002-193150527 | + | 76 | 526 | ATP13A4 | Proximal | 0 | Not documented | 76 |
| L1MC1_dup2968 | Multi | No | — | — | LINE | Other L1 | chr3: 193150962-193151123 | + | 89 | 162 | ATP13A4 | Proximal | 0 | Not documented | 77 |
| AluY_dup25658 | Uniquely | No | — | — | SINE | Alu | chr3: 193155615-193155920 | − | 35 | 306 | ATP13A4 | Proximal | 0 | Not documented | 78 |
| FLAM_C_dup4743 | Uniquely | No | — | — | SINE | Alu | chr3: 193180652-193180786 | + | 67 | 135 | ATP13A4 | Proximal | 0 | Not documented | 79 |
| MIRc_dup26094 | Uniquely | No | — | — | SINE | MIR | chr3: 193180943-193181095 | + | 140 | 153 | ATP13A4 | Proximal | 0 | Not documented | 80 |
| MSTA_dup4116 | Multi | No | — | — | LTR | ERVL- | chr3: 194361500-194361861 | − | 67 | 362 | LSG1 | Proximal | 0 | Not documented | 81 |
| | | | | | | MaLR | | | | | | | | | |
| L1MB8_dup3496 | Multi | No | — | — | LINE | Other L1 | chr3: 194361888-194362131 | − | 75 | 244 | LSG1 | Proximal | 0 | Not documented | 82 |
| AluSx3_dup6321 | Multi | No | — | — | SINE | Alu | chr3: 194362350-194362551 | − | 40 | 202 | LSG1 | Proximal | 0 | Not documented | 83 |
| L1M1_dup2051 | Uniquely | No | — | — | LINE | Other L1 | chr3: 195076666-195078462 | + | 74 | 1797 | ACAP2 | Proximal | 0 | Not documented | 84 |
| L2b_dup23899 | Multi | No | — | — | LINE | L2 | chr3: 195388563-195388684 | + | 120 | 122 | MUC20 | Distal | 59070 | Not documented | 85 |
| L2a_dup40352 | Uniquely | No | — | — | LINE | L2 | chr3: 195461911-195462761 | + | 130 | 851 | MUC20 | Proximal | 0 | Not documented | 86 |
| LTR5_Hs_dup150 | Multi | No | — | — | LTR | ERVK | chr3: 195654396-195655363 | − | 5 | 968 | TNK2 | Distal | 15581 | Not documented | 87 |
| AluSq2_dup11657 | Uniquely | No | — | — | SINE | Alu | chr3: 196433901-196434200 | − | 27 | 300 | CEP19 | Proximal | 0 | Not documented | 88 |
| AluSx1_dup22393 | Multi/ | No | — | — | SINE | Alu | chr3: 196461161-196461462 | + | 60 | 302 | PIGX | Proximal | 0 | Not documented | 89 |
| | Uniquely | | | | | | | | | | | | | | |
| AluSx_dup29723 | Multi | No | — | — | SINE | Alu | chr3: 196660479-196660772 | − | 43 | 294 | SENP5 | Proximal | 0 | Not documented | 90 |
| AluJr_dup15787 | Multi | No | — | — | SINE | Alu | chr3: 196663325-196663446 | − | 52 | 122 | NCBP2 | Proximal | 0 | Not documented | 91 |
| AluJb_dup31408 | Uniquely | No | — | — | SINE | Alu | chr3: 196670492-196670604 | − | 48 | 113 | NCBP2 | Proximal | 1025 | Not documented | 92 |
| L1M4c_dup1228 | Multi | No | — | — | LINE | Other L1 | chr3: 196883869-196884764 | + | 88 | 896 | DLG1 | Proximal | 0 | Not documented | 93 |
| L1M4c_dup1229 | Multi | No | — | — | LINE | Other L1 | chr3: 196884765-196885210 | + | 85 | 446 | DLG1 | Proximal | 0 | Not documented | 94 |
| MER20_dup3715 | Uniquely | No | — | — | DNA | DNA | chr3: 196955859-196956062 | + | 110 | 204 | DLG1 | Proximal | 0 | Not documented | 95 |
| Tigger3b_dup1502 | Uniquely | No | — | — | DNA | DNA | chr3: 196995509-196996270 | − | 72 | 762 | DLG1 | Proximal | 0 | Not documented | 96 |
| LTR8A_dup593 | Multi | No | — | — | LTR | ERV1 | chr3: 197340125-197340848 | − | 70 | 724 | BDH1 | Distal | 39932 | Not documented | 97 |
| MER4-int_dup577 | Multi | No | — | — | LTR | ERV1 | chr3: 197341324-197342796 | − | 44 | 1473 | BDH1 | Distal | 41131 | Not documented | 98 |
| AluJo_dup15104 | Uniquely | No | — | — | SINE | Alu | chr3: 197682860-197683121 | − | 89 | 262 | RPL35A | Proximal | 0 | Not documented | 99 |
| MER113_dup361 | Multi/ | No | — | — | DNA | DNA | chr3: 254555-254908 | − | 140 | 354 | CHL1 | Proximal | 0 | Not documented | 3300 |
| | Uniquely | | | | | | | | | | | | | | |
| MLT1H2_dup810 | Multi | No | — | — | LTR | ERVL- | chr3: 25508724-25509184 | − | 120 | 461 | RARB | Proximal | 0 | Not documented | 1 |
| | | | | | | MaLR | | | | | | | | | |
| L1MA6_dup800 | Uniquely | No | — | — | LINE | Other L1 | chr3: 256746-257365 | + | 72 | 620 | CHL1 | Proximal | 0 | Not documented | 2 |
| MIRb_dup42160 | Multi | No | — | — | SINE | MIR | chr3: 25778598-25778829 | − | 120 | 232 | NGLY1 | Proximal | 0 | Not documented | 3 |
| AluSz_dup16049 | Multi | No | — | — | SINE | Alu | chr3: 27675934-27676257 | + | 46 | 324 | EOMES | Distal | 81184 | Not documented | 4 |
| MER102a_dup384 | Uniquely | No | — | — | DNA | DNA | chr3: 304266-304563 | − | 150 | 298 | CHL1 | Proximal | 0 | Not documented | 5 |
| L2a_dup31099 | Multi | No | — | — | LINE | L2 | chr3: 33905589-33905643 | + | 83 | 55 | PDCD6IP | Proximal | 0 | Not documented | 6 |
| MER20B_dup758 | Multi | No | — | — | DNA | DNA | chr3: 37031148-37031317 | − | 120 | 170 | EPM2AIP1 | Proximal | 0 | Not documented | 7 |
| L1PA5_dup1667 | Uniquely | No | — | — | LINE | L1PA\|B\|x | chr3: 3907117-3910573 | + | 31 | 3457 | SUMF1 | Proximal | 0 | Not documented | 8 |
| LTR6A_dup37 | Multi | No | — | — | LTR | ERV1 | chr3: 39449151-39449180 | − | 15 | 30 | RPSA | Proximal | 0 | Not documented | 9 |
| L2a_dup31454 | Uniquely | No | — | — | LINE | L2 | chr3: 40491283-40491929 | + | 100 | 647 | RPL14 | Distal | 6855 | Not documented | 10 |
| L3_dup8184 | Multi | No | — | — | LINE | CR1 | chr3: 430900-430989 | + | 110 | 90 | CHL1 | Proximal | 0 | Not documented | 11 |
| AluSz6_dup7880 | Uniquely | No | — | — | SINE | Alu | chr3: 43749121-43749421 | − | 66 | 301 | ABHD5 | Proximal | 0 | Not documented | 12 |
| X7B_LINE_dup168 | Multi/ | No | — | — | LINE | CR1 | chr3: 43760330-43760437 | − | 110 | 108 | ABHD5 | Proximal | 0 | Not documented | 13 |
| | Uniquely | | | | | | | | | | | | | | |
| MER2_dup1674 | Uniquely | No | — | — | DNA | DNA | chr3: 44399883-44400215 | + | 69 | 333 | TCAIM | Proximal | 0 | Not documented | 14 |
| L1ME3C_dup2252 | Multi | No | — | — | LINE | Other L1 | chr3: 44547421-44548197 | + | 130 | 777 | ZNF852 | Proximal | 0 | Not documented | 15 |
| L1MD2_dup1789 | Uniquely | No | — | — | LINE | Other L1 | chr3: 44688271-44689546 | + | 81 | 1276 | ZNF197 | Proximal | 0 | Not documented | 16 |
| Tigger3_dup171 | Uniquely | No | — | — | DNA | DNA | chr3: 44722341-44724300 | − | 49 | 1960 | ZNF35 | Distal | 20059 | Not documented | 17 |
| LOR1-int_dup316 | Multi/ | No | — | — | LTR | ERV1 | chr3: 44792801-44793483 | + | 110 | 683 | KIAA1143 | Proximal | 0 | EVE annotated | 18 |
| | Uniquely | | | | | | | | | | | | | | |
| L1ME4a_dup8203 | Multi | No | — | — | LINE | Other L1 | chr3: 44794427-44794772 | + | 110 | 346 | KIAA1143 | Proximal | 0 | Not documented | 19 |
| MIRc_dup18784 | Multi | No | — | — | SINE | MIR | chr3: 450031-450230 | + | 160 | 200 | CHL1 | Proximal | 0 | Not documented | 20 |
| MIR3_dup18057 | Uniquely | No | — | — | SINE | MIR | chr3: 47323607-47323678 | + | 110 | 72 | KIF9 | Proximal | 0 | Not documented | 21 |
| Tigger9a_dup209 | Multi | No | — | — | DNA | DNA | chr3: 47857595-47857760 | − | 110 | 166 | DHX30 | Proximal | 0 | Not documented | 22 |
| L3_dup8974 | Multi | No | — | — | LINE | CR1 | chr3: 47893620-47893695 | + | 140 | 76 | MAP4 | Proximal | 0 | Not documented | 23 |
| L1MD3_dup933 | Uniquely | No | — | — | LINE | Other L1 | chr3: 48123061-48124014 | + | 91 | 954 | MAP4 | Proximal | 0 | Not documented | 24 |
| AluSg_dup7161 | Multi | No | — | — | SINE | Alu | chr3: 48335573-48335861 | − | 36 | 289 | NME6 | Proximal | 0 | Not documented | 3325 |
| AluSc8_dup3761 | Multi | No | — | — | SINE | Alu | chr3: 48962120-48962426 | − | 45 | 307 | ARIH2 | Proximal | 0 | Not documented | 26 |
| AluSg_dup7219 | Multi/ | No | — | — | SINE | Alu | chr3: 49452668-49452957 | + | 44 | 290 | TCTA | Proximal | 0 | Not documented | 27 |
| | Uniquely | | | | | | | | | | | | | | |
| MIR3_dup18119 | Multi | No | — | — | SINE | MIR | chr3: 50257859-50257993 | − | 130 | 135 | GNAI2 | Distal | 5732 | Not documented | 28 |
| AluSz_dup17013 | Multi | No | — | — | SINE | Alu | chr3: 51701186-51701479 | − | 46 | 294 | TEX264 | Proximal | 0 | Not documented | 29 |
| L1MA8_dup1661 | Multi | No | — | — | LINE | Other L1 | chr3: 51982586-51983078 | − | 67 | 493 | PARP3 | Proximal | 0 | Not documented | 30 |
| L2b_dup19214 | Multi | No | — | — | LINE | L2 | chr3: 52568839-52568894 | − | 140 | 56 | NT5DC2 | Proximal | 0 | Not documented | 31 |
| AluJb_dup26364 | Multi | No | — | — | SINE | Alu | chr3: 53850868-53851151 | − | 55 | 284 | CHDH | Proximal | 0 | Not documented | 32 |
| Tigger2_dup550 | Multi | No | — | — | DNA | DNA | chr3: 53894643-53897334 | + | 55 | 2692 | IL17RB | Proximal | 0 | Not documented | 33 |
| AluY_dup21186 | Multi | No | — | — | SINE | Alu | chr3: 56654899-56655216 | + | 46 | 318 | FAM208A | Proximal | 0 | Not documented | 34 |
| L1PA5_dup1875 | Uniquely | No | — | — | LINE | L1PA\|B\|x | chr3: 57592609-57598129 | + | 25 | 5521 | ARF4 | Distal | 8663 | EVE annotated | 35 |
| AluSz_dup17323 | Multi | No | — | — | SINE | Alu | chr3: 58304103-58304410 | + | 74 | 308 | RPP14 | Proximal | 0 | Not documented | 36 |
| MLT1J_dup2835 | Multi/ | No | — | — | LTR | ERVL- | chr3: 58549790-58550158 | − | 140 | 369 | FAM107A | Proximal | 0 | Not documented | 37 |
| | Uniquely | | | | | MaLR | | | | | | | | | |
| Charlie19a_dup319 | Multi/ | No | — | — | DNA | DNA | chr3: 58551051-58551263 | + | 120 | 213 | FAM107A | Proximal | 0 | Not documented | 38 |
| | Uniquely | | | | | | | | | | | | | | |
| MER5A1_dup2999 | Multi/ | No | — | — | DNA | DNA | chr3: 58551475-58551627 | + | 80 | 153 | FAM107A | Proximal | 0 | Not documented | 39 |
| | Uniquely | | | | | | | | | | | | | | |
| MIRb_dup44739 | Multi | No | — | — | SINE | MIR | chr3: 58562456-58562688 | − | 130 | 233 | FAM107A | Proximal | 0 | Not documented | 40 |
| L1PA16_dup2301 | Uniquely | No | — | — | LINE | L1PA\|B\|x | chr3: 60799904-60802547 | − | 58 | 2644 | FHIT | Proximal | 0 | Not documented | 41 |
| L1MB4_dup1703 | Uniquely | No | — | — | LINE | Other L1 | chr3: 64163109-64164000 | − | 81 | 892 | PRICKLE2 | Proximal | 0 | Not documented | 42 |
| L1MB7_dup4328 | Uniquely | No | — | — | LINE | Other L1 | chr3: 66437131-66438239 | + | 100 | 1109 | LRIG1 | Proximal | 0 | Not documented | 43 |
| AluSz_dup17538 | Multi/ | No | — | — | SINE | Alu | chr3: 66535297-66535602 | + | 73 | 306 | LRIG1 | Proximal | 0 | Not documented | 44 |
| | Uniquely | | | | | | | | | | | | | | |
| MER5A_dup6715 | Multi | No | — | — | DNA | DNA | chr3: 68194587-68194689 | − | 130 | 103 | FAM19A1 | Proximal | 0 | Not documented | 45 |
| CER_dup9 | Multi | No | — | — | Other | Other | chr3: 75798061-75803252 | + | 45 | 5192 | ZNF717 | Proximal | 0 | Not documented | 46 |
| | | | | | | Repeats | | | | | | | | | |
| L1MC1_dup2442 | Uniquely | No | — | — | LINE | Other L1 | chr3: 77286714-77287560 | − | 85 | 847 | ROBO2 | Proximal | 0 | Not documented | 47 |
| L1P3_dup476 | Uniquely | No | — | — | LINE | L1PA\|B\|x | chr3: 7945182-7948486 | − | 49 | 3305 | GRM7 | Distal | 161968 | Not documented | 48 |
| AluJb_dup23765 | Uniquely | No | — | — | SINE | Alu | chr3: 8329168-8329479 | + | 76 | 312 | LMCD1 | Distal | 213915 | Not documented | 49 |
| AluJb_dup27725 | Multi | No | — | — | SINE | Alu | chr3: 93700444-93700739 | + | 63 | 296 | ARL13B | Proximal | 0 | Not documented | 3350 |
| AluSp_dup9249 | Multi | No | — | — | SINE | Alu | chr3: 94224673-94224973 | − | 38 | 301 | NSUN3 | Distal | 377285 | Not documented | 51 |
| Mam_R4_dup133 | Multi | No | — | — | LINE | Dong-R4 | chr3: 94225397-94225563 | + | 170 | 167 | NSUN3 | Distal | 378009 | Not documented | 52 |
| Charlie1_dup373 | Multi/ | No | — | — | DNA | DNA | chr3: 9431502-9432125 | − | 93 | 624 | THUMPD3 | Distal | 3028 | Not documented | 53 |
| | Uniquely | | | | | | | | | | | | | | |
| AluSz_dup15533 | Multi | No | — | — | SINE | Alu | chr3: 9432941-9433241 | − | 59 | 301 | THUMPD3 | Distal | 4467 | Not documented | 54 |
| MLT1K_dup2832 | Multi | No | — | — | LTR | ERVL- | chr3: 9807706-9808055 | + | 96 | 350 | CAMK1 | Proximal | 0 | Not documented | 55 |
| | | | | | | MaLR | | | | | | | | | |
| MIR_dup29989 | Multi/ | No | — | — | SINE | MIR | chr3: 9833868-9833965 | + | 88 | 98 | TADA3 | Proximal | 0 | Not documented | 56 |
| | Uniquely | | | | | | | | | | | | | | |
| L1PA5_dup2041 | Multi | No | — | — | LINE | L1PA\|B\|x | chr3: 98520545-98523821 | − | 21 | 3277 | ST3GAL6 | Proximal | 0 | EVE annotated | 57 |
| MIRb_dup40381 | Multi | No | — | — | SINE | MIR | chr3: 9879496-9879669 | − | 130 | 174 | TTLL3 | Proximal | 0 | Not documented | 58 |
| FLAM_C_dup3695 | Multi | No | — | — | SINE | Alu | chr3: 9883324-9883451 | − | 67 | 128 | RPUSD3 | Proximal | 0 | Not documented | 59 |
| L1PA6_dup1621 | Uniquely | No | — | — | LINE | L1PA\|B\|x | chr4: 100182019-100185432 | − | 47 | 3414 | ADH1A | Distal | 12093 | Not documented | 60 |
| MLT1J_dup4214 | Multi | No | — | — | LTR | ERVL- | chr4: 103339851-103340171 | − | 130 | 321 | SLC39A8 | Proximal | 0 | Not documented | 61 |
| | | | | | | MaLR | | | | | | | | | |
| AluY_dup29287 | Multi | No | — | — | SINE | Alu | chr4: 103352220-103352531 | + | 18 | 312 | SLC39A8 | Proximal | 0 | Not documented | 62 |
| MER5A_dup9473 | Multi | No | — | — | DNA | DNA | chr4: 103715272-103715442 | − | 120 | 171 | UBE2D3 | Proximal | 99 | Not documented | 63 |
| MLT1A0_dup5633 | Uniquely | No | — | — | LTR | ERVL- | chr4: 104000229-104000589 | + | 89 | 361 | SLC9B2 | Proximal | 0 | Not documented | 64 |
| | | | | | | MaLR | | | | | | | | | |
| L2c_dup41156 | Uniquely | No | — | — | LINE | L2 | chr4: 104007110-104007307 | + | 160 | 198 | BDH2 | Proximal | 0 | Not documented | 65 |
| Plat_L3_dup1077 | Multi | No | — | — | LINE | CR1 | chr4: 106891547-106891759 | + | 120 | 213 | NPNT | Proximal | 0 | Not documented | 66 |
| AluSc_dup8565 | Uniquely | No | — | — | SINE | Alu | chr4: 108799060-108799369 | − | 44 | 310 | SGMS2 | Proximal | 0 | Not documented | 67 |
| MER4-int_dup741 | Uniquely | No | — | — | LTR | ERV1 | chr4: 108807663-108811549 | − | 60 | 3887 | SGMS2 | Proximal | 0 | Not documented | 68 |
| LTR9_dup450 | Uniquely | No | — | — | LTR | ERV1 | chr4: 108812642-108813255 | − | 96 | 614 | SGMS2 | Proximal | 0 | Not documented | 69 |
| AluSp_dup12189 | Multi | No | — | — | SINE | Alu | chr4: 110610138-110610435 | − | 40 | 298 | CASP6 | Proximal | 0 | Not documented | 70 |
| AluSz_dup23539 | Multi/ | No | — | — | SINE | Alu | chr4: 114371295-114371580 | + | 46 | 286 | CAMK2D | Proximal | 609 | Not documented | 71 |
| | Uniquely | | | | | | | | | | | | | | |
| MIRb_dup63519 | Multi | No | — | — | SINE | MIR | chr4: 114372562-114372749 | − | 140 | 188 | CAMK2D | Proximal | 0 | Not documented | 72 |
| L2c_dup41682 | Multi | No | — | — | LINE | L2 | chr4: 114372791-114373231 | − | 160 | 441 | CAMK2D | Proximal | 0 | Not documented | 73 |
| L2a_dup48098 | Uniquely | No | — | — | LINE | L2 | chr4: 114509883-114511102 | − | 130 | 1220 | CAMK2D | Proximal | 0 | Not documented | 74 |
| MER34A1_dup203 | Uniquely | No | — | — | LTR | ERV1 | chr4: 114523482-114524032 | + | 79 | 551 | CAMK2D | Proximal | 0 | Not documented | 3375 |
| L2c_dup41697 | Uniquely | No | — | — | LINE | L2 | chr4: 114604590-114604884 | − | 170 | 295 | CAMK2D | Proximal | 0 | Not documented | 76 |
| AluY_dup29631 | Multi/ | No | — | — | SINE | Alu | chr4: 114626216-114626504 | + | 36 | 289 | CAMK2D | Proximal | 0 | Not documented | 77 |
| | Uniquely | | | | | | | | | | | | | | |
| MER34_dup398 | Uniquely | No | — | — | LTR | ERV1 | chr4: 114635594-114636056 | + | 88 | 463 | CAMK2D | Proximal | 0 | Not documented | 78 |
| AluSz6_dup11317 | Uniquely | No | — | — | SINE | Alu | chr4: 114644382-114644679 | − | 53 | 298 | CAMK2D | Proximal | 0 | Not documented | 79 |
| L1MDb_dup251 | Multi | No | — | — | LINE | Other L1 | chr4: 114682895-114683011 | + | 94 | 117 | CAMK2D | Proximal | 0 | Not documented | 80 |
| LTR82A_dup277 | Multi | No | — | — | LTR | ERVL | chr4: 120218140-120218583 | − | 130 | 444 | C4orf3 | Proximal | 0 | Not documented | 81 |
| LTR82A_dup278 | Multi | No | — | — | LTR | ERVL | chr4: 120218941-120219016 | − | 110 | 76 | C4orf3 | Proximal | 0 | Not documented | 82 |
| L2a_dup48462 | Multi | No | — | — | LINE | L2 | chr4: 120219047-120219143 | + | 70 | 97 | C4orf3 | Proximal | 0 | Not documented | 83 |
| LTR84b_dup373 | Multi | No | — | — | LTR | ERVL | chr4: 120384050-120384127 | + | 110 | 78 | PDE5A | Distal | 31424 | Not documented | 84 |
| LTR8A_dup686 | Uniquely | No | — | — | LTR | ERV1 | chr4: 123755771-123756453 | + | 76 | 683 | FGF2 | Proximal | 0 | Not documented | 85 |
| L2c_dup42057 | Uniquely | No | — | — | LINE | L2 | chr4: 123782180-123782638 | + | 160 | 459 | FGF2 | Proximal | 0 | Not documented | 86 |
| L1PA17_dup1242 | Uniquely | No | — | — | LINE | L1PA\|B\|x | chr4: 128769749-128770371 | + | 75 | 623 | HSPA4L | Distal | 7862 | Not documented | 87 |
| L1ME1_dup8744 | Multi | No | — | — | LINE | Other L1 | chr4: 132881728-132882174 | + | 130 | 447 | RP11-62N21.1 | Distal | 474784 | Not documented | 88 |
| L1M2c_dup89 | Uniquely | No | — | — | LINE | Other L1 | chr4: 13453697-13457563 | − | 100 | 3867 | RAB28 | Proximal | 0 | Not documented | 89 |
| MIR_dup42314 | Uniquely | No | — | — | SINE | MIR | chr4: 13485083-13485298 | − | 120 | 216 | RAB28 | Proximal | 0 | Not documented | 90 |
| AluSq2_dup13484 | Multi | No | — | — | SINE | Alu | chr4: 139088296-139088605 | − | 40 | 310 | SLC7A11 | Proximal | 0 | Not documented | 91 |
| L1MA4_dup2776 | Uniquely | No | — | — | LINE | Other L1 | chr4: 140334395-140335102 | − | 64 | 708 | NAA15 | Proximal | 0 | Not documented | 92 |
| AluSc_dup8819 | Multi | No | — | — | SINE | Alu | chr4: 140395049-140395356 | − | 38 | 308 | RAB33B | Proximal | 0 | Not documented | 93 |
| MLT1A0_dup5930 | Multi | No | — | — | LTR | ERVL- | chr4: 144113740-144113946 | − | 77 | 207 | USP38 | Proximal | 0 | Not documented | 94 |
| | | | | | | MaLR | | | | | | | | | |
| MER115_dup771 | Multi | No | — | — | DNA | DNA | chr4: 144114169-144114287 | + | 140 | 119 | USP38 | Proximal | 0 | Not documented | 95 |
| AluY_dup30685 | Multi | No | — | — | SINE | Alu | chr4: 148555880-148556172 | − | 39 | 293 | TMEM184C | Proximal | 0 | Not documented | 96 |
| L1MA9_dup4334 | Multi/ | No | — | — | LINE | Other L1 | chr4: 148556874-148558796 | + | 76 | 1923 | TMEM184C | Proximal | 0 | Not documented | 97 |
| | Uniquely | | | | | | | | | | | | | | |
| L1PB3_dup916 | Uniquely | No | — | — | LINE | L1PA\|B\|x | chr4: 151035618-151039083 | − | 97 | 3466 | DCLK2 | Proximal | 0 | Not documented | 98 |
| L1MB4_dup2331 | Uniquely | No | — | — | LINE | Other L1 | chr4: 151187747-151188929 | + | 79 | 1183 | LRBA | Proximal | 0 | Not documented | 99 |
| L2c_dup43282 | Uniquely | No | — | — | LINE | L2 | chr4: 155457045-155457402 | + | 150 | 358 | PLRG1 | Proximal | 0 | Not documented | 3400 |
| L3_dup11592 | Multi | No | — | — | LINE | CR1 | chr4: 15638047-15638153 | + | 140 | 107 | FBXL5 | Proximal | 0 | Not documented | 1 |
| Charlie9_dup279 | Multi | No | — | — | DNA | DNA | chr4: 15688122-15690764 | + | 59 | 2643 | FAM200B | Proximal | 0 | Not documented | 2 |
| L2c_dup43449 | Multi | No | — | — | LINE | L2 | chr4: 159600627-159600839 | − | 150 | 213 | ETFDH | Proximal | 0 | Not documented | 3 |
| AluSg_dup10545 | Multi | No | — | — | SINE | Alu | chr4: 159630398-159630699 | + | 39 | 302 | PPID | Proximal | 0 | Not documented | 4 |
| AluSx1_dup26566 | Multi | No | — | — | SINE | Alu | chr4: 166263509-166263808 | − | 50 | 300 | MSMO1 | Proximal | 0 | Not documented | 5 |
| MER11A_dup301 | Multi | No | — | — | LTR | ERVK | chr4: 166876975-166878039 | − | 25 | 1065 | TLL1 | Proximal | 0 | Not documented | 6 |
| L1M5_dup17716 | Multi | No | — | — | LINE | Other L1 | chr4: 167260374-167260745 | − | 130 | 372 | TLL1 | Distal | 235328 | Not documented | 7 |
| AluSp_dup12891 | Multi/ | No | — | — | SINE | Alu | chr4: 169771675-169771982 | − | 35 | 308 | PALLD | Proximal | 0 | Not documented | 8 |
| | Uniquely | | | | | | | | | | | | | | |
| L2b_dup28795 | Multi/ | No | — | — | LINE | L2 | chr4: 169772074-169772145 | + | 160 | 72 | PALLD | Proximal | 0 | Not documented | 9 |
| | Uniquely | | | | | | | | | | | | | | |
| AluSx3_dup7659 | Multi | No | — | — | SINE | Alu | chr4: 169826939-169827148 | − | 50 | 210 | CBR4 | Proximal | 0 | Not documented | 10 |
| L1PA7_dup3818 | Uniquely | No | — | — | LINE | L1PA\|B\|x | chr4: 169856136-169862185 | + | 44 | 6050 | CBR4 | Proximal | 0 | EVE annotated | 11 |
| L1MC1_dup4001 | Uniquely | No | — | — | LINE | Other L1 | chr4: 170473426-170475286 | + | 67 | 1861 | NEK1 | Proximal | | Not documented | 12 |
| AluJb_dup37178 | Multi | No | — | — | SINE | Alu | chr4: 170643010-170643268 | − | 69 | 259 | CLCN3 | Proximal | 0 | Not documented | 13 |
| AluSx3_dup7678 | Multi | No | — | — | SINE | Alu | chr4: 171763869-171764171 | + | 50 | 303 | AADAT | Distal | 751020 | Not documented | 14 |
| L1ME3B_dup2290 | Multi | No | — | — | LINE | Other L1 | chr4: 174284714-174286713 | + | 110 | 2000 | SAP30 | Distal | 4408 | Not documented | 15 |
| AluSx_dup35140 | Multi | No | — | — | SINE | Alu | chr4: 174290621-174290782 | + | 34 | 162 | SAP30 | Proximal | 339 | Not documented | 16 |
| MER5A_dup10112 | Multi | No | — | — | DNA | DNA | chr4: 174310726-174310858 | − | 72 | 133 | SCRG1 | Proximal | 0 | Not documented | 17 |
| MIRc_dup31270 | Uniquely | No | — | — | SINE | MIR | chr4: 174310861-174311064 | + | 150 | 204 | SCRG1 | Proximal | 0 | Not documented | 18 |
| X6A_LINE_dup87 | Uniquely | No | — | — | LINE | CR1 | chr4: 176733931-176733997 | − | 73 | 67 | GPM6A | Proximal | 0 | Not documented | 19 |
| L1MEc_dup5571 | Uniquely | No | — | — | LINE | Other L1 | chr4: 176775582-176776474 | + | 110 | 893 | GPM6A | Proximal | 0 | Not documented | 20 |
| L1MA4A_dup1805 | Multi | No | — | — | LINE | Other L1 | chr4: 176859261-176859427 | + | 71 | 16 | GPM6A | Proximal | 0 | Not documented | 21 |
| L1P3_dup946 | Multi | No | — | — | LINE | L1PA\|B\|x | chr4: 176859414-176859784 | − | 23 | 371 | GPM6A | Proximal | 0 | Not documented | 22 |
| L1PA7_dup3841 | Uniquely | No | — | — | LINE | L1PA\|B\|x | chr4: 176862554-176865304 | − | 34 | 2751 | GPM6A | Proximal | 0 | EVE annotated | 23 |
| L1MC2_dup1720 | Multi/ | No | — | — | LINE | Other L1 | chr4: 176887940-176890577 | + | 88 | 2638 | GPM6A | Proximal | 0 | Not documented | 24 |
| | Uniquely | | | | | | | | | | | | | | |
| L1ME1_dup9161 | Uniquely | No | — | — | LINE | Other L1 | chr4: 176902435-176905129 | − | 120 | 2695 | GPM6A | Proximal | 0 | Not documented | 3425 |
| Tigger1_dup3702 | Multi/ | No | — | — | DNA | DNA | chr4: 176915516-176916516 | − | 50 | 1001 | GPM6A | Proximal | 0 | Not documented | 26 |
| | Uniquely | | | | | | | | | | | | | | |
| AluSz6_dup11936 | Uniquely | No | — | — | SINE | Alu | chr4: 176919301-176919606 | − | 56 | 306 | GPM6A | Proximal | 0 | Not documented | 27 |
| AluSx_dup29935 | Multi | No | — | — | SINE | Alu | chr4: 1813536-1813663 | + | 40 | 128 | LETM1 | Proximal | 0 | Not documented | 28 |
| AluY_dup26034 | Multi | No | — | — | SINE | Alu | chr4: 1813664-1813942 | + | 39 | 279 | LETM1 | Proximal | 0 | Not documented | 29 |
| AluSx3_dup7802 | Uniquely | No | — | — | SINE | Alu | chr4: 186433245-186433534 | − | 49 | 290 | PDLIM3 | Proximal | 0 | Not documented | 30 |
| Tigger5_dup777 | Multi | No | — | — | DNA | DNA | chr4: 189660917-189660988 | + | 110 | 72 | TRIML1 | Distal | 592021 | Not documented | 31 |
| MER47C_dup236 | Multi | No | — | — | DNA | DNA | chr4: 189661003-189661086 | − | 92 | 84 | TRIML1 | Distal | 592107 | Not documented | 32 |
| LTR41B_dup301 | Multi | No | — | — | LTR | ERVL | chr4: 189661266-189661534 | − | 100 | 269 | TRIML1 | Distal | 592370 | Not documented | 33 |
| MLT2D_dup1415 | Multi | No | — | — | LTR | ERVL | chr4: 189661660-189661973 | − | 120 | 314 | TRIML1 | Distal | 592764 | Not documented | 34 |
| MER20_dup3879 | Uniquely | No | — | — | DNA | DNA | chr4: 25124851-25125051 | − | 68 | 201 | SEPSECS | Proximal | 0 | Not documented | 35 |
| AluSz_dup21691 | Multi | No | — | — | SINE | Alu | chr4: 39552527-39552829 | − | 46 | 303 | SMIM14 | Proximal | 0 | Not documented | 36 |
| FLAM_A_dup3850 | Multi | No | — | — | SINE | Alu | chr4: 4237871-4238001 | + | 62 | 131 | TMEM128 | Proximal | 0 | Not documented | 37 |
| AluSp_dup10887 | Multi | No | — | — | SINE | Alu | chr4: 434448-434754 | − | 49 | 307 | ZNF721 | Proximal | 0 | Not documented | 38 |
| L2b_dup24080 | Multi | No | — | — | LINE | L2 | chr4: 4461207-4461416 | − | 160 | 210 | STX18 | Proximal | 0 | Not documented | 39 |
| L2_dup14066 | Multi | No | — | — | LINE | L2 | chr4: 45332652-45333173 | − | 140 | 522 | GNPDA2 | Distal | 604041 | Not documented | 40 |
| L1PA6_dup1452 | Multi | No | — | — | LINE | L1PA\|B\|x | chr4: 46726622-46727888 | − | 37 | 1267 | COX7B2 | Distal | 8957 | Not documented | 41 |
| L1HS_dup376 | Multi | No | — | — | LINE | L1PA\|B\|x | chr4: 46798328-46799122 | + | 1.8 | 795 | COX7B2 | Proximal | 0 | EVE annotated | 42 |
| L1ME1_dup7598 | Uniquely | No | — | — | LINE | Other L1 | chr4: 4740714-4741673 | + | 100 | 960 | MSX1 | Distal | 119721 | Not documented | 43 |
| MIR3_dup23027 | Multi/ | No | — | — | SINE | MIR | chr4: 4865049-4865196 | − | 130 | 148 | MSX1 | Proximal | 0 | Not documented | 44 |
| | Uniquely | | | | | | | | | | | | | | |
| CER_dup10 | Multi | No | — | — | Other | Other | chr4: 49198848-49202172 | − | 47 | 3325 | CWH43 | Distal | 134751 | Not documented | 45 |
| | | | | | | Repeats | | | | | | | | | |
| CER_dup11 | Multi | No | — | — | Other | Other | chr4: 49594334-49601946 | + | 49 | 7613 | CWH43 | Distal | 530237 | Not documented | 46 |
| | | | | | | Repeats | | | | | | | | | |
| MIRb_dup59856 | Multi | No | — | — | SINE | MIR | chr4: 52889197-52889470 | + | 120 | 274 | SGCB | Proximal | 0 | Not documented | 47 |
| MIR_dup45248 | Multi | No | — | — | SINE | MIR | chr4: 53579898-53579929 | − | 110 | 32 | ERVMER34-1 | Distal | 8857 | Not documented | 48 |
| LTR21A_dup22 | Multi | No | — | — | LTR | ERV1 | chr4: 53579930-53580342 | + | 96 | 413 | ERVMER34-1 | Distal | 8444 | Not documented | 49 |
| L2a_dup44366 | Multi | No | — | — | LINE | L2 | chr4: 57326029-57326059 | + | 140 | 31 | PAICS | Proximal | 0 | Not documented | 3450 |
| AluJb_dup33570 | Multi/ | No | — | — | SINE | Alu | chr4: 57326060-57326349 | − | 88 | 290 | PAICS | Proximal | 0 | Not documented | 51 |
| | Uniquely | | | | | | | | | | | | | | |
| L2a_dup44367 | Multi | No | — | — | LINE | L2 | chr4: 57326350-57326596 | + | 140 | 247 | PAICS | Proximal | 0 | Not documented | 52 |
| Charlie4z_dup1561 | Multi | No | — | — | DNA | DNA | chr4: 57326705-57326844 | − | 120 | 140 | PAICS | Proximal | 0 | Not documented | 53 |
| AluSx1_dup24171 | Multi/ | No | — | — | SINE | Alu | chr4: 57326856-57327154 | + | 58 | 299 | PAICS | Proximal | 0 | Not documented | 54 |
| | Uniquely | | | | | | | | | | | | | | |
| MIR3_dup24485 | Uniquely | No | — | — | SINE | MIR | chr4: 57523903-57524017 | − | 160 | 115 | HOPX | Proximal | 0 | Not documented | 55 |
| THE1B-int_dup1110 | Uniquely | No | — | — | LTR | ERVL- | chr4: 62188050-62189600 | − | 47 | 1551 | LPHN3 | Proximal | 0 | Not documented | 56 |
| | | | | | | MaLR | | | | | | | | | |
| Tigger1_dup3073 | Multi | No | — | — | DNA | DNA | chr4: 62216180-62217970 | + | 64 | 1791 | LPHN3 | Proximal | 0 | Not documented | 57 |
| FLAM_A_dup3862 | Multi | No | — | — | SINE | Alu | chr4: 6718785-6718898 | − | 68 | 114 | BLOC154 | Proximal | 0 | Not documented | 58 |
| L1PA16_dup3061 | Uniquely | No | — | — | LINE | L1PA\|B\|x | chr4: 6820192-6821998 | + | 58 | 1807 | KIAA0232 | Proximal | 0 | Not documented | 59 |
| MER41B_dup630 | Uniquely | No | — | — | LTR | ERV1 | chr4: 68587380-68588008 | − | 44 | 629 | GNRHR | Dista | 17039 | Not documented | 60 |
| L4_dup4369 | Multi | No | — | — | LINE | RTE | chr4: 69698174-69698216 | + | 74 | 43 | UGT2B10 | Proximal | 1261 | Not documented | 61 |
| MSTA_dup4794 | Multi | No | — | — | LTR | ERVL- | chr4: 70040876-70041168 | − | 91 | 293 | UGT2B11 | Distal | 24502 | Not documented | 62 |
| | | | | | | MaLR | | | | | | | | | |
| L1PA5_dup2724 | Multi | No | — | — | LINE | L1PA\|B\|x | chr4: 70041769-70043973 | + | 32 | 2205 | UGT2B11 | Distal | 21697 | Not documented | 63 |
| HERV30-int_dup13 | Multi | No | — | — | LTR | ERV1 | chr4: 70136781-70137681 | + | 54 | 901 | UGT2B28 | Distal | 8537 | Not documented | 64 |
| L1MA10_dup1127 | Multi | No | — | — | LINE | Other L1 | chr4: 70236575-70237009 | + | 90 | 435 | UGT2B28 | Distal | 75808 | Not documented | 65 |
| HERV30-int_dup15 | Multi | No | — | — | LTR | ERV1 | chr4: 70245761-70246661 | + | 49 | 901 | UGT2B28 | Distal | 84994 | Not documented | 66 |
| LTR16A_dup1835 | Multi | No | — | — | LTR | ERVL | chr4: 70252189-70252482 | − | 160 | 294 | UGT2B28 | Distal | 91422 | Not documented | 67 |
| MER57-int_dup353 | Multi | No | — | — | LTR | ERV1 | chr4: 70331213-70331838 | + | 76 | 626 | UGT2B4 | Distal | 14046 | Not documented | 68 |
| MER52D_dup184 | Multi | No | — | — | LTR | ERV1 | chr4: 70386011-70387496 | + | 110 | 1486 | UGT2B4 | Proximal | 0 | Not documented | 69 |
| LTR12C_dup702 | Multi | No | — | — | LTR | ERV1 | chr4: 70440959-70442539 | − | 66 | 1581 | UGT2A2 | Distal | 11597 | Not documented | 70 |
| L1PBa_dup565 | Multi | No | — | — | LINE | L1PA\|B\|x | chr4: 70524197-70526436 | + | 75 | 2240 | UGT2A1 | Distal | 5233 | Not documented | 71 |
| HERV9-int_dup260 | Multi | No | — | — | LTR | ERV1 | chr4: 70691144-70693912 | + | 34 | 2769 | SULT1E1 | Proximal | 0 | EVE annotated | 72 |
| L1MC2_dup1493 | Multi | No | — | — | LINE | Other L1 | chr4: 70755549-70756508 | + | 84 | 960 | SULT1E1 | Distal | 29680 | Not documented | 73 |
| MLT1E1_dup280 | Multi | No | — | — | LTR | ERVL- | chr4: 71232234-71232290 | − | 81 | 57 | SMR3A | Proximal | 0 | Not documented | 74 |
| | | | | | | MaLR | | | | | | | | | |
| MLT1E_dup205 | Multi | No | — | — | LTR | ERVL- | chr4: 71255254-71255310 | − | 65 | 57 | SMR3B | Proximal | 0 | Not documented | 3475 |
| | | | | | | MaLR | | | | | | | | | |
| L2a_dup45216 | Multi | No | — | — | LINE | L2 | chr4: 71629319-71629375 | − | 84 | 57 | RUFY3 | Proximal | 0 | Not documented | 76 |
| AluJo_dup16283 | Uniquely | No | — | — | SINE | Alu | chr4: 71663812-71664106 | + | 66 | 295 | RUFY3 | Proximal | 0 | Not documented | 77 |
| AluSx1_dup24430 | Multi | No | — | — | SINE | Alu | chr4: 71664586-71664891 | + | 45 | 306 | RUFY3 | Proximal | 0 | Not documented | 78 |
| MLT1J1-int_dup158 | Uniquely | No | — | — | LTR | ERVL- | chr4: 74127529-74128787 | + | 140 | 1259 | ANKRD17 | Dista | 3015 | Not documented | 79 |
| | | | | | | MaLR | | | | | | | | | |
| L1PA7_dup3264 | Uniquely | No | — | — | LINE | L1PA\|B\|x | chr4: 77398829-77405306 | + | 36 | 6478 | SHROOM3 | Proximal | 0 | EVE annotated | 80 |
| AluSz_dup22678 | Multi | No | — | — | SINE | Alu | chr4: 77958233-77958527 | + | 59 | 295 | sept-11 | Proximal | 0 | Not documented | 81 |
| AluSq2_dup12853 | Multi/ | No | — | — | SINE | Alu | chr4: 83707671-83707967 | + | 49 | 297 | SCD5 | Proximal | 0 | Not documented | 82 |
| | Uniquely | | | | | | | | | | | | | | |
| HSMAR2_dup425 | Multi | No | — | — | DNA | DNA | chr4: 83814862-83816115 | − | 50 | 1254 | SEC31A | Proximal | 0 | Not documented | 83 |
| AluSq2_dup12915 | Multi | No | — | — | SINE | Alu | chr4: 87359561-87359860 | + | 50 | 300 | MAPK10 | Proximal | 0 | Not documented | 84 |
| AluSx1_dup22571 | Multi | No | — | — | SINE | Alu | chr4: 950232-950531 | − | 44 | 300 | TMEM175 | Proximal | 0 | Not documented | 85 |
| AluSx_dup33047 | Multi | No | — | — | SINE | Alu | chr4: 95585739-95586030 | + | 42 | 292 | PDLIM5 | Proximal | 0 | Not documented | 86 |
| AluSc_dup8454 | Multi | No | — | — | SINE | Alu | chr4: 95586353-95586657 | + | 43 | 305 | PDLIM5 | Proximal | 0 | Not documented | 87 |
| MER2_dup2442 | Multi | No | — | — | DNA | DNA | chr4: 95588452-95588786 | + | 64 | 335 | PDLIM5 | Proximal | 0 | Not documented | 88 |
| L1MB2_dup2017 | Multi | No | — | — | LINE | Other L1 | chr4: 99812474-99812953 | + | 80 | 480 | EIF4E | Proximal | 0 | Not documented | 89 |
| L1ME3C_dup3233 | Multi | No | — | — | LINE | Other L1 | chr4: 99992136-99992861 | + | 100 | 726 | ADH5 | Proximal | 0 | Not documented | 90 |
| L2c_dup40961 | Multi | No | — | — | LINE | L2 | chr4: 99992986-99993090 | − | 140 | 105 | ADH5 | Proximal | 0 | Not documented | 91 |
| AluSz_dup25269 | Uniquely | No | — | — | SINE | Alu | chr5: 10279657-10279951 | − | 37 | 295 | CMBL | Proximal | 0 | Not documented | 92 |
| AluSx_dup36018 | Multi | No | — | — | SINE | Alu | chr5: 10435811-10436129 | − | 50 | 319 | MARCH6 | Proximal | 0 | Not documented | 93 |
| AluSq2_dup15651 | Multi | No | — | — | SINE | Alu | chr5: 108679515-108679821 | + | 40 | 307 | PJA2 | Proximal | 0 | Not documented | 94 |
| Charlie7_dup1334 | Multi | No | — | — | DNA | DNA | chr5: 108718863-108719345 | + | 130 | 483 | PJA2 | Proximal | 0 | Not documented | 95 |
| L1ME1_dup10367 | Multi | No | — | — | LINE | Other L1 | chr5: 108737589-108739491 | + | 110 | 1903 | PJA2 | Proximal | 0 | Not documented | 96 |
| L1M4c_dup1586 | Multi | No | — | — | LINE | Other L1 | chr5: 11029278-11031636 | − | 73 | 2359 | CTNND2 | Proximal | 0 | Not documented | 97 |
| L3b_dup2417 | Multi | No | — | — | LINE | CR1 | chr5: 110464277-110464336 | + | 110 | 60 | WDR36 | Proximal | 0 | Not documented | 98 |
| L1M5_dup20574 | Multi/ | No | — | — | LINE | Other L1 | chr5: 110464832-110465421 | − | 120 | 590 | WDR36 | Proximal | 0 | Not documented | 99 |
| | Uniquely | | | | | | | | | | | | | | |
| L2a_dup52864 | Uniquely | No | — | — | LINE | L2 | chr5: 11077246-11078295 | − | 130 | 1050 | CTNND2 | Proximal | 0 | Not documented | 3500 |
| L1MEc_dup5776 | Uniquely | No | — | — | LINE | Other L1 | chr5: 11090915-11092389 | − | 95 | 1475 | CTNND2 | Proximal | 0 | Not documented | 1 |
| MLT1E2-int_dup39 | Uniquely | No | — | — | LTR | ERVL- | chr5: 11127676-11128055 | + | 110 | 380 | CTNND2 | Proximal | 0 | Not documented | 2 |
| | | | | | | MaLR | | | | | | | | | |
| MLT1E2_dup1163 | Uniquely | No | — | — | LTR | ERVL- | chr5: 11134013-11134632 | + | 120 | 620 | CTNND2 | Proximal | 0 | Not documented | 3 |
| | | | | | | MaLR | | | | | | | | | |
| MSTD_dup2423 | Uniquely | No | — | — | LTR | ERVL- | chr5: 11141918-11142295 | + | 72 | 378 | CTNND2 | Proximal | 0 | Not documented | 4 |
| | | | | | | MaLR | | | | | | | | | |
| MLT1C_dup5475 | Uniquely | No | — | — | LTR | ERVL- | chr5: 11143388-11143836 | − | 80 | 449 | CTNND2 | Proximal | 0 | Not documented | 5 |
| | | | | | | MaLR | | | | | | | | | |
| MIRb_dup72728 | Multi | No | — | — | SINE | MIR | chr5: 112105335-112105494 | + | 160 | 160 | APC | Proximal | 0 | Not documented | 6 |
| X5A_LINE_dup57 | Multi | No | — | — | LINE | CR1 | chr5: 112177303-112177355 | − | 80 | 53 | CTC-554D6.1 | Proximal | 0 | Not documented | 7 |
| AluSx1_dup30100 | Multi | No | — | — | SINE | Alu | chr5: 112361043-112361307 | + | 41 | 265 | MCC | Proximal | 0 | Not documented | 8 |
| AluSg_dup12048 | Uniquely | No | — | — | SINE | Alu | chr5: 112436162-112436467 | − | 48 | 306 | MCC | Proximal | 0 | Not documented | 9 |
| LTR54B_dup246 | Uniquely | No | — | — | LTR | ERV1 | chr5: 11262190-11262592 | + | 90 | 403 | CTNND2 | Proximal | 0 | Not documented | 10 |
| AluY_dup32498 | Multi | No | — | — | SINE | Alu | chr5: 11262593-11262903 | + | 28 | 311 | CTNND2 | Proximal | 0 | Not documented | 11 |
| AluSg_dup11014 | Multi | No | — | — | SINE | AIL | chr5: 11283506-11283815 | + | 45 | 310 | CTNND2 | Proximal | 0 | Not documented | 12 |
| L1PA10_dup2034 | Uniquely | No | — | — | LINE | L1PA\|B\|x | chr5: 11284378-11284914 | − | 40 | 537 | CTNND2 | Proximal | 0 | Not documented | 13 |
| L1MC3_dup4223 | Uniquely | No | — | — | LINE | Other L1 | chr5: 113764359-113768196 | − | 75 | 3838 | KCNN2 | Proximal | 0 | Not documented | 14 |
| L2a_dup52897 | Uniquely | No | — | — | LINE | L2 | chr5: 11393924-11394471 | + | 120 | 548 | CTNND2 | Proximal | 0 | Not documented | 15 |
| L1MC4a_dup8635 | Multi | No | — | — | LINE | Other L1 | chr5: 114877166-114877462 | + | 120 | 297 | FEM1C | Proximal | 0 | Not documented | 16 |
| AluJr4_dup5614 | Multi | No | — | — | SINE | Alu | chr5: 115166243-115166513 | − | 99 | 271 | ATG12 | Proximal | 0 | Not documented | 17 |
| HAL1_dup8383 | Uniquely | No | — | — | LINE | Other L1 | chr5: 11570151-11571896 | − | 130 | 1746 | CTNND2 | Proximal | 0 | Not documented | 18 |
| L2_dup19390 | Multi | No | — | — | LINE | L2 | chr5: 115831980-115832027 | + | 150 | 48 | SEMA6A | Proximal | 0 | Not documented | 19 |
| L2a_dup58248 | Uniquely | No | — | — | LINE | L2 | chr5: 115904675-115905678 | − | 120 | 1004 | SEMA6A | Proximal | 0 | Not documented | 20 |
| L1PA15_dup2343 | Uniquely | No | — | — | LINE | L1PA\|B\|x | chr5: 11751343-11756192 | − | 67 | 4850 | CTNND2 | Proximal | 0 | Not documented | 21 |
| HERVH-int_dup2235 | Uniquely | No | — | — | LTR | ERV1 | chr5: 118284314-118285623 | − | 61 | 1310 | DTWD2 | Proximal | 0 | EVE annotated | 22 |
| MamRep137_dup792 | Multi | No | — | — | DNA | DNA | chr5: 122344032-122344421 | + | 120 | 390 | SNX24 | Proximal | 0 | Not documented | 23 |
| MIRb_dup73377 | Uniquely | No | — | — | SINE | MIR | chr5: 124043741-124043850 | − | 160 | 110 | ZNF608 | Proximal | 0 | Not documented | 24 |
| MIRb_dup73378 | Multi/ | No | — | — | SINE | MIR | chr5: 124043948-124044097 | − | 120 | 150 | ZNF608 | Proximal | 0 | Not documented | 3525 |
| | Uniquely | | | | | | | | | | | | | | |
| AluSx_dup39421 | Multi/ | No | — | — | SINE | Alu | chr5: 124044583-124044891 | + | 40 | 309 | ZNF608 | Proximal | 0 | Not documented | 26 |
| | Uniquely | | | | | | | | | | | | | | |
| HAL1_dup9468 | Multi/ | No | — | — | LINE | Other L1 | chr5: 125879104-125879355 | − | 130 | 252 | ALDH7A1 | Proximal | 0 | Not documented | 27 |
| | Uniquely | | | | | | | | | | | | | | |
| AluSx1_dup30361 | Multi/ | No | — | — | SINE | Alu | chr5: 125879657-125879950 | − | 59 | 294 | ALDH7A1 | Proximal | 0 | Not documented | 28 |
| | Uniquely | | | | | | | | | | | | | | |
| AluSp_dup14573 | Multi/ | No | — | — | SINE | Alu | chr5: 125930131-125930435 | − | 27 | 305 | ALDH7A1 | Proximal | 0 | Not documented | 29 |
| | Uniquely | | | | | | | | | | | | | | |
| AluSg_dup12210 | Multi | No | — | — | SINE | Alu | chr5: 130536730-130537035 | + | 40 | 306 | LYRM7 | Proximal | 0 | Not documented | 30 |
| LTR12_dup267 | Uniquely | No | — | — | LTR | ERV1 | chr5: 130822718-130823426 | − | 40 | 709 | RAPGEF6 | Proximal | 0 | Not documented | 31 |
| MIRc_dup34669 | Uniquely | No | — | — | SINE | MIR | chr5: 131807294-131807486 | + | 110 | 193 | C5orf56 | Proximal | 0 | Not documented | 32 |
| AluSq2_dup15900 | Multi | No | — | — | SINE | Alu | chr5: 131818356-131818633 | − | 34 | 278 | IRF1 | Proximal | 0 | Not documented | 33 |
| MER94_dup1749 | Multi | No | — | — | DNA | DNA | chr5: 132091961-132092088 | + | 100 | 128 | sept-08 | Proximal | 0 | Not documented | 34 |
| Arthur1A_dup418 | Multi/ | No | — | — | DNA | DNA | chr5: 132092787-132092938 | + | 90 | 152 | sept-08 | Proximal | 0 | Not documented | 35 |
| | Uniquely | | | | | | | | | | | | | | |
| AluY_dup36339 | Multi | No | — | — | SINE | Alu | chr5: 132158044-132158353 | − | 16 | 310 | SHROOM1 | Proximal | 0 | Not documented | 36 |
| MIRb_dup73853 | Multi | No | — | — | SINE | MIR | chr5: 132202799-132202951 | − | 140 | 153 | UQCRQ | Proximal | 0 | Not documented | 37 |
| L1MB7_dup6868 | Multi | No | — | — | LINE | Other L1 | chr5: 132203035-132203097 | + | 87 | 63 | UQCRQ | Proximal | 0 | Not documented | 38 |
| AluJo_dup20196 | Multi | No | — | — | SINE | Alu | chr5: 133738228-133738341 | + | 88 | 114 | CDKN2AIPNL | Proximal | 0 | Not documented | 39 |
| AluSz_dup28055 | Multi | Nc | — | — | SINE | Alu | chr5: 133937455-133937751 | + | 57 | 297 | SAR1B | Proximal | 0 | Not documented | 40 |
| AluSx_dup39889 | Multi | No | — | — | SINE | Alu | chr5: 133938287-133938598 | + | 58 | 312 | SAR1B | Proximal | 0 | Not documented | 41 |
| AluJb_dup42378 | Multi/ | No | — | — | SINE | Alu | chr5: 137805158-137805351 | − | 82 | 194 | EGR1 | Proximal | 155 | Not documented | 42 |
| | Uniquely | | | | | | | | | | | | | | |
| MER77B_dup440 | Multi | No | — | — | LTR | ERVL | chr5: 137805452-137805488 | − | 74 | 37 | EGR1 | Proximal | 449 | Not documented | 43 |
| L2c_dup49711 | Multi | No | — | — | LINE | L2 | chr5: 137806011-137806181 | − | 130 | 171 | EGR1 | Proximal | 1008 | Not documented | 44 |
| MLT1L_dup4195 | Multi | No | — | — | LTR | ERVL- | chr5: 137890535-137890700 | + | 120 | 166 | HSPA9 | Proximal | 0 | Not documented | 45 |
| | | | | | | MaLR | | | | | | | | | |
| Charlie7a_dup537 | Multi/ | No | — | — | DNA | DNA | chr5: 139223883-139224080 | − | 69 | 198 | PSD2 | Proximal | 0 | Not documented | 46 |
| | Uniquely | | | | | | | | | | | | | | |
| L1PB1_dup3978 | Uniquely | No | — | — | LINE | L1PA\|B\|x | chr5: 13967758-13972974 | − | 41 | 5217 | DNAH5 | Distal | 23107 | EVE annotated | 47 |
| AluJb_dup42703 | Multi | No | — | — | SINE | Alu | chr5: 140564974-140565273 | − | 76 | 300 | PCDHB16 | Proximal | 0 | Not documented | 48 |
| AluSc_dup10676 | Multi | No | — | — | SINE | Alu | chr5: 140569740-140570047 | − | 38 | 308 | PCDHB10 | Proximal | 1896 | Not documented | 49 |
| L1MC5_dup6557 | Multi | No | — | — | LINE | Other L1 | chr5: 140605692-140605736 | + | 31 | 45 | PCDHB14 | Proximal | 0 | Not documented | 3550 |
| AluSp_dup14948 | Multi | No | — | — | SINE | Alu | chr5: 140761361-140761694 | − | 30 | 334 | PCDHGA6 | Proximal | 0 | Not documented | 51 |
| AluJr_dup21879 | Multi/ | No | — | — | SINE | Alu | chr5: 140859717-140859992 | + | 76 | 276 | PCDHGC3 | Proximal | 0 | Not documented | 52 |
| | Uniquely | | | | | | | | | | | | | | |
| AluY_dup36782 | Uniquely | No | — | — | SINE | Alu | chr5: 140879752-140880075 | − | 25 | 324 | PCDHGC5 | Proximal | 0 | Not documented | 53 |
| L3_dup14035 | Uniquely | No | — | — | LINE | CR1 | chr5: 14146591-14146873 | + | 140 | 283 | TRIO | Proximal | 0 | Not documented | 54 |
| Tigger8_dup323 | Multi/ | No | — | — | DNA | DNA | chr5: 141532481-141533048 | − | 120 | 568 | NDFIP1 | Proximal | 0 | Not documented | 55 |
| | Uniquely | | | | | | | | | | | | | | |
| MIRb_dup74878 | Multi | No | — | — | SINE | MIR | chr5: 141690510-141690612 | + | 130 | 103 | SPRY4 | Proximal | 0 | Not documented | 56 |
| AluSx_dup36120 | Uniquely | No | — | — | SINE | Alu | chr5: 14173300-14173620 | − | 58 | 321 | TRIO | Proximal | 0 | Not documented | 57 |
| L1MC4_dup8534 | Uniquely | No | — | — | LINE | Other L1 | chr5: 14194892-14195959 | − | 110 | 1068 | TRIO | Proximal | 0 | Not documented | 58 |
| L2b_dup32774 | Multi/ | No | — | — | LINE | L2 | chr5: 141972535-141972596 | + | 120 | 62 | FGF1 | Proximal | 0 | Not documented | 59 |
| | Uniquely | | | | | | | | | | | | | | |
| MER5A1_dup5344 | Multi/ | No | — | — | DNA | DNA | chr5: 141973758-141973926 | + | 110 | 169 | FGF1 | Proximal | 0 | Not documented | 60 |
| | Uniquely | | | | | | | | | | | | | | |
| LTR40a_dup636 | Uniquely | No | — | — | LTR | ERVL | chr5: 14197697-14198197 | − | 93 | 501 | TRIO | Proximal | 0 | Not documented | 61 |
| MIR3_dup27356 | Uniquely | No | — | — | SINE | MIR | chr5: 14198502-14198575 | − | 120 | 74 | TRIO | Proximal | 0 | Not documented | 62 |
| AluSx1_dup27645 | Multi | No | — | — | SINE | Alu | chr5: 14199065-14199328 | + | 55 | 264 | TRIO | Proximal | 0 | Not documented | 63 |
| L2b_dup29528 | Uniquely | No | — | — | LINE | L2 | chr5: 14200354-14200672 | + | 140 | 319 | TRIO | Proximal | 0 | Not documented | 64 |
| AluSc_dup10724 | Multi | No | — | — | SINE | Alu | chr5: 142713053-142713365 | + | 40 | 313 | NR3C1 | Proximal | 0 | Not documented | 65 |
| AluY_dup36914 | Multi | No | — | — | SINE | Alu | chr5: 143539271-143539571 | − | 26 | 301 | YIPF5 | Proximal | 0 | Not documented | 66 |
| MER5A_dup10367 | Multi | Nc | — | — | DNA | DNA | chr5: 14482379-14482415 | − | 61 | 37 | TRIO | Proximal | 0 | Not documented | 67 |
| L2a_dup53071 | Uniquely | No | — | — | LINE | L2 | chr5: 14483249-14483387 | − | 140 | 139 | TRIO | Proximal | 0 | Not documented | 68 |
| THE1A-int_dup571 | Uniquely | No | — | — | LTR | ERVL- | chr5: 146108493-146110046 | + | 41 | 1554 | PPP2R2B | Proximal | 0 | Not documented | 69 |
| | | | | | | MaLR | | | | | | | | | |
| L2b_dup33009 | Uniquely | No | — | — | LINE | L2 | chr5: 146233378-146233696 | + | 160 | 319 | PPP2R2B | Proximal | 0 | Not documented | 70 |
| L1MDa_dup2166 | Uniquely | No | — | — | LINE | Other L1 | chr5: 146361639-146362739 | − | 75 | 1101 | PPP2R2B | Proximal | 0 | Not documented | 71 |
| L1MA7_dup2695 | Uniquely | No | — | — | LINE | Other L1 | chr5: 146363512-146364571 | − | 75 | 1060 | PPP2R2B | Proximal | 0 | Not documented | 72 |
| L1PA8_dup2768 | Uniquely | No | — | — | LINE | L1PA\|B\|x | chr5: 147036874-147039060 | − | 74 | 2187 | JAKMIP2 | Proximal | 0 | Not documented | 73 |
| AluYc_dup2686 | Uniquely | No | — | — | SINE | Alu | chr5: 148726289-148726587 | − | 10 | 299 | GRPEL2 | Proximal | 0 | Not documented | 74 |
| MIRc_dup35587 | Multi | No | — | — | SINE | MIR | chr5: 150175269-150175492 | + | 110 | 224 | AC010441.1 | Proximal | 0 | Not documented | 3575 |
| MER3_dup3701 | Multi/ | No | — | — | DNA | DNA | chr5: 151041243-151041447 | + | 98 | 205 | SPARC | Proximal | 0 | Not documented | 76 |
| | Uniquely | | | | | | | | | | | | | | |
| L2a_dup60826 | Multi/ | No | — | — | LINE | L2 | chr5: 151041507-151041675 | + | 140 | 169 | SPARC | Proximal | 0 | Not documented | 77 |
| | Uniquely | | | | | | | | | | | | | | |
| X8_LINE_dup104 | Multi | No | — | — | LINE | CR1 | chr5: 154255650-154255706 | + | 100 | 57 | CNOT8 | Proximal | 0 | Not documented | 78 |
| L2c_dup44889 | Uniquely | No | — | — | LINE | L2 | chr5: 15576905-15577054 | + | 140 | 150 | FBXL7 | Proximal | 0 | Not documented | 79 |
| LTR5_Hs_dup207 | Multi | No | — | — | LTR | ERVK | chr5: 156092929-156093896 | − | 4.1 | 968 | SGCD | Proximal | 0 | EVE annotated | 80 |
| AluSz_dup28813 | Uniquely | No | — | — | SINE | Alu | chr5: 156323837-156324161 | + | 48 | 325 | TIMD4 | Distal | 22133 | Not documented | 81 |
| AluSc_dup10854 | Uniquely | No | — | — | SINE | Alu | chr5: 156402621-156402912 | − | 42 | 292 | TIMD4 | Distal | 12356 | Not documented | 82 |
| AluSx_dup36189 | Multi | No | — | — | SINE | Alu | chr5: 15709367-15709665 | + | 50 | 299 | FBXL7 | Proximal | 0 | Not documented | 83 |
| FAM_dup1753 | Multi | No | — | — | SINE | Alu | chr5: 159829407-159829542 | + | 90 | 136 | SLU7 | Proximal | 0 | Not documented | 84 |
| AluJo_dup18275 | Multi/ | No | — | — | SINE | Alu | chr5: 1629202-1629272 | + | 64 | 71 | LPCAT1 | Distal | 105111 | Not documented | 85 |
| | Uniquely | | | | | | | | | | | | | | |
| AluJb_dup37858 | Multi | No | — | — | SINE | Alu | chr5: 1629514-1629717 | − | 74 | 204 | LPCAT1 | Distal | 105423 | Not documented | 86 |
| L1MC1_dup4948 | Multi | No | — | — | LINE | Other L1 | chr5: 170310273-170311239 | − | 60 | 967 | RANBP17 | Proximal | 0 | Not documented | 87 |
| MIRb_dup79266 | Uniquely | No | — | — | SINE | MIR | chr5: 171762421-171762621 | + | 150 | 201 | SH3PXD2B | Proximal | 0 | Not documented | 88 |
| AluSg4_dup2333 | Multi | No | — | — | SINE | Alu | chr5: 174306184-174306489 | − | 46 | 306 | MSX2 | Distal | 148289 | Not documented | 89 |
| HUERS-P1- | Multi | No | — | — | LTR | ERV1 | chr5: 174319008-174320162 | + | 60 | 1155 | MSX2 | Distal | 161113 | Not documented | 90 |
| int_dup183 | | | | | | | | | | | | | | | |
| AluYc3_dup182 | Multi | No | — | — | SINE | Alu | chr5: 174954919-174955211 | − | 19 | 293 | SFXN1 | Proximal | 0 | Not documented | 91 |
| MER2_dup3306 | Multi/ | No | — | — | DNA | DNA | chr5: 174955212-174955483 | + | 80 | 272 | SFXN1 | Proximal | 0 | Not documented | 92 |
| | Uniquely | | | | | | | | | | | | | | |
| AluSx1_dup32247 | Multi | No | — | — | SINE | Alu | chr5: 175774195-175774502 | − | 52 | 308 | KIAA1191 | Proximal | 0 | Not documented | 93 |
| MIR_dup63091 | Multi | No | — | — | SINE | MIR | chr5: 176733741-176733813 | − | 110 | 73 | PRELID1 | Proximal | 0 | Not documented | 94 |
| L2a_dup62947 | Multi | No | — | — | LINE | L2 | chr5: 176733849-176733947 | + | 74 | 99 | PRELID1 | Proximal | 0 | Not documented | 95 |
| L3_dup16510 | Multi | No | — | — | LINE | CR1 | chr5: 176942628-176942828 | − | 150 | 201 | DDX41 | Proximal | 0 | Not documented | 96 |
| HAL1-2a_MD_dup356 | Multi | No | — | — | LINE | Other L1 | chr5: 179132658-179132812 | − | 120 | 155 | CANX | Proximal | 0 | Not documented | 97 |
| L2_dup20431 | Multi | No | — | — | LINE | L2 | chr5: 179156477-179156554 | + | 150 | 78 | CANX | Proximal | 0 | Not documented | 98 |
| MER61B_dup151 | Multi | No | — | — | LTR | ERV1 | chr5: 179238359-179238651 | + | 80 | 293 | SQSTM1 | Proximal | 0 | Not documented | 99 |
| MER61-int_dup409 | Multi | No | — | — | LTR | ERV1 | chr5: 179238653-179238836 | + | 73 | 184 | SQSTM1 | Proximal | 0 | Not documented | 3600 |
| MER67A_dup186 | Multi | No | — | — | LTR | ERV1 | chr5: 180258010-180258181 | − | 130 | 172 | MGAT1 | Distal | 15359 | Not documented | 1 |
| AluSq2_dup16922 | Multi | No | — | — | SINE | Alu | chr5: 180258416-180258530 | − | 47 | 115 | MGAT1 | Distal | 15765 | Not documented | 2 |
| FLAM_C_dup6874 | Multi | No | — | — | SINE | Alu | chr5: 180621260-180621390 | + | 76 | 131 | TRIM7 | Proximal | 0 | Not documented | 3 |
| AluJr_dup22800 | Multi | No | — | — | SINE | Alu | chr5: 180664118-180664259 | − | 93 | 142 | GNB2L1 | Proximal | 0 | Not documented | 4 |
| MER112_dup1653 | Multi/ | No | — | — | DNA | DNA | chr5: 180690484-180690680 | − | 120 | 197 | TRIM52 | Distal | 2366 | Not documented | 5 |
| | Uniquely | | | | | | | | | | | | | | |
| L1HS_dup516 | Multi | No | — | — | LINE | L1PA\|B\|x | chr5: 24370616-24371578 | − | 23 | 963 | CDH10 | Distal | 115632 | EVE annotated | 6 |
| MER63A_dup1085 | Uniquely | No | — | — | DNA | DNA | chr5: 24571842-24572038 | − | 100 | 197 | CDH10 | Proximal | 0 | Not documented | 7 |
| AluJb_dup37826 | Multi | No | — | — | SINE | Alu | chr5: 276155-276358 | + | 71 | 204 | PDCD6 | Proximal | 0 | Not documented | 8 |
| HERVK-int_dup75 | Multi | No | — | — | LTR | ERVK | chr5: 30487720-30495254 | − | 7.3 | 7535 | CDH6 | Distal | 698604 | EVE annotated | 9 |
| L2c_dup45214 | Multi | No | — | — | LINE | L2 | chr5: 31530775-31531061 | + | 140 | 287 | DROSHA | Proximal | 0 | Not documented | 10 |
| FLAM_C_dup5930 | Multi | No | — | — | SINE | Alu | chr5: 31553566-31553710 | − | 63 | 145 | C5orf22 | Proximal | 0 | Not documented | 11 |
| MER57B2_dup274 | Multi | No | — | — | LTR | ERV1 | chr5: 32602884-32602920 | − | 49 | 37 | SUB1 | Proximal | 0 | Not documented | 12 |
| AluSx3_dup8129 | Multi/ | No | — | — | SINE | AIL | chr5: 36193299-36193600 | + | 45 | 302 | NADK2 | Proximal | 0 | Not documented | 13 |
| | Uniquely | | | | | | | | | | | | | | |
| L2a_dup54171 | Multi/ | No | — | — | LINE | L2 | chr5: 36193935-36194053 | + | 130 | 119 | NADK2 | Proximal | 0 | Not documented | 14 |
| | Uniquely | | | | | | | | | | | | | | |
| L2c_dup45422 | Uniquely | No | — | — | LINE | L2 | chr5: 36223603-36224030 | − | 160 | 428 | NADK2 | Proximal | 0 | Not documented | 15 |
| MIR_dup54256 | Multi | No | — | — | SINE | MIR | chr5: 36610041-36610288 | − | 110 | 248 | SLC1A3 | Proximal | 0 | Not documented | 16 |
| L1MC4a_dup7996 | Multi/ | No | — | — | LINE | Other L1 | chr5: 36617712-36618082 | − | 120 | 371 | SLC1A3 | Proximal | 0 | Not documented | 17 |
| | Uniquely | | | | | | | | | | | | | | |
| MIR3_dup27720 | Multi/ | No | — | — | SINE | MIR | chr5: 36686929-36687050 | + | 100 | 122 | SLC1A3 | Proximal | 0 | Not documented | 18 |
| | Uniquely | | | | | | | | | | | | | | |
| L2c_dup45525 | Multi | No | — | — | LINE | L2 | chr5: 38504441-38504848 | − | 170 | 408 | LIFR | Proximal | 0 | Not documented | 19 |
| L1MD2_dup3025 | Uniquely | No | — | — | LINE | Other L1 | chr5: 38505177-38505503 | + | 100 | 327 | LIFR | Proximal | 0 | Not documented | 20 |
| L1PA16_dup4433 | Uniquely | No | — | — | LINE | L1PA\|B\|x | chr5: 38907242-38910830 | + | 91 | 3589 | OSMR | Proximal | 0 | Not documented | 21 |
| L1PA16_dup4434 | Uniquely | No | — | — | LINE | L1PA\|B\|x | chr5: 38911098-38913369 | + | 65 | 2272 | OSMR | Proximal | 0 | EVE annotated | 22 |
| AluSx_dup37018 | Multi | No | — | — | SINE | Alu | chr5: 38934660-38934906 | − | 77 | 247 | OSMR | Proximal | 0 | Not documented | 23 |
| AluSq2_dup14763 | Multi/ | No | — | — | SINE | Alu | chr5: 38934930-38935236 | − | 52 | 307 | OSMR | Proximal | 0 | Not documented | 24 |
| | Uniquely | | | | | | | | | | | | | | |
| L2c_dup45681 | Multi | No | — | — | LINE | L2 | chr5: 41919736-41920067 | + | 170 | 332 | C5orf51 | Proximal | 0 | Not documented | 3625 |
| AluY_dup33658 | Multi/ | No | — | — | SINE | Alu | chr5: 43018676-43018985 | + | 13 | 310 | ANXA2R | Distal | 20351 | Not documented | 26 |
| | Uniquely | | | | | | | | | | | | | | |
| L1MB1_dup1777 | Uniquely | No | — | — | LINE | Other L1 | chr5: 43136888-43138346 | + | 72 | 1459 | ZNF131 | Proximal | 0 | Not documented | 27 |
| LTR49-int_dup638 | Multi/ | No | — | — | LTR | ERV1 | chr5: 43571615-43574124 | − | 65 | 2510 | PAIP1 | Distal | 13756 | Not documented | 28 |
| | Uniquely | | | | | | | | | | | | | | |
| AluSz_dup26065 | Multi/ | No | — | — | SINE | Alu | chr5: 43575663-43575975 | − | 62 | 313 | PAIP1 | Distal | 17804 | Not documented | 29 |
| | Uniquely | | | | | | | | | | | | | | |
| MLT1A_dup2788 | Uniquely | No | — | — | LTR | ERVL- | chr5: 44745242-44745541 | | 99 | 300 | MRPS30 | Distal | 63487 | Not documented | 30 |
| | | | | | | MaLR | | | | | | | | | |
| MIR_dup55021 | Uniquely | No | — | — | SINE | MIR | chr5: 54788514-54788717 | − | 130 | 204 | PPAP2A | Proximal | 0 | Not documented | 31 |
| AluJo_dup18876 | Uniquely | No | — | — | SINE | Alu | chr5: 54788859-54789169 | + | 85 | 311 | PPAP2A | Proximal | 0 | Not documented | 32 |
| MIRc_dup32714 | Multi | No | — | — | SINE | MIR | chr5: 55278382-55278642 | − | 160 | 261 | IL6ST | Proximal | 0 | Not documented | 33 |
| AluJb_dup39479 | Uniquely | No | — | — | SINE | Alu | chr5: 60461154-60461456 | − | 76 | 303 | SMIM15 | Distal | 2854 | Not documented | 34 |
| L1ME1_dup9857 | Multi/ | No | — | — | LINE | Other L1 | chr5: 63512676-63513493 | − | 100 | 818 | RNF180 | Proximal | 0 | Not documented | 35 |
| | Uniquely | | | | | | | | | | | | | | |
| L1ME3E_dup1609 | Uniquely | No | — | — | LINE | Other L1 | chr5: 63630344-63631839 | − | 120 | 1496 | RNF180 | Proximal | 0 | Not documented | 36 |
| L2c_dup46651 | Uniquely | No | — | — | LINE | L2 | chr5: 65330871-65331389 | + | 160 | 519 | ERBB2IP | Proximal | 0 | Not documented | 37 |
| L3_dup13950 | Multi | No | — | — | LINE | CR1 | chr5: 6606993-6607042 | + | 110 | 50 | NSUN2 | Proximal | 0 | Not documented | 38 |
| AluSx3_dup8385 | Multi/ | No | — | — | SINE | Alu | chr5: 68505820-68506125 | − | 55 | 306 | CENPH | Proximal | 0 | Not documented | 39 |
| | Uniquely | | | | | | | | | | | | | | |
| AluSx_dup37846 | Multi | No | — | — | SINE | Alu | chr5: 68629555-68629698 | + | 41 | 144 | CCDC125 | Proximal | 920 | Not documented | 40 |
| AluSx3_dup8403 | Multi | No | — | — | SINE | Alu | chr5: 69218246-69218513 | + | 54 | 268 | SERF1B | Distal | 102562 | Not documented | 41 |
| MIRb_dup70531 | Multi | No | — | — | SINE | MIR | chr5: 69274182-69274366 | − | 150 | 185 | SERF1B | Distal | 46709 | Not documented | 42 |
| L1MC4_dup9017 | Multi | No | — | — | LINE | Other L1 | chr5: 69372802-69373402 | + | 130 | 601 | SMN2 | Proximal | 0 | Not documented | 43 |
| MIRb_dup70536 | Multi | No | — | — | SINE | MIR | chr5: 69435472-69435656 | + | 150 | 185 | SMN2 | Distal | 61124 | Not documented | 44 |
| AluSx3_dup8408 | Multi | No | — | — | SINE | Alu | chr5: 69491263-69491530 | − | 54 | 268 | SMN2 | Distal | 116915 | Not documented | 45 |
| MIRb_dup70549 | Multi | No | — | — | SINE | MIR | chr5: 70149254-70149438 | − | 150 | 185 | SERF1A | Distal | 47055 | Not documented | 46 |
| L1MC4_dup9022 | Multi | No | — | — | LINE | Other L1 | chr5: 70248222-70248822 | + | 130 | 601 | SMN1 | Proximal | 0 | Not documented | 47 |
| L1MEc_dup6101 | Multi | No | — | — | LINE | Other L1 | chr5: 70339588-70340500 | − | 100 | 913 | GTF2H2 | Proximal | 0 | Not documented | 48 |
| AluSx3_dup8426 | Multi | No | — | — | SINE | Alu | chr5: 70491653-70491920 | − | 56 | 268 | GTF2H2 | Distal | 128138 | Not documented | 49 |
| MIR_dup55882 | Multi | No | — | — | SINE | MIR | chr5: 70952883-70952959 | + | 140 | 77 | MCCC2 | Proximal | 0 | Not documented | 3650 |
| L1MB3_dup4936 | Multi | No | — | — | LINE | Other L1 | chr5: 70953108-70953247 | − | 82 | 140 | MCCC2 | Proximal | 0 | Not documented | 51 |
| L2c_dup46879 | Multi | No | — | — | LINE | L2 | chr5: 70953785-70954419 | + | 150 | 635 | MCCC2 | Proximal | 0 | Not documented | 52 |
| ERV3-16A3_I- | Multi | No | — | — | LTR | ERVL | chr5: 71112573-71113395 | − | 150 | 823 | CARTPT | Distal | 95699 | Not documented | 53 |
| int_dup2217 | | | | | | | | | | | | | | | |
| THE1C-int_dup616 | Multi | No | — | — | LTR | ERVL- | chr5: 71145493-71146744 | − | 54 | 1252 | CARTPT | Distal | 128619 | Not documented | 54 |
| | | | | | | MaLR | | | | | | | | | |
| L1M4_dup4736 | Multi | No | — | — | LINE | Other L1 | chr5: 71237812-71238060 | + | 98 | 249 | MAP1B | Distal | 165002 | Not documented | 55 |
| AluJb_dup40070 | Multi/ | No | — | — | SINE | Alu | chr5: 71415349-71415633 | − | 72 | 285 | MAP1B | Proximal | 0 | Not documented | 56 |
| | Uniquely | | | | | | | | | | | | | | |
| Tigger14a_dup415 | Multi/ | No | — | — | DNA | DNA | chr5: 71423969-71424089 | − | 120 | 121 | MAP1B | Proximal | 0 | Not documented | 57 |
| | Uniquely | | | | | | | | | | | | | | |
| MIR3_dup28618 | Multi/ | No | — | — | SINE | MIR | chr5: 71426479-71426632 | + | 140 | 154 | MAP1B | Proximal | 0 | Not documented | 58 |
| | Uniquely | | | | | | | | | | | | | | |
| L2c_dup46896 | Multi | No | — | — | LINE | L2 | chr5: 71426813-71427020 | − | 110 | 208 | MAP1B | Proximal | 0 | Not documented | 59 |
| L1M6_dup2191 | Multi | No | — | — | LINE | Other L1 | chr5: 71428066-71429160 | − | 130 | 1095 | MAP1B | Proximal | 0 | Not documented | 60 |
| AluJb_dup40073 | Uniquely | No | — | — | SINE | Alu | chr5: 71435794-71436078 | − | 53 | 285 | MAP1B | Proximal | 0 | Not documented | 61 |
| MER112_dup1510 | Uniquely | No | — | — | DNA | DNA | chr5: 71436138-71436309 | − | 110 | 172 | MAP1B | Proximal | 0 | Not documented | 62 |
| AluYf4_dup422 | Multi/ | No | — | — | SINE | Alu | chr5: 71453834-71454134 | − | 39 | 301 | MAP1B | Proximal | 0 | Not documented | 63 |
| | Uniquely | | | | | | | | | | | | | | |
| MIRb_dup70610 | Multi/ | No | — | — | SINE | MIR | chr5: 71454785-71454975 | − | 120 | 191 | MAP1B | Proximal | 0 | Not documented | 64 |
| | Uniquely | | | | | | | | | | | | | | |
| MIRb_dup70611 | Uniquely | No | — | — | SINE | MIR | chr5: 71456994-71457208 | + | 160 | 215 | MAP1B | Proximal | 0 | Not documented | 65 |
| AluSx1_dup29136 | Uniquely | No | — | — | SINE | Alu | chr5: 71457502-71457811 | + | 42 | 310 | MAP1B | Proximal | 0 | Not documented | 66 |
| MIR_dup55930 | Multi | No | — | — | SINE | MIR | chr5: 71501179-71501276 | + | 130 | 98 | MAP1B | Proximal | 0 | Not documented | 67 |
| AluSz_dup26693 | Multi | No | — | — | SINE | Alu | chr5: 71516066-71516375 | + | 53 | 310 | MRPS27 | Proximal | 0 | Not documented | 68 |
| MLT1G3_dup920 | Multi | No | — | — | LTR | ERVL- | chr5: 71657014-71657451 | − | 100 | 438 | PTCD2 | Proximal | 963 | Not documented | 69 |
| | | | | | | MaLR | | | | | | | | | |
| L2b_dup30711 | Multi | No | — | — | LINE | L2 | chr5: 71657486-71657857 | + | 130 | 372 | PTCD2 | Proximal | 1435 | Not documented | 70 |
| MIR3_dup28664 | Multi | No | — | — | SINE | MIR | chr5: 72570468-72570567 | − | 120 | 100 | TMEM174 | Distal | 99499 | Not documented | 71 |
| AluSz_dup26805 | Multi | No | — | — | SINE | Alu | chr5: 74894785-74895073 | + | 60 | 289 | POLK | Proximal | 0 | Not documented | 72 |
| LTR5_Hs_dup197 | Multi | No | — | — | LTR | ERVK | chr5: 74901658-74902617 | − | 3.6 | 960 | ANKDD1B | Distal | 4668 | Not documented | 73 |
| L4_dup5552 | Multi | No | — | — | LINE | RTE | chr5: 75537276-75537341 | − | 140 | 66 | SV2C | Proximal | 0 | Not documented | 74 |
| L2c_dup47097 | Multi | No | — | — | LINE | L2 | chr5: 76030431-76030511 | − | 100 | 81 | F2R | Proximal | 0 | Not documented | 3675 |
| AluY_dup34658 | Multi/ | No | — | — | SINE | Alu | chr5: 76031017-76031328 | + | 28 | 312 | F2R | Proximal | 0 | Not documented | 76 |
| | Uniquely | | | | | | | | | | | | | | |
| MER4D1_dup395 | Uniquely | No | — | — | LTR | ERV1 | chr5: 77069787-77070640 | + | 47 | 854 | TBCA | Proximal | 0 | Not documented | 77 |
| L1PA7_dup4291 | Uniquely | No | — | — | LINE | L1PA\|B\|x | chr5: 79062222-79066557 | − | 43 | 4336 | CMYA5 | Proximal | 0 | EVE annotated | 78 |
| L2_dup18690 | Multi | No | — | — | LINE | L2 | chr5: 80748285-80748669 | − | 180 | 385 | SSBP2 | Proximal | 0 | Not documented | 79 |
| L1PREC2_dup2472 | Multi | No | — | — | LINE | L1PA\|B\|x | chr5: 82350368-82350663 | + | 29 | 296 | TMEM167A | Proximal | 0 | Not documented | 80 |
| L1PB2_dup880 | Uniquely | No | — | — | LINE | L1PA\|B\|x | chr5: 87333291-87335515 | − | 52 | 2225 | TMEM161B | Distal | 149936 | EVE annotated | 81 |
| MSTB_dup2627 | Multi | No | — | — | LTR | ERVL- | chr5: 87577602-87578004 | + | 63 | 403 | TMEM161B | Distal | 12310 | Not documented | 82 |
| | | | | | | MaLR | | | | | | | | | |
| Tigger7_dup1009 | Multi/ | No | — | — | DNA | DNA | chr5: 87578401-87578901 | − | 79 | 501 | TMEM161B | Distal | 13109 | Not documented | 83 |
| | Uniquely | | | | | | | | | | | | | | |
| L3_dup15000 | Multi | No | — | — | LINE | CR1 | chr5: 87581457-87581633 | + | 120 | 177 | TMEM161B | Distal | 16165 | Not documented | 84 |
| MLT1D_dup6334 | Multi | No | — | — | LTR | ERVL- | chr5: 87583077-87583516 | + | 110 | 440 | TMEM161B | Distal | 17785 | Not documented | 85 |
| | | | | | | MaLR | | | | | | | | | |
| L1MA3_dup3098 | Multi/ | No | — | — | LINE | Other L1 | chr5: 87632806-87635704 | + | 52 | 2899 | TMEM161B | Distal | 67514 | Not documented | 86 |
| | Uniquely | | | | | | | | | | | | | | |
| MIRc_dup33620 | Multi | No | — | — | SINE | MIR | chr5: 87705852-87705952 | + | 130 | 101 | TMEM161B | Distal | 140560 | Not documented | 87 |
| L1PA6_dup2038 | Uniquely | No | — | — | LINE | L1PA\|B\|x | chr5: 87913271-87919418 | − | 34 | 6148 | MEF2C | Distal | 94558 | EVE annotated | 88 |
| CER_dup12 | Multi | No | — | — | Other | Other | chr5: 92954798-92954836 | + | 47 | 39 | FAM172A | Proximal | 0 | Not documented | 89 |
| | | | | | | Repeats | | | | | | | | | |
| MER45A_dup1104 | Multi/ | No | — | — | DNA | DNA | chr5: 95129501-95129660 | + | 96 | 160 | GLRX | Proximal | 0 | Not documented | 90 |
| | Uniquely | | | | | | | | | | | | | | |
| AluSz_dup27292 | Multi/ | No | — | — | SINE | Alu | chr5: 95130525-95130822 | + | 47 | 298 | GLRX | Proximal | 0 | Not documented | 91 |
| | Uniquely | | | | | | | | | | | | | | |
| Charlie4z_dup1935 | Multi/ | No | — | — | DNA | DNA | chr5: 96427884-96428017 | + | 110 | 134 | LIX1 | Proximal | 0 | Not documented | 92 |
| | Uniquely | | | | | | | | | | | | | | |
| AluSx1_dup35828 | Multi | No | — | — | SINE | Alu | chr6: 107113907-107114218 | + | 48 | 312 | QRSL1 | Proximal | 0 | Not documented | 93 |
| L3_dup18093 | Multi | No | — | — | LINE | CR1 | chr6: 108194030-108194135 | + | 130 | 106 | SEC63 | Proximal | 0 | Not documented | 94 |
| MLT1F_dup1531 | Multi | No | — | — | LTR | ERVL- | chr6: 10829257-10829478 | + | 68 | 222 | MAK | Proximal | 0 | Not documented | 95 |
| | | | | | | MaLR | | | | | | | | | |
| L1MB5_dup2769 | Uniquely | No | — | — | LINE | Other L1 | chr6: 111708225-111708625 | + | 100 | 401 | REV3L | Proximal | 0 | Not documented | 96 |
| L1MA4A_dup2476 | Uniquely | No | — | — | LINE | Other L1 | chr6: 111715037-111715909 | − | 63 | 873 | REV3L | Proximal | 0 | Not documented | 97 |
| L1MA4A_dup2477 | Uniquely | No | — | — | LINE | Other L1 | chr6: 111716195-111717238 | − | 72 | 1044 | REV3L | Proximal | 0 | Not documented | 98 |
| L1M5_dup24489 | Uniquely | No | — | — | LINE | Other L1 | chr6: 111765521-111765843 | + | 98 | 323 | REV3L | Proximal | 0 | Not documented | 99 |
| L1PA16_dup5647 | Uniquely | No | — | — | LINE | L1PA\|B\|x | chr6: 111779226-111781636 | + | 51 | 2411 | REV3L | Proximal | 0 | Not documented | 3700 |
| L1MDb_dup330 | Multi | No | — | — | LINE | Other L1 | chr6: 111804112-111804305 | + | 130 | 194 | REV3L | Proximal | 0 | Not documented | 1 |
| LTR67B_dup1674 | Multi | No | — | — | LTR | ERVL | chr6: 111897594-111897912 | + | 130 | 319 | TRAF3IP2 | Proximal | 0 | Not documented | 2 |
| MIRb_dup87409 | Uniquely | No | — | — | SINE | MIR | chr6: 112133922-112134060 | + | 150 | 139 | FYN | Proximal | 0 | Not documented | 3 |
| MIR_dup69284 | Uniquely | No | — | — | SINE | MIR | chr6: 112134838-112134920 | + | 130 | 83 | FYN | Proximal | 0 | Not documented | 4 |
| MLT1J2_dup2808 | Uniquely | No | — | — | LTR | ERVL- | chr6: 112191243-112191678 | + | 120 | 436 | FYN | Proximal | 0 | Not documented | 5 |
| | | | | | | MaLR | | | | | | | | | |
| L1MA2_dup2686 | Uniquely | No | — | — | LINE | Other L1 | chr6: 118760710-118763642 | − | 49 | 2933 | CEP85L | Distal | 18294 | Not documented | 6 |
| AluJb_dup49683 | Multi | No | — | — | SINE | Alu | chr6: 122765166-122765430 | − | 74 | 265 | SERINC1 | Proximal | 0 | Not documented | 7 |
| AluSg_dup14607 | Multi | No | — | — | SINE | Alu | chr6: 126300618-126300912 | + | 37 | 295 | HINT3 | Proximal | 0 | Not documented | 8 |
| L2_dup20731 | Uniquely | No | — | — | LINE | L2 | chr6: 13097871-13099281 | + | 150 | 1411 | PHACTR1 | Proximal | 0 | Not documented | 9 |
| L2c_dup58528 | Multi | No | — | — | LINE | L2 | chr6: 133337845-133338293 | − | 150 | 449 | RPS12 | Distal | 199143 | Not documented | 10 |
| AluSp_dup17595 | Multi | No | — | — | SINE | Alu | chr6: 133359216-133359534 | − | 41 | 319 | EYA4 | Distal | 202203 | Not documented | 11 |
| MIRb_dup88852 | Multi | No | — | — | SINE | MIR | chr6: 135816873-135817085 | + | 140 | 213 | AHI1 | Proximal | 0 | Not documented | 12 |
| LTR78_dup2206 | Multi | No | — | — | LTR | ERV1 | chr6: 139017811-139017938 | − | 140 | 128 | NHSL1 | Distal | 4104 | Not documented | 13 |
| LTR78_dup2207 | Multi/ | No | — | — | LTR | ERV1 | chr6: 139018242-139018571 | − | 130 | 330 | NHSL1 | Distal | 4535 | Not documented | 14 |
| | Uniquely | | | | | | | | | | | | | | |
| MIR_dup70884 | Multi | No | — | — | SINE | MIR | chr6: 142541601-142541734 | + | 130 | 134 | VTA1 | Proximal | 0 | Not documented | 15 |
| L2a_dup71151 | Uniquely | No | — | — | LINE | L2 | chr6: 143270683-143271115 | − | 120 | 433 | HIVEP2 | Distal | 4346 | Not documented | 16 |
| L1MA7_dup3258 | Multi | No | — | — | LINE | Other L1 | chr6: 150163400-150163753 | − | 69 | 354 | LRP11 | Proximal | 0 | Not documented | 17 |
| AluSx1_dup37093 | Uniquely | No | — | — | SINE | Alu | chr6: 151163850-151164147 | − | 46 | 298 | PLEKHG1 | Proximal | 0 | Not documented | 18 |
| AluSg_dup14955 | Multi | No | — | — | SINE | Alu | chr6: 151725995-151726304 | − | 63 | 310 | RMND1 | Proximal | 0 | Not documented | 19 |
| MLT2D_dup2003 | Multi | No | — | — | LTR | ERVL | chr6: 153309705-153310018 | + | 120 | 314 | MTRF1L | Proximal | 0 | Not documented | 20 |
| LTR41B_dup405 | Multi | No | — | — | LTR | ERVL | chr6: 153310144-153310411 | + | 100 | 268 | MTRF1L | Proximal | 0 | Not documented | 21 |
| MER47C_dup322 | Multi | No | — | — | DNA | DNA | chr6: 153310591-153310674 | + | 92 | 84 | MTRF1L | Proximal | 0 | Not documented | 22 |
| Tigger5_dup1075 | Multi | No | — | — | DNA | DNA | chr6: 153310689-153310760 | − | 110 | 72 | MTRF1L | Proximal | 0 | Not documented | 23 |
| Tigger5_dup1076 | Multi | No | — | — | DNA | DNA | chr6: 153310783-153310842 | + | 76 | 60 | MTRF1L | Proximal | 0 | Not documented | 24 |
| L2c_dup59503 | Uniquely | No | — | — | LINE | L2 | chr6: 153418608-153419429 | − | 150 | 822 | RGS17 | Proximal | 0 | Not documented | 3725 |
| L1M1_dup3689 | Multi/ | No | — | — | LINE | Other L1 | chr6: 159972589-159974224 | − | 51 | 1636 | SOD2 | Distal | 115866 | Not documented | 26 |
| | Uniquely | | | | | | | | | | | | | | |
| LTR43_dup128 | Uniquely | No | — | — | LTR | ERV1 | chr6: 159995791-159996372 | + | 58 | 582 | SOD2 | Distal | 93718 | Not documented | 27 |
| L1M5_dup25571 | Multi/ | No | — | — | LINE | Other L1 | chr6: 160027483-160029255 | − | 110 | 1773 | SOD2 | Distal | 60835 | Not documented | 28 |
| | Uniquely | | | | | | | | | | | | | | |
| L1PA16_dup5849 | Multi | No | — | — | LINE | L1PA\|B\|x | chr6: 160050627-160051081 | − | 50 | 455 | SOD2 | Distal | 39009 | Not documented | 29 |
| AluSx_dup48865 | Multi | No | — | — | SINE | Alu | chr6: 160051104-160051384 | + | 57 | 281 | SOD2 | Distal | 38706 | Not documented | 30 |
| HAL1_dup11557 | Uniquely | No | — | — | LINE | Other L1 | chr6: 160052301-160053169 | − | 140 | 869 | SOD2 | Distal | 36921 | Not documented | 31 |
| AluSz_dup34074 | Multi/ | No | — | — | SINE | Alu | chr6: 160054708-160054990 | + | 45 | 283 | SOD2 | Distal | 35100 | Not documented | 32 |
| | Uniquely | | | | | | | | | | | | | | |
| AluJo_dup24524 | Multi | No | — | — | SINE | Alu | chr6: 160067582-160067897 | − | 96 | 316 | SOD2 | Distal | 22193 | Not documented | 33 |
| L2c_dup59759 | Multi | No | — | — | LINE | L2 | chr6: 160067898-160068019 | − | 120 | 122 | SOD2 | Distal | 22071 | Not documented | 34 |
| AluSx3_dup10856 | Multi | No | — | — | SINE | Alu | chr6: 160080288-160080585 | − | 43 | 298 | SOD2 | Distal | 9505 | Not documented | 35 |
| L1PBa1_dup160 | Multi/ | No | — | — | LINE | L1PA\|B\|x | chr6: 160080586-160081483 | + | 36 | 898 | SOD2 | Distal | 8607 | Not documented | 36 |
| | Uniquely | | | | | | | | | | | | | | |
| L1PBa1_dup161 | Multi | No | — | — | LINE | L1PA\|B\|x | chr6: 160082281-160083034 | + | 34 | 754 | SOD2 | Distal | 7056 | Not documented | 37 |
| LTR12 _{——} dup232 | Multi | No | — | — | LTR | ERV1 | chr6: 160083035-160083691 | + | 40 | 657 | SOD2 | Distal | 6399 | Not documented | 38 |
| L2a_dup72141 | Multi/ | No | — | — | LINE | L2 | chr6: 160085570-160086237 | − | 140 | 668 | SOD2 | Distal | 3853 | Not documented | 39 |
| | Uniquely | | | | | | | | | | | | | | |
| AluSp_dup18031 | Multi/ | No | — | — | SINE | Alu | chr6: 160086238-160086524 | + | 40 | 287 | SOD2 | Distal | 3566 | Not documented | 40 |
| | Uniquely | | | | | | | | | | | | | | |
| MLT1K_dup7542 | Uniquely | No | — | — | LTR | ERVL- | chr6: 160086745-160087114 | − | 130 | 370 | SOD2 | Distal | 2976 | Not documented | 41 |
| | | | | | | MaLR | | | | | | | | | |
| AluSp_dup18032 | Multi/ | No | — | — | SINE | Alu | chr6: 160088670-160088979 | + | 47 | 310 | SOD2 | Proximal | 1111 | Not documented | 42 |
| | Uniquely | | | | | | | | | | | | | | |
| AluSp_dup18033 | Multi/ | No | — | — | SINE | Alu | chr6: 160089338-160089646 | + | 44 | 309 | SOD2 | Proximal | 444 | Not documented | 43 |
| | Uniquely | | | | | | | | | | | | | | |
| AluSz_dup34078 | Multi/ | No | — | — | SINE | Alu | chr6: 160100828-160100958 | − | 45 | 131 | SOD2 | Proximal | 0 | Not documented | 44 |
| | Uniquely | | | | | | | | | | | | | | |
| AluSz_dup34079 | Multi/ | No | — | — | SINE | Alu | chr6: 160101686-160101990 | + | 52 | 305 | SOD2 | Proximal | 0 | Not documented | 45 |
| | Uniquely | | | | | | | | | | | | | | |
| L1M5_dup25572 | Multi | No | — | — | LINE | Other L1 | chr6: 160102001-160102101 | + | 130 | 101 | SOD2 | Proximal | 0 | Not documented | 46 |
| LTR39_dup404 | Multi/ | No | — | — | LTR | ERV1 | chr6: 160102172-160102969 | + | 73 | 798 | SOD2 | Proximal | 0 | Not documented | 47 |
| | Uniquely | | | | | | | | | | | | | | |
| AluSx_dup48867 | Multi | No | — | — | SINE | Alu | chr6: 160105368-160105668 | + | 45 | 301 | SOD2 | Proximal | 0 | Not documented | 48 |
| L2b_dup39414 | Multi | No | — | — | LINE | L2 | chr6: 163850979-163851166 | − | 160 | 188 | QKI | Proximal | 0 | Not documented | 49 |
| MER103C_dup3872 | Multi | No | — | — | DNA | DNA | chr6: 163851849-163852036 | − | 120 | 188 | QKI | Proximal | 0 | Not documented | 3750 |
| L1MD_dup3316 | Multi | No | — | — | LINE | Other L1 | chr6: 163874170-163874403 | − | 130 | 234 | QKI | Proximal | 0 | Not documented | 51 |
| L2c_dup59886 | Multi | No | — | — | LINE | L2 | chr6: 163890571-163890673 | + | 110 | 103 | QKI | Proximal | 0 | Not documented | 52 |
| Tigger2_dup1197 | Multi | No | — | — | DNA | DNA | chr6: 163893055-163893483 | − | 68 | 429 | QKI | Proximal | 0 | Not documented | 53 |
| Tigger1_dup5301 | Multi | No | — | — | DNA | DNA | chr6: 163904066-163905231 | − | 50 | 1166 | QKI | Proximal | 0 | Not documented | 54 |
| AluSz6_dup16629 | Multi | No | — | — | SINE | Alu | chr6: 163905232-163905532 | − | 60 | 301 | QKI | Proximal | 0 | Not documented | 55 |
| Tigger1_dup5302 | Multi | No | — | — | DNA | DNA | chr6: 163905533-163905718 | − | 50 | 186 | QKI | Proximal | 0 | Not documented | 56 |
| Tigger1_dup5303 | Multi | No | — | — | DNA | DNA | chr6: 163905714-163906028 | − | 84 | 315 | QKI | Proximal | 0 | Not documented | 57 |
| MER21C_dup2086 | Multi | No | — | — | LTR | ERVL | chr6: 163906029-163906118 | − | 88 | 90 | QKI | Proximal | 0 | Not documented | 58 |
| MER5C_dup582 | Multi | No | — | — | DNA | DNA | chr6: 163912164-163912379 | − | 110 | 216 | QKI | Proximal | 0 | Not documented | 59 |
| MIR_dup71945 | Multi | No | — | — | SINE | MIR | chr6: 163917327-163917518 | − | 120 | 192 | QKI | Proximal | 0 | Not documented | 60 |
| L2a_dup72322 | Multi | No | — | — | LINE | L2 | chr6: 163917529-163918110 | + | 130 | 582 | QKI | Proximal | 0 | Not documented | 61 |
| MER21B_dup1055 | Multi | No | — | — | LTR | ERVL | chr6: 163972152-163972441 | − | 84 | 290 | QKI | Proximal | 0 | Not documented | 62 |
| L1MC1_dup5765 | Uniquely | No | — | — | LINE | Other L1 | chr6: 165684911-165686061 | + | 79 | 1151 | C6orf118 | Distal | 7093 | Not documented | 63 |
| Tigger1_dup5304 | Multi | No | — | — | DNA | DNA | chr6: 165788184-165790121 | + | 65 | 1938 | PDE10A | Proximal | 0 | Not documented | 64 |
| L1ME3E_dup1809 | Uniquely | No | — | — | LINE | Other L1 | chr6: 16623135-16623566 | − | 130 | 432 | ATXN1 | Proximal | 0 | Not documented | 65 |
| MER136_dup13 | Multi | No | — | — | DNA | DNA | chr6: 169616834-169616966 | + | 130 | 133 | THBS2 | Proximal | 0 | Not documented | 66 |
| L1ME4a_dup17799 | Multi | No | — | — | LINE | Other L1 | chr6: 170714061-170714179 | − | 140 | 119 | FAM120B | Proximal | 0 | Not documented | 67 |
| L2b_dup39552 | Multi | No | — | — | LINE | L2 | chr6: 170891224-170891414 | + | 130 | 191 | PDCD2 | Proximal | 0 | Not documented | 68 |
| AluSc8_dup7019 | Uniquely | No | — | — | SINE | Alu | chr6: 17477093-17477404 | − | 32 | 312 | CAP2 | Proximal | 0 | Not documented | 69 |
| HAL1_dup10075 | Multi | No | — | — | LINE | Other L1 | chr6: 17619096-17619716 | + | 120 | 621 | NUP153 | Proximal | 0 | Not documented | 70 |
| L1MA4_dup3639 | Multi | No | — | — | LINE | Other L1 | chr6: 17619866-17620046 | + | 64 | 181 | NUP153 | Proximal | 0 | Not documented | 71 |
| AluSx_dup43269 | Multi | No | — | — | SINE | Alu | chr6: 17620047-17620352 | + | 48 | 306 | NUP153 | Proximal | 0 | Not documented | 72 |
| Tigger1_dup4661 | Uniquely | No | — | — | DNA | DNA | chr6: 17684477-17685365 | − | 64 | 889 | NUP153 | Proximal | 0 | Not documented | 73 |
| AluY_dup39207 | Multi | No | — | — | SINE | Alu | chr6: 17685501-17685796 | + | 35 | 296 | NUP153 | Proximal | 0 | Not documented | 74 |
| L2b_dup35512 | Uniquely | No | — | — | LINE | L2 | chr6: 22131875-22133351 | + | 150 | 1477 | PRL | Distal | 154130 | Not documented | 3775 |
| MIR_dup64505 | Uniquely | No | — | — | SINE | MIR | chr6: 24423301-24423509 | + | 130 | 209 | MRS2 | Proximal | 0 | Not documented | 76 |
| AluSx1_dup33631 | Multi | No | — | — | SINE | Alu | chr6: 26127949-26128240 | + | 48 | 292 | HIST1H2AC | Proximal | 0 | Not documented | 77 |
| L1MC4a_dup9745 | Multi | No | — | — | LINE | Other L1 | chr6: 26138670-26138926 | − | 110 | 257 | HIST1H2AC | Proximal | 0 | Not documented | 78 |
| MIRb_dup82378 | Multi | No | — | — | SINE | MIR | chr6: 26139020-26139141 | + | 150 | 122 | HIST1H2AC | Proximal | 0 | Not documented | 79 |
| L2a_dup64505 | Multi/ | No | — | — | LINE | L2 | chr6: 26280939-26281729 | − | 130 | 791 | HIST1H4H | Proximal | 0 | Not documented | 80 |
| | Uniquely | | | | | | | | | | | | | | |
| L1ME3E_dup1838 | Multi | No | — | — | LINE | Other L1 | chr6: 26544430-26545362 | − | 120 | 933 | HMGN4 | Proximal | 0 | Not documented | 81 |
| L1ME3E_dup1839 | Multi/ | No | — | — | LINE | Other L1 | chr6: 26546483-26547423 | − | 120 | 941 | HMGN4 | Proximal | 2 | Not documented | 82 |
| | Uniquely | | | | | | | | | | | | | | |
| Tigger10_dup376 | Multi | No | — | — | DNA | DNA | chr6: 28245193-28245328 | + | 100 | 136 | PGBD1 | Distal | 3987 | Not documented | 83 |
| Arthur1C_dup266 | Multi | No | — | — | DNA | DNA | chr6: 29597136-29597210 | − | 79 | 75 | GABBR1 | Proximal | 0 | Not documented | 84 |
| Arthur1B_dup915 | Multi | No | — | — | DNA | DNA | chr6: 29597213-29597382 | − | 120 | 170 | GABBR1 | Proximal | 0 | Not documented | 85 |
| AluJb_dup46022 | Multi | No | — | — | SINE | Alu | chr6: 30246492-30246788 | − | 75 | 297 | TRIM39 | Distal | 47469 | Not documented | 86 |
| MSTA-int_dup1177 | Uniquely | No | — | — | LTR | ERVL- | chr6: 30251053-30252681 | − | 70 | 1629 | TRIM39 | Distal | 41576 | Not documented | 87 |
| | | | | | | MaLR | | | | | | | | | |
| AluJb_dup46040 | Multi/ | No | — | — | SINE | Alu | chr6: 30593596-30593888 | + | 62 | 293 | MRPS18B | Proximal | 0 | Not documented | 88 |
| | Uniquely | | | | | | | | | | | | | | |
| Charlie4a_dup1191 | Multi | No | — | — | DNA | DNA | chr6: 30868201-30868298 | − | 98 | 98 | DDR1 | Proximal | 269 | Not documented | 89 |
| AluSz6_dup14840 | Multi/ | No | — | — | SINE | Alu | chr6: 30868299-30868606 | + | 59 | 308 | DDR1 | Proximal | 367 | Not documented | 90 |
| | Uniquely | | | | | | | | | | | | | | |
| AluJr_dup22900 | Multi | No | — | — | SINE | Alu | chr6: 3152979-3153295 | + | 73 | 317 | BPHL | Proximal | 0 | Not documented | 91 |
| AluSx1_dup33891 | Multi | No | — | — | SINE | Alu | chr6: 31721509-31721802 | − | 43 | 294 | MSH5-SAPCD1 | Proximal | 0 | Not documented | 92 |
| AluJb_dup46181 | Multi | No | — | — | SINE | Alu | chr6: 31804050-31804312 | − | 91 | 263 | C6orf48 | Proximal | 0 | Not documented | 93 |
| L2a_dup64831 | Multi | No | — | — | LINE | L2 | chr6: 32162775-32162828 | + | 59 | 54 | GPSM3 | Proximal | 0 | Not documented | 94 |
| Charlie1b_dup1202 | Multi | No | — | — | DNA | DNA | chr6: 33268874-33269007 | + | 68 | 134 | TAPBP | Proximal | 0 | Not documented | 95 |
| AluSx_dup44245 | Multi | No | — | — | SINE | Alu | chr6: 33269069-33269372 | + | 46 | 304 | TAPBP | Proximal | 0 | Not documented | 96 |
| L2_dup21096 | Multi | No | — | — | LINE | L2 | chr6: 33269400-33269529 | − | 160 | 130 | TAPBP | Proximal | 0 | Not documented | 97 |
| AluSz6_dup14901 | Multi | No | — | — | SINE | Alu | chr6: 34253854-34254157 | − | 58 | 304 | NUDT3 | Proximal | 0 | Not documented | 98 |
| AluJr_dup23958 | Multi | No | — | — | SINE | Alu | chr6: 36569142-36569415 | − | 90 | 274 | SRSF3 | Proximal | 0 | Not documented | 99 |
| AluSx1_dup34256 | Multi | No | — | — | SINE | Alu | chr6: 36894028-36894336 | + | 55 | 309 | C6orf89 | Proximal | 0 | Not documented | 3800 |
| MIRb_dup83161 | Multi | No | — | — | SINE | MIR | chr6: 38644378-38644456 | + | 120 | 79 | GLO1 | Proximal | 0 | Not documented | 1 |
| AluSc_dup11840 | Multi/ | No | — | — | SINE | Alu | chr6: 42174959-42175257 | − | 35 | 299 | MRPS10 | Proximal | 0 | Not documented | 2 |
| | Uniquely | | | | | | | | | | | | | | |
| AluSx_dup45001 | Multi | No | — | — | SINE | Alu | chr6: 42712364-42712524 | + | 37 | 161 | TBCC | Proximal | 0 | Not documented | 3 |
| AluSc_dup11857 | Multi | No | — | — | SINE | Alu | chr6: 42854452-42854749 | + | 40 | 298 | RPL7L1 | Proximal | 0 | Not documented | 4 |
| AluSp_dup16689 | Multi | No | — | — | SINE | Alu | chr6: 42855039-42855333 | + | 37 | 295 | RPL7L1 | Proximal | 0 | Not documented | 5 |
| AluSp_dup16690 | Multi | No | — | — | SINE | Alu | chr6: 42856897-42857198 | + | 51 | 302 | RPL7L1 | Proximal | 0 | Not documented | 6 |
| MIRc_dup38832 | Multi | No | — | — | SINE | MIR | chr6: 42857408-42857543 | + | 150 | 136 | RPL7L1 | Proximal | 0 | Not documented | 7 |
| LTR50_dup1017 | Multi | No | — | — | LTR | ERVL | chr6: 42981127-42981210 | − | 110 | 84 | MEA1 | Proximal | 0 | Not documented | 8 |
| MIRb_dup83642 | Multi | No | — | — | SINE | MIR | chr6: 43487580-43487782 | + | 120 | 203 | POLR1C | Proximal | 0 | Not documented | 9 |
| AluSp_dup16808 | Uniquely | No | — | — | SINE | Alu | chr6: 49520634-49520942 | − | 53 | 309 | C6orf141 | Proximal | 0 | Not documented | 10 |
| 7SK_dup280 | Multi | No | — | — | Other | Other | chr6: 52860418-52860749 | + | 1.4 | 332 | GSTA4 | Proximal | 243 | Not documented | 11 |
| | | | | | | Repeats | | | | | | | | | |
| L1PA7_dup5090 | Uniquely | No | — | — | LINE | L1PA\|B\|x | chr6: 56430383-56431459 | − | 39 | 1077 | DST | Proximal | 0 | Not documented | 12 |
| MIR3_dup34637 | Multi | No | — | — | SINE | MIR | chr6: 56485808-56485914 | − | 150 | 107 | DST | Proximal | 0 | Not documented | 13 |
| AluSg7_dup2811 | Multi | No | — | — | SINE | Alu | chr6: 56487087-56487394 | + | 30 | 308 | DST | Proximal | 0 | Not documented | 14 |
| MIRb_dup84694 | Uniquely | No | — | — | SINE | MIR | chr6: 56667819-56667971 | − | 130 | 153 | DST | Proximal | 0 | Not documented | 15 |
| MLT1D_dup7286 | Uniquely | No | — | — | LTR | ERVL- | chr6: 56682576-56683032 | + | 84 | 457 | DST | Proximal | 0 | Not documented | 16 |
| | | | | | | MaLR | | | | | | | | | |
| Charlie19a_dup588 | Multi/ | No | — | — | DNA | DNA | chr6: 56683285-56683497 | − | 120 | 213 | DST | Proximal | 0 | Not documented | 17 |
| | Uniquely | | | | | | | | | | | | | | |
| MIRb_dup84697 | Multi/ | No | — | — | SINE | MIR | chr6: 56683709-56683782 | + | 130 | 74 | DST | Proximal | 0 | Not documented | 18 |
| | Uniquely | | | | | | | | | | | | | | |
| L1MEc_dup7074 | Uniquely | No | — | — | LINE | Other L1 | chr6: 56686897-56687370 | + | 110 | 474 | DST | Proximal | 0 | Not documented | 19 |
| AluY_dup40962 | Multi | No | — | — | SINE | Alu | chr6: 56701367-56701678 | + | 16 | 312 | DST | Proximal | 0 | Not documented | 20 |
| LTR12C_dup1191 | Multi | No | — | — | LTR | ERV1 | chr6: 70183132-70183433 | + | 59 | 302 | BAI3 | Distal | 83730 | Not documented | 21 |
| L1MDb_dup326 | Multi | No | — | — | LINE | Other L1 | chr6: 79787177-79787384 | + | 140 | 208 | PHIP | Proximal | 0 | Not documented | 22 |
| MIRc_dup39987 | Multi/ | No | — | — | SINE | MIR | chr6: 86204046-86204271 | − | 140 | 226 | NT5E | Proximal | 0 | Not documented | 23 |
| | Uniquely | | | | | | | | | | | | | | |
| L1PA7_dup5358 | Uniquely | No | — | — | LINE | L1PA\|B\|x | chr6: 89536814-89543297 | + | 36 | 6484 | RNGTT | Proximal | 0 | EVE annotated | 24 |
| L1MB8_dup5678 | Multi | No | — | — | LINE | Other L1 | chr6: 90345849-90346188 | − | 61 | 340 | LYRM2 | Proximal | 0 | Not documented | 3825 |
| L1MB8_dup5679 | Multi | No | — | — | LINE | Other L1 | chr6: 90346201-90346399 | − | 92 | 199 | LYRM2 | Proximal | 0 | Not documented | 26 |
| AluSz_dup32225 | Multi | No | — | — | SINE | Alu | chr6: 90346400-90346699 | − | 56 | 300 | LYRM2 | Proximal | 0 | Not documented | 27 |
| L1MB8_dup5680 | Multi | No | — | — | LINE | Other L1 | chr6: 90346700-90346842 | − | 110 | 143 | LYRM2 | Proximal | 0 | Not documented | 28 |
| L1MA9_dup6261 | Uniquely | No | — | — | LINE | Other L1 | chr6: 90967794-90970352 | − | 83 | 2559 | BACH2 | Proximal | 0 | Not documented | 29 |
| L1PB1_dup5378 | Uniquely | No | — | — | LINE | L1PA\|B\|x | chr6: 96933381-96937016 | − | 48 | 3636 | UFL1 | Distal | 32456 | EVE annotated | 30 |
| L1PA16_dup5560 | Uniquely | No | — | — | LINE | L1PA\|B\|x | chr6: 98750169-98752262 | − | 50 | 2094 | POU3F2 | Distal | 530319 | Not documented | 31 |
| L1MA8_dup3967 | Multi/ | No | — | — | LINE | Other L1 | chr6: 99298129-99299827 | + | 67 | 1699 | POU3F2 | Distal | 11470 | Not documented | 32 |
| | Uniquely | | | | | | | | | | | | | | |
| AluJr_dup25007 | Multi | No | — | — | SINE | Alu | chr6: 99859488-99859727 | − | 65 | 240 | PNISR | Proximal | 0 | Not documented | 33 |
| AluSz6_dup18386 | Multi | No | — | — | SINE | Alu | chr7: 100145958-100146235 | − | 57 | 278 | AGFG2 | Proximal | 0 | Not documented | 34 |
| AluJr_dup30493 | Multi | No | — | — | SINE | Alu | chr7: 100733625-100733916 | + | 64 | 292 | TRIM56 | Proximal | 0 | Not documented | 35 |
| MIR3_dup39611 | Multi | No | — | — | SINE | MIR | chr7: 100780892-100781040 | − | 160 | 149 | SERPINE1 | Proximal | 0 | Not documented | 36 |
| L2a_dup77382 | Multi | No | — | — | LINE | L2 | chr7: 100957915-100958041 | + | 140 | 127 | RABL5 | Proximal | 0 | Not documented | 37 |
| MSTA_dup8743 | Multi | No | — | — | LTR | ERVL- | chr7: 101982409-101982792 | + | 58 | 384 | SH2B2 | Distal | 20232 | Not documented | 38 |
| | | | | | | MaLR | | | | | | | | | |
| AluSq2_dup21950 | Multi | No | — | — | SINE | Alu | chr7: 101983142-101983442 | + | 65 | 301 | PRKRIP1 | Distal | 20878 | Not documented | 39 |
| AluSg7_dup3345 | Multi | No | — | — | SINE | Alu | chr7: 101983445-101983745 | + | 59 | 301 | PRKRIP1 | Distal | 20575 | Not documented | 40 |
| HERV4_I-int_{—} | Multi | No | — | — | LTR | ERV1 | chr7: 101983977-101984243 | − | 150 | 267 | PRKRIP1 | Distal | 20077 | Not documented | 41 |
| dup150 | | | | | | | | | | | | | | | |
| AluSz6_dup18469 | Multi | No | — | — | SINE | Alu | chr7: 101993041-101993380 | − | 70 | 340 | PRKRIP1 | Distal | 10940 | Not documented | 42 |
| AluSz_dup38343 | Multi | No | — | — | SINE | Alu | chr7: 101994464-101994764 | − | 75 | 301 | PRKRIP1 | Distal | 9556 | Not documented | 43 |
| AluJo_dup28163 | Multi | No | — | — | SINE | Alu | chr7: 101994769-101994924 | − | 76 | 156 | PRKRIP1 | Distal | 9396 | Not documented | 44 |
| FLAM_A_dup6889 | Multi/ | No | — | — | SINE | Alu | chr7: 102066571-102066693 | + | 85 | 123 | PRKRIP1 | Proximal | 0 | Not documented | 45 |
| | Uniquely | | | | | | | | | | | | | | |
| AluSx1_dup42691 | Multi | No | — | — | SINE | Alu | chr7: 102088873-102089179 | + | 28 | 307 | ORAI2 | Proximal | 0 | Not documented | 46 |
| AluSq2_dup21959 | Multi | No | — | — | SINE | Alu | chr7: 102089180-102089343 | + | 42 | 164 | ORAI2 | Proximal | 0 | Not documented | 47 |
| AluSx1_dup42692 | Multi | No | — | — | SINE | Alu | chr7: 102089870-102090176 | + | 56 | 307 | ORAI2 | Proximal | 0 | Not documented | 48 |
| AluSx_dup55338 | Multi | No | — | — | SINE | Alu | chr7: 102091366-102091666 | + | 39 | 301 | ORAI2 | Proximal | 0 | Not documented | 49 |
| AluSz_dup38352 | Multi/ | No | — | — | SINE | Alu | chr7: 102097281-102097575 | − | 51 | 295 | ALKBH4 | Proximal | 0 | Not documented | 3850 |
| | Uniquely | | | | | | | | | | | | | | |
| AluSc_dup14435 | Multi | No | — | — | SINE | Alu | chr7: 102178460-102178755 | + | 43 | 296 | POLR2J3 | Proximal | 0 | Not documented | 51 |
| AluJo_dup28187 | Multi | No | — | — | SINE | Alu | chr7: 102195248-102195407 | + | 82 | 160 | SPDYE2 | Proximal | 0 | Not documented | 52 |
| AluSz_dup38365 | Multi | No | — | — | SINE | Alu | chr7: 102195408-102195709 | + | 71 | 302 | SPDYE2 | Proximal | 0 | Not documented | 53 |
| AluSz6_dup18479 | Multi | No | — | — | SINE | Alu | chr7: 102196798-102197137 | + | 70 | 340 | SPDYE2 | Proximal | 0 | Not documented | 54 |
| AluY_dup49034 | Multi | No | — | — | SINE | Alu | chr7: 102205128-102205437 | − | 32 | 310 | POLR2J3 | Proximal | 0 | Not documented | 55 |
| MER21C_dup2389 | Multi | No | — | — | LTR | ERVL | chr7: 102205444-102205658 | − | 120 | 215 | POLR2J3 | Proximal | 0 | Not documented | 56 |
| AluSx_dup55370 | Multi | No | — | — | SINE | Alu | chr7: 102205665-102205875 | − | 48 | 211 | POLR2J3 | Proximal | 0 | Not documented | 57 |
| HERV4_I-int_{—} | Multi | No | — | — | LTR | ERV1 | chr7: 102206001-102206303 | + | 140 | 303 | POLR2J3 | Proximal | 0 | Not documented | 58 |
| dup151 | | | | | | | | | | | | | | | |
| AluSg7_dup3348 | Multi | No | — | — | SINE | Alu | chr7: 102206439-102206716 | − | 47 | 278 | POLR2J3 | Proximal | 0 | Not documented | 59 |
| AluSq2_dup21967 | Multi | No | — | — | SINE | Alu | chr7: 102206720-102207018 | − | 67 | 299 | POLR2J3 | Proximal | 0 | Not documented | 60 |
| HERV4_I-int_{—} | Multi | No | — | — | LTR | ERV1 | chr7: 102207072-102207188 | + | 160 | 117 | POLR2J3 | Proximal | 0 | Not documented | 61 |
| dup152 | | | | | | | | | | | | | | | |
| MER84-int_dup71 | Multi | No | — | — | LTR | ERV1 | chr7: 102207217-102207343 | + | 160 | 127 | POLR2J3 | Proximal | 0 | Not documented | 62 |
| MSTA_dup8746 | Multi | No | — | — | LTR | ERVL- | chr7: 102207365-102207748 | − | 59 | 384 | POLR2J3 | Proximal | 0 | Not documented | 63 |
| | | | | | | MaLR | | | | | | | | | |
| AluSg_dup16738 | Multi | No | — | — | SINE | Alu | chr7: 102207794-102208027 | − | 47 | 234 | POLR2J3 | Proximal | 0 | Not documented | 64 |
| AluSc_dup14436 | Multi | No | — | — | SINE | Alu | chr7: 102277565-102277862 | + | 41 | 298 | UPK3BL | Proximal | 0 | Not documented | 65 |
| AluJo_dup28201 | Multi | No | — | — | SINE | Alu | chr7: 102294340-102294495 | + | 76 | 156 | SPDYE2B | Proximal | 0 | Not documented | 66 |
| AluSz_dup38377 | Multi | No | — | — | SINE | Alu | chr7: 102294500-102294801 | + | 75 | 302 | SPDYE2B | Proximal | 0 | Not documented | 67 |
| AluSx1_dup42735 | Multi | No | — | — | SINE | Alu | chr7: 102294862-102295181 | + | 54 | 320 | SPDYE2B | Proximal | 0 | Not documented | 68 |
| AluSz6_dup18483 | Multi | No | — | — | SINE | Alu | chr7: 102295882-102296221 | + | 70 | 340 | SPDYE2B | Proximal | 0 | Not documented | 69 |
| AluY_dup49038 | Multi | No | — | — | SINE | Alu | chr7: 102304210-102304520 | − | 32 | 311 | POLR2J2 | Proximal | 0 | Not documented | 70 |
| MER21C_dup2390 | Multi | No | — | — | LTR | ERVL | chr7: 102304527-102304741 | − | 120 | 215 | POLR2J2 | Proximal | 0 | Not documented | 71 |
| AluSx_dup55395 | Multi | No | — | — | SINE | Alu | chr7: 102304748-102304959 | − | 45 | 212 | POLR2J2 | Proximal | 0 | Not documented | 72 |
| PABL_B-int_dup98 | Multi | No | — | — | LTR | ERV1 | chr7: 102305005-102305387 | + | 160 | 383 | POLR2J2 | Proximal | 0 | Not documented | 73 |
| AluSg7_dup3351 | Multi | No | — | — | SINE | Alu | chr7: 102305498-102305796 | − | 53 | 299 | POLR2J2 | Proximal | 0 | Not documented | 74 |
| AluSq2_dup21974 | Multi | No | — | — | SINE | Alu | chr7: 102305799-102306098 | − | 68 | 300 | POLR2J2 | Proximal | 0 | Not documented | 3875 |
| PABL_B-int_dup99 | Multi | No | — | — | LTR | ERV1 | chr7: 102306129-102306286 | + | 150 | 158 | POLR2J2 | Proximal | 0 | Not documented | 76 |
| MER84-int_dup72 | Multi | No | — | — | LTR | ERV1 | chr7: 102306285-102306423 | + | 150 | 139 | POLR2J2 | Proximal | 0 | Not documented | 77 |
| MSTA_dup8747 | Multi | No | — | — | LTR | ERVL- | chr7: 102306445-102306828 | − | 58 | 384 | POLRZJ2 | Proximal | 0 | Not documented | 78 |
| | | | | | | MaLR | | | | | | | | | |
| AluSg_dup16744 | Multi | No | — | — | SINE | Alu | chr7: 102306874-102307107 | − | 47 | 234 | POLR2J2 | Proximal | 0 | Not documented | 79 |
| AluSp_dup19891 | Multi | No | — | — | SINE | Alu | chr7: 102739637-102739933 | + | 38 | 297 | ARMC10 | Proximal | 0 | Not documented | 80 |
| L1MC4_dup12978 | Multi | No | — | — | LINE | Other L1 | chr7: 102858182-102859496 | + | 120 | 1315 | NAPEPLD | Distal | 68176 | Not documented | 81 |
| AluSz6_dup18537 | Multi | No | — | — | SINE | Alu | chr7: 104647373-104647544 | − | 58 | 172 | KMT2E | Distal | 7083 | Not documented | 82 |
| MER5A_dup15748 | Uniquely | No | — | — | DNA | DNA | chr7: 104695954-104696121 | − | 160 | 168 | KMT2E | Proximal | 0 | Not documented | 83 |
| L1MDb_dup424 | Multi | No | — | — | LINE | Other L1 | chr7: 105752752-105752947 | − | 140 | 196 | SYPL1 | Proximal | 0 | Not documented | 84 |
| L1MEg_dup8309 | Uniquely | No | — | — | LINE | Other L1 | chr7: 105880569-105881651 | − | 120 | 1083 | NAMPT | Distal | 7081 | Not documented | 85 |
| MER58A_dup5723 | Multi | No | — | — | DNA | DNA | chr7: 105890032-105890226 | − | 120 | 195 | NAMPT | Proximal | 0 | Not documented | 86 |
| MER47A_dup1316 | Multi | No | — | — | DNA | DNA | chr7: 105896993-105897288 | + | 71 | 296 | NAMPT | Proximal | 0 | Not documented | 87 |
| AluSq_dup8666 | Multi | No | — | — | SINE | Alu | chr7: 105897340-105897610 | + | 45 | 271 | NAMPT | Proximal | 0 | Not documented | 88 |
| AluJr_dup30864 | Multi | No | — | — | SINE | Alu | chr7: 105897781-105898085 | + | 73 | 305 | NAMPT | Proximal | 0 | Not documented | 89 |
| Tigger1_dup5757 | Multi | No | — | — | DNA | DNA | chr7: 105899014-105899876 | − | 59 | 863 | NAMPT | Proximal | 0 | Not documented | 90 |
| Tigger1_dup5758 | Multi | No | — | — | DNA | DNA | chr7: 105900168-105901560 | − | 59 | 1393 | NAMPT | Proximal | 0 | Not documented | 91 |
| MIRb_dup97360 | Multi | No | — | — | SINE | MIR | chr7: 105903075-105903304 | + | 150 | 230 | NAMPT | Proximal | 0 | Not documented | 92 |
| MIR3_dup39767 | Multi | No | — | — | SINE | MIR | chr7: 105903647-105903731 | + | 110 | 85 | NAMPT | Proximal | 0 | Not documented | 93 |
| Tigger4a_dup1562 | Multi | No | — | — | DNA | DNA | chr7: 105904847-105905064 | − | 53 | 218 | NAMPT | Proximal | 0 | Not documented | 94 |
| MIRc_dup45365 | Multi | No | — | — | SINE | MIR | chr7: 105906366-105906614 | − | 130 | 249 | NAMPT | Proximal | 0 | Not documented | 95 |
| L2a_dup77613 | Multi | No | — | — | LINE | L2 | chr7: 105907464-105907536 | + | 140 | 73 | NAMPT | Proximal | 0 | Not documented | 96 |
| AluYa5_dup1806 | Multi | No | — | — | SINE | Alu | chr7: 105908007-105908315 | + | 5.9 | 309 | NAMPT | Proximal | 0 | Not documented | 97 |
| L4_dup8043 | Multi | No | — | — | LINE | RTE | chr7: 105909901-105910056 | + | 140 | 156 | NAMPT | Proximal | 0 | Not documented | 98 |
| MER5C1_dup286 | Multi | No | — | — | DNA | DNA | chr7: 105910124-105910258 | − | 100 | 135 | NAMPT | Proximal | 0 | Not documented | 99 |
| L4_dup8044 | Multi | No | — | — | LINE | RTE | chr7: 105910259-105910706 | + | 140 | 448 | NAMPT | Proximal | 0 | Not documented | 3900 |
| MLT1H_dup4384 | Multi/ | No | — | — | LTR | ERVL- | chr7: 105910942-105911376 | + | 100 | 435 | NAMPT | Proximal | 0 | Not documented | 1 |
| | Uniquely | | | | | MaLR | | | | | | | | | |
| MIR3_dup39768 | Multi | No | — | — | SINE | MIR | chr7: 105912794-105912925 | − | 150 | 132 | NAMPT | Proximal | 0 | Not documented | 2 |
| MIR3_dup39769 | Multi | No | — | — | SINE | MIR | chr7: 105913943-105914087 | + | 130 | 145 | NAMPT | Proximal | 0 | Not documented | 3 |
| AluSz_dup38548 | Multi | No | — | — | SINE | Alu | chr7: 105915019-105915323 | + | 57 | 305 | NAMPT | Proximal | 0 | Not documented | 4 |
| MIR_dup77466 | Multi | No | — | — | SINE | MIR | chr7: 105915947-105916084 | + | 120 | 138 | NAMPT | Proximal | 0 | Not documented | 5 |
| AluJb_dup57320 | Multi | No | — | — | SINE | Alu | chr7: 105916199-105916496 | + | 67 | 298 | NAMPT | Proximal | 0 | Not documented | 6 |
| AluSp_dup19948 | Multi/ | No | — | — | SINE | Alu | chr7: 105917007-105917304 | + | 55 | 298 | NAMPT | Proximal | 0 | Not documented | 7 |
| | Uniquely | | | | | | | | | | | | | | |
| L2c_dup64183 | Multi | No | — | — | LINE | L2 | chr7: 105920094-105920839 | − | 130 | 746 | NAMPT | Proximal | 0 | Not documented | 8 |
| AluSc8_dup8887 | Multi | No | — | — | SINE | Alu | chr7: 105921106-105921400 | + | 32 | 295 | NAMPT | Proximal | 0 | Not documented | 9 |
| MER5B_dup10992 | Multi | No | — | — | DNA | DNA | chr7: 106843373-106843559 | − | 97 | 187 | COG5 | Proximal | 0 | Not documented | 10 |
| L1MA7_dup3647 | Uniquely | No | — | — | LINE | Other L1 | chr7: 106853084-106854605 | + | 120 | 1522 | COG5 | Proximal | 0 | Not documented | 11 |
| MER61-int_dup538 | Uniquely | No | — | — | LTR | ERV1 | chr7: 107023184-107024764 | + | 41 | 1581 | COG5 | Proximal | 0 | Not documented | 12 |
| L1MCc_dup1660 | Multi | No | — | — | LINE | Other L1 | chr7: 107102448-107102914 | + | 140 | 467 | COG5 | Proximal | 0 | Not documented | 13 |
| MIR3_dup39802 | Multi | No | — | — | SINE | MIR | chr7: 107260298-107260406 | + | 160 | 109 | BCAP29 | Proximal | 0 | Not documented | 14 |
| MIRb_dup97429 | Multi | No | — | — | SINE | MIR | chr7: 107260375-107260453 | + | 130 | 79 | BCAP29 | Proximal | 0 | Not documented | 15 |
| MIR3_dup39803 | Multi | No | — | — | SINE | MIR | chr7: 107260563-107260730 | − | 150 | 168 | BCAP29 | Proximal | 0 | Not documented | 16 |
| MIR_dup77521 | Multi | No | — | — | SINE | MIR | chr7: 107260783-107260823 | − | 89 | 41 | BCAP29 | Proximal | 0 | Not documented | 17 |
| MLT1D_dup8600 | Multi | No | — | — | LTR | ERVL- | chr7: 107261903-107262361 | + | 99 | 459 | BCAP29 | Proximal | 0 | Not documented | 18 |
| | | | | | | MaLR | | | | | | | | | |
| AluSx1_dup42973 | Multi/ | No | — | — | SINE | Alu | chr7: 107806681-107806992 | − | 35 | 312 | NRCAM | Proximal | 0 | Not documented | 19 |
| | Uniquely | | | | | | | | | | | | | | |
| L1MA9_dup7332 | Multi | No | — | — | LINE | Other L1 | chr7: 107810495-107810776 | + | 55 | 282 | NRCAM | Proximal | 0 | Not documented | 20 |
| MER4A1_dup890 | Multi/ | No | — | — | LTR | ERV1 | chr7: 107810777-107811228 | + | 45 | 452 | NRCAM | Proximal | 0 | Not documented | 21 |
| | Uniquely | | | | | | | | | | | | | | |
| AluSz_dup38616 | Multi | No | — | — | SINE | Alu | chr7: 107812720-107813040 | − | 53 | 321 | NRCAM | Proximal | 0 | Not documented | 22 |
| AluSq2_dup22098 | Multi/ | No | — | — | SINE | Alu | chr7: 107813429-107813730 | − | 47 | 302 | NRCAM | Proximal | 0 | Not documented | 23 |
| | Uniquely | | | | | | | | | | | | | | |
| MER119_dup573 | Multi/ | No | — | — | DNA | DNA | chr7: 107827018-107827554 | − | 100 | 537 | NRCAM | Proximal | 0 | Not documented | 24 |
| | Uniquely | | | | | | | | | | | | | | |
| L2a_dup77754 | Multi/ | No | — | — | LINE | L2 | chr7: 107827555-107827980 | + | 110 | 426 | NRCAM | Proximal | 0 | Not documented | 3925 |
| | Uniquely | | | | | | | | | | | | | | |
| LTR16B1_dup573 | Multi | No | — | — | LTR | ERVL | chr7: 107828054-107828113 | − | 110 | 60 | NRCAM | Proximal | 0 | Not documented | 26 |
| AluY_dup49271 | Multi | No | — | — | SINE | Alu | chr7: 107828114-107828411 | + | 26 | 298 | NRCAM | Proximal | 0 | Not documented | 27 |
| MIR3_dup39819 | Multi | No | — | — | SINE | MIR | chr7: 107829599-107829743 | + | 150 | 145 | NRCAM | Proximal | 0 | Not documented | 28 |
| AluSx1_dup42974 | Uniquely | No | — | — | SINE | Alu | chr7: 107835786-107836073 | + | 70 | 288 | NRCAM | Proximal | 0 | Not documented | 29 |
| LTR85c_dup587 | Multi | No | — | — | LTR | Gypsy | chr7: 107847093-107847191 | + | 120 | 99 | NRCAM | Proximal | 0 | Not documented | 30 |
| AluY_dup49273 | Multi | No | — | — | SINE | Alu | chr7: 107855657-107855958 | − | 32 | 302 | NRCAM | Proximal | 0 | Not documented | 31 |
| L2c_dup64293 | Multi | No | — | — | LINE | L2 | chr7: 107881378-107881725 | + | 160 | 348 | NRCAM | Proximal | 0 | Not documented | 32 |
| L2b_dup42299 | Multi/ | No | — | — | LINE | L2 | chr7: 107881959-107882119 | + | 130 | 161 | NRCAM | Proximal | 0 | Not documented | 33 |
| | Uniquely | | | | | | | | | | | | | | |
| L4_dup8064 | Multi | No | — | — | LINE | RTE | chr7: 107892844-107892912 | + | 120 | 69 | NRCAM | Proximal | 0 | Not documented | 34 |
| L1PA14_dup1486 | Multi | No | — | — | LINE | L1PA\|B\|x | chr7: 107892954-107892993 | + | 11 | 40 | NRCAM | Proximal | 0 | Not documented | 35 |
| L1ME1_dup14200 | Multi | No | — | — | LINE | Other L1 | chr7: 107894487-107894989 | + | 87 | 503 | NRCAM | Proximal | 0 | Not documented | 36 |
| AluSx3_dup12091 | Multi | No | — | — | SINE | Alu | chr7: 107894990-107895293 | − | 94 | 304 | NRCAM | Proximal | 0 | Not documented | 37 |
| L2c_dup64297 | Multi | No | — | — | LINE | L2 | chr7: 107934949-107934987 | − | 70 | 39 | NRCAM | Proximal | 0 | Not documented | 38 |
| MIR_dup77560 | Multi | No | — | — | SINE | MIR | chr7: 107935630-107935767 | − | 150 | 138 | NRCAM | Proximal | 0 | Not documented | 39 |
| MLT1A0_dup9600 | Multi | No | — | — | LTR | ERVL- | chr7: 107935768-107936121 | − | 89 | 354 | NRCAM | Proximal | 0 | Not documented | 40 |
| | | | | | | MaLR | | | | | | | | | |
| MIR_dup77561 | Multi | No | — | — | SINE | MIR | chr7: 107936122-107936188 | − | 150 | 67 | NRCAM | Proximal | 0 | Not documented | 41 |
| L2a_dup77763 | Multi/ | No | — | — | LINE | L2 | chr7: 107938010-107938482 | − | 110 | 473 | NRCAM | Proximal | 0 | Not documented | 42 |
| | Uniquely | | | | | | | | | | | | | | |
| AluY_dup49274 | Multi/ | No | — | — | SINE | Alu | chr7: 107938809-107939128 | + | 40 | 320 | NRCAM | Proximal | 0 | Not documented | 43 |
| | Uniquely | | | | | | | | | | | | | | |
| AluSx_dup55714 | Multi/ | No | — | — | SINE | Alu | chr7: 107939270-107939577 | + | 49 | 308 | NRCAM | Proximal | 0 | Not documented | 44 |
| | Uniquely | | | | | | | | | | | | | | |
| L2a_dup77766 | Multi/ | No | — | — | LINE | L2 | chr7: 107965931-107966255 | + | 130 | 325 | NRCAM | Proximal | 0 | Not documented | 45 |
| | Uniquely | | | | | | | | | | | | | | |
| MIRc_dup45434 | Multi | No | — | — | SINE | MIR | chr7: 107976638-107976690 | − | 100 | 53 | NRCAM | Proximal | 0 | Not documented | 46 |
| MSTD_dup3639 | Multi/ | No | — | — | LTR | ERVL- | chr7: 107991480-107991810 | + | 75 | 331 | NRCAM | Proximal | 0 | Not documented | 47 |
| | Uniquely | | | | | MaLR | | | | | | | | | |
| MamGypLTR3_dup420 | Multi | No | — | — | LTR | Gypsy | chr7: 107991927-107992202 | + | 150 | 276 | NRCAM | Proximal | 0 | Not documented | 48 |
| LFSINE_Vert_{—} | Multi/ | No | — | — | SINE | SINE | chr7: 107992406-107992707 | − | 160 | 302 | NRCAM | Proximal | 0 | Not documented | 49 |
| dup484 | Uniquely | | | | | | | | | | | | | | |
| L1MA2_dup3012 | Multi | No | — | — | LINE | Other L1 | chr7: 108077234-108078028 | + | 44 | 795 | NRCAM | Proximal | 0 | Not documented | 3950 |
| MLT1C_dup8230 | Multi/ | No | — | — | LTR | ERVL- | chr7: 108084868-108085297 | − | 70 | 430 | NRCAM | Proximal | 0 | Not documented | 51 |
| | Uniquely | | | | | MaLR | | | | | | | | | |
| AluSx1_dup42979 | Multi/ | No | — | — | SINE | Alu | chr7: 108087177-108087468 | − | 55 | 292 | NRCAM | Proximal | 0 | Not documented | 52 |
| | Uniquely | | | | | | | | | | | | | | |
| AluSx3_dup12093 | Multi/ | No | — | — | SINE | Alu | chr7: 108087700-108088013 | − | 56 | 314 | NRCAM | Proximal | 0 | Not documented | 53 |
| | Uniquely | | | | | | | | | | | | | | |
| X7A_LINE_dup418 | Multi | No | — | — | LINE | CR1 | chr7: 108088385-108088455 | − | 100 | 71 | NRCAM | Proximal | 0 | Not documented | 54 |
| L2a_dup77777 | Multi | No | — | — | LINE | L2 | chr7: 108091233-108091272 | + | 100 | 40 | NRCAM | Proximal | 0 | Not documented | 55 |
| AluSx1_dup42980 | Multi | No | — | — | SINE | Alu | chr7: 108091381-108091692 | + | 63 | 312 | NRCAM | Proximal | 0 | Not documented | 56 |
| MIR_dup77569 | Multi/ | No | — | — | SINE | MIR | chr7: 108094823-108094996 | + | 110 | 174 | NRCAM | Proximal | 0 | Not documented | 57 |
| | Uniquely | | | | | | | | | | | | | | |
| MIRb_dup97492 | Multi | No | — | — | SINE | MIR | chr7: 108202972-108203131 | − | 150 | 160 | THAP5 | Proximal | 0 | Not documented | 58 |
| AluSx_dup55726 | Multi/ | No | — | — | SINE | Alu | chr7: 108203700-108204011 | + | 42 | 312 | THAP5 | Proximal | 0 | Not documented | 59 |
| | Uniquely | | | | | | | | | | | | | | |
| MLT1A0_dup9624 | Uniquely | No | — | — | LTR | ERVL- | chr7: 110828302-110828651 | − | 91 | 350 | IMMP2L | Proximal | 0 | Not documented | 60 |
| | | | | | | MaLR | | | | | | | | | |
| AluSz_dup38654 | Multi | No | — | — | SINE | Alu | chr7: 110906318-110906618 | + | 57 | 301 | IMMP2L | Proximal | 0 | Not documented | 61 |
| L1P4_dup1576 | Multi | No | — | — | LINE | L1PA\|B\|x | chr7: 110916639-110917495 | + | 65 | 857 | IMMP2L | Proximal | 0 | Not documented | 62 |
| L1MD1_dup2953 | Multi/ | No | — | — | LINE | Other L1 | chr7: 110940434-110941928 | − | 76 | 1495 | IMMP2L | Proximal | 0 | Not documented | 63 |
| | Uniquely | | | | | | | | | | | | | | |
| L1MC4_dup13044 | Multi/ | No | — | — | LINE | Other L1 | chr7: 110969239-110970729 | + | 120 | 1491 | IMMP2L | Proximal | 0 | Not documented | 64 |
| | Uniquely | | | | | | | | | | | | | | |
| L2_dup26139 | Uniquely | No | — | — | LINE | L2 | chr7: 111021906-111022881 | − | 170 | 976 | IMMP2L | Proximal | 0 | Not documented | 65 |
| L1PB_dup747 | Uniquely | No | — | — | LINE | L1PA\|B\|x | chr7: 111038231-111039422 | + | 35 | 1192 | IMMP2L | Proximal | 0 | EVE annotated | 66 |
| MSTB-int_dup370 | Uniquely | No | — | — | LTR | ERVL- | chr7: 11112716-11113826 | − | 88 | 1111 | PHF14 | Proximal | 0 | Not documented | 67 |
| | | | | | | MaLR | | | | | | | | | |
| AluJo_dup28391 | Multi | No | — | — | SINE | Alu | chr7: 112100297-112100598 | − | 72 | 302 | IFRD1 | Proximal | 0 | Not documented | 68 |
| MIR3_dup39920 | Multi | No | — | — | SINE | MIR | chr7: 112101587-112101787 | − | 170 | 201 | IFRD1 | Proximal | 0 | Not documented | 69 |
| L2b_dup42413 | Uniquely | No | — | — | LINE | L2 | chr7: 112428767-112429467 | + | 150 | 701 | TMEM168 | Proximal | 0 | Not documented | 70 |
| Tigger2_dup1338 | Uniquely | No | — | — | DNA | DNA | chr7: 116687926-116689977 | − | 60 | 2052 | ST7-OT4 | Proximal | 0 | Not documented | 71 |
| L2c_dup64777 | Multi | No | — | — | LINE | L2 | chr7: 116738282-116738692 | + | 150 | 411 | ST7-OT4 | Proximal | 0 | Not documented | 72 |
| Plat_L3_dup1727 | Multi | Nc | — | — | LINE | CR1 | chr7: 116774127-116774332 | − | 170 | 206 | ST7 | Proximal | 0 | Not documented | 73 |
| L1PA13_dup3710 | Uniquely | No | — | — | LINE | L1PA\|B\|x | chr7: 11986766-11990668 | − | 65 | 3903 | THSD7A | Distal | 114943 | Not documented | 74 |
| L2a_dup78468 | Uniquely | No | — | — | LINE | L2 | chr7: 119979652-119982001 | − | 140 | 2350 | KCND2 | Proximal | 0 | Not documented | 3975 |
| L2b_dup42588 | Uniquely | No | — | — | LINE | L2 | chr7: 120054845-120055360 | + | 160 | 516 | KCND2 | Proximal | 0 | Not documented | 76 |
| L2b_dup42589 | Uniquely | No | — | — | LINE | L2 | chr7: 120055526-120056241 | + | 160 | 716 | KCND2 | Proximal | 0 | Not documented | 77 |
| L1PA13_dup4004 | Uniquely | No | — | — | LINE | L1PA\|B\|x | chr7: 120092425-120093569 | + | 130 | 1145 | KCND2 | Proximal | 0 | Not documented | 78 |
| Tigger1_dup5804 | Uniquely | No | — | — | DNA | DNA | chr7: 120176110-120178489 | − | 64 | 2380 | KCND2 | Proximal | 0 | Not documented | 79 |
| LTR62_dup97 | Multi | No | — | — | LTR | ERVL | chr7: 1202508-1202802 | + | 100 | 295 | ZFANDZA | Distal | 2114 | Not documented | 80 |
| MLT2B3_dup1457 | Uniquely | No | — | — | LTR | ERVL | chr7: 1204112-1204643 | − | 110 | 532 | ZFANDZA | Distal | 3718 | Not documented | 81 |
| L1MB7_dup9209 | Multi/ | No | — | — | LINE | Other L1 | chr7: 121515110-121515485 | − | 73 | 376 | PTPRZ1 | Proximal | 0 | Not documented | 82 |
| | Uniquely | | | | | | | | | | | | | | |
| L2b_dup42636 | Multi | No | — | — | LINE | L2 | chr7: 121515942-121516013 | + | 160 | 72 | PTPRZ1 | Proximal | 0 | Not documented | 83 |
| MIRb_dup98195 | Multi/ | No | — | — | SINE | MIR | chr7: 121517137-121517308 | − | 150 | 172 | PTPRZ1 | Proximal | 0 | Not documented | 84 |
| | Uniquely | | | | | | | | | | | | | | |
| AluSc_dup14626 | Multi/ | No | — | — | SINE | Alu | chr7: 121556048-121556344 | + | 46 | 297 | PTPRZ1 | Proximal | 0 | Not documented | 85 |
| | Uniquely | | | | | | | | | | | | | | |
| MIRb_dup98198 | Multi/ | No | — | — | SINE | MIR | chr7: 121568488-121568673 | + | 100 | 186 | PTPRZ1 | Proximal | 0 | Not documented | 86 |
| | Uniquely | | | | | | | | | | | | | | |
| L1ME4a_dup19475 | Multi | No | — | — | LINE | Other L1 | chr7: 121569140-121569316 | − | 110 | 177 | PTPRZ1 | Proximal | 0 | Not documented | 87 |
| THE1D_dup5998 | Multi/ | No | — | — | LTR | ERVL- | chr7: 121618386-121618758 | − | 65 | 373 | PTPRZ1 | Proximal | 0 | Not documented | 88 |
| | Uniquely | | | | | MaLR | | | | | | | | | |
| AluYf4_dup582 | Multi | No | — | — | SINE | Alu | chr7: 121620915-121621221 | + | 22 | 307 | PTPRZ1 | Proximal | 0 | Not documented | 89 |
| L2b_dup42643 | Multi/ | No | — | — | LINE | L2 | chr7: 121625439-121626031 | + | 150 | 593 | PTPRZ1 | Proximal | 0 | Not documented | 90 |
| | Uniquely | | | | | | | | | | | | | | |
| MIRb_dup98200 | Multi/ | No | — | — | SINE | MIR | chr7: 121626194-121626423 | − | 130 | 230 | PTPRZ1 | Proximal | 0 | Not documented | 91 |
| | Uniquely | | | | | | | | | | | | | | |
| L1PA16_dup6424 | Multi | No | — | — | LINE | L1PA\|B\|x | chr7: 121627438-121627756 | − | 47 | 319 | PTPRZ1 | Proximal | 0 | Not documented | 92 |
| L1PA16_dup6425 | Multi | No | — | — | LINE | L1PA\|B\|x | chr7: 121627757-121628233 | + | 76 | 477 | PTPRZ1 | Proximal | 0 | Not documented | 93 |
| MIRb_dup98201 | Multi | No | — | — | SINE | MIR | chr7: 121631062-121631272 | + | 160 | 211 | PTPRZ1 | Proximal | 0 | Not documented | 94 |
| AluY_dup49612 | Multi | No | — | — | SINE | Alu | chr7: 121654509-121654791 | − | 29 | 283 | PTPRZ1 | Proximal | 0 | Not documented | 95 |
| AluSp_dup20064 | Multi | No | — | — | SINE | Alu | chr7: 121665371-121665684 | − | 42 | 314 | PTPRZ1 | Proximal | 0 | Not documented | 96 |
| L3_dup20726 | Multi | No | — | — | LINE | CR1 | chr7: 121666235-121666382 | + | 150 | 148 | PTPRZ1 | Proximal | 0 | Not documented | 97 |
| L2a_dup78566 | Multi/ | No | — | — | LINE | L2 | chr7: 121666928-121667490 | − | 150 | 563 | PTPRZ1 | Proximal | 0 | Not documented | 98 |
| | Uniquely | | | | | | | | | | | | | | |
| L1M3_dup2728 | Multi | No | — | — | LINE | Other L1 | chr7: 121668467-121668497 | + | 30 | 31 | PTPRZ1 | Proximal | 0 | Not documented | 99 |
| L1PA17_dup2142 | Multi | No | — | — | LINE | L1PA\|B\|x | chr7: 121669409-121669466 | − | 78 | 58 | PTPRZ1 | Proximal | 0 | Not documented | 4000 |
| MIRb_dup98202 | Multi/ | No | — | — | SINE | MIR | chr7: 121672282-121672493 | − | 150 | 212 | PTPRZ1 | Proximal | 0 | Not documented | 1 |
| | Uniquely | | | | | | | | | | | | | | |
| MIRc_dup45773 | Multi | No | — | — | SINE | MIR | chr7: 121679192-121679255 | + | 150 | 64 | PTPRZ1 | Proximal | 0 | Not documented | 2 |
| AluSz_dup38838 | Multi | No | — | — | SINE | Alu | chr7: 121679771-121680063 | + | 67 | 293 | PTPRZ1 | Proximal | 0 | Not documented | 3 |
| MLT1C_dup8325 | Uniquely | No | — | — | LTR | ERVL- | chr7: 121682860-121683268 | − | 100 | 409 | PTPRZ1 | Proximal | 0 | Not documented | 4 |
| | | | | | | MaLR | | | | | | | | | |
| AluJb_dup57765 | Multi/ | No | — | — | SINE | Alu | chr7: 121687702-121688007 | − | 76 | 306 | PTPRZ1 | Proximal | 0 | Not documented | 5 |
| | Uniquely | | | | | | | | | | | | | | |
| L1MB8_dup6735 | Multi/ | No | — | — | LINE | Other L1 | chr7: 121713629-121714148 | − | 79 | 520 | AASS | Proximal | 1554 | Not documented | 6 |
| | Uniquely | | | | | | | | | | | | | | |
| L1MB8_dup6736 | Multi/ | No | — | — | LINE | Other L1 | chr7: 121714149-121714875 | − | 79 | 727 | AASS | Proximal | 827 | Not documented | 7 |
| | Uniquely | | | | | | | | | | | | | | |
| AluJb_dup57767 | Multi | No | — | — | SINE | Alu | chr7: 121714904-121715076 | − | 100 | 173 | AASS | Proximal | 626 | Not documented | 8 |
| AluJb_dup57768 | Multi | No | — | — | SINE | Alu | chr7: 121715115-121715227 | − | 100 | 113 | AASS | Proximal | 475 | Not documented | 9 |
| L1MB8_dup6738 | Multi/ | No | — | — | LINE | Other L1 | chr7: 121715228-121715758 | − | 87 | 531 | AASS | Proximal | 0 | Not documented | 10 |
| | Uniquely | | | | | | | | | | | | | | |
| AluY_dup49614 | Multi/ | No | — | — | SINE | Alu | chr7: 121716028-121716257 | + | 22 | 230 | AASS | Proximal | 0 | Not documented | 11 |
| | Uniquely | | | | | | | | | | | | | | |
| MER44A_dup1137 | Multi/ | No | — | — | DNA | DNA | chr7: 121778306-121778649 | − | 59 | 344 | AASS | Proximal | 0 | Not documented | 12 |
| | Uniquely | | | | | | | | | | | | | | |
| AluSz6_dup18730 | Multi/ | No | — | — | SINE | Alu | chr7: 121779730-121779998 | + | 76 | 269 | AASS | Proximal | 0 | Not documented | 13 |
| | Uniquely | | | | | | | | | | | | | | |
| AluY_dup49615 | Multi/ | No | — | — | SINE | Alu | chr7: 121780258-121780565 | + | 28 | 308 | AASS | Proximal | 0 | Not documented | 14 |
| | Uniquely | | | | | | | | | | | | | | |
| AluSz_dup38931 | Multi | No | — | — | SINE | Alu | chr7: 127448215-127448518 | − | 64 | 304 | SND1 | Proximal | 0 | Not documented | 15 |
| L2b_dup42822 | Uniquely | No | — | — | LINE | L2 | chr7: 128096379-128096471 | + | 150 | 93 | HILPDA | Proximal | 0 | Not documented | 16 |
| AluSx1_dup43328 | Multi/ | No | — | — | SINE | Alu | chr7: 128097684-128097984 | + | 47 | 301 | HILPDA | Proximal | 0 | Not documented | 17 |
| | Uniquely | | | | | | | | | | | | | | |
| FLAM_A_dup7007 | Multi | No | — | — | SINE | Alu | chr7: 128142041-128142153 | + | 56 | 113 | METTL2B | Proximal | 0 | Not documented | 18 |
| AluSx1_dup43334 | Multi | No | — | — | SINE | Alu | chr7: 128142672-128142973 | + | 36 | 302 | METTL2B | Proximal | 0 | Not documented | 19 |
| AluSq_dup8742 | Multi | No | — | — | SINE | Alu | chr7: 128298962-128299089 | − | 53 | 128 | FAM71F2 | Distal | 13254 | Not documented | 20 |
| AluSx1_dup43355 | Multi | No | — | — | SINE | Alu | chr7: 128301151-128301461 | − | 57 | 311 | FAM71F2 | Distal | 10882 | Not documented | 21 |
| THE1B_dup10309 | Multi | No | — | — | LTR | ERVL- | chr7: 128301462-128301682 | + | 60 | 221 | FAM71F2 | Distal | 10661 | Not documented | 22 |
| | | | | | | MaLR | | | | | | | | | |
| THE1B-int_dup2153 | Multi/ | No | — | — | LTR | ERVL- | chr7: 128301683-128303228 | + | 36 | 1546 | FAM71F2 | Distal | 9115 | Not documented | 23 |
| | Uniquely | | | | | MaLR | | | | | | | | | |
| THE1B_dup10310 | Multi | No | — | — | LTR | ERVL- | chr7: 128303229-128303583 | + | 50 | 355 | FAM71F2 | Distal | 8760 | Not documented | 24 |
| | | | | | | MaLR | | | | | | | | | |
| AluJr_dup31250 | Multi | No | — | — | SINE | Alu | chr7: 128385392-128385667 | + | 82 | 276 | CALU | Proximal | 0 | Not documented | 4025 |
| L1ME3C_dup5509 | Multi | No | — | — | LINE | Other L1 | chr7: 128385898-128386205 | − | 130 | 308 | CALU | Proximal | 0 | Not documented | 26 |
| L1M5_dup28793 | Multi | No | — | — | LINE | Other L1 | chr7: 128624023-128624249 | − | 140 | 227 | TNPO3 | Proximal | 0 | Not documented | 27 |
| AluJb_dup58047 | Multi | No | — | — | SINE | Alu | chr7: 128921331-128921638 | − | 58 | 308 | AHCYL2 | Proximal | 0 | Not documented | 28 |
| L1ME1_dup14471 | Multi | No | — | — | LINE | Other L1 | chr7: 128921639-128922045 | − | 110 | 407 | AHCYL2 | Proximal | 0 | Not documented | 29 |
| L1ME1_dup14472 | Multi | No | — | — | LINE | Other L1 | chr7: 128922042-128923321 | − | 100 | 1280 | AHCYL2 | Proximal | 0 | Not documented | 30 |
| FLAM_A_dup7022 | Multi/ | No | — | — | SINE | Alu | chr7: 129069369-129069504 | − | 77 | 136 | AHCYL2 | Proximal | 0 | Not documented | 31 |
| | Uniquely | | | | | | | | | | | | | | |
| FLAM_C_dup9013 | Uniquely | No | — | — | SINE | Alu | chr7: 129250712-129250844 | + | 48 | 133 | NRF1 | Proximal | 712 | Not documented | 32 |
| L1MB8_dup6776 | Multi/ | No | — | — | LINE | Other L1 | chr7: 129494483-129495029 | + | 73 | 547 | UBE2H | Proximal | 0 | Not documented | 33 |
| | Uniquely | | | | | | | | | | | | | | |
| AluSx_dup56349 | Multi | No | — | — | SINE | Alu | chr7: 129805616-129805917 | + | 48 | 302 | TMEM209 | Proximal | 0 | Not documented | 34 |
| L3_dup20876 | Uniquely | No | — | — | LINE | CR1 | chr7: 130140460-130140586 | + | 130 | 127 | MEST | Proximal | 0 | Not documented | 35 |
| AluSx_dup56372 | Multi/ | No | — | — | SINE | Alu | chr7: 130141565-130141855 | + | 49 | 291 | MEST | Proximal | 0 | Not documented | 36 |
| | Uniquely | | | | | | | | | | | | | | |
| AluSp_dup20197 | Multi/ | No | — | — | SINE | Alu | chr7: 130151774-130152026 | + | 45 | 253 | COPG2 | Proximal | 0 | Not documented | 37 |
| | Uniquely | | | | | | | | | | | | | | |
| AluSz_dup39106 | Uniquely | No | — | — | SINE | Alu | chr7: 130152339-130152599 | + | 49 | 261 | COPG2 | Proximal | 0 | Not documented | 38 |
| AluSq2_dup22381 | Multi/ | No | — | — | SINE | Alu | chr7: 130152845-130153150 | + | 55 | 306 | COPG2 | Proximal | 0 | Not documented | 39 |
| | Uniquely | | | | | | | | | | | | | | |
| L1PA7_dup6349 | Uniquely | No | — | — | LINE | L1PA\|B\|x | chr7: 130317614-130324060 | − | 38 | 6447 | COPG2 | Proximal | 0 | EVE annotated | 40 |
| L1HS_dup780 | Multi | No | — | — | LINE | L1PA\|B\|x | chr7: 130576694-130577078 | + | 3.6 | 385 | KLF14 | Distal | 157807 | Not documented | 41 |
| MIR_dup78674 | Uniquely | No | — | — | SINE | MIR | chr7: 131015144-131015374 | − | 110 | 231 | MKLN1 | Proximal | 0 | Not documented | 42 |
| L1MEg_dup8462 | Multi | No | — | — | LINE | Other L1 | chr7: 131020326-131023378 | − | 110 | 3053 | MKLN1 | Proximal | 0 | Not documented | 43 |
| L1MEd_dup5131 | Multi | No | — | — | LINE | Other L1 | chr7: 131063661-131064255 | + | 150 | 595 | MKLN1 | Proximal | 0 | Not documented | 44 |
| L1MA1_dup1820 | Multi | No | — | — | LINE | Other L1 | chr7: 131064381-131064611 | + | 49 | 231 | MKLN1 | Proximal | 0 | Not documented | 45 |
| L1MEg_dup8464 | Multi | No | — | — | LINE | Other L1 | chr7: 131064612-131065055 | + | 150 | 444 | MKLN1 | Proximal | 0 | Not documented | 46 |
| AluJo_dup28726 | Multi | No | — | — | SINE | Alu | chr7: 131101456-131101762 | + | 79 | 307 | MKLN1 | Proximal | 0 | Not documented | 47 |
| MLT1H_dup4445 | Uniquely | No | — | — | LTR | ERVL- | chr7: 131116079-131116339 | − | 100 | 261 | MKLN1 | Proximal | 0 | Not documented | 48 |
| | | | | | | MaLR | | | | | | | | | |
| L1M5_dup28893 | Multi | No | — | — | LINE | Other L1 | chr7: 131138757-131139001 | + | 150 | 245 | MKLN1 | Proximal | 0 | Not documented | 49 |
| MLT1L_dup5697 | Multi | No | — | — | LTR | ERVL- | chr7: 131139288-131139805 | + | 120 | 518 | MKLN1 | Proximal | 0 | Not documented | 4050 |
| | | | | | | MaLR | | | | | | | | | |
| AluY_dup49983 | Multi | No | — | — | SINE | Alu | chr7: 131157428-131157734 | + | 31 | 307 | MKLN1 | Proximal | 0 | Not documented | 51 |
| L2a_dup79105 | Multi | No | — | — | LINE | L2 | chr7: 131158902-131159236 | − | 110 | 335 | MKLN1 | Proximal | 0 | Not documented | 52 |
| MSTA_dup8934 | Multi | No | — | — | LTR | ERVL- | chr7: 131159519-131159885 | − | 76 | 367 | MKLN1 | Proximal | 0 | Not documented | 53 |
| | | | | | | MaLR | | | | | | | | | |
| L1M5_dup28895 | Multi | No | — | — | LINE | Other L1 | chr7: 131159886-131160291 | + | 110 | 406 | MKLN1 | Proximal | 0 | Not documented | 54 |
| L1M5_dup28896 | Multi | No | — | — | LINE | Other L1 | chr7: 131160579-131162024 | + | 110 | 1446 | MKLN1 | Proximal | 0 | Not documented | 55 |
| AluJr_dup31413 | Multi | No | — | — | SINE | Alu | chr7: 131164013-131164301 | + | 74 | 289 | MKLN1 | Proximal | 0 | Not documented | 56 |
| AluY_dup49984 | Multi | No | — | — | SINE | Alu | chr7: 131164304-131164591 | + | 27 | 288 | MKLN1 | Proximal | 0 | Not documented | 57 |
| AluSx_dup56435 | Multi | No | — | — | SINE | Alu | chr7: 131164636-131164800 | + | 39 | 165 | MKLN1 | Proximal | 0 | Not documented | 58 |
| MIRb_dup98727 | Multi | No | — | — | SINE | MIR | chr7: 131165919-131166153 | + | 120 | 235 | MKLN1 | Proximal | 0 | Not documented | 59 |
| AluJo_dup28730 | Multi | No | — | — | SINE | Alu | chr7: 131166180-131166297 | + | 110 | 118 | MKLN1 | Proximal | 0 | Not documented | 60 |
| L1MC4_dup13259 | Uniquely | No | — | — | LINE | Other L1 | chr7: 131169441-131170845 | + | 98 | 1405 | MKLN1 | Proximal | 0 | Not documented | 61 |
| AluJr_dup31414 | Multi | No | — | — | SINE | Alu | chr7: 131177209-131177515 | + | 67 | 307 | MKLN1 | Proximal | 0 | Not documented | 62 |
| MIR_dup78756 | Uniquely | No | — | — | SINE | MIR | chr7: 132151077-132151310 | + | 150 | 234 | PLXNA4 | Proximal | 0 | Not documented | 63 |
| AluSg4_dup3053 | Uniquely | No | — | — | SINE | Alu | chr7: 132659354-132659663 | + | 41 | 310 | CHCHD3 | Proximal | 0 | Not documented | 64 |
| MIR_dup78835 | Multi | No | — | — | SINE | MIR | chr7: 133343929-133344057 | + | 170 | 129 | EXOC4 | Proximal | 0 | Not documented | 65 |
| AluSz_dup39247 | Multi | No | — | — | SINE | Alu | chr7: 133978541-133978843 | − | 48 | 303 | SLC35B4 | Proximal | 0 | Not documented | 66 |
| X6A_LINE_dup135 | Multi | No | — | — | LINE | CR1 | chr7: 133991446-133991489 | + | 110 | 44 | SLC35B4 | Proximal | 0 | Not documented | 67 |
| L2a_dup79335 | Multi/ | No | — | — | LINE | L2 | chr7: 134364094-134364145 | − | 79 | 52 | BPGM | Proximal | 0 | Not documented | 68 |
| | Uniquely | | | | | | | | | | | | | | |
| L1MA5_dup2078 | Multi | No | — | — | LINE | Other L1 | chr7: 134768966-134776042 | − | 73 | 7077 | AGBL3 | Proximal | 0 | Not documented | 69 |
| L1M4b_dup2752 | Uniquely | No | — | — | LINE | Other L1 | chr7: 134828423-134829351 | + | 100 | 929 | C7orf49 | Proximal | 0 | Not documented | 70 |
| Harlequin- | Multi | No | — | — | LTR | ERV1 | chr7: 134856495-134856835 | + | 46 | 341 | C7orf49 | Proximal | 949 | Not documented | 71 |
| int_dup266 | | | | | | | | | | | | | | | |
| MER6_dup380 | Multi | No | — | — | DNA | DNA | chr7: 135093233-135093621 | − | 80 | 389 | CNOT4 | Proximal | 0 | Not documented | 72 |
| L1ME4a_dup19746 | Multi | No | — | — | LINE | Other L1 | chr7: 135093647-135093752 | + | 130 | 106 | CNOT4 | Proximal | 0 | Not documented | 73 |
| L1MC4a_dup12329 | Multi | No | — | — | LINE | Other L1 | chr7: 135360136-135360353 | − | 130 | 218 | C7orf73 | Proximal | 0 | Not documented | 74 |
| AluSz6_dup18965 | Multi | No | — | — | SINE | Alu | chr7: 135360671-135360960 | − | 58 | 290 | C7orf73 | Proximal | 0 | Not documented | 4075 |
| MIRb_dup99051 | Multi | No | — | — | SINE | MIR | chr7: 135361016-135361153 | + | 120 | 138 | C7orf73 | Proximal | 0 | Not documented | 76 |
| ERV3-16A3_I- | Uniquely | No | — | — | LTR | ERVL | chr7: 136892411-136893619 | − | 120 | 1209 | PTN | Distal | 18470 | Not documented | 77 |
| int_dup3152 | | | | | | | | | | | | | | | |
| Harlequin- | Uniquely | No | — | — | LTR | ERV1 | chr7: 136947745-136950633 | − | 38 | 2889 | PTN | Proximal | 0 | EVE annotated | 78 |
| int_dup271 | | | | | | | | | | | | | | | |
| Harlequin- | Multi/ | No | — | — | LTR | ERV1 | chr7: 136951623-136952708 | − | 30 | 1086 | PTN | Proximal | 0 | EVE annotated | 79 |
| int_dup273 | Uniquely | | | | | | | | | | | | | | |
| Tigger3a_dup2593 | Multi | No | — | — | DNA | DNA | chr7: 137198285-137198563 | − | 54 | 279 | DGKI | Proximal | 0 | Not documented | 80 |
| AluSx_dup56719 | Multi | No | — | — | SINE | Alu | chr7: 138151209-138151397 | − | 72 | 189 | TRIM24 | Proximal | 0 | Not documented | 81 |
| L1MEc_dup8768 | Multi | No | — | — | LINE | Other L1 | chr7: 138151402-138152072 | − | 110 | 671 | TRIM24 | Proximal | 0 | Not documented | 82 |
| AluSc_dup14798 | Multi/ | No | — | — | SINE | Alu | chr7: 138152073-138152386 | − | 40 | 314 | TRIM24 | Proximal | 0 | Not documented | 83 |
| | Uniquely | | | | | | | | | | | | | | |
| L1MEc_dup8769 | Multi | No | — | — | LINE | Other L1 | chr7: 138152387-138153095 | − | 110 | 709 | TRIM24 | Proximal | 0 | Not documented | 84 |
| L1MEc_dup8770 | Multi | No | — | — | LINE | Other L1 | chr7: 138153122-138153418 | − | 110 | 297 | TRIM24 | Proximal | 0 | Not documented | 85 |
| L1PA6_dup2978 | Uniquely | No | — | — | LINE | L1PA\|LxB | chr7: 138170099-138175203 | − | 30 | 5105 | TRIM24 | Proximal | 0 | EVE annotated | 86 |
| L1MEg_dup8489 | Uniquely | No | — | — | LINE | Other L1 | chr7: 138204706-138205251 | + | 140 | 546 | TRIM24 | Proximal | 0 | Not documented | 87 |
| AluJo_dup28934 | Multi | No | — | — | SINE | Alu | chr7: 138707008-138707297 | + | 75 | 290 | ZC3HAV1L | Distal | 3156 | Not documented | 88 |
| MER5B_dup11312 | Multi | No | — | — | DNA | DNA | chr7: 138731749-138731791 | − | 81 | 43 | ZC3HAV1 | Proximal | 0 | Not documented | 89 |
| AluSx3_dup12342 | Multi | No | — | — | SINE | Alu | chr7: 138731792-138732097 | + | 46 | 306 | ZC3HAV1 | Proximal | 0 | Not documented | 90 |
| MER5B_dup11313 | Multi | No | — | — | DNA | DNA | chr7: 138732098-138732219 | − | 81 | 122 | ZC3HAV1 | Proximal | 0 | Not documented | 91 |
| AluJb_dup51472 | Uniquely | No | — | — | SINE | Alu | chr7: 139067127-139067438 | − | 83 | 312 | LUC7L2 | Proximal | 0 | Not documented | 92 |
| MER103C_dup4314 | Multi | No | — | — | DNA | DNA | chr7: 139102791-139102887 | − | 100 | 97 | LUC7L2 | Proximal | 0 | Not documented | 93 |
| AluSz_dup39464 | Multi | No | — | — | SINE | Alu | chr7: 139103582-139103887 | + | 49 | 306 | LUC7L2 | Proximal | 0 | Not documented | 94 |
| SVA_D_dup612 | Multi | No | — | — | Other | SVA | chr7: 139114123-139114720 | − | 10 | 598 | C7orf55-LUC7L2 | Distal | 5926 | Not documented | 95 |
| MIRb_dup99271 | Multi | No | — | — | SINE | MIR | chr7: 139250989-139251213 | + | 140 | 225 | HIPK2 | Proximal | 0 | Not documented | 96 |
| MIRb_dup99272 | Multi/ | No | — | — | SINE | MIR | chr7: 139251360-139251511 | + | 150 | 152 | HIPK2 | Proximal | 0 | Not documented | 97 |
| | Uniquely | | | | | | | | | | | | | | |
| Tigger5_dup1210 | Multi/ | No | — | — | DNA | DNA | chr7: 140033998-140034214 | + | 67 | 217 | SLC37A3 | Proximal | 0 | Not documented | 98 |
| | Uniquely | | | | | | | | | | | | | | |
| L1MB7_dup9369 | Uniquely | No | — | — | LINE | Other L1 | chr7: 140247195-140247893 | + | 72 | 699 | DENND2A | Proximal | 0 | Not documented | 99 |
| L2c_dup65999 | Uniquely | No | — | — | LINE | L2 | chr7: 141052815-141052986 | + | 150 | 172 | TMEM178B | Proximal | 0 | Not documented | 4100 |
| MIRb_dup99369 | Uniquely | No | — | — | SINE | MIR | chr7: 141094196-141094362 | + | 120 | 167 | TMEM178B | Proximal | 0 | Not documented | 1 |
| MER5A_dup16192 | Multi | No | — | — | DNA | DNA | chr7: 141353301-141353451 | − | 120 | 151 | AGK | Proximal | 0 | Not documented | 2 |
| Charlie2b_dup1790 | Multi | No | — | — | DNA | DNA | chr7: 141353495-141354402 | − | 95 | 908 | AGK | Proximal | 0 | Not documented | 3 |
| L1ME4a_dup19901 | Multi | No | — | — | LINE | Other L1 | chr7: 142967284-142967433 | + | 120 | 150 | GSTK1 | Proximal | 0 | Not documented | 4 |
| AluSq2_dup22658 | Multi | No | — | — | SINE | Alu | chr7: 142967434-142967725 | + | 40 | 292 | GSTK1 | Proximal | 0 | Not documented | 5 |
| L1MD2_dup4718 | Multi/ | No | — | — | LINE | Other L1 | chr7: 142968989-142969680 | − | 80 | 692 | GSTK1 | Proximal | 1043 | Not documented | 6 |
| | Uniquely | | | | | | | | | | | | | | |
| AluY_dup50417 | Multi | No | — | — | SINE | Alu | chr7: 143003343-143003647 | − | 31 | 305 | CASP2 | Proximal | 0 | Not documented | 7 |
| FLAM_C_dup9143 | Multi | No | — | — | SINE | Alu | chr7: 143003760-143003888 | − | 74 | 129 | CASP2 | Proximal | 0 | Not documented | 8 |
| L2a_dup79768 | Multi | No | — | — | LINE | L2 | chr7: 143003901-143004409 | + | 130 | 509 | CASP2 | Proximal | 0 | Not documented | 9 |
| L2c_dup66107 | Multi | No | — | — | LINE | L2 | chr7: 143004470-143004644 | + | 120 | 175 | CASP2 | Proximal | 0 | Not documented | 10 |
| MIR3_dup40795 | Multi | No | — | — | SINE | MIR | chr7: 143076993-143077103 | + | 130 | 111 | ZYX | Proximal | 1071 | Not documented | 11 |
| HAL1_dup13103 | Multi | No | — | — | LINE | Other L1 | chr7: 143304831-143305012 | + | 160 | 182 | FAM115C | Distal | 13032 | Not documented | 12 |
| AluJr_dup31757 | Multi | No | — | — | SINE | Alu | chr7: 143305714-143305952 | + | 65 | 239 | FAM115C | Distal | 12092 | Not documented | 13 |
| HAL1_dup13104 | Multi | No | — | — | LINE | Other L1 | chr7: 143305957-143306638 | + | 120 | 682 | FAM115C | Distal | 11406 | Not documented | 14 |
| HAL1_dup13105 | Multi | No | — | — | LINE | Other L1 | chr7: 143307376-143307635 | + | 110 | 260 | FAM115C | Distal | 10409 | Not documented | 15 |
| LTR45C_dup173 | Multi | No | — | — | LTR | ERV1 | chr7: 143342468-143342853 | + | 72 | 386 | FAM115C | Proximal | 0 | Not documented | 16 |
| LTR45C_dup174 | Multi | No | — | — | LTR | ERV1 | chr7: 143418662-143419055 | − | 73 | 394 | FAM115C | Proximal | 0 | Not documented | 17 |
| LTR45C_dup175 | Multi | No | — | — | LTR | ERV1 | chr7: 143508970-143509361 | − | 72 | 392 | FAM115A | Distal | 39108 | Not documented | 18 |
| HAL1_dup13109 | Multi | No | — | — | LINE | Other L1 | chr7: 143548494-143548749 | − | 100 | 256 | FAM115A | Proximal | 0 | Not documented | 19 |
| HAL1_dup13110 | Multi | No | — | — | LINE | Other L1 | chr7: 143549487-143550164 | − | 120 | 678 | FAM115A | Proximal | 0 | Not documented | 20 |
| AluJr_dup31758 | Multi | No | — | — | SINE | Alu | chr7: 143550169-143550407 | − | 63 | 239 | FAM115A | Proximal | 0 | Not documented | 21 |
| HAL1_dup13111 | Multi | No | — | — | LINE | Other L1 | chr7: 143551209-143551290 | − | 150 | 82 | FAM115A | Proximal | 0 | Not documented | 22 |
| AluSc8_dup9193 | Uniquely | No | — | — | SINE | Alu | chr7: 148199204-148199472 | + | 40 | 269 | CNTNAP2 | Distal | 81115 | Not documented | 23 |
| Tigger15a_dup2518 | Multi/ | No | — | — | DNA | DNA | chr7: 148522692-148523022 | + | 140 | 331 | EZH2 | Proximal | 0 | Not documented | 24 |
| | Uniquely | | | | | | | | | | | | | | |
| MIR_dup79576 | Multi/ | No | — | — | SINE | MIR | chr7: 148552471-148552680 | + | 110 | 210 | EZH2 | Proximal | 0 | Not documented | 4125 |
| | Uniquely | | | | | | | | | | | | | | |
| AluSg_dup17340 | Multi/ | No | — | — | SINE | Alu | chr7: 148552714-148553013 | + | 35 | 300 | EZH2 | Proximal | 0 | Not documented | 26 |
| | Uniquely | | | | | | | | | | | | | | |
| L1M4c_dup2472 | Multi | No | — | — | LINE | Other L1 | chr7: 148700502-148700781 | + | 140 | 280 | PDIA4 | Proximal | 0 | Not documented | 27 |
| AluJo_dup29140 | Uniquely | No | — | — | SINE | Alu | chr7: 148800312-148800606 | − | 59 | 295 | ZNF425 | Proximal | 0 | Not documented | 28 |
| L3_dup21304 | Uniquely | No | — | — | LINE | CR1 | chr7: 148879054-148879178 | + | 130 | 125 | ZNF398 | Proximal | 0 | Not documented | 29 |
| MIR3_dup40951 | Multi | No | — | — | SINE | MIR | chr7: 148981959-148981996 | + | 110 | 38 | ZNF783 | Proximal | 0 | Not documented | 30 |
| L2b_dup43409 | Multi | No | — | — | LINE | L2 | chr7: 148994309-148994370 | + | 120 | 62 | ZNF783 | Proximal | 0 | Not documented | 31 |
| MIRc_dup46623 | Multi | No | — | — | SINE | MIR | chr7: 149170486-149170565 | + | 96 | 80 | ZNF746 | Proximal | 0 | Not documented | 32 |
| MER70B_dup226 | Multi | No | — | — | LTR | ERVL | chr7: 149244195-149244631 | − | 86 | 437 | ZNF746 | Distal | 49288 | Not documented | 33 |
| LOR1a_dup376 | Uniquely | No | — | — | LTR | ERV1 | chr7: 149245146-149245645 | + | 66 | 500 | ZNF746 | Distal | 50239 | Not documented | 34 |
| L2c_dup66365 | Multi | No | — | — | LINE | L2 | chr7: 149536532-149536627 | − | 160 | 96 | ZNF862 | Proximal | 0 | Not documented | 35 |
| Tigger3_dup570 | Multi | No | — | — | DNA | DNA | chr7: 149537100-149537200 | + | 110 | 101 | ZNF862 | Proximal | 0 | Not documented | 36 |
| L3_dup21315 | Multi/ | No | — | — | LINE | CR1 | chr7: 150070867-150071095 | − | 140 | 229 | REPIN1 | Proximal | 0 | Not documented | 37 |
| | Uniquely | | | | | | | | | | | | | | |
| MIR3_dup41007 | Multi/ | No | — | — | SINE | MIR | chr7: 150774485-150774599 | + | 170 | 115 | FASTK | Proximal | 0 | Not documented | 38 |
| | Uniquely | | | | | | | | | | | | | | |
| MIR_dup79654 | Multi | No | — | — | SINE | MIR | chr7: 150937804-150938054 | + | 120 | 251 | SMARCD3 | Proximal | 0 | Not documented | 39 |
| MIRc_dup46667 | Multi | No | — | — | SINE | MIR | chr7: 150938099-150938296 | − | 140 | 198 | SMARCD3 | Proximal | 0 | Not documented | 40 |
| MIRc_dup46668 | Multi | No | — | — | SINE | MIR | chr7: 150938356-150938474 | − | 140 | 119 | SMARCD3 | Proximal | 0 | Not documented | 41 |
| L1MC5_dup9255 | Multi | No | — | — | LINE | Other L1 | chr7: 151057646-151057711 | − | 110 | 66 | NUB1 | Proximal | 0 | Not documented | 42 |
| AluJb_dup59167 | Multi | No | — | — | SINE | Alu | chr7: 151057712-151058017 | − | 70 | 306 | NUB1 | Proximal | 0 | Not documented | 43 |
| L1MC5_dup9256 | Multi | No | — | — | LINE | Other L1 | chr7: 151058167-151058624 | − | 110 | 458 | NUB1 | Proximal | 0 | Not documented | 44 |
| MIR_dup79693 | Multi | No | — | — | SINE | MIR | chr7: 151903091-151903275 | + | 120 | 185 | KMT2C | Proximal | 0 | Not documented | 45 |
| L1MEc_dup8884 | Uniquely | No | — | — | LINE | Other L1 | chr7: 151924463-151924850 | + | 140 | 388 | KMT2C | Proximal | 0 | Not documented | 46 |
| L1MDa_dup3081 | Multi | No | — | — | LINE | Other L1 | chr7: 151983680-151984827 | + | 120 | 1148 | KMT2C | Proximal | 0 | Not documented | 47 |
| AluSz_dup39963 | Multi | No | — | — | SINE | Alu | chr7: 151984828-151985130 | + | 54 | 303 | KMT2C | Proximal | 0 | Not documented | 48 |
| L1MEc_dup8886 | Multi | No | — | — | LINE | Other L1 | chr7: 151994223-151996122 | + | 95 | 1900 | KMT2C | Proximal | 0 | Not documented | 49 |
| AluSx_dup57585 | Multi | No | — | — | SINE | Alu | chr7: 152290963-152291265 | − | 40 | 303 | XRCC2 | Distal | 50600 | Not documented | 4150 |
| L1ME1_dup14715 | Uniquely | No | — | — | LINE | Other L1 | chr7: 152539652-152540590 | − | 110 | 939 | ACTR3B | Proximal | 0 | Not documented | 51 |
| AluSq2_dup22884 | Uniquely | No | — | — | SINE | Alu | chr7: 153488506-153488803 | − | 52 | 298 | DPP6 | Distal | 95380 | Not documented | 52 |
| L1ME3A_dup7328 | Uniquely | No | — | — | LINE | Other L1 | chr7: 153574913-153575870 | + | 120 | 958 | DPP6 | Distal | 8313 | Not documented | 53 |
| AluSz_dup40045 | Multi/ | No | — | — | SINE | Alu | chr7: 153711471-153711776 | + | 49 | 306 | DPP6 | Proximal | 0 | Not documented | 54 |
| | Uniquely | | | | | | | | | | | | | | |
| L2a_dup80351 | Uniquely | No | — | — | LINE | L2 | chr7: 153712248-153713321 | + | 140 | 1074 | DPP6 | Proximal | 0 | Not documented | 55 |
| MER103C_dup4361 | Uniquely | No | — | — | DNA | DNA | chr7: 153828246-153828448 | + | 170 | 203 | DPP6 | Proximal | 0 | Not documented | 56 |
| L1ME1_dup14739 | Uniquely | No | — | — | LINE | Other L1 | chr7: 153891729-153892331 | − | 110 | 603 | DPP6 | Proximal | 0 | Not documented | 57 |
| MIR_dup79768 | Uniquely | No | — | — | SINE | MIR | chr7: 153907461-153907659 | + | 150 | 199 | DPP6 | Proximal | 0 | Not documented | 58 |
| Tigger2a_dup1603 | Uniquely | No | — | — | DNA | DNA | chr7: 153936389-153936767 | + | 58 | 379 | DPP6 | Proximal | 0 | Not documented | 59 |
| L1ME2_dup5946 | Uniquely | No | — | — | LINE | Other L1 | chr7: 153939540-153940597 | + | 130 | 1058 | DPP6 | Proximal | 0 | Not documented | 60 |
| LTR87_dup321 | Uniquely | No | — | — | LTR | ERVL | chr7: 154501625-154501912 | + | 120 | 288 | DPP6 | Proximal | 0 | Not documented | 61 |
| AluSx3_dup10918 | Multi | No | — | — | SINE | Alu | chr7: 154796010-154796310 | + | 38 | 301 | PAXIP1 | Proximal | 1217 | Not documented | 62 |
| L1MD1_dup2750 | Uniquely | No | — | — | LINE | Other L1 | chr7: 15692293-15698196 | − | 93 | 5904 | MEOX2 | Proximal | 0 | Not documented | 63 |
| L2_dup27139 | Uniquely | No | — | — | LINE | L2 | chr7: 158725212-158725800 | − | 170 | 589 | WDR60 | Proximal | 0 | Not documented | 64 |
| AluJr_dup26599 | Multi | No | — | — | SINE | Alu | chr7: 1609122-1609418 | + | 67 | 297 | PSMG3 | Proximal | 0 | Not documented | 65 |
| L2a_dup73623 | Uniquely | No | — | — | LINE | L2 | chr7: 20384453-20384789 | − | 140 | 337 | ITGB8 | Proximal | 0 | Not documented | 66 |
| MLT1J_dup6601 | Uniquely | No | — | — | LTR | ERVL- | chr7: 20394065-20394481 | − | 150 | 417 | ITGB8 | Proximal | 0 | Not documented | 67 |
| | | | | | | MaLR | | | | | | | | | |
| MIRb_dup92056 | Uniquely | No | — | — | SINE | MIR | chr7: 20404977-20405201 | + | 130 | 225 | ITGB8 | Proximal | 0 | Not documented | 68 |
| MLT1A_dup3909 | Uniquely | No | — | — | LTR | ERVL- | chr7: 20408877-20409269 | − | 85 | 393 | ITGB8 | Proximal | 0 | Not documented | 69 |
| | | | | | | MaLR | | | | | | | | | |
| AluSq2_dup20161 | Multi/ | No | — | — | SINE | Alu | chr7: 20450770-20451076 | − | 49 | 307 | ITGB8 | Proximal | 0 | Not documented | 70 |
| | Uniquely | | | | | | | | | | | | | | |
| MIRc_dup42961 | Multi/ | No | — | — | SINE | MIR | chr7: 20451077-20451232 | − | 180 | 156 | ITGB8 | Proximal | 0 | Not documented | 71 |
| | Uniquely | | | | | | | | | | | | | | |
| MER91C_dup630 | Multi/ | No | — | — | DNA | DNA | chr7: 20451398-20451479 | + | 160 | 82 | ITGB8 | Proximal | 0 | Not documented | 72 |
| | Uniquely | | | | | | | | | | | | | | |
| Kangala_dup367 | Multi | No | — | — | DNA | DNA | chr7: 20453649-20453724 | + | 96 | 76 | ITGB8 | Proximal | 0 | Not documented | 73 |
| AluSx1_dup38682 | Multi | No | — | — | SINE | Alu | chr7: 21552181-21552479 | − | 38 | 299 | SP4 | Proximal | 0 | Not documented | 74 |
| AluSx_dup50421 | Multi | No | — | — | SINE | Alu | chr7: 21941047-21941198 | + | 66 | 152 | CDCA7L | Proximal | 0 | Not documented | 4175 |
| AluJr4_dup6915 | Multi | No | — | — | SINE | Alu | chr7: 2274157-2274289 | − | 89 | 133 | FTSJ2 | Proximal | 0 | Not documented | 76 |
| AluY_dup45580 | Uniquely | No | — | — | SINE | Alu | chr7: 23161300-23161599 | − | 44 | 300 | KLHL7 | Proximal | 0 | Not documented | 77 |
| MER44B_dup947 | Multi | No | — | — | DNA | DNA | chr7: 23165480-23165990 | − | 63 | 511 | KLHL7 | Proximal | 0 | Not documented | 78 |
| X3_LINE_dup333 | Multi/ | No | — | — | LINE | RTE-BovB | chr7: 2402314-2402363 | + | 120 | 50 | EIF3B | Proximal | 0 | Not documented | 79 |
| | Uniquely | | | | | | | | | | | | | | |
| L1PA7_dup5882 | Uniquely | No | — | — | LINE | L1PA\|B\|x | chr7: 24666089-24670119 | + | 37 | 4031 | MPP6 | Proximal | 0 | EVE annotated | 80 |
| L2a_dup73965 | Multi/ | No | — | — | LINE | L2 | chr7: 26213888-26214209 | − | 120 | 322 | NFE2L3 | Proximal | 0 | Not documented | 81 |
| | Uniquely | | | | | | | | | | | | | | |
| AluY_dup45699 | Multi/ | No | — | — | SINE | Alu | chr7: 26214685-26214992 | + | 35 | 308 | NFE2L3 | Proximal | 0 | Not documented | 82 |
| | Uniquely | | | | | | | | | | | | | | |
| AluSc8_dup8232 | Uniquely | No | — | — | SINE | Alu | chr7: 26442990-26443260 | − | 44 | 271 | SNX10 | Distal | 29042 | Not documented | 83 |
| MIRc_dup43146 | Multi/ | No | — | — | SINE | MIR | chr7: 26534242-26534276 | − | 78 | 35 | KIAA0087 | Distal | 38465 | Not documented | 84 |
| | Uniquely | | | | | | | | | | | | | | |
| L2a_dup73979 | Multi | No | — | — | LINE | L2 | chr7: 26534305-26534499 | − | 120 | 195 | KIAA0087 | Distal | 38242 | Not documented | 85 |
| Tigger2_dup1241 | Uniquely | No | — | — | DNA | DNA | chr7: 27579272-27581923 | − | 52 | 2652 | HIBADH | Proximal | 0 | Not documented | 86 |
| MIR3_dup37254 | Multi/ | No | — | — | SINE | MIR | chr7: 2768410-2768528 | − | 170 | 119 | AMZ1 | Proximal | 0 | Not documented | 87 |
| | Uniquely | | | | | | | | | | | | | | |
| MLT1F2_dup2510 | Uniquely | No | — | — | LTR | ERVL- | chr7: 28470344-28470920 | − | 110 | 577 | CREB5 | Proximal | 0 | Not documented | 88 |
| | | | | | | MaLR | | | | | | | | | |
| Charlie19a_dup668 | Uniquely | No | — | — | DNA | DNA | chr7: 28478074-28478388 | − | 110 | 315 | CREB5 | Proximal | 0 | Not documented | 89 |
| L2b_dup40345 | Uniquely | No | — | — | LINE | L2 | chr7: 28478921-28479057 | + | 130 | 137 | CREB5 | Proximal | 0 | Not documented | 90 |
| MIR_dup73671 | Uniquely | No | — | — | SINE | MIR | chr7: 28479106-28479309 | − | 110 | 204 | CREB5 | Proximal | 0 | Not documented | 91 |
| AluSx1_dup38936 | Multi | No | — | — | SINE | Alu | chr7: 28730755-28731058 | + | 51 | 304 | CREB5 | Proximal | 0 | Not documented | 92 |
| AluSx3_dup11211 | Multi | No | — | — | SINE | Alu | chr7: 28731504-28731815 | + | 47 | 312 | CREB5 | Proximal | 0 | Not documented | 93 |
| AluSq2_dup20309 | Uniquely | No | — | — | SINE | Alu | chr7: 28745691-28745986 | + | 52 | 296 | CREB5 | Proximal | 0 | Not documented | 94 |
| MIR3_dup37900 | Uniquely | No | — | — | SINE | MIR | chr7: 28771081-28771273 | − | 130 | 193 | CREB5 | Proximal | 0 | Not documented | 95 |
| L2c_dup61157 | Multi/ | No | — | — | LINE | L2 | chr7: 28772992-28773424 | + | 130 | 433 | CREB5 | Proximal | 0 | Not documented | 96 |
| | Uniquely | | | | | | | | | | | | | | |
| L1MB7_dup8503 | Uniquely | No | — | — | LINE | Other L1 | chr7: 28816158-28816769 | − | 74 | 612 | CREB5 | Proximal | 0 | Not documented | 97 |
| AluSg4_dup2784 | Uniquely | No | — | — | SINE | Alu | chr7: 28846784-28847080 | + | 38 | 297 | CREB5 | Proximal | 0 | Not documented | 98 |
| MIRb_dup92681 | Uniquely | No | — | — | SINE | MIR | chr7: 28849272-28849491 | + | 130 | 220 | CREB5 | Proximal | 0 | Not documented | 99 |
| MER20_dup6494 | Uniquely | No | — | — | DNA | DNA | chr7: 28851560-28851786 | − | 78 | 227 | CREB5 | Proximal | 0 | Not documented | 4200 |
| L1MB3_dup6545 | Uniquely | No | — | — | LINE | Other L1 | chr7: 29949845-29952142 | + | 78 | 2298 | WIPF3 | Proximal | 0 | EVE annotated | 1 |
| AluSp_dup18604 | Uniquely | No | — | — | SINE | Alu | chr7: 30053501-30053777 | − | 41 | 277 | FKBP14 | Proximal | 0 | Not documented | 2 |
| PABL_B_dup144 | Uniquely | No | — | — | LTR | ERV1 | chr7: 30605583-30605994 | − | 49 | 412 | GGCT | Distal | 14489 | Not documented | 3 |
| MER4-int_dup1104 | Multi | No | — | — | LTR | ERV1 | chr7: 30608987-30609663 | + | 78 | 677 | GGCT | Distal | 17893 | Not documented | 4 |
| LTR12_dup333 | Multi/ | No | — | — | LTR | ERV1 | chr7: 30609668-30610518 | + | 40 | 851 | GGCT | Distal | 18574 | Not documented | 5 |
| | Uniquely | | | | | | | | | | | | | | |
| AluSx_dup50797 | Multi | No | — | — | SINE | Alu | chr7: 30624815-30625118 | + | 57 | 304 | GARS | Distal | 9180 | Not documented | 6 |
| MIR3_dup38023 | Uniquely | No | — | — | SINE | MIR | chr7: 30964787-30964831 | − | 62 | 45 | AQP1 | Proximal | 0 | Not documented | 7 |
| MIR3_dup38048 | Multi/ | No | — | — | SINE | MIR | chr7: 31148119-31148248 | + | 140 | 130 | ADCYAP1R1 | Proximal | 0 | Not documented | 8 |
| | Uniquely | | | | | | | | | | | | | | |
| MIR3_dup38050 | Multi | No | — | — | SINE | MIR | chr7: 31150008-31150080 | − | 120 | 73 | ADCYAP1R1 | Proximal | 0 | Not documented | 9 |
| MIRc_dup43351 | Multi/ | No | — | — | SINE | MIR | chr7: 31150277-31150508 | + | 160 | 232 | ADCYAP1R1 | Proximal | 0 | Not documented | 10 |
| | Uniquely | | | | | | | | | | | | | | |
| HERVH-int_dup2862 | Uniquely | No | — | — | LTR | ERV1 | chr7: 32872632-32876224 | + | 46 | 3593 | AVL9 | Proximal | 0 | EVE annotated | 11 |
| AluYc_dup3297 | Multi | No | — | — | SINE | Alu | chr7: 32928243-32928325 | + | 22 | 83 | KBTBD2 | Proximal | 0 | Not documented | 12 |
| AluY_dup45920 | Uniquely | No | — | — | SINE | Alu | chr7: 32928643-32928920 | + | 25 | 278 | KBTBD2 | Proximal | 0 | Not documented | 13 |
| AluSg_dup15640 | Uniquely | No | — | — | SINE | Alu | chr7: 32928933-32929218 | + | 45 | 286 | KBTBD2 | Proximal | 0 | Not documented | 14 |
| AluSx3_dup11239 | Multi | No | — | — | SINE | Alu | chr7: 32973197-32973492 | − | 48 | 296 | AVL9 | Proximal | 0 | Not documented | 15 |
| AluSc_dup13544 | Multi/ | No | — | — | SINE | Alu | chr7: 33045699-33046025 | − | 40 | 327 | FKBP9 | Proximal | 0 | Not documented | 16 |
| | Uniquely | | | | | | | | | | | | | | |
| AluSx_dup50903 | Multi/ | No | — | — | SINE | Alu | chr7: 35002525-35002830 | + | 37 | 306 | DPY19L1 | Proximal | 0 | Not documented | 17 |
| | Uniquely | | | | | | | | | | | | | | |
| AluSz_dup35385 | Multi | No | — | — | SINE | Alu | chr7: 35672679-35672978 | − | 41 | 300 | HERPUD2 | Proximal | 0 | Not documented | 18 |
| LTR17_dup386 | Uniquely | No | — | — | LTR | ERV1 | chr7: 35735875-35736627 | + | 35 | 753 | HERPUD2 | Proximal | 695 | Not documented | 19 |
| Alulb_dup53125 | Uniquely | No | — | — | SINE | Alu | chr7: 35737511-35737817 | + | 74 | 307 | HERPUD2 | Distal | 2331 | Not documented | 20 |
| Zaphod3_dup587 | Multi/ | No | — | — | DNA | DNA | chr7: 37990351-37990499 | − | 81 | 149 | SFRP4 | Proximal | 0 | Not documented | 21 |
| | Uniquely | | | | | | | | | | | | | | |
| L2b_dup40693 | Multi/ | No | — | — | LINE | L2 | chr7: 37990853-37991200 | + | 140 | 348 | SFRP4 | Proximal | 0 | Not documented | 22 |
| | Uniquely | | | | | | | | | | | | | | |
| MIRc_dup43598 | Multi/ | No | — | — | SINE | MIR | chr7: 38218009-38218105 | − | 150 | 97 | STARD3NL | Proximal | 0 | Not documented | 23 |
| | Uniquely | | | | | | | | | | | | | | |
| L2a_dup74642 | Multi | No | — | — | LINE | L2 | chr7: 38765171-38765219 | − | 110 | 49 | VPS41 | Proximal | 0 | Not documented | 24 |
| L1MC4_dup12311 | Uniquely | No | — | — | LINE | Other L1 | chr7: 42941589-42942419 | + | 130 | 831 | C7orf25 | Distal | 5907 | Not documented | 4225 |
| MIR_dup74618 | Multi | No | — | — | SINE | MIR | chr7: 42975208-42975411 | − | 140 | 204 | MRPL32 | Proximal | 0 | Not documented | 26 |
| MLT1K_dup7816 | Multi/ | No | — | — | LTR | ERVL- | chr7: 43648624-43648787 | − | 120 | 164 | STK17A | Proximal | 0 | Not documented | 27 |
| | Uniquely | | | | | MaLR | | | | | | | | | |
| MLT1K_dup7817 | Multi/ | No | — | — | LTR | ERVL- | chr7: 43649033-43649141 | − | 120 | 109 | STK17A | Proximal | 0 | Not documented | 28 |
| | Uniquely | | | | | MaLR | | | | | | | | | |
| L1ME2_dup5546 | Uniquely | No | — | — | LINE | Other L1 | chr7: 43667891-43669122 | − | 110 | 1232 | COA1 | Proximal | 0 | Not documented | 29 |
| L2c_dup61931 | Multi/ | No | — | — | LINE | L2 | chr7: 43915507-43915743 | − | 97 | 237 | URGCP-MRPS24 | Proximal | 0 | Not documented | 30 |
| | Uniquely | | | | | | | | | | | | | | |
| AluSz_dup35667 | Multi/ | No | — | — | SINE | Alu | chr7: 43993857-43994165 | − | 47 | 309 | UBE2D4 | Proximal | 0 | Not documented | 31 |
| | Uniquely | | | | | | | | | | | | | | |
| AluSc_dup13649 | Multi | No | — | — | SINE | Alu | chr7: 44024192-44024480 | + | 42 | 289 | SPDYE1 | Distal | 16009 | Not documented | 32 |
| AluSz6_dup17350 | Multi | No | — | — | SINE | Alu | chr7: 44042637-44042976 | + | 70 | 340 | SPDYE1 | Proximal | 0 | Not documented | 33 |
| AluSx_dup51314 | Multi | No | — | — | SINE | Alu | chr7: 44051412-44051632 | − | 45 | 221 | SPDYE1 | Proximal | 1692 | Not documented | 34 |
| HERV4_I-int_{—} | Multi | No | — | — | LTR | ERV1 | chr7: 44051756-44052058 | + | 150 | 303 | SPDYE1 | Distal | 2036 | Not documented | 35 |
| dup133 | | | | | | | | | | | | | | | |
| AluSq2_dup20541 | Multi | No | — | — | SINE | Alu | chr7: 44052472-44052766 | − | 65 | 295 | SPDYE1 | Distal | 2752 | Not documented | 36 |
| HUERS-P3-int_{—} | Multi | No | — | — | LTR | ERV1 | chr7: 44052818-44052941 | + | 160 | 124 | SPDYE1 | Distal | 3098 | Not documented | 37 |
| dup190 | | | | | | | | | | | | | | | |
| HERV4_I-int_{—} | Multi | No | — | — | LTR | ERV1 | chr7: 44052946-44053091 | + | 170 | 146 | SPDYE1 | Distal | 3226 | Not documented | 38 |
| dup134 | | | | | | | | | | | | | | | |
| MSTA_dup8369 | Multi | No | — | — | LTR | ERVL- | chr7: 44053113-44053496 | − | 60 | 384 | SPDYE1 | Distal | 3393 | Not documented | 39 |
| | | | | | | MaLR | | | | | | | | | |
| AluSq2_dup20552 | Multi/ | No | — | — | SINE | Alu | chr7: 44618225-44618489 | + | 40 | 265 | TMED4 | Proximal | 0 | Not documented | 40 |
| | Uniquely | | | | | | | | | | | | | | |
| L1MB3_dup6647 | Multi/ | No | — | — | LINE | Other L1 | chr7: 44619554-44619796 | + | 63 | 243 | TMED4 | Proximal | 0 | Not documented | 41 |
| | Uniquely | | | | | | | | | | | | | | |
| AluSx_dup51354 | Multi/ | No | — | — | SINE | Alu | chr7: 44619797-44620091 | + | 46 | 295 | TMED4 | Proximal | 0 | Not documented | 42 |
| | Uniquely | | | | | | | | | | | | | | |
| AluSp_dup18777 | Multi/ | No | — | — | SINE | Alu | chr7: 44620264-44620566 | + | 37 | 303 | TMED4 | Proximal | 0 | Not documented | 43 |
| | Uniquely | | | | | | | | | | | | | | |
| AluSc5_dup2689 | Uniquely | No | — | — | SINE | Alu | chr7: 44837877-44838121 | + | 33 | 245 | PPIA | Proximal | 0 | Not documented | 44 |
| AluJr_dup27868 | Multi | No | — | — | SINE | Alu | chr7: 44841402-44841648 | + | 77 | 247 | PPIA | Proximal | 0 | Not documented | 45 |
| AluSx_dup51388 | Multi | No | — | — | SINE | Alu | chr7: 44841794-44842043 | − | 58 | 250 | PPIA | Proximal | 0 | Not documented | 46 |
| Charlie22a_dup501 | Multi | No | — | — | DNA | DNA | chr7: 44866402-44866535 | − | 100 | 134 | H2AFV | Proximal | 0 | Not documented | 47 |
| MIRb_dup94097 | Multi/ | No | — | — | SINE | MIR | chr7: 44868000-44868200 | − | 130 | 201 | H2AFV | Proximal | 0 | Not documented | 48 |
| | Uniquely | | | | | | | | | | | | | | |
| MLT1D_dup8208 | Multi/ | No | — | — | LTR | ERVL- | chr7: 44868296-44868740 | − | 96 | 445 | H2AFV | Proximal | 0 | Not documented | 49 |
| | Uniquely | | | | | MaLR | | | | | | | | | |
| AluSq2_dup20573 | Multi | No | — | — | SINE | Alu | chr7: 44868918-44869219 | + | 54 | 302 | H2AFV | Proximal | 0 | Not documented | 4250 |
| L1MC5_dup8505 | Uniquely | No | — | — | LINE | Other L1 | chr7: 44871527-44872120 | + | 110 | 594 | H2AFV | Proximal | 0 | Not documented | 51 |
| AluSz_dup35735 | Multi | No | — | — | SINE | Alu | chr7: 44917120-44917423 | + | 42 | 304 | PURB | Proximal | 0 | Not documented | 52 |
| AluSx1_dup39498 | Multi/ | No | — | — | SINE | Alu | chr7: 45759828-45760140 | + | 40 | 313 | ADCY1 | Proximal | 0 | Not documented | 53 |
| | Uniquely | | | | | | | | | | | | | | |
| MER9a2_dup130 | Multi | No | — | — | LTR | ERVK | chr7: 45765858-45766366 | − | 29 | 509 | ADCY1 | Distal | 3144 | Not documented | 54 |
| HERVK9-int_dup302 | Multi | No | — | — | LTR | ERVK | chr7: 45766367-45766484 | − | 20 | 118 | ADCY1 | Distal | 3653 | Not documented | 55 |
| LTR84b_dup616 | Multi | No | — | — | LTF | ERVL | chr7: 45796515-45796598 | − | 110 | 84 | ADCY1 | Distal | 33801 | Not documented | 56 |
| HERVK-int_dup102 | Multi | No | — | — | LTR | ERVK | chr7: 4623025-4630560 | − | 2.7 | 7536 | FOXK1 | Distal | 52829 | EVE annotated | 57 |
| L1PB4_dup2967 | Uniquely | No | — | — | LINE | L1PA\|B\|x | chr7: 467453-467998 | + | 96 | 546 | PDGFA | Distal | 68898 | Not documented | 58 |
| AluSc_dup13707 | Multi | No | — | — | SINE | Alu | chr7: 48003226-48003532 | + | 35 | 307 | HUS1 | Proximal | 0 | Not documented | 59 |
| AluJr_dup27951 | Multi | No | — | — | SINE | Alu | chr7: 48003698-48003995 | + | 73 | 298 | HUS1 | Proximal | 0 | Not documented | 60 |
| AluY_dup44788 | Multi | No | — | — | SINE | Alu | chr7: 4807753-4808062 | − | 3.2 | 310 | FOXK1 | Proximal | 0 | Not documented | 61 |
| AluSx_dup49618 | Multi | No | — | — | SINE | Alu | chr7: 4809884-4810172 | + | 41 | 289 | FOXK1 | Proximal | 0 | Not documented | 62 |
| L1M4_dup6481 | Multi | No | — | — | LINE | Other L1 | chr7: 48672234-48672852 | + | 95 | 619 | ABCA13 | Proximal | 0 | Not documented | 63 |
| L2a_dup72814 | Uniquely | No | — | — | LINE | L2 | chr7: 5037352-5037415 | − | 100 | 64 | RBAK-RBAKDN | Proximal | 0 | Not documented | 64 |
| L1MC4a_dup11599 | Uniquely | No | — | — | LINE | Other L1 | chr7: 50678880-50679077 | − | 120 | 198 | GRB10 | Proximal | 0 | Not documented | 65 |
| L1PA13_dup3803 | Uniquely | No | — | — | LINE | L1PA\|B\|x | chr7: 51261649-51264239 | − | 46 | 2591 | COBL | Proximal | 0 | EVE annotated | 66 |
| L1PA13_dup3804 | Uniquely | No | — | — | LINE | L1PA\|B\|x | chr7: 51264263-51266228 | − | 47 | 1966 | COBL | Proximal | 0 | Not documented | 67 |
| SVA_D_dup569 | Multi | No | — | — | Other | SVA | chr7: 51275135-51276762 | + | 13 | 1628 | COBL | Proximal | 0 | Not documented | 68 |
| MIRb_dup94508 | Uniquely | No | — | — | SINE | MIR | chr7: 51331597-51331800 | − | 150 | 204 | COBL | Proximal | 0 | Not documented | 69 |
| AluSx_dup51652 | Uniquely | No | — | — | SINE | Alu | chr7: 51332086-51332392 | + | 47 | 307 | COBL | Proximal | 0 | Not documented | 70 |
| L1PA16_dup6113 | Uniquely | No | — | — | LINE | L1PA\|B\|x | chr7: 51352941-51354960 | + | 83 | 2020 | COBL | Proximal | 0 | Not documented | 71 |
| L1MDa_dup2652 | Multi | No | — | — | LINE | Other L1 | chr7: 537461-537633 | + | 140 | 173 | PDGFA | Proximal | 0 | Not documented | 72 |
| PABL_A-int_dup166 | Uniquely | No | — | — | LTR | ERV1 | chr7: 54458202-54461001 | − | 43 | 2800 | VSTM2A | Distal | 149018 | Not documented | 73 |
| L1MA10_dup2033 | Uniquely | No | — | — | LINE | Other L1 | chr7: 54479356-54482223 | + | 94 | 2868 | VSTM2A | Distal | 127796 | Not documented | 74 |
| L1MEc_dup8263 | Uniquely | No | — | — | LINE | Other L1 | chr7: 54505487-54506550 | + | 110 | 1064 | VSTM2A | Distal | 103469 | Not documented | 4275 |
| L1MD1_dup2868 | Uniquely | No | — | — | LINE | Other L1 | chr7: 54506966-54509164 | − | 94 | 2199 | VSTM2A | Distal | 100855 | Not documented | 76 |
| AluSx1_dup39720 | Multi/ | No | — | — | SINE | Alu | chr7: 55091061-55091362 | + | 48 | 302 | EGFR | Proximal | 0 | Not documented | 77 |
| | Uniquely | | | | | | | | | | | | | | |
| L2_dup25010 | Uniquely | No | — | — | LINE | L2 | chr7: 55092817-55093321 | − | 140 | 505 | EGFR | Proximal | 0 | Not documented | 78 |
| MER5B_dup10566 | Multi/ | No | — | — | DNA | DNA | chr7: 55093702-55093852 | + | 140 | 151 | EGFR | Proximal | 0 | Not documented | 79 |
| | Uniquely | | | | | | | | | | | | | | |
| LTR19-int_dup53 | Multi | No | — | — | LTR | ERV1 | chr7: 55094042-55094099 | + | 31 | 58 | EGFR | Proximal | 0 | Not documented | 80 |
| LTR19A_dup143 | Multi/ | No | — | — | LTR | ERV1 | chr7: 55094103-55094559 | + | 75 | 457 | EGFR | Proximal | 0 | Not documented | 81 |
| | Uniquely | | | | | | | | | | | | | | |
| MER5A_dup15167 | Uniquely | No | — | — | DNA | DNA | chr7: 55094594-55094768 | + | 110 | 175 | EGFR | Proximal | 0 | Not documented | 82 |
| AluSx1_dup39721 | Uniquely | No | — | — | SINE | Alu | chr7: 55101912-55102138 | − | 54 | 227 | EGFR | Proximal | 0 | Not documented | 83 |
| AluSz6_dup17452 | Multi/ | No | — | — | SINE | Alu | chr7: 55103075-55103406 | + | 63 | 332 | EGFR | Proximal | 0 | Not documented | 84 |
| | Uniquely | | | | | | | | | | | | | | |
| MIRb_dup94650 | Uniquely | No | — | — | SINE | MIR | chr7: 55113273-55113459 | + | 130 | 187 | EGFR | Proximal | 0 | Not documented | 85 |
| L1MB3_dup6727 | Uniquely | No | — | — | LINE | Other L1 | chr7: 55117760-55118254 | − | 80 | 495 | EGFR | Proximal | 0 | Not documented | 86 |
| MSTD_dup3518 | Multi/ | No | — | — | LTR | ERVL- | chr7: 55118954-55119348 | + | 74 | 395 | EGFR | Proximal | 0 | Not documented | 87 |
| | Uniquely | | | | | MaLR | | | | | | | | | |
| L2c_dup62186 | Multi | No | — | — | LINE | L2 | chr7: 55119431-55119617 | + | 110 | 187 | EGFR | Proximal | 0 | Not documented | 88 |
| L3_dup19823 | Uniquely | No | — | — | LINE | CR1 | chr7: 55124446-55124999 | + | 160 | 554 | EGFR | Proximal | 0 | Not documented | 89 |
| L1PA13_dup3814 | Multi | No | — | — | LINE | L1PA\|B\|x | chr7: 55125729-55125806 | + | 48 | 78 | EGFR | Proximal | 0 | Not documented | 90 |
| L1PA13_dup3815 | Multi/ | No | — | — | LINE | L1PA\|B\|x | chr7: 55126103-55126544 | + | 48 | 442 | EGFR | Proximal | 0 | Not documented | 91 |
| | Uniquely | | | | | | | | | | | | | | |
| L1MC2_dup2596 | Uniquely | No | — | — | LINE | Other L1 | chr7: 55126987-55127368 | − | 100 | 382 | EGFR | Proximal | 0 | Not documented | 92 |
| MIRb_dup94653 | Uniquely | No | — | — | SINE | MIR | chr7: 55153739-55153939 | − | 130 | 201 | EGFR | Proximal | 0 | Not documented | 93 |
| MER5A_dup15169 | Multi/ | No | — | — | DNA | DNA | chr7: 55164808-55164907 | + | 76 | 100 | EGFR | Proximal | 0 | Not documented | 94 |
| | Uniquely | | | | | | | | | | | | | | |
| L1M5_dup27328 | Multi/ | No | — | — | LINE | Other L1 | chr7: 55165080-55165167 | + | 100 | 88 | EGFR | Proximal | 0 | Not documented | 95 |
| | Uniquely | | | | | | | | | | | | | | |
| Arthur1B_dup1100 | Multi/ | No | — | — | DNA | DNA | chr7: 55165205-55165290 | + | 94 | 86 | EGFR | Proximal | 0 | Not documented | 96 |
| | Uniquely | | | | | | | | | | | | | | |
| L3_dup19827 | Multi/ | No | — | — | LINE | CR1 | chr7: 55272886-55272949 | + | 120 | 64 | EGFR | Proximal | 0 | Not documented | 97 |
| | Uniquely | | | | | | | | | | | | | | |
| L2c_dup62191 | Multi/ | No | — | — | LINE | L2 | chr7: 55275066-55275362 | − | 140 | 297 | EGFR | Proximal | 0 | Not documented | 98 |
| | Uniquely | | | | | | | | | | | | | | |
| MER5B_dup10568 | Multi/ | No | — | — | DNA | DNA | chr7: 55276536-55276648 | − | 140 | 113 | EGFR | Proximal | 0 | Not documented | 99 |
| | Uniquely | | | | | | | | | | | | | | |
| MER5B_dup10569 | Multi/ | No | — | — | DNA | DNA | chr7: 55276793-55276903 | + | 120 | 111 | EGFR | Proximal | 0 | Not documented | 4300 |
| | Uniquely | | | | | | | | | | | | | | |
| MER5B_dup10570 | Multi/ | No | — | — | DNA | DNA | chr7: 55278068-55278232 | + | 88 | 165 | EGFR | Proximal | 0 | Not documented | 1 |
| | Uniquely | | | | | | | | | | | | | | |
| MER34_dup639 | Uniquely | No | — | — | LTR | ERV1 | chr7: 55434560-55434841 | − | 110 | 282 | LANCL2 | Proximal | 0 | Not documented | 2 |
| AluSz6_dup17457 | Uniquely | No | — | — | SINE | Alu | chr7: 55435136-55435422 | + | 47 | 287 | LANCL2 | Proximal | 0 | Not documented | 3 |
| AluSg_dup15920 | Multi | No | — | — | SINE | Alu | chr7: 55464873-55465179 | + | 41 | 307 | LANCL2 | Proximal | 0 | Not documented | 4 |
| Tigger1_dup5553 | Multi | No | — | — | DNA | DNA | chr7: 55519499-55521571 | − | 56 | 2073 | VOPP1 | Proximal | 0 | Not documented | 5 |
| L1MA4A_dup2692 | Multi | No | — | — | LINE | Other L1 | chr7: 55524647-55528304 | − | 67 | 3658 | VOPP1 | Proximal | 0 | Not documented | 6 |
| L1MA4A_dup2695 | Multi | No | — | — | LINE | Other L1 | chr7: 55529409-55530348 | − | 87 | 940 | VOPP1 | Proximal | 0 | Not documented | 7 |
| AluYa5_dup1725 | Multi | No | — | — | SINE | Alu | chr7: 55530349-55530610 | − | 3.6 | 262 | VOPP1 | Proximal | 0 | Not documented | 8 |
| L1MA4A_dup2696 | Multi | No | — | — | LINE | Other L1 | chr7: 55530611-55532901 | − | 89 | 2291 | VOPP1 | Proximal | 0 | Not documented | 9 |
| AluSc_dup13761 | Multi | No | — | — | SINE | Alu | chr7: 55701030-55701336 | − | 46 | 307 | VOPP1 | Distal | 60350 | Not documented | 10 |
| AluSc_dup13763 | Multi | No | — | — | SINE | Alu | chr7: 55749282-55749606 | + | 43 | 325 | VOPP1 | Distal | 108602 | Not documented | 11 |
| AluSc8_dup8405 | Multi | No | — | — | SINE | Alu | chr7: 55946146-55946436 | + | 45 | 291 | MRPS17 | Distal | 8535 | Not documented | 12 |
| AluY_dup46706 | Multi | No | — | — | SINE | Alu | chr7: 56023134-56023433 | − | 30 | 300 | GBAS | Proximal | 0 | Not documented | 13 |
| AluJo_dup26051 | Multi | No | — | — | SINE | Alu | chr7: 56023459-56023751 | − | 85 | 293 | GBAS | Proximal | 0 | Not documented | 14 |
| AluSp_dup18927 | Multi | No | — | — | SINE | Alu | chr7: 56023821-56024103 | − | 46 | 283 | GBAS | Proximal | 0 | Not documented | 15 |
| L3_dup19835 | Multi/ | No | — | — | LINE | CR1 | chr7: 56046306-56046616 | − | 130 | 311 | GBAS | Proximal | 0 | Not documented | 16 |
| | Uniquely | | | | | | | | | | | | | | |
| AluSx1_dup39785 | Multi | No | — | — | SINE | Alu | chr7: 56052128-56052428 | + | 48 | 301 | GBAS | Proximal | 0 | Not documented | 17 |
| AluY_dup46712 | Multi/ | No | — | — | SINE | Alu | chr7: 56078941-56079235 | + | 28 | 295 | PSPH | Proximal | 0 | Not documented | 18 |
| | Uniquely | | | | | | | | | | | | | | |
| L1M5_dup27361 | Multi | No | — | — | LINE | Other L1 | chr7: 56131064-56131191 | − | 100 | 128 | CCT6A | Proximal | 0 | Not documented | 19 |
| AluSx1_dup39797 | Multi/ | No | — | — | SINE | Alu | chr7: 56132285-56132603 | + | 30 | 319 | SUMF2 | Proximal | 0 | Not documented | 20 |
| | Uniquely | | | | | | | | | | | | | | |
| AluJr_dup28073 | Multi/ | No | — | — | SINE | Alu | chr7: 56146596-56146866 | − | 79 | 271 | SUMF2 | Proximal | 0 | Not documented | 21 |
| | Uniquely | | | | | | | | | | | | | | |
| AluJr_dup28074 | Multi/ | No | — | — | SINE | Alu | chr7: 56147951-56148254 | − | 55 | 304 | SUMF2 | Proximal | 0 | Not documented | 22 |
| | Uniquely | | | | | | | | | | | | | | |
| LTR89_dup488 | Multi/ | No | — | — | LTR | ERVL | chr7: 56148299-56148402 | + | 150 | 104 | SUMF2 | Proximal | 0 | Not documented | 23 |
| | Uniquely | | | | | | | | | | | | | | |
| AluJb_dup53916 | Multi | No | — | — | SINE | Alu | chr7: 56156054-56156233 | + | 94 | 180 | PHKG1 | Proximal | 0 | Not documented | 24 |
| AluJr_dup28076 | Multi | No | — | — | SINE | Alu | chr7: 56156233-56156349 | − | 50 | 117 | PHKG1 | Proximal | 0 | Not documented | 4325 |
| AluJo_dup26079 | Multi | No | — | — | SINE | Alu | chr7: 56168912-56169067 | − | 73 | 156 | CHCHD2 | Proximal | 196 | Not documented | 26 |
| AluJo_dup26080 | Multi | No | — | — | SINE | Alu | chr7: 56169668-56169787 | − | 45 | 120 | CHCHD2 | Proximal | 0 | Not documented | 27 |
| AluSx1_dup39805 | Multi/ | No | — | — | SINE | Alu | chr7: 56170039-56170360 | − | 44 | 322 | CHCHD2 | Proximal | 0 | Not documented | 28 |
| | Uniquely | | | | | | | | | | | | | | |
| AluSp_dup18263 | Multi | No | — | — | SINE | Alu | chr7: 5660803-5661081 | − | 49 | 279 | RNF216 | Proximal | 0 | Not documented | 29 |
| AluSz_dup34668 | Multi | No | — | — | SINE | Alu | chr7: 5940834-5941134 | + | 66 | 301 | CCZ1 | Proximal | 0 | Not documented | 30 |
| L1M3a_dup215 | Multi | No | — | — | LINE | Other L1 | chr7: 63372711-63373221 | − | 140 | 511 | ZNF727 | Distal | 132601 | Not documented | 31 |
| L1PREC2_dup3395 | Uniquely | No | — | — | LINE | L1PA\|B\|x | chr7: 63811180-63813818 | − | 110 | 2639 | ZNF736 | Proximal | 0 | Not documented | 32 |
| AluSq_dup8207 | Uniquely | No | — | — | SINE | Alu | chr7: 64170222-64170525 | + | 67 | 304 | ZNF107 | Proximal | 0 | Not documented | 33 |
| L1MA3_dup4009 | Multi | No | — | — | LINE | Other L1 | chr7: 64435163-64435363 | + | 69 | 201 | ZNF117 | Proximal | 0 | Not documented | 34 |
| MER57E3_dup61 | Multi | No | — | — | LTR | ERV1 | chr7: 64466074-64466559 | + | 61 | 486 | ERV3-1 | Proximal | 0 | Not documented | 35 |
| L1ME4a_dup18785 | Multi | No | — | — | LINE | Other L1 | chr7: 64534498-64534767 | − | 120 | 270 | ERV3-1 | Distal | 67378 | Not documented | 36 |
| L1ME4a_dup18794 | Multi | No | — | — | LINE | Other L1 | chr7: 65228616-65228885 | − | 120 | 270 | VKORC1L1 | Distal | 109370 | Not documented | 37 |
| AluSp_dup19152 | Multi | No | — | — | SINE | Alu | chr7: 65618144-65618438 | + | 54 | 295 | CRCP | Proximal | 0 | Not documented | 38 |
| AluSz6_dup17619 | Multi | No | — | — | SINE | Alu | chr7: 65619158-65619381 | − | 55 | 224 | CRCP | Proximal | 0 | Not documented | 39 |
| MLT2C2_dup588 | Multi | No | — | — | LTR | ERVL | chr7: 65703499-65703779 | − | 75 | 281 | TPST1 | Proximal | 0 | Not documented | 40 |
| L2c_dup62360 | Multi | No | — | — | LINE | L2 | chr7: 65704032-65704142 | + | 150 | 111 | TPST1 | Proximal | 0 | Not documented | 41 |
| FLAM_C_dup8427 | Multi | No | — | — | SINE | Alu | chr7: 65704143-65704267 | − | 80 | 125 | TPST1 | Proximal | 0 | Not documented | 42 |
| L2c_dup62361 | Multi | No | — | — | LINE | L2 | chr7: 65704268-65704411 | + | 150 | 144 | TPST1 | Proximal | 0 | Not documented | 43 |
| AluSx_dup52434 | Multi | Nc | — | — | SINE | Alu | chr7: 65708431-65708734 | − | 73 | 304 | TPST1 | Proximal | 0 | Not documented | 44 |
| AluY_dup47109 | Multi | No | — | — | SINE | Alu | chr7: 65717345-65717619 | − | 21 | 275 | TPST1 | Proximal | 0 | Not documented | 45 |
| L1MEf_dup6099 | Uniquely | No | — | — | LINE | Other L1 | chr7: 65762962-65763785 | − | 100 | 824 | TPST1 | Proximal | 0 | Not documented | 46 |
| L1MEf_dup6100 | Multi | No | — | — | LINE | Other L1 | chr7: 65764187-65764539 | − | 94 | 353 | TPST1 | Proximal | 0 | Not documented | 47 |
| L1MEf_dup6101 | Multi | No | — | — | LINE | Other L1 | chr7: 65764578-65764805 | − | 120 | 228 | TPST1 | Proximal | 0 | Not documented | 48 |
| Tigger1_dup5594 | Multi | No | — | — | DNA | DNA | chr7: 65764856-65765197 | − | 65 | 342 | TPST1 | Proximal | 0 | Not documented | 49 |
| Tigger1_dup5595 | Multi/ | No | — | — | DNA | DNA | chr7: 65765201-65765650 | − | 50 | 450 | TPST1 | Proximal | 0 | Not documented | 4350 |
| | Uniquely | | | | | | | | | | | | | | |
| X7C_LINE_dup269 | Uniquely | No | — | — | LINE | CR1 | chr7: 65776844-65777047 | − | 120 | 204 | TPST1 | Proximal | 0 | Not documented | 51 |
| L1M6_dup3128 | Multi/ | No | — | — | LINE | Other L1 | chr7: 65777700-65778121 | + | 130 | 422 | TPST1 | Proximal | 0 | Not documented | 52 |
| | Uniquely | | | | | | | | | | | | | | |
| AluSx1_dup40298 | Multi | No | — | — | SINE | Alu | chr7: 65782864-65783171 | + | 53 | 308 | TPST1 | Proximal | 0 | Not documented | 53 |
| L1MEe_dup5296 | Multi/ | No | — | — | LINE | Other L1 | chr7: 65783172-65783458 | − | 120 | 287 | TPST1 | Proximal | 0 | Not documented | 54 |
| | Uniquely | | | | | | | | | | | | | | |
| L1M1_dup3889 | Multi/ | No | — | — | LINE | Other L1 | chr7: 65802596-65804639 | + | 70 | 2044 | TPST1 | Proximal | 0 | Not documented | 55 |
| | Uniquely | | | | | | | | | | | | | | |
| L1MCb_dup967 | Multi | No | — | — | LINE | Other L1 | chr7: 65808558-65809101 | + | 84 | 544 | TPST1 | Proximal | 0 | Not documented | 56 |
| AluSx_dup52445 | Multi | No | — | — | SINE | Alu | chr7: 65819313-65819529 | + | 57 | 217 | TPST1 | Proximal | 0 | Not documented | 57 |
| AluSq10_dup837 | Uniquely | No | — | — | SINE | Alu | chr7: 66025409-66025705 | + | 41 | 297 | KCTD7 | Distal | 68164 | Not documented | 58 |
| AluJr_dup28488 | Multi/ | No | — | — | SINE | Alu | chr7: 66204968-66205223 | − | 64 | 256 | RABGEF1 | Proximal | 0 | Not documented | 59 |
| | Uniquely | | | | | | | | | | | | | | |
| AluJr_dup28517 | Multi/ | No | — | — | SINE | Alu | chr7: 66421662-66421961 | − | 59 | 300 | TMEM248 | Proximal | 0 | Not documented | 60 |
| | Uniquely | | | | | | | | | | | | | | |
| FRAM_dup3284 | Multi/ | No | — | — | SINE | Alu | chr7: 66745248-66745406 | + | 83 | 159 | TYW1 | Distal | 40748 | Not documented | 61 |
| | Uniquely | | | | | | | | | | | | | | |
| AluSz_dup36397 | Multi | No | — | — | SINE | Alu | chr7: 66745407-66745708 | + | 71 | 302 | TYW1 | Distal | 40907 | Not documented | 62 |
| AluSx1_dup40432 | Multi | No | — | — | SINE | Alu | chr7: 66745788-66746111 | + | 52 | 324 | TYW1 | Distal | 41288 | Not documented | 63 |
| MER21C_dup2285 | Multi | No | — | — | LTR | ERVL | chr7: 66755448-66755662 | − | 120 | 215 | TYW1 | Distal | 50948 | Not documented | 64 |
| MER84-int_dup56 | Multi | No | — | — | LTR | ERV1 | chr7: 66755928-66756311 | + | 140 | 384 | TYW1 | Distal | 51428 | Not documented | 65 |
| AluSq2_dup21049 | Multi | No | — | — | SINE | Alu | chr7: 66756725-66757024 | − | 68 | 300 | TYW1 | Distal | 52225 | Not documented | 66 |
| MSTA_dup8478 | Multi | No | — | — | LTR | ERVL- | chr7: 66757370-66757754 | − | 58 | 385 | TYW1 | Distal | 52870 | Not documented | 67 |
| | | | | | | MaLR | | | | | | | | | |
| AluSz_dup34796 | Multi | No | — | — | SINE | Alu | chr7: 6863144-6863449 | − | 65 | 306 | CCZ1B | Proximal | 0 | Not documented | 68 |
| MLT1L_dup5478 | Uniquely | No | — | — | LTR | ERVL- | chr7: 69494989-69495190 | − | 130 | 202 | AUTS2 | Proximal | 0 | Not documented | 69 |
| | | | | | | MaLR | | | | | | | | | |
| AluSz6_dup17847 | Multi | No | — | — | SINE | Alu | chr7: 72338782-72339114 | − | 75 | 333 | POM121 | Distal | 10823 | Not documented | 70 |
| AluSz_dup36853 | Multi | No | — | — | SINE | Alu | chr7: 72340196-72340495 | − | 73 | 300 | POM121 | Distal | 9442 | Not documented | 71 |
| AluJo_dup26804 | Multi | No | — | — | SINE | Alu | chr7: 72340500-72340655 | − | 79 | 156 | POM121 | Distal | 9282 | Not documented | 72 |
| LTR5B_dup146 | Multi | No | — | — | LTR | ERVK | chr7: 72421753-72422735 | + | 31 | 983 | POM121 | Proximal | 0 | Not documented | 73 |
| AluSx_dup53182 | Multi | No | — | — | SINE | Alu | chr7: 72422736-72422883 | + | 44 | 148 | POM121 | Proximal | 758 | Not documented | 74 |
| AluJb_dup55032 | Multi | No | — | — | SINE | Alu | chr7: 72463820-72464164 | − | 76 | 345 | AC005488.1 | Proximal | 0 | Not documented | 4375 |
| L1ME3B_dup3494 | Multi | No | — | — | LINE | Other L1 | chr7: 72465108-72465468 | − | 120 | 361 | AC005488.1 | Proximal | 113 | Not documented | 76 |
| FLAM_C_dup8515 | Multi | No | — | — | SINE | Alu | chr7: 72469014-72469148 | − | 57 | 135 | AC005488.1 | Distal | 4019 | Not documented | 77 |
| AluJo_dup26821 | Multi | No | — | — | SINE | Alu | chr7: 72477090-72477221 | − | 69 | 132 | AC005488.1 | Distal | 12095 | Not documented | 78 |
| AluSx1_dup40952 | Multi | No | — | — | SINE | Alu | chr7: 72477513-72477812 | + | 50 | 300 | AC005488.1 | Distal | 12518 | Not documented | 79 |
| MSTA_dup8562 | Multi | No | — | — | LTR | ERVL- | chr7: 72486639-72487025 | + | 70 | 387 | AC005488.1 | Distal | 21644 | Not documented | 80 |
| | | | | | | MaLR | | | | | | | | | |
| AluJo_dup26823 | Multi | No | — | — | SINE | Alu | chr7: 72496606-72496945 | − | 87 | 340 | AC005488.1 | Distal | 31611 | Not documented | 81 |
| MSTA_dup8563 | Multi | No | — | — | LTR | ERVL- | chr7: 72514701-72515087 | + | 70 | 387 | AC005488.1 | Distal | 49706 | Not documented | 82 |
| | | | | | | MaLR | | | | | | | | | |
| AluJo_dup26826 | Multi | No | — | — | SINE | Alu | chr7: 72524668-72525007 | − | 85 | 340 | AC005488.1 | Distal | 59673 | Not documented | 83 |
| MER58A_dup5596 | Multi | No | — | — | DNA | DNA | chr7: 72614869-72615059 | + | 110 | 191 | NSUN5 | Distal | 101456 | Not documented | 84 |
| AluSx1_dup40981 | Multi | No | — | — | SINE | Alu | chr7: 72615060-72615352 | + | 51 | 293 | NSUN5 | Distal | 101163 | Not documented | 85 |
| AluSx_dup53235 | Multi | No | — | — | SINE | Alu | chr7: 72656923-72657216 | + | 43 | 294 | NSUN5 | Distal | 59299 | Not documented | 86 |
| Charlie8_dup1228 | Multi | No | — | — | DNA | DNA | chr7: 72657217-72659145 | − | 92 | 1929 | NSUN5 | Distal | 57370 | Not documented | 87 |
| L1ME3B_dup3497 | Multi | No | — | — | LINE | Other L1 | chr7: 72700053-72700413 | − | 110 | 361 | NSUN5 | Distal | 16102 | Not documented | 88 |
| L2c_dup62703 | Multi | No | — | — | LINE | L2 | chr7: 72983945-72984065 | + | 100 | 121 | TBL2 | Proximal | 0 | Not documented | 89 |
| MIRb_dup95410 | Multi | No | — | — | SINE | MIR | chr7: 72984274-72984365 | − | 120 | 92 | TBL2 | Proximal | 0 | Not documented | 90 |
| L2a_dup75891 | Uniquely | No | — | — | LINE | L2 | chr7: 73109879-73110171 | + | 130 | 293 | WBSCR22 | Proximal | 0 | Not documented | 91 |
| L2a_dup75892 | Uniquely | No | — | — | LINE | L2 | chr7: 73110213-73110381 | + | 160 | 169 | WBSCR22 | Proximal | 0 | Not documented | 92 |
| FLAM_C_dup8545 | Multi | No | — | — | SINE | Alu | chr7: 73112344-73112482 | + | 75 | 139 | WBSCR22 | Proximal | 0 | Not documented | 93 |
| AluSz_dup36974 | Multi | No | — | — | SINE | Alu | chr7: 73150569-73150869 | − | 42 | 301 | ABHD11 | Proximal | 0 | Not documented | 94 |
| AluSx1_dup41144 | Multi/ | No | — | — | SINE | Alu | chr7: 73646009-73646309 | − | 48 | 301 | RFC2 | Proximal | 0 | Not documented | 95 |
| | Uniquely | | | | | | | | | | | | | | |
| MER58A_dup5601 | Multi | No | — | — | DNA | DNA | chr7: 74168558-74168748 | + | 110 | 191 | GTF21 | Proximal | 0 | Not documented | 96 |
| AluSx_dup53569 | Multi | No | — | — | SINE | Alu | chr7: 74210623-74210916 | + | 45 | 294 | GTF2IRD2 | Proximal | 0 | Not documented | 97 |
| Charlie8_dup1236 | Multi | No | — | — | DNA | DNA | chr7: 74210917-74212845 | − | 91 | 1929 | GTF2IRD2 | Proximal | 0 | Not documented | 98 |
| L1MB7_dup8882 | Multi | No | — | — | LINE | Other L1 | chr7: 74266568-74267052 | + | 75 | 485 | GTF2IRD2 | Proximal | 0 | Not documented | 99 |
| AluJb_dup55336 | Multi | No | — | — | SINE | Alu | chr7: 74294086-74294437 | − | 76 | 352 | GTF2IRD2 | Distal | 26240 | Not documented | 4400 |
| L1ME3B_dup3503 | Multi | No | — | — | LINE | Other L1 | chr7: 74295379-74295739 | − | 110 | 361 | GTF2IRD2 | Distal | 27533 | Not documented | 1 |
| FLAM_C_dup8587 | Multi | No | — | — | SINE | Alu | chr7: 74299270-74299404 | − | 50 | 135 | GTF2IRD2 | Distal | 31424 | Not documented | 2 |
| AluJo_dup27071 | Multi | No | — | — | SINE | Alu | chr7: 74307359-74307490 | − | 65 | 132 | GTF2IRD2 | Distal | 39513 | Not documented | 3 |
| AluSx1_dup41299 | Multi | No | — | — | SINE | Alu | chr7: 74307782-74308081 | + | 50 | 300 | GTF2IRD2 | Distal | 39936 | Not documented | 4 |
| MSTA_dup8568 | Multi | No | — | — | LTR | ERVL- | chr7: 74316785-74317171 | + | 74 | 387 | GTF2IRD2 | Distal | 48939 | Not documented | 5 |
| | | | | | | MaLR | | | | | | | | | |
| AluJo_dup27073 | Multi | No | — | — | SINE | Alu | chr7: 74327470-74327809 | − | 85 | 340 | GATSL1 | Distal | 51275 | Not documented | 6 |
| Charlie8_dup1239 | Multi | No | — | — | DNA | DNA | chr7: 74563259-74565187 | + | 91 | 1929 | GTF2IRD2B | Proximal | 0 | Not documented | 7 |
| AluSx_dup53671 | Multi | No | — | — | SINE | Alu | chr7: 74565188-74565482 | − | 45 | 295 | GTF2IRD2B | Proximal | 0 | Not documented | 8 |
| MER58A_dup5606 | Multi | No | — | — | DNA | DNA | chr7: 74607379-74607569 | − | 110 | 191 | GTF2IRD2B | Distal | 41757 | Not documented | 9 |
| AluJo_dup27122 | Multi | No | — | — | SINE | Alu | chr7: 74697616-74697955 | + | 85 | 340 | GATSL2 | Distal | 109545 | Not documented | 10 |
| MSTA_dup8577 | Multi | No | — | — | LTR | ERVL- | chr7: 74707535-74707921 | − | 70 | 387 | GATSL2 | Distal | 99579 | Not documented | 11 |
| | | | | | | MaLR | | | | | | | | | |
| AluJo_dup27146 | Multi | No | — | — | SINE | Alu | chr7: 74911880-74912219 | + | 87 | 340 | GATSL2 | Distal | 44372 | Not documented | 12 |
| MSTA_dup8583 | Multi | No | — | — | LTR | ERVL- | chr7: 74921800-74922186 | − | 70 | 387 | GATSL2 | Distal | 54292 | Not documented | 13 |
| | | | | | | MaLR | | | | | | | | | |
| AluJo_dup27149 | Multi | No | — | — | SINE | Alu | chr7: 74939900-74940239 | + | 85 | 340 | AC006014.1 | Distal | 59670 | Not documented | 14 |
| MSTA_dup8584 | Multi | No | — | — | LTR | ERVL- | chr7: 74949822-74950208 | − | 70 | 387 | AC006014.1 | Distal | 49701 | Not documented | 15 |
| | | | | | | MaLR | | | | | | | | | |
| AluJo_dup27152 | Multi | No | — | — | SINE | Alu | chr7: 74967923-74968255 | + | 86 | 333 | AC006014.1 | Distal | 31654 | Not documented | 16 |
| MSTA_dup8585 | Multi | No | — | — | LTR | ERVL- | chr7: 74977843-74978225 | − | 69 | 383 | AC006014.1 | Distal | 21684 | Not documented | 17 |
| | | | | | | MaLR | | | | | | | | | |
| AluSx1_dup41438 | Multi | No | — | — | SINE | Alu | chr7: 74987083-74987382 | − | 50 | 300 | AC006014.1 | Distal | 12527 | Not documented | 18 |
| AluJo_dup27154 | Multi | No | — | — | SINE | Alu | chr7: 74987674-74987805 | + | 69 | 132 | AC006014.1 | Distal | 12104 | Not documented | 19 |
| FLAM_C_dup8605 | Multi | No | — | — | SINE | Alu | chr7: 74995758-74995892 | + | 57 | 135 | AC006014.1 | Distal | 4017 | Not documented | 20 |
| L1ME3B_dup3506 | Multi | No | — | — | LINE | Other L1 | chr7: 74999436-74999785 | + | 110 | 350 | AC006014.1 | Proximal | 124 | Not documented | 21 |
| AluJb_dup55477 | Multi | No | — | — | SINE | Alu | chr7: 75000740-75001084 | + | 78 | 345 | AC006014.1 | Proximal | 0 | Not documented | 22 |
| AluSx_dup53763 | Multi | No | — | — | SINE | Alu | chr7: 75042021-75042165 | − | 43 | 145 | TRIM73 | Proximal | 1743 | Not documented | 23 |
| LTR5B_dup148 | Multi | No | — | — | LTR | ERVK | chr7: 75042166-75043148 | − | 32 | 983 | TRIM73 | Proximal | 1888 | Not documented | 24 |
| MIR_dup75875 | Multi | No | — | — | SINE | MIR | chr7: 75043835-75044030 | − | 130 | 196 | POM121C | Distal | 2037 | Not documented | 4425 |
| MIR3_dup38948 | Multi | No | — | — | SINE | MIR | chr7: 75045095-75045181 | + | 120 | 87 | POM121C | Proximal | 886 | Not documented | 16 |
| AluJo_dup27172 | Multi | No | — | — | SINE | Alu | chr7: 75124917-75125072 | + | 76 | 156 | POM121C | Distal | 9370 | Not documented | 17 |
| AluSz_dup37321 | Multi | No | — | — | SINE | Alu | chr7: 75125077-75125356 | + | 76 | 280 | POM121C | Distal | 9530 | Not documented | 18 |
| AluSx_dup53780 | Multi | No | — | — | SINE | Alu | chr7: 75135319-75135539 | − | 50 | 22 | POM121C | Distal | 19772 | Not documented | 19 |
| AluSq2_dup21419 | Multi | No | — | — | SINE | Alu | chr7: 75136379-75136678 | − | 67 | 300 | POM121C | Distal | 20832 | Not documented | 20 |
| MER84-int_dup66 | Multi | No | — | — | LTR | ERV1 | chr7: 75136877-75137003 | + | 160 | 127 | POM121C | Distal | 21330 | Not documented | 21 |
| MSTA_dup8588 | Multi | No | — | — | LTR | ERVL- | chr7: 75137024-75137407 | − | 57 | 384 | POM121C | Distal | 21477 | Not documented | 22 |
| | | | | | | MaLR | | | | | | | | | |
| MIRb_dup95580 | Multi/ | No | — | — | SINE | MIR | chr7: 75517732-75517874 | − | 120 | 143 | RHBDD2 | Proximal | 0 | Not documented | 23 |
| | Uniquely | | | | | | | | | | | | | | |
| AluJr_dup29729 | Multi | No | — | — | SINE | Alu | chr7: 75932585-75932670 | + | 21 | 86 | HSPB1 | Proximal | 0 | Not documented | 24 |
| AluSc_dup13304 | Multi | No | — | — | SINE | Alu | chr7: 7605410-7605713 | + | 39 | 304 | MIOS | Proximal | 791 | Not documented | 4425 |
| L2b_dup39791 | Multi/ | No | — | — | LINE | L2 | chr7: 7605823-7606208 | + | 120 | 386 | MIOS | Proximal | 296 | Not documented | 26 |
| | Uniquely | | | | | | | | | | | | | | |
| MSTA_dup8590 | Multi | No | — | — | LTR | ERVL- | chr7: 76156865-76157248 | + | 58 | 384 | UPK3B | Proximal | 0 | Not documented | 27 |
| | | | | | | MaLR | | | | | | | | | |
| AluSq2_dup21490 | Multi | No | — | — | SINE | Alu | chr7: 76157594-76157893 | + | 67 | 300 | UPK3B | Proximal | 0 | Not documented | 28 |
| AluSz6_dup18073 | Multi | No | — | — | SINE | Alu | chr7: 76167343-76167682 | − | 72 | 340 | UPK3B | Proximal | 0 | Not documented | 29 |
| AluSx1_dup41616 | Multi | No | — | — | SINE | Alu | chr7: 76168385-76168693 | − | 59 | 309 | UPK3B | Proximal | 0 | Not documented | 30 |
| AluSz_dup37465 | Multi | No | — | — | SINE | Alu | chr7: 76168762-76169061 | − | 71 | 300 | UPK3B | Proximal | 0 | Not documented | 31 |
| AluJo_dup27341 | Multi | No | — | — | SINE | Alu | chr7: 76169066-76169221 | − | 76 | 156 | UPK3B | Proximal | 0 | Not documented | 32 |
| AluSx1_dup41619 | Multi/ | No | — | — | SINE | Alu | chr7: 76183852-76184176 | − | 43 | 325 | UPK3B | Proximal | 0 | Not documented | 33 |
| | Uniquely | | | | | | | | | | | | | | |
| THE1C-int_dup852 | Uniquely | No | — | — | LTR | ERVL- | chr7: 76222376-76223565 | − | 54 | 1190 | UPK3B | Proximal | 0 | Not documented | 34 |
| | | | | | | MaLR | | | | | | | | | |
| L1PA15_dup3556 | Multi | No | — | — | LINE | L1PA\|B\|x | chr7: 76223935-76228295 | + | 66 | 4361 | UPK3B | Proximal | 0 | Not documented | 35 |
| AluJo_dup27366 | Multi | No | — | — | SINE | Alu | chr7: 76658004-76658331 | − | 90 | 328 | UPK3B | Distal | 9665 | Not documented | 36 |
| AluSz_dup37482 | Multi | No | — | — | SINE | Alu | chr7: 76687816-76688113 | − | 69 | 298 | UPK3B | Distal | 39477 | Not documented | 37 |
| AluSx_dup54109 | Multi | No | — | — | SINE | Alu | chr7: 77233527-77233830 | − | 48 | 304 | PTPN12 | Proximal | 0 | Not documented | 38 |
| AluSq2_dup21525 | Multi | No | — | — | SINE | Alu | chr7: 77235263-77235570 | − | 60 | 308 | PTPN12 | Proximal | 0 | Not documented | 39 |
| AluJb_dup55762 | Multi | No | — | — | SINE | Alu | chr7: 77235715-77235874 | − | 88 | 160 | PTPN12 | Proximal | 0 | Not documented | 4450 |
| Charlie7a_dup713 | Multi | No | — | — | DNA | DNA | chr7: 77313169-77313351 | − | 120 | 183 | RSBN1L | Dista | 12410 | Not documented | 51 |
| MIR_dup75994 | Uniquely | No | — | — | SINE | MIR | chr7: 77671805-77672037 | + | 130 | 233 | MAGI2 | Proximal | 0 | Not documented | 52 |
| MLT2C1_dup1141 | Uniquely | No | — | — | LTR | ERVL | chr7: 77907836-77908209 | + | 82 | 374 | MAGI2 | Proximal | 0 | Not documented | 53 |
| CR1_Mam_dup822 | Multi/ | No | — | — | LINE | CR1 | chr7: 78050258-78050752 | + | 130 | 495 | MAGI2 | Proximal | 0 | Not documented | 54 |
| | Uniquely | | | | | | | | | | | | | | |
| AluJb_dup55843 | Uniquely | No | — | — | SINE | Alu | chr7: 78050849-78051146 | + | 65 | 298 | MAGI2 | Proximal | 0 | Not documented | 55 |
| L4_dup7842 | Multi/ | No | — | — | LINE | RTE | chr7: 78909661-78909721 | − | 140 | 61 | MAGI2 | Proximal | 0 | Not documented | 56 |
| | Uniquely | | | | | | | | | | | | | | |
| AluJr4_dup7358 | Multi/ | No | — | — | SINE | Alu | chr7: 78909965-78910272 | + | 84 | 308 | MAGI2 | Proximal | 0 | Not documented | 57 |
| | Uniquely | | | | | | | | | | | | | | |
| AluSc_dup14161 | Multi | No | — | — | SINE | Alu | chr7: 78928763-78928889 | + | 29 | 127 | MAGI2 | Proximal | 0 | Not documented | 58 |
| LTR78B_dup1623 | Uniquely | No | — | — | LTR | ERV1 | chr7: 79061843-79062585 | + | 160 | 743 | MAGI2 | Proximal | 0 | Not documented | 59 |
| L2_dup25389 | Multi | No | — | — | LINE | L2 | chr7: 79088912-79089103 | − | 150 | 192 | MAGI2 | Distal | 6023 | Not documented | 60 |
| L3_dup20113 | Multi | No | — | — | LINE | CR1 | chr7: 79090532-79090824 | + | 150 | 293 | MAGI2 | Distal | 7643 | Not documented | 61 |
| L3_dup20114 | Multi/ | No | — | — | LINE | CR1 | chr7: 79091390-79091739 | + | 170 | 350 | MAGI2 | Distal | 8501 | Not documented | 62 |
| | Uniquely | | | | | | | | | | | | | | |
| L3_dup20115 | Multi | No | — | — | LINE | CR1 | chr7: 79091772-79091831 | + | 150 | 60 | MAGI2 | Distal | 8883 | Not documented | 63 |
| L1ME1_dup13154 | Uniquely | No | — | — | LINE | Other L1 | chr7: 8065202-8065987 | + | 110 | 786 | GLCCI1 | Proximal | 0 | Not documented | 64 |
| L1MA2_dup2814 | Multi/ | No | — | — | LINE | Other L1 | chr7: 8067137-8071329 | + | 50 | 4193 | GLCCI1 | Proximal | 0 | Not documented | 65 |
| | Uniquely | | | | | | | | | | | | | | |
| L1ME1_dup13155 | Uniquely | No | — | — | LINE | Other L1 | chr7: 8071350-8074210 | + | 100 | 2861 | GLCCI1 | Proximal | 0 | Not documented | 66 |
| L1PB4_dup2972 | Uniquely | No | — | — | LINE | L1PA\|B\|x | chr7: 8075511-8077740 | + | 110 | 2230 | GLCCI1 | Proximal | 0 | Not documented | 67 |
| L1PB4_dup2973 | Uniquely | No | — | — | LINE | L1PA\|B\|x | chr7: 8077738-8079345 | + | 72 | 1608 | GLCCI1 | Proximal | 0 | Not documented | 68 |
| MER6A_dup484 | Uniquely | No | — | — | DNA | DNA | chr7: 8084550-8085088 | − | 80 | 539 | GLCCI1 | Proximal | 0 | Not documented | 69 |
| MLT2B3_dup1522 | Multi | No | — | — | LTR | ERVL | chr7: 80999052-80999635 | + | 92 | 584 | AC005008.2 | Distal | 170622 | Not documented | 70 |
| AluJo_dup25235 | Uniquely | No | — | — | SINE | Alu | chr7: 8103237-8103556 | − | 92 | 320 | GLCCI1 | Proximal | 0 | Not documented | 71 |
| MSTB_dup3597 | Multi | No | — | — | LTR | ERVL- | chr7: 81040859-81041195 | − | 94 | 337 | AC005008.2 | Distal | 212429 | Not documented | 72 |
| | | | | | | MaLR | | | | | | | | | |
| MIR3_dup39119 | Multi | No | — | — | SINE | MIR | chr7: 81057354-81057525 | − | 140 | 172 | AC005008.2 | Distal | 228924 | Not documented | 73 |
| MER61-int_dup535 | Multi | No | — | — | LTR | ERV1 | chr7: 86952700-86956041 | − | 75 | 3342 | CROT | Distal | 18957 | Not documented | 74 |
| MIRb_dup96253 | Multi/ | No | — | — | SINE | MIR | chr7: 87834631-87834792 | − | 130 | 162 | SRI | Proximal | 0 | Not documented | 4475 |
| | Uniquely | | | | | | | | | | | | | | |
| L1ME4a_dup19055 | Multi/ | No | — | — | LINE | Other L1 | chr7: 87834830-87834942 | − | 120 | 113 | SRI | Proximal | 0 | Not documented | 76 |
| | Uniquely | | | | | | | | | | | | | | |
| L1ME4a_dup19056 | Multi/ | No | — | — | LINE | Other L1 | chr7: 87835051-87835162 | − | 90 | 112 | SRI | Proximal | 0 | Not documented | 77 |
| | Uniquely | | | | | | | | | | | | | | |
| L1PB1_dup6066 | Uniquely | No | — | — | LINE | L1PA\|B\|x | chr7: 91448306-91454839 | − | 49 | 6534 | MTERF | Proximal | 0 | Not documented | 78 |
| MLT1D_dup8528 | Uniquely | No | — | — | LTR | ERVL- | chr7: 91502432-91502912 | − | 95 | 481 | MTERF | Proximal | 0 | Not documented | 79 |
| | | | | | | MaLR | | | | | | | | | |
| AluSg7_dup3312 | Multi | No | — | — | SINE | AIL | chr7: 91741862-91742161 | + | 29 | 300 | CYP51A1 | Proximal | 0 | Not documented | 80 |
| AluSx1_dup41961 | Multi/ | No | — | — | SINE | Alu | chr7: 91829672-91829965 | − | 53 | 294 | KRIT1 | Proximal | 0 | Not documented | 81 |
| | Uniquely | | | | | | | | | | | | | | |
| AluSx4_dup2312 | Uniquely | No | — | — | SINE | Alu | chr7: 91869022-91869317 | − | 40 | 296 | KRIT1 | Proximal | 0 | Not documented | 82 |
| MER20B_dup1853 | Multi | No | — | — | DNA | DNA | chr7: 91874185-91874340 | + | 120 | 156 | KRIT1 | Proximal | 0 | Not documented | 83 |
| L2_dup25690 | Multi/ | No | — | — | LINE | L2 | chr7: 91978039-91978630 | − | 140 | 592 | ANKIB1 | Proximal | 0 | Not documented | 84 |
| | Uniquely | | | | | | | | | | | | | | |
| AluSx3_dup11898 | Multi | No | — | — | SINE | Alu | chr7: 92085903-92085940 | + | 72 | 38 | GATAD1 | Proximal | 0 | Not documented | 85 |
| AluSx3_dup11899 | Multi | No | — | — | SINE | Alu | chr7: 92085944-92086004 | + | 100 | 61 | GATAD1 | Proximal | 0 | Not documented | 86 |
| AluSx_dup54449 | Multi | No | — | — | SINE | Alu | chr7: 92086169-92086495 | + | 49 | 327 | GATAD1 | Proximal | 0 | Not documented | 87 |
| AluSx_dup54450 | Multi | No | — | — | SINE | Alu | chr7: 92086496-92086796 | + | 42 | 301 | GATAD1 | Proximal | 0 | Not documented | 88 |
| AluSz_dup37752 | Multi | No | — | — | SINE | Alu | chr7: 92087169-92087470 | + | 53 | 302 | GATAD1 | Proximal | 0 | Not documented | 89 |
| AluSg_dup16537 | Multi | No | — | — | SINE | Alu | chr7: 92166399-92166683 | − | 45 | 285 | RBM48 | Proximal | 0 | Not documented | 90 |
| L2a_dup76938 | Uniquely | No | — | — | LINE | L2 | chr7: 92192710-92193609 | + | 140 | 900 | FAM133B | Proximal | 0 | Not documented | 91 |
| L1ME2z_dup3084 | Multi | No | — | — | LINE | Other L1 | chr7: 92774994-92775069 | − | 78 | 76 | SAMD9L | Proximal | 0 | Not documented | 92 |
| L2a_dup76971 | Multi/ | No | — | — | LINE | L2 | chr7: 92775142-92775894 | + | 130 | 753 | SAMD9L | Proximal | 0 | Not documented | 93 |
| | Uniquely | | | | | | | | | | | | | | |
| L1PB1_dup6082 | Uniquely | No | — | — | LINE | L1PA\|B\|x | chr7: 94805444-94810484 | + | 42 | 5041 | PPP1R9A | Proximal | 0 | EVE annotated | 94 |
| L1PB1_dup6089 | Uniquely | No | — | — | LINE | L1PA\|B\|x | chr7: 94858640-94860069 | + | 52 | 1430 | PPP1R9A | Proximal | 0 | Not documented | 95 |
| HAL1_dup12515 | Multi/ | No | — | — | LINE | Other L1 | chr7: 95041990-95042071 | + | 120 | 82 | PON2 | Proximal | 0 | Not documented | 96 |
| | Uniquely | | | | | | | | | | | | | | |
| L2a_dup77081 | Multi | No | — | — | LINE | L2 | chr7: 95042103-95042147 | + | 50 | 45 | PON2 | Proximal | 0 | Not documented | 97 |
| L1M5_dup28100 | Multi/ | No | — | — | LINE | Other L1 | chr7: 95042149-95042308 | − | 150 | 160 | PON2 | Proximal | 0 | Not documented | 98 |
| | Uniquely | | | | | | | | | | | | | | |
| AluJr_dup30054 | Multi | No | — | — | SINE | Alu | chr7: 95042309-95042612 | + | 75 | 304 | PON2 | Proximal | 0 | Not documented | 99 |
| L1M5_dup28101 | Multi/ | No | — | — | LINE | Other L1 | chr7: 95042615-95043029 | − | 130 | 415 | PON2 | Proximal | 0 | Not documented | 4500 |
| | Uniquely | | | | | | | | | | | | | | |
| L1MB3_dup6860 | Multi/ | No | — | — | LINE | Other L1 | chr7: 95043362-95043451 | − | 65 | 90 | PON2 | Proximal | 0 | Not documented | 1 |
| | Uniquely | | | | | | | | | | | | | | |
| L3b_dup3237 | Multi/ | No | — | — | LINE | CR1 | chr7: 95044162-95044407 | − | 120 | 246 | PON2 | Proximal | 0 | Not documented | 2 |
| | Uniquely | | | | | | | | | | | | | | |
| AluSq_dup8517 | Multi/ | No | — | — | SINE | Alu | chr7: 95044984-95045294 | + | 40 | 311 | PON2 | Proximal | 0 | Not documented | 3 |
| | Uniquely | | | | | | | | | | | | | | |
| AluSx_dup54602 | Multi | No | — | — | SINE | Alu | chr7: 97552097-97552399 | − | 54 | 303 | ASNS | Distal | 50244 | Not documented | 4 |
| LTR5A_dup109 | Uniquely | No | — | — | LTR | ERVK | chr7: 97570329-97571358 | − | 45 | 1030 | 0CM2 | Distal | 42639 | Not documented | 5 |
| SATR2_dup108 | Uniquely | No | — | — | Other | Other | chr7: 97593719-97595337 | + | 79 | 1619 | OCM2 | Distal | 18660 | Not documented | 6 |
| | | | | | | Repeats | | | | | | | | | |
| AluY_dup48713 | Multi | No | — | — | SINE | Alu | chr7: 97599682-97599990 | − | 19 | 309 | OCM2 | Distal | 14007 | Not documented | 7 |
| AluJr_dup30100 | Multi | No | — | — | SINE | Alu | chr7: 97600536-97600853 | − | 57 | 318 | OCM2 | Distal | 13144 | Not documented | 8 |
| AluSx1_dup42107 | Multi | No | — | — | SINE | Alu | chr7: 97845871-97846175 | − | 33 | 305 | TECPR1 | Proximal | 0 | Not documented | 9 |
| L1MD2_dup4539 | Multi | No | — | — | LINE | Other L1 | chr7: 98730568-98730806 | + | 100 | 239 | SMURF1 | Proximal | 0 | Not documented | 10 |
| L1MD2_dup4540 | Multi | No | — | — | LINE | Other L1 | chr7: 98730879-98731024 | + | 100 | 146 | SMURF1 | Proximal | 0 | Not documented | 11 |
| MIR3_dup39572 | Multi/ | No | — | — | SINE | MIR | chr7: 99007610-99007772 | − | 110 | 163 | BUD31 | Proximal | 0 | Not documented | 12 |
| | Uniquely | | | | | | | | | | | | | | |
| LTR13_dup142 | Multi/ | No | — | — | LTR | ERVK | chr7: 99012668-99013646 | − | 20 | 979 | BUD31 | Proximal | 0 | Not documented | 13 |
| | Uniquely | | | | | | | | | | | | | | |
| Charlie12_dup6 | Uniquely | No | — | — | DNA | DNA | chr7: 99265999-99268740 | + | 53 | 2742 | CYP3A5 | Proximal | 0 | Not documented | 14 |
| MIRb_dup96915 | Multi | No | — | — | SINE | MIR | chr7: 99488839-99489025 | + | 150 | 187 | TRIM4 | Proximal | 0 | Not documented | 15 |
| MLT1L_dup5595 | Multi/ | No | — | — | LTR | ERVL- | chr7: 99489317-99489404 | − | 88 | 88 | TRIM4 | Proximal | 0 | Not documented | 16 |
| | Uniquely | | | | | MaLR | | | | | | | | | |
| AluSx1_dup42311 | Multi | No | — | — | SINE | Alu | chr7: 99634058-99634364 | + | 41 | 307 | ZKSCAN1 | Proximal | 0 | Not documented | 17 |
| AluSx3_dup11970 | Multi | No | — | — | SINE | Alu | chr7: 99635359-99635654 | + | 35 | 296 | ZKSCAN1 | Proximal | 0 | Not documented | 18 |
| AluSc8_dup8813 | Multi | No | — | — | SINE | Alu | chr7: 99636337-99636631 | + | 43 | 295 | ZKSCAN1 | Proximal | 0 | Not documented | 19 |
| AluSx1_dup42312 | Multi | No | — | — | SINE | Alu | chr7: 99636653-99636958 | + | 42 | 30€ | ZKSCAN1 | Proximal | 0 | Not documented | 20 |
| MIR3_dup39588 | Multi | No | — | — | SINE | MIR | chr7: 99722818-99722923 | + | 180 | 106 | CNPY4 | Proximal | 0 | Not documented | 21 |
| AluSg_dup16662 | Uniquely | No | — | — | SINE | Alu | chr7: 99745477-99745770 | + | 32 | 294 | LAMTOR4 | Proximal | 76. | Not documented | 22 |
| MLT1A0_dup9536 | Uniquely | No | — | — | LTR | ERVL- | chr7: 99810604-99810933 | − | 91 | 330 | STAG3 | Proximal | 0 | Not documented | 23 |
| | | | | | | MaLR | | | | | | | | | |
| AluSx_dup54916 | Multi | No | — | — | SINE | Alu | chr7: 99905671-99905983 | + | 51 | 313 | SPDYE3 | Proximal | 0 | Not documented | 24 |
| AluJb_dup56664 | Multi | No | — | — | SINE | Alu | chr7: 99907036-99907373 | + | 75 | 338 | SPDYE3 | Proximal | 0 | Not documented | 4525 |
| AluJb_dup56665 | Multi | No | — | — | SINE | Alu | chr7: 99908422-99908759 | + | 77 | 338 | SPDYE3 | Proximal | 0 | Not documented | 26 |
| AluJb_dup56666 | Multi | No | — | — | SINE | Alu | chr7: 99909812-99910149 | + | 81 | 338 | SPDYE3 | Proximal | 0 | Not documented | 27 |
| AluJb_dup56667 | Multi | No | — | — | SINE | Alu | chr7: 99911202-99911537 | + | 76 | 336 | SPDYE3 | Proximal | 0 | Not documented | 28 |
| AluJo_dup27929 | Multi | No | — | — | SINE | Alu | chr7: 99912594-99912933 | + | 84 | 340 | SPDYE3 | Proximal | 0 | Not documented | 29 |
| MSTA_dup8737 | Multi/ | No | — | — | LTR | ERVL- | chr7: 99923235-99923621 | − | 67 | 387 | SPDYE3 | Distal | 3417 | Not documented | 30 |
| | Uniquely | | | | | MaLR | | | | | | | | | |
| AluSx1_dup42359 | Multi | No | — | — | SINE | Alu | chr7: 99932345-99932645 | − | 50 | 301 | PILRB | Proximal | 1093 | Not documented | 31 |
| LTR49_dup646 | Multi | No | — | — | LTR | ERV1 | chr7: 99946988-99947533 | − | 110 | 546 | PILRB | Proximal | 0 | Not documented | 32 |
| AluJb_dup56673 | Multi | No | — | — | SINE | Alu | chr7: 99948857-99948924 | − | 54 | 68 | PILRB | Proximal | 0 | Not documented | 33 |
| L1MD_dup3702 | Multi/ | No | — | — | LINE | Other L1 | chr7: 99952749-99952855 | − | 120 | 107 | PILRB | Proximal | 0 | Not documented | 34 |
| | Uniquely | | | | | | | | | | | | | | |
| MLT1J2_dup3124 | Multi/ | No | — | — | LTR | ERVL- | chr7: 99964960-99965296 | + | 140 | 337 | PILRB | Proximal | 0 | Not documented | 35 |
| | Uniquely | | | | | MaLR | | | | | | | | | |
| Charlie1b_dup1612 | Multi | No | — | — | DNA | DNA | chr8: 100008859-100009290 | − | 69 | 432 | VPS13B | Distal | 16205 | Not documented | 36 |
| LTR56_dup499 | Multi | No | — | — | LTR | ERV1 | chr8: 100904274-100904410 | − | 65 | 137 | COX6C | Proximal | 0 | Not documented | 37 |
| MER44A_dup1267 | Multi/ | No | — | — | DNA | DNA | chr8: 101165634-101165961 | − | 65 | 328 | POLR2K | Proximal | 0 | Not documented | 38 |
| | Uniquely | | | | | | | | | | | | | | |
| MER5A_dup17355 | Multi | No | — | — | DNA | DNA | chr8: 101166150-101166219 | − | 120 | 70 | POLR2K | Proximal | 0 | Not documented | 39 |
| MER34B-int_dup391 | Multi | No | — | — | LTR | ERV1 | chr8: 101931930-101932247 | − | 49 | 318 | YWHAZ | Proximal | 0 | Not documented | 40 |
| AluJb_dup64043 | Multi | No | — | — | SINE | Alu | chr8: 102218732-102218909 | + | 55 | 178 | ZNF706 | Proximal | 312 | Not documented | 41 |
| MER74A_dup716 | Uniquely | No | — | — | LTR | ERVL | chr8: 10328116-10328624 | + | 84 | 509 | MSRA | Distal | 41716 | Not documented | 42 |
| AluJb_dup64080 | Multi | No | — | — | SINE | Alu | chr8: 103365996-103366276 | + | 76 | 281 | UBR5 | Proximal | 0 | Not documented | 43 |
| L1MC4a_dup13492 | Multi | No | — | — | LINE | Other L1 | chr8: 103412264-103412358 | + | 110 | 95 | UBR5 | Proximal | 0 | Not documented | 44 |
| AluJo_dup31870 | Multi | No | — | — | SINE | Alu | chr8: 103412367-103412538 | + | 83 | 172 | UBR5 | Proximal | 0 | Not documented | 45 |
| ERVL-E-int_{—} | Uniquely | No | — | — | LTR | ERVL | chr8: 10344122-10345075 | + | 130 | 954 | PRSS55 | Distal | 37982 | Not documented | 46 |
| dup4615 | | | | | | | | | | | | | | | |
| L1MC4a_dup13493 | Uniquely | No | — | — | LINE | Other L1 | chr8: 103658724-103659397 | + | 91 | 674 | KLF10 | Proximal | 1611 | Not documented | 47 |
| MLT1F2_dup2929 | Uniquely | No | — | — | LTR | ERVL- | chr8: 103892119-103892274 | + | 81 | 15€ | AZIN1 | Proximal | 0 | Not documented | 48 |
| | | | | | | MaLR | | | | | | | | | |
| MIRb_dup106707 | Multi | No | — | — | SINE | MIR | chr8: 104178040-104178159 | + | 130 | 120 | BAALC | Proximal | 0 | Not documented | 49 |
| L2b_dup46482 | Multi | No | — | — | LINE | L2 | chr8: 104241740-104241800 | − | 140 | 61 | BAALC | Proximal | 0 | Not documented | 4550 |
| L1PA7_dup6921 | Multi | No | — | — | LINE | L1PA\|B\|x | chr8: 109133337-109135710 | − | 45 | 2374 | RSPO2 | Distal | 37425 | Not documented | 51 |
| L2b_dup46643 | Multi | No | — | — | LINE | L2 | chr8: 109153060-109153287 | + | 170 | 228 | RSPO2 | Distal | 57148 | Not documented | 52 |
| AluJb_dup59978 | Multi | No | — | — | SINE | Alu | chr8: 11644138-11644447 | − | 92 | 310 | NEIL2 | Proximal | 0 | Not documented | 53 |
| THE1C-int_dup983 | Uniquely | No | — | — | LTR | ERVL- | chr8: 116483761-116485357 | − | 53 | 1597 | TRPS1 | Proximal | 0 | Not documented | 54 |
| | | | | | | MaLR | | | | | | | | | |
| AluJb_dup64526 | Multi | No | — | — | SINE | Alu | chr8: 118937826-118938168 | + | 69 | 343 | EXT1 | Proximal | 0 | Not documented | 55 |
| AluJb_dup64538 | Uniquely | No | — | — | SINE | Alu | chr8: 119102387-119102668 | + | 79 | 282 | EXT1 | Proximal | 0 | Not documented | 56 |
| AluSx3_dup13519 | Uniquely | No | — | — | SINE | Alu | chr8: 120143460-120143762 | − | 53 | 303 | COLEC10 | Distal | 24640 | Not documented | 57 |
| L1M2_dup4064 | Multi | No | — | — | LINE | Other L1 | chr8: 12414108-12416270 | + | 76 | 2163 | FAM86B2 | Distal | 120194 | Not documented | 58 |
| LTR5A_dup154 | Multi | No | — | — | LTR | ERVK | chr8: 12423489-12424512 | + | 38 | 1024 | FAM86B2 | Distal | 129575 | Not documented | 59 |
| L1ME1_dup16014 | Uniquely | No | — | — | LINE | Other L1 | chr8: 126330854-126332136 | + | 90 | 1283 | NSMCE2 | Proximal | 0 | Not documented | 60 |
| L1MC3_dup6595 | Uniquely | No | — | — | LINE | Other L1 | chr8: 129009846-129010765 | − | 91 | 920 | TMEM75 | Distal | 49256 | Not documented | 61 |
| L2c_dup67083 | Multi | No | — | — | LINE | L2 | chr8: 12940890-12941010 | − | 120 | 121 | DLC1 | Proximal | 0 | Not documented | 62 |
| MIRc_dup51938 | Multi | No | — | — | SINE | MIR | chr8: 137877089-137877309 | + | 120 | 221 | KHDRBS3 | Distal | 1208125 | Not documented | 63 |
| L1PA7_dup7091 | Uniquely | No | — | — | LINE | L1PA\|B\|x | chr8: 141912434-141915275 | − | 34 | 2842 | PTK2 | Proximal | 0 | EVE annotated | 64 |
| MIR3_dup45519 | Multi | No | — | — | SINE | MIR | chr8: 143817956-143818054 | − | 97 | 99 | THEM6 | Proximal | 0 | Not documented | 65 |
| L2b_dup48422 | Multi/ | No | — | — | LINE | L2 | chr8: 144363090-144363196 | + | 170 | 107 | GLI4 | Distal | 3990 | Not documented | 66 |
| | Uniquely | | | | | | | | | | | | | | |
| LTR50_dup1387 | Multi | No | — | — | LTR | ERVL | chr8: 144363779-144364006 | − | 120 | 228 | GLI4 | Distal | 4679 | Not documented | 67 |
| MLT1E2_dup2031 | Multi | No | — | — | LTR | ERVL- | chr8: 144916197-144916760 | + | 110 | 564 | NRBP2 | Proximal | 0 | Not documented | 68 |
| | | | | | | MaLR | | | | | | | | | |
| L2_dup30041 | Multi | No | — | — | LINE | L2 | chr8: 144916867-144917273 | − | 140 | 407 | NRBP2 | Proximal | 0 | Not documented | 69 |
| MIR_dup88702 | Multi | No | — | — | SINE | MIR | chr8: 145158193-145158461 | − | 140 | 269 | SHARPIN | Proximal | 0 | Not documented | 70 |
| MER115_dup1390 | Multi | No | — | — | DNA | DNA | chr8: 145750473-145750694 | − | 130 | 222 | LRRC24 | Proximal | 0 | Not documented | 71 |
| L1MC1_dup7227 | Uniquely | No | — | — | LINE | Other L1 | chr8: 145975397-145976231 | − | 81 | 835 | ZNF251 | Proximal | 0 | Not documented | 72 |
| L2b_dup48437 | Multi | No | — | — | LINE | L2 | chr8: 146075117-146075646 | + | 130 | 530 | COMMD5 | Proximal | 0 | Not documented | 73 |
| L2a_dup89099 | Multi/ | No | — | — | LINE | L2 | chr8: 146280335-146280450 | + | 130 | 116 | C8orf33 | Proximal | 0 | Not documented | 74 |
| | Uniquely | | | | | | | | | | | | | | |
| LTR13_dup183 | Multi/ | No | — | — | LTR | ERVK | chr8: 146280512-146281504 | + | 25 | 993 | C8orf33 | Proximal | 0 | Not documented | 4575 |
| | Uniquely | | | | | | | | | | | | | | |
| MIR3_dup41719 | Uniquely | No | — | — | SINE | MIR | chr8: 17089565-17089730 | + | 140 | 166 | CNOT7 | Proximal | 0 | Not documented | 76 |
| LTR5B_dup156 | Multi | No | — | — | LTR | ERVK | chr8: 23148997-23149045 | − | 65 | 49 | R3HCC1 | Proximal | 0 | Not documented | 77 |
| MIRb_dup101912 | Multi/ | No | — | — | SINE | MIR | chr8: 26362710-26362880 | − | 120 | 171 | PNMA2 | Proximal | 0 | Not documented | 78 |
| | Uniquely | | | | | | | | | | | | | | |
| AluSx1_dup45696 | Multi/ | No | — | — | SINE | Alu | chr8: 26363227-26363509 | + | 35 | 283 | PNMA2 | Proximal | 0 | Not documented | 79 |
| | Uniquely | | | | | | | | | | | | | | |
| MIRb_dup101913 | Multi/ | No | — | — | SINE | MIR | chr8: 26363602-26363836 | + | 130 | 235 | PNMA2 | Proximal | 0 | Not documented | 80 |
| | Uniquely | | | | | | | | | | | | | | |
| L2c_dup67707 | Multi/ | No | — | — | LINE | L2 | chr8: 26364224-26364392 | + | 110 | 169 | PNMA2 | Proximal | 0 | Not documented | 81 |
| | Uniquely | | | | | | | | | | | | | | |
| AluJb_dup60648 | Multi/ | No | — | — | SINE | Alu | chr8: 26364663-26364979 | + | 75 | 317 | PNMA2 | Proximal | 0 | Not documented | 82 |
| | Uniquely | | | | | | | | | | | | | | |
| MamGyp-int_dup157 | Multi/ | No | — | — | LTR | Gypsy | chr8: 26365257-26366372 | − | 150 | 1116 | PNMA2 | Proximal | 0 | Not documented | 83 |
| | Uniquely | | | | | | | | | | | | | | |
| MIRc_dup47827 | Multi | No | — | — | SINE | MIR | chr8: 26514443-26514531 | − | 140 | 89 | DPYSL2 | Proximal | 0 | Not documented | 84 |
| MER103C_dup4447 | Multi/ | No | — | — | DNA | DNA | chr8: 27465719-27465833 | + | 89 | 115 | CLU | Proximal | 0 | Not documented | 85 |
| | Uniquely | | | | | | | | | | | | | | |
| L2a_dup81859 | Multi/ | No | — | — | LINE | L2 | chr8: 27466092-27466216 | + | 150 | 125 | CLU | Proximal | 0 | Not documented | 86 |
| | Uniquely | | | | | | | | | | | | | | |
| L1ME4a_dup20528 | Multi | No | — | — | LINE | Other L1 | chr8: 27471189-27471380 | − | 140 | 192 | CLU | Proximal | 0 | Not documented | 87 |
| MIRc_dup47869 | Multi/ | No | — | — | SINE | MIR | chr8: 27471799-27471947 | + | 140 | 149 | CLU | Proximal | 0 | Not documented | 88 |
| | Uniquely | | | | | | | | | | | | | | |
| Charlie7_dup1894 | Multi | No | — | — | DNA | DNA | chr8: 27534105-27534250 | + | 130 | 146 | SCARA3 | Proximal | 0 | Not documented | 89 |
| MARNA_dup1681 | Multi | No | — | — | DNA | DNA | chr8: 28203321-28203713 | + | 140 | 393 | ZNF395 | Proximal | 0 | Not documented | 90 |
| AluSz6_dup19773 | Multi | No | — | — | SINE | Alu | chr8: 28831322-28831629 | − | 38 | 308 | HMBOX1 | Proximal | 0 | Not documented | 91 |
| AluJr_dup33021 | Multi | No | — | — | SINE | Alu | chr8: 30013363-30013637 | − | 66 | 275 | LEPROTL1 | Proximal | 0 | Not documented | 92 |
| MLT1J_dup7381 | Uniquely | No | — | — | LTR | ERVL- | chr8: 30022390-30022591 | − | 100 | 202 | DCTN6 | Proximal | 0 | Not documented | 93 |
| | | | | | | MaLR | | | | | | | | | |
| L2a_dup81997 | Multi | No | — | — | LINE | L2 | chr8: 30210951-30211032 | + | 83 | 82 | RBPMS | Distal | 30913 | Not documented | 94 |
| LTR33_dup4479 | Multi | No | — | — | LTR | ERVL | chr8: 32785298-32785732 | + | 150 | 435 | NRG1 | Distal | 162751 | Not documented | 95 |
| MIR_dup81599 | Multi | No | — | — | SINE | MIR | chr8: 32901860-32901996 | − | 110 | 137 | NRG1 | Distal | 279313 | Not documented | 96 |
| L1MB4_dup3958 | Multi | No | — | — | LINE | Other L1 | chr8: 33358567-33358701 | + | 70 | 135 | MAK16 | Proximal | 0 | Not documented | 97 |
| AluJo_dup30460 | Multi | No | — | — | SINE | Alu | chr8: 37636194-37636451 | + | 63 | 258 | PROSC | Proximal | 0 | Not documented | 98 |
| L1MC4a_dup12931 | Multi | No | — | — | LINE | Other L1 | chr8: 38069885-38070483 | − | 75 | 599 | BAG4 | Proximal | 0 | Not documented | 99 |
| AluJr_dup33380 | Uniquely | No | — | — | SINE | Alu | chr8: 38279925-38280219 | − | 65 | 295 | FGFR1 | Proximal | 0 | Not documented | 4600 |
| L1P2_dup768 | Uniquely | No | — | — | LINE | L1PA\|B\|x | chr8: 39000001-39001875 | − | 40 | 1875 | ADAM32 | Proximal | 0 | Not documented | 1 |
| AluY_dup52614 | Multi | No | — | — | SINE | Alu | chr8: 40139543-40139847 | − | 28 | 305 | C8orf4 | Distal | 126723 | Not documented | 2 |
| MER6_dup400 | Uniquely | No | — | — | DNA | DNA | chr8: 42033765-42034481 | + | 72 | 717 | PLAT | Proximal | 0 | Not documented | 3 |
| MER4A_dup485 | Uniquely | No | — | — | LTR | ERV1 | chr8: 42043555-42044204 | + | 52 | 550 | PLAT | Proximal | 0 | Not documented | 4 |
| MIRc_dup48468 | Uniquely | No | — | — | SINE | MIR | chr8: 42048476-42048622 | + | 140 | 147 | PLAT | Proximal | 0 | Not documented | 5 |
| AluSx3_dup13029 | Multi | No | — | — | SINE | Alu | chr8: 48889544-48889845 | + | 42 | 302 | MCM4 | Proximal | 0 | Not documented | 6 |
| AluJb_dup61801 | Multi/ | No | — | — | SINE | Alu | chr8: 48890052-48890318 | − | 89 | 267 | MCM4 | Proximal | 0 | Not documented | 7 |
| | Uniquely | | | | | | | | | | | | | | |
| Tigger2a_dup1649 | Multi/ | No | — | — | DNA | DNA | chr8: 51556816-51557279 | + | 67 | 464 | SNTG1 | Proximal | 0 | Not documented | 8 |
| | Uniquely | | | | | | | | | | | | | | |
| LTR17_dup439 | Uniquely | No | — | — | LTR | ERV1 | chr8: 55060881-55061641 | − | 52 | 761 | MRPL15 | Proximal | 421 | Not documented | 9 |
| MIR_dup82455 | Multi | No | — | — | SINE | MIR | chr8: 56652218-56652274 | − | 110 | 57 | TMEM68 | Proximal | 0 | Not documented | 10 |
| L1MC4_dup14087 | Multi | No | — | — | LINE | Other L1 | chr8: 56692940-56693861 | − | 110 | 922 | TGS1 | Proximal | 0 | Not documented | 11 |
| Tigger6a_dup389 | Uniquely | No | — | — | DNA | DNA | chr8: 59018631-59019628 | + | 93 | 998 | FAM110B | Proximal | 0 | Not documented | 12 |
| MLT1D-int_dup354 | Multi | No | — | — | LTR | ERVL- | chr8: 59361978-59363260 | − | 120 | 1283 | UBXN2B | Proximal | 0 | Not documented | 13 |
| | | | | | | MaLR | | | | | | | | | |
| L1MB3_dup7726 | Multi | No | — | — | LINE | Other L1 | chr8: 60882514-60882963 | − | 65 | 450 | CA8 | Distal | 216944 | Not documented | 14 |
| LTR47B_dup187 | Multi | No | — | — | LTR | ERVL | chr8: 60883038-60883410 | + | 83 | 373 | CA8 | Distal | 216497 | Not documented | 15 |
| L1PA7_dup6690 | Uniquely | No | — | — | LINE | L1PA\|B\|x | chr8: 63151475-63155248 | − | 55 | 3774 | NKAIN3 | Distal | 5903 | EVE annotated | 16 |
| L1PA11_dup2013 | Uniquely | No | — | — | LINE | L1PA\|B\|x | chr8: 63769684-63771882 | − | 97 | 2199 | NKAIN3 | Proximal | 0 | Not documented | 17 |
| L1ME2_dup6257 | Multi | No | — | — | LINE | Other L1 | chr8: 63878984-63879213 | − | 110 | 230 | NKAIN3 | Proximal | 0 | Not documented | 18 |
| MIRc_dup49014 | Multi | No | — | — | SINE | MIR | chr8: 67428966-67429105 | − | 100 | 140 | C8orf46 | Proximal | 0 | Not documented | 19 |
| MADE2_dup1310 | Multi | No | — | — | DNA | DNA | chr8: 67429317-67429391 | − | 73 | 75 | C8orf46 | Proximal | 0 | Not documented | 20 |
| MIRb_dup104133 | Multi | No | — | — | SINE | MIR | chr8: 67429503-67429679 | − | 140 | 177 | C8orf46 | Proximal | 0 | Not documented | 21 |
| MER117_dup2090 | Multi | No | — | — | DNA | DNA | chr8: 67429735-67429823 | + | 150 | 89 | C8orf46 | Proximal | 0 | Not documented | 22 |
| FLAM_A_dup7518 | Multi | No | — | — | SINE | Alu | chr8: 67430423-67430549 | − | 82 | 127 | C8orf46 | Proximal | 0 | Not documented | 23 |
| AluSx_dup60743 | Uniquely | No | — | — | SINE | Alu | chr8: 67590049-67590346 | + | 49 | 298 | C8orf44 | Proximal | 0 | Not documented | 24 |
| L1PB2_dup1357 | Multi | No | — | — | LINE | L1PA\|B\|x | chr8: 69158480-69160478 | − | 60 | 1999 | PREX2 | Distal | 9216 | Not documented | 4625 |
| L1MC1_dup6892 | Multi | No | — | — | LINE | Other L1 | chr8: 69258514-69259177 | − | 65 | 664 | C8orf34 | Proximal | 0 | Not documented | 26 |
| MLT1E1A_dup1683 | Multi | No | — | — | LTR | ERVL- | chr8: 69353358-69353644 | − | 89 | 287 | C8orf34 | Proximal | 0 | Not documented | 27 |
| | | | | | | MaLR | | | | | | | | | |
| THE1B_dup11147 | Multi | No | — | — | LTR | ERVL- | chr8: 69574610-69574951 | + | 66 | 342 | C8orf34 | Proximal | 0 | Not documented | 28 |
| | | | | | | MaLR | | | | | | | | | |
| AluSc_dup15857 | Multi | No | — | — | SINE | Alu | chr8: 74235889-74236184 | − | 41 | 296 | RDH10 | Proximal | 0 | Not documented | 29 |
| L1ME2z_dup3405 | Multi | No | — | — | LINE | Other L1 | chr8: 74876562-74876749 | + | 110 | 188 | TCEB1 | Proximal | 0 | Not documented | 30 |
| FLAM_C_dup9762 | Multi | No | — | — | SINE | Alu | chr8: 74876750-74876884 | + | 67 | 135 | TCEB1 | Proximal | 0 | Not documented | 31 |
| AluSz_dup42650 | Multi | No | — | — | SINE | Alu | chr8: 74891278-74891563 | − | 62 | 286 | TMEM70 | Proximal | 0 | Not documented | 32 |
| AluJr_dup34280 | Multi | No | — | — | SINE | Alu | chr8: 77734689-77734975 | − | 70 | 287 | ZFHX4 | Proximal | 0 | Not documented | 33 |
| Tigger1_dup6314 | Multi | No | — | — | DNA | DNA | chr8: 77900534-77901148 | − | 60 | 615 | PEX2 | Proximal | 0 | Not documented | 34 |
| L1MA6_dup2734 | Uniquely | No | — | — | LINE | Other L1 | chr8: 79738850-79740520 | + | 70 | 1671 | IL7 | Distal | 21093 | Not documented | 35 |
| LTR6A_dup91 | Multi | No | — | — | LTR | ERV1 | chr8: 81471111-81471140 | − | 15 | 30 | ZBTB10 | Distal | 32612 | Not documented | 36 |
| L1MA4A_dup3122 | Uniquely | No | — | — | LINE | Other L1 | chr8: 82293281-82299554 | − | 95 | 6274 | PMP2 | Distal | 53008 | Not documented | 37 |
| L1MC4_dup14332 | Multi/ | No | — | — | LINE | Other L1 | chr8: 82352797-82352912 | − | 84 | 116 | PMP2 | Proximal | 0 | Not documented | 38 |
| | Uniquely | | | | | | | | | | | | | | |
| L1MC4_dup14333 | Multi/ | No | — | — | LINE | Other L1 | chr8: 82353037-82353755 | − | 110 | 719 | PMP2 | Proximal | 0 | Not documented | 39 |
| | Uniquely | | | | | | | | | | | | | | |
| L1MC4_dup14334 | Multi/ | No | — | — | LINE | Other L1 | chr8: 82353851-82354515 | − | 110 | 665 | PMP2 | Proximal | 0 | Not documented | 40 |
| | Uniquely | | | | | | | | | | | | | | |
| AluY_dup53900 | Multi/ | No | — | — | SINE | Alu | chr8: 82357449-82357757 | − | 24 | 309 | PMP2 | Proximal | 0 | Not documented | 41 |
| | Uniquely | | | | | | | | | | | | | | |
| Tigger4_dup784 | Multi | No | — | — | DNA | DNA | chr8: 87484608-87485321 | + | 49 | 714 | RMDN1 | Proximal | 0 | Not documented | 42 |
| MIRb_dup105415 | Multi | No | — | — | SINE | MIR | chr8: 87527605-87527786 | − | 180 | 182 | CPNE3 | Proximal | 0 | Not documented | 43 |
| L1MD1_dup3298 | Uniquely | No | — | — | LINE | Other L1 | chr8: 90587181-90590808 | − | 82 | 3628 | RIPK2 | Distal | 179168 | Not documented | 44 |
| AluSx1_dup47822 | Uniquely | No | — | — | SINE | Alu | chr8: 94812130-94812438 | − | 65 | 309 | TMEM67 | Proximal | 0 | Not documented | 45 |
| MIR_dup84500 | Uniquely | No | — | — | SINE | MIR | chr8: 94812754-94812946 | − | 120 | 193 | TMEM67 | Proximal | 0 | Not documented | 46 |
| AluSq2_dup24475 | Multi/ | No | — | — | SINE | Alu | chr8: 95269425-95269709 | − | 50 | 285 | GEM | Proximal | 0 | Not documented | 47 |
| | Uniquely | | | | | | | | | | | | | | |
| MIRb_dup105956 | Multi/ | No | — | — | SINE | MIR | chr8: 95270755-95270995 | + | 130 | 241 | GEM | Proximal | 0 | Not documented | 48 |
| | Uniquely | | | | | | | | | | | | | | |
| MIRb_dup105957 | Multi | No | — | — | SINE | MIR | chr8: 95271025-95271201 | + | 150 | 177 | GEM | Proximal | 0 | Not documented | 49 |
| AluJo_dup31616 | Uniquely | No | — | — | SINE | Alu | chr8: 95273587-95273880 | − | 50 | 294 | GEM | Proximal | 0 | Not documented | 4650 |
| MSTD-int_dup369 | Uniquely | No | — | — | LTR | ERVL- | chr8: 97334889-97336641 | − | 100 | 1753 | PTDSS1 | Proximal | 0 | Not documented | 51 |
| | | | | | | MaLR | | | | | | | | | |
| L1PA7_dup7526 | Uniquely | No | — | — | LINE | L1PA\|B\|x | chr9: 108290004-108292064 | + | 33 | 2061 | FSD1L | Proximal | 0 | EVE annotated | 52 |
| MER91A_dup1919 | Multi | No | — | — | DNA | DNA | chr9: 111680562-111680726 | + | 130 | 165 | IKBKAP | Proximal | 0 | Not documented | 53 |
| MER8_dup1160 | Multi/ | No | — | — | DNA | DNA | chr9: 111702345-111702580 | + | 110 | 236 | FAM206A | Proximal | 0 | Not documented | 54 |
| | Uniquely | | | | | | | | | | | | | | |
| MLT1B_dup9897 | Multi | No | — | — | LTR | ERVL- | chr9: 115233533-115233856 | + | 86 | 324 | C9orf147 | Proximal | 0 | Not documented | 55 |
| | | | | | | MaLR | | | | | | | | | |
| AluJr_dup37473 | Multi | No | — | — | SINE | Alu | chr9: 115233860-115233943 | + | 60 | 84 | C9orf147 | Proximal | 0 | Not documented | 56 |
| MIR3_dup48423 | Multi | No | — | — | SINE | MIR | chr9: 115234198-115234283 | − | 120 | 86 | C9orf147 | Proximal | 0 | Not documented | 57 |
| LTR54B_dup476 | Multi | No | — | — | LTR | ERV1 | chr9: 115234361-115234715 | + | 73 | 355 | C9orf147 | Proximal | 0 | Not documented | 58 |
| L3_dup25257 | Multi | No | — | — | LINE | CR1 | chr9: 115449615-115449667 | − | 140 | 53 | INIP | Proximal | 0 | Not documented | 59 |
| L1PA10_dup3799 | Multi/ | No | — | — | LINE | L1PA\|B\|x | chr9: 115750431-115756749 | − | 38 | 6319 | ZFP37 | Distal | 43912 | EVE annotated | 60 |
| | Uniquely | | | | | | | | | | | | | | |
| Tigger7_dup1730 | Multi | No | — | — | DNA | DNA | chr9: 115760757-115761001 | + | 98 | 245 | ZFP37 | Distal | 39660 | Not documented | 61 |
| Tigger7_dup1731 | Multi/ | No | — | — | DNA | DNA | chr9: 115761020-115761099 | + | 80 | 80 | ZFP37 | Distal | 39562 | Not documented | 62 |
| | Uniquely | | | | | | | | | | | | | | |
| MER21C_dup2959 | Multi/ | No | — | — | LTR | ERVL | chr9: 115761606-115762425 | − | 110 | 820 | ZFP37 | Distal | 38236 | Not documented | 63 |
| | Uniquely | | | | | | | | | | | | | | |
| MER4C_dup631 | Multi/ | No | — | — | LTR | ERV1 | chr9: 117790859-117791209 | + | 57 | 351 | TNC | Proximal | 0 | Not documented | 64 |
| | Uniquely | | | | | | | | | | | | | | |
| MIRc_dup55749 | Uniquely | No | — | — | SINE | MIR | chr9: 117800758-117801006 | + | 130 | 249 | TNC | Proximal | 0 | Not documented | 65 |
| AluSx_dup67838 | Multi | No | — | — | SINE | Alu | chr9: 117801095-117801408 | − | 52 | 314 | TNC | Proximal | 0 | Not documented | 66 |
| MIRb_dup119281 | Multi | No | — | — | SINE | MIR | chr9: 117801466-117801590 | + | 160 | 125 | TNC | Proximal | 0 | Not documented | 67 |
| MIRc_dup55750 | Uniquely | No | — | — | SINE | MIR | chr9: 117801844-117802037 | − | 110 | 194 | TNC | Proximal | 0 | Not documented | 68 |
| MIRb_dup119284 | Multi/ | No | — | — | SINE | MIR | chr9: 117805701-117805907 | + | 120 | 207 | TNC | Proximal | 0 | Not documented | 69 |
| | Uniquely | | | | | | | | | | | | | | |
| MIR3_dup48707 | Multi/ | No | — | — | SINE | MIR | chr9: 117807332-117807472 | + | 140 | 141 | TNC | Proximal | 0 | Not documented | 70 |
| | Uniquely | | | | | | | | | | | | | | |
| AluJb_dup69609 | Multi/ | No | — | — | SINE | Alu | chr9: 117807661-117807960 | − | 76 | 300 | TNC | Proximal | 0 | Not documented | 71 |
| | Uniquely | | | | | | | | | | | | | | |
| MIRb_dup119286 | Multi/ | No | — | — | SINE | MIR | chr9: 117808406-117808613 | − | 120 | 208 | TNC | Proximal | 0 | Not documented | 72 |
| | Uniquely | | | | | | | | | | | | | | |
| MER103C_dup5132 | Multi/ | No | — | — | DNA | DNA | chr9: 117809691-117809966 | − | 100 | 276 | TNC | Proximal | 0 | Not documented | 73 |
| | Uniquely | | | | | | | | | | | | | | |
| MIRb_dup119287 | Multi/ | No | — | — | SINE | MIR | chr9: 117810944-117811152 | − | 120 | 209 | TNC | Proximal | 0 | Not documented | 74 |
| | Uniquely | | | | | | | | | | | | | | |
| MIRc_dup55752 | Multi/ | No | — | — | SINE | MIR | chr9: 117811619-117811824 | − | 140 | 206 | TNC | Proximal | 0 | Not documented | 4675 |
| | Uniquely | | | | | | | | | | | | | | |
| MIRb_dup119288 | Multi | No | — | — | SINE | MIR | chr9: 117812996-117813233 | − | 160 | 238 | TNC | Proximal | 0 | Not documented | 76 |
| MIRc_dup55753 | Multi | No | — | — | SINE | MIR | chr9: 117813609-117813707 | − | 120 | 99 | TNC | Proximal | 0 | Not documented | 77 |
| L1MA7_dup4618 | Multi | No | — | — | LINE | Other L1 | chr9: 117814256-117814309 | + | 34 | 54 | TNC | Proximal | 0 | Not documented | 78 |
| L1MA7_dup4619 | Multi/ | No | — | — | LINE | Other L1 | chr9: 117814377-117814754 | + | 60 | 378 | TNC | Proximal | 0 | Not documented | 79 |
| | Uniquely | | | | | | | | | | | | | | |
| L1MA7_dup4620 | Multi | No | — | — | LINE | Other L1 | chr9: 117814792-117814912 | + | 60 | 121 | TNC | Proximal | 0 | Not documented | 80 |
| MER103C_dup5133 | Multi/ | No | — | — | DNA | DNA | chr9: 117815633-117815923 | − | 140 | 291 | TNC | Proximal | 0 | Not documented | 81 |
| | Uniquely | | | | | | | | | | | | | | |
| MIRb_dup119289 | Multi/ | No | — | — | SINE | MIR | chr9: 117822612-117822852 | + | 150 | 241 | TNC | Proximal | 0 | Not documented | 82 |
| | Uniquely | | | | | | | | | | | | | | |
| Charlie15a_{—} | Multi/ | No | — | — | DNA | DNA | chr9: 117823551-117823745 | + | 150 | 195 | TNC | Proximal | 0 | Not documented | 83 |
| dup1860 | Uniquely | | | | | | | | | | | | | | |
| MIRb_dup119290 | Multi/ | No | — | — | SINE | MIR | chr9: 117824428-117824597 | − | 150 | 170 | TNC | Proximal | 0 | Not documented | 84 |
| | Uniquely | | | | | | | | | | | | | | |
| AluJo_dup34410 | Multi/ | No | — | — | SINE | Alu | chr9: 117824608-117824903 | + | 68 | 296 | TNC | Proximal | 0 | Not documented | 85 |
| | Uniquely | | | | | | | | | | | | | | |
| MER91C_dup857 | Multi | No | — | — | DNA | DNA | chr9: 117826684-117826741 | − | 130 | 58 | TNC | Proximal | 0 | Not documented | 86 |
| AluSx1_dup52598 | Multi/ | No | — | — | SINE | Alu | chr9: 117827393-117827693 | + | 48 | 301 | TNC | Proximal | 0 | Not documented | 87 |
| | Uniquely | | | | | | | | | | | | | | |
| AluSx_dup67839 | Multi/ | No | — | — | SINE | Alu | chr9: 117827936-117828203 | + | 56 | 268 | TNC | Proximal | 0 | Not documented | 88 |
| | Uniquely | | | | | | | | | | | | | | |
| L2a_dup94046 | Uniquely | No | — | — | LINE | L2 | chr9: 117832295-117832799 | − | 150 | 505 | TNC | Proximal | 0 | Not documented | 89 |
| L2c_dup78785 | Uniquely | No | — | — | LINE | L2 | chr9: 117833150-117833450 | + | 160 | 301 | TNC | Proximal | 0 | Not documented | 90 |
| MIRb_dup119293 | Multi/ | No | — | — | SINE | MIR | chr9: 117841918-117842104 | − | 150 | 187 | TNC | Proximal | 0 | Not documented | 91 |
| | Uniquely | | | | | | | | | | | | | | |
| AluSx1_dup52599 | Uniquely | No | — | — | SINE | Alu | chr9: 117842909-117843190 | + | 48 | 282 | TNC | Proximal | 0 | Not documented | 92 |
| AluSx_dup67843 | Multi | No | — | — | SINE | Alu | chr9: 117854849-117855163 | − | 68 | 315 | TNC | Proximal | 0 | Not documented | 93 |
| MIRb_dup119302 | Uniquely | No | — | — | SINE | MIR | chr9: 117859385-117859646 | + | 120 | 262 | TNC | Proximal | 0 | Not documented | 94 |
| L2c_dup78787 | Multi | No | — | — | LINE | L2 | chr9: 117861559-117861659 | − | 110 | 101 | TNC | Proximal | 0 | Not documented | 95 |
| AluYc_dup4289 | Multi/ | No | — | — | SINE | Alu | chr9: 117873158-117873371 | − | 32 | 214 | TNC | Proximal | 0 | Not documented | 96 |
| | Uniquely | | | | | | | | | | | | | | |
| MLT1F2_dup3214 | Multi/ | No | — | — | LTR | ERVL- | chr9: 117873471-117873678 | + | 110 | 208 | TNC | Proximal | 0 | Not documented | 97 |
| | Uniquely | | | | | MaLR | | | | | | | | | |
| AluSq2_dup27071 | Multi | No | — | — | SINE | Alu | chr9: 117873679-117873988 | − | 47 | 310 | TNC | Proximal | 0 | Not documented | 98 |
| MLT1F2_dup3215 | Multi/ | No | — | — | LTR | ERVL- | chr9: 117873989-117874343 | + | 110 | 355 | TNC | Proximal | 0 | Not documented | 99 |
| | Uniquely | | | | | MaLR | | | | | | | | | |
| MIR3_dup48711 | Multi | No | — | — | SINE | MIR | chr9: 117874479-117874586 | + | 140 | 108 | TNC | Proximal | 0 | Not documented | 4700 |
| Charlie15a_{—} | Multi | No | — | — | DNA | DNA | chr9: 117874625-117874769 | − | 130 | 145 | TNC | Proximal | 0 | Not documented | 1 |
| dup1861 | | | | | | | | | | | | | | | |
| LTR52-int_dup99 | Uniquely | No | — | — | LTR | ERVL | chr9: 119468952-119470937 | + | 110 | 1986 | ASTN2 | Proximal | 0 | Not documented | 2 |
| L1PA8_dup4578 | Uniquely | No | — | — | LINE | L1PA\|B\|x | chr9: 119960327-119962362 | − | 43 | 2036 | ASTN2 | Proximal | 0 | Not documented | 3 |
| AluJb_dup69771 | Multi | No | — | — | SINE | Alu | chr9: 123605777-123606070 | + | 54 | 294 | PSMD5 | Proximal | 516 | Not documented | 4 |
| AluJb_dup69904 | Multi | No | — | — | SINE | Alu | chr9: 125769653-125769994 | − | 66 | 342 | RABGAP1 | Proximal | 0 | Not documented | 5 |
| L1MC4a_dup14846 | Multi | No | — | — | LINE | Other L1 | chr9: 125769995-125770104 | − | 130 | 110 | RABGAP1 | Proximal | 0 | Not documented | 6 |
| AluSg7_dup4061 | Multi | No | — | — | SINE | Alu | chr9: 126141082-126141383 | − | 27 | 302 | CRB2 | Proximal | 0 | Not documented | 7 |
| AluSx_dup68221 | Uniquely | No | — | — | SINE | Alu | chr9: 127067552-127067862 | + | 53 | 311 | NEK6 | Proximal | 0 | Not documented | 8 |
| MIRc_dup56583 | Multi | No | — | — | SINE | MIR | chr9: 127114013-127114210 | + | 100 | 198 | NEK6 | Proximal | 0 | Not documented | 9 |
| MER58A_dup7044 | Uniquely | No | — | — | DNA | DNA | chr9: 127119463-127119681 | − | 100 | 219 | PSMB7 | Proximal | 0 | Not documented | 10 |
| AluSc_dup17885 | Multi | No | — | — | SINE | Alu | chr9: 127997487-127997755 | − | 40 | 269 | HSPA5 | Proximal | 0 | Not documented | 11 |
| MIR3_dup49641 | Multi | No | — | — | SINE | MIR | chr9: 130213359-130213440 | − | 140 | 82 | RPL12 | Proximal | 0 | Not documented | 12 |
| MIR3_dup49709 | Multi | No | — | — | SINE | MIR | chr9: 130648286-130648357 | − | 130 | 72 | ST6GALNAC6 | Proximal | 0 | Not documented | 13 |
| L2c_dup79908 | Multi | No | — | — | LINE | L2 | chr9: 130699117-130699217 | − | 140 | 101 | DPM2 | Proximal | 0 | Not documented | 14 |
| MIR_dup95449 | Multi | No | — | — | SINE | MIR | chr9: 130699501-130699659 | + | 150 | 159 | DPM2 | Proximal | 0 | Not documented | 15 |
| MIR3_dup49735 | Multi | No | — | — | SINE | MIR | chr9: 130947680-130947826 | − | 120 | 147 | CIZ1 | Proximal | 0 | Not documented | 16 |
| L1ME4a_dup23997 | Multi | No | — | — | LINE | Other L1 | chr9: 130950127-130950680 | + | 140 | 554 | CIZ1 | Proximal | 0 | Not documented | 17 |
| FAM_dup2649 | Multi | No | — | — | SINE | Alu | chr9: 131071433-131071603 | − | 64 | 171 | TRUB2 | Proximal | 0 | Not documented | 18 |
| MER5B_dup13725 | Multi/ | No | — | — | DNA | DNA | chr9: 131085507-131085577 | − | 150 | 71 | COQ4 | Proximal | 0 | Not documented | 19 |
| | Uniquely | | | | | | | | | | | | | | |
| L2a_dup95181 | Multi | No | — | — | LINE | L2 | chr9: 131085737-131085857 | + | 120 | 121 | COQ4 | Proximal | 0 | Not documented | 20 |
| AluSg_dup20625 | Uniquely | No | — | — | SINE | Alu | chr9: 131140536-131140851 | + | 36 | 316 | URM1 | Proximal | 0 | Not documented | 21 |
| AluSx_dup68762 | Uniquely | No | — | — | SINE | Alu | chr9: 131361419-131361713 | − | 55 | 295 | SPTAN1 | Proximal | 0 | Not documented | 22 |
| L2b_dup52501 | Multi | No | — | — | LINE | L2 | chr9: 131584359-131584564 | − | 140 | 206 | ENDOG | Proximal | 0 | Not documented | 23 |
| MIR_dup95530 | Multi | No | — | — | SINE | MIR | chr9: 131698924-131698994 | + | 75 | 71 | PHYHD1 | Proximal | 0 | Not documented | 24 |
| MER 41C_dup390 | Multi | No | — | — | LTR | ERV1 | chr9: 132100761-132101175 | − | 32 | 415 | IER5L | Distal | 160222 | Not documented | 4725 |
| MIRb_dup122547 | Uniquely | No | — | — | SINE | MIR | chr9: 132397265-132397405 | + | 140 | 141 | NTMT1 | Proximal | 0 | Not documented | 26 |
| AluY_dup61215 | Multi | No | — | — | SINE | Alu | chr9: 132398382-132398689 | + | 30 | 308 | NTMT1 | Proximal | 174 | Not documented | 27 |
| AluSp_dup24407 | Multi/ | No | — | — | SINE | Alu | chr9: 132590057-132590371 | − | 42 | 315 | C9orf78 | Proximal | 0 | Not documented | 28 |
| | Uniquely | | | | | | | | | | | | | | |
| AluSx3_dup14903 | Multi | No | — | — | SINE | Alu | chr9: 133579635-133579934 | + | 49 | 300 | EXOSC2 | Proximal | 0 | Not documented | 29 |
| MIR3_dup49949 | Multi | No | — | — | SINE | MIR | chr9: 133996837-133997002 | − | 130 | 166 | AIF1L | Proximal | 0 | Not documented | 30 |
| L2c_dup80058 | Multi | No | — | — | LINE | L2 | chr9: 133997326-133997463 | − | 150 | 138 | AIF1L | Proximal | 0 | Not documented | 31 |
| MLT1l_dup5874 | Uniquely | No | — | — | LTR | ERVL- | chr9: 13408164-13408508 | + | 140 | 345 | MPDZ | Distal | 128576 | Not documented | 32 |
| | | | | | | MaLR | | | | | | | | | |
| MER112_dup2587 | Multi | No | — | — | DNA | DNA | chr9: 134399864-134400067 | + | 130 | 204 | UCK1 | Proximal | 0 | Not documented | 33 |
| AluJo_dup35007 | Multi/ | No | — | — | SINE | Alu | chr9: 136892566-136892831 | − | 74 | 266 | BRD3 | Distal | 2597 | Not documented | 34 |
| | Uniquely | | | | | | | | | | | | | | |
| LTR79_dup2542 | Multi | No | — | — | LTR | ERVL | chr9: 139687679-139687967 | − | 130 | 289 | RP11-216L13.17 | Proximal | 0 | Not documented | 35 |
| AluSz_dup48348 | Multi/ | No | — | — | SINE | Alu | chr9: 140510539-140510844 | + | 68 | 306 | C9orf37 | Proximal | 0 | Not documented | 36 |
| | Uniquely | | | | | | | | | | | | | | |
| MIR_dup89558 | Uniquely | No | — | — | SINE | MIR | chr9: 14093929-14094171 | − | 130 | 243 | NFIB | Proximal | 0 | Not documented | 37 |
| AluSx_dup64135 | Uniquely | No | — | — | SINE | Alu | chr9: 14233418-14233726 | − | 41 | 309 | NFIB | Proximal | 0 | Not documented | 38 |
| L1MA4A_dup3284 | Uniquely | No | — | — | LINE | Other L1 | chr9: 14637610-14639647 | + | 70 | 2038 | ZDHHC21 | Proximal | 0 | Not documented | 39 |
| L1M5_dup32923 | Multi | No | — | — | LINE | Other L1 | chr9: 15460105-15460232 | − | 120 | 128 | SNAPC3 | Proximal | 0 | Not documented | 40 |
| AluSq_dup9888 | Multi | No | — | — | SINE | Alu | chr9: 15460396-15460687 | − | 47 | 292 | SNAPC3 | Proximal | 0 | Not documented | 41 |
| MER4-int_dup1288 | Multi | No | — | — | LTR | ERV1 | chr9: 21332306-21332442 | + | 110 | 137 | KLHL9 | Proximal | 0 | Not documented | 42 |
| MIRc_dup53082 | Multi | No | — | — | SINE | MIR | chr9: 21967783-21967866 | + | 140 | 84 | CDKN2A | Proximal | 0 | Not documented | 43 |
| AluSx_dup64425 | Multi/ | No | — | — | SINE | Alu | chr9: 22007077-22007388 | + | 50 | 312 | CDKN2B | Proximal | 0 | Not documented | 44 |
| | Uniquely | | | | | | | | | | | | | | |
| L1MC4_dup15120 | Multi | No | — | — | LINE | Other L1 | chr9: 27327317-27327943 | + | 110 | 627 | MOB3B | Proximal | 0 | Not documented | 45 |
| AluJb_dup66476 | Multi | No | — | — | SINE | Alu | chr9: 27327947-27328081 | + | 100 | 135 | MOB3B | Proximal | 0 | Not documented | 46 |
| AluY_dup57054 | Uniquely | No | — | — | SINE | Alu | chr9: 32553198-32553486 | − | 31 | 289 | NDUFB6 | Proximal | 0 | Not documented | 47 |
| LTR33_dup4909 | Uniquely | No | — | — | LTR | ERVL | chr9: 3263687-3264172 | + | 130 | 486 | RFX3 | Proximal | 0 | Not documented | 48 |
| AluSx_dup64617 | Multi | No | — | — | SINE | Alu | chr9: 33045561-33045858 | + | 50 | 298 | SMU1 | Proximal | 0 | Not documented | 49 |
| FLAM_C_dup10318 | Multi | No | — | — | SINE | Alu | chr9: 33461464-33461583 | − | 68 | 120 | NOL6 | Proximal | 0 | Not documented | 4750 |
| L2c_dup75822 | Multi | No | — | — | LINE | L2 | chr9: 33461833-33461916 | + | 120 | 84 | NOL6 | Proximal | 0 | Not documented | 51 |
| Tigger3b_dup3546 | Uniquely | No | — | — | DNA | DNA | chr9: 3434771-3435482 | − | 55 | 712 | RFX3 | Proximal | 0 | Not documented | 52 |
| L2b_dup48558 | Uniquely | No | — | — | LINE | L2 | chr9: 3445735-3446138 | + | 180 | 404 | RFX3 | Proximal | 0 | Not documented | 53 |
| AluJr_dup35571 | Uniquely | No | — | — | SINE | Alu | chr9: 3452309-3452573 | + | 87 | 265 | AL365202.1 | Proximal | 0 | Not documented | 54 |
| AluSc_dup16541 | Multi | No | — | — | SINE | Alu | chr9: 3456867-3457169 | + | 42 | 303 | AL365202.1 | Proximal | 0 | Not documented | 55 |
| L2c_dup75896 | Uniquely | No | — | — | LINE | L2 | chr9: 34637989-34638401 | − | 160 | 413 | GALT | Proximal | 0 | Not documented | 56 |
| L1M4_dup8021 | Multi/ | No | — | — | LINE | Other L1 | chr9: 3477255-3479318 | − | 110 | 2064 | RFX3 | Proximal | 0 | Not documented | 57 |
| | Uniquely | | | | | | | | | | | | | | |
| MLT1G1_dup1890 | Uniquely | No | — | — | LTR | ERVL- | chr9: 3480068-3480518 | − | 110 | 451 | RFX3 | Proximal | 0 | Not documented | 58 |
| | | | | | | MaLR | | | | | | | | | |
| HERVH-int_dup3380 | Uniquely | No | — | — | LTR | ERV1 | chr9: 35020805-35021442 | − | 44 | 638 | C9orf131 | Distal | 19651 | EVE annotated | 59 |
| AluJr4_dup8658 | Uniquely | No | — | — | SINE | Alu | chr9: 3546788-3546897 | + | 45 | 110 | RFX3 | Distal | 20785 | Not documented | 60 |
| L1MEg_dup9586 | Uniquely | No | — | — | LINE | Other L1 | chr9: 3663781-3665108 | + | 140 | 1328 | RFX3 | Distal | 137778 | Not documented | 61 |
| FLAM_A_dup8097 | Multi | No | — | — | SINE | Alu | chr9: 37088296-37088425 | − | 91 | 130 | ZCCHC7 | Distal | 32112 | Not documented | 62 |
| L1MC5_dup10425 | Multi | No | — | — | LINE | Other L1 | chr9: 37089367-37089471 | − | 120 | 105 | ZCCHC7 | Distal | 31066 | Not documented | 63 |
| MER2_dup4964 | Multi | No | — | — | DNA | DNA | chr9: 37089484-37089782 | − | 70 | 299 | ZCCHC7 | Distal | 30755 | Not documented | 64 |
| L1M1_dup4457 | Uniquely | No | — | — | LINE | Other L1 | chr9: 3712730-3719579 | + | 58 | 6850 | GLIS3 | Distal | 104549 | Not documented | 65 |
| AluJo_dup33207 | Multi | No | — | — | SINE | Alu | chr9: 37503295-37503600 | + | 76 | 306 | POLR1E | Proximal | 0 | Not documented | 66 |
| UCON28b_dup45 | Multi/ | No | — | — | Other | Other | chr9: 3975970-3976106 | − | 150 | 137 | GLIS3 | Proximal | 0 | Not documented | 67 |
| | Uniquely | | | | | Repeats | | | | | | | | | |
| UCON28a_dup86 | Multi | No | — | — | Other | Other | chr9: 3976131-3976221 | − | 130 | 91 | GLIS3 | Proximal | 0 | Not documented | 68 |
| | | | | | | Repeats | | | | | | | | | |
| MER45A_dup1711 | Multi | No | — | — | DNA | DNA | chr9: 4057170-4057347 | + | 110 | 178 | GLIS3 | Proximal | 0 | Not documented | 69 |
| L1MCc_dup1940 | Multi | No | — | — | LINE | Other L1 | chr9: 4058462-4058702 | − | 120 | 241 | GLIS3 | Proximal | 0 | Not documented | 70 |
| MIRb_dup115047 | Multi | No | — | — | SINE | MIR | chr9: 41954852-41954939 | + | 110 | 88 | ANKRD20A2 | Distal | 413365 | Not documented | 71 |
| MER90_dup378 | Multi | No | — | — | LTR | ERV1 | chr9: 42018547-42018966 | + | 120 | 420 | ANKRD20A2 | Distal | 349338 | Not documented | 72 |
| MIRb_dup115048 | Multi | No | — | — | SINE | MIR | chr9: 42019102-42019140 | − | 100 | 39 | ANKRD20A2 | Distal | 349164 | Not documented | 73 |
| AluJr4_dup8885 | Multi | No | — | — | SINE | Alu | chr9: 42217142-42217404 | − | 80 | 263 | ANKRD20A2 | Distal | 150900 | Not documented | 74 |
| THE1B-int_dup2471 | Multi | No | — | — | LTR | ERVL- | chr9: 42232823-42234116 | − | 50 | 1294 | ANKRD20A2 | Distal | 134188 | Not documented | 4775 |
| | | | | | | MaLR | | | | | | | | | |
| L1ME1_dup16626 | Multi | No | — | — | LINE | Other L1 | chr9: 42236271-42236717 | + | 130 | 447 | ANKRD20A2 | Distal | 131587 | Not documented | 76 |
| MER58A_dup6695 | Multi | No | — | — | DNA | DNA | chr9: 42236720-42236893 | + | 110 | 174 | ANKRD20A2 | Distal | 131411 | Not documented | 77 |
| AluSc5_dup3341 | Multi | No | — | — | SINE | Alu | chr9: 42236921-42237218 | + | 50 | 298 | ANKRD20A2 | Distal | 131086 | Not documented | 78 |
| L1ME3B_dup4223 | Multi | No | — | — | LINE | Other L1 | chr9: 42247948-42249533 | − | 140 | 1586 | ANKRD20A2 | Distal | 118771 | Not documented | 79 |
| L1PA17_dup2487 | Multi | No | — | — | LINE | L1PA\|B\|x | chr9: 42250144-42250594 | + | 150 | 451 | ANKRD20A2 | Distal | 117710 | Not documented | 80 |
| SST1_dup58 | Multi | No | — | — | Other | Other | chr9: 42251223-42252481 | − | 30 | 1259 | ANKRD20A2 | Distal | 115823 | Not documented | 81 |
| | | | | | | Repeats | | | | | | | | | |
| GSATII_dup13 | Multi | No | — | — | Other | Other | chr9: 42254026-42257767 | + | 45 | 3742 | ANKRD20A2 | Distal | 110537 | Not documented | 82 |
| | | | | | | Repeats | | | | | | | | | |
| Tigger1_dup6598 | Uniquely | No | — | — | DNA | DNA | chr9: 4233342-4234775 | − | 57 | 1434 | GLIS3 | Proximal | 0 | Not documented | 83 |
| MER90a_dup607 | Multi | No | — | — | LTR | ERV1 | chr9: 44246025-44246239 | − | 110 | 215 | BX088651.2 | Distal | 79678 | Not documented | 84 |
| MER90a_dup610 | Multi | No | — | — | LTR | ERV1 | chr9: 44401599-44401814 | + | 110 | 216 | BX088651.1 | Proximal | 0 | Not documented | 85 |
| L1M4_dup8345 | Multi | No | — | — | LINE | Other L1 | chr9: 45352993-45355981 | + | 99 | 2989 | AL354718.1 | Distal | 85293 | Not documented | 86 |
| GSATII_dup14 | Multi | No | — | — | Other | Other | chr9: 45435147-45439527 | − | 49 | 4381 | AL354718.1 | Proximal | 1747 | Not documented | 87 |
| | | | | | | Repeats | | | | | | | | | |
| SST1_dup80 | Multi | No | — | — | Other | Other | chr9: 45441072-45442328 | + | 30 | 1257 | AL354718.1 | Proximal | 0 | Not documented | 88 |
| | | | | | | Repeats | | | | | | | | | |
| L1PA17_dup2494 | Multi | No | — | — | LINE | L1PA\|B\|x | chr9: 45442959-45443409 | − | 150 | 451 | AL354718.1 | Proximal | 1418 | Not documented | 89 |
| L1ME3B_dup4234 | Multi | No | — | — | LINE | Other L1 | chr9: 45444020-45445605 | + | 150 | 1586 | AL354718.1 | Distal | 2479 | Not documented | 90 |
| AluSc5_dup3359 | Multi | No | — | — | SINE | Alu | chr9: 45456305-45456610 | − | 55 | 306 | AL354718.1 | Distal | 14764 | Not documented | 91 |
| MER58A_dup6699 | Multi | No | — | — | DNA | DNA | chr9: 45456630-45456803 | − | 110 | 174 | AL354718.1 | Distal | 15089 | Not documented | 92 |
| AluJr4_dup8888 | Multi | No | — | — | SINE | Alu | chr9: 45476125-45476389 | + | 79 | 265 | AL354718.1 | Distal | 34584 | Not documented | 93 |
| SST1_dup81 | Multi | No | — | — | Other | Other | chr9: 45726673-45727484 | + | 110 | 812 | FAM27A | Proximal | 0 | Not documented | 94 |
| | | | | | | Repeats | | | | | | | | | |
| MIRb_dup115226 | Multi | No | — | — | SINE | MIR | chr9: 46687902-46687940 | + | 100 | 39 | FAM27D1 | Distal | 296347 | Not documented | 95 |
| MER90_dup379 | Multi | No | — | — | LTR | ERV1 | chr9: 46688076-46688301 | − | 110 | 226 | FAM27D1 | Distal | 296521 | Not documented | 96 |
| L1PA13_dup4552 | Multi/ | No | — | — | LINE | L1PA\|B\|x | chr9: 4731115-4731508 | − | 49 | 394 | AK3 | Proximal | 0 | Not documented | 97 |
| | Uniquely | | | | | | | | | | | | | | |
| LTR89_dup579 | Uniquely | No | — | — | LTR | ERVL | chr9: 6011589-6012053 | − | 110 | 465 | RANBP6 | Proximal | 0 | Not documented | 98 |
| L1M1_dup4627 | Multi | No | — | — | LINE | Other L1 | chr9: 66458644-66460514 | − | 76 | 1871 | SPATA31A7 | Distal | 949035 | Not documented | 99 |
| L1PREC2_dup4173 | Multi/ | No | — | — | LINE | L1PA\|B\|x | chr9: 66460512-66462772 | − | 57 | 2261 | SPATA31A7 | Distal | 950903 | Not documented | 4800 |
| | Uniquely | | | | | | | | | | | | | | |
| L1M1_dup4628 | Uniquely | No | — | — | LINE | Other L1 | chr9: 66470890-66473936 | + | 96 | 3047 | SPATA31A7 | Distal | 961281 | Not documented | 1 |
| L1M4_dup8360 | Multi | No | — | — | LINE | Other L1 | chr9: 66490114-66493134 | + | 99 | 3021 | SPATA31A7 | Distal | 980505 | Not documented | 2 |
| MER90_dup382 | Multi | No | — | — | LTR | ERV1 | chr9: 66553056-66553281 | + | 110 | 226 | SPATA31A7 | Distal | 1043447 | Not documented | 3 |
| MIRb_dup115278 | Multi | No | — | — | SINE | MIR | chr9: 66553417-66553455 | − | 100 | 39 | SPATA31A7 | Distal | 1043808 | Not documented | 4 |
| CER_dup15 | Multi/ | No | — | — | Other | Other | chr9: 66858042-66863340 | + | 59 | 5299 | FAM27E3 | Distal | 921605 | Not documented | 5 |
| | Uniquely | | | | | Repeats | | | | | | | | | |
| AluJr4_dup8894 | Multi | No | — | — | SINE | Alu | chr9: 68263861-68264120 | − | 73 | 260 | RP11-195B21.3 | Distal | 275864 | Not documented | 6 |
| MER58A_dup6709 | Multi | No | — | — | DNA | DNA | chr9: 68284830-68285003 | + | 110 | 174 | RP11-195B21.3 | Distal | 296833 | Not documented | 7 |
| SST1_dup106 | Multi | Nc | — | — | Other | Other | chr9: 68299166-68300426 | − | 30 | 1261 | RP11-195B21.3 | Distal | 311169 | Not documented | 8 |
| | | | | | | Repeats | | | | | | | | | |
| L1PA6_dup3369 | Multi | No | — | — | LINE | L1PA\|B\|x | chr9: 68383016-68385185 | + | 46 | 2170 | RP11-195B21.3 | Distal | 395019 | Not documented | 9 |
| L1M1_dup4637 | Multi | No | — | — | LINE | Other L1 | chr9: 68406677-68408546 | + | 76 | 1870 | RP11-195B21.3 | Distal | 418680 | Not documented | 10 |
| L1HS_dup888 | Multi | No | — | — | LINE | L1PA\|B\|x | chr9: 68741410-68741811 | + | 20 | 402 | FOXD4L6 | Distal | 457670 | Not documented | 11 |
| CER_dup21 | Multi | No | — | — | Other | Other | chr9: 68995848-69000000 | + | 65 | 4153 | FOXD4L6 | Distal | 199481 | Not documented | 12 |
| | | | | | | Repeats | | | | | | | | | |
| CER_dup22 | Multi | No | — | — | Other | Other | chr9: 69000026-69004778 | + | 61 | 4753 | FOXD4L6 | Distal | 194703 | Not documented | 13 |
| | | | | | | Repeats | | | | | | | | | |
| L1MA9_dup8910 | Multi | No | — | — | LINE | Other L1 | chr9: 69040126-69042361 | − | 98 | 2236 | FOXD4L6 | Distal | 157120 | Not documented | 14 |
| L1HS_dup890 | Multi | No | — | — | LINE | L1PA\|B\|x | chr9: 69080416-69080817 | + | 20 | 402 | FOXD4L6 | Distal | 118664 | Not documented | 15 |
| Charlie16a_{—} | Multi | No | — | — | DNA | DNA | chr9: 69244997-69245181 | + | 150 | 185 | CBWD6 | Proximal | 0 | Not documented | 16 |
| dup1037 | | | | | | | | | | | | | | | |
| AluJr4_dup8907 | Multi | No | — | — | SINE | Alu | chr9: 70611983-70612247 | − | 79 | 265 | AL591479.1 | Distal | 34343 | Not documented | 17 |
| THE1B-int_dup2511 | Multi | No | — | — | LTR | ERVL- | chr9: 70627650-70628942 | − | 50 | 1293 | AL591479.1 | Distal | 17648 | Not documented | 18 |
| | | | | | | MaLR | | | | | | | | | |
| L1ME1_dup16701 | Multi | No | — | — | LINE | Other L1 | chr9: 70631097-70631543 | + | 130 | 447 | AL591479.1 | Distal | 15047 | Not documented | 19 |
| MER58A_dup6715 | Multi | No | — | — | DNA | DNA | chr9: 70631546-70631719 | + | 110 | 174 | AL591479.1 | Distal | 14871 | Not documented | 20 |
| AluSc5_dup3378 | Multi | No | — | — | SINE | AIL | chr9: 70631747-70632044 | + | 52 | 298 | AL591479.1 | Distal | 14546 | Not documented | 21 |
| L1PA17_dup2506 | Multi | No | — | — | LINE | L1PA\|B\|x | chr9: 70644955-70645405 | + | 150 | 451 | AL591479.1 | Proximal | 1185 | Not documented | 22 |
| SST1_dup107 | Multi | No | — | — | Other | Other | chr9: 70646088-70647346 | − | 29 | 1259 | AL591479.1 | Proximal | 0 | Not documented | 23 |
| | | | | | | Repeats | | | | | | | | | |
| GSATII_dup18 | Multi | No | — | — | Other | Other | chr9: 70648890-70653266 | + | 45 | 4377 | AL591479.1 | Proximal | 0 | Not documented | 24 |
| | | | | | | Repeats | | | | | | | | | |
| Charlie16a_{—} | Multi | No | — | — | DNA | DNA | chr9: 70874245-70874429 | − | 150 | 185 | CBWD3 | Proximal | 0 | Not documented | 4825 |
| dup1039 | | | | | | | | | | | | | | | |
| AluSx3_dup14188 | Uniquely | No | — | — | SINE | Alu | chr9: 71796153-71796463 | − | 47 | 311 | TJP2 | Proximal | 0 | Not documented | 26 |
| Tigger1_dup6880 | Uniquely | No | — | — | DNA | DNA | chr9: 72334547-72336901 | + | 51 | 2355 | PTAR1 | Proximal | 0 | Not documented | 27 |
| Charlie15a_{—} | Uniquely | No | — | — | DNA | DNA | chr9: 72365396-72365472 | − | 89 | 77 | PTAR1 | Proximal | 0 | Not documented | 28 |
| dup1797 | | | | | | | | | | | | | | | |
| AluSz6_dup21895 | Uniquely | No | — | — | SINE | Alu | chr9: 72837773-72838075 | + | 69 | 303 | MAMDC2 | Proximal | 0 | Not documented | 29 |
| L2_dup30068 | Uniquely | No | — | — | LINE | L2 | chr9: 72875983-72876923 | − | 140 | 941 | SMC5 | Proximal | 0 | Not documented | 30 |
| L2_dup31010 | Uniquely | No | — | — | LINE | L2 | chr9: 72908936-72910099 | − | 150 | 1164 | SMC5 | Proximal | 0 | Not documented | 31 |
| Tigger1_dup6881 | Multi/ | No | — | — | DNA | DNA | chr9: 72923840-72924783 | − | 55 | 944 | SMC5 | Proximal | 0 | Not documented | 32 |
| | Uniquely | | | | | | | | | | | | | | |
| Tigger1_dup6882 | Multi/ | No | — | — | DNA | DNA | chr9: 72925064-72926528 | − | 55 | 1465 | SMC5 | Proximal | 0 | Not documented | 33 |
| | Uniquely | | | | | | | | | | | | | | |
| L1ME2_dup6844 | Uniquely | No | — | — | LINE | Other L1 | chr9: 72947464-72948223 | + | 110 | 760 | SMC5 | Proximal | 0 | Not documented | 34 |
| AluSx3_dup14199 | Multi | No | — | — | SINE | Alu | chr9: 72969128-72969440 | − | 37 | 313 | SMC5 | Proximal | 0 | Not documented | 35 |
| AluY_dup58809 | Multi | No | — | — | SINE | Alu | chr9: 74966929-74967247 | + | 44 | 319 | ZFAND5 | Proximal | 0 | Not documented | 36 |
| MIR_dup91594 | Multi | No | — | — | SINE | MIR | chr9: 74967703-74967866 | − | 100 | 164 | ZFAND5 | Proximal | 0 | Not documented | 37 |
| MIR3_dup47136 | Multi/ | No | — | — | SINE | MIR | chr9: 74978356-74978519 | + | 110 | 164 | ZFAND5 | Proximal | 0 | Not documented | 38 |
| | Uniquely | | | | | | | | | | | | | | |
| MIR3_dup47167 | Multi | No | — | — | SINE | MIR | chr9: 75768432-75768542 | − | 140 | 111 | ANXA1 | Proximal | 0 | Not documented | 39 |
| L2c_dup76754 | Multi/ | No | — | — | LINE | L2 | chr9: 75768653-75769047 | − | 160 | 395 | ANXA1 | Proximal | 0 | Not documented | 40 |
| | Uniquely | | | | | | | | | | | | | | |
| AluSz6_dup21941 | Multi | No | — | — | SINE | Alu | chr9: 75769340-75769654 | − | 69 | 315 | ANXA1 | Proximal | 0 | Not documented | 41 |
| MSTB1_dup2590 | Uniquely | No | — | — | LTR | ERVL- | chr9: 75776801-75777170 | − | 87 | 370 | ANXA1 | Proximal | 0 | Not documented | 42 |
| | | | | | | MaLR | | | | | | | | | |
| MSTB1_dup2591 | Multi/ | No | — | — | LTR | ERVL- | chr9: 75777828-75778241 | − | 65 | 414 | ANXA1 | Proximal | 0 | Not documented | 43 |
| | Uniquely | | | | | MaLR | | | | | | | | | |
| L2a_dup91776 | Multi/ | No | — | — | LINE | L2 | chr9: 75779107-75779216 | + | 150 | 110 | ANXA1 | Proximal | 0 | Not documented | 44 |
| | Uniquely | | | | | | | | | | | | | | |
| MIRc_dup54003 | Multi | No | — | — | SINE | MIR | chr9: 75779505-75779635 | − | 170 | 131 | ANXA1 | Proximal | 0 | Not documented | 45 |
| L2b_dup49949 | Multi | No | — | — | LINE | L2 | chr9: 75779787-75779901 | − | 170 | 115 | ANXA1 | Proximal | 0 | Not documented | 46 |
| L4_dup9645 | Multi/ | No | — | — | LINE | RTE | chr9: 75780552-75780700 | − | 140 | 149 | ANXA1 | Proximal | 0 | Not documented | 47 |
| | Uniquely | | | | | | | | | | | | | | |
| L1PA16_dup7682 | Multi | No | — | — | LINE | L1PA\|B\|x | chr9: 75781881-75782041 | − | 75 | 161 | ANXA1 | Proximal | 0 | Not documented | 48 |
| L2c_dup76757 | Multi/ | No | — | — | LINE | L2 | chr9: 75783266-75783541 | − | 170 | 276 | ANXA1 | Proximal | 0 | Not documented | 49 |
| | Uniquely | | | | | | | | | | | | | | |
| L2b_dup50025 | Multi | No | — | — | LINE | L2 | chr9: 78609638-78610285 | + | 160 | 648 | PCSK5 | Proximal | 0 | Not documented | 4850 |
| FLAM_C_dup10538 | Multi | No | — | — | SINE | Alu | chr9: 79001786-79001917 | − | 52 | 132 | RFK | Proximal | 0 | Not documented | 51 |
| LTR6A_dup99 | Multi | No | — | — | LTR | ERV1 | chr9: 79013621-79013650 | − | 15 | 30 | RFK | Distal | 4189 | Not documented | 52 |
| MER58B_dup3500 | Multi | No | — | — | DNA | DNA | chr9: 79228190-79228476 | − | 77 | 287 | PRUNE2 | Proximal | 0 | Not documented | 53 |
| MIRb_dup116111 | Multi | No | — | — | SINE | MIR | chr9: 79242610-79242848 | + | 170 | 239 | PRUNE2 | Proximal | 0 | Not documented | 54 |
| L1M5_dup34026 | Uniquely | No | — | — | LINE | Other L1 | chr9: 84276774-84277023 | + | 130 | 250 | TLE1 | Proximal | 0 | Not documented | 55 |
| MER52D_dup338 | Multi/ | No | — | — | LTR | ERV1 | chr9: 84304228-84304442 | − | 95 | 215 | TLE1 | Proximal | 9 | Not documented | 56 |
| | Uniquely | | | | | | | | | | | | | | |
| HSMAR1_dup407 | Uniquely | No | — | — | DNA | DNA | chr9: 86327249-86328520 | + | 36 | 1272 | UBQLN1 | Distal | 4132 | Not documented | 57 |
| SVA_D_dup691 | Multi | No | — | — | Other | SVA | chr9: 86343658-86344307 | + | 6.8 | 650 | GKAP1 | Distal | 10030 | Not documented | 58 |
| AluSx_dup66335 | Uniquely | No | — | — | SINE | Alu | chr9: 87357705-87358015 | − | 47 | 311 | NTRK2 | Proximal | 0 | Not documented | 59 |
| L1MA1_dup2109 | Uniquely | No | — | — | LINE | Other L1 | chr9: 87379520-87380270 | + | 40 | 751 | NTRK2 | Proximal | 0 | Not documented | 60 |
| Charlie8_dup1482 | Multi/ | No | — | — | DNA | DNA | chr9: 87428092-87428351 | − | 110 | 260 | NTRK2 | Proximal | 0 | Not documented | 61 |
| | Uniquely | | | | | | | | | | | | | | |
| L3b_dup3911 | Multi/ | No | — | — | LINE | CR1 | chr9: 87429554-87429657 | − | 150 | 104 | NTRK2 | Proximal | 0 | Not documented | 62 |
| | Uniquely | | | | | | | | | | | | | | |
| L2a_dup92386 | Uniquely | No | — | — | LINE | L2 | chr9: 87455275-87456415 | + | 130 | 1141 | NTRK2 | Proximal | 0 | Not documented | 63 |
| MIR3_dup47524 | Multi/ | No | — | — | SINE | MIR | chr9: 88641429-88641513 | + | 93 | 85 | GOLM1 | Proximal | 0 | Not documented | 64 |
| | Uniquely | | | | | | | | | | | | | | |
| L1PA7_dup7471 | Uniquely | No | — | — | LINE | L1PA\|B\|x | chr9: 88703373-88706134 | − | 38 | 2762 | GOLM1 | Proximal | 0 | Not documented | 65 |
| MIR3_dup47528 | Multi | No | — | — | SINE | MIR | chr9: 88880173-88880299 | + | 160 | 127 | ISCA1 | Proximal | 0 | Not documented | 66 |
| FLAM_A_dup8304 | Multi | No | — | — | SINE | Alu | chr9: 91925237-91925370 | − | 61 | 134 | CKS2 | Proximal | 744 | Not documented | 67 |
| MLT1C_dup10082 | Multi | No | — | — | LTR | ERVL- | chr9: 94053202-94053561 | − | 96 | 360 | AUH | Proximal | 0 | Not documented | 68 |
| | | | | | | MaLR | | | | | | | | | |
| L1MC3_dup6953 | Uniquely | No | — | — | LINE | Other L1 | chr9: 94920164-94921242 | − | 100 | 1079 | SPTLC1 | Distal | 42499 | Not documented | 69 |
| MARNA_dup1906 | Multi | No | — | — | DNA | DNA | chr9: 96208877-96209108 | − | 150 | 232 | FAM120AOS | Proximal | 0 | Not documented | 70 |
| MIR3_dup47728 | Uniquely | No | — | — | SINE | MIR | chr9: 99179153-99179305 | + | 130 | 153 | ZNF367 | Proximal | 0 | Not documented | 71 |
| L1M4c_dup3063 | Multi | No | — | — | LINE | Other L1 | chr9: 99572259-99572953 | + | 96 | 695 | ZNF782 | Distal | 5802 | Not documented | 72 |
| AluJo_dup33970 | Multi/ | No | — | — | SINE | Alu | chr9: 99572954-99573261 | + | 68 | 308 | ZNF782 | Distal | 5494 | Not documented | 73 |
| | Uniquely | | | | | | | | | | | | | | |
| L1ME4a_dup25432 | Multi | No | — | — | LINE | Other L1 | chrX: 100882046-100882479 | + | 130 | 434 | ARMCX3 | Proximal | 0 | Not documented | 74 |
| Mam_R4_dup354 | Multi | No | — | — | LINE | Dong-R4 | chrX: 100910404-100910637 | + | 150 | 234 | ARMCX2 | Proximal | 0 | Not documented | 4875 |
| L2c_dup84065 | Uniquely | No | — | — | LINE | L2 | chrX: 101145882-101146713 | − | 160 | 832 | ZMAT1 | Proximal | 0 | Not documented | 76 |
| L2c_dup84067 | Uniquely | No | — | — | LINE | L2 | chrX: 101148543-101149114 | − | 150 | 572 | ZMAT1 | Proximal | 0 | Not documented | 77 |
| L1ME4a_dup25455 | Multi | No | — | — | LINE | Other L1 | chrX: 102317439-102317695 | − | 120 | 257 | BEX1 | Proximal | 0 | Not documented | 78 |
| L1ME4a_dup25459 | Multi | No | — | — | LINE | Other L1 | chrX: 102508104-102508350 | − | 160 | 247 | TCEAL8 | Proximal | 0 | Not documented | 79 |
| L1MEe_dup7337 | Multi | No | — | — | LINE | Other L1 | chrX: 102508348-102508595 | + | 140 | 248 | TCEAL8 | Proximal | 0 | Not documented | 80 |
| HAL1b_dup2911 | Multi | No | — | — | LINE | Other L1 | chrX: 102586313-102586585 | + | 130 | 273 | TCEAL7 | Proximal | 0 | Not documented | 81 |
| L1MEe_dup7338 | Multi | No | — | — | LINE | Other L1 | chrX: 102586614-102586778 | − | 130 | 165 | TCEAL7 | Proximal | 0 | Not documented | 82 |
| L1ME4a_dup25464 | Multi | No | — | — | LINE | Other L1 | chrX: 102586831-102587051 | + | 120 | 221 | TCEAL7 | Proximal | 0 | Not documented | 83 |
| L1ME4a_dup25469 | Multi | No | — | — | LINE | Other L1 | chrX: 102612902-102613135 | − | 120 | 234 | WBP5 | Proximal | 0 | Not documented | 84 |
| L1ME4a_dup25470 | Multi | No | — | — | LINE | Other L1 | chrX: 102613211-102613374 | + | 110 | 164 | WBP5 | Proximal | 0 | Not documented | 85 |
| L1ME4a_dup25471 | Multi | No | — | — | LINE | Other L1 | chrX: 102632961-102633117 | + | 150 | 157 | NGFRAP1 | Proximal | 0 | Not documented | 86 |
| L1ME4a_dup25477 | Multi | No | — | — | LINE | Other L1 | chrX: 102842387-102842565 | + | 120 | 179 | TCEAL4 | Proximal | 0 | Not documented | 87 |
| L1MEe_dup7339 | Multi | No | — | — | LINE | Other L1 | chrX: 102885306-102885487 | − | 130 | 182 | TCEAL1 | Proximal | 0 | Not documented | 88 |
| L1ME4a_dup25479 | Multi | No | — | — | LINE | Other L1 | chrX: 102885544-102885769 | + | 110 | 226 | TCEAL1 | Proximal | 0 | Not documented | 89 |
| MIRc_dup59714 | Multi | No | — | — | SINE | MIR | chrX: 102939993-102940152 | − | 150 | 160 | MORF4L2 | Proximal | 0 | Not documented | 90 |
| L1M2_dup5366 | Multi | No | — | — | LINE | Other L1 | chrX: 108296198-108297349 | − | 90 | 1152 | IRS4 | Distal | 316548 | Not documented | 91 |
| L1MA1_dup2762 | Multi | No | — | — | LINE | Other L1 | chrX: 115047427-115052815 | − | 66 | 5389 | RP1-241P17.4 | Distal | 93759 | Not documented | 92 |
| L1PB1_dup8746 | Uniquely | No | — | — | LINE | L1PA\|B\|x | chrX: 128340455-128345249 | − | 50 | 4795 | SMARCA1 | Distal | 235232 | EVE annotated | 93 |
| L1M1_dup6144 | Uniquely | No | — | — | LINE | Other L1 | chrX: 128404800-128408063 | − | 77 | 3264 | SMARCA1 | Distal | 172418 | Not documented | 94 |
| L2_dup34517 | Uniquely | No | — | — | LINE | L2 | chrX: 128636099-128637656 | − | 160 | 1558 | SMARCA1 | Proximal | 0 | Not documented | 95 |
| L2_dup34518 | Uniquely | No | — | — | LINE | L2 | chrX: 128638247-128638524 | − | 160 | 278 | SMARCA1 | Proximal | 0 | Not documented | 96 |
| L1MB8_dup9683 | Multi | No | — | — | LINE | Other L1 | chrX: 135045177-135045477 | + | 63 | 301 | MMGT1 | Proximal | 0 | Not documented | 97 |
| AluJr_dup41082 | Multi | No | — | — | SINE | Alu | chrX: 135748519-135748679 | − | 70 | 161 | ARHGEF6 | Proximal | 0 | Not documented | 98 |
| MIRb_dup123887 | Multi | No | — | — | SINE | MIR | chrX: 13721953-13722083 | − | 130 | 131 | RAB9A | Proximal | 0 | Not documented | 99 |
| AluSp_dup24808 | Multi | No | — | — | SINE | Alu | chrX: 13731081-13731238 | + | 60 | 158 | TRAPPC2 | Proximal | 0 | Not documented | 4900 |
| AluSx_dup70506 | Multi | No | — | — | SINE | Alu | chrX: 13731287-13731596 | + | 53 | 310 | TRAPPC2 | Proximal | 0 | Not documented | 1 |
| AluSg_dup21073 | Multi | No | — | — | SINE | Alu | chrX: 13731655-13731944 | + | 42 | 290 | TRAPPC2 | Proximal | 0 | Not documented | 2 |
| AluSg_dup21074 | Multi | No | — | — | SINE | Alu | chrX: 13731983-13732282 | + | 33 | 300 | TRAPPC2 | Proximal | 0 | Not documented | 3 |
| MER45B_dup837 | Multi | No | — | — | DNA | DNA | chrX: 13848046-13849016 | + | 89 | 971 | GPM6B | Proximal | 0 | Not documented | 4 |
| AluJb_dup72128 | Multi/ | No | — | — | SINE | Alu | chrX: 13895662-13895970 | + | 72 | 309 | GPM6B | Proximal | 0 | Not documented | 5 |
| | Uniquely | | | | | | | | | | | | | | |
| MER5A_dup21194 | Multi | No | — | — | DNA | DNA | chrX: 148562320-148562378 | + | 92 | 59 | IDS | Proximal | 0 | Not documented | 6 |
| MER33_dup5537 | Multi | No | — | — | DNA | DNA | chrX: 148563767-148563828 | + | 66 | 62 | IDS | Proximal | 0 | Not documented | 7 |
| L1ME4a_dup26319 | Multi | No | — | — | LINE | Other L1 | chrX: 150574560-150574681 | − | 110 | 122 | VMA21 | Proximal | 0 | Not documented | 8 |
| AluSx1_dup58256 | Multi | No | — | — | SINE | Alu | chrX: 150574837-150575152 | − | 47 | 316 | VMA21 | Proximal | 0 | Not documented | 9 |
| AluSq_dup11637 | Multi/ | No | — | — | SINE | Alu | chrX: 150576351-150576663 | − | 46 | 313 | VMA21 | Proximal | 0 | Not documented | 10 |
| | Uniquely | | | | | | | | | | | | | | |
| MamRep434_dup1432 | Multi | No | — | — | DNA | DNA | chrX: 150577152-150577410 | + | 120 | 259 | VMA21 | Proximal | 0 | Not documented | 11 |
| MIRc_dup61446 | Multi | No | — | — | SINE | MIR | chrX: 150577440-150577530 | − | 120 | 91 | VMA21 | Proximal | 0 | Not documented | 12 |
| L1PA5_dup7385 | Multi | No | — | — | LINE | L1PA\|B\|x | chrX: 152713367-152714006 | + | 19 | 640 | TREX2 | Proximal | 0 | Not documented | 13 |
| L1M2_dup5625 | Multi | No | — | — | LINE | Other L1 | chrX: 153213909-153214098 | + | 130 | 190 | HCFC1 | Proximal | 0 | Not documented | 14 |
| MIR3_dup54468 | Multi/ | No | — | — | SINE | MIR | chrX: 153712995-153713070 | + | 120 | 76 | UBL4A | Proximal | 0 | Not documented | 15 |
| | Uniquely | | | | | | | | | | | | | | |
| L4_dup10206 | Multi | No | — | — | LINE | RTE | chrX: 15870568-15870629 | − | 97 | 62 | AP1S2 | Proximal | 0 | Not documented | 16 |
| MIR_dup96917 | Multi | No | — | — | SINE | MIR | chrX: 20143100-20143312 | − | 140 | 213 | EIF1AX | Proximal | 0 | Not documented | 17 |
| L4_dup10225 | Multi | No | — | — | LINE | RTE | chrX: 20146931-20147208 | + | 160 | 278 | EIF1AX | Proximal | 0 | Not documented | 18 |
| FLAM_A_dup8718 | Multi | No | — | — | SINE | Alu | chrX: 20147408-20147530 | − | 96 | 123 | EIF1AX | Proximal | 0 | Not documented | 19 |
| AluSx3_dup15183 | Multi | No | — | — | SINE | Alu | chrX: 20148138-20148449 | + | 36 | 312 | EIF1AX | Proximal | 0 | Not documented | 20 |
| L1PA15_dup4611 | Multi | No | — | — | LINE | L1PA\|B\|x | chrX: 23874497-23874671 | + | 55 | 175 | APOO | Proximal | 0 | Not documented | 21 |
| AluSx_dup70094 | Multi | No | — | — | SINE | Alu | chrX: 2405410-2405670 | − | 49 | 261 | ZBED1 | Proximal | 0 | Not documented | 22 |
| AluSq2_dup27830 | Multi | No | — | — | SINE | Alu | chrX: 2406227-2406515 | − | 45 | 289 | ZBED1 | Proximal | 0 | Not documented | 23 |
| MSTA_dup11165 | Uniquely | No | — | — | LTR | ERVL- | chrX: 3195564-3195951 | − | 60 | 388 | CXorf28 | Proximal | 0 | Not documented | 24 |
| | | | | | | MaLR | | | | | | | | | |
| MIR_dup96109 | Uniquely | No | — | — | SINE | MIR | chrX: 3202372-3202458 | + | 110 | 87 | CXorf28 | Proximal | 0 | Not documented | 4925 |
| L2c_dup81684 | Multi | No | — | — | LINE | L2 | chrX: 37699073-37699122 | + | 85 | 50 | DYNLT3 | Proximal | 0 | Not documented | 26 |
| L2a_dup97427 | Multi | No | — | — | LINE | L2 | chrX: 38185409-38185542 | + | 140 | 134 | RPGR | Proximal | 0 | Not documented | 27 |
| MER112_dup2674 | Multi | No | — | — | DNA | DNA | chrX: 38185612-38185851 | + | 110 | 240 | RPGR | Proximal | 0 | Not documented | 28 |
| L1M1_dup5005 | Uniquely | No | — | — | LINE | Other L1 | chrX: 40579359-40582635 | + | 61 | 3277 | MED14 | Proximal | 0 | Not documented | 29 |
| MER34B-int_dup448 | Multi | No | — | — | LTR | ERV1 | chrX: 41534155-41534332 | − | 57 | 178 | CASK | Proximal | 0 | Not documented | 30 |
| MIR3_dup51195 | Multi/ | No | — | — | SINE | MIR | chrX: 43626155-43626309 | + | 120 | 155 | MAOB | Proximal | 0 | Not documented | 31 |
| | Uniquely | | | | | | | | | | | | | | |
| AluSz_dup49950 | Multi | No | — | — | SINE | Alu | chrX: 46333091-46333386 | − | 49 | 296 | KRBOX4 | Proximal | 0 | Not documented | 32 |
| MER44C_dup555 | Multi | No | — | — | DNA | DNA | chrX: 46333387-46333921 | + | 75 | 535 | KRBOX4 | Proximal | 0 | Not documented | 33 |
| AluSx_dup69708 | Multi | No | — | — | SINE | Alu | chrX: 47054634-47054949 | − | 39 | 316 | UBA1 | Proximal | 0 | Not documented | 34 |
| L1MEe_dup7131 | Multi | No | — | — | LINE | Other L1 | chrX: 48381959-48382117 | − | 110 | 159 | EBP | Proximal | 0 | Not documented | 35 |
| L2b_dup53998 | Multi | No | — | — | LINE | L2 | chrX: 48932114-48932206 | + | 140 | 93 | AF196779.12 | Proximal | 0 | Not documented | 36 |
| L1ME3B_dup4632 | Multi | No | — | — | LINE | Other L1 | chrX: 48963785-48964056 | + | 130 | 272 | WDR45 | Distal | 5678 | Not documented | 37 |
| MIR_dup98343 | Multi | No | — | — | SINE | MIR | chrX: 48964086-48964279 | + | 120 | 194 | WDR45 | Distal | 5979 | Not documented | 38 |
| MER117_dup2554 | Multi/ | No | — | — | DNA | DNA | chrX: 51645221-51645375 | − | 150 | 155 | MAGED1 | Proximal | 0 | Not documented | 39 |
| | Uniquely | | | | | | | | | | | | | | |
| MIR_dup98590 | Multi | No | — | — | SINE | MIR | chrX: 53406072-53406224 | − | 78 | 153 | SMC1A | Proximal | 0 | Not documented | 40 |
| L2c_dup82363 | Uniquely | No | — | — | LINE | L2 | chrX: 53406222-53406477 | + | 120 | 256 | SMC1A | Proximal | 0 | Not documented | 41 |
| L1PA10_dup3985 | Uniquely | No | — | — | LINE | L1PA\|B\|x | chrX: 53996792-53998475 | + | 35 | 1684 | PHF8 | Proximal | 0 | Not documented | 42 |
| MER117_dup2556 | Multi/ | No | — | — | DNA | DNA | chrX: 54842311-54842447 | − | 120 | 137 | MAGED2 | Proximal | 0 | Not documented | 43 |
| | Uniquely | | | | | | | | | | | | | | |
| L3_dup26524 | Multi | No | — | — | LINE | CR1 | chrX: 54953294-54953419 | − | 140 | 126 | TRO | Proximal | 0 | Not documented | 44 |
| L1PA15_dup4775 | Uniquely | No | — | — | LINE | L1PA\|B\|x | chrX: 56020642-56023332 | − | 95 | 2691 | AL353698.1 | Distal | 77426 | Not documented | 45 |
| MamRep605_dup2815 | Multi | No | — | — | Other | Other | chrX: 56843587-56843944 | + | 160 | 358 | SPIN3 | Distal | 158860 | Not documented | 46 |
| | | | | | | Repeats | | | | | | | | | |
| HERVH48-int_dup68 | Multi | No | — | — | LTR | ERV1 | chrX: 57245761-57246886 | + | 28 | 1126 | FAAH2 | Distal | 66254 | Not documented | 47 |
| L1PA4_dup7088 | Uniquely | No | — | — | LINE | L1PA\|B\|x | chrX: 57650003-57655813 | + | 30 | 5811 | ZXDB | Distal | 26098 | EVE annotated | 48 |
| AluSz_dup48703 | Multi | No | — | — | SINE | Alu | chrX: 5906824-5907138 | + | 52 | 315 | NLGN4X | Proximal | 0 | Not documented | 49 |
| MER57-int_dup693 | Uniquely | No | — | — | LTR | ERV1 | chrX: 62619420-62619784 | − | 66 | 365 | SPIN4 | Distal | 48198 | Not documented | 4950 |
| LTR46-int_dup35 | Multi | No | — | — | LTR | ERV1 | chrX: 62646749-62647932 | − | 130 | 1184 | SPIN4 | Distal | 75527 | EVE annotated | 51 |
| LTR46-int_dup34 | Multi/ | No | — | — | LTR | ERV1 | chrX: 62648256-62653802 | − | 94 | 5547 | SPIN4 | Distal | 77034 | EVE annotated | 52 |
| | Uniquely | | | | | | | | | | | | | | |
| MIRb_dup127175 | Multi | No | — | — | SINE | MIR | chrX: 69509377-69509497 | − | 170 | 121 | PDZD11 | Proximal | 0 | Not documented | 53 |
| AluSg7_dup4346 | Multi | No | — | — | SINE | Alu | chrX: 70520285-70520369 | − | 37 | 85 | NONO | Proximal | 0 | Not documented | 54 |
| AluSg7_dup4347 | Multi | No | — | — | SINE | Alu | chrX: 70520370-70520464 | − | 48 | 95 | NONO | Proximal | 0 | Not documented | 55 |
| L2a_dup98877 | Multi | No | — | — | LINE | L2 | chrX: 73042269-73042349 | + | 130 | 81 | CHIC1 | Distal | 135333 | Not documented | 56 |
| L2a_dup98878 | Multi | No | — | — | LINE | L2 | chrX: 73043321-73043383 | + | 150 | 63 | CHIC1 | Distal | 136385 | Not documented | 57 |
| MIRc_dup59147 | Multi | No | — | — | SINE | MIR | chrX: 73043930-73043999 | − | 140 | 70 | CHIC1 | Distal | 136994 | Not documented | 58 |
| L2b_dup54578 | Multi | No | — | — | LINE | L2 | chrX: 73044011-73044061 | + | 140 | 51 | CHIC1 | Distal | 137075 | Not documented | 59 |
| MIRc_dup59148 | Multi | No | — | — | SINE | MIR | chrX: 73044222-73044279 | − | 140 | 58 | CHIC1 | Distal | 137286 | Not documented | 60 |
| L2b_dup54579 | Multi | No | — | — | LINE | L2 | chrX: 73044308-73044351 | + | 110 | 44 | CHIC1 | Distal | 137372 | Not documented | 61 |
| MIRc_dup59149 | Multi | No | — | — | SINE | MIR | chrX: 73044838-73044890 | − | 87 | 53 | CHIC1 | Distal | 137902 | Not documented | 62 |
| L2b_dup54580 | Multi | No | — | — | LINE | L2 | chrX: 73044936-73044977 | + | 140 | 42 | CHIC1 | Distal | 138000 | Not documented | 63 |
| MIRc_dup59150 | Multi | No | — | — | SINE | MIR | chrX: 73045246-73045287 | − | 87 | 42 | CHIC1 | Distal | 138310 | Not documented | 64 |
| L2b_dup54581 | Multi | No | — | — | LINE | L2 | chrX: 73045307-73045352 | + | 160 | 46 | CHIC1 | Distal | 138371 | Not documented | 65 |
| MER58C_dup1513 | Multi | No | — | — | DNA | DNA | chrX: 73045399-73045570 | + | 110 | 172 | CHIC1 | Distal | 138463 | Not documented | 66 |
| L1MC3_dup7498 | Multi/ | No | — | — | LINE | Other L1 | chrX: 73056273-73057558 | + | 68 | 1286 | CHIC1 | Distal | 149337 | Not documented | 67 |
| | Uniquely | | | | | | | | | | | | | | |
| MIRc_dup59151 | Multi | No | — | — | SINE | MIR | chrX: 73061652-73061776 | − | 150 | 125 | CHIC1 | Distal | 154716 | Not documented | 68 |
| FLAM_C_dup11549 | Multi | No | — | — | SINE | Alu | chrX: 73067240-73067354 | − | 51 | 115 | CHIC1 | Distal | 160304 | Not documented | 69 |
| AluSp_dup25694 | Multi | No | — | — | SINE | Alu | chrX: 73166776-73167088 | − | 39 | 313 | CHIC1 | Distal | 259840 | Not documented | 70 |
| L1M4b_dup3612 | Uniquely | No | — | — | LINE | Other L1 | chrX: 73185368-73186025 | − | 110 | 658 | CHIC1 | Distal | 278432 | Not documented | 71 |
| L1MD3_dup2928 | Multi | No | — | — | LINE | Other L1 | chrX: 73193671-73194597 | − | 120 | 927 | CHIC1 | Distal | 286735 | Not documented | 72 |
| AluSp_dup25698 | Multi | No | — | — | SINE | Alu | chrX: 73214662-73214873 | − | 41 | 212 | CHIC1 | Distal | 307726 | Not documented | 73 |
| L1MA7_dup5030 | Multi | No | — | — | LINE | Other L1 | chrX: 73218467-73218733 | + | 120 | 267 | ZCCHC13 | Distal | 305293 | Not documented | 74 |
| L1MA7_dup5031 | Multi | No | — | — | LINE | Other L1 | chrX: 73219042-73220370 | + | 120 | 1329 | ZCCHC13 | Distal | 303656 | Not documented | 4975 |
| L1MA7_dup5035 | Multi | No | — | — | LINE | Other L1 | chrX: 73223421-73224585 | + | 98 | 1165 | ZCCHC13 | Distal | 299441 | Not documented | 76 |
| L1PA8_dup4842 | Multi | No | — | — | LINE | L1PA\|B\|x | chrX: 73291825-73297937 | − | 48 | 6113 | ZCCHC13 | Distal | 226089 | Not documented | 77 |
| L1MA9_dup9870 | Uniquely | No | — | — | LINE | Other L1 | chrX: 73332664-73334841 | + | 80 | 2178 | ZCCHC13 | Distal | 189185 | Not documented | 78 |
| HERVH-int_dup3693 | Multi | No | — | — | LTR | ERV1 | chrX: 73341830-73346583 | − | 82 | 4754 | ZCCHC13 | Distal | 177443 | EVE annotated | 79 |
| HERVH-int_dup3695 | Uniquely | No | — | — | LTR | ERV1 | chrX: 73348549-73350032 | − | 110 | 1484 | ZCCHC13 | Distal | 173994 | Not documented | 80 |
| L1MA9_dup9871 | Multi | No | — | — | LINE | Other L1 | chrX: 73416212-73418390 | − | 77 | 2179 | ZCCHC13 | Distal | 105636 | Not documented | 81 |
| HSMAR2_dup1053 | Uniquely | No | — | — | DNA | DNA | chrX: 73418491-73419776 | + | 52 | 1286 | ZCCHC13 | Distal | 104250 | Not documented | 82 |
| L1MB7_dup12423 | Multi | No | — | — | LINE | Other L1 | chrX: 73445598-73446502 | + | 120 | 905 | ZCCHC13 | Distal | 77524 | Not documented | 83 |
| AluJb_dup74739 | Multi | No | — | — | SINE | Alu | chrX: 73446519-73446713 | + | 65 | 195 | ZCCHC13 | Distal | 77313 | Not documented | 84 |
| L1MB7_dup12424 | Multi | No | — | — | LINE | Other L1 | chrX: 73446717-73447024 | + | 93 | 308 | ZCCHC13 | Distal | 77002 | Not documented | 85 |
| AluJo_dup36947 | Multi | No | — | — | SINE | Alu | chrX: 73447025-73447328 | + | 78 | 304 | ZCCHC13 | Distal | 76698 | Not documented | 86 |
| MER21C_dup3095 | Multi | No | — | — | LTR | ERVL | chrX: 73471284-73471584 | + | 99 | 301 | ZCCHC13 | Distal | 52442 | Not documented | 87 |
| MER21C_dup3096 | Multi | No | — | — | LTR | ERVL | chrX: 73471652-73471724 | + | 74 | 73 | ZCCHC13 | Distal | 52302 | Not documented | 88 |
| AluY_dup64006 | Multi/ | No | — | — | SINE | Alu | chrX: 73472111-73472412 | + | 33 | 302 | ZCCHC13 | Distal | 51614 | Not documented | 89 |
| | Uniquely | | | | | | | | | | | | | | |
| AluJb_dup74773 | Uniquely | No | — | — | SINE | Alu | chrX: 73806647-73806949 | + | 70 | 303 | RLIM | Proximal | 0 | Not documented | 90 |
| L1PA4_dup7277 | Uniquely | No | — | — | LINE | L1PA\|B\|x | chrX: 74533357-74539492 | − | 27 | 6136 | UPRT | Distal | 8923 | EVE annotated | 91 |
| HERVK9-int_dup389 | Uniquely | No | — | — | LTR | ERVK | chrX: 74547838-74549194 | + | 28 | 1357 | UPRT | Distal | 23404 | Not documented | 92 |
| L1P4a_dup349 | Uniquely | No | — | — | LINE | L1PA\|B\|x | chrX: 74892845-74894486 | + | 110 | 1642 | MAGEE2 | Distal | 108338 | Not documented | 93 |
| L1PA7_dup8083 | Uniquely | No | — | — | LINE | L1PA\|B\|x | chrX: 74939868-74944836 | − | 55 | 4969 | MAGEE2 | Distal | 57988 | Not documented | 94 |
| AluSp_dup25733 | Uniquely | No | — | — | SINE | Alu | chrX: 74960039-74960331 | − | 34 | 293 | MAGEE2 | Distal | 42493 | Not documented | 95 |
| MIRc_dup59239 | Multi | No | — | — | SINE | MIR | chrX: 77385280-77385483 | − | 100 | 204 | TAF9B | Proximal | 0 | Not documented | 96 |
| Plat_L3_dup2179 | Multi | No | — | — | LINE | CR1 | chrX: 77386008-77386068 | + | 100 | 61 | TAF9B | Proximal | 0 | Not documented | 97 |
| L1PA10_dup4158 | Uniquely | No | — | — | LINE | L1PA\|B\|x | chrX: 82690745-82694929 | − | 54 | 4185 | POU3F4 | Distal | 68341 | Not documented | 98 |
| L1M1_dup5628 | Uniquely | No | — | — | LINE | Other L1 | chrX: 86816114-86819435 | + | 90 | 3322 | KLHL4 | Proximal | 0 | Not documented | 99 |
| L1PB1_dup8302 | Uniquely | No | — | — | LINE | L1PA\|B\|x | chrX: 86892966-86896024 | + | 50 | 3059 | KLHL4 | Proximal | 0 | Not documented | 5000 |
| L1PB1_dup8303 | Uniquely | No | — | — | LINE | L1PA\|B\|x | chrX: 86896035-86898076 | + | 53 | 2042 | KLHL4 | Proximal | 0 | Not documented | 1 |
| LTR6A_dup115 | Multi | No | — | — | LTR | ERV1 | chrX: 86959195-86959224 | + | 15 | 30 | KLHL4 | Distal | 34146 | Not documented | 2 |
| LTR4_dup36 | Multi | No | — | — | LTR | ERV1 | chrX: 89713046-89713602 | − | 74 | 557 | TGIF2LX | Distal | 535165 | Not documented | 3 |
| ALR/Alpha_dup679 | Multi | No | — | — | Other | Other | chrY: 10093391-10104539 | − | 75 | 11149 | FAM197Y1 | Distal | 708699 | Not documented | 4 |
| | | | | | | Repeats | | | | | | | | | |
| CER_dup28 | Multi | No | — | — | Other | Other | chrY: 13337493-13341870 | + | 47 | 4378 | SLC9B1P1 | Distal | 154372 | Not documented | 5 |
| | | | | | | Repeats | | | | | | | | | |
| AluSx1_dup59209 | Multi/ | No | — | — | SINE | Alu | chrY: 21153222-21153521 | + | 47 | 300 | HSFY2 | Distal | 162675 | Not documented | 6 |
| | Uniquely | | | | | | | | | | | | | | |
| AluSx_dup75761 | Multi | No | — | — | SINE | Alu | chrY: 2355410-2355670 | − | 49 | 261 | SRY | Distal | 299227 | Not documented | 7 |
| AluSq2_dup30175 | Multi | No | — | — | SINE | Alu | chrY: 2356227-2356515 | − | 45 | 289 | SRY | Distal | 298382 | Not documented | 8 |
| MER4B_dup1052 | Multi | No | — | — | LTR | ERV1 | chrY: 23784589-23785241 | − | 77 | 653 | RBMY1A1 | Distal | 73378 | Not documented | 9 |
| LTR5_Hs_dup369 | Multi | No | — | — | LTR | ERVK | chrY: 26672935-26673902 | − | 3.6 | 968 | BPY2B | Distal | 79806 | Not documented | 5010 |
| LTR5_Hs_dup370 | Multi | No | — | — | LTR | ERVK | chrY: 27288495-27289462 | + | 4.1 | 968 | BPY2C | Distal | 79801 | Not documented | 1 |
| LTR4_dup47 | Multi | No | — | — | LTR | ERV1 | chrY: 3903820-3904377 | − | 76 | 558 | TGIF2LY | Distal | 455739 | Not documented | 2 |
| L3_dup27994 | Multi | No | — | — | LINE | CR1 | chrY: 5075960-5076044 | − | 130 | 85 | PCDH11Y | Proximal | 0 | Not documented | 3 |
| LTR5_Hs_dup358 | Multi | No | — | — | LTR | ERVK | chrY: 6616930-6617896 | + | 13 | 967 | AMELY | Distal | 116064 | Not documented | 4 |
| HUERS-P3-int_{—} | Multi | No | — | — | LTR | ERV1 | chrY: 8979290-8979514 | − | 120 | 225 | TSPY4 | Distal | 195560 | Not documented | 5 |
| dup366 | | | | | | | | | | | | | | | |
| MER4B_dup1013 | Multi | No | — | — | LTR | ERV1 | chrY: 9083418-9084070 | + | 77 | 653 | TSPY4 | Distal | 91004 | Not documented | 6 |
| L1HS_dup1060 | Multi | No | — | — | LINE | L1PA\|B\|x | chrY: 9428650-9429493 | + | 4.1 | 844 | FAM197Y1 | Distal | 43958 | Not documented | 7 |
| MER31-int_dup1419 | Multi | No | — | — | LTR | ERV1 | chrY: 9806195-9806504 | − | 150 | 310 | FAM197Y1 | Distal | 421503 | Not documented | 8 |
| HERVK22-int_{—} | Multi | No | — | — | LTR | ERVK | chrY: 9811447-9818183 | + | 71 | 6737 | FAM197Y1 | Distal | 426755 | EVE annotated | 9 |
| dup234 | | | | | | | | | | | | | | | |
| HERVH-int_dup3973 | Multi | No | — | — | LTR | ERV1 | chrY: 9885695-9887758 | − | 75 | 2064 | FAM197Y1 | Distal | 501003 | Not documented | 5020 |

Table 3 refers to the detailed identification of the peptides derived from neoplastic-TE signature by immunopeptidomics, corresponding to the neoantigenic peptides of SEQ ID NO: 1 to 370. The peptides are identified by their SEQ ID NO:; for example PEP:0001 corresponds to the peptide of SEQ ID NO: 1 in the attached sequence listing.

The column numbers refer to the following:
Column n° 1: Peptide_ID
Column n° 2: Analysis
Column n° 3: TE_Class
Column n° 4: TE_Category
Column n° 5: Median age_million
Column n° 6: Specificity
Column n° 7: n_Genomic_TEs_coding_peptides
Column n° 8: Peptide_sequence
Column n° 9: Group
Column n° 10: Immunogenicity_TD

TABLE 3

| 1 | 2 | 3 | 4 | 5 | 6 | 7 | 8 | 9 | 10 |
|---|---|---|---|---|---|---|---|---|---|
| PEP: 0001 | Multi | LINE | Other L1 | 124 | Non redundant | 1 | ADILTLRL | Group2 | — |
| PEP: 0002 | Uniquely | LINE | L1PA\|B\|x | 42.3 | Non redundant | 1 | AEAVPTSPPF | Group2 | — |
| PEP: 0003 | Multi/Uniquely | LINE | L1PA\|B\|x | 34.1 | Redundant | 11 | AGHAGLLR | Group1 | — |
| PEP: 0004 | Multi | Other | SVA | 19.5 | Redundant | 217 | AHYNLHLPA | Group1 | — |
| PEP: 0005 | Multi | Other | SVA | 19.5 | Redundant | 189 | AHYNLHLPAA | Group1 | — |
| PEP: 0006 | Uniquely | SINE | Alu | 47.3 | Redundant | 367 | AILLHQPPK | Group1 | — |
| PEP: 0007 | Multi/Uniquely | LINE | L1PA\|B\|x | 34.1 | Redundant | 40 | AILPKVINR | Group1 | — |
| PEP: 0008 | Multi/Uniquely | SINE | Alu | 57.3 | Redundant | 2830 | AILVPQPPK | Group2 | — |
| PEP: 0009 | Multi/Uniquely | LINE | L2 | 154 | Non redundant | 1 | ALPVYLLSK | Group2 | — |
| PEP: 0010 | Multi | LTR | ERV1 | 32.3 | Redundant | 101 | ALRAVTLTVK | Group1 | — |
| PEP: 0011 | Multi | DNA | DNA | 145 | Non redundant | 1 | ALSPTGNAK | Group2 | — |
| PEP: 0012 | Uniquely | Other | SVA | 20.9 | Redundant | 282 | ALWNRKAGKVGK | Group1 | — |
| PEP: 0013 | Uniquely | LINE | L1PA\|B\|x | 76.4 | Non redundant | 1 | ANLILSSY | Group1 | — |
| PEP: 0014 | Multi | Other | SVA | 11.4 | Redundant | 68 | APLLGRNPV | Group1 | — |
| PEP: 0015 | Multi/Uniquely | LINE | L1PA\|B\|x | 40.9 | Redundant | 54 | AQFELPGSF | Group1 | — |
| PEP: 0016 | Uniquely | Other | SVA | 20.7 | Redundant | 1060 | ARYNLHLPA | Group1 | — |
| PEP: 0017 | Uniquely | Other | SVA | 20.7 | Redundant | 984 | ARYNLHLPAA | Group1 | — |
| PEP: 0018 | Multi | LTR | ERVL-MaLR | 80 | Non redundant | 1 | ASASGDNLKK | Group2 | — |
| PEP: 0019 | Multi | LINE | L1PA\|B\|x | 38.6 | Redundant | 103 | ASEITSHIY | Group1 | — |
| PEP: 0020 | Multi | Other | Other Repeats | 58.6 | Redundant | 2 | ASITNKSLR | Group1 | — |
| PEP: 0021 | Uniquely | LTR | ERV1 | 46.4 | Redundant | 185 | ATAHSLGPR | Group1 | — |
| PEP: 0022 | Uniquely | LINE | L1PA\|B\|x | 42.7 | Non redundant | 1 | ATAIGQEKK | Group2 | — |
| PEP: 0023 | Multi | LTR | ERV1 | 66.8 | Non redundant | 1 | ATLQATTVYKK | Group2 | — |
| PEP: 0024 | Multi | LINE | L1PA\|B\|x | 2.27 | Redundant | 5462 | ATPRHIIVRF | Group1 | — |
| PEP: 0025 | Uniquely | LINE | L1PA\|B\|x | 37.3 | Redundant | 41 | ATPRHLIVRF | Group1 | — |
| PEP: 0026 | Multi | Other | SVA | 17.7 | Redundant | 304 | AVLSNRKGGNVGK | Group1 | — |
| PEP: 0027 | Multi | DNA | DNA | 130 | Non redundant | 1 | AYKDKDISFF | Group1 | — |
| PEP: 0028 | Uniquely | LINE | L1PA\|B\|x | 58.6 | Redundant | 10 | DAGLIEQV | Group1 | — |
| PEP: 0029 | Multi/Uniquely | LINE | L1PA\|B\|x | 1.82 | Redundant | 1950 | DQLEERVSA | Group1 | — |
| PEP: 0030 | Uniquely | LINE | L1PA\|B\|x | 79.5 | Non redundant | 1 | DQTVVGVWVY | Group2 | — |
| PEP: 0031 | Multi | Other | Other Repeats | 58.2 | Non redundant | 1 | EEFFKSAVSKQRFN | Group1 | — |
| PEP: 0032 | Uniquely | LINE | L1PA\|B\|x | 28.2 | Non redundant | 1 | EEKKREKNQIDA | Group1 | — |
| PEP: 0033 | Multi | LINE | L1PA\|B\|x | 38.6 | Redundant | 5 | EGAAVGAASTDL | Group1 | — |
| PEP: 0034 | Multi | LINE | L1PA\|B\|x | 29.1 | Redundant | 41 | EHLTHLHLR | Group1 | — |
| PEP: 0035 | Uniquely | LINE | L2 | 126 | Non redundant | 1 | EIIGGQSI | Group2 | — |
| PEP: 0036 | Uniquely | Other | Other Repeats | 142 | Non redundant | 1 | EIRDLQSHI | Group2 | — |
| PEP: 0037 | Uniquely | Other | Other Repeats | 61.8 | Redundant | 6 | EKELSSNR | Group1 | — |
| PEP: 0038 | Uniquely | LTR | ERV1 | 26.8 | Non redundant | 1 | EKKKDIVK | Group1 | — |
| PEP: 0039 | Uniquely | DNA | DNA | 116 | Non redundant | 1 | ELNHLHIFHI | Group1 | — |
| PEP: 0040 | Multi | LINE | Other L1 | 122 | Non redundant | 1 | ELVEQIENI | Group1 | — |
| PEP: 0041 | Multi | Other | Other Repeats | 84.5 | Redundant | 9 | ERMISLPILQ | Group1 | — |
| PEP: 0042 | Multi | DNA | DNA | 120 | Non redundant | 1 | ESFGQYWGVR | Group2 | — |
| PEP: 0043 | Multi | SINE | Alu | 55.5 | Redundant | 433 | ESHSVTQAGVR | Group1 | — |
| PEP: 0044 | Multi | SINE | Alu | 48.2 | Redundant | 45 | ESQHFGSPR | Group2 | — |
| PEP: 0045 | Multi | LTR | ERV1 | 62.7 | Non redundant | 1 | ESRQTGLSR | Group2 | — |
| PEP: 0046 | Multi/Uniquely | LTR | ERV1 | 67.3 | Redundant | 2 | ETLGKTLWR | Group1 | — |
| PEP: 0047 | Multi | SINE | Alu | 62.7 | Redundant | 217 | ETPSLKNK | Group2 | — |
| PEP: 0048 | Multi | LTR | ERV1 | 85.9 | Non redundant | 1 | ETTNPSIKK | Group2 | — |
| PEP: 0049 | Uniquely | SINE | MIR | 108 | Non redundant | 1 | EVASIKSKY | Group1 | — |
| PEP: 0050 | Uniquely | LINE | Other L1 | 104 | Non redundant | 1 | FFFFFWGCV | Group1 | — |
| PEP: 0051 | Multi/Uniquely | LINE | Other L1 | 113 | Redundant | 2 | FFLLPSTIS | Group2 | — |
| PEP: 0052 | Uniquely | LINE | L1PA\|B\|x | 44.5 | Redundant | 224 | FIDSMLFLS | Group1 | pMS16 |
| PEP: 0053 | Multi/Uniquely | LINE | L1PA\|B\|x | 10.9 | Redundant | 5048 | FIIFYCVYL | Group1 | pMS17 |
| PEP: 0054 | Uniquely | LINE | L1PA\|B\|x | 75.5 | Non redundant | 1 | FIIKGYQI | Group1 | — |
| PEP: 0055 | Uniquely | LINE | L1PA\|B\|x | 47.7 | Redundant | 45 | FIYLFKESA | Group1 | — |
| PEP: 0056 | Uniquely | LINE | L1PA\|B\|x | 2.27 | Redundant | 409 | FKYKWTKF | Group1 | — |
| PEP: 0057 | Multi/Uniquely | DNA | DNA | 97.3 | Non redundant | 1 | FLMQYHIFL | Group2 | pMS18 |
| PEP: 0058 | Multi | DNA | DNA | 130 | Non redundant | 1 | FLTEINSTV | Group1 | — |
| PEP: 0059 | Multi | LINE | L1PA\|B\|x | 30.5 | Redundant | 67 | FPAFLMIAI | Group1 | — |
| PEP: 0060 | Uniquely | DNA | DNA | 51.8 | Non redundant | 1 | FPLEKIVPR | Group2 | — |
| PEP: 0061 | Multi | LINE | L1PA\|B\|x | 50.5 | Non redundant | 1 | FPSFLPSPR | Group2 | — |
| PEP: 0062 | Multi/Uniquely | LINE | L1PA\|B\|x | 12.7 | Redundant | 291 | FSISSPAPV | Group1 | — |
| PEP: 0063 | Uniquely | LINE | L2 | 163 | Non redundant | 1 | FSTKIISVL | Group1 | — |
| PEP: 0064 | Uniquely | LINE | Other L1 | 74.5 | Non redundant | 1 | FTDSCKPTKY | Group1 | — |
| PEP: 0065 | Uniquely | LINE | L2 | 122 | Non redundant | 1 | FYDTVKVL | Group2 | — |
| PEP: 0066 | Uniquely | LINE | Other L1 | 71.8 | Non redundant | 1 | FYSDILTIFKL | Group1 | — |
| PEP: 0067 | Multi | LINE | L2 | 128 | Redundant | 2 | GEKWIWETF | Group2 | — |
| PEP: 0068 | Multi/Uniquely | LINE | Other L1 | 121 | Non redundant | 1 | GESQILQTR | Group1 | — |
| PEP: 0069 | Multi | Other | Other Repeats | 64.5 | Redundant | 2 | GFSMFVPHIGF | Group1 | — |
| PEP: 0070 | Uniquely | LINE | L2 | 126 | Non redundant | 1 | GGRHPIIKGL | Group1 | — |
| PEP: 0071 | Multi/Uniquely | LINE | Other L1 | 125 | Non redundant | 1 | GKIILSQEL | Group2 | — |
| PEP: 0072 | Uniquely | SINE | MIR | 117 | Non redundant | 1 | GLIISIFQMRK | Group2 | — |
| PEP: 0073 | Uniquely | LINE | L1PA\|B\|x | 99.1 | Non redundant | 1 | GNASWVDLGKIR | Group2 | — |
| PEP: 0074 | Multi/Uniquely | SINE | Alu | 52.3 | Redundant | 2533 | GQAGLKLLI | Group1 | — |
| PEP: 0075 | Multi/Uniquely | Other | SVA | 19.3 | Redundant | 2797 | GRLCPAAPTGK | Group1 | — |
| PEP: 0076 | Multi | LINE | Other L1 | 103 | Non redundant | 1 | GSIKRQGI | Group1 | — |
| PEP: 0077 | Multi/Uniquely | SINE | Alu | 49.8 | Redundant | 1350 | GSLQPLSPR | Group1 | — |
| PEP: 0078 | Uniquely | LINE | L2 | 151 | Non redundant | 1 | GTHLSPHYI | Group1 | — |
| PEP: 0079 | Multi | DNA | DNA | 54.5 | Non redundant | 1 | GVRELTSILK | Group1 | — |
| PEP: 0080 | Multi/Uniquely | LINE | L1PA\|B\|x | 2.95 | Redundant | 573 | HETISHTW | Group1 | — |
| PEP: 0081 | Uniquely | SINE | MIR | 135 | Redundant | 2 | HHSLSVNR | Group2 | — |
| PEP: 0082 | Multi | LTR | ERV1 | 66.8 | Non redundant | 1 | HILHALLNK | Group2 | — |
| PEP: 0083 | Uniquely | LINE | Other L1 | 74.5 | Non redundant | 1 | HIYEAGLTFK | Group1 | — |
| PEP: 0084 | Multi | LINE | Other L1 | 85.5 | Redundant | 6 | HLAEVVFAR | Group2 | — |
| PEP: 0085 | Multi | LINE | Other L1 | 85.5 | Redundant | 6 | HLAEVVFARF | Group2 | — |
| PEP: 0086 | Multi | LTR | ERV1 | 50.5 | Redundant | 2 | HLQEAYRIY | Group2 | — |
| PEP: 0087 | Multi | Other | Other Repeats | 70.9 | Redundant | 22 | HLTELNRS | Group2 | — |
| PEP: 0088 | Uniquely | LINE | L1PA\|B\|x | 49.1 | Non redundant | 1 | HPTESPKSI | Group2 | — |
| PEP: 0089 | Uniquely | SINE | Alu | 74.5 | Non redundant | 1 | HSLHAVATA | Group2 | — |
| PEP: 0090 | Multi | DNA | DNA | 43.6 | Redundant | 4 | HTVQWWLKK | Group2 | — |
| PEP: 0091 | Multi | LINE | Other L1 | 136 | Non redundant | 1 | HTYGFFTSK | Group1 | — |
| PEP: 0092 | Multi | SINE | Alu | 51.4 | Redundant | 2014 | HTYNPSTLGGR | Group1 | — |
| PEP: 0093 | Multi | Other | Other Repeats | 72.3 | Non redundant | 1 | HVHVHILQKE | Group1 | — |
| PEP: 0094 | Multi | LINE | Other L1 | 85.5 | Non redundant | 1 | HVISRAHTNK | Group2 | — |
| PEP: 0095 | Uniquely | LINE | Other L1 | 133 | Non redundant | 1 | IAAEITFTNS | Group1 | — |
| PEP: 0096 | Uniquely | LINE | Other L1 | 82.7 | Redundant | 2 | IDIDKIIQ | Group2 | — |
| PEP: 0097 | Multi | SINE | Alu | 60.5 | Redundant | 361 | ILAQCNLHL | Group1 | — |
| PEP: 0098 | Uniquely | LINE | Other L1 | 98.2 | Non redundant | 1 | ILDVLTPLSL | Group2 | — |
| PEP: 0099 | Multi/Uniquely | LINE | L1PA\|B\|x | 34.1 | Redundant | 49 | ILPKVINRF | Group1 | — |
| PEP: 0100 | Multi/Uniquely | LINE | L1PA\|B\|x | 2.27 | Redundant | 13172 | ILPKVIYRF | Group1 | — |
| PEP: 0101 | Multi | LINE | L1PA\|B\|x | 42.3 | Redundant | 56 | ILSSNLISL | Group1 | — |
| PEP: 0102 | Uniquely | Other | Other Repeats | 66.8 | Redundant | 4 | ILYEGQTLK | Group1 | — |
| PEP: 0103 | Uniquely | Other | Other Repeats | 160 | Redundant | 3 | INLKKKEL | Group2 | — |
| PEP: 0104 | Multi | Other | Other Repeats | 59.1 | Non redundant | 1 | IQICVLSTHI | Group1 | — |
| PEP: 0105 | Multi | LTR | ERV1 | 62.7 | Non redundant | 1 | IQNEIHAQR | Group2 | — |
| PEP: 0106 | Uniquely | LINE | Other L1 | 73.6 | Non redundant | 1 | IRQKPQLK | Group2 | — |
| PEP: 0107 | Uniquely | LTR | ERVK | 51.4 | Non redundant | 1 | IRQSSLLAK | Group2 | — |
| PEP: 0108 | Uniquely | LINE | Other L1 | 81.4 | Redundant | 72 | ISGLSTLFH | Group1 | — |
| PEP: 0109 | Uniquely | Other | Other Repeats | 49.1 | Redundant | 2 | ISKSTPNIY | Group1 | — |
| PEP: 0110 | Uniquely | LINE | L2 | 130 | Non redundant | 1 | ISLSRYFPISL | Group1 | — |
| PEP: 0111 | Uniquely | LINE | L1PA\|B\|x | 58.2 | Non redundant | 1 | ISPLSFLIIY | Group1 | — |
| PEP: 0112 | Multi/Uniquely | LINE | L1PA\|B\|x | 2.27 | Redundant | 37 | IVSAQNILK | Group1 | — |
| PEP: 0113 | Uniquely | LINE | L1PA\|B\|x | 24.1 | Redundant | 8057 | IVSAQNLLK | Group1 | — |
| PEP: 0114 | Multi/Uniquely | LINE | L2 | 122 | Non redundant | 1 | IYFSHLQYF | Group1 | — |
| PEP: 0115 | Multi | LINE | RTE | 168 | Non redundant | 1 | IYYPFINTK | Group2 | — |
| PEP: 0116 | Uniquely | LINE | L1PA\|B\|x | 30.5 | Redundant | 18 | KDLELEIR | Group1 | — |
| PEP: 0117 | Uniquely | LINE | L1PA\|B\|x | 42.3 | Non redundant | 1 | KDNDLHSIH | Group1 | — |
| PEP: 0118 | Uniquely | LINE | Other L1 | 86.8 | Non redundant | 1 | KDSFLYFLL | Group1 | — |
| PEP: 0119 | Uniquely | LINE | Other L1 | 61.4 | Non redundant | 1 | KDSIHHKL | Group2 | — |
| PEP: 0120 | Uniquely | LTR | ERVL-MaLR | 71.8 | Non redundant | 1 | KEEVIHALK | Group2 | — |
| PEP: 0121 | Uniquely | LINE | L2 | 130 | Non redundant | 1 | KGCPLKEK | Group1 | — |
| PEP: 0122 | Uniquely | LTR | ERV1 | 36.8 | Non redundant | 1 | KGLGPSPK | Group2 | — |
| PEP: 0123 | Uniquely | LTR | ERVL-MaLR | 111 | Non redundant | 1 | KGQWVTTI | Group1 | — |
| PEP: 0124 | Uniquely | LINE | Other L1 | 64.5 | Non redundant | 1 | KGSVAVNK | Group2 | — |
| PEP: 0125 | Multi | LINE | Other L1 | 141 | Redundant | 4 | KIFHFIQVK | Group2 | — |
| PEP: 0126 | Uniquely | LINE | L2 | 160 | Non redundant | 1 | KIFHNFQLP | Group2 | — |
| PEP: 0127 | Multi | LTR | ERV1 | 17.3 | Non redundant | 1 | KIHLHLAP | Group1 | — |
| PEP: 0128 | Uniquely | LINE | L1PA\|B\|x | 40 | Redundant | 9 | KIIKMAILPK | Group1 | — |
| PEP: 0129 | Multi/Uniquely | LINE | L1PA\|B\|x | 44.1 | Redundant | 96 | KIILNGQK | Group1 | — |
| PEP: 0130 | Uniquely | LINE | Other L1 | 69.1 | Redundant | 13 | KIINKILAK | Group1 | — |
| PEP: 0131 | Multi | LINE | Other L1 | 107 | Non redundant | 1 | KIKSFEKSNSWSWK | Group1 | — |
| PEP: 0132 | Multi | SINE | Alu | 51.8 | Redundant | 2282 | KIQKLARY | Group1 | — |
| PEP: 0133 | Multi/Uniquely | SINE | MIR | 158 | Non redundant | 1 | KITELGRI | Group2 | — |
| PEP: 0134 | Uniquely | LINE | Other L1 | 117 | Redundant | 25 | KLEKQEQIK | Group1 | — |
| PEP: 0135 | Multi/Uniquely | LTR | ERV1 | 42.3 | Redundant | 49 | KLIAGLIFLK | Group1 | — |
| PEP: 0136 | Multi | LINE | L1PA\|B\|x | 12.7 | Redundant | 9 | KLKTLQKI | Group1 | — |
| PEP: 0137 | Multi | Other | Other Repeats | 72.3 | Redundant | 3 | KLLCDDSI | Group1 | — |
| PEP: 0138 | Multi | LINE | Other L1 | 134 | Non redundant | 1 | KLNQKEYLQL | Group2 | — |
| PEP: 0139 | Uniquely | LINE | Other L1 | 79.1 | Non redundant | 1 | KMFLNICLMK | Group1 | — |
| PEP: 0140 | Uniquely | LINE | L1PA\|B\|x | 39.5 | Redundant | 35 | KMPKAVATK | Group2 | — |
| PEP: 0141 | Uniquely | LINE | Other L1 | 101 | Non redundant | 1 | KNKTLPAK | Group1 | — |
| PEP: 0142 | Uniquely | LINE | Other L1 | 109 | Redundant | 2 | KNVISLGK | Group1 | — |
| PEP: 0143 | Uniquely | LTR | ERVL | 76.8 | Non redundant | 1 | KPALLGFMK | Group1 | — |
| PEP: 0144 | Multi | LINE | Other L1 | 135 | Non redundant | 1 | KQFGEDRSLK | Group1 | — |
| PEP: 0145 | Multi/Uniquely | LINE | L1PA\|B\|x | 2.27 | Redundant | 9732 | KQWGKDSLFNK | Group1 | — |
| PEP: 0146 | Multi | LINE | L1PA\|B\|x | 62.3 | Redundant | 15 | KRLQELLIR | Group1 | — |
| PEP: 0147 | Multi | LTR | ERVK | 31.8 | Redundant | 9 | KRQGSLLGK | Group2 | — |
| PEP: 0148 | Multi | Other | Other Repeats | 72.3 | Redundant | 2 | KRVLPTAQSK | Group1 | — |
| PEP: 0149 | Uniquely | LINE | Other L1 | 122 | Redundant | 2 | KSAVISIT | Group2 | — |
| PEP: 0150 | Multi | LINE | Other L1 | 107 | Non redundant | 1 | KSFEKSNSW | Group1 | — |
| PEP: 0151 | Multi | LINE | Other L1 | 107 | Non redundant | 1 | KSFEKSNSWSWK | Group1 | — |
| PEP: 0152 | Uniquely | LINE | L1PA\|B\|x | 75.5 | Non redundant | 1 | KSRILVHPPP | Group1 | — |
| PEP: 0153 | Uniquely | LINE | L1PA\|B\|x | 47.7 | Non redundant | 1 | KSVQGFLK | Group1 | — |
| PEP: 0154 | Multi/Uniquely | DNA | DNA | 68.2 | Redundant | 330 | KSYYLRNTF | Group1 | — |
| PEP: 0155 | Uniquely | LINE | Other L1 | 80.9 | Redundant | 14 | KTAILNSLK | Group1 | — |
| PEP: 0156 | Multi | LINE | L1PA\|B\|x | 43.6 | Redundant | 11 | KTDRLLAKL | Group1 | — |
| PEP: 0157 | Multi/Uniquely | LTR | ERVL-MaLR | 89.1 | Non redundant | 1 | KTQEDGHLQAK | Group2 | — |
| PEP: 0158 | Multi | SINE | Alu | 78.6 | Redundant | 9 | KTSLDSIVK | Group2 | — |
| PEP: 0159 | Uniquely | LINE | L1PA\|B\|x | 88.6 | Non redundant | 1 | KVQLHCMFITALFSK | Group2 | — |
| PEP: 0160 | Multi | LINE | Other L1 | 97.7 | Redundant | 6 | LDSLILSHL | Group1 | — |
| PEP: 0161 | Uniquely | LINE | L2 | 126 | Non redundant | 1 | LEYLGPEL | Group1 | — |
| PEP: 0162 | Multi | LINE | L1PA\|B\|x | 50.5 | Non redundant | 1 | LFKLGGLCI | Group2 | — |
| PEP: 0163 | Multi/Uniquely | LINE | L1PA\|B\|x | 35.5 | Redundant | 107 | LFTLMMVSF | Group1 | — |
| PEP: 0164 | Multi/Uniquely | SINE | Alu | 45.5 | Non redundant | 1 | LGDRKTLISKK | Group2 | — |
| PEP: 0165 | Uniquely | LINE | L2 | 122 | Non redundant | 1 | LGVPCSQPGK | Group2 | — |
| PEP: 0166 | Multi/Uniquely | LINE | L1PA\|B\|x | 6.36 | Redundant | 14 | LHLIRKSI | Group1 | — |
| PEP: 0167 | Multi | DNA | DNA | 75.5 | Non redundant | 1 | LIAPHLQSK | Group2 | — |
| PEP: 0168 | Multi/Uniquely | LINE | L1PA\|B\|x | 36.4 | Redundant | 3884 | LIYTPTNSVK | Group1 | — |
| PEP: 0169 | Multi | LINE | L1PA\|B\|x | 8.18 | Redundant | 12253 | LLDIWVGSK | Group1 | — |
| PEP: 0170 | Uniquely | LINE | L2 | 132 | Redundant | 4 | LLFSFLFLI | Group1 | — |
| PEP: 0171 | Uniquely | LINE | Other L1 | 74.1 | Redundant | 21 | LLLSFNSSL | Group1 | — |
| PEP: 0172 | Uniquely | LINE | Other L1 | 104 | Non redundant | 1 | LLLVPICCWALS | Group1 | — |
| PEP: 0173 | Uniquely | LINE | L2 | 123 | Non redundant | 1 | LLQRTSGK | Group1 | — |
| PEP: 0174 | Multi | LINE | Other L1 | 113 | Redundant | 2 | LLSKFHLI | Group1 | — |
| PEP: 0175 | Multi | LTR | ERVK | 2.27 | Redundant | 7 | LMIGKRIL | Group1 | — |
| PEP: 0176 | Uniquely | LINE | Other L1 | 51.4 | Redundant | 99 | LMISDVEHLFM | Group1 | — |
| PEP: 0177 | Multi | LINE | Other L1 | 122 | Non redundant | 1 | LNKLKIHTF | Group1 | — |
| PEP: 0178 | Multi | LINE | L1PA\|B\|x | 26.4 | Redundant | 116 | LPDPLHFL | Group1 | — |
| PEP: 0179 | Uniquely | DNA | DNA | 48.2 | Non redundant | 1 | LPENLLQLL | Group1 | — |
| PEP: 0180 | Uniquely | LTR | ERV1 | 91.8 | Non redundant | 1 | LPHPIIEV | Group1 | — |
| PEP: 0181 | Uniquely | LTR | ERVL | 159 | Non redundant | 1 | LQNIMLIQY | Group2 | — |
| PEP: 0182 | Uniquely | DNA | DNA | 55.5 | Non redundant | 1 | LSDIMLLQ | Group2 | — |
| PEP: 0183 | Uniquely | LINE | L1PA\|B\|x | 43.2 | Redundant | 2 | LSEKELISL | Group1 | — |
| PEP: 0184 | Multi | LINE | L1PA\|B\|x | 50.5 | Redundant | 11 | LSFQSRCLL | Group1 | — |
| PEP: 0185 | Uniquely | LINE | L1PA\|B\|x | 40.9 | Redundant | 11 | LSLMDIGLVP | Group1 | — |
| PEP: 0186 | Uniquely | LINE | L1PA\|B\|x | 42.7 | Redundant | 50 | LSLPGYEIL | Group1 | — |
| PEP: 0187 | Uniquely | LINE | L1PA\|B\|x | 65 | Non redundant | 1 | LSSSPQCLLS | Group1 | — |
| PEP: 0188 | Multi/Uniquely | SINE | Alu | 78.2 | Non redundant | 1 | LSYFFLPK | Group2 | — |
| PEP: 0189 | Uniquely | LTR | ERV1 | 75 | Redundant | 8 | LTVSVQGIFK | Group1 | — |
| PEP: 0190 | Multi | LINE | Other L1 | 135 | Non redundant | 1 | LVYSKTVLK | Group2 | — |
| PEP: 0191 | Multi | Other | SVA | 15.5 | Redundant | 691 | LWNRKGGKVGK | Group1 | — |
| PEP: 0192 | Uniquely | LINE | Other L1 | 72.7 | Redundant | 3 | LYFPIYWHL | Group2 | — |
| PEP: 0193 | Multi | LTR | ERVL | 92.7 | Non redundant | 1 | MAYHGISQSPK | Group2 | — |
| PEP: 0194 | Multi | Other | SVA | 11.4 | Redundant | 76 | MESRSLSAQW | Group1 | — |
| PEP: 0195 | Uniquely | LTR | ERVK | 27.3 | Redundant | 4 | MIWLIVGAK | Group1 | — |
| PEP: 0196 | Multi | SINE | Alu | 32.3 | Redundant | 3 | MQFSCLSLPK | Group1 | — |
| PEP: 0197 | Multi | SINE | Alu | 40 | Redundant | 134 | MRFHHLGQA | Group1 | — |
| PEP: 0198 | Uniquely | LINE | Other L1 | 109 | Non redundant | 1 | MSQKGKLRI | Group1 | — |
| PEP: 0199 | Multi | LINE | L2 | 141 | Non redundant | 1 | MTFEPSFRR | Group2 | — |
| PEP: 0200 | Multi/Uniquely | Other | SVA | 30 | Redundant | 127 | MTMAVLSKR | Group1 | — |
| PEP: 0201 | Multi/Uniquely | SINE | Alu | 42.5 | Redundant | 2578 | MVKPPSLLK | Group1 | — |
| PEP: 0202 | Multi/Uniquely | LINE | L1PA\|B\|x | 28.4 | Redundant | 536 | NEIKEDTKKW | Group1 | — |
| PEP: 0203 | Multi/Uniquely | LINE | L1PA\|B\|x | 25.9 | Redundant | 5018 | NEIKEDTNKW | Group1 | — |
| PEP: 0204 | Uniquely | LINE | L1PA\|B\|x | 56.8 | Redundant | 75 | NLHSHQQRI | Group1 | — |
| PEP: 0205 | Uniquely | LINE | Other L1 | 69.1 | Redundant | 45 | NLISRVTTL | Group1 | — |
| PEP: 0206 | Uniquely | LINE | Other L1 | 104 | Non redundant | 1 | NQKIREIK | Group1 | — |
| PEP: 0207 | Multi | LINE | L1PA\|B\|x | 12.7 | Redundant | 261 | NQLEERVSA | Group1 | — |
| PEP: 0208 | Uniquely | LINE | L2 | 163 | Non redundant | 1 | NTVLNRLTF | Group1 | — |
| PEP: 0209 | Uniquely | LINE | L1PA\|B\|x | 44.1 | Redundant | 4 | PCIGLEHVSL | Group1 | — |
| PEP: 0210 | Multi/Uniquely | LINE | L1PA\|B\|x | 1.82 | Redundant | 2423 | PITGSEIVAI | Group1 | — |
| PEP: 0211 | Uniquely | LINE | Other L1 | 65.7 | Redundant | 108 | PLFPPLSK | Group1 | — |
| PEP: 0212 | Uniquely | LTR | ERV1 | 28.6 | Non redundant | 1 | PLRGVLQLLRQCVWS | Group1 | — |
| PEP: 0213 | Uniquely | Other | Other Repeats | 61.8 | Redundant | 4 | PMVEKEVSL | Group1 | — |
| PEP: 0214 | Multi | LINE | Other L1 | 133 | Non redundant | 1 | PNSTIILPI | Group2 | — |
| PEP: 0215 | Multi | LINE | L1PA\|B\|x | 21.8 | Non redundant | 1 | QDIISLTQL | Group1 | — |
| PEP: 0216 | Multi | Other | Other Repeats | 66.8 | Redundant | 2 | QIFSERFSL | Group1 | — |
| PEP: 0217 | Uniquely | LINE | Other L1 | 98.2 | Non redundant | 1 | QILSGISSY | Group1 | — |
| PEP: 0218 | Uniquely | LINE | L1PA\|B\|x | 50 | Non redundant | 1 | QIYRSMEQI | Group2 | — |
| PEP: 0219 | Multi/Uniquely | LINE | L1PA\|B\|x | 28.2 | Redundant | 2696 | QPLQKHAKL | Group1 | — |
| PEP: 0220 | Multi | LINE | L2 | 114 | Redundant | 3 | QPTFTEHLL | Group2 | — |
| PEP: 0221 | Uniquely | LTR | ERV1 | 23.2 | Redundant | 96 | QQIIVQTY | Group1 | — |
| PEP: 0222 | Multi | SINE | MIR | 167 | Non redundant | 1 | QRWELGLPHCTVS | Group2 | — |
| PEP: 0223 | Multi/Uniquely | LINE | L2 | 144 | Non redundant | 1 | QTATEPLVY | Group2 | — |
| PEP: 0224 | Multi | SINE | Alu | 78.6 | Redundant | 230 | QVIPLPWPPK | Group1 | — |
| PEP: 0225 | Multi/Uniquely | LTR | ERV1 | 127 | Redundant | 59 | QVLSPTSLK | Group1 | — |
| PEP: 0226 | Uniquely | Other | SVA | 28.2 | Redundant | 682 | RDQIVTVSV | Group1 | — |
| PEP: 0227 | Multi/Uniquely | Other | Other Repeats | 59.1 | Redundant | 3 | RFYNKSFSK | Group1 | — |
| PEP: 0228 | Uniquely | LINE | L1PA\|B\|x | 41.8 | Non redundant | 1 | RGRGTPPPPQP | Group2 | — |
| PEP: 0229 | Multi/Uniquely | LINE | L1PA\|B\|x | 1.82 | Redundant | 3903 | RIAKSILSQK | Group1 | — |
| PEP: 0230 | Multi/Uniquely | LINE | L1PA\|B\|x | 35 | Redundant | 231 | RIYNELKQISK | Group1 | — |
| PEP: 0231 | Multi/Uniquely | LINE | L1PA\|B\|x | 1.82 | Redundant | 8470 | RIYNELKQIYK | Group1 | — |
| PEP: 0232 | Multi | Other | SVA | 17.7 | Redundant | 39 | RLAAAPSGK | Group1 | — |
| PEP: 0233 | Multi/Uniquely | Other | SVA | 15.7 | Redundant | 1007 | RLCPAAPSEK | Group1 | — |
| PEP: 0234 | Multi/Uniquely | Other | SVA | 19.3 | Redundant | 2833 | RLCPAAPTGK | Group1 | — |
| PEP: 0235 | Multi | Other | SVA | 13.6 | Redundant | 84 | RLCPATPSEK | Group1 | — |
| PEP: 0236 | Multi/Uniquely | Other | SVA | 16.6 | Redundant | 506 | RLFPAAIPSRK | Group1 | — |
| PEP: 0237 | Multi | Other | SVA | 11.4 | Redundant | 287 | RLFPAAITSRK | Group1 | — |
| PEP: 0238 | Multi | DNA | DNA | 95 | Non redundant | 1 | RLIDMLTTK | Group2 | — |
| PEP: 0239 | Multi | LTR | ERV1 | 113 | Non redundant | 1 | RLPHYLLQK | Group2 | — |
| PEP: 0240 | Multi | LINE | L1PA\|B\|x | 5.45 | Redundant | 38 | RLSPLSLTQK | Group1 | — |
| PEP: 0241 | Multi/Uniquely | SINE | Alu | 50 | Redundant | 252 | RLTATSASGFK | Group1 | — |
| PEP: 0242 | Multi | LINE | L1PA\|B\|x | 21.8 | Redundant | 52 | RLWYQDDAGLTK | Group1 | — |
| PEP: 0243 | Multi | DNA | DNA | 130 | Non redundant | 1 | RMFDEQYSK | Group1 | — |
| PEP: 0244 | Uniquely | LINE | L1PA\|B\|x | 35.9 | Non redundant | 1 | RNTHAMVF | Group1 | — |
| PEP: 0245 | Multi | LTR | ERVL | 124 | Non redundant | 1 | RPGTTVGLRP | Group1 | — |
| PEP: 0246 | Uniquely | LINE | L2 | 140 | Non redundant | 1 | RQESRAGLRI | Group2 | — |
| PEP: 0247 | Multi/Uniquely | LINE | L1PA\|B\|x | 12.3 | Redundant | 5673 | RQPTKWEKI | Group1 | — |
| PEP: 0248 | Uniquely | LINE | L2 | 128 | Non redundant | 1 | RQQSVQWMW | Group2 | — |
| PEP: 0249 | Uniquely | LTR | ERVL | 49.1 | Redundant | 13 | RSIYLLHR | Group1 | — |
| PEP: 0250 | Multi/Uniquely | LTR | ERV1 | 28.6 | Redundant | 307 | RTLAVSVTALK | Group1 | — |
| PEP: 0251 | Multi | LTR | ERVL-MaLR | 50.9 | Non redundant | 1 | RTVSPINLFCK | Group1 | — |
| PEP: 0252 | Uniquely | LINE | L1PA\|B\|x | 31.8 | Redundant | 34 | RVFSNLVPFSR | Group1 | — |
| PEP: 0253 | Uniquely | LINE | L1PA\|B\|x | 45 | Non redundant | 1 | RWQGSHSVR | Group1 | — |
| PEP: 0254 | Uniquely | LINE | L2 | 138 | Redundant | 2 | SALLHHSL | Group2 | — |
| PEP: 0255 | Multi | Other | SVA | 14.1 | Redundant | 3 | SASARPRPSL | Group1 | — |
| PEP: 0256 | Multi/Uniquely | DNA | DNA | 130 | Non redundant | 1 | SASLHLLHK | Group1 | — |
| PEP: 0257 | Uniquely | LINE | L1PA\|B\|x | 88.2 | Non redundant | 1 | SFLSKAEMI | Group2 | — |
| PEP: 0258 | Uniquely | LINE | Other L1 | 114 | Redundant | 3 | SFVYLLEIF | Group2 | — |
| PEP: 0259 | Multi | DNA | DNA | 126 | Non redundant | 1 | SGAAVELVKE | Group2 | — |
| PEP: 0260 | Uniquely | LTR | ERVL | 111 | Non redundant | 1 | SGASTPMKL | Group2 | — |
| PEP: 0261 | Multi | LTR | ERVK | 4.55 | Redundant | 263 | SGFIPRHSI | Group1 | — |
| PEP: 0262 | Uniquely | LINE | L2 | 160 | Non redundant | 1 | SHQHLLIAR | Group2 | — |
| PEP: 0263 | Multi/Uniquely | Other | Other Repeats | 66.8 | Redundant | 2 | SHRIPEHSVW | Group1 | — |
| PEP: 0264 | Uniquely | LINE | L1PA\|B\|x | 43.2 | Redundant | 2 | SINITKMAI | Group2 | — |
| PEP: 0265 | Multi | DNA | DNA | 80 | Non redundant | 1 | SIYALLHQI | Group2 | — |
| PEP: 0266 | Multi | Other | Other Repeats | 66.8 | Redundant | 2 | SKSLSITK | Group1 | — |
| PEP: 0267 | Multi | LINE | L2 | 165 | Non redundant | 1 | SLDLEKQMSL | Group2 | — |
| PEP: 0268 | Multi | LTR | ERV1 | 22.3 | Redundant | 10 | SLFGGFFTR | Group1 | — |
| PEP: 0269 | Multi | Other | Other Repeats | 76.8 | Redundant | 9 | SLFHTEPF | Group2 | — |
| PEP: 0270 | Multi/Uniquely | SINE | Alu | 77.3 | Redundant | 67 | SLGNIERPY | Group2 | — |
| PEP: 0271 | Uniquely | LINE | L1PA\|B\|x | 35.5 | Redundant | 2 | SLHFIIYLV | Group1 | PMS22 |
| PEP: 0272 | Multi | SINE | Alu | 77.7 | Redundant | 8 | SLLQPETPGLK | Group2 | — |
| PEP: 0273 | Multi | SINE | Alu | 40.5 | Redundant | 368 | SLQPLPPMFK | Group1 | — |
| PEP: 0274 | Multi/Uniquely | Other | SVA | 13.2 | Redundant | 74 | SLSAWLPSLESEER | Group1 | — |
| PEP: 0275 | Uniquely | LINE | L1PA\|B\|x | 53.2 | Non redundant | 1 | SLSFFHWMSRS | Group2 | — |
| PEP: 0276 | Uniquely | LINE | L1PA\|B\|x | 41.4 | Redundant | 7 | SLSSKAPSAD | Group1 | — |
| PEP: 0277 | Multi | LTR | ERV1 | 120 | Non redundant | 1 | SLSSLWPVK | Group2 | — |
| PEP: 0278 | Uniquely | LINE | L1PA\|B\|x | 40 | Redundant | 2 | SLVNLNSK | Group1 | — |
| PEP: 0279 | Uniquely | LINE | L1PA\|B\|x | 79.5 | Non redundant | 1 | SLVQAWEL | Group1 | — |
| PEP: 0280 | Multi | Other | SVA | 16.4 | Redundant | 5 | SLYPAATPSGK | Group1 | — |
| PEP: 0281 | Multi | LTR | ERVL-MaLR | 80 | Non redundant | 1 | SMMPASASGDNLK | Group2 | — |
| PEP: 0282 | Multi | LTR | ERVL-MaLR | 80 | Non redundant | 1 | SMMPASASGDNLKK | Group2 | — |
| PEP: 0283 | Uniquely | LINE | Other L1 | 121 | Non redundant | 1 | SPLQKVNV | Group1 | — |
| PEP: 0284 | Multi | Other | SVA | 16.1 | Redundant | 456 | SPRSPSPSL | Group1 | — |
| PEP: 0285 | Multi | LINE | L2 | 167 | Non redundant | 1 | SQSLSLYLY | Group2 | — |
| PEP: 0286 | Multi | LINE | Other L1 | 153 | Non redundant | 1 | SRHIYVLIY | Group2 | — |
| PEP: 0287 | Uniquely | LTR | ERV1 | 46.4 | Non redundant | 1 | SSERAAHIK | Group2 | — |
| PEP: 0288 | Multi/Uniquely | Other | Other Repeats | 66.8 | Non redundant | 1 | SSFLESHVR | Group1 | — |
| PEP: 0289 | Multi | LINE | L2 | 140 | Redundant | 2 | SSLLSTQR | Group2 | — |
| PEP: 0290 | Uniquely | LINE | L1PA\|B\|x | 39.1 | Non redundant | 1 | SSLSSATLK | Group1 | — |
| PEP: 0291 | Multi | LTR | ERV1 | 120 | Non redundant | 1 | SSLSSLWPVK | Group2 | — |
| PEP: 0292 | Uniquely | SINE | Alu | 71.8 | Non redundant | 1 | STLGGKGART | Group1 | — |
| PEP: 0293 | Uniquely | DNA | DNA | 50 | Redundant | 266 | STLTGVWKK | Group1 | — |
| PEP: 0294 | Uniquely | DNA | DNA | 48.2 | Non redundant | 1 | SVKSTVITK | Group1 | — |
| PEP: 0295 | Uniquely | DNA | DNA | 48.2 | Non redundant | 1 | SVKSTVITKK | Group1 | — |
| PEP: 0296 | Uniquely | LINE | Other L1 | 80.5 | Non redundant | 1 | SVNIQWNI | Group2 | — |
| PEP: 0297 | Multi | LTR | ERVL | 102 | Non redundant | 1 | SVQDLLLGH | Group2 | — |
| PEP: 0298 | Uniquely | DNA | DNA | 48.2 | Non redundant | 1 | SVSPVSGEMKK | Group1 | — |
| PEP: 0299 | Uniquely | LINE | L1PA\|B\|x | 70 | Non redundant | 1 | SVWFFHISISL | Group2 | — |
| PEP: 0300 | Uniquely | Other | Other Repeats | 98.6 | Non redundant | 1 | SYLLKCFRI | Group2 | — |
| PEP: 0301 | Multi | SINE | MIR | 115 | Non redundant | 1 | SYLPSWTKL | Group2 | — |
| PEP: 0302 | Multi | SINE | MIR | 116 | Non redundant | 1 | SYLSGSQLSHL | Group1 | — |
| PEP: 0303 | Uniquely | LINE | L1PA\|B\|x | 31.8 | Redundant | 7319 | SYPAKLSFI | Group1 | — |
| PEP: 0304 | Uniquely | LINE | L1PA\|B\|x | 42.3 | Non redundant | 1 | TAKETIELTGN | Group1 | — |
| PEP: 0305 | Multi | LTR | ERV1 | 127 | Non redundant | 1 | TAKPPDLSR | Group1 | — |
| PEP: 0306 | Uniquely | LTR | ERV1 | 67.3 | Redundant | 4 | TALLWSLRK | Group1 | — |
| PEP: 0307 | Uniquely | LINE | Other L1 | 71.8 | Non redundant | 1 | TALTTILLSASM | Group1 | — |
| PEP: 0308 | Uniquely | LINE | L1PA\|B\|x | 30 | Redundant | 5 | TCKIVKTID | Group1 | — |
| PEP: 0309 | Uniquely | LINE | Other L1 | 101 | Redundant | 2 | TDLLLSLE | Group1 | — |
| PEP: 0310 | Uniquely | LTR | ERVL | 30.5 | Redundant | 169 | TEEEKTRAW | Group1 | — |
| PEP: 0311 | Multi | LTR | ERVK | 83.2 | Non redundant | 1 | TEQGPTGVTM | Group2 | — |
| PEP: 0312 | Multi | LINE | L2 | 173 | Non redundant | 1 | TFGNTELKI | Group2 | — |
| PEP: 0313 | Multi | LINE | L1PA\|B\|x | 107 | Non redundant | 1 | TGIPDREEEKI | Group2 | — |
| PEP: 0314 | Multi | SINE | Alu | 77.7 | Non redundant | 1 | TGPYPVAQAGLK | Group2 | — |
| PEP: 0315 | Multi | DNA | DNA | 88.6 | Non redundant | 1 | TGSEILIKH | Group2 | — |
| PEP: 0316 | Multi | SINE | Alu | 60 | Redundant | 20 | TGTTGARYH | Group1 | — |
| PEP: 0317 | Uniquely | LINE | L1PA\|B\|x | 90.9 | Non redundant | 1 | TIFGPSLAL | Group1 | — |
| PEP: 0318 | Multi | LTR | ERVL-MaLR | 129 | Non redundant | 1 | TILFEVCLA | Group2 | — |
| PEP: 0319 | Multi/Uniquely | SINE | Alu | 56.6 | Redundant | 558 | TLHSSLGNK | Group1 | — |
| PEP: 0320 | Multi | DNA | DNA | 130 | Non redundant | 1 | TLSDELTEI | Group1 | — |
| PEP: 0321 | Uniquely | SINE | Alu | 28.2 | Redundant | 4 | TMLARLAGT | Group2 | — |
| PEP: 0322 | Multi | LINE | Other L1 | 133 | Redundant | 3 | TNVLLVNI | Group2 | — |
| PEP: 0323 | Uniquely | LTR | ERVL | 66.8 | Non redundant | 1 | TPITINECSF | Group2 | — |
| PEP: 0324 | Multi/Uniquely | LINE | L1PA\|B\|x | 2.27 | Redundant | 5850 | TPRHIIVRF | Group1 | pMS5 |
| PEP: 0325 | Multi | LTR | ERVL-MaLR | 99.1 | Non redundant | 1 | TSQVFLSH | Group2 | — |
| PEP: 0326 | Uniquely | LINE | L2 | 123 | Non redundant | 1 | TSYSTQVK | Group1 | — |
| PEP: 0327 | Uniquely | LTR | ERV1 | 67.3 | Redundant | 3 | TTALLWSLRK | Group1 | — |
| PEP: 0328 | Multi | LINE | Other L1 | 133 | Non redundant | 1 | TTFYILLIF | Group1 | — |
| PEP: 0329 | Multi | LTR | ERV1 | 85.9 | Non redundant | 1 | TTNPSIKKR | Group2 | — |
| PEP: 0330 | Multi | LTR | ERVK | 83.2 | Redundant | 2 | TTQEAEKLLER | Group2 | — |
| PEP: 0331 | Multi/Uniquely | LTR | ERV1 | 67.3 | Non redundant | 1 | TTWSGNIMTK | Group1 | — |
| PEP: 0332 | Uniquely | LINE | Other L1 | 53.2 | Redundant | 7 | TVEKTVSSI | Group1 | — |
| PEP: 0333 | Uniquely | Other | Other Repeats | 49.1 | Non redundant | 1 | TVHQLSKY | Group1 | — |
| PEP: 0334 | Uniquely | LINE | Other L1 | 98.6 | Non redundant | 1 | TVLGLNQV | Group2 | — |
| PEP: 0335 | Uniquely | LTR | ERV1 | 36.8 | Non redundant | 1 | TVLKLLGKY | Group2 | — |
| PEP: 0336 | Multi/Uniquely | Other | Other Repeats | 68.9 | Redundant | 17 | TYILIEQF | Group1 | — |
| PEP: 0337 | Uniquely | LINE | L2 | 151 | Non redundant | 1 | VHYLGFHDI | Group1 | — |
| PEP: 0338 | Multi/Uniquely | SINE | Alu | 48.6 | Redundant | 8984 | VICLPWPPK | Group1 | — |
| PEP: 0339 | Multi/Uniquely | SINE | Alu | 49.5 | Redundant | 930 | VICPLRPPK | Group1 | — |
| PEP: 0340 | Multi | SINE | Alu | 53.2 | Redundant | 713 | VICSPRPPK | Group1 | — |
| PEP: 0341 | Multi | SINE | Alu | 55.5 | Redundant | 1418 | VIFLPQPPK | Group1 | — |
| PEP: 0342 | Uniquely | LINE | Other L1 | 51.4 | Redundant | 3 | VILVSYNGFGSI | Group1 | — |
| PEP: 0343 | Multi | SINE | MIR | 138 | Non redundant | 1 | VIRGWDSPI | Group1 | — |
| PEP: 0344 | Uniquely | LINE | L1PA\|B\|x | 18.6 | Non redundant | 1 | VISEGSVLSI | Group1 | — |
| PEP: 0345 | Multi | SINE | Alu | 24.5 | Redundant | 752 | VISPPRPPK | Group1 | — |
| PEP: 0346 | Multi | LINE | Other L1 | 112 | Non redundant | 1 | VLSSQVKII | Group2 | — |
| PEP: 0347 | Multi | Other | Other Repeats | 110 | Redundant | 23 | VLVGVSLPK | Group2 | — |
| PEP: 0348 | Multi | Other | SVA | 11.4 | Redundant | 8 | VLWNRNGGKVGK | Group1 | — |
| PEP: 0349 | Uniquely | LINE | L1PA\|B\|x | 22.3 | Redundant | 30 | VSFPSNSQNP | Group1 | — |
| PEP: 0350 | Multi | LINE | Other L1 | 118 | Non redundant | 1 | VSLSVRTTL | Group2 | — |
| PEP: 0351 | Multi | DNA | DNA | 95 | Redundant | 2 | VSSSLLSTK | Group2 | — |
| PEP: 0352 | Uniquely | LINE | L1PA\|B\|x | 58.6 | Non redundant | 1 | VSTGKLKTR | Group2 | — |
| PEP: 0353 | Multi | LTR | ERVL-MaLR | 103 | Non redundant | 1 | VTADLSSMRDPR | Group2 | — |
| PEP: 0354 | Multi | LINE | L2 | 129 | Non redundant | 1 | VTHRDIYVL | Group1 | — |
| PEP: 0355 | Uniquely | SINE | MIR | 124 | Non redundant | 1 | VTSIHRLYL | Group2 | — |
| PEP: 0356 | Multi | Other | Other Repeats | 58.6 | Non redundant | 1 | VTVVFWNLI | Group1 | — |
| PEP: 0357 | Multi | LINE | L1PA\|B\|x | 2.73 | Redundant | 28 | VVPVVPFH | Group1 | — |
| PEP: 0358 | Uniquely | LINE | L1PA\|B\|x | 49.1 | Redundant | 7 | VYIYIYML | Group1 | — |
| PEP: 0359 | Uniquely | Other | Other Repeats | 49.1 | Non redundant | 1 | VYNMYLVNF | Group1 | — |
| PEP: 0360 | Multi | Other | Other Repeats | 63.2 | Non redundant | 1 | VYSAHRVEPF | Group1 | — |
| PEP: 0361 | Multi | LINE | Other L1 | 99.5 | Non redundant | 1 | WGRVADKRELK | Group2 | — |
| PEP: 0362 | Multi | LINE | Other L1 | 125 | Non redundant | 1 | WKFSRKVT | Group1 | — |
| PEP: 0363 | Multi | LINE | L1PA\|B\|x | 38.6 | Redundant | 3 | WLASFPGIIY | Group1 | — |
| PEP: 0364 | Uniquely | LINE | Other L1 | 82.3 | Redundant | 2 | YIHNTIYI | Group2 | — |
| PEP: 0365 | Multi/Uniquely | LINE | Other L1 | 88.6 | Non redundant | 1 | YISLIKLSF | Group1 | — |
| PEP: 0366 | Uniquely | LINE | L1PA\|B\|x | 43.6 | Redundant | 8 | YLFKEPAFGF | Group2 | — |
| PEP: 0367 | Uniquely | LINE | Other L1 | 53.2 | Non redundant | 1 | YLIELCLQ | Group2 | — |
| PEP: 0368 | Uniquely | LTR | ERVL-MaLR | 117 | Redundant | 2 | YLRSHIMSL | Group1 | — |
| PEP: 0369 | Multi/Uniquely | DNA | DNA | 89.1 | Non redundant | 1 | YLTQYVPNI | Group2 | — |
| PEP: 0370 | Uniquely | LINE | L1PA\|B\|x | 165 | Non redundant | 1 | YPFSTSVLK | Group1 | - |

Table 4 refers to the detailed identification of the immunogenic peptides derived from neoplastic-TE signature by HLA-I binding predictions, corresponding to the neoantigenic peptides of SEQ ID NO: 371 to 380. The peptides are identified by their SEQ ID NO:; for example PEP:0371 corresponds to the peptide of SEQ ID NO: 371 in the attached sequence listing.

TABLE 4

| | | | | | Peptide_ | Immuno- |
|---|---|---|---|---|---|---|
| Peptide_ID | Analysis | Class | TE_Group | Age_million | sequence | genicity_ID |
| PEP: 0371 | Multi/Uniquely | DNA | DNA | 118.64 | ALSFWATRV | P3 |
| PEP: 0372 | Multi/Uniquely | DNA | DNA | 140.45 | LVIATFIKV | P4 |
| PEP: 0373 | Multi/Uniquely | DNA | DNA | 88.18 | FVVFKAPEV | P6 |
| PEP: 0374 | Multi/Uniquely | SINE | Alu | 55.91 | RLSHCLAWV | P8 |
| PEP: 0375 | Multi/Uniquely | LINE | CR1 | 151.36 | FVMPFCPSL | P11 |
| PEP: 0376 | Uniquely | DNA | DNA | 133.64 | LLLPGTPAV | P12 |
| PEP: 0377 | Multi/Uniquely | LTR | ERV1 | 74.55 | ALDQNSPGV | P14 |
| PEP: 0378 | Multi/Uniquely | LTR | ERV1 | 74.55 | SLMAVSMTI | P15 |
| PEP: 0379 | Multi/Uniquely | LTR | ERV1 | 74.55 | LMAKVSMTI | P16 |
| PEP: 0380 | Multi/Uniquely | LINE | CR1 | 151.36 | FLSIPFFFV | P17 |

Description of the sequences:

| Corresponding table and origin of the sequences | SEQ ID NOs |
|---|---|
| Table 3: Peptides derived from neoplastic-TE | 1-370 |
| signature by immunopeptidomics | |
| Table 4: Immunogenic peptides derived from | 371-380 |
| neoplastic-TE signature by HLA-I binding | |
| predictions | |
| Table 2: TE transcripts from neoplastic signature | 381-5020 |

1. A method for identifying a tumor cell TE signature comprising the steps of:
i) obtaining the single cell transcriptomic TE pattern of at least one tumor cell and the single cell TE transcriptomic pattern of at least one normal cell, and
ii) performing differential expression analysis of the TE transcriptomic pattern from said at least one tumor cell with respect to said at least one normal cell, and
iii) selecting the TE transcript sequences which are differentially expressed in said at least one tumor cell as compared to said at least one normal cell thereby obtaining a tumor cell TE signature.

2. The method of claim 1, wherein at step i) the single cell transcriptomic TE pattern is obtained by mapping the single-cell transcriptome to individual genomic TE occurrence.

3. A method for identifying TE-derived tumor neoantigenic peptides, the method comprising the steps of:
a) obtaining a tumor cell TE signature according to claim 1, and
b) in silico translating the TE transcript sequences from the tumor cell TE signature obtained at step a) to obtain TE-derived tumor peptides.

4. The method of claim 3 further comprising a step c) of identifying the TE derived peptides that bind at least one MHC molecule;
optionally wherein a library comprising the TE-derived peptide sequences identified at step b) is searched in the MHC ligandome from tumor cells and wherein matched peptides from the said MHC ligandome are selected, thus identifying MHC bound TE-derived peptides;
optionally wherein the TE-derived MHC bound peptides are further filtered against canonical proteins; and/or
optionally wherein the TE-encoded peptides which binds at least one MHC class I or II molecule of a subject with a KD binding affinity of less than 10′5 M are selected.

5. The method of claim 4, further comprising a step d) of selecting non-redundant TE-derived peptides;
optionally wherein this step is achieved by mapping the TE-derived peptides of step c) to the individual TE genomic location and selecting uniquely mapped TE.

6. An isolated tumor neoantigenic peptide sequence having at least 8 amino acids, wherein said neoantigenic peptide comprises a TE encoded sequence and binds at least one MHC class I or II molecule of a subject with a KD binding affinity of less than 10′5 M wherein said neoantigenic peptide further has one or both of the following properties:
the TE expression of said peptide is derepressed in a tumor cell as compared to non-tumor cells;
the peptide is encoded by a TE transcript sequence or a fragment thereof obtained according to claim 1;
and/or
the peptide is encoded by a TE transcript or a fragment thereof of any one of SEQ ID NO:381 to 5020;
optionally wherein the peptide comprises at least 8 amino acids, in particular 8-15, notably 8-12 amino acids and binds at least one MHC class I molecule of a subject or comprises from 13 to 25 amino acids and binds at least one MHC class II of a subject.

7. The neoantigenic peptide according to claim 6, comprising or consisting of any one of SEQ ID NO: 1 to 26 and 28 to 380 or a fragment thereof, optionally wherein the peptide is encoded by a single genomic TE.

8. The method of claim 1 wherein the tumor is glioblastoma tumor.

9. The neoantigenic peptide according to claim 6, wherein the TE is characterized by one or more of the following properties:
the TE is selected from TE over 50.106 years; optionally wherein the TE is selected from the LINE-1, SVA and ERVK TE subfamilies; optionally wherein the TE is selected from L1PA/B/x TEs;
the TE is selected from TEs over 50.106 years;
the TE is selected from TEs bearing an intact or nearly intact ORF;
the TE is selected from intronic or intergenic TEs; and/or
the TE is encoded by chromosome 7.

10. A population of autologous dendritic cells or antigen presenting cells that have been pulsed with one or more of the peptides as defined in claim 6 or transfected with a polynucleotide encoding one or more of the peptides as defined in claim 6.

11. A vaccine or immunogenic composition capable of rising a specific T-cell response comprising:
one or more neoantigenic peptides as defined in claim 6;
one or more polynucleotides encoding a neoantigenic peptide as defined in claim 6, optionally linked to a heterologous regulatory control nucleotide sequence; and/or
a population of antigen presenting cells that have been pulsed with one or more of the peptides of claim 6 or transfected with a polynucleotide encoding one or more of the peptides of claim 6.

12. An antibody, or an antigen-binding fragment thereof, a T cell receptor (TCR), or a chimeric antigen receptor (CAR) that specifically binds a neoantigenic peptide as defined in claim 6, optionally in association with an MHC molecule, with a Kd affinity of about 10′6 M or less;
optionally wherein the antibody is a multispecific antibody that further targets at least an immune cell antigen;
optionally wherein the immune cell is a T cell, a NK cell or a dendritic cell;
optionally wherein the targeted antigen is CD3, CD 16, CD30 or a TCR;
optionally wherein the antibody is a multispecific antibody that further targets at least an immune cell antigen;
and/or
optionally wherein the T cell receptor is made soluble and fused to an antibody fragment directed to a T cell antigen, optionally wherein the targeted antigen is CD3 or CD 16.

13. A polynucleotide encoding the neoantigenic peptide of claim 6, or an antibody, a CAR or a TCR that specifically binds a neoantigenic peptide of claim 6 or a vector comprising the polynucleotide.

14. An immune cell that specifically binds to one or more neoantigenic peptides of claim 6;
optionally wherein the immune cell is an allogenic or autologous cell selected from T cell, NK cell, CD4+/CD8+, TILs/tumor derived CD8 T cells, central memory CD8+ T cells, Treg, MAIT, and Y8 T cell;
and/or
optionally wherein the T cell comprises a T cell receptor that specifically binds one or more neoantigenic peptides of claim 6, or a TCR or a CAR that specifically binds a neoantigenic peptide of claim 6.

15. A method of treating cancer or inhibiting cancer cell proliferation comprising administering to a subject in need thereof, a neoantigenic peptide of claim 6,
a population of dendritic cells that have been pulsed with one or more of the neoantigenic peptides of claim 6 or transfected with a polynucleotide encoding one or more of the peptides as defined in claim 6,
a vaccine or immunogenic composition comprising a neoantigenic peptide of claim 6, a polynucleotide encoding a neoantigenic peptide of claim 6 or a vector comprising said polynucleotide,
an antibody, an antigen-binding fragment thereof, a CAR or a TCR that specifically binds a neoantigenic peptide as defined in claim 6,
or
an immune cell that specifically binds to one or more neoantigenic peptides of claim 6 optionally wherein the cancer is glioblastoma.

16. The neoantigenic peptide of claim 6, wherein the tumor is glioblastoma tumor.
